(19) United States
(12) Patent Application Publication
(10) Pub. No.: US 2025/0051354 A1
(43) Pub. Date: Feb. 13, 2025

(54) NITROGEN-CONTAINING COMPOUND, ORGANIC ELECTROLUMINESCENT DEVICE, AND ELECTRONIC APPARATUS

(71) Applicant: SHAANXI LIGHTE OPTOELECTRONICS MATERIAL CO., LTD., Xi'an (CN)

(72) Inventors: Xianbin Xu, Xi'an (CN); Lei Yang, Xi'an (CN); Youngkook Kim, Xi'an (CN)

(21) Appl. No.: 18/551,863

(22) PCT Filed: Feb. 17, 2023
(86) PCT No.: PCT/CN2023/076795

§ 371 Date: Sep. 21, 2023

(30) Foreign Application Priority Data
Apr. 15, 2022 (CN) 202210394816.1
May 16, 2022 (CN) 202210527743.9

(51) Int. Cl. C07D 495/14; C07B 59/00; C07D 498/14; C07D 513/14; C07D 519/00; H10K 71/10; H10K 85/60

(52) U.S. Cl. CPC C07D 495/14; C07B 59/004; C07D 498/14; C07D 513/14; C07D 519/00; H10K 71/10; H10K 85/615; H10K 85/654; H10K 85/656; H10K 85/6572; H10K 85/6574; H10K 85/6576; C07B 2200/05

(57) ABSTRACT

The present application relates to the technical field of organic electroluminescent materials, and provides a nitrogen-containing compound, an organic electroluminescent device including the nitrogen-containing compound, and an electronic apparatus. The nitrogen-containing compound of the present application has a core structure that is indolyl-fused phenothiazine/phenoxazine. The nitrogen-containing compound, when used as a host material of the luminescent layer of an organic electroluminescent device, can significantly improve the luminescence efficiency and service life of the device.

### CROSS-REFERENCE TO RELATED APPLICATIONS

This application is the U.S. National Stage of International Application No. PCT/CN2023/076795, filed on Feb. 17, 2023, which claims the benefit of Chinese patent application No. CN202210394816.1 filed on Apr. 15, 2022 and Chinese patent application No. CN202210527743.9 filed on May 16, 2022, the contents of each of which are incorporated herein by reference in their entirety as a part of this application.

### FIELD OF THE INVENTION

The present disclosure relates to the technical field of organic electroluminescent materials, and in particular to a nitrogen-containing compound, an organic electroluminescent device comprising the nitrogen-containing compound, and an electronic apparatus.

### BACKGROUND OF THE INVENTION

With the development of electronic technology and the advancement of material science, electronic devices used to achieve electroluminescence or photoelectric conversion have found an increasingly wide range of applications. An organic electroluminescent device (OLED) typically comprises a cathode and an anode that are disposed opposite to each other, and a functional layer disposed between the cathode and the anode. The functional layer consists of a plurality of organic or inorganic film layers, and generally comprises an organic luminescent layer, a hole transport layer, an electron transport layer, etc. When a voltage is applied to the cathode and the anode, an electric field is formed between the two electrodes. Under the influence of the electric field, electrons on the cathode side migrate to the electroluminescent layer, and holes on the anode side also migrate to the electroluminescent layer. The electrons and the holes recombine in the electroluminescent layer, forming excitons. The excitons in excited states release energy, causing the electroluminescent layer to emit light to the outside.

Main problems with existing organic electroluminescent devices lie in their service life and efficiency. As display screens become larger and larger, driving voltages are increased accordingly, which necessitates improvement in luminescence efficiency and current efficiency. It is therefore necessary to continue to develop new materials to further improve the performance of organic electroluminescent devices.

### SUMMARY OF THE INVENTION

Directed against the above problems with the existing technology, the present disclosure aims at providing a nitrogen-containing compound, an electroluminescent device comprising the nitrogen containing compound, and an electronic apparatus. The nitrogen-containing compound, when used in an organic electroluminescent device, can improve the performance of the device.

According to a first aspect of the present disclosure, a nitrogen-containing compound is provided. The nitrogen-containing compound has a structure shown in Formula 1:

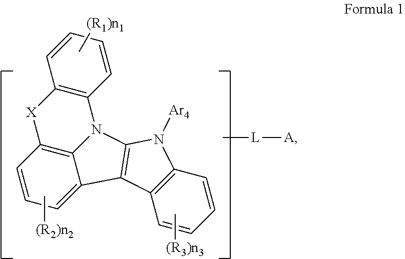

in which:
X is selected from S or O;
group A is selected from a structure shown in Formula a-1 or a structure shown in Formula a-2;

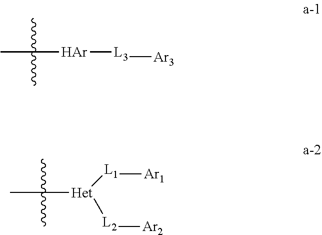

HAr is selected from substituted or unsubstituted arylene having 6 to 40 carbon atoms, and substituted or unsubstituted heteroarylene having 3 to 40 carbon atoms;
Het is a nitrogen-containing heteroarylene having 3 to 20 carbon atoms;
substituents in HAr are identical or different, and are each independently selected from deuterium, cyano, halogen, alkyl having 1 to 10 carbon atoms, deuterated alkyl having 1 to 10 carbon atoms, and aryl having 6 to 20 carbon atoms;
L, L₁, L₂, and L₃ are identical or different, and are each independently selected from single bond, substituted or unsubstituted arylene having 6 to 30 carbon atoms, and substituted or unsubstituted heteroarylene having 3 to 30 carbon atoms;
Ar₁ and Ar₂ are identical or different, and are each independently selected from substituted or unsubstituted aryl having 6 to 40 carbon atoms, and substituted or unsubstituted heteroaryl having 3 to 40 carbon atoms;
Ar₃ is selected from hydrogen, substituted or unsubstituted aryl having 6 to 40 carbon atoms, and substituted or unsubstituted heteroaryl having 3 to 40 carbon atoms;
Ar₄ is selected from substituted or unsubstituted aryl having 6 to 40 carbon atoms, and substituted or unsubstituted heteroaryl having 3 to 40 carbon atoms, or Ar₄ is single bond;
substituents in L, L₁, L₂, L₃, Ar₁, Ar₂, Ar₃, and Ar₄ are identical or different, and are each independently selected deuterium, cyano, halogen, alkyl having 1 to 10 carbon atoms, haloalkyl having 1 to 10 carbon atoms, deuterated alkyl having 1 to 10 carbon atoms, trialkylsilyl having 3 to 12 carbon atoms, triphenylsilyl, aryl having 6 to 20 carbon atoms, heteroaryl having 3 to 20 carbon atoms, phosphonyl having 6 to 20 carbon atoms, cycloalkyl having 3 to 10 carbon atoms, alkoxy having 1 to 10 carbon atoms, alkylthio having 1 to 10 carbon atoms, aryloxy having 6 to 20 carbon atoms, and arylthio having 6 to 20 carbon atoms; and optionally, in Ar₁, Ar₂, Ar₃, and Ar₄, any two adjacent substituents form a saturated or unsaturated 3 to 15-membered ring;
each R₁, each R₂, and each R₃ is identical or different, and is independently selected from deuterium, cyano, halogen, alkyl having 1 to 10 carbon atoms, haloalkyl having 1 to 10 carbon atoms, deuterated alkyl having 1 to 10 carbon atoms, trialkylsilyl having 3 to 12 carbon atoms, aryl having 6 to 20 carbon atoms, heteroaryl having 3 to 20 carbon atoms, and cycloalkyl having 3 to 10 carbon atoms; and optionally, any two adjacent groups form a benzene ring;
n₁ represents the number of R₁, and is selected from 0, 1, 2, 3, or 4; n₂ represents the number of R₂, and is selected from 0, 1, 2, or 3; and n₃ represents the number of R₃, and is selected from 0, 1, 2, 3, or 4.

According to a second aspect of the present disclosure, an organic electroluminescent device is provided. The organic electroluminescent device comprises an anode and a cathode that are disposed opposite to each other, and a functional layer disposed between the anode and the cathode. The functional layer comprises the nitrogen-containing compound described above.

According to a third aspect of the present disclosure, an electronic apparatus is provided. The electronic apparatus comprises the organic electroluminescent device described in the second aspect.

The structure of the nitrogen-containing compound of the present disclosure has a core structure that is indolyl-fused phenothiazine/phenoxazine. The sulfur or oxygen atom in indolophenothiazine/indolophenoxazine has two pairs of lone pair electrons, which can give the core structure an excellent hole transport ability. When the core structure is linked with an aryl group or an electron-rich heteroaryl group, the compound can have an enhanced hole transport ability; and this type of compound is suitable for use as a hole-transport type host material in a mixed-type host material. When the core structure is linked with a nitrogen-containing heteroarylene group having electron transport characteristics, the compound can have both excellent hole transport performance and excellent electron transport performance; and this type of compounds are suitable for use as a single-type host material. The compound of the present disclosure, whenever used as a hole transport-type host material in a mixed-type host material or as a single-type host material, can improve the balance of charge carriers in a luminescent layer, expand a region where the carriers recombine, improve the generation and utilization efficiency of excitons as well as the luminescence efficiency and the service life of an electroluminescent device.

### BRIEF DESCRIPTION OF THE DRAWINGS

The accompanying drawings are intended to provide a further understanding of the present disclosure and form a part of the specification. The accompanying drawings, together with the following specific embodiments, are used to illustrate the present disclosure, but do not constitute any limitation to the present disclosure.

FIG. 1 is a schematic structural diagram of an organic electroluminescent device according to an embodiment of the present disclosure.

FIG. 2 is a schematic structural diagram of an electronic apparatus according to an embodiment of the present disclosure.

List of Reference Signs

| | | | |
|---|---|---|---|
| 100: anode | 200: cathode | 300: functional layer | 310: hole injection layer |
| 321: first hole transport layer | 322: second hole transport layer | | |
| 330: organic luminescent layer | 340: electron transport layer | | |
| (light-emitting layer) | | | |
| 350: electron injection layer | 400: electronic apparatus | | |

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present disclosure more comprehensive and complete, and to communicate the concepts of these exemplary embodiments fully to those skilled in the art. Features, structures, or characteristics described can be combined in one or more embodiments in any suitable manner. In the following description, many specific details are provided to give a full understanding of the embodiments of the present disclosure.

The present disclosure, in a first aspect, provides a nitrogen-containing compound. The nitrogen-containing compound has a structure shown in Formula 1:

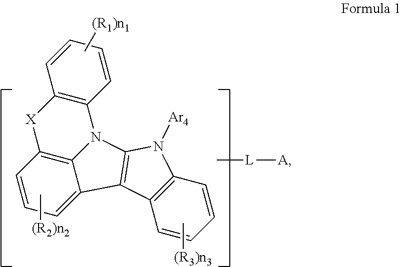

in which:

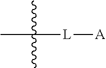

is linked to any carbon atom or any nitrogen atom in

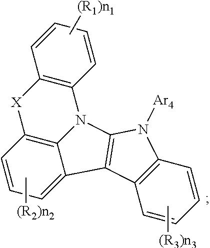

X is selected from S or O;
group A is selected from a structure shown in Formula a-1 or a structure shown in Formula a-2;

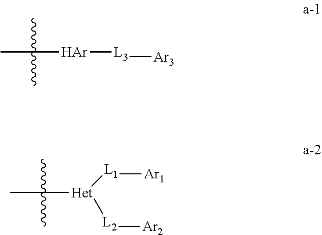

HAr is selected from substituted or unsubstituted arylene having 6 to 40 carbon atoms, and substituted or unsubstituted heteroarylene having 3 to 40 carbon atoms;
Het is a nitrogen-containing heteroarylene having 3 to 20 carbon atoms;
substituents in HAr are identical or different, and are each independently selected from deuterium, cyano, halogen, alkyl having 1 to 10 carbon atoms, deuterated alkyl having 1 to 10 carbon atoms, and aryl having 6 to 20 carbon atoms;
L, L₁, L₂, and L₃ are identical or different, and are each independently selected from single bond, substituted or unsubstituted arylene having 6 to 30 carbon atoms, and substituted or unsubstituted heteroarylene having 3 to 30 carbon atoms;
Ar₁ and Ar₂ are identical or different, and are each independently selected from substituted or unsubstituted aryl having 6 to 40 carbon atoms, and substituted or unsubstituted heteroaryl having 3 to 40 carbon atoms;
Ar₃ is selected from hydrogen, substituted or unsubstituted aryl having 6 to 40 carbon atoms, and substituted or unsubstituted heteroaryl having 3 to 40 carbon atoms;
Ar₄ is selected from substituted or unsubstituted aryl having 6 to 40 carbon atoms, and substituted or unsubstituted heteroaryl having 3 to 40 carbon atoms, or Ar₄ is single bond;
when

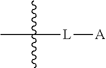

is linked to the site marked by [symbol] in

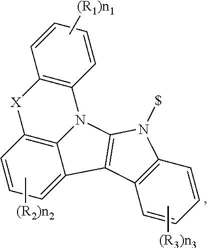

Ar₄ is single bond;
substituents in L, L₁, L₂, L₃, Ar₁, Ar₂, Ar₃, and Ar₄ are identical or different, and are each independently selected from the group consisting of deuterium, cyano, halogen, alkyl having 1 to 10 carbon atoms, haloalkyl having 1 to 10 carbon atoms, deuterated alkyl having 1 to 10 carbon atoms, trialkylsilyl having 3 to 12 carbon atoms, triphenylsilyl, aryl having 6 to 20 carbon atoms, heteroaryl having 3 to 20 carbon atoms, phosphonyl having 6 to 20 carbon atoms, cycloalkyl having 3 to 10 carbon atoms, alkoxy having 1 to 10 carbon atoms, alkylthio having 1 to 10 carbon atoms, aryloxy having 6 to 20 carbon atoms, and arylthio having 6 to 20 carbon atoms; and optionally, in Ar₁, Ar₂, Ar₃, and Ar₄, any two adjacent substituents form a saturated or unsaturated 3 to 15-membered ring;
each R₁, each R₂, and each R₃ is identical or different, and is independently selected from the group consisting of deuterium, cyano, halogen, alkyl having 1 to 10 carbon atoms, haloalkyl having 1 to 10 carbon atoms, deuterated alkyl having 1 to 10 carbon atoms, trialkylsilyl having 3 to 12 carbon atoms, aryl having 6 to 20 carbon atoms, heteroaryl having 3 to 20 carbon atoms, and cycloalkyl having 3 to 10 carbon atoms; and optionally, any two adjacent groups form a benzene ring, wherein said any two adjacent groups are any two adjacent R₁ and/or any two adjacent R₂ and/or any two adjacent R₃;
n₁ represents the number of R₁, and is selected from 0, 1, 2, 3, or 4; n₂ represents the number of R₂, and is selected from 0, 1, 2, or 3; n₃ represents the number of R₃, and is selected from 0, 1, 2, 3, or 4.

Optionally, any two adjacent R₁ form a benzene ring.

Optionally, any two adjacent R₂ form a benzene ring.

Optionally, any two adjacent R₃ form a benzene ring.

Optionally, in Formula 1,

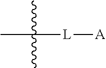

is linked to any linkable carbon atom or nitrogen atom in

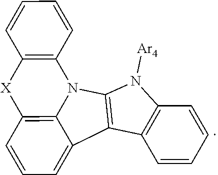

In Formula 1 above, when Ar₄ is single bond, the nitrogen atom

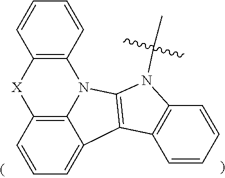

connected with Ar₄ links to

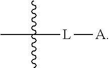

In the present disclosure, the term “saturated or unsaturated ring”, such as a saturated or unsaturated 3 to 15-membered ring, includes saturated carbon rings, saturated heterocyclic rings, partially unsaturated carbon rings, partially unsaturated heterocyclic rings, aromatic carbon rings, and aromatic heterocyclic rings. When a ring is prefixed with n-membered, n is an integer, indicating that the number of ring atoms of the ring is n. For example, a 3 to 15-membered ring represents a ring with 3-15 ring atoms, i.e., a ring with 3, 4, 5, 6, 7, 8, 9, 10, 11, 12, 13, 14, or 15 ring atoms.

In the present disclosure, the term “optional” or “optionally” means that the subsequently described event or circumstance may or may not occur. As an example, “optionally, any two adjacent substituents form a ring” means that the two adjacent substituents may or may not form a ring, i.e., including instances where the two adjacent substituents form a ring and instances where the two adjacent substituents do not form a ring. As another example, “optionally, in Ar₁, Ar₂, Ar₃, and Ar₄, any two adjacent substituents form a ring” means that any two adjacent substituents in Ar₁, Ar₂, Ar₃, and Ar₄ are linked to each other to form a ring, or any two adjacent substituents Ar₁, Ar₂, Ar₃, and Ar₄ may exist independently. The mentioning of “any two adjacent” may involve instances where there are two substituents on a same atom and also instances where there is one substituent on each of two adjacent atoms. When there are two substituents on a same atom, the two substituents, together with the atom to which they are attached, may form a saturated or unsaturated spiro ring; and when there is one substituent on each of two adjacent atoms, the two substituents may be fused into a ring.

In the present disclosure, the term “and/or” is used to connect two features, and it indicates that the two features both appear or either of the two features appears.

In the present disclosure, the expression “each . . . independently” may be used interchangeably with the expressions “ . . . independently”, and “ . . . each independently”, and all these expressions should be interpreted in a broad sense. They can not only mean that, for same symbols in a same group, the selection of a specific option for one of the symbols and the selection of a specific option for another one of the symbols do not affect each other, but also mean that for same symbols in different groups, the selection of a specific option for one of the symbols and the selection of a specific option for another one of the symbols do not affect each other. Taking

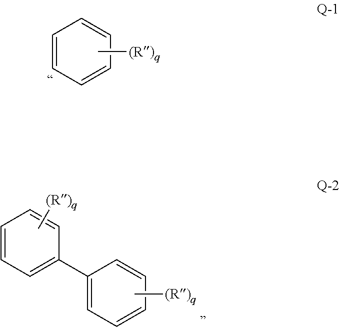

an example, each q is independently selected from 0, 1, 2, or 3, and each R″ is independently selected from hydrogen, deuterium, fluorine, or chlorine, which means: in Formula Q-1, there are q substituents R″ on the benzene ring, wherein each of the substituent R″ may be identical or different, with the selection of an option for one of the substituents R″ and the selection of an option for another one of the substituents R″ not affecting each other; and in Formula Q-2, there are q substituents R″ on each of the two benzene rings of biphenyl, wherein the number q of the substituent R″ on one benzene ring and the number q of the substituent R″ on the other benzene ring may be identical or different, and each substituent R″ may be identical or different, with the selection of an option for one of the substituents R″ and the selection of an option for another one of the substituents R″ not affecting each other.

In the present disclosure, the term “substituted or unsubstituted” means that the functional group defined by the term may or may not have a substituent (hereinafter referred to as Rc for ease of description). For example, “substituted or unsubstituted aryl” refers to an aryl group with a substituent Rc or an unsubstituent aryl group. The above-mentioned substituents, namely Rc, may be, for example, deuterium, halogen, cyano, heteroaryl, aryl, trialkylsilyl, alkyl, haloalkyl, cycloalkyl, etc. The number of the substitutes may be one or more.

In the present disclosure, the term “more” means two or more than two, for example, two, three, four, five, six, etc.

Hydrogen atoms in the structure of the compound of the present disclosure include various isotopic atoms of hydrogen element, such as hydrogen (H), deuterium (D), or tritium (T).

In the present disclosure, the number of carbon atoms of a substituted or unsubstituted functional group refers to the total number of carbon atoms. For example, if L₁ is a substituted arylene group having 12 carbon atoms, then the total number of carbon atoms of the arylene group and substituents thereon is 12.

In the present disclosure, “aryl” refers to any functional group or substituent group derived from an aromatic carbon ring. An aryl group may be a monocyclic aryl group (e.g., phenyl) or a polycyclic aryl group. In other words, an aryl group may be a monocyclic aryl group, a fused aryl group, two or more monocyclic aryl groups linked by a carbon-carbon bond, a monocyclic aryl group and a fused aryl group linked by a carbon-carbon bond, two or more fused aryl groups linked by a carbon-carbon bond, or a spirocyclic system containing a monocyclic aryl or a fused aryl group formed by sharing a carbon atom. That is, unless otherwise specified, two or more aromatic groups linked by a carbon-carbon bond may also be regarded as an aryl group in the present disclosure. Among them, fused aryl groups may include, for example, bicyclic fused aryl groups (e.g., naphthyl), tricyclic fused aryl groups (e.g., phenanthryl, fluorenyl, anthryl) and the like. Examples of aryl groups may include, but are not limited to, phenyl, naphthyl, fluorenyl, spirodifluorenyl, anthryl, phenanthryl, biphenyl, terphenyl, triphenylene

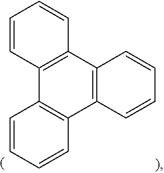 [SMILES: c1ccc2c(c1)c1ccccc1c1ccccc21]

perylenyl, 9,10-benzophenanthryl, pyrenyl, benzofluoranthryl, chrysenyl, etc.

In the present disclosure, “arylene” refers to a divalent or polyvalent group formed by further removing one or more hydrogen atoms from an aryl group.

In the present disclosure, terphenyl groups include

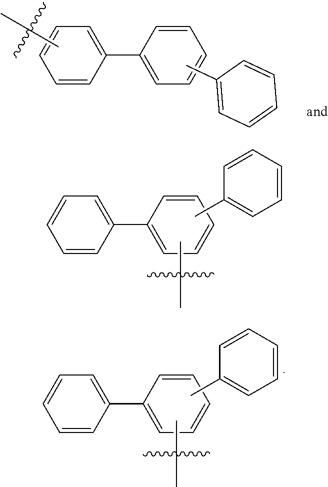

In the present disclosure, the number of carbon atoms of a substituted aryl group is the total number of carbon atoms of the aryl group and substituents on the aryl group. For example, a substituted aryl group having 18 carbon atoms means that the total number of carbon atoms of the aryl group and substituents thereon is 18.

In the present disclosure, the number of carbon atoms of a substituted or unsubstituted aryl group (arylene) may be 6, 8, 10, 12, 13, 14, 15, 16, 17, 18, 19, 20, 21, 22, 23, 24, 25, 26, 28, 30, 31, 33, 34, 35, 36, 38, or 40. In some embodiments, a substituted or unsubstituted aryl group is substituted or unsubstituted aryl having 6 to 40 carbon atoms. In other embodiments, a substituted or unsubstituted aryl group is substituted or unsubstituted aryl having 6 to 30 carbon atoms. In other embodiments, a substituted or unsubstituted aryl group is substituted or unsubstituted aryl having 6 to 25 carbon atoms. In other embodiments, a substituted or unsubstituted aryl group is substituted or unsubstituted aryl having 6 to 15 carbon atoms.

In the present disclosure, “fluorenyl” may be substituted by one or more substituents. In the case where the above fluorenyl is substituted, the substituted fluorenyl may be, but is not limited to,

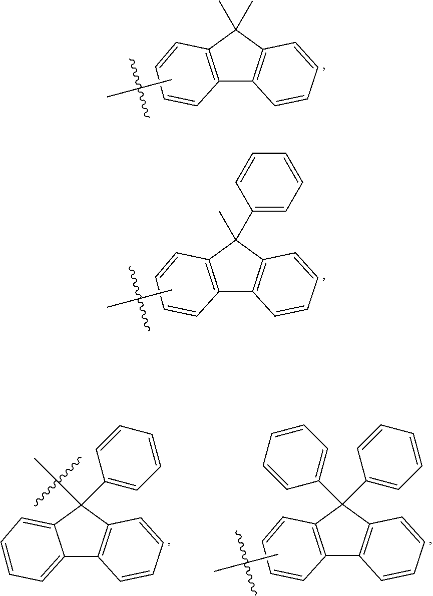

and the like.

In the present disclosure, aryl groups, as substituents of L, L₁, L₂, L₃, Ar₁, Ar₂, Ar₃, and Ar₄, may be, but are not limited to, phenyl, naphthyl, phenanthryl, biphenyl, fluorenyl, dimethylfluorenyl, etc.

In the present disclosure, “heteroaryl” refers to a monovalent aromatic ring containing 1, 2, 3, 4, 5, or 6 heteroatoms or a derivative thereof. The heteroatoms may be one or more selected from B, O, N, P, Si, Se, and S. A heteroaryl group may be a monocyclic heteroaryl group or polycyclic heteroaryl group. In other words, a heteroaryl group may be a single aromatic ring system, or a plurality of aromatic ring systems linked by carbon-carbon bonds, with any of the aromatic ring systems being an aromatic monocyclic ring or a fused aromatic ring, or may be a spirocyclic system containing a monocyclic aromatic ring or a fused aromatic ring formed by sharing a carbon atom. For example, heteroaryl groups may include, but are not limited to, thienyl, furyl, pyrryl, imidazolyl, thiazolyl, oxazolyl, oxadiazolyl, triazolyl, pyridinyl, bipyridinyl, pyrimidyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothiophenyl, benzofuranyl, phenanthrolinyl, isoxazolinyl, thiadiazolyl, phenothiazinyl, silafluorenyl, dibenzofuranyl, N-phenylcarbazolyl, N-pyridylcarbazolyl, N-methylcarbazolyl, etc.

In the present disclosure, a heteroarylene group is a divalent or polyvalent group formed by further removing one or more hydrogen atoms from a heteroaryl group.

In the present disclosure, the number of carbon atoms of a substituted or unsubstituted heteroaryl (heteroarylene) group may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments, a substituted or unsubstituted heteroaryl group is substituted or unsubstituted heteroaryl having 3 to 40 carbon atoms. In other embodiments, a substituted or unsubstituted heteroaryl group is substituted or unsubstituted heteroaryl having 3 to 30 carbon atoms. In other embodiments, a substituted or unsubstituted heteroaryl group is substituted or unsubstituted heteroaryl having 5 to 12 carbon atoms.

In the present disclosure, heteroaryl groups, as substituents of L, L₁, L₂, L₃, Ar₁, Ar₂, Ar₃, and Ar₄, may be, but are not limited to, for example, pyridyl, carbazolyl, quinolyl, isoquinolyl, phenanthrolinyl, benzoxazolyl, benzothiazolyl, benzimidazolyl, dibenzothienyl, and dibenzofuranyl.

In the present disclosure, a substituted heteroaryl group may mean that one or more hydrogen atoms in the heteroaryl group are substituted by a group such as a deuterium atom, a halogen, —CN (cyano), aryl, heteroaryl, trialkylsilyl, alkyl, cycloalkyl, haloalkyl, etc. It should be appreciated that the number of carbon atoms of a substituted heteroaryl group is the total number of carbon atoms of the heteroaryl group and substituents on the heteroaryl group.

In the present disclosure, alkyl groups having 1 to 10 carbon atoms may include straight-chain alkyl groups having 1 to 10 carbon atoms and branched-chain alkyl groups having 3 to 10 carbon atoms. For example, the number of carbon atoms of an alkyl group may be, for example, 1, 2, 3, 4, 5, 6, 7, 8, 9, or 10. Specific examples of alkyl groups include, but are not limited to, methyl, ethyl, n-propyl, isopropyl, n-butyl, isobutyl, tert-butyl, n-pentyl, isopentyl, neopentyl, n-hexyl, etc.

In the present disclosure, “halogen” may be, for example, fluorine, chlorine, bromine, or iodine.

In the present disclosure, specific examples of “trialkylsilyl” include, but are not limited to, trimethylsilyl, triethylsilyl, etc.

In the present disclosure, “haloalkyl” refers to an alkyl group with one or more halogen substituents. Examples of haloalkyl groups include, but are not limited to, trifluoromethyl.

In the present disclosure, the number of a cycloalkyl group having 3 to 10 carbon atoms may be, for example, 3, 4, 5, 6, 7, 8, or 10. Specific examples of cycloalkyl groups include, but are not limited to, cyclopentyl, cyclohexyl, adamantly, etc.

In the present disclosure, a nitrogen-containing heteroarylene group having 3 to 20 carbon atoms refers to a heteroarylene group having 3-20 carbon atoms and at least one nitrogen atom.

In the present disclosure, “ [symbol] ”, “ [symbol] ”, “ [symbol] ”, “ [symbol] ”, and “ [symbol] ” are chemical bonds linked with other groups, and marks on the bonds are only intended to distinguish the bonds from one another.

In the present disclosure, anon-orientating linkage bond is single bond “ [symbol] ” extending from a ring system, and it indicates that the linkage bond can be linked at one end thereof to any site in the ring system through which the bond passes, and linked at the other end thereof to the rest of the compound molecule. For example, as shown in Formula (f) below, the naphthalyl group represented by Formula (f) is linked with other sites of the molecule via two non-orientating linkage bonds passing through the two rings, which indicates any of possible linkages shown in Formulae (f-1) to (f-10):

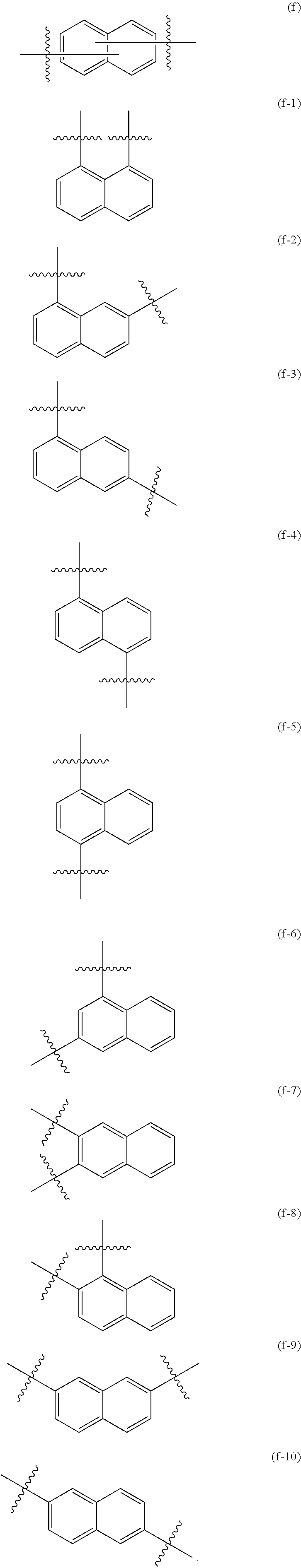

As another example, as shown in Formula (X′) below, the dibenzofuranyl group represented by Formula (X′) is linked with other sites of the molecule via a non-orientating linkage bond extending from the center of a side benzene ring, which indicates any of possible linkages shown in Formulae (X′-1) to (X′-4):

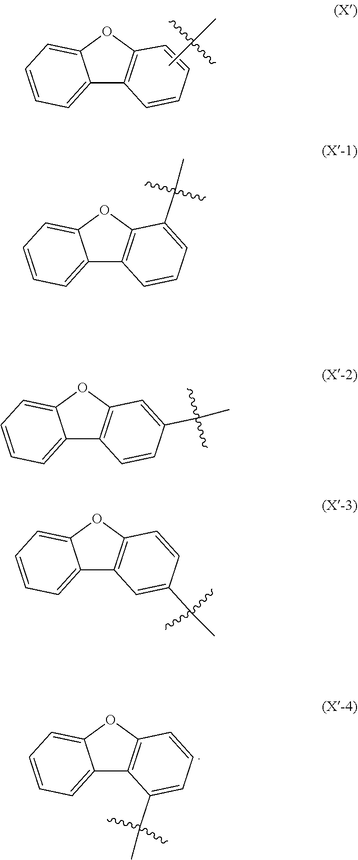

A non-orientating substituent in the present disclosure refers to a substituent linked via single bond extending from the center of a ring system, and it means that the substituent may be linked with any possible site in the ring system. For example, as shown in Formula (Y) below, the substituent R′ represented by Formula (Y) is linked with a quinoline ring via a non-orientating linkage bond, which indicates any of possible linkages shown in Formulae (Y-1) to (Y-7):

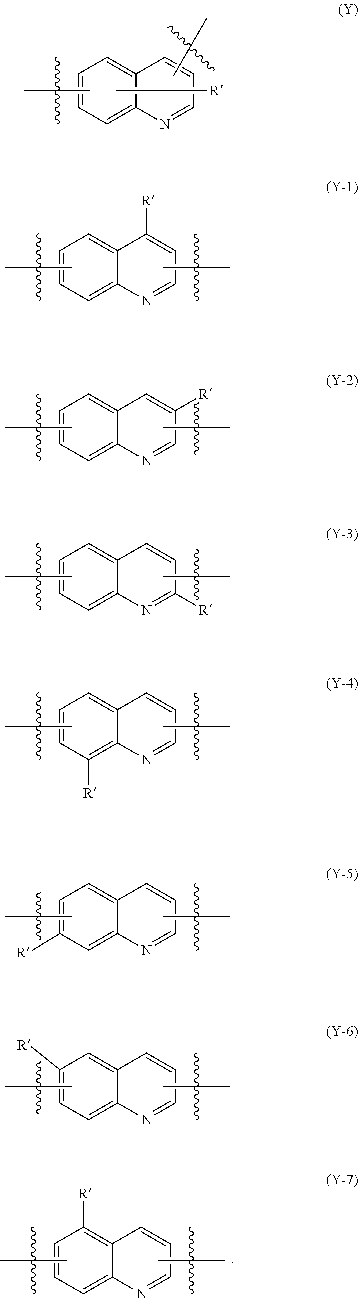

In some embodiments, the compound shown in Formula 1 is selected from structures shown in the following Formulae 1-1 to 1-4:

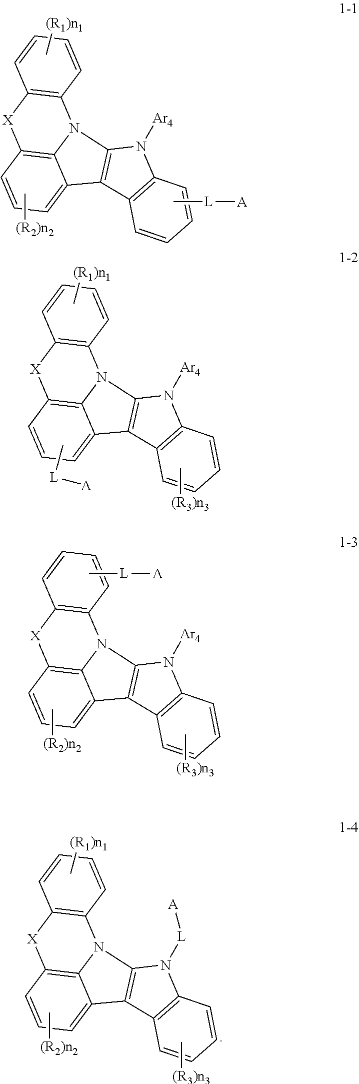

In some embodiments, the compound shown in Formula 1 is selected from structures shown in the following Formulae 2-1 to 2-8:

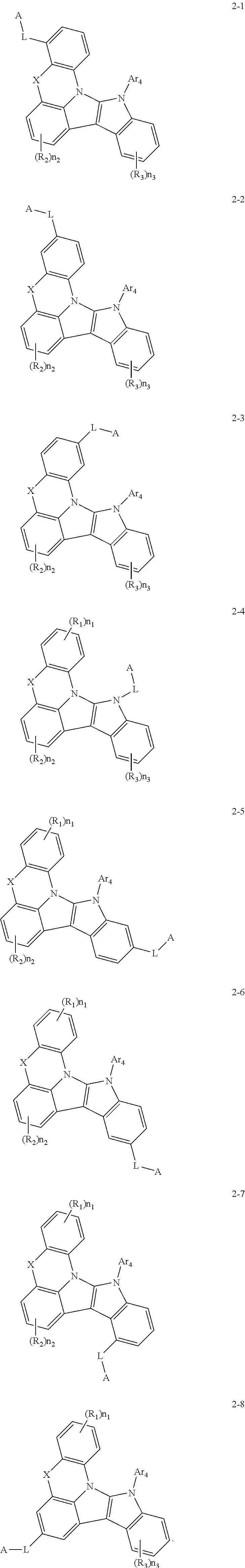

When the group [symbol] in the compound of the present disclosure is linked in ways as shown in Formulae 2-1 to 2-7, the core structure exhibits high stability and improved molecular thermal stability; and the compound, when used in organic luminescent layer of a device, can prolong the service life of the device.

In the present disclosure, Het is a nitrogen-containing heteroarylene having 3 to 20 carbon atoms group. Optionally, Het is a nitrogen-containing heteroarylene group having 3, 4, 5, 6, 7, 8, 9, or 10 carbon atoms. Preferably, the Het group contains at least two nitrogen atoms.

In some embodiments, Het is selected from triazinylene, pyrimidinylidene, or pyridylidene.

In some embodiments, Het is selected from

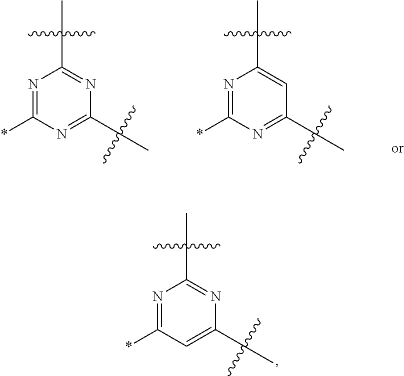

wherein [symbol] represents a bond linked with L, and [symbol] represents bond linked with L₁ or L₂.

In some embodiments of the present disclosure, Het or HAr is an electron-deficient nitrogen-containing heteroaryl group. The electron-deficient nitrogen-containing heteroaryl group contains at least one nitrogen atom. The sp² hybridized nitrogen atom can, on the whole, reduce, rather than increasing, the electron cloud density in the conjugated system of the heteroaryl group. The lone pair electrons on the heteroatom do not participate in the conjugated system, and the heteroatom, due to its strong electronegativity, reduces the electron cloud density in the conjugated system. For example, electron-deficient nitrogen-containing heteroaryl groups may include, but are not limited to, triazinyl, pyrimidyl, quinolyl, quinoxalinyl, quinazolinyl, isoquinolyl, benzimidazolyl, benzothiazolyl, benzoxazolyl, phenanthrolinyl, benzoquinazolinyl, phenanthroimidazolyl, benzofuropyrimidyl, benzothienopyrimidyl, etc. The electron-deficient nitrogen-containing heteroaryl group can form an electron-transport core group of the compound, so that effective electron transport can be achieved in the compound, and further the mobilities of electrons and holes in an organic luminescent layer can be effectively balanced.

In other embodiments of the present disclosure, HAr is an electron-rich aromatic group. The group on the whole has a high electron cloud density. For example, electron-rich aromatic groups may include, but are not limited to, phenylene, naphthylene, biphenylene, anthrylene, phenanthrylene, fluorenylidene, dibenzothienylene, dibenzofuranylene, carbazolylene, triphenylene, pyrenylene, perylenylene, spirobifluorenylidene, etc. The electron-rich aromatic group can form a hole-transport auxiliary group of the compound, so that effective hole transport can be achieved in the compound, and further the mobilities of electrons and holes in an organic luminescent layer can be effectively balanced.

In some embodiments, HAr is selected from substituted or unsubstituted arylene having 6, 7, 8, 9, 10, 11, 13, 14, 15, 16, 17, 18, 19, 20, 21, 22, 23, 24, or 25 carbon atoms, and substituted or unsubstituted heteroarylene having 5, 6, 7, 8, 9, 10, 11, 12, 13, 14, 15, 16, 17, or 18 carbon atoms. Optionally, substituents in HAr are identical or different, and are each independently selected from deuterium, cyano, halogen, alkyl having 1 to 4 carbon atoms, deuterated alkyl having 1 to 4 carbon atoms, and aryl having 6 to 12 carbon atoms.

In some embodiments, HAr is selected from substituted or unsubstituted phenylene, substituted or unsubstituted naphthylene, substituted or unsubstituted biphenylene, substituted or unsubstituted anthrylene, substituted or unsubstituted phenanthrylene, substituted or unsubstituted fluorenylidene, substituted or unsubstituted spirobifluorenylidene, substituted or unsubstituted dibenzothienylene, substituted or unsubstituted dibenzofuranylene, and substituted or unsubstituted carbazolylene, or selected from the following substituted or unsubstituted groups:

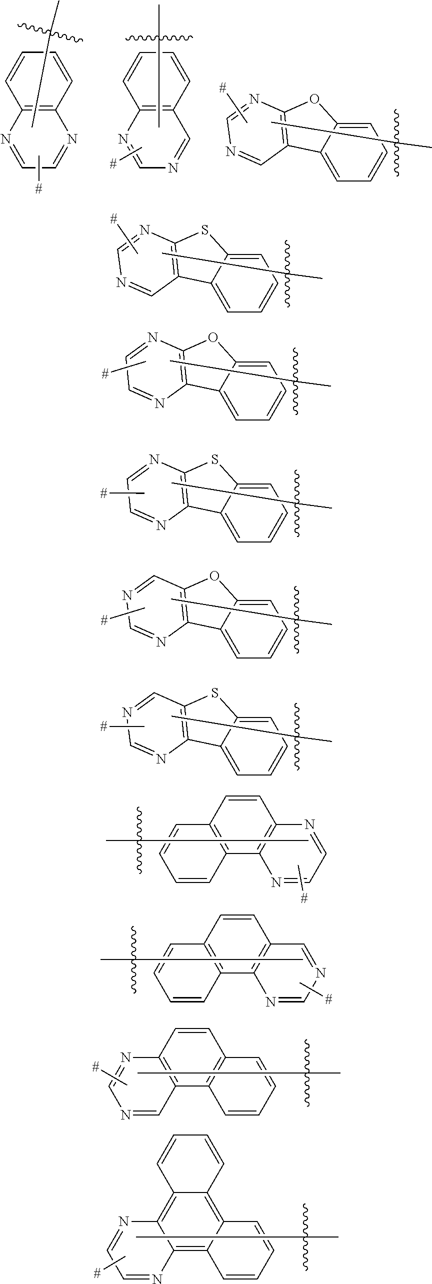

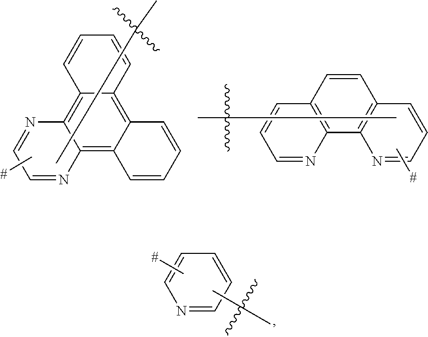

wherein [symbol] represents bond linked with L, and [symbol] represents bond linked with L₃; each substituent in HAr is identical or different, and is independently selected from deuterium, fluorine, cyano, trideuteromethyl, trifluoromethyl, alkyl having 1 to 4 carbon atoms, and phenyl.

In some embodiments, HAr is selected from substituted or unsubstituted group W, the unsubstituted group W is selected from the following groups:

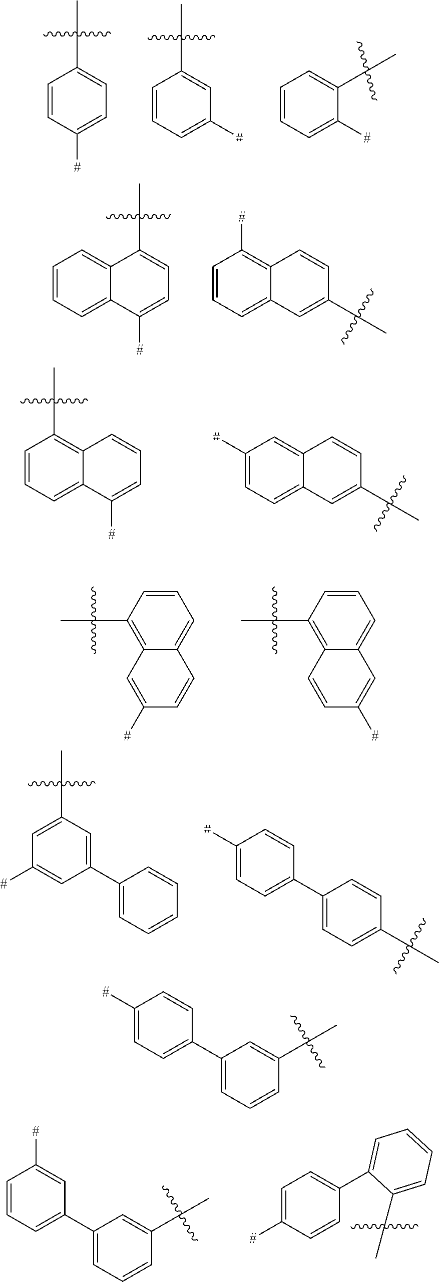

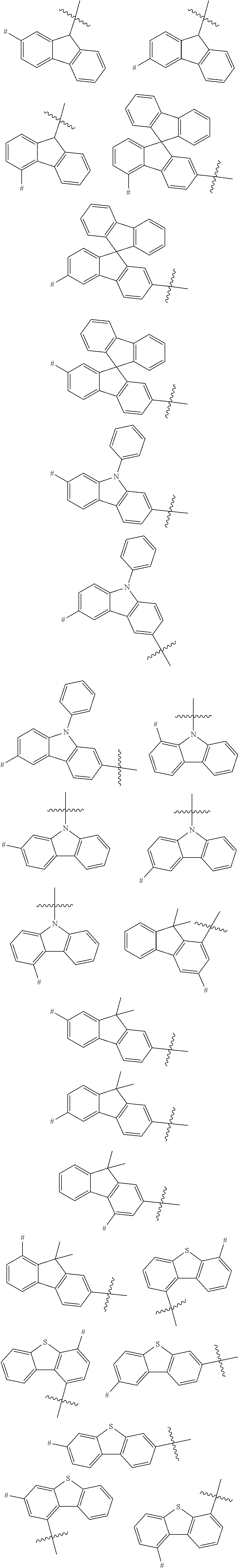

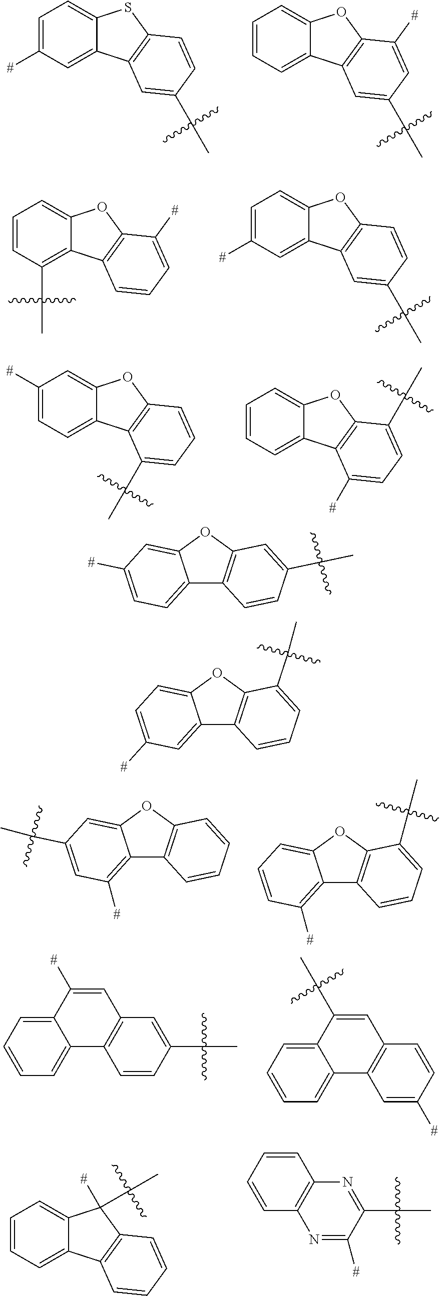

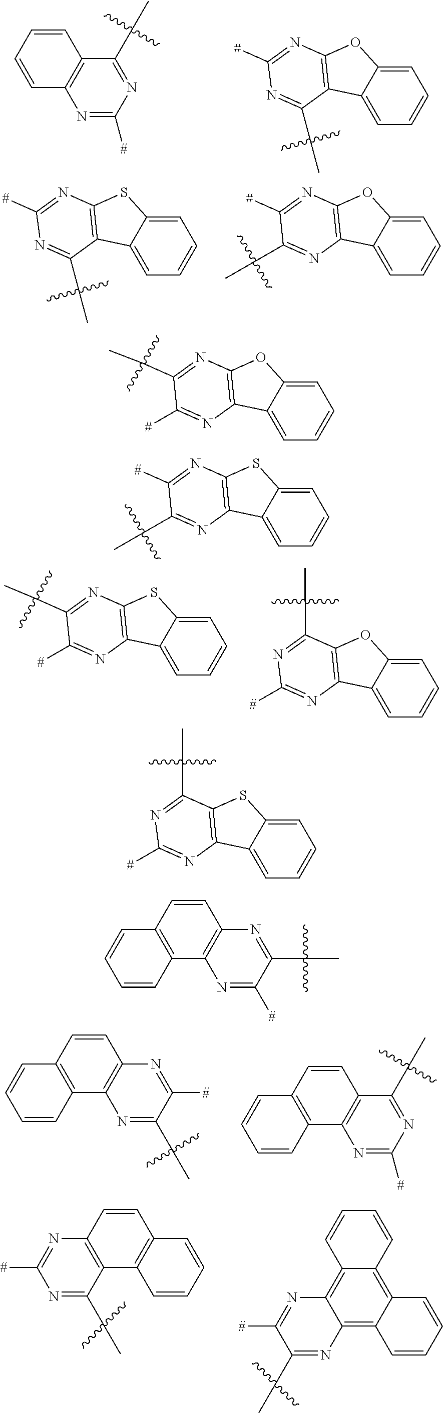

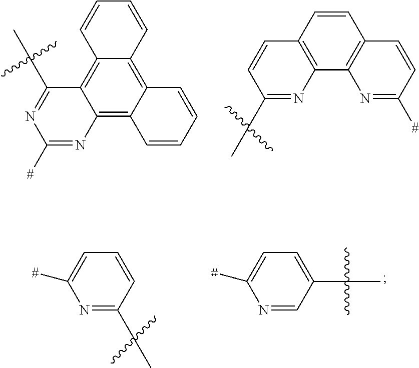

wherein [symbol] represents bond linked with L, and [symbol] represents bond linked with L₃;
the substituted group W is the group formed by the unsubstituted group W being substituted by one or more substituents, each substituent is independently selected from the group consisting of deuterium, fluorine, cyano, trideuteromethyl, trifluoromethyl, methyl, ethyl, isopropyl, tert-butyl, and phenyl; and when the number of the substituents is greater than 1, the substituents are identical or different.

In some embodiments, Ar₁ and Ar₂ are identical or different, and are each independently selected from the group consisting of substituted or unsubstituted aryl having 6 to 25 carbon atoms, and substituted or unsubstituted heteroaryl having 5 to 20 carbon atoms.

In some embodiments, Ar₁ and Ar₂ are each independently selected from the group consisting of substituted or unsubstituted aryl having 6, 7, 8, 9, 10, 11, 12, 13, 14, 15, 16, 17, 18, 19, 20, 21, 22, 23, 24, or 25 carbon atoms, and substituted or unsubstituted heteroaryl having 5, 6, 7, 8, 9, 10, 12, 13, 14, 15, 16, 17, 18, 19, or 20 carbon atoms.

In some embodiments, Ar₃ is selected from the group consisting of hydrogen, substituted or unsubstituted aryl having 6 to 25 carbon atoms, and substituted or unsubstituted heteroaryl having 5 to 20 carbon atoms.

In some embodiments, Ar₃ is selected from the group consisting of hydrogen, substituted or unsubstituted aryl having 6, 7, 8, 9, 10, 11, 12, 13, 14, 15, 16, 17, 18, 19, 20, 21, 22, 23, 24, or 25 carbon atoms, and substituted or unsubstituted heteroaryl having 5, 6, 7, 8, 9, 10, 12, 13, 14, 15, 16, 17, 18, 19, or 20 carbon atoms.

In some embodiments, Ar₄ is selected from the group consisting of single bond, substituted or unsubstituted aryl having 6 to 25 carbon atoms, and substituted or unsubstituted heteroaryl having 5 to 20 carbon atoms.

In some embodiments, Ar₄ is selected from the group consisting of single bond, substituted or unsubstituted aryl having 6, 7, 8, 9, 10, 11, 12, 13, 14, 15, 16, 17, 18, 19, 20, 21, 22, 23, 24, or 25 carbon atoms, and substituted or unsubstituted heteroaryl having 5, 6, 7, 8, 9, 10, 12, 13, 14, 15, 16, 17, 18, 19, or 20 carbon atoms.

In some embodiments, substituents in Ar₁, Ar₂, Ar₃, and Ar₄ are each independently selected from the group consisting of deuterium, halogen, cyano, haloalkyl having 1 to 4 carbon atoms, deuterated alkyl having 1 to 4 carbon atoms, alkyl having 1 to 4 carbon atoms, cycloalkyl having 5 to 10 atom groups, aryl having 6 to 12 carbon atoms, heteroaryl having 5 to 12 carbon atoms, and trialkylsilyl having 3 to 8 atom groups; and optionally, any two adjacent substituents form a benzene ring or a fluorene ring.

In some embodiments, Ar₁ and Ar₂ are identical or different, and are each independently selected from the group consisting of substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted spirobifluorenyl, substituted or unsubstituted triphenylene, substituted or unsubstituted pyrenyl, substituted or unsubstituted perylenyl, substituted or unsubstituted pyridyl, substituted or unsubstituted dibenzothienyl, substituted or unsubstituted dibenzofuranyl, substituted or unsubstituted carbazolyl, substituted or unsubstituted quinolyl, substituted or unsubstituted phenanthrolin, substituted or unsubstituted benzothiazolyl, substituted or unsubstituted benzoxazolyl, and substituted or unsubstituted benzimidazolyl.

Optionally, the substituents in Ar₁ and Ar₂ are each independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, pyridyl, dibenzofuranyl, dibenzothienyl, and carbazolyl; and optionally, in Ar₁ and Ar₂, any two adjacent substituents form a benzene ring.

In some embodiments, Ar₃ is selected from the group consisting of hydrogen, substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted spirobifluorenyl, substituted or unsubstituted triphenylene, substituted or unsubstituted pyrenyl, substituted or unsubstituted perylenyl, substituted or unsubstituted pyridyl, substituted or unsubstituted dibenzothienyl, substituted or unsubstituted dibenzofuranyl, substituted or unsubstituted carbazolyl, substituted or unsubstituted quinolyl.

Optionally, the substituents in Ar₃ are each independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, pyridyl, dibenzofuranyl, dibenzothienyl, carbazolyl, benzoxazolyl, and benzothiazolyl; and optionally, in Ar₃, any two adjacent substituents form a benzene ring.

In some embodiments, Ar₄ is selected from the group consisting of single bond, substituted or unsubstituted phenyl, substituted or unsubstituted biphenyl, substituted or unsubstituted triphenyl, substituted or unsubstituted naphthyl, substituted or unsubstituted anthryl, substituted or unsubstituted phenanthryl, substituted or unsubstituted fluorenyl, substituted or unsubstituted spirobifluorenyl, substituted or unsubstituted triphenylene, substituted or unsubstituted pyrenyl, substituted or unsubstituted perylenyl, substituted or unsubstituted pyridyl, substituted or unsubstituted dibenzothienyl, substituted or unsubstituted dibenzofuranyl, and substituted or unsubstituted carbazolyl.

Optionally, the substituents in Ar₄ are independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, dibenzothienyl, dibenzofuranyl, and carbazolyl; and optionally, in Ar₄, any two adjacent substituents form a benzene ring.

In some embodiments, Ar₁ and Ar₂ are each independently selected from substituted or unsubstituted groups T; Ar₃ is selected from hydrogen, and substituted or unsubstituted groups T; and Ar₄ is single bond or is selected from substituted or unsubstituted groups T, the unsubstituted group T is selected from the group consisting of the following groups:

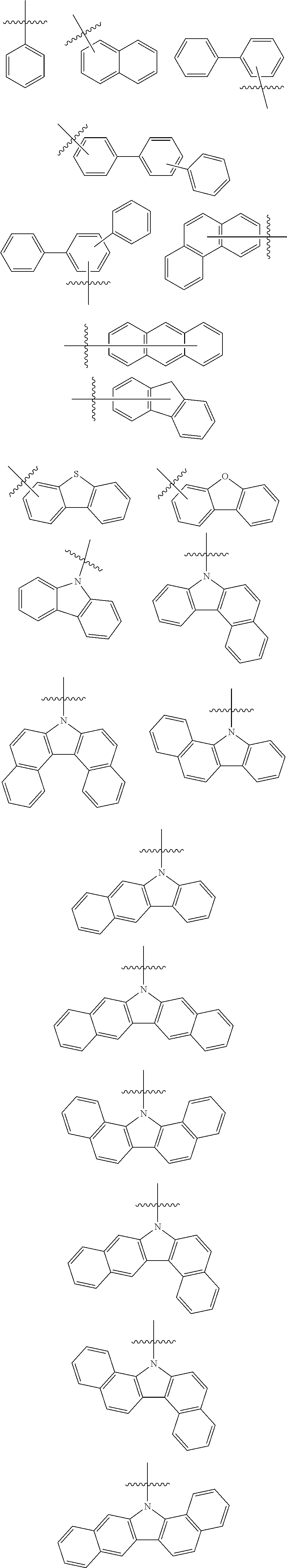

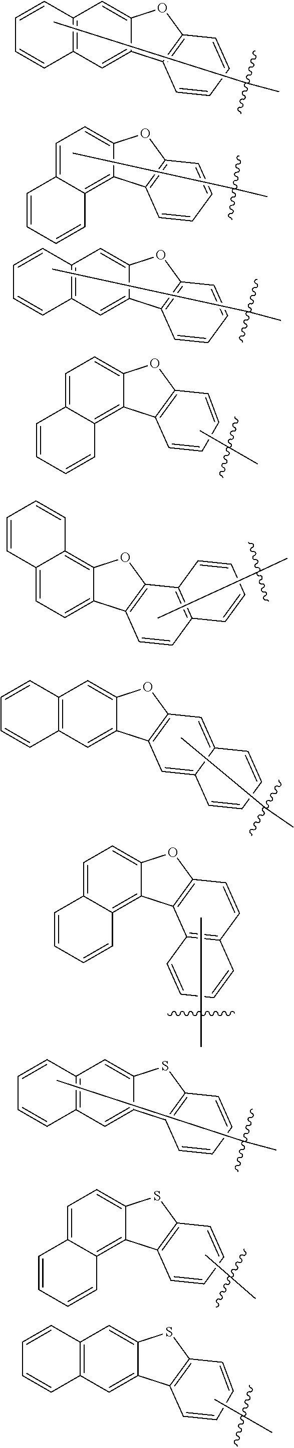

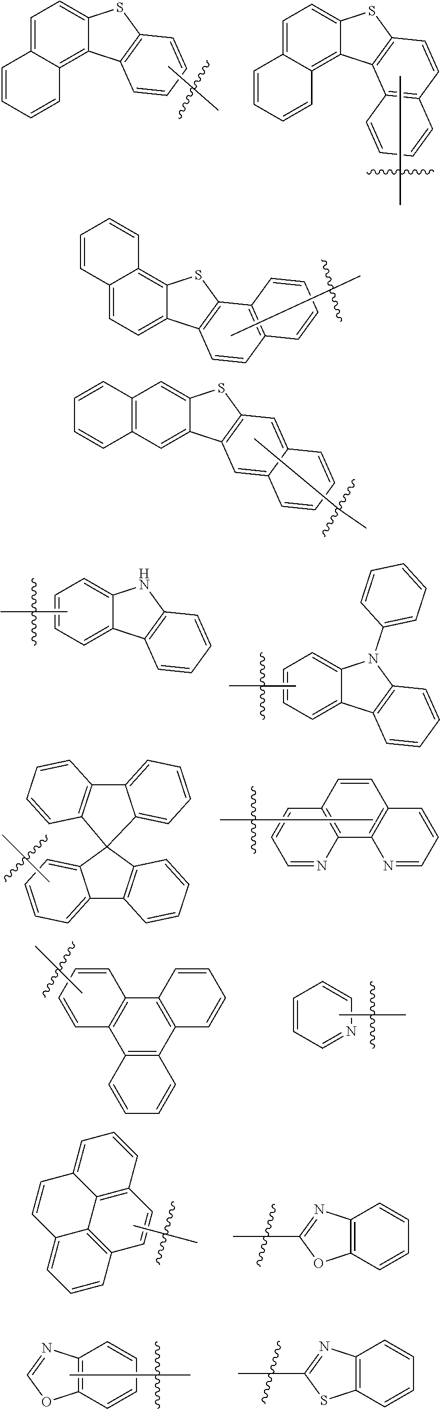

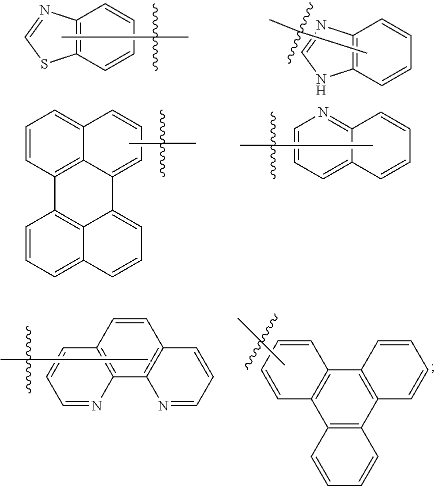

the substituted group T is a group formed by unsubstituted group T being substituted by one or more substituents, each substituent of the substituted group T is independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, pyridyl, dibenzofuranyl, dibenzothienyl, carbazolyl, benzoxazolyl, and benzothiazolyl; when the number of the substituents on a group T is greater than 1, the substituents are identical or different.

In some embodiments, Ar₁ and Ar₂ are identical or different, and are each independently selected from the following groups:

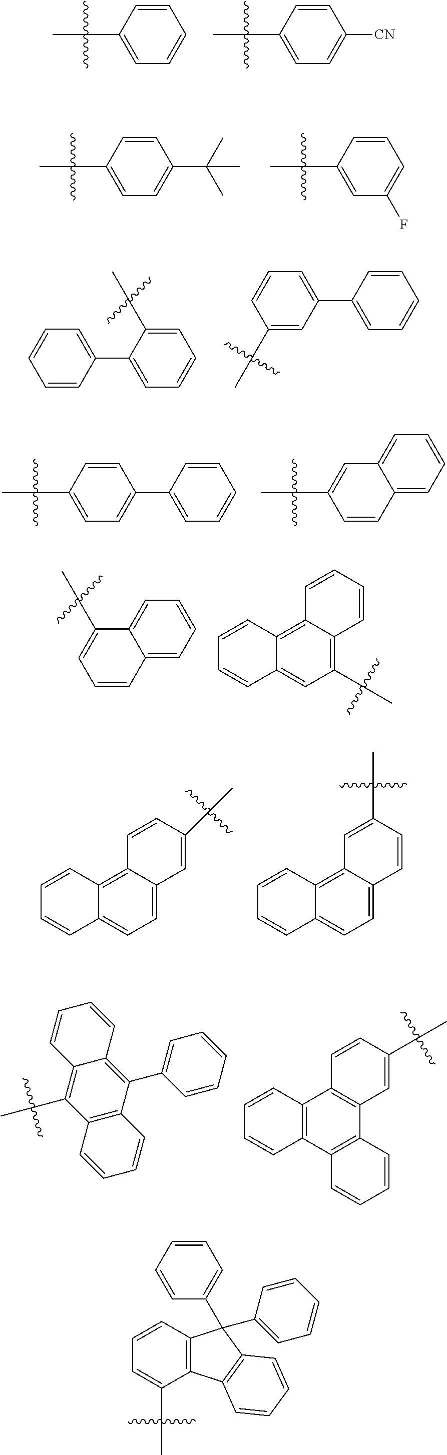

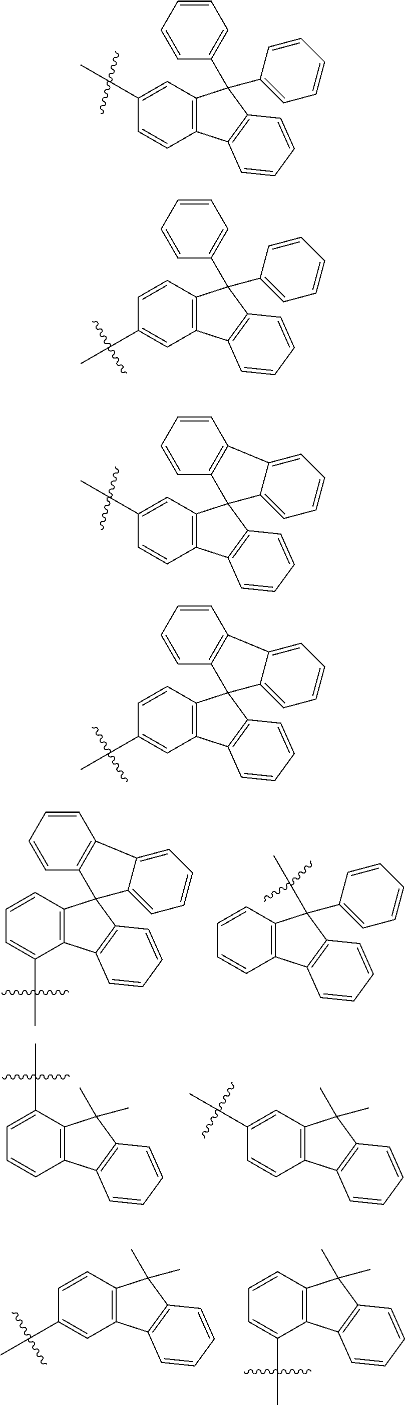

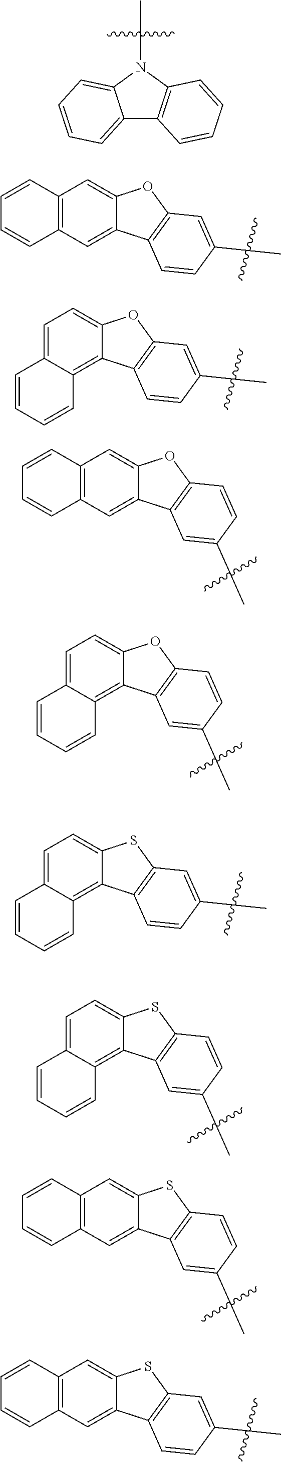

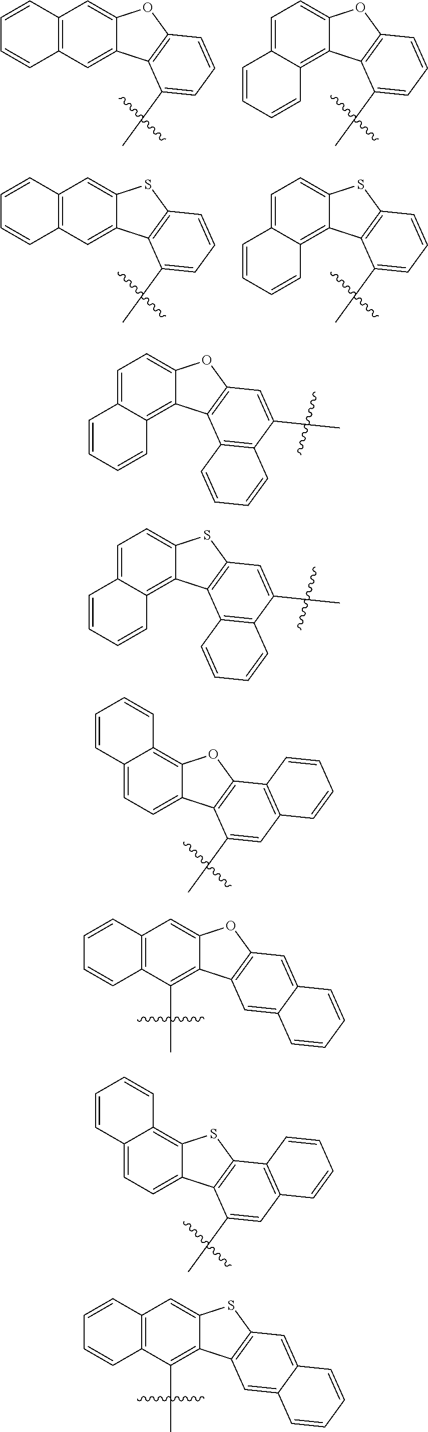

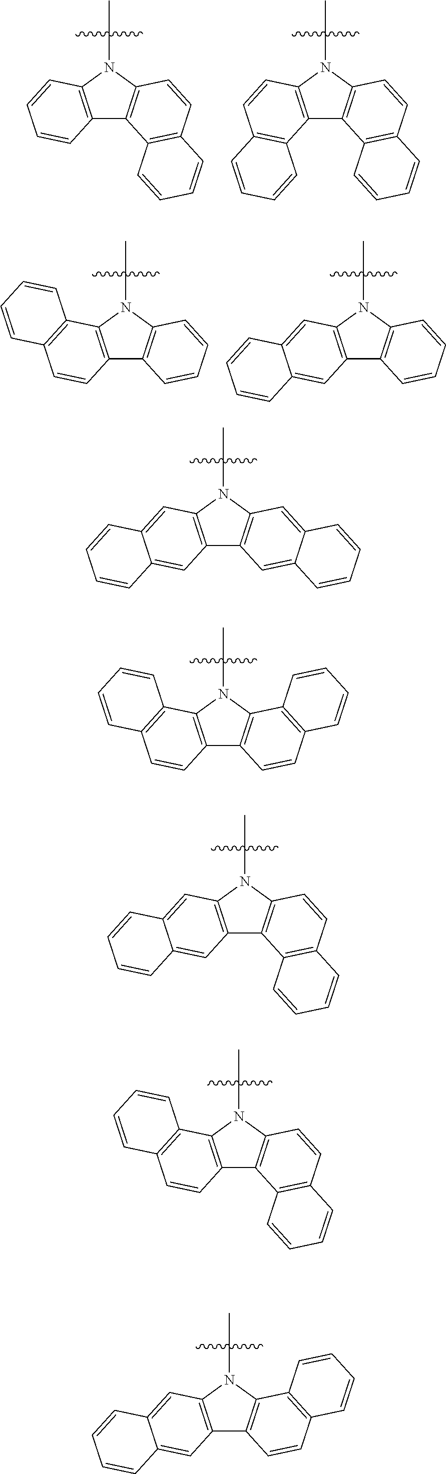

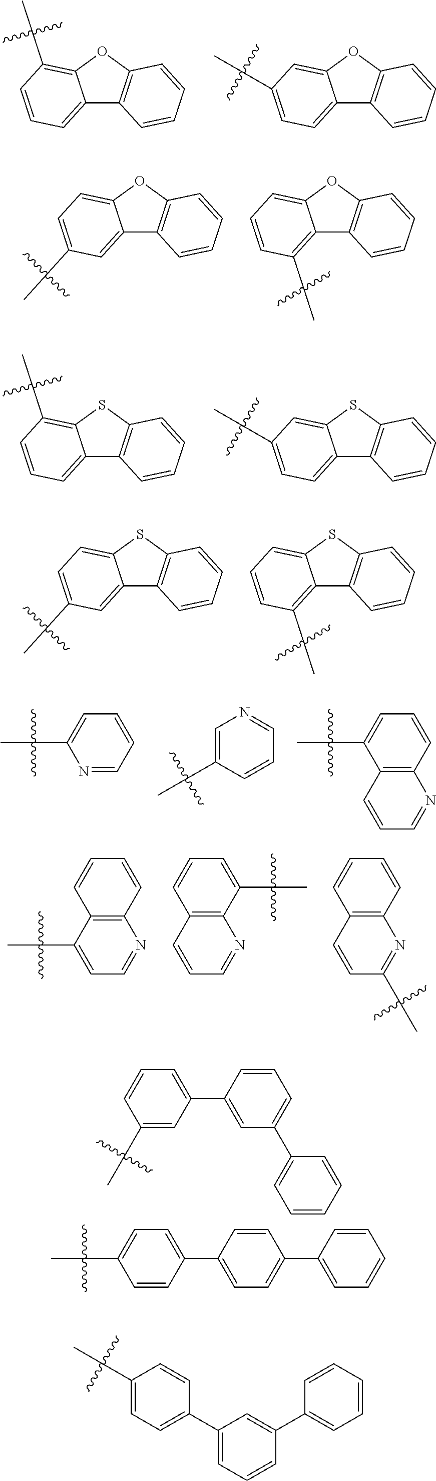

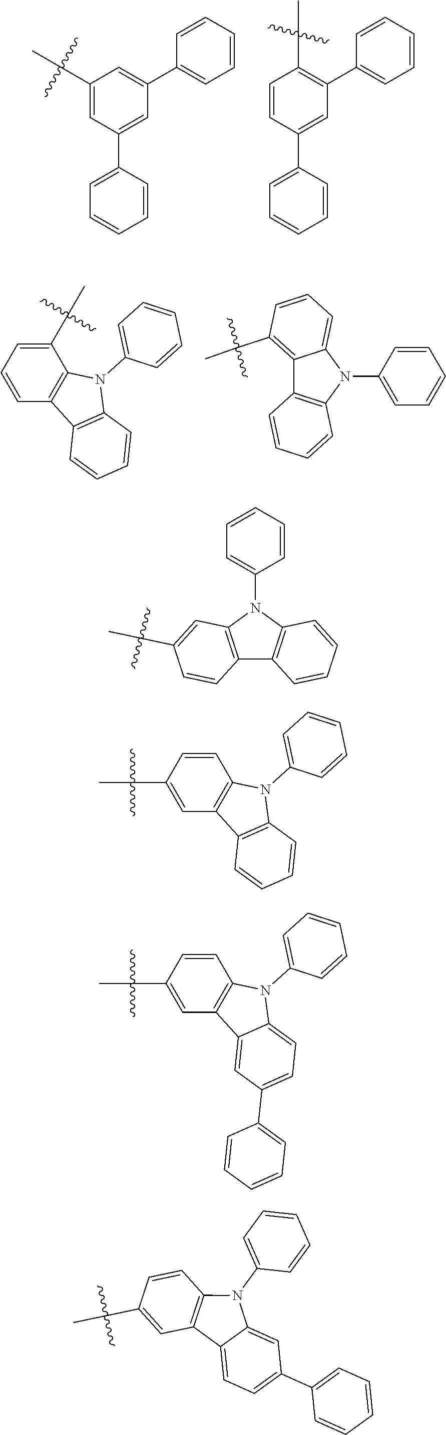

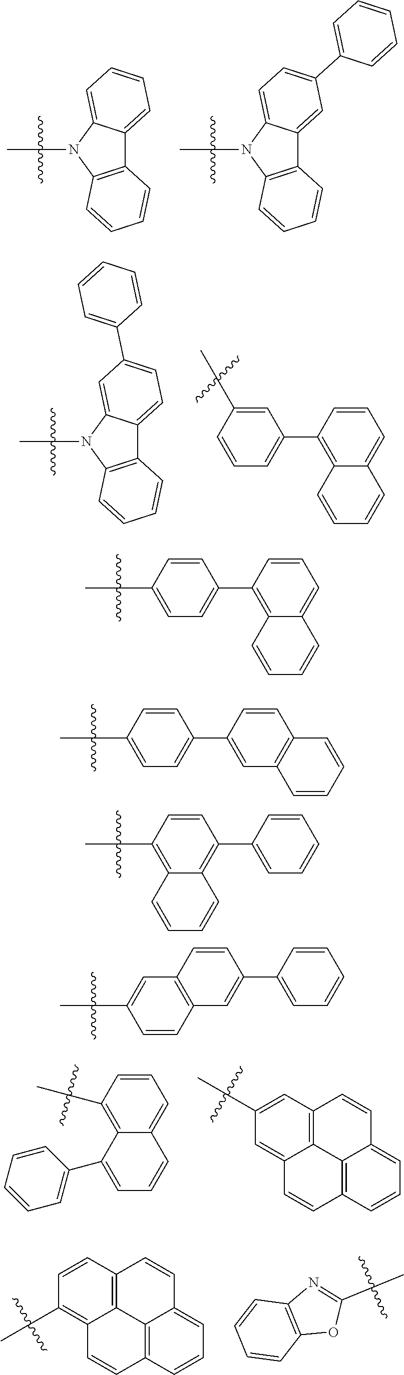

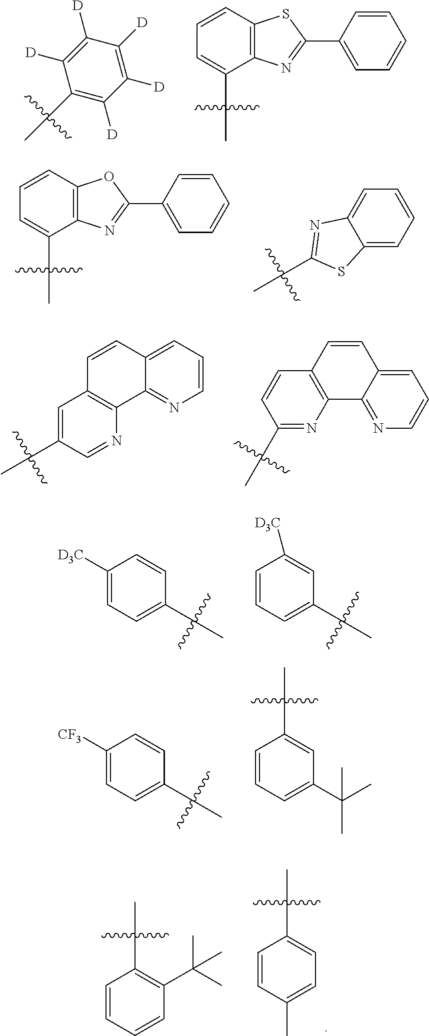

In some embodiments, Ar₃ is selected from hydrogen or the group consisting of the following groups:

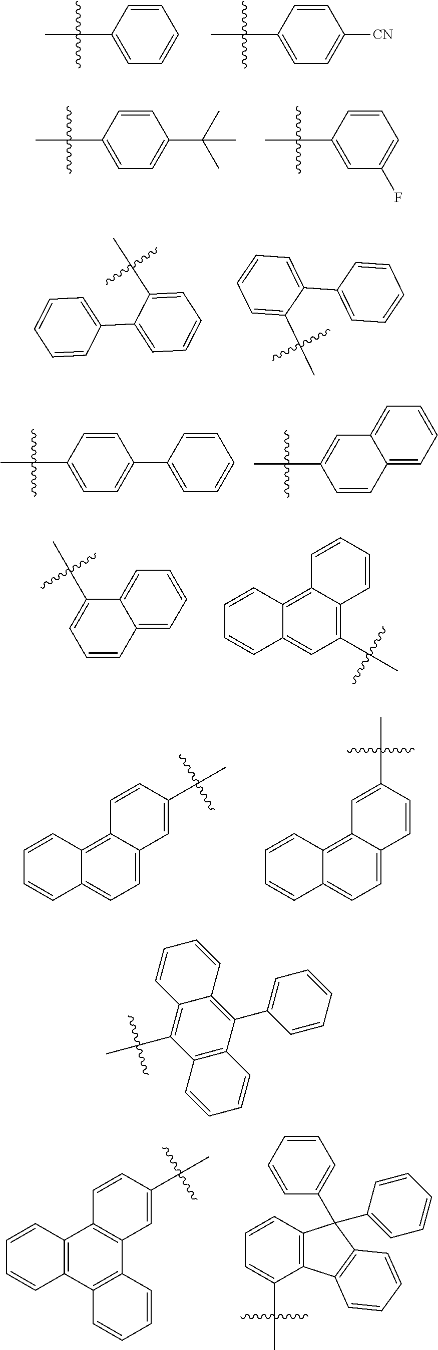

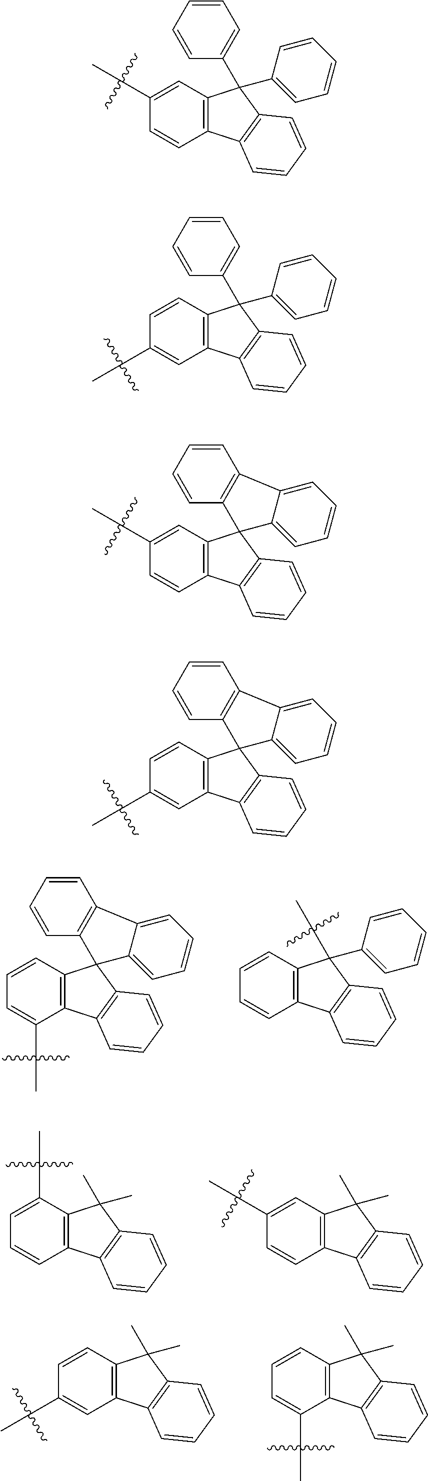

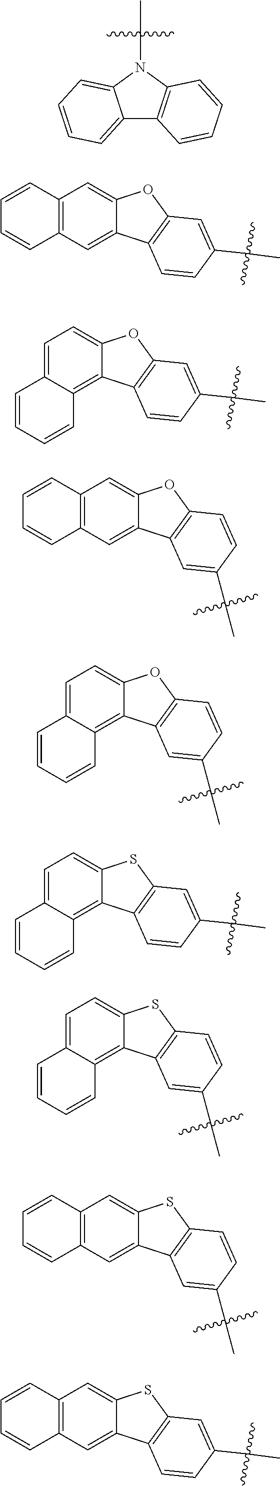

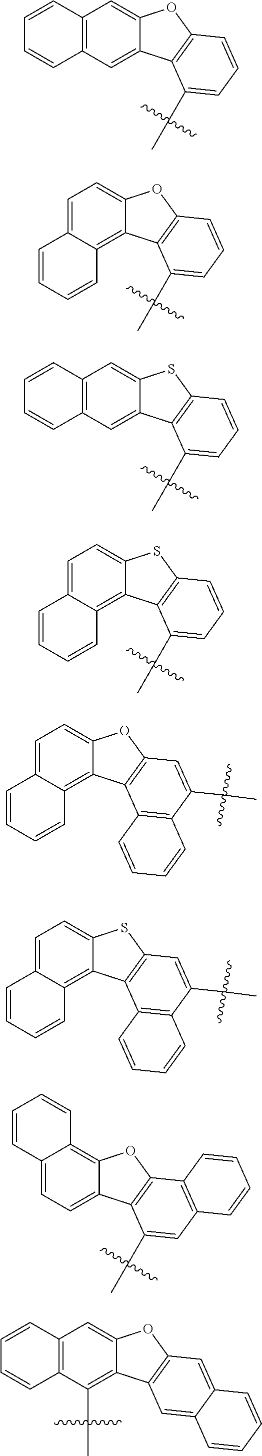

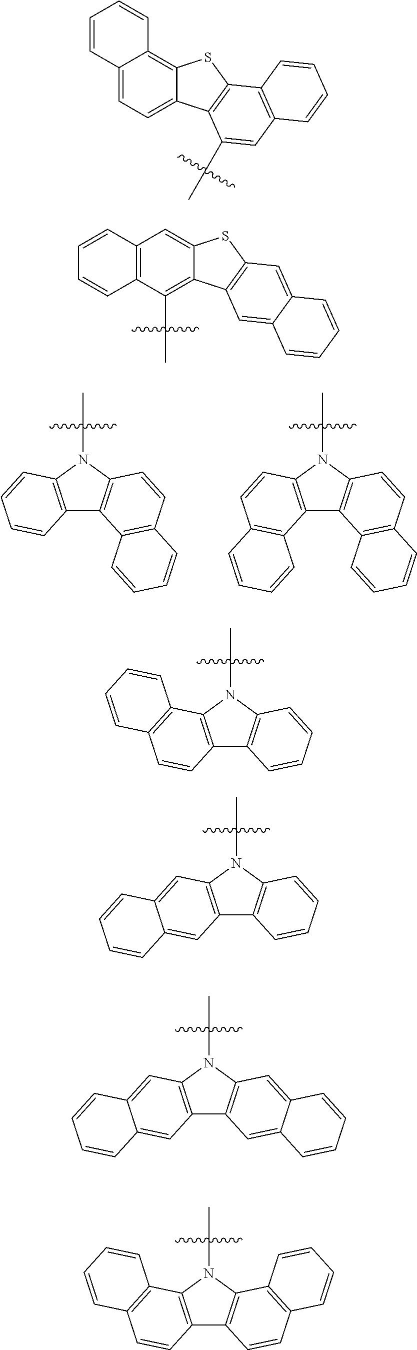

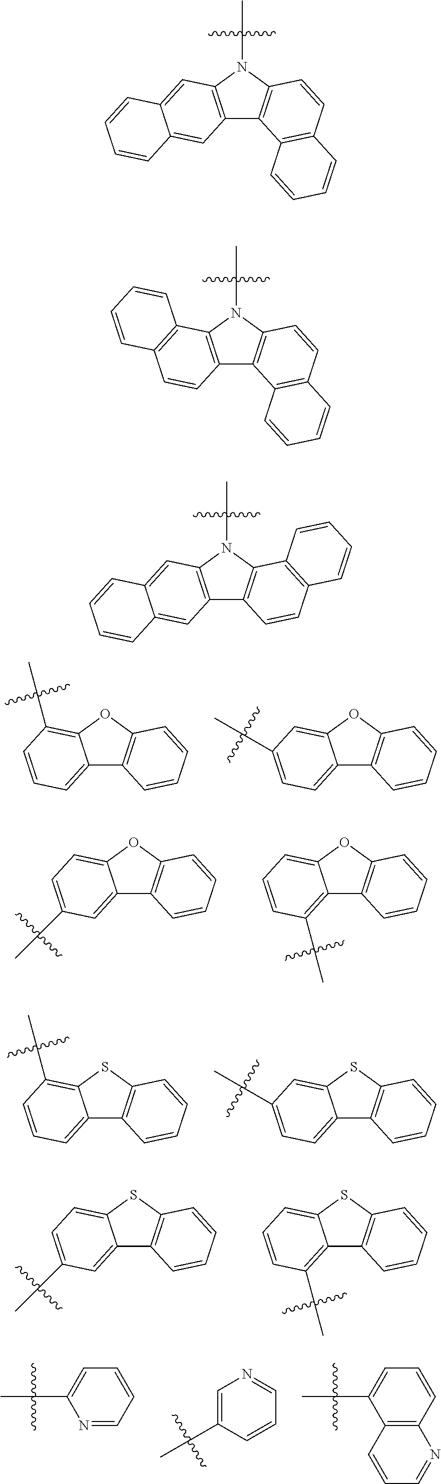

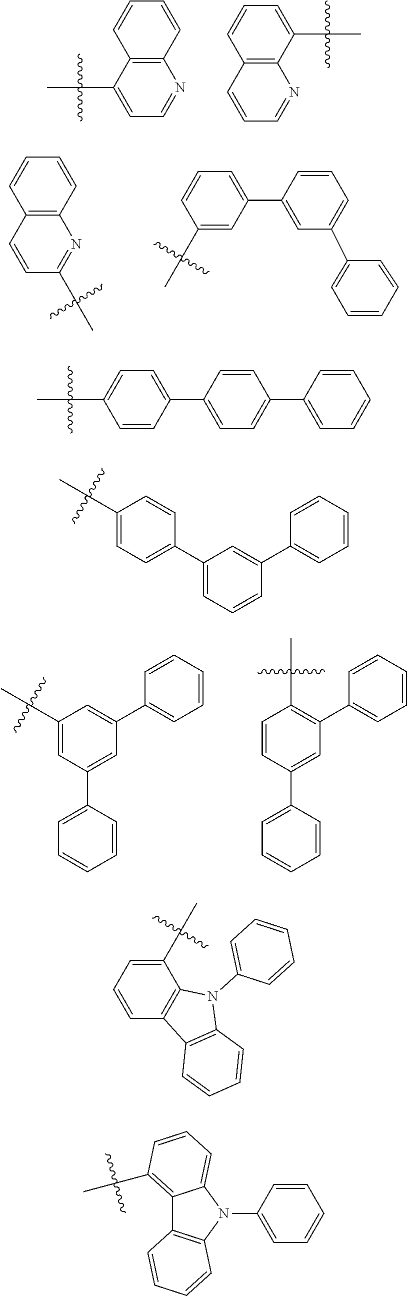

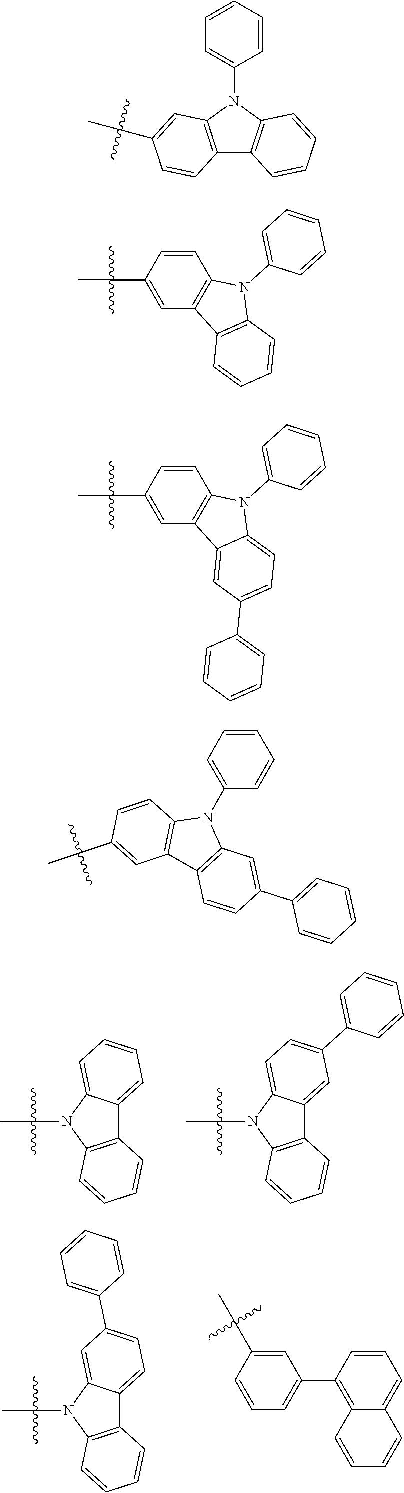

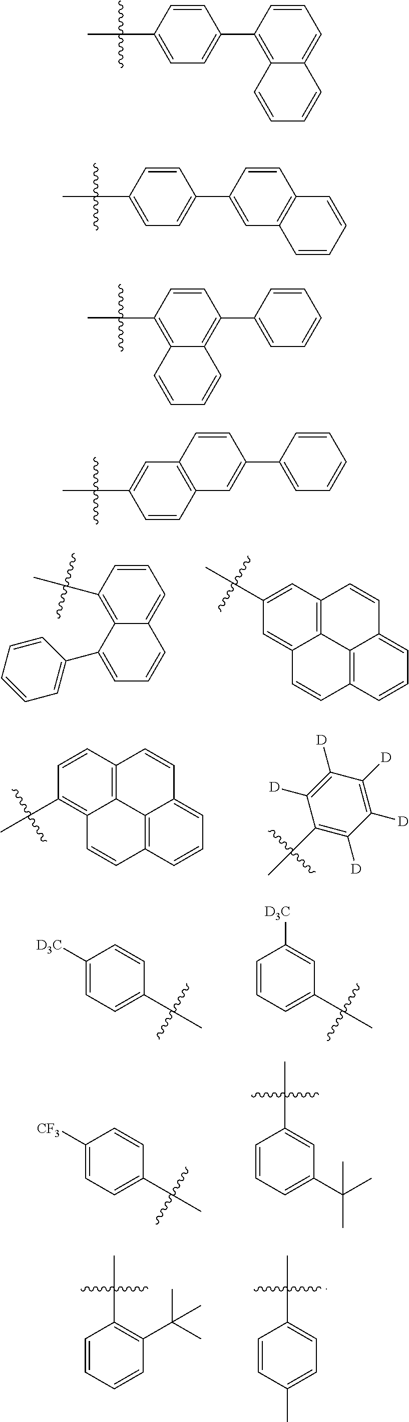

In some embodiments, Ar₄ is selected from single bond or the group consisting of the following groups:

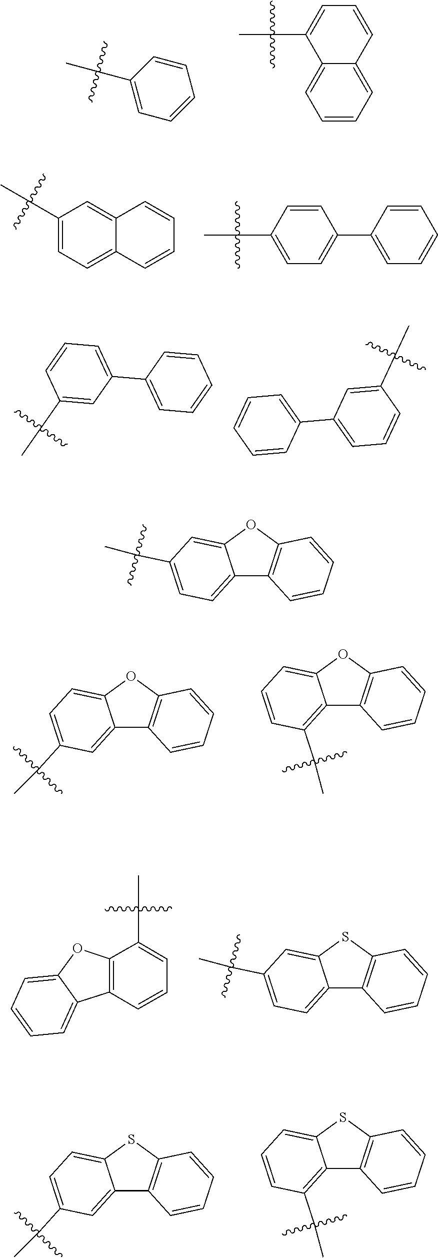

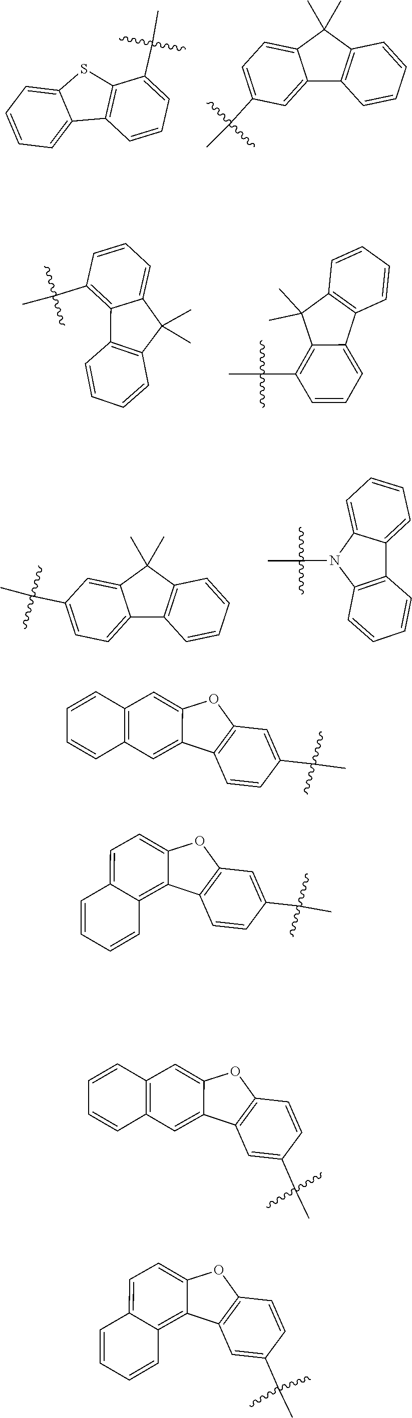

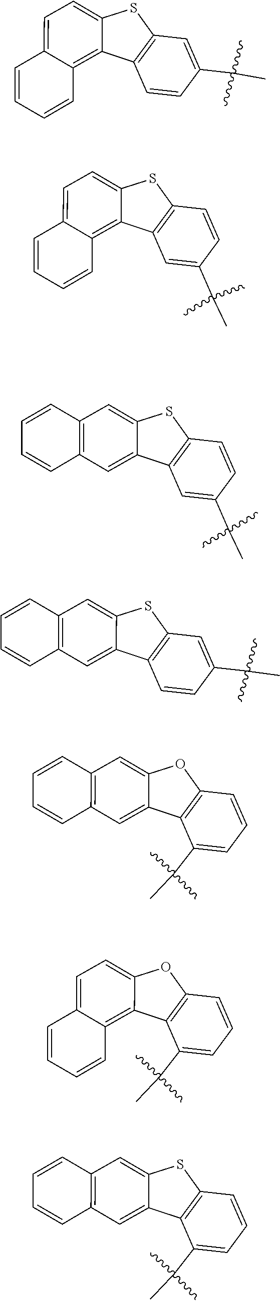

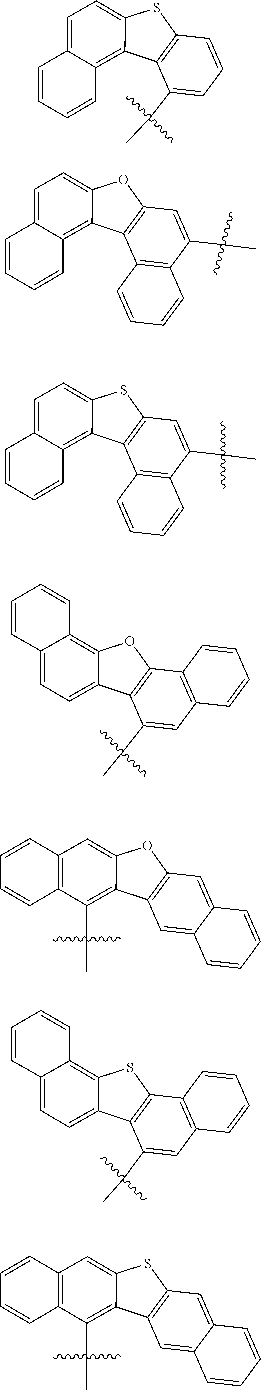

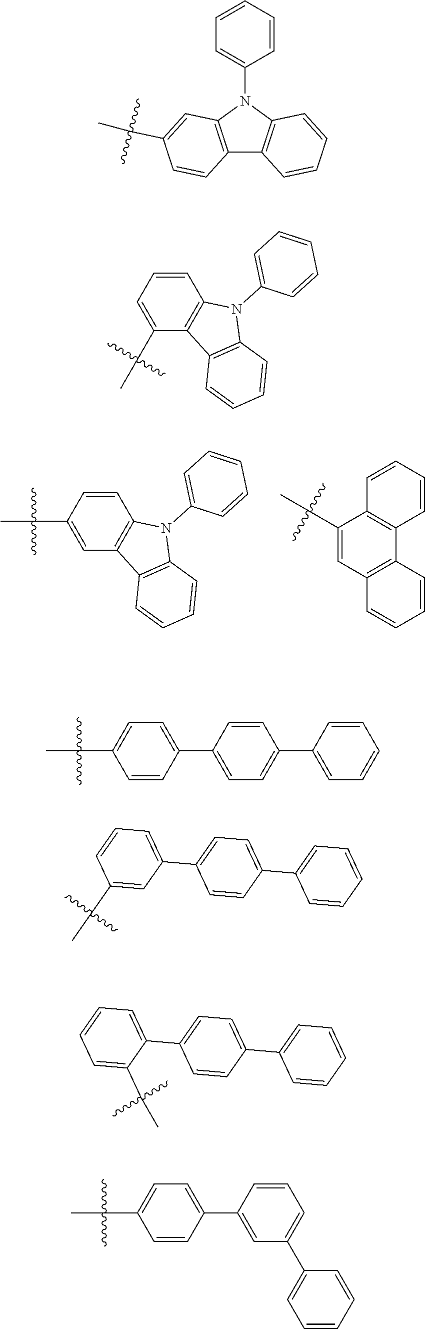

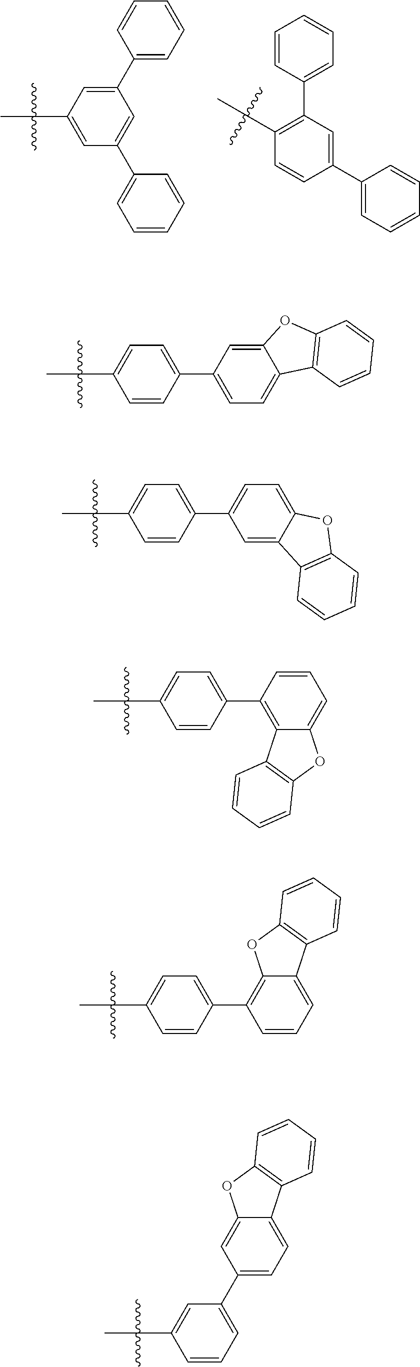

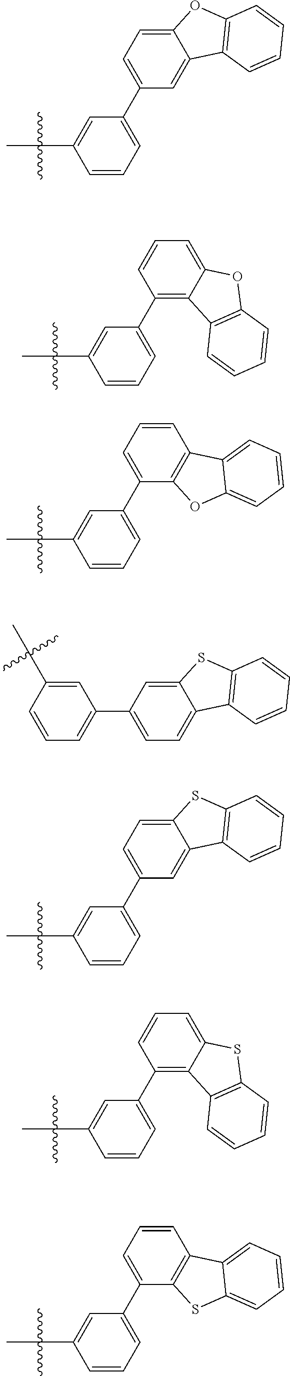

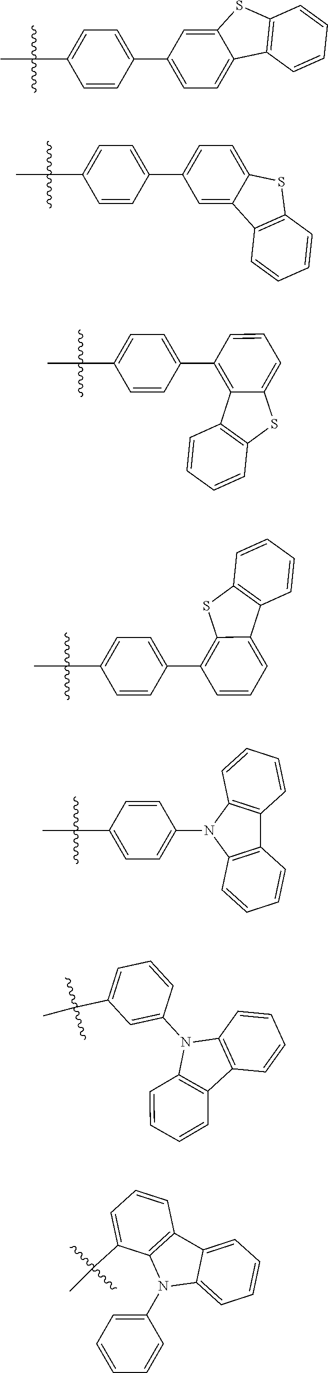

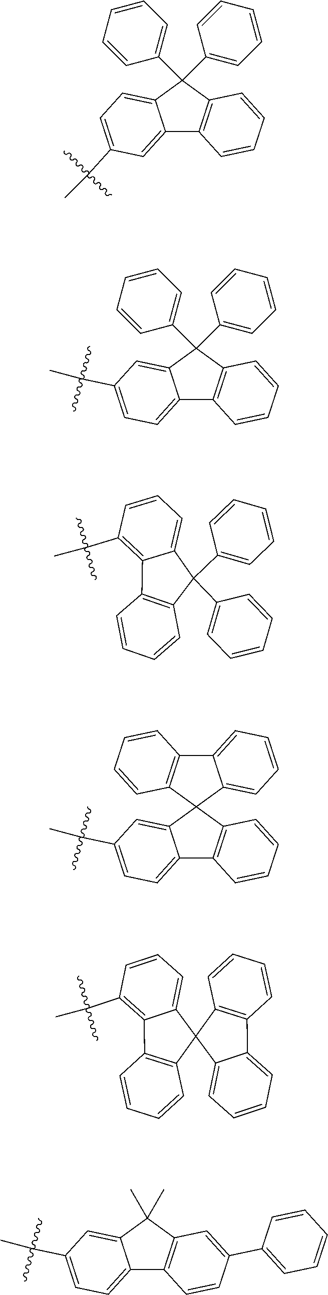 [SMILES: *c1ccc2c(c1)-c1ccccc1C2(c1ccccc1)c1ccccc1 ; *c1ccc2c(c1)C(c1ccccc1)(c1ccccc1)c1ccccc1-2 ; *c1cccc2c1-c1ccccc1C2(c1ccccc1)c1ccccc1 ; *c1ccc2c(c1)C1(c3ccccc3-c3ccccc31)c1ccccc1-2 ; *c1cccc2c1-c1ccccc1C21c2ccccc2-c2ccccc21 ; *c1ccc2c(c1)C(C)(C)c1cc(-c3ccccc3)ccc1-2]

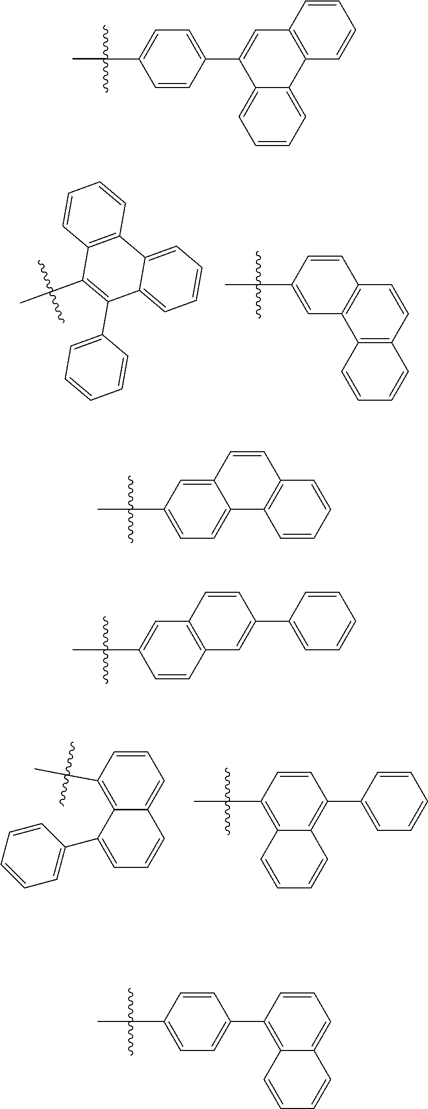

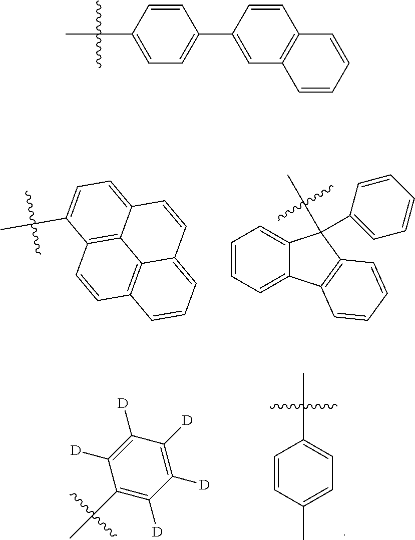

In some embodiments, L, L₁, L₂, and L₃ are identical or different, and are each independently selected from single bond, substituted or unsubstituted arylene having 6, 7, 8, 9, 10, 11, 12, 13, 14, or 15 carbon atoms, and substituted or unsubstituted heteroarylene having 5, 6, 7, 8, 9, 10, 11, 12, 13, 14, 15, 16, 17, or 18 carbon atoms.

Optionally, the substituents in L, L₁, L₂, and L₃ are each independently selected from the group consisting of deuterium, fluorine, cyano, alkyl having 1 to 5 carbon atoms, trialkylsilyl having 3 to 8 atom groups, fluoroalkyl having 1 to 4 carbon atoms, deuterated alkyl having 1 to 4 carbon atoms, phenyl, and naphthyl.

In some embodiments, L and L₃ are identical or different, and are each independently selected from the group consisting of single bond, substituted or unsubstituted phenylene, substituted or unsubstituted naphthylene, and substituted or unsubstituted biphenylene.

In some embodiments, L₁ and L₂ are identical or different, and are each independently selected from the group consisting of single bond, substituted or unsubstituted phenylene, substituted or unsubstituted naphthylene, substituted or unsubstituted biphenylene, substituted or unsubstituted fluorenylidene, substituted or unsubstituted phenanthrylene, substituted or unsubstituted dibenzothienylene, substituted or unsubstituted dibenzofuranylene, substituted or unsubstituted carbazolylene, and substituted or unsubstituted pyridylidene.

Optionally, the substituents in L, L₃, L₁, and L₂ are each independently selected from the group consisting of deuterium, fluorine, cyano, methyl, ethyl, isopropyl, tert-butyl, trifluoromethyl, trideuteromethyl, trimethylsilyl, and phenyl.

In some embodiments, L and L₃ are each independently selected from single bond or the group consisting of the following groups:

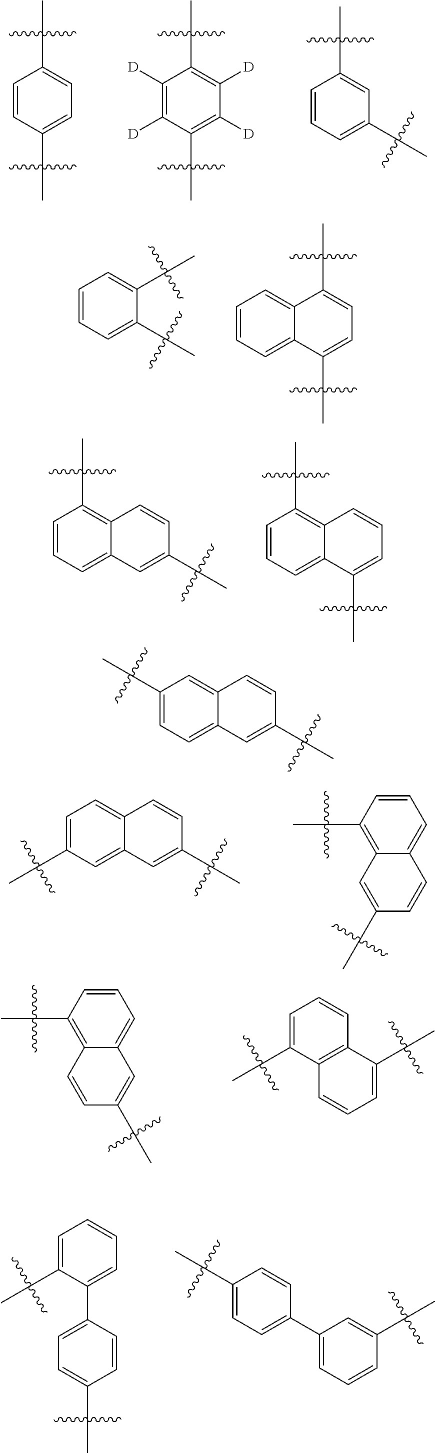

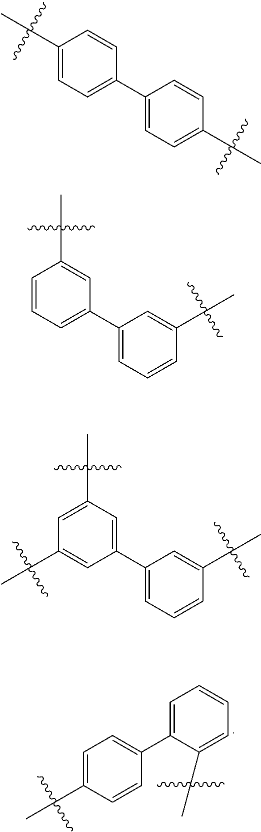 [SMILES: *c1ccc(-c2ccc(*)cc2)cc1 ; *c1cccc(-c2cccc(*)c2)c1 ; *c1cccc(-c2cc(*)cc(*)c2)c1 ; *c1ccc(-c2ccccc2*)cc1]

In some embodiments, L₁ and L₂ are independently selected from single bond or the group consisting of the following groups:

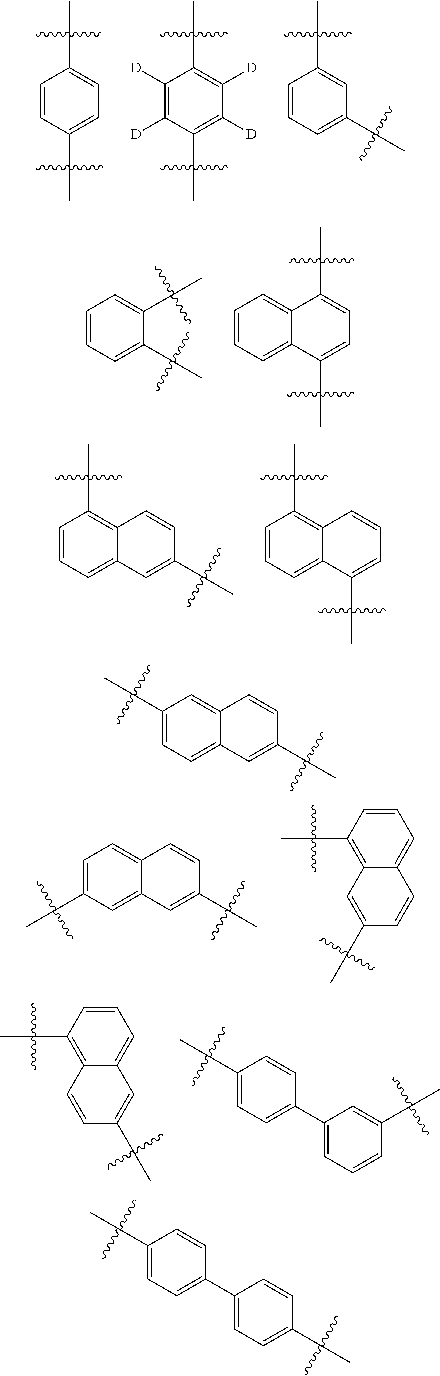

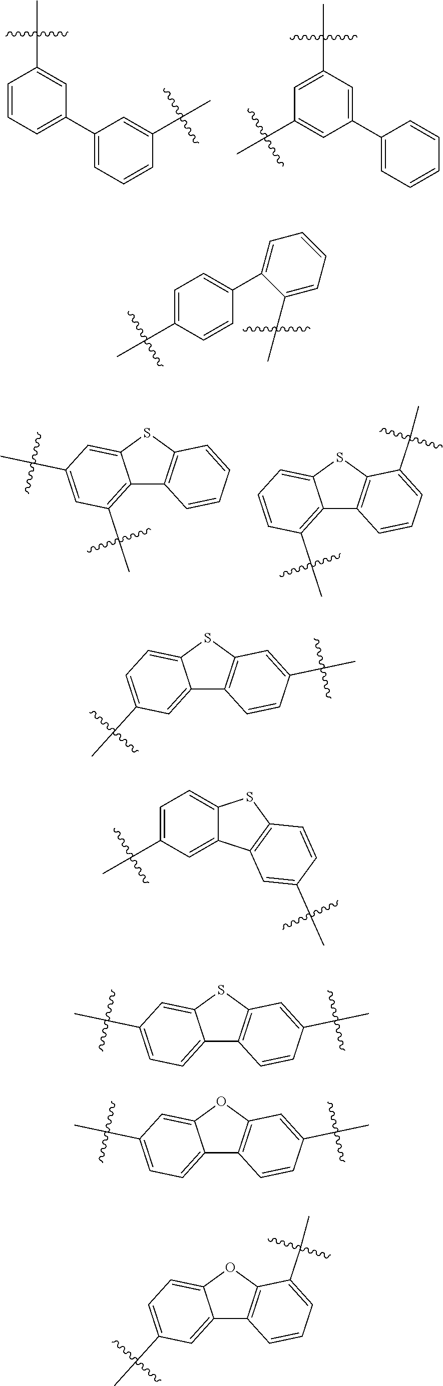

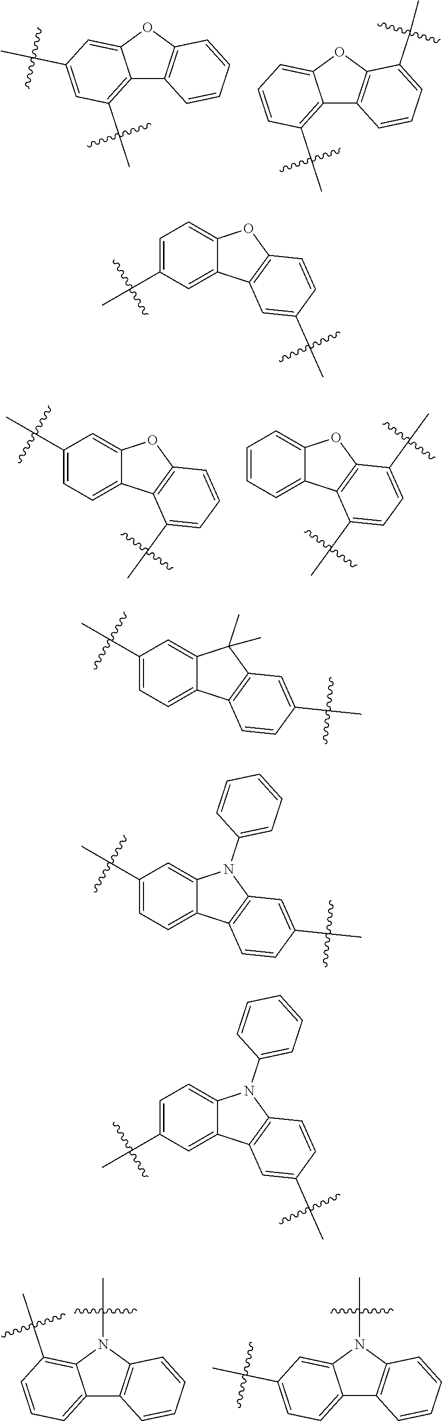

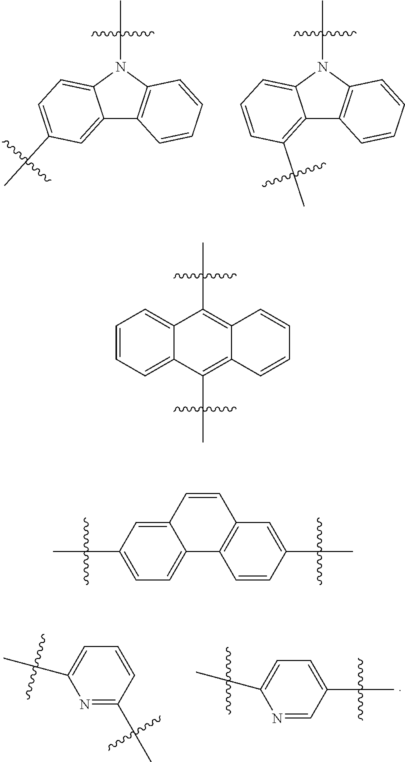

In some embodiments, each R₁, each R₂, and each R₃ is identical or different, and is independently selected from the group consisting of deuterium, cyano, fluorine, trimethylsilyl, trideuteromethyl, trifluoromethyl, cyclopentyl, cyclohexyl, methyl, ethyl, isopropyl, tert-butyl, phenyl, and naphthyl; and optionally, any two adjacent R₁ and/or any two adjacent R₂ and/or any two adjacent R₃ form a benzene ring.

In some embodiments,

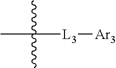

is selected from hydrogen or the group consisting of the following groups:

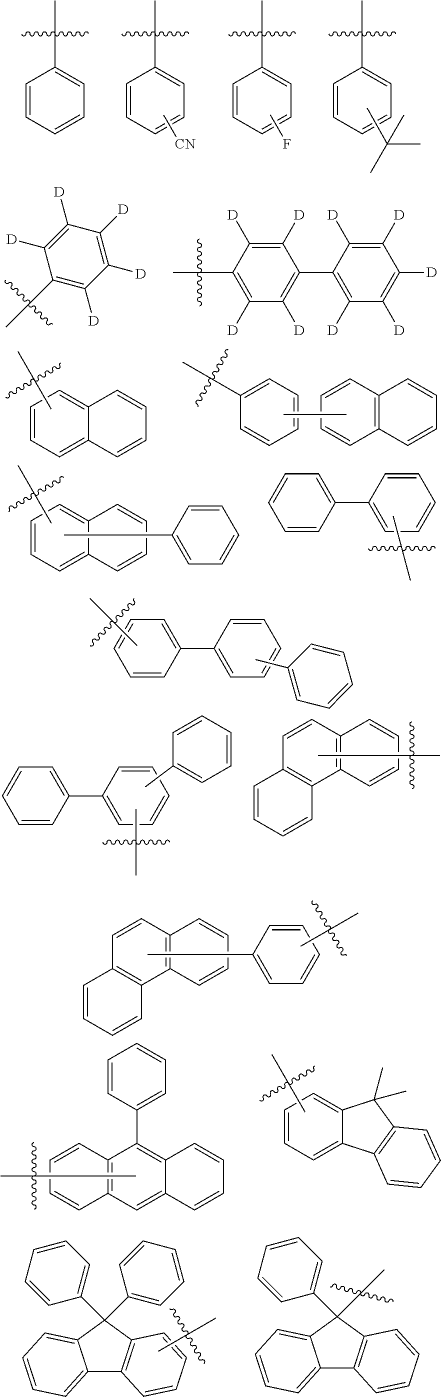

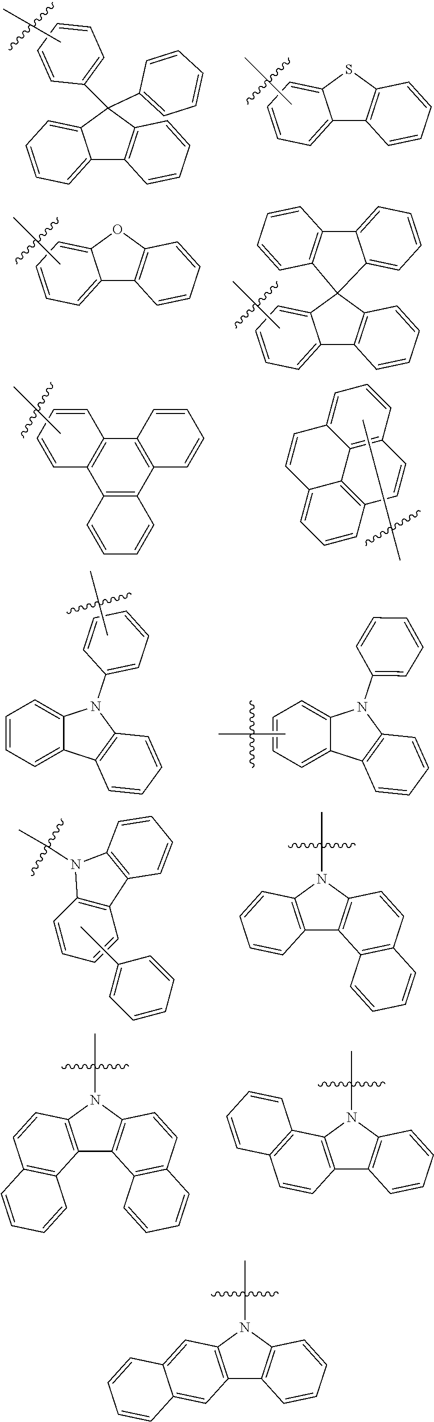

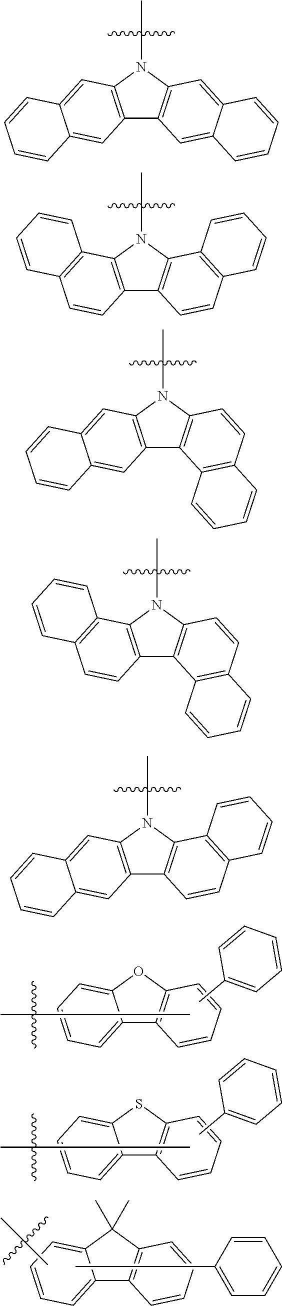

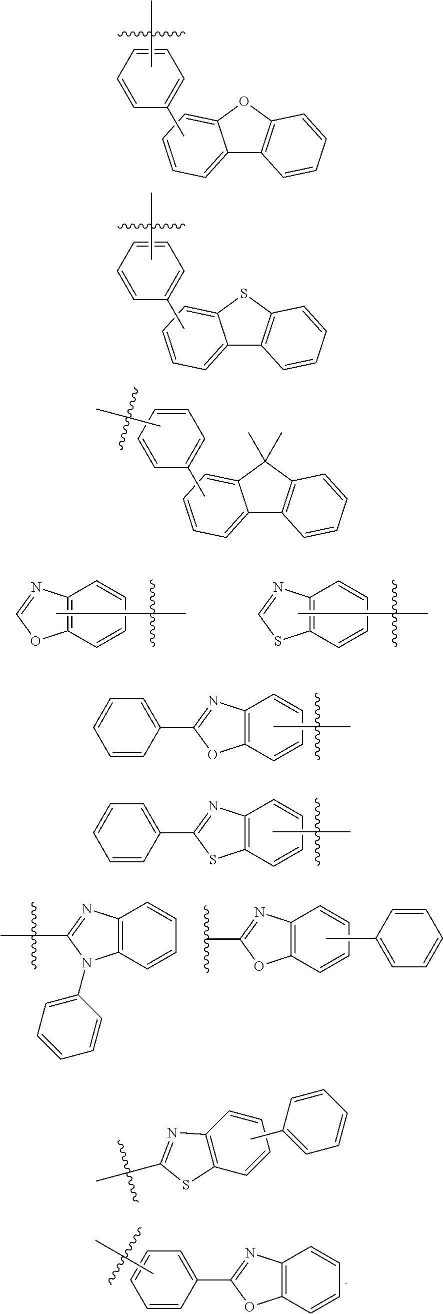

In some embodiments,

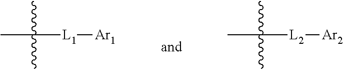

and are each independently selected from the following groups:

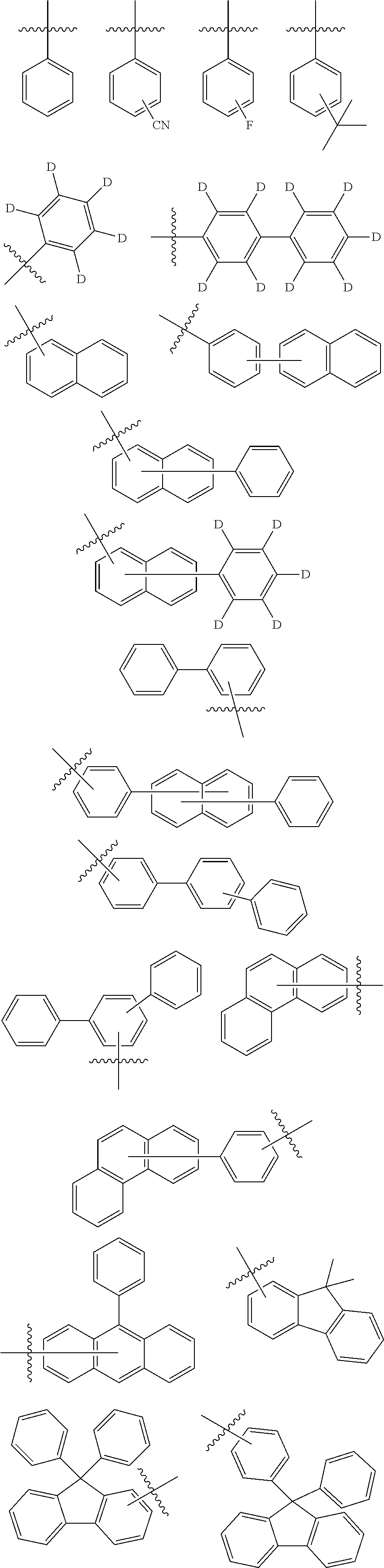

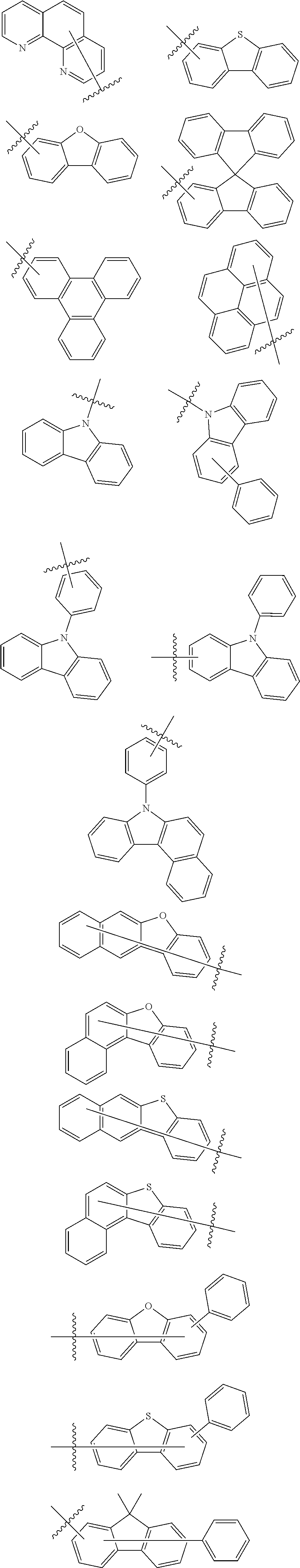

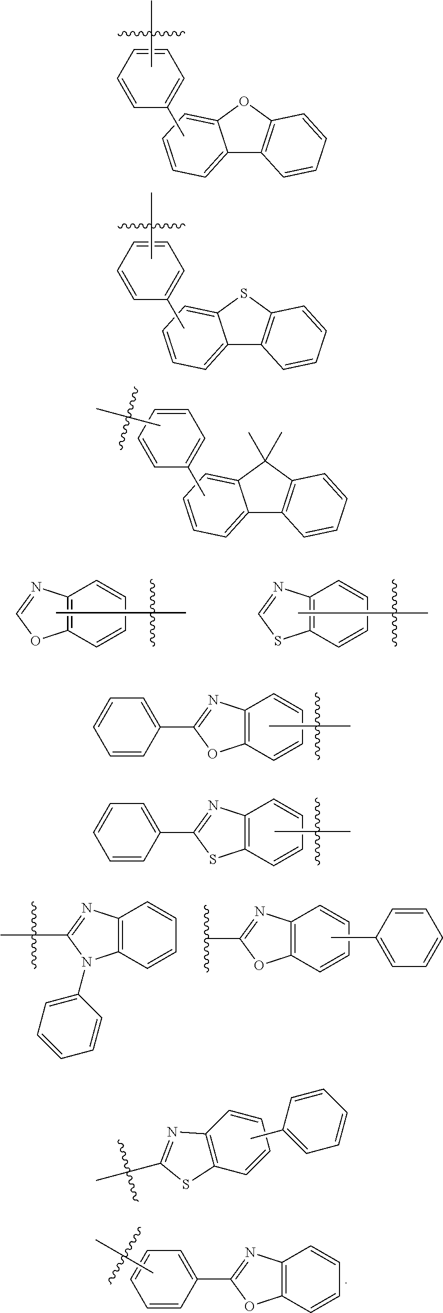

In some embodiments, group A is selected from the group consisting of the following groups:

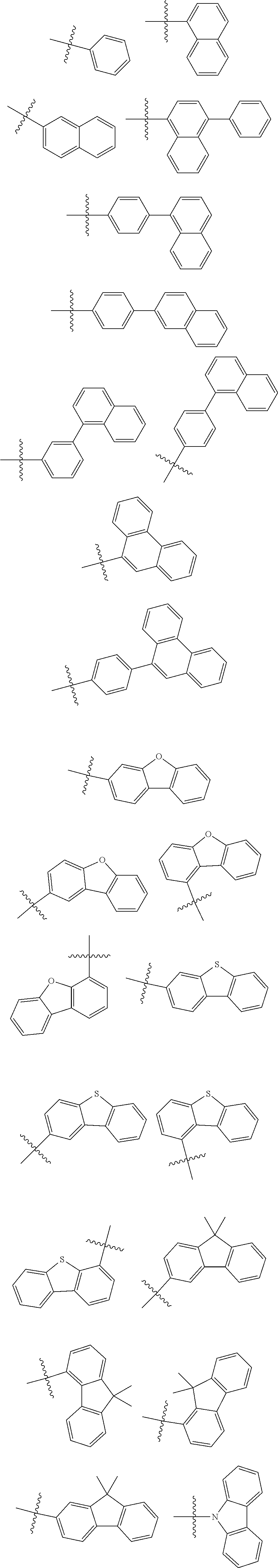

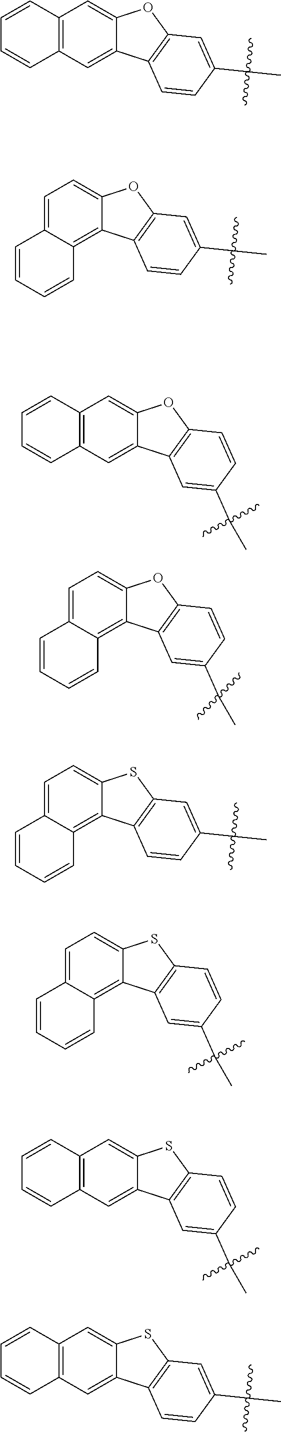

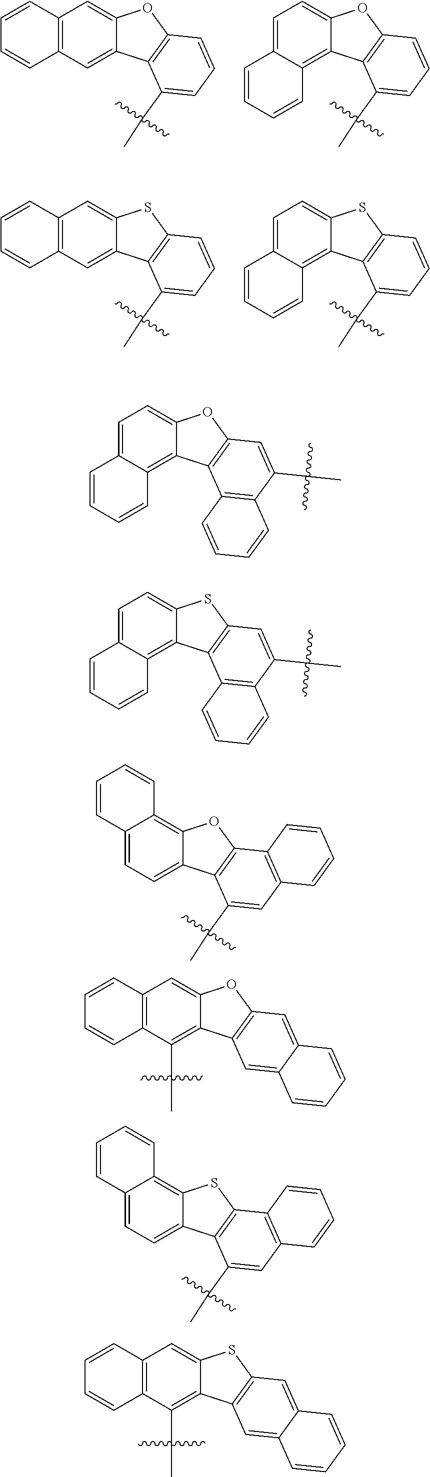

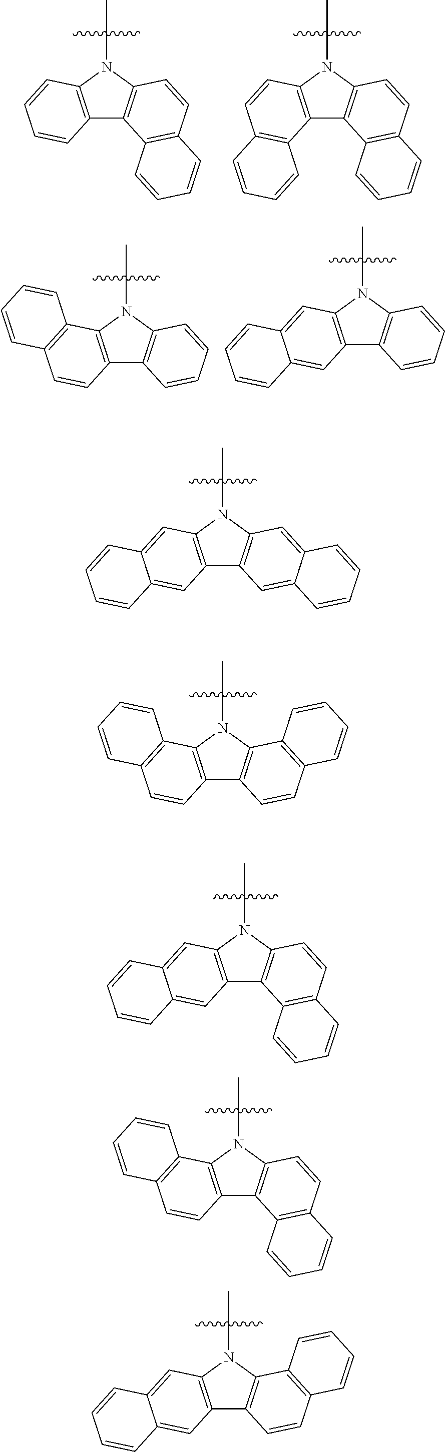

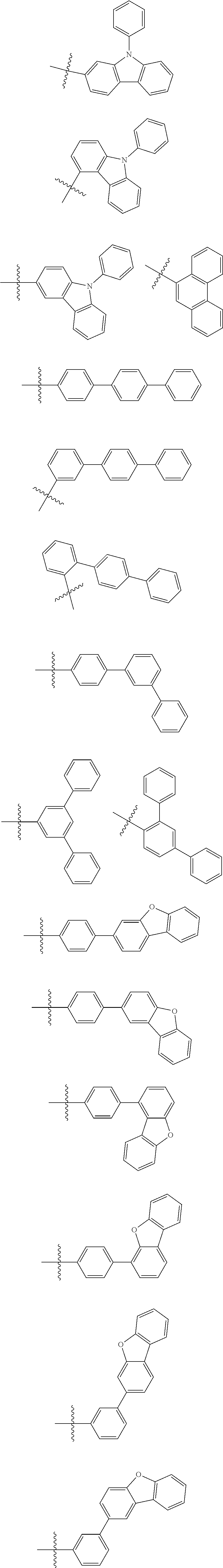

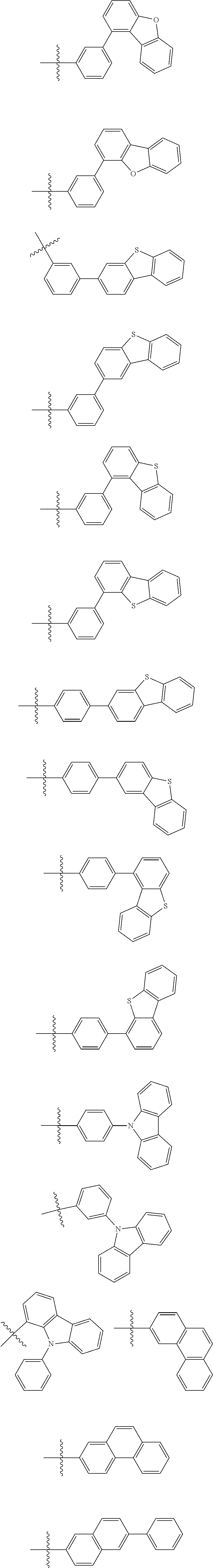

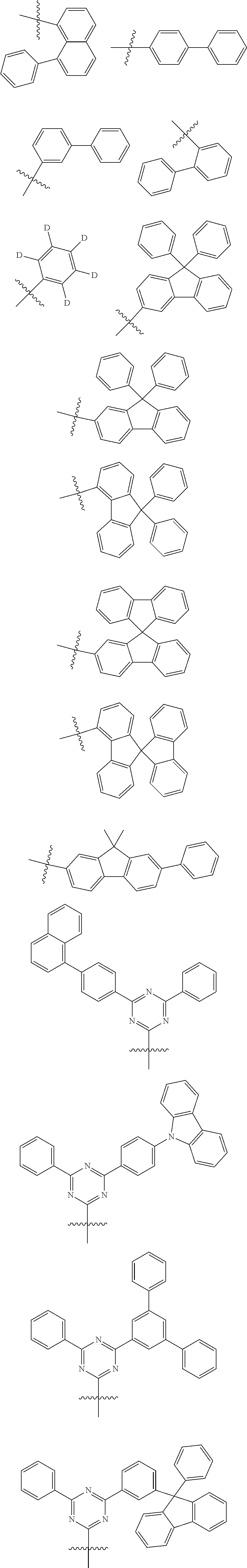

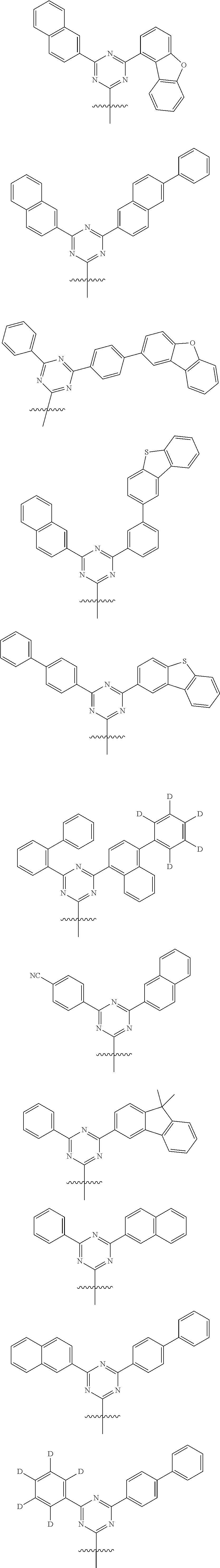

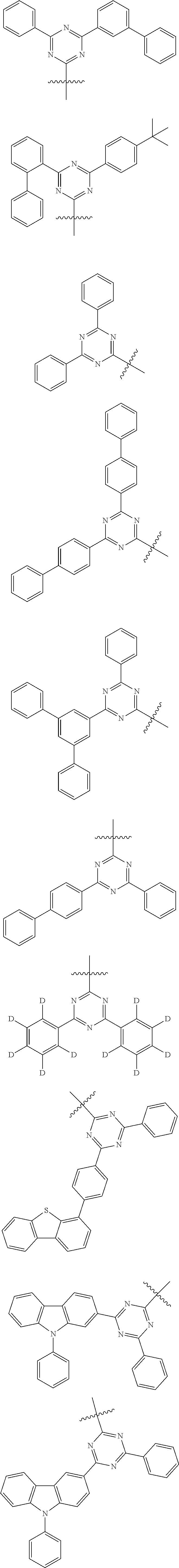

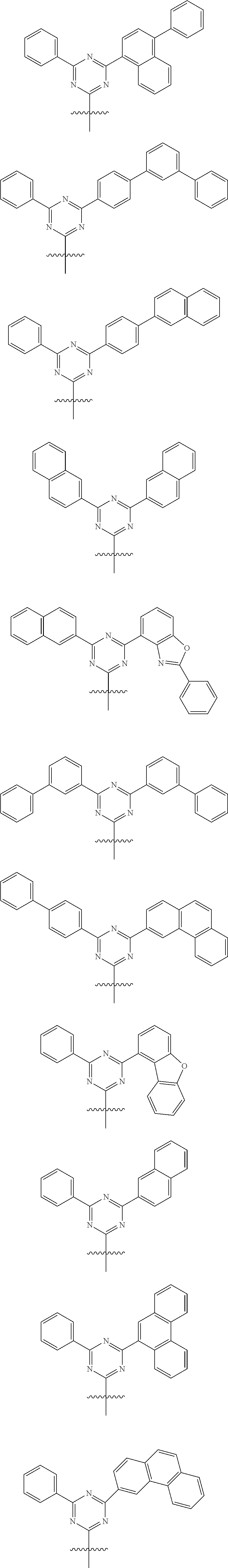

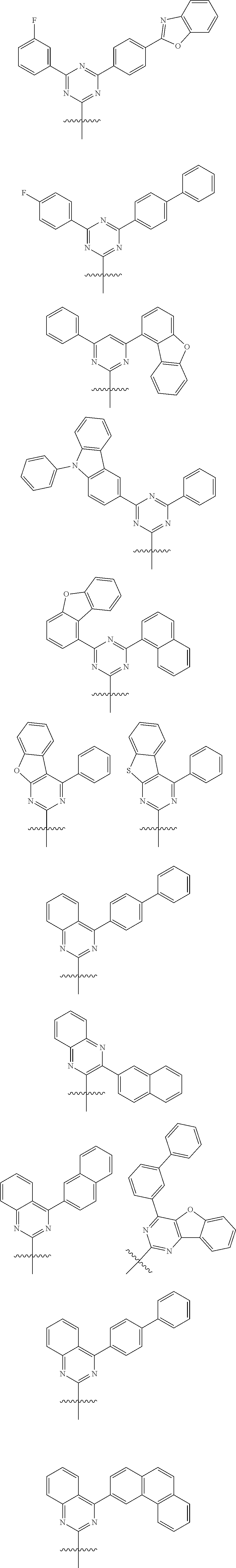

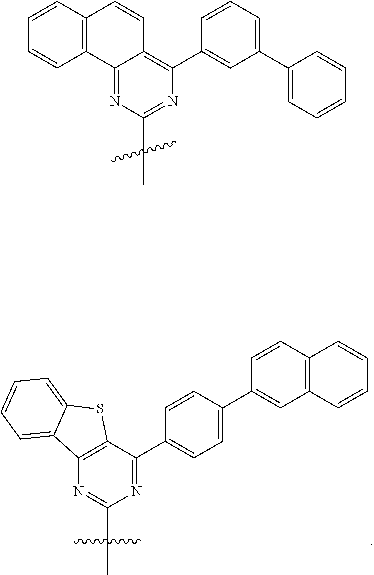 [SMILES: *c1nc(-c2cccc(-c3ccccc3)c2)c2ccc3ccccc3c2n1 ; *c1nc(-c2ccc(-c3ccc4ccccc4c3)cc2)c2sc3ccccc3c2n1]

Optionally, the nitrogen-containing compound is selected from the group consisting of the following compounds:

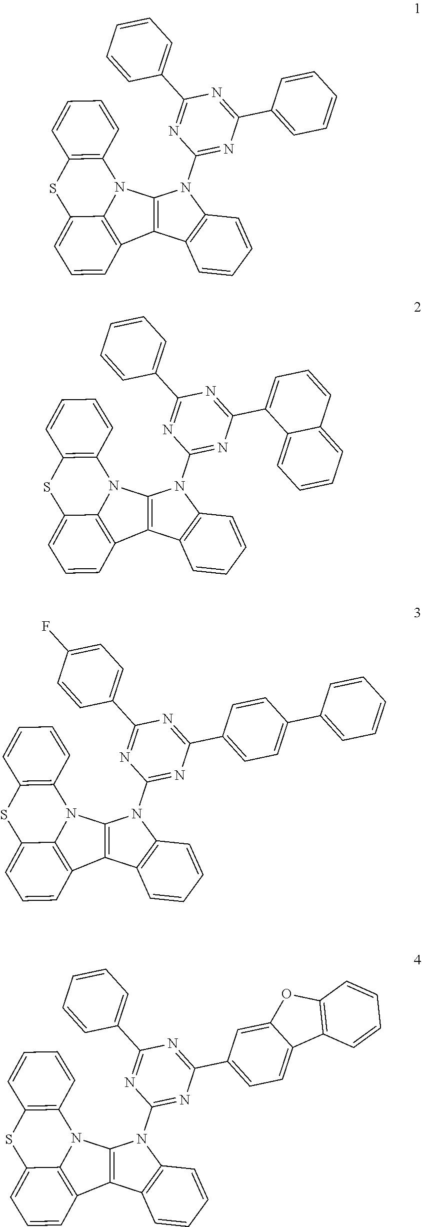 [SMILES: c1ccc(-c2nc(-c3ccccc3)nc(-n3c4ccccc4c4c5cccc6c5n(c43)-c3ccccc3S6)n2)cc1 ; c1ccc(-c2nc(-c3cccc4ccccc34)nc(-n3c4ccccc4c4c5cccc6c5n(c43)-c3ccccc3S6)n2)cc1 ; Fc1ccc(-c2nc(-c3ccc(-c4ccccc4)cc3)nc(-n3c4ccccc4c4c5cccc6c5n(c43)-c3ccccc3S6)n2)cc1 ; c1ccc(-c2nc(-c3ccc4c(c3)oc3ccccc34)nc(-n3c4ccccc4c4c5cccc6c5n(c43)-c3ccccc3S6)n2)cc1]

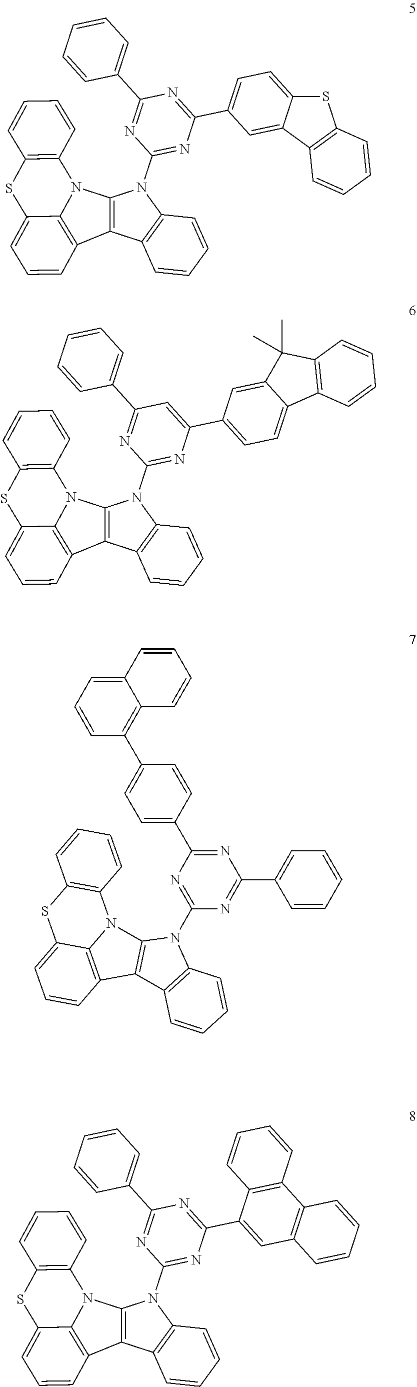 [SMILES: c1ccc(-c2nc(-c3ccc4sc5ccccc5c4c3)nc(-n3c4ccccc4c4c5cccc6c5n(c43)-c3ccccc3S6)n2)cc1 ; CC1(C)c2ccccc2-c2ccc(-c3cc(-c4ccccc4)nc(-n4c5ccccc5c5c6cccc7c6n(c54)-c4ccccc4S7)n3)cc21 ; c1ccc(-c2nc(-c3ccc(-c4cccc5ccccc45)cc3)nc(-n3c4ccccc4c4c5cccc6c5n(c43)-c3ccccc3S6)n2)cc1 ; c1ccc(-c2nc(-c3cc4ccccc4c4ccccc34)nc(-n3c4ccccc4c4c5cccc6c5n(c43)-c3ccccc3S6)n2)cc1]

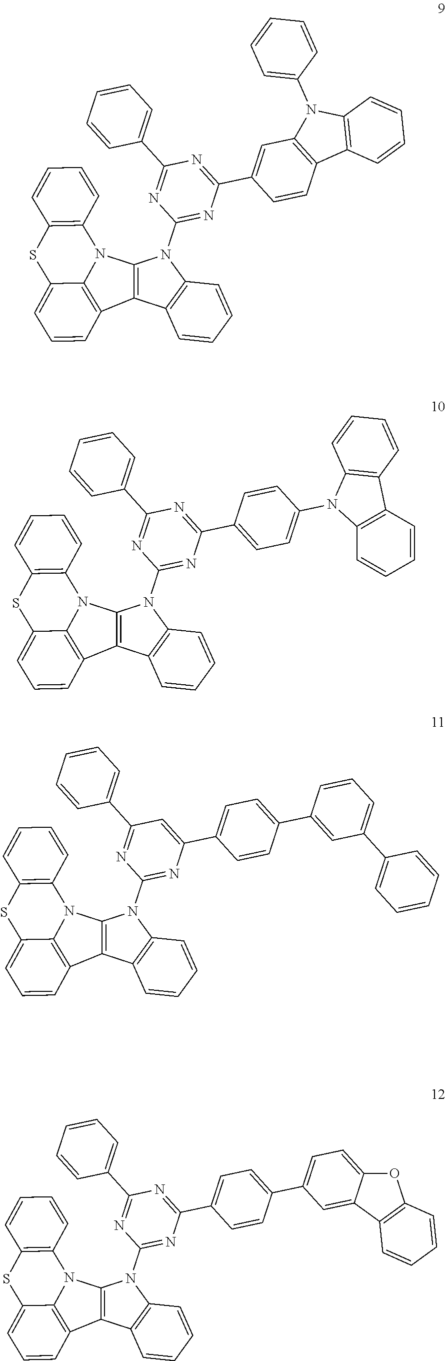 [SMILES: c1ccc(-c2nc(-c3ccc4c5ccccc5n(-c5ccccc5)c4c3)nc(-n3c4ccccc4c4c5cccc6c5n(c43)-c3ccccc3S6)n2)cc1 ; c1ccc(-c2nc(-c3ccc(-n4c5ccccc5c5ccccc54)cc3)nc(-n3c4ccccc4c4c5cccc6c5n(c43)-c3ccccc3S6)n2)cc1 ; c1ccc(-c2cccc(-c3ccc(-c4cc(-c5ccccc5)nc(-n5c6ccccc6c6c7cccc8c7n(c65)-c5ccccc5S8)n4)cc3)c2)cc1 ; c1ccc(-c2nc(-c3ccc(-c4ccc5oc6ccccc6c5c4)cc3)nc(-n3c4ccccc4c4c5cccc6c5n(c43)-c3ccccc3S6)n2)cc1]

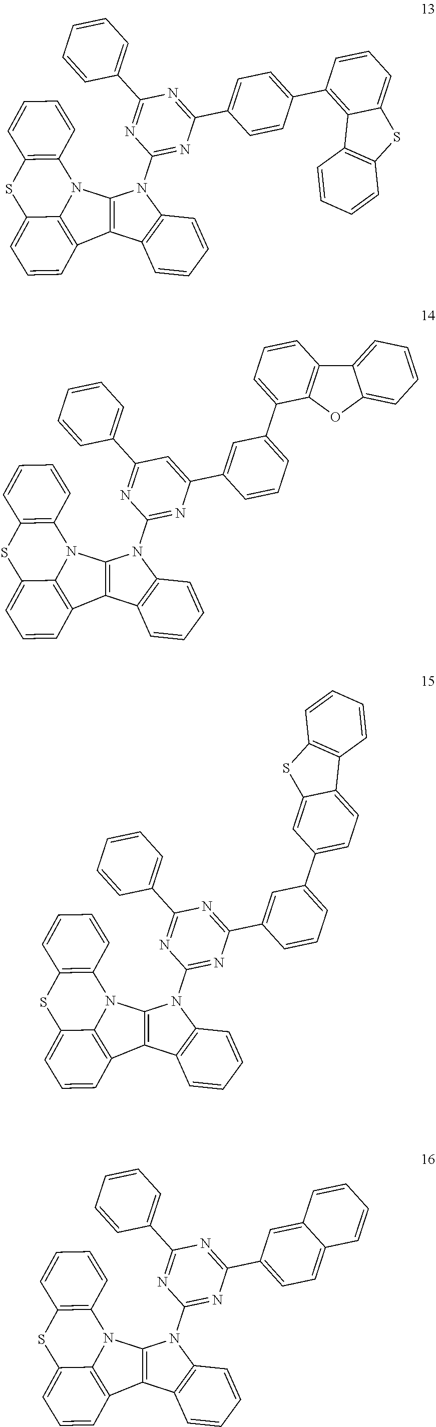 [SMILES: c1ccc(-c2nc(-c3ccc(-c4cccc5sc6ccccc6c45)cc3)nc(-n3c4ccccc4c4c5cccc6c5n(c43)-c3ccccc3S6)n2)cc1 ; c1ccc(-c2cc(-c3cccc(-c4cccc5c4oc4ccccc45)c3)nc(-n3c4ccccc4c4c5cccc6c5n(c43)-c3ccccc3S6)n2)cc1 ; c1ccc(-c2nc(-c3cccc(-c4ccc5c(c4)sc4ccccc45)c3)nc(-n3c4ccccc4c4c5cccc6c5n(c43)-c3ccccc3S6)n2)cc1 ; c1ccc(-c2nc(-c3ccc4ccccc4c3)nc(-n3c4ccccc4c4c5cccc6c5n(c43)-c3ccccc3S6)n2)cc1]

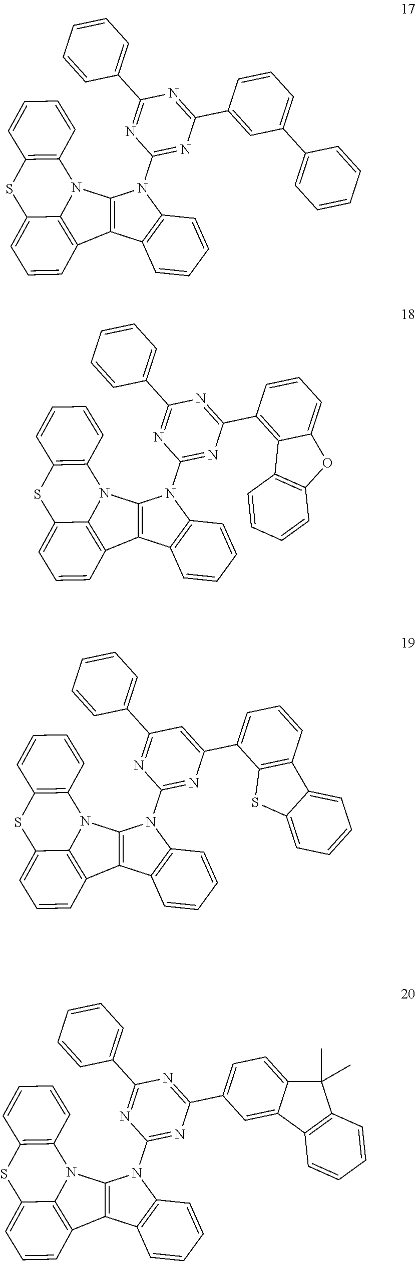 [SMILES: c1ccc(-c2cccc(-c3nc(-c4ccccc4)nc(-n4c5ccccc5c5c6cccc7c6n(c54)-c4ccccc4S7)n3)c2)cc1 ; c1ccc(-c2nc(-c3cccc4oc5ccccc5c34)nc(-n3c4ccccc4c4c5cccc6c5n(c43)-c3ccccc3S6)n2)cc1 ; c1ccc(-c2cc(-c3cccc4c3sc3ccccc34)nc(-n3c4ccccc4c4c5cccc6c5n(c43)-c3ccccc3S6)n2)cc1 ; CC1(C)c2ccccc2-c2cc(-c3nc(-c4ccccc4)nc(-n4c5ccccc5c5c6cccc7c6n(c54)-c4ccccc4S7)n3)ccc21]

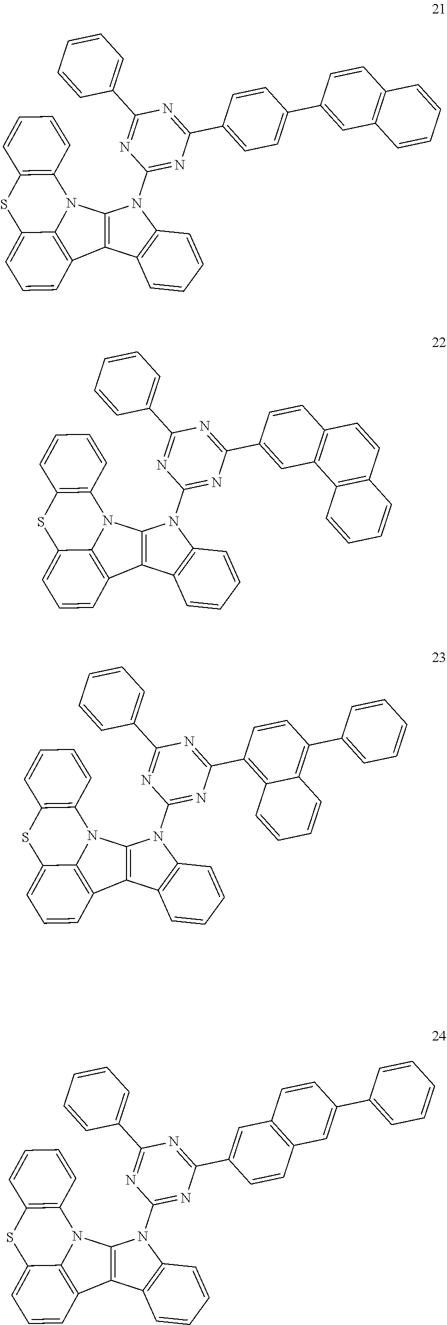 [SMILES: c1ccc(-c2nc(-c3ccc(-c4ccc5ccccc5c4)cc3)nc(-n3c4ccccc4c4c5cccc6c5n(c43)-c3ccccc3S6)n2)cc1 ; c1ccc(-c2nc(-c3ccc4ccc5ccccc5c4c3)nc(-n3c4ccccc4c4c5cccc6c5n(c43)-c3ccccc3S6)n2)cc1 ; c1ccc(-c2nc(-c3ccc(-c4ccccc4)c4ccccc34)nc(-n3c4ccccc4c4c5cccc6c5n(c43)-c3ccccc3S6)n2)cc1 ; c1ccc(-c2ccc3cc(-c4nc(-c5ccccc5)nc(-n5c6ccccc6c6c7cccc8c7n(c65)-c5ccccc5S8)n4)ccc3c2)cc1]

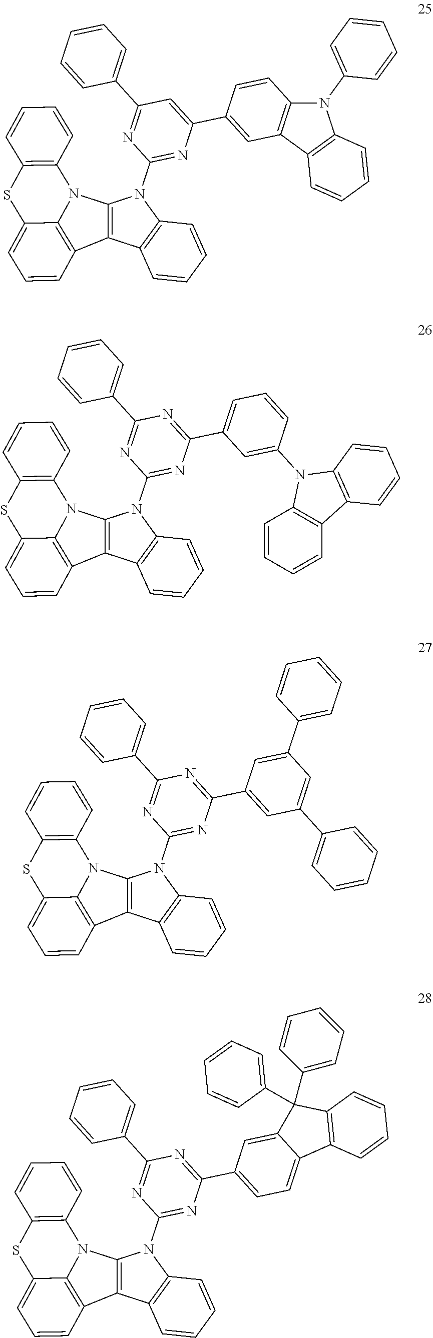 [SMILES: c1ccc(-c2cc(-c3ccc4c(c3)c3ccccc3n4-c3ccccc3)nc(-n3c4ccccc4c4c5cccc6c5n(c43)-c3ccccc3S6)n2)cc1 ; c1ccc(-c2nc(-c3cccc(-n4c5ccccc5c5ccccc54)c3)nc(-n3c4ccccc4c4c5cccc6c5n(c43)-c3ccccc3S6)n2)cc1 ; c1ccc(-c2cc(-c3ccccc3)cc(-c3nc(-c4ccccc4)nc(-n4c5ccccc5c5c6cccc7c6n(c54)-c4ccccc4S7)n3)c2)cc1 ; c1ccc(-c2nc(-c3ccc4c(c3)C(c3ccccc3)(c3ccccc3)c3ccccc3-4)nc(-n3c4ccccc4c4c5cccc6c5n(c43)-c3ccccc3S6)n2)cc1]

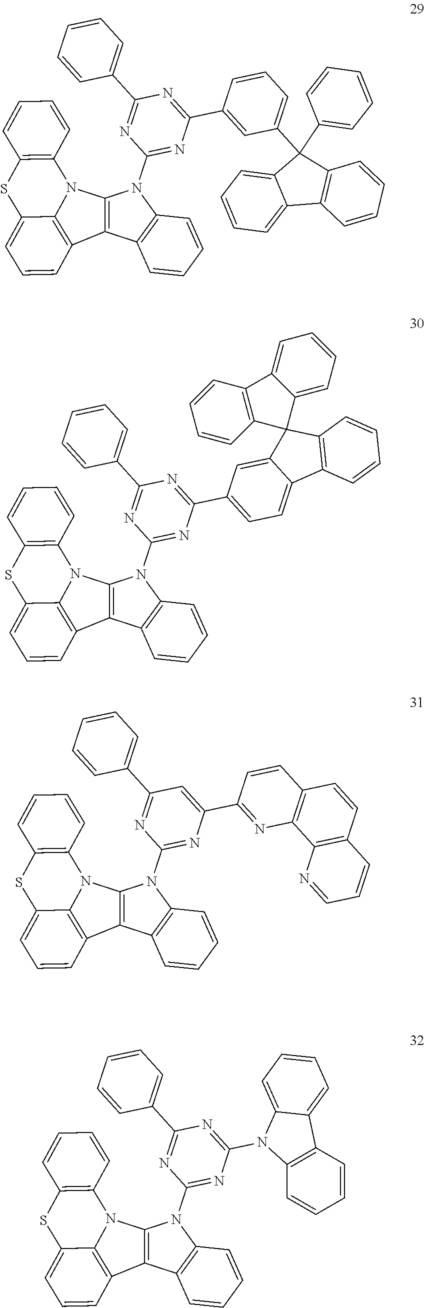 [SMILES: c1ccc(-c2nc(-c3cccc(C4(c5ccccc5)c5ccccc5-c5ccccc54)c3)nc(-n3c4ccccc4c4c5cccc6c5n(c43)-c3ccccc3S6)n2)cc1 ; c1ccc(-c2nc(-c3ccc4c(c3)C3(c5ccccc5-c5ccccc53)c3ccccc3-4)nc(-n3c4ccccc4c4c5cccc6c5n(c43)-c3ccccc3S6)n2)cc1 ; c1ccc(-c2cc(-c3ccc4ccc5cccnc5c4n3)nc(-n3c4ccccc4c4c5cccc6c5n(c43)-c3ccccc3S6)n2)cc1 ; c1ccc(-c2nc(-n3c4ccccc4c4ccccc43)nc(-n3c4ccccc4c4c5cccc6c5n(c43)-c3ccccc3S6)n2)cc1]

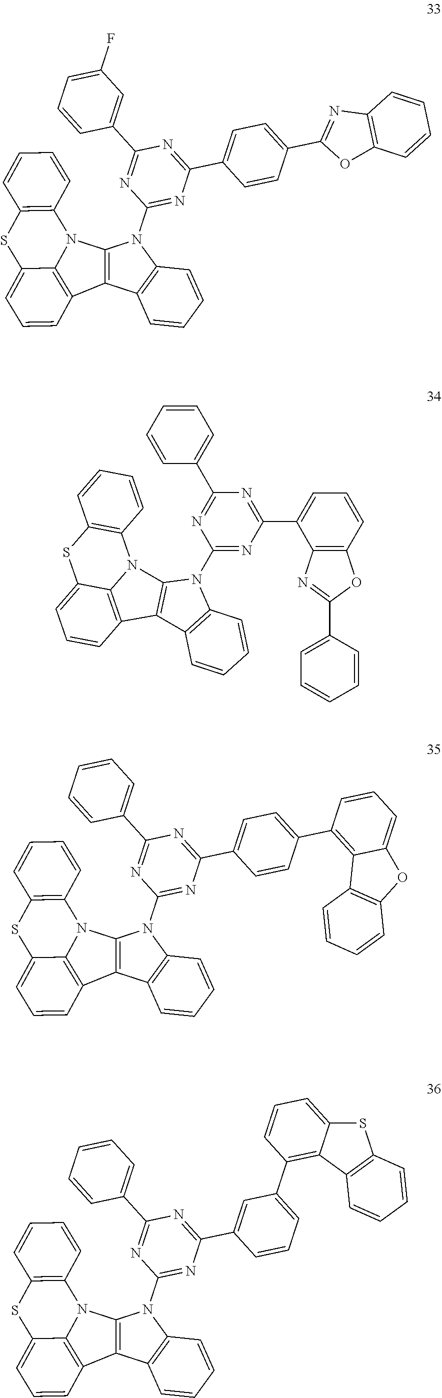 [SMILES: Fc1cccc(-c2nc(-c3ccc(-c4nc5ccccc5o4)cc3)nc(-n3c4ccccc4c4c5cccc6c5n(c43)-c3ccccc3S6)n2)c1 ; c1ccc(-c2nc(-c3cccc4oc(-c5ccccc5)nc34)nc(-n3c4ccccc4c4c5cccc6c5n(c43)-c3ccccc3S6)n2)cc1 ; c1ccc(-c2nc(-c3ccc(-c4cccc5oc6ccccc6c45)cc3)nc(-n3c4ccccc4c4c5cccc6c5n(c43)-c3ccccc3S6)n2)cc1 ; c1ccc(-c2nc(-c3cccc(-c4cccc5sc6ccccc6c45)c3)nc(-n3c4ccccc4c4c5cccc6c5n(c43)-c3ccccc3S6)n2)cc1]

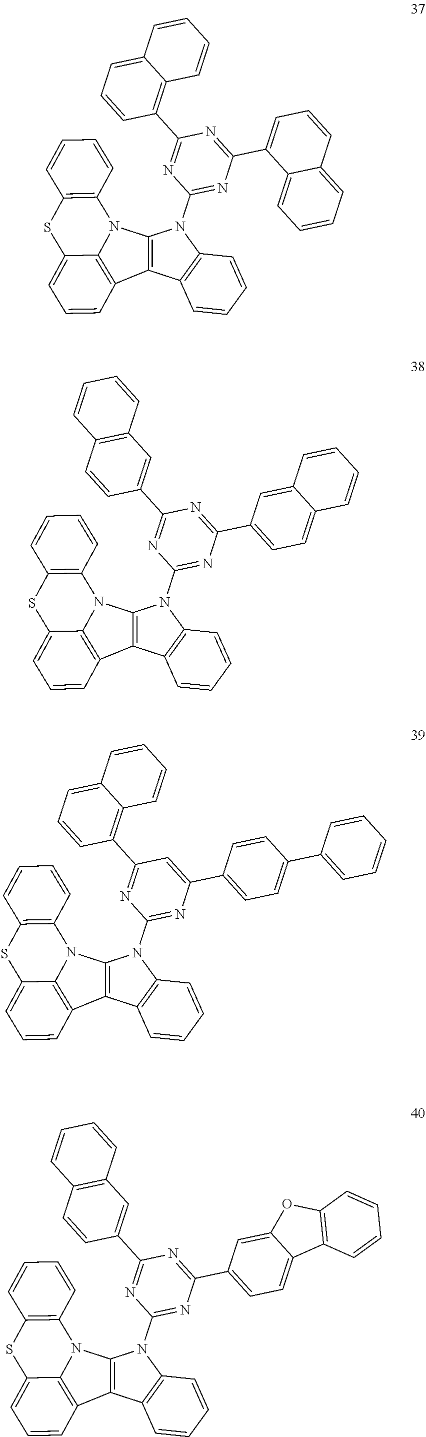 [SMILES: c1ccc2c(c1)Sc1cccc3c4c5ccccc5n(-c5nc(-c6cccc7ccccc67)nc(-c6cccc7ccccc67)n5)c4n-2c13 ; c1ccc2c(c1)Sc1cccc3c4c5ccccc5n(-c5nc(-c6ccc7ccccc7c6)nc(-c6ccc7ccccc7c6)n5)c4n-2c13 ; c1ccc(-c2ccc(-c3cc(-c4cccc5ccccc45)nc(-n4c5ccccc5c5c6cccc7c6n(c54)-c4ccccc4S7)n3)cc2)cc1 ; c1ccc2c(c1)Sc1cccc3c4c5ccccc5n(-c5nc(-c6ccc7ccccc7c6)nc(-c6ccc7c(c6)oc6ccccc67)n5)c4n-2c13]

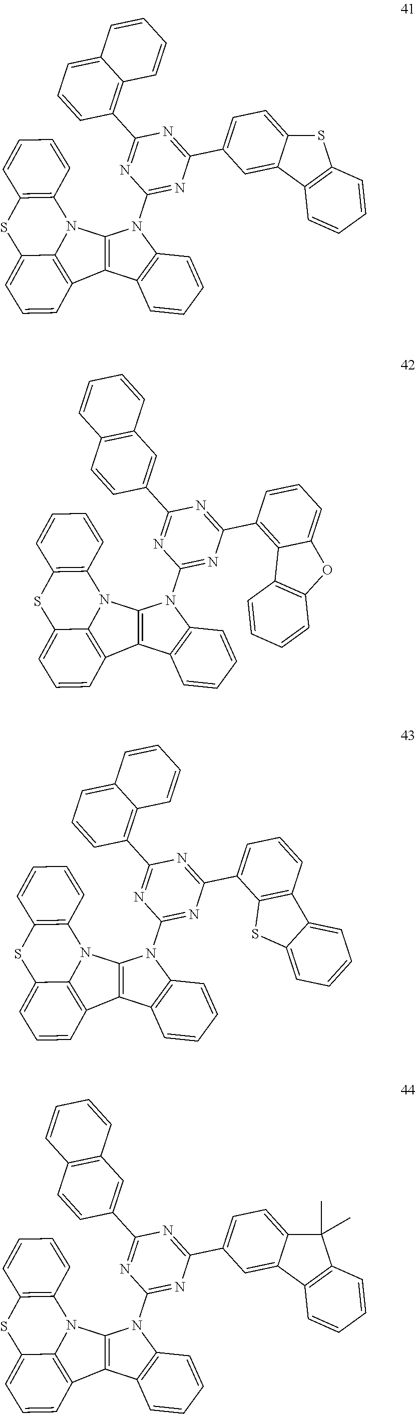 [SMILES: c1ccc2c(c1)Sc1cccc3c4c5ccccc5n(-c5nc(-c6ccc7sc8ccccc8c7c6)nc(-c6cccc7ccccc67)n5)c4n-2c13 ; c1ccc2c(c1)Sc1cccc3c4c5ccccc5n(-c5nc(-c6ccc7ccccc7c6)nc(-c6cccc7oc8ccccc8c67)n5)c4n-2c13 ; c1ccc2c(c1)Sc1cccc3c4c5ccccc5n(-c5nc(-c6cccc7ccccc67)nc(-c6cccc7c6sc6ccccc67)n5)c4n-2c13 ; CC1(C)c2ccccc2-c2cc(-c3nc(-c4ccc5ccccc5c4)nc(-n4c5ccccc5c5c6cccc7c6n(c54)-c4ccccc4S7)n3)ccc21]

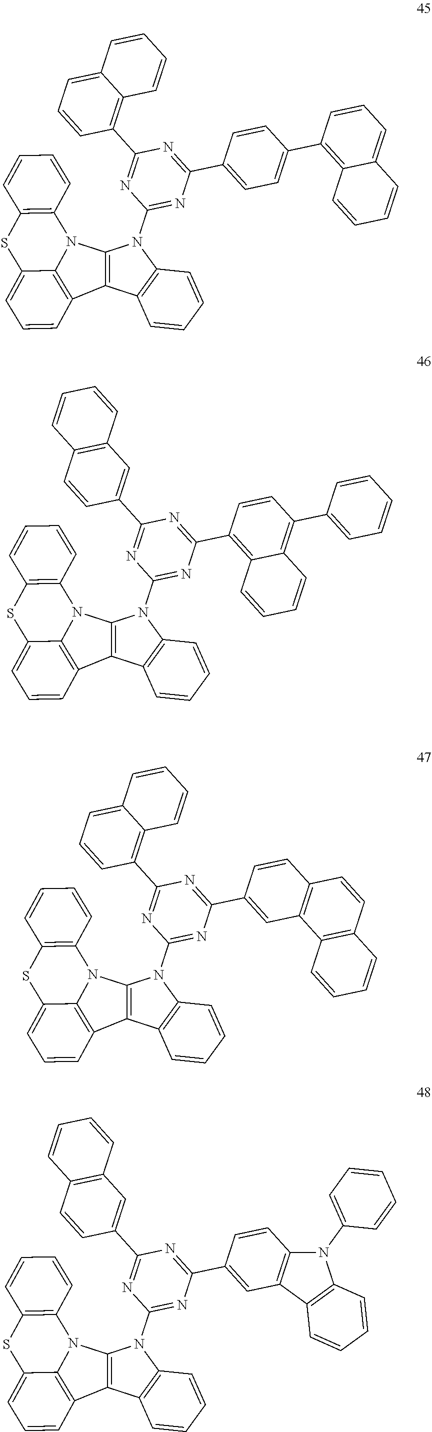 [SMILES: c1ccc2c(c1)Sc1cccc3c4c5ccccc5n(-c5nc(-c6ccc(-c7cccc8ccccc78)cc6)nc(-c6cccc7ccccc67)n5)c4n-2c13 ; c1ccc(-c2ccc(-c3nc(-c4ccc5ccccc5c4)nc(-n4c5ccccc5c5c6cccc7c6n(c54)-c4ccccc4S7)n3)c3ccccc23)cc1 ; c1ccc2c(c1)Sc1cccc3c4c5ccccc5n(-c5nc(-c6ccc7ccc8ccccc8c7c6)nc(-c6cccc7ccccc67)n5)c4n-2c13 ; c1ccc(-n2c3ccccc3c3cc(-c4nc(-c5ccc6ccccc6c5)nc(-n5c6ccccc6c6c7cccc8c7n(c65)-c5ccccc5S8)n4)ccc32)cc1]

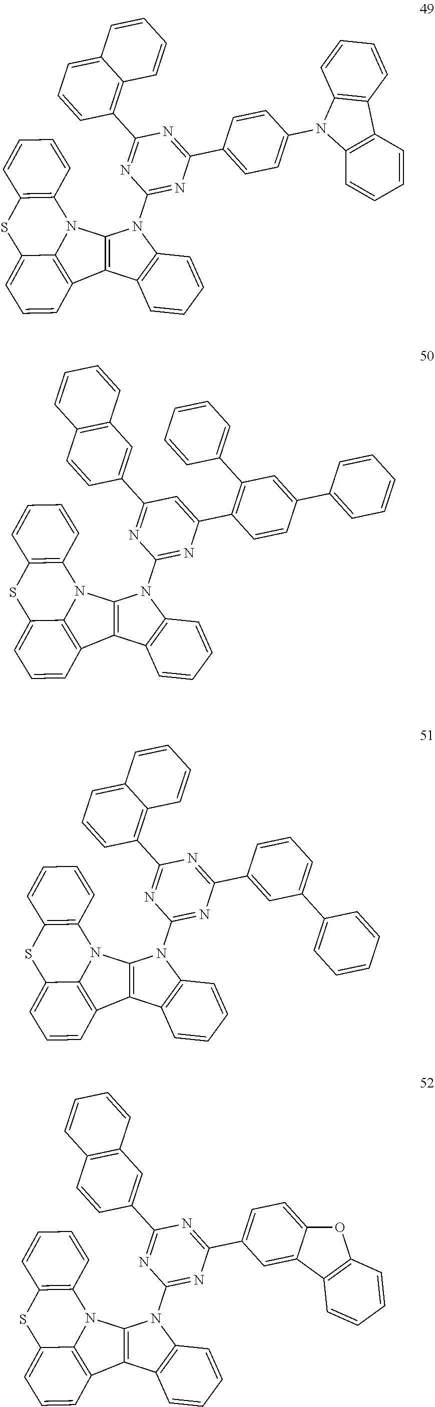 [SMILES: c1ccc2c(c1)Sc1cccc3c4c5ccccc5n(-c5nc(-c6ccc(-n7c8ccccc8c8ccccc87)cc6)nc(-c6cccc7ccccc67)n5)c4n-2c13 ; c1ccc(-c2ccc(-c3cc(-c4ccc5ccccc5c4)nc(-n4c5ccccc5c5c6cccc7c6n(c54)-c4ccccc4S7)n3)c(-c3ccccc3)c2)cc1 ; c1ccc(-c2cccc(-c3nc(-c4cccc5ccccc45)nc(-n4c5ccccc5c5c6cccc7c6n(c54)-c4ccccc4S7)n3)c2)cc1 ; c1ccc2c(c1)Sc1cccc3c4c5ccccc5n(-c5nc(-c6ccc7ccccc7c6)nc(-c6ccc7oc8ccccc8c7c6)n5)c4n-2c13]

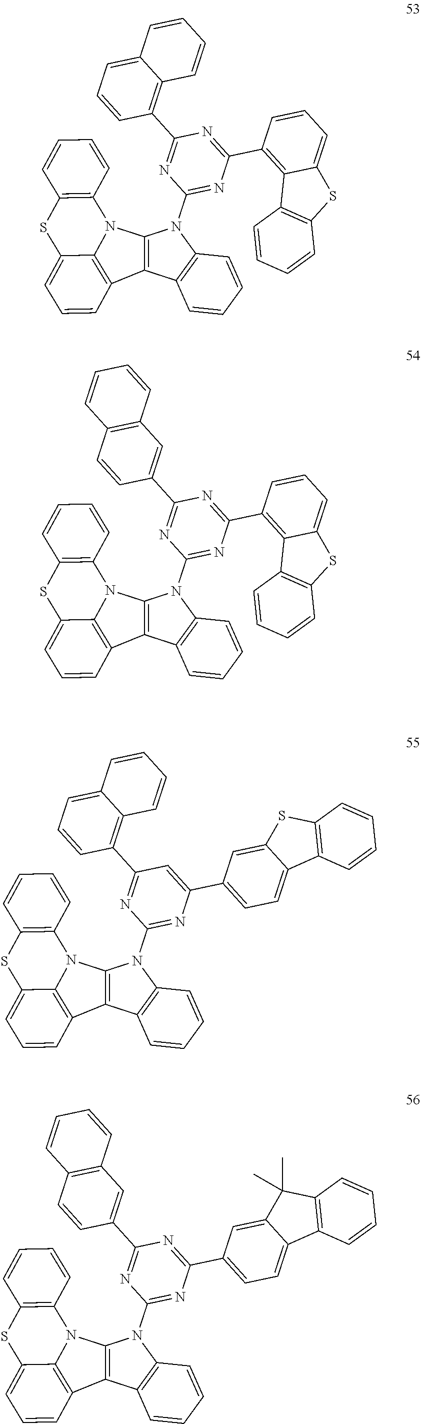 [SMILES: c1ccc2c(c1)Sc1cccc3c4c5ccccc5n(-c5nc(-c6cccc7ccccc67)nc(-c6cccc7sc8ccccc8c67)n5)c4n-2c13 ; c1ccc2c(c1)Sc1cccc3c4c5ccccc5n(-c5nc(-c6ccc7ccccc7c6)nc(-c6cccc7sc8ccccc8c67)n5)c4n-2c13 ; c1ccc2c(c1)Sc1cccc3c4c5ccccc5n(-c5nc(-c6ccc7c(c6)sc6ccccc67)cc(-c6cccc7ccccc67)n5)c4n-2c13 ; CC1(C)c2ccccc2-c2ccc(-c3nc(-c4ccc5ccccc5c4)nc(-n4c5ccccc5c5c6cccc7c6n(c54)-c4ccccc4S7)n3)cc21]

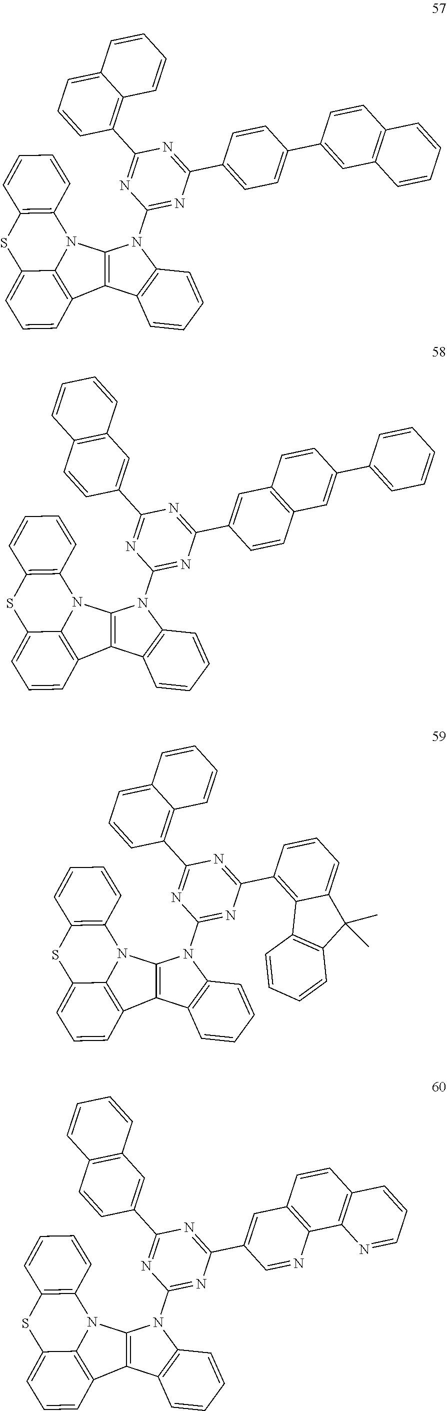 [SMILES: c1ccc2c(c1)Sc1cccc3c4c5ccccc5n(-c5nc(-c6ccc(-c7ccc8ccccc8c7)cc6)nc(-c6cccc7ccccc67)n5)c4n-2c13 ; c1ccc(-c2ccc3cc(-c4nc(-c5ccc6ccccc6c5)nc(-n5c6ccccc6c6c7cccc8c7n(c65)-c5ccccc5S8)n4)ccc3c2)cc1 ; CC1(C)c2ccccc2-c2c(-c3nc(-c4cccc5ccccc45)nc(-n4c5ccccc5c5c6cccc7c6n(c54)-c4ccccc4S7)n3)cccc21 ; c1ccc2c(c1)Sc1cccc3c4c5ccccc5n(-c5nc(-c6ccc7ccccc7c6)nc(-c6cnc7c(ccc8cccnc87)c6)n5)c4n-2c13]

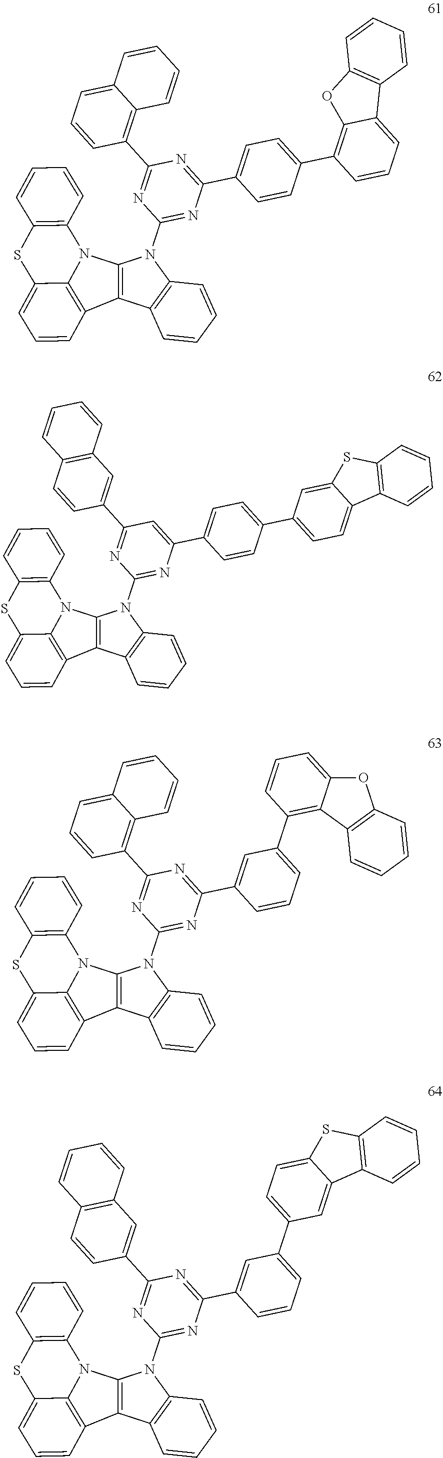 [SMILES: c1ccc2c(c1)Sc1cccc3c4c5ccccc5n(-c5nc(-c6ccc(-c7cccc8c7oc7ccccc78)cc6)nc(-c6cccc7ccccc67)n5)c4n-2c13 ; c1ccc2c(c1)Sc1cccc3c4c5ccccc5n(-c5nc(-c6ccc(-c7ccc8c(c7)sc7ccccc78)cc6)cc(-c6ccc7ccccc7c6)n5)c4n-2c13 ; c1cc(-c2nc(-c3cccc4ccccc34)nc(-n3c4ccccc4c4c5cccc6c5n(c43)-c3ccccc3S6)n2)cc(-c2cccc3oc4ccccc4c23)c1 ; c1cc(-c2ccc3sc4ccccc4c3c2)cc(-c2nc(-c3ccc4ccccc4c3)nc(-n3c4ccccc4c4c5cccc6c5n(c43)-c3ccccc3S6)n2)c1]

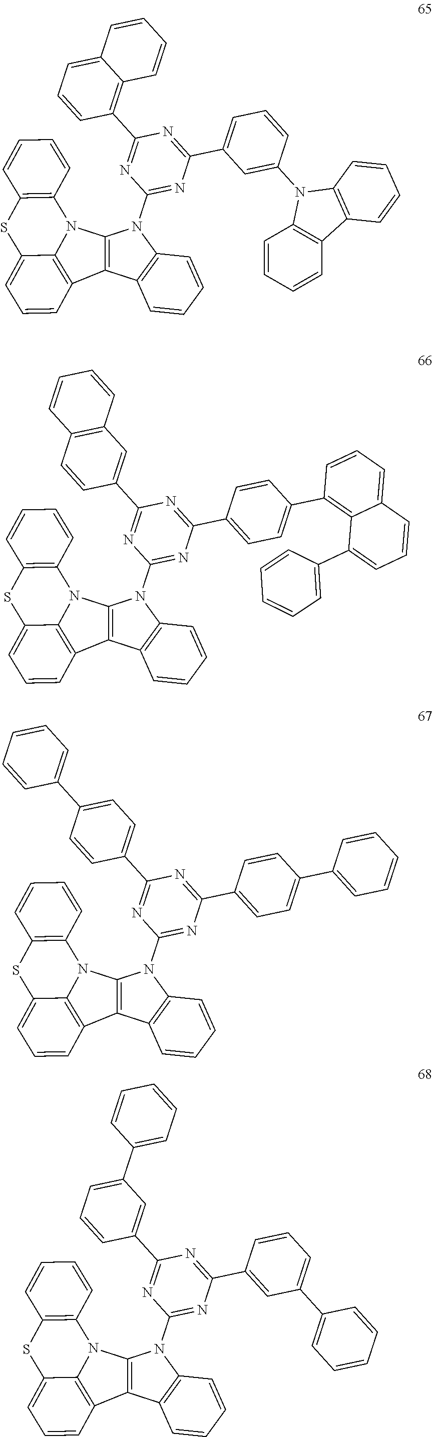

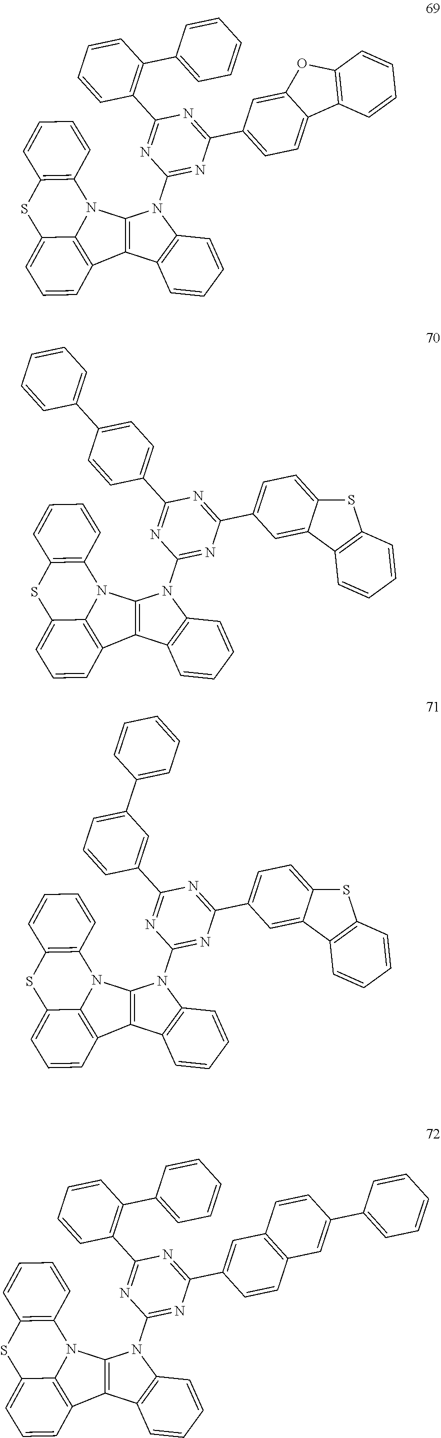 [SMILES: c1ccc(-c2ccccc2-c2nc(-c3ccc4c(c3)oc3ccccc34)nc(-n3c4ccccc4c4c5cccc6c5n(c43)-c3ccccc3S6)n2)cc1 ; c1ccc(-c2ccc(-c3nc(-c4ccc5sc6ccccc6c5c4)nc(-n4c5ccccc5c5c6cccc7c6n(c54)-c4ccccc4S7)n3)cc2)cc1 ; c1ccc(-c2cccc(-c3nc(-c4ccc5sc6ccccc6c5c4)nc(-n4c5ccccc5c5c6cccc7c6n(c54)-c4ccccc4S7)n3)c2)cc1 ; c1ccc(-c2ccc3cc(-c4nc(-c5ccccc5-c5ccccc5)nc(-n5c6ccccc6c6c7cccc8c7n(c65)-c5ccccc5S8)n4)ccc3c2)cc1]

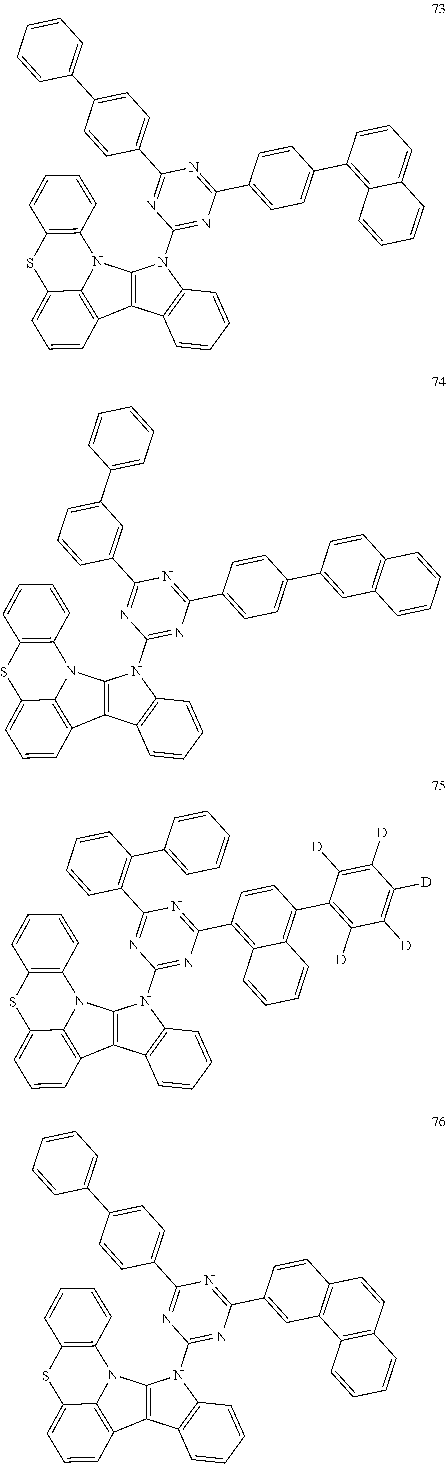

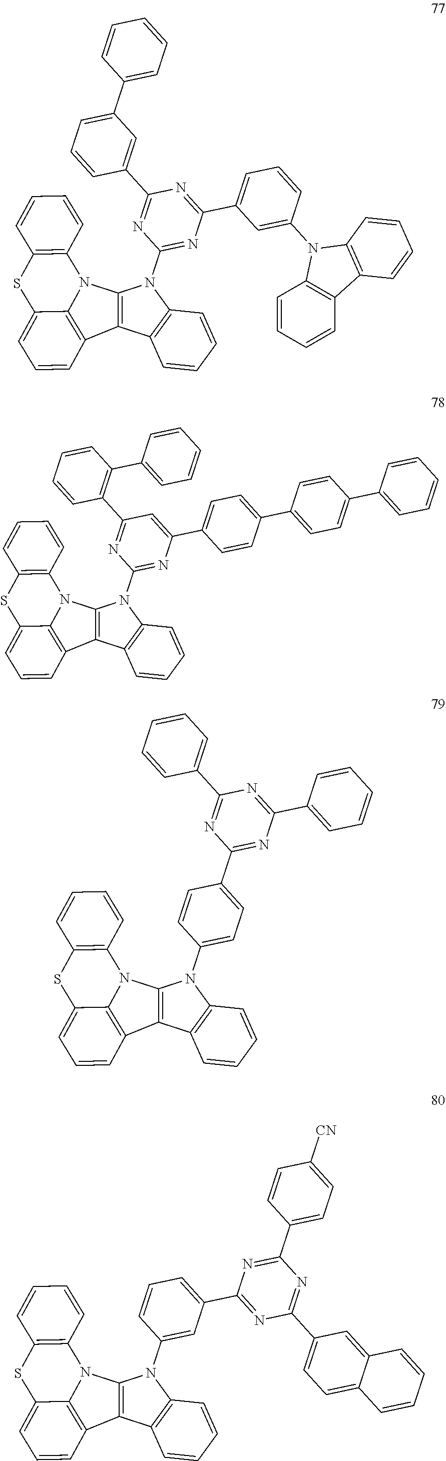

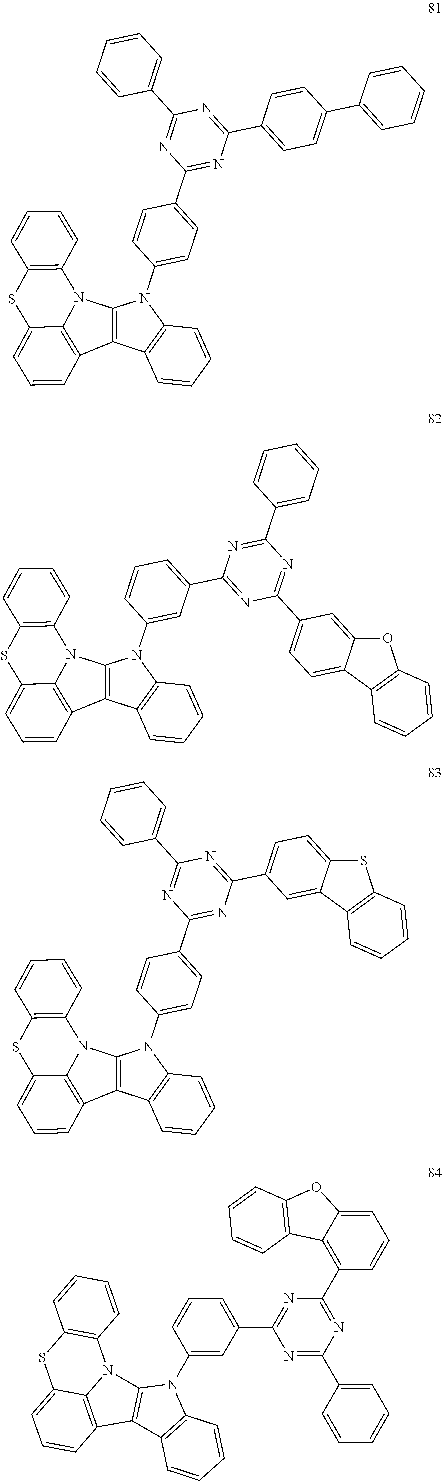

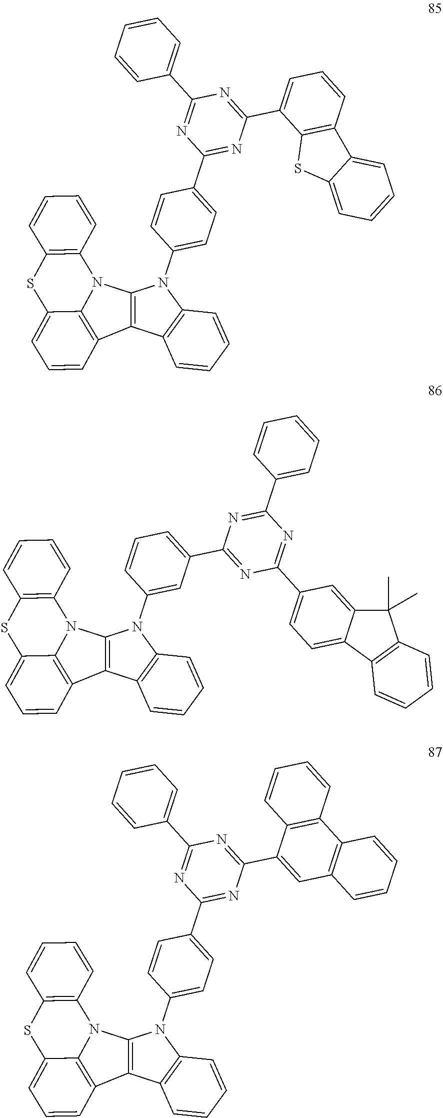 [SMILES: c1ccc(-c2nc(-c3ccc(-n4c5ccccc5c5c6cccc7c6n(c54)-c4ccccc4S7)cc3)nc(-c3cccc4c3sc3ccccc34)n2)cc1 ; CC1(C)c2ccccc2-c2ccc(-c3nc(-c4ccccc4)nc(-c4cccc(-n5c6ccccc6c6c7cccc8c7n(c65)-c5ccccc5S8)c4)n3)cc21 ; c1ccc(-c2nc(-c3ccc(-n4c5ccccc5c5c6cccc7c6n(c54)-c4ccccc4S7)cc3)nc(-c3cc4ccccc4c4ccccc34)n2)cc1]

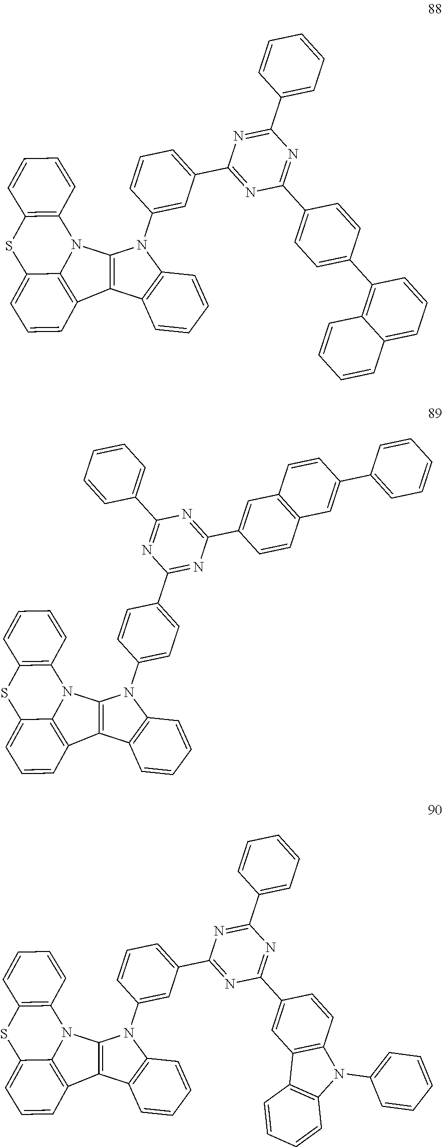 [SMILES: c1ccc(-c2nc(-c3ccc(-c4cccc5ccccc45)cc3)nc(-c3cccc(-n4c5ccccc5c5c6cccc7c6n(c54)-c4ccccc4S7)c3)n2)cc1 ; c1ccc(-c2ccc3cc(-c4nc(-c5ccccc5)nc(-c5ccc(-n6c7ccccc7c7c8cccc9c8n(c76)-c6ccccc6S9)cc5)n4)ccc3c2)cc1 ; c1ccc(-c2nc(-c3cccc(-n4c5ccccc5c5c6cccc7c6n(c54)-c4ccccc4S7)c3)nc(-c3ccc4c(c3)c3ccccc3n4-c3ccccc3)n2)cc1]

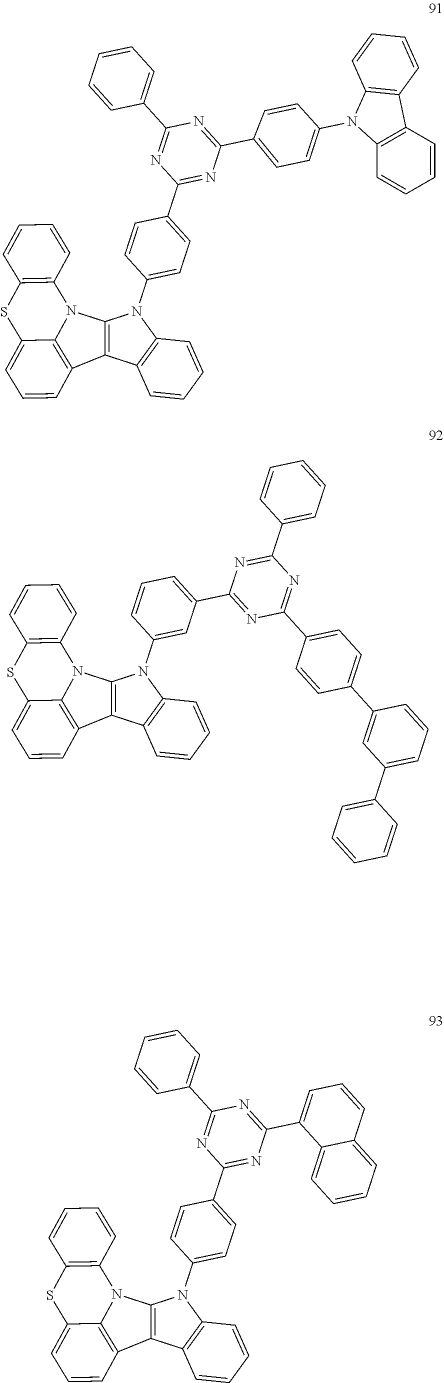 [SMILES: c1ccc(-c2nc(-c3ccc(-n4c5ccccc5c5ccccc54)cc3)nc(-c3ccc(-n4c5ccccc5c5c6cccc7c6n(c54)-c4ccccc4S7)cc3)n2)cc1 ; c1ccc(-c2cccc(-c3ccc(-c4nc(-c5ccccc5)nc(-c5cccc(-n6c7ccccc7c7c8cccc9c8n(c76)-c6ccccc6S9)c5)n4)cc3)c2)cc1 ; c1ccc(-c2nc(-c3ccc(-n4c5ccccc5c5c6cccc7c6n(c54)-c4ccccc4S7)cc3)nc(-c3cccc4ccccc34)n2)cc1]

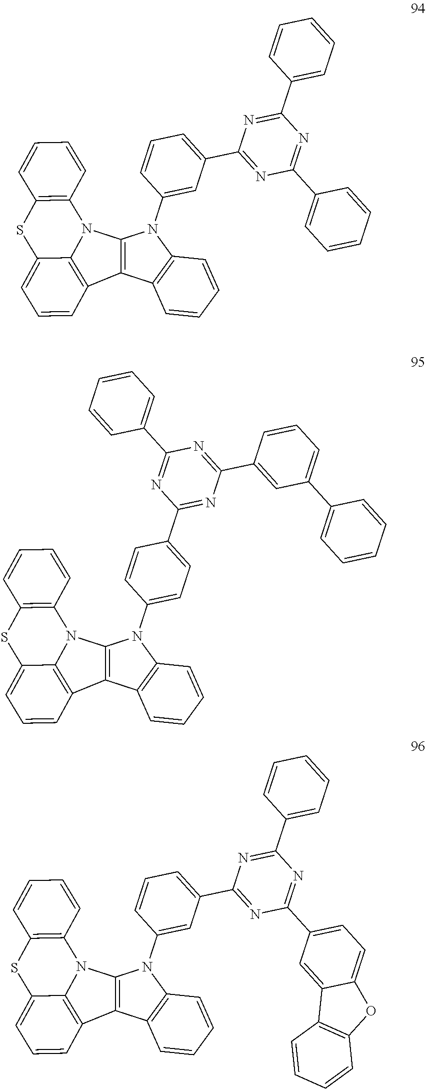 [SMILES: c1ccc(-c2nc(-c3ccccc3)nc(-c3cccc(-n4c5ccccc5c5c6cccc7c6n(c54)-c4ccccc4S7)c3)n2)cc1 ; c1ccc(-c2cccc(-c3nc(-c4ccccc4)nc(-c4ccc(-n5c6ccccc6c6c7cccc8c7n(c65)-c5ccccc5S8)cc4)n3)c2)cc1 ; c1ccc(-c2nc(-c3cccc(-n4c5ccccc5c5c6cccc7c6n(c54)-c4ccccc4S7)c3)nc(-c3ccc4oc5ccccc5c4c3)n2)cc1]

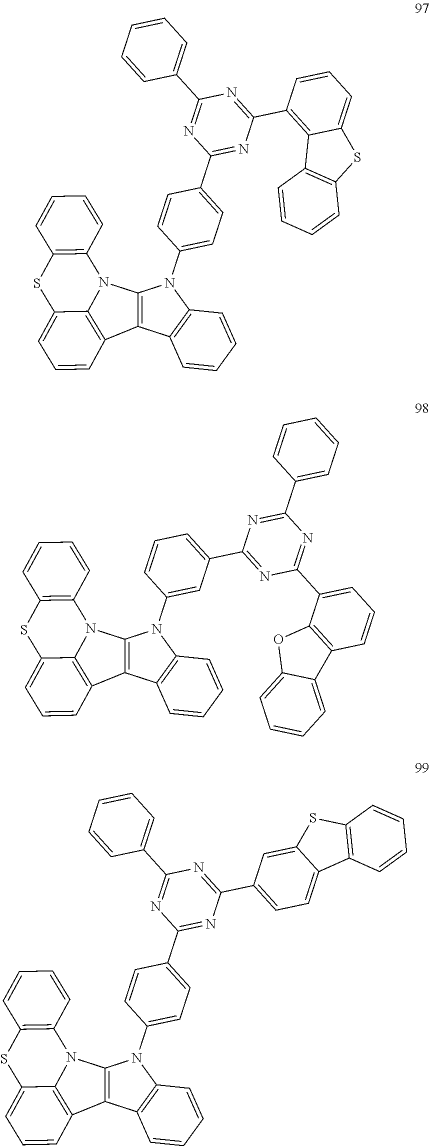 [SMILES: c1ccc(-c2nc(-c3ccc(-n4c5ccccc5c5c6cccc7c6n(c54)-c4ccccc4S7)cc3)nc(-c3cccc4sc5ccccc5c34)n2)cc1 ; c1ccc(-c2nc(-c3cccc(-n4c5ccccc5c5c6cccc7c6n(c54)-c4ccccc4S7)c3)nc(-c3cccc4c3oc3ccccc34)n2)cc1 ; c1ccc(-c2nc(-c3ccc(-n4c5ccccc5c5c6cccc7c6n(c54)-c4ccccc4S7)cc3)nc(-c3ccc4c(c3)sc3ccccc34)n2)cc1]

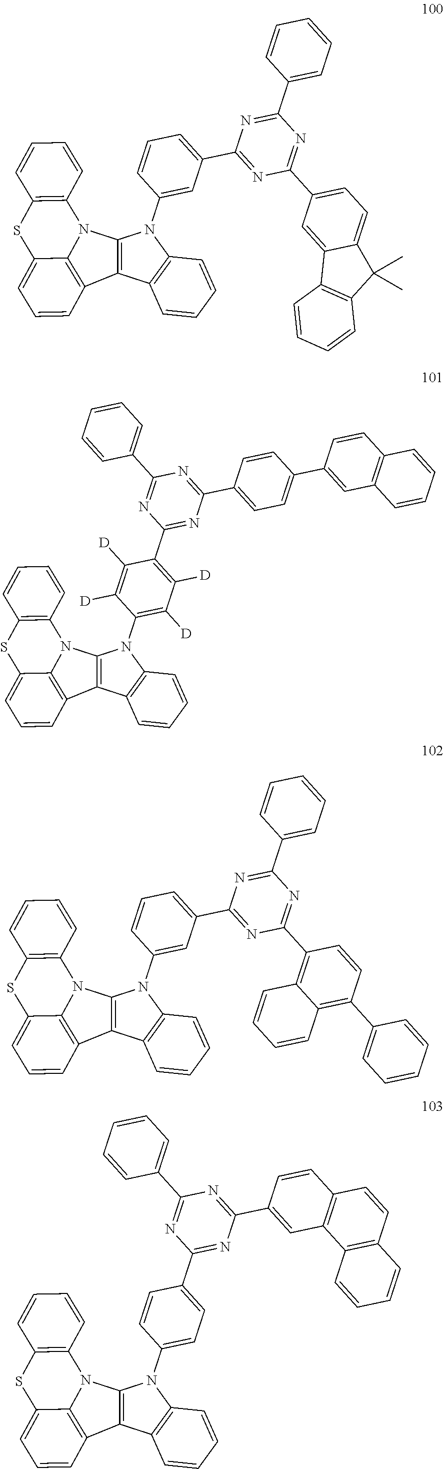

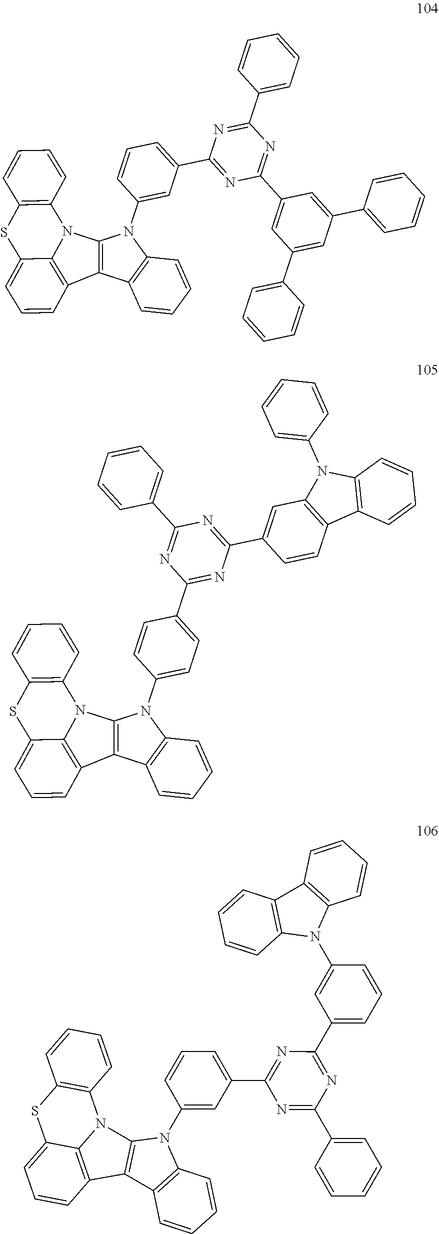

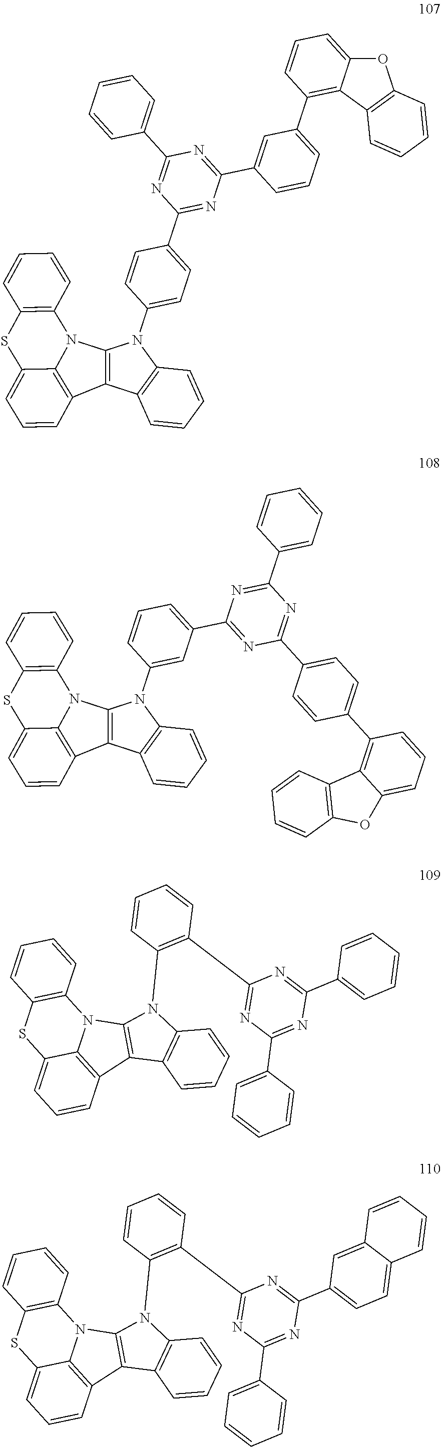 [SMILES: c1ccc(-c2nc(-c3ccc(-n4c5ccccc5c5c6cccc7c6n(c54)-c4ccccc4S7)cc3)nc(-c3cccc(-c4cccc5oc6ccccc6c45)c3)n2)cc1 ; c1ccc(-c2nc(-c3ccc(-c4cccc5oc6ccccc6c45)cc3)nc(-c3cccc(-n4c5ccccc5c5c6cccc7c6n(c54)-c4ccccc4S7)c3)n2)cc1 ; c1ccc(-c2nc(-c3ccccc3)nc(-c3ccccc3-n3c4ccccc4c4c5cccc6c5n(c43)-c3ccccc3S6)n2)cc1 ; c1ccc(-c2nc(-c3ccc4ccccc4c3)nc(-c3ccccc3-n3c4ccccc4c4c5cccc6c5n(c43)-c3ccccc3S6)n2)cc1]

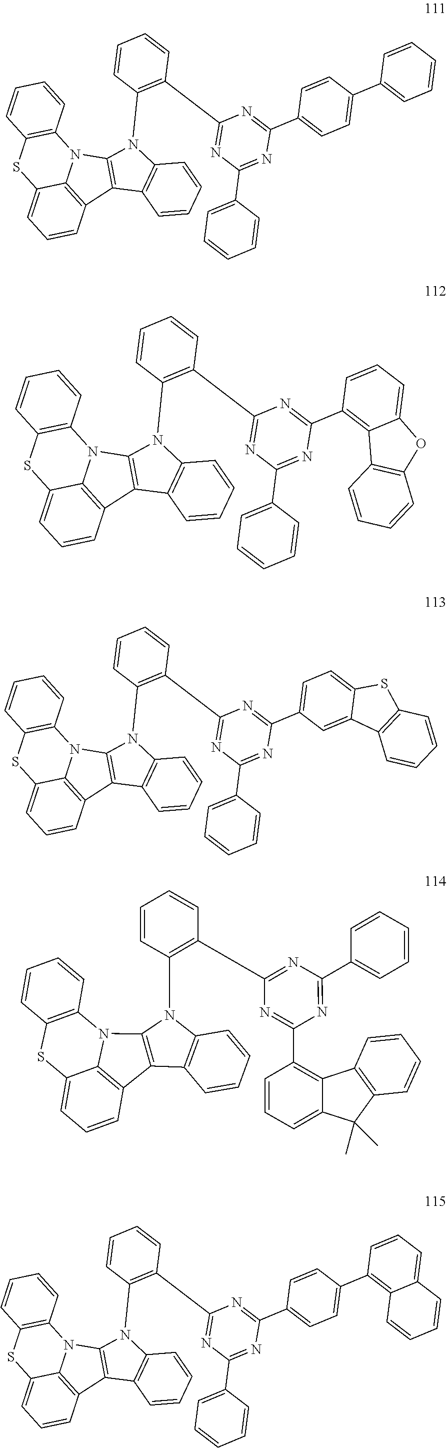 [SMILES: c1ccc(-c2ccc(-c3nc(-c4ccccc4)nc(-c4ccccc4-n4c5ccccc5c5c6cccc7c6n(c54)-c4ccccc4S7)n3)cc2)cc1 ; c1ccc(-c2nc(-c3ccccc3-n3c4ccccc4c4c5cccc6c5n(c43)-c3ccccc3S6)nc(-c3cccc4oc5ccccc5c34)n2)cc1 ; c1ccc(-c2nc(-c3ccc4sc5ccccc5c4c3)nc(-c3ccccc3-n3c4ccccc4c4c5cccc6c5n(c43)-c3ccccc3S6)n2)cc1 ; CC1(C)c2ccccc2-c2c(-c3nc(-c4ccccc4)nc(-c4ccccc4-n4c5ccccc5c5c6cccc7c6n(c54)-c4ccccc4S7)n3)cccc21 ; c1ccc(-c2nc(-c3ccc(-c4cccc5ccccc45)cc3)nc(-c3ccccc3-n3c4ccccc4c4c5cccc6c5n(c43)-c3ccccc3S6)n2)cc1]

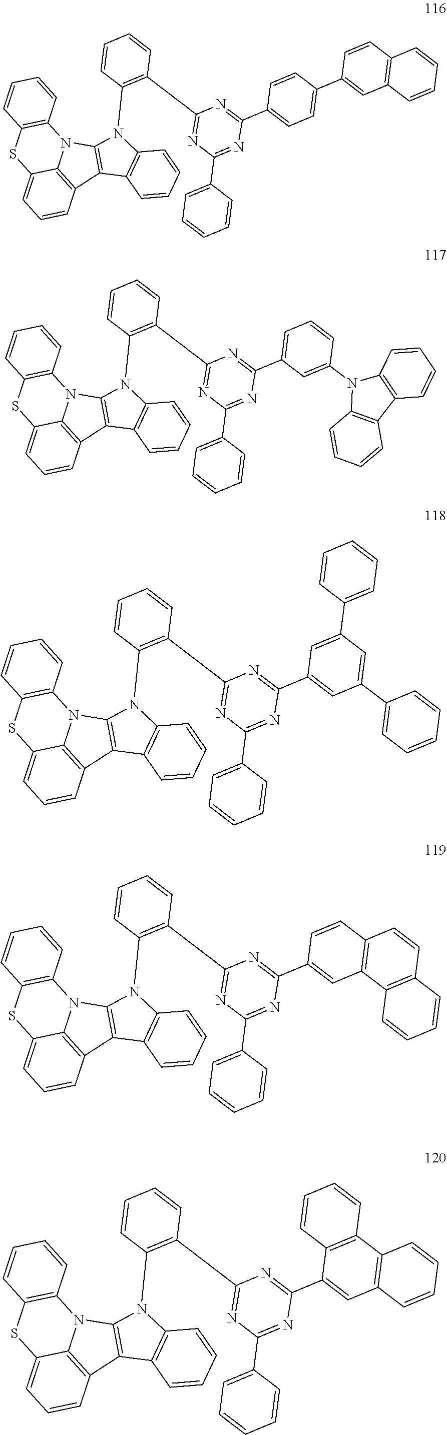 [SMILES: c1ccc(-c2nc(-c3ccc(-c4ccc5ccccc5c4)cc3)nc(-c3ccccc3-n3c4ccccc4c4c5cccc6c5n(c43)-c3ccccc3S6)n2)cc1 ; c1ccc(-c2nc(-c3cccc(-n4c5ccccc5c5ccccc54)c3)nc(-c3ccccc3-n3c4ccccc4c4c5cccc6c5n(c43)-c3ccccc3S6)n2)cc1 ; c1ccc(-c2cc(-c3ccccc3)cc(-c3nc(-c4ccccc4)nc(-c4ccccc4-n4c5ccccc5c5c6cccc7c6n(c54)-c4ccccc4S7)n3)c2)cc1 ; c1ccc(-c2nc(-c3ccc4ccc5ccccc5c4c3)nc(-c3ccccc3-n3c4ccccc4c4c5cccc6c5n(c43)-c3ccccc3S6)n2)cc1 ; c1ccc(-c2nc(-c3ccccc3-n3c4ccccc4c4c5cccc6c5n(c43)-c3ccccc3S6)nc(-c3cc4ccccc4c4ccccc34)n2)cc1]

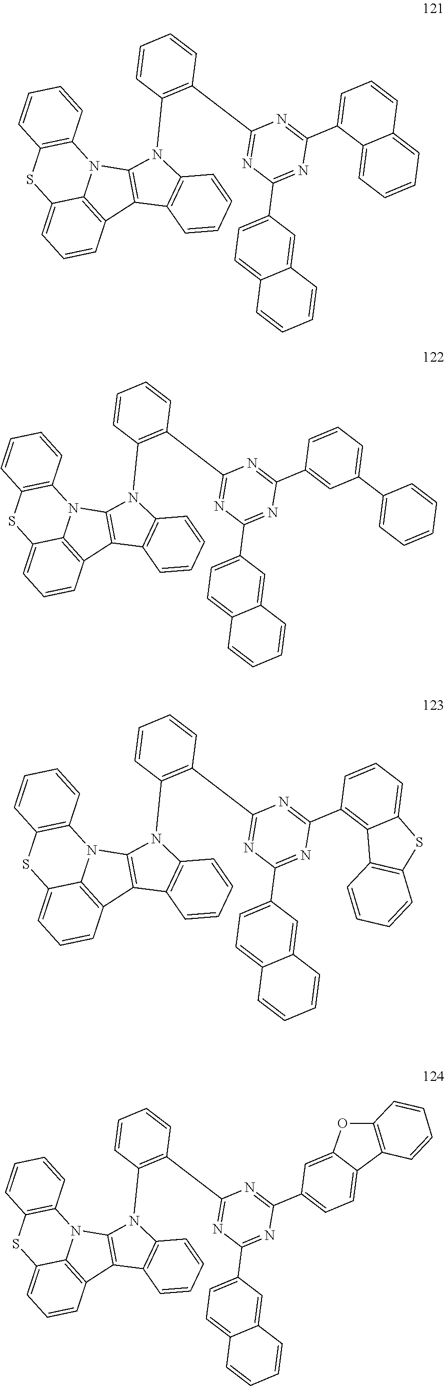 [SMILES: c1ccc2c(c1)Sc1cccc3c4c5ccccc5n(-c5ccccc5-c5nc(-c6ccc7ccccc7c6)nc(-c6cccc7ccccc67)n5)c4n-2c13 ; c1ccc(-c2cccc(-c3nc(-c4ccc5ccccc5c4)nc(-c4ccccc4-n4c5ccccc5c5c6cccc7c6n(c54)-c4ccccc4S7)n3)c2)cc1 ; c1ccc2c(c1)Sc1cccc3c4c5ccccc5n(-c5ccccc5-c5nc(-c6ccc7ccccc7c6)nc(-c6cccc7sc8ccccc8c67)n5)c4n-2c13 ; c1ccc2c(c1)Sc1cccc3c4c5ccccc5n(-c5ccccc5-c5nc(-c6ccc7ccccc7c6)nc(-c6ccc7c(c6)oc6ccccc67)n5)c4n-2c13]

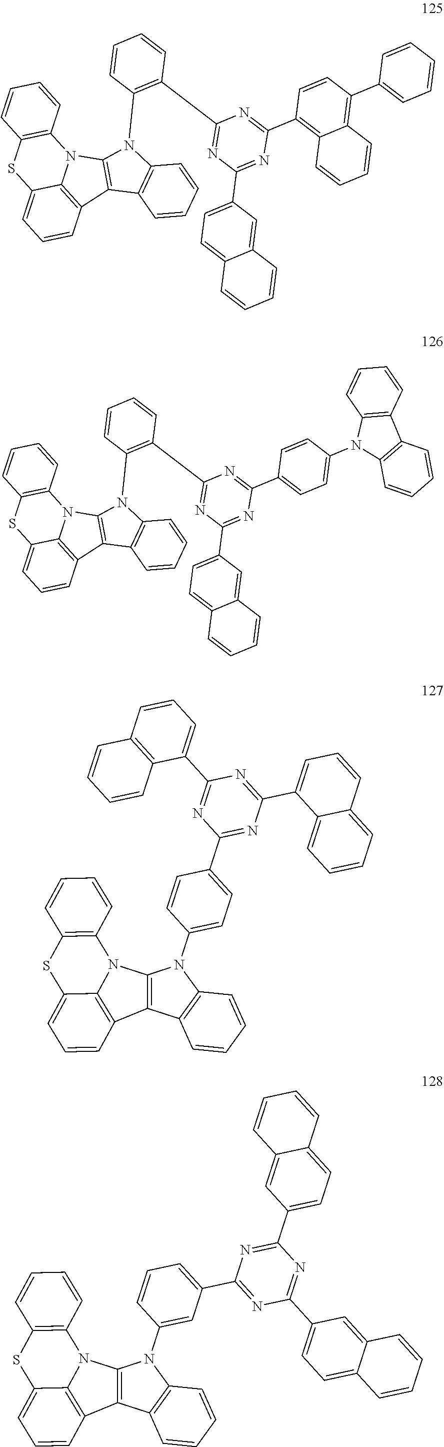 [SMILES: c1ccc(-c2ccc(-c3nc(-c4ccc5ccccc5c4)nc(-c4ccccc4-n4c5ccccc5c5c6cccc7c6n(c54)-c4ccccc4S7)n3)c3ccccc23)cc1 ; c1ccc2c(c1)Sc1cccc3c4c5ccccc5n(-c5ccccc5-c5nc(-c6ccc(-n7c8ccccc8c8ccccc87)cc6)nc(-c6ccc7ccccc7c6)n5)c4n-2c13 ; c1ccc2c(c1)Sc1cccc3c4c5ccccc5n(-c5ccc(-c6nc(-c7cccc8ccccc78)nc(-c7cccc8ccccc78)n6)cc5)c4n-2c13 ; c1cc(-c2nc(-c3ccc4ccccc4c3)nc(-c3ccc4ccccc4c3)n2)cc(-n2c3ccccc3c3c4cccc5c4n(c32)-c2ccccc2S5)c1]

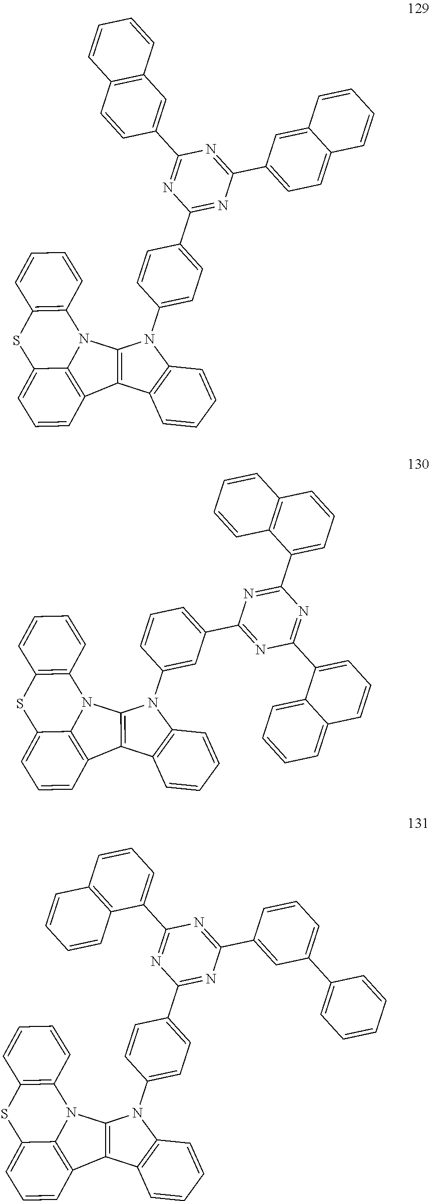 [SMILES: c1ccc2c(c1)Sc1cccc3c4c5ccccc5n(-c5ccc(-c6nc(-c7ccc8ccccc8c7)nc(-c7ccc8ccccc8c7)n6)cc5)c4n-2c13 ; c1cc(-c2nc(-c3cccc4ccccc34)nc(-c3cccc4ccccc34)n2)cc(-n2c3ccccc3c3c4cccc5c4n(c32)-c2ccccc2S5)c1 ; c1ccc(-c2cccc(-c3nc(-c4ccc(-n5c6ccccc6c6c7cccc8c7n(c65)-c5ccccc5S8)cc4)nc(-c4cccc5ccccc45)n3)c2)cc1]

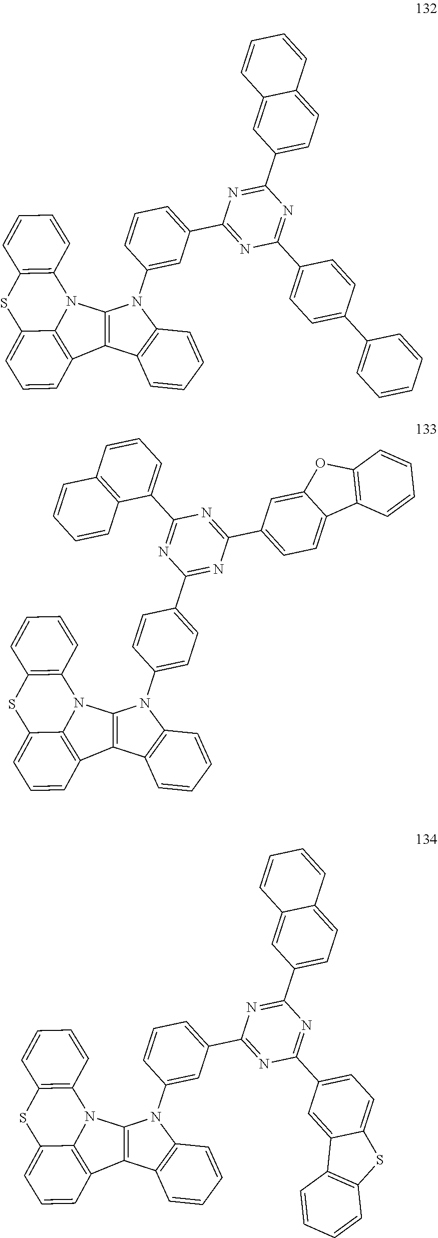 [SMILES: c1ccc(-c2ccc(-c3nc(-c4cccc(-n5c6ccccc6c6c7cccc8c7n(c65)-c5ccccc5S8)c4)nc(-c4ccc5ccccc5c4)n3)cc2)cc1 ; c1ccc2c(c1)Sc1cccc3c4c5ccccc5n(-c5ccc(-c6nc(-c7ccc8c(c7)oc7ccccc78)nc(-c7cccc8ccccc78)n6)cc5)c4n-2c13 ; c1cc(-c2nc(-c3ccc4ccccc4c3)nc(-c3ccc4sc5ccccc5c4c3)n2)cc(-n2c3ccccc3c3c4cccc5c4n(c32)-c2ccccc2S5)c1]

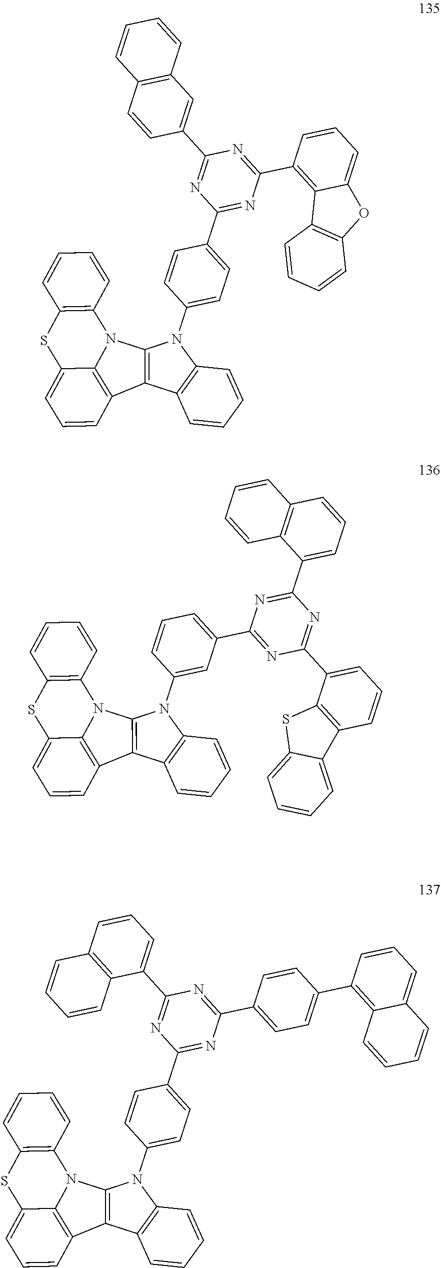 [SMILES: c1ccc2c(c1)Sc1cccc3c4c5ccccc5n(-c5ccc(-c6nc(-c7ccc8ccccc8c7)nc(-c7cccc8oc9ccccc9c78)n6)cc5)c4n-2c13 ; c1cc(-c2nc(-c3cccc4ccccc34)nc(-c3cccc4c3sc3ccccc34)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc(-c8cccc9ccccc89)cc7)nc(-c7cccc8ccccc78)n6)cc5)c4n-2c13]

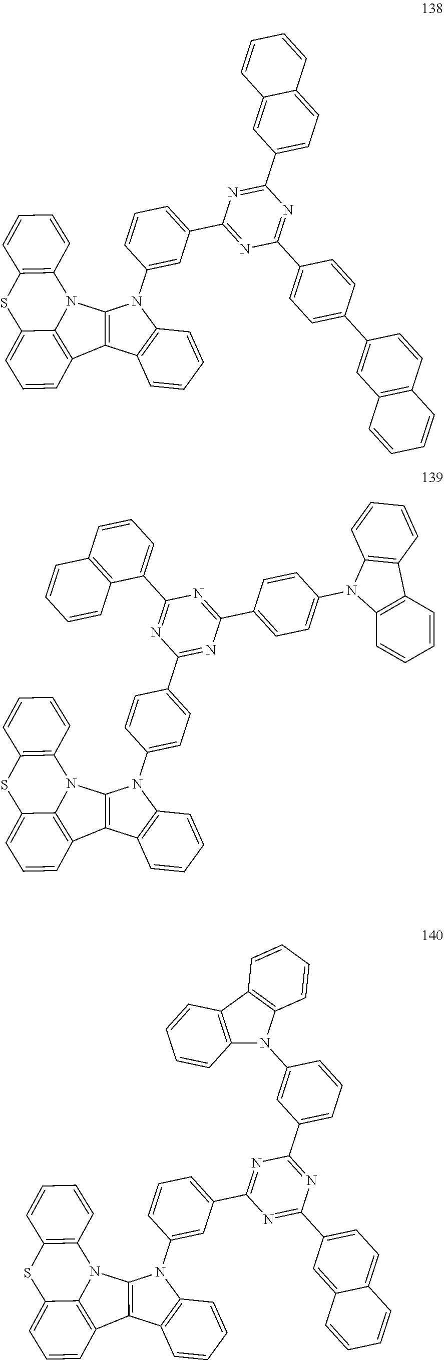 [SMILES: c1cc(-c2nc(-c3ccc(-c4ccc5ccccc5c4)cc3)nc(-c3ccc4ccccc4c3)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc(-n8c9ccccc9c9ccccc98)cc7)nc(-c7cccc8ccccc78)n6)cc5)c4n-2c13 ; c1cc(-c2nc(-c3cccc(-n4c5ccccc5c5c6cccc7c6n(c54)-c4ccccc4S7)c3)nc(-c3ccc4ccccc4c3)n2)cc(-n2c3ccccc3c3ccccc32)c1]

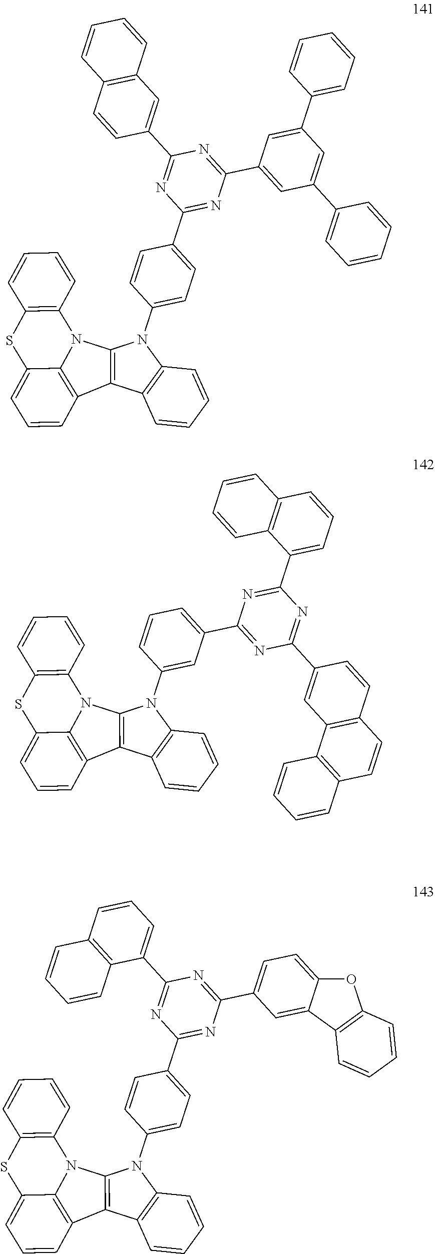 [SMILES: c1ccc(-c2cc(-c3ccccc3)cc(-c3nc(-c4ccc(-n5c6ccccc6c6c7cccc8c7n(c65)-c5ccccc5S8)cc4)nc(-c4ccc5ccccc5c4)n3)c2)cc1 ; c1cc(-c2nc(-c3ccc4ccc5ccccc5c4c3)nc(-c3cccc4ccccc34)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc8oc9ccccc9c8c7)nc(-c7cccc8ccccc78)n6)cc5)c4n-2c13]

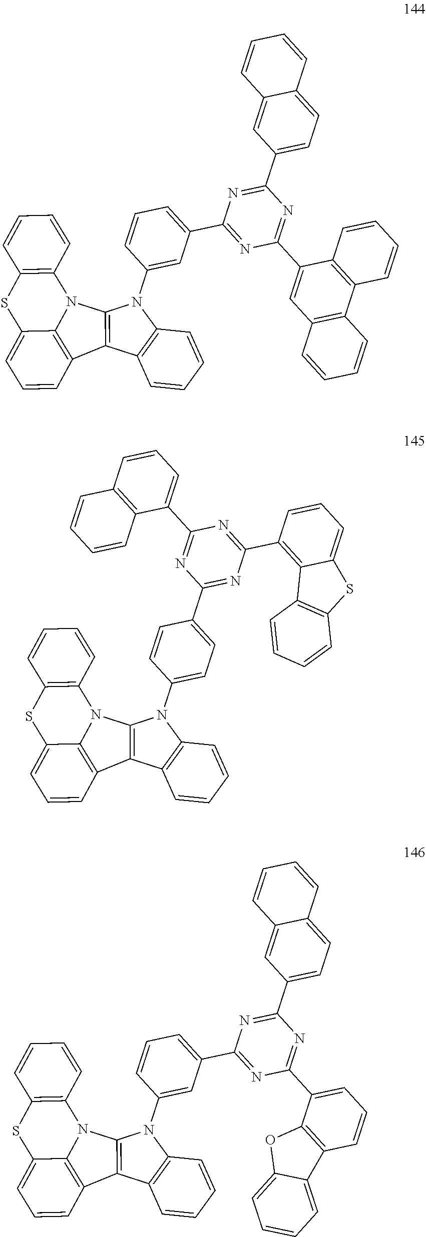 [SMILES: c1cc(-c2nc(-c3ccc4ccccc4c3)nc(-c3cc4ccccc4c4ccccc34)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7cccc8ccccc78)nc(-c7cccc8sc9ccccc9c78)n6)cc5)c4n-2c13 ; c1cc(-c2nc(-c3ccc4ccccc4c3)nc(-c3cccc4c3oc3ccccc34)n2)cc(-n2c3ccccc3c3c4cccc5c4n(c32)-c2ccccc2S5)c1]

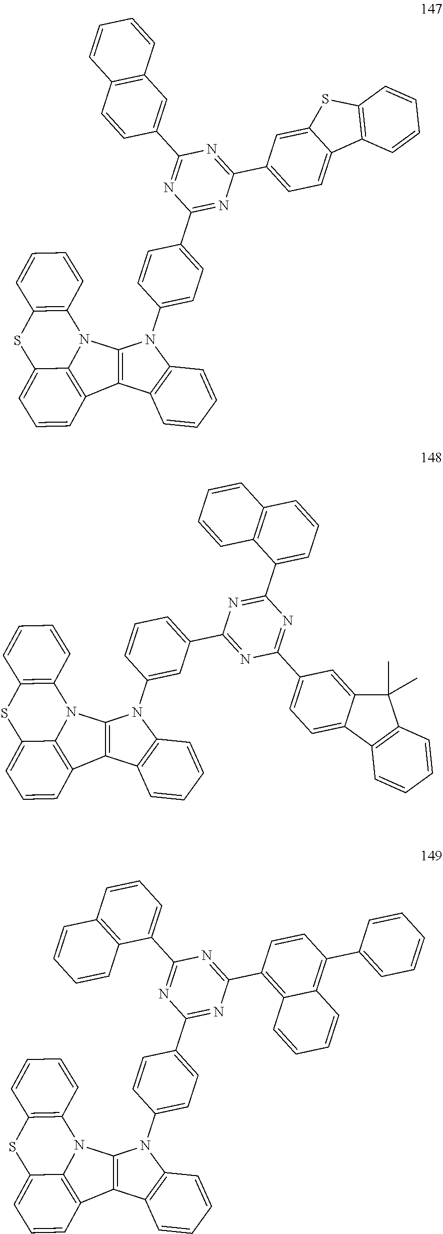 [SMILES: c1ccc2c(c1)Sc1cccc3c4c5ccccc5n(-c5ccc(-c6nc(-c7ccc8ccccc8c7)nc(-c7ccc8c(c7)sc7ccccc78)n6)cc5)c4n-2c13 ; CC1(C)c2ccccc2-c2ccc(-c3nc(-c4cccc(-n5c6ccccc6c6c7cccc8c7n(c65)-c5ccccc5S8)c4)nc(-c4cccc5ccccc45)n3)cc21 ; c1ccc(-c2ccc(-c3nc(-c4ccc(-n5c6ccccc6c6c7cccc8c7n(c65)-c5ccccc5S8)cc4)nc(-c4cccc5ccccc45)n3)c3ccccc23)cc1]

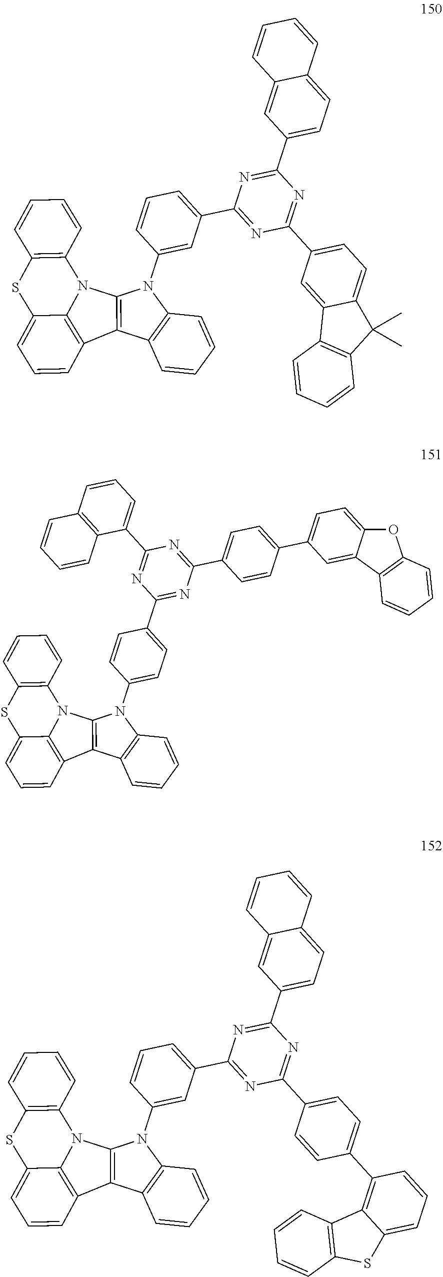 [SMILES: CC1(C)c2ccccc2-c2cc(-c3nc(-c4cccc(-n5c6ccccc6c6c7cccc8c7n(c65)-c5ccccc5S8)c4)nc(-c4ccc5ccccc5c4)n3)ccc21 ; c1ccc2c(c1)Sc1cccc3c4c5ccccc5n(-c5ccc(-c6nc(-c7ccc(-c8ccc9oc%10ccccc%10c9c8)cc7)nc(-c7cccc8ccccc78)n6)cc5)c4n-2c13 ; c1cc(-c2nc(-c3ccc(-c4cccc5sc6ccccc6c45)cc3)nc(-c3ccc4ccccc4c3)n2)cc(-n2c3ccccc3c3c4cccc5c4n(c32)-c2ccccc2S5)c1]

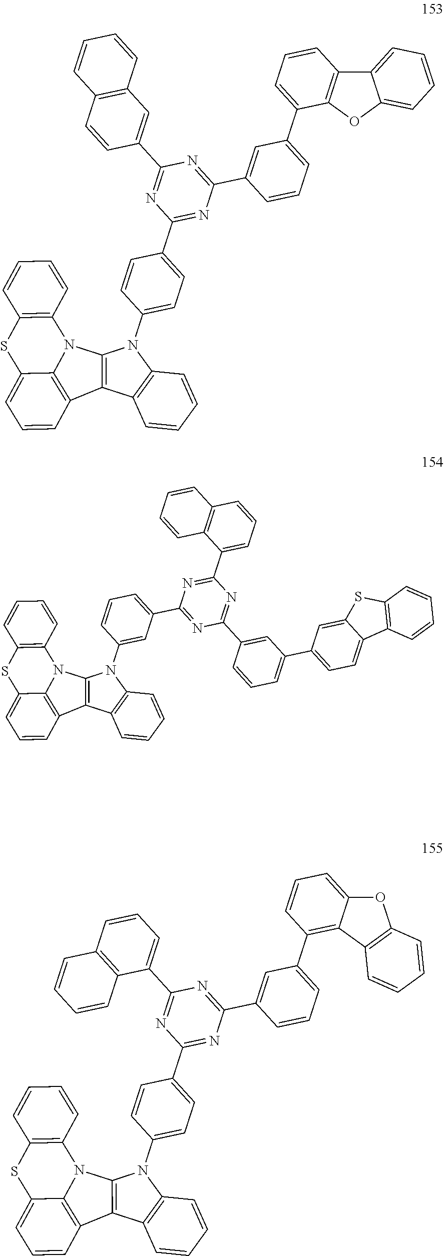 [SMILES: c1cc(-c2nc(-c3ccc(-n4c5ccccc5c5c6cccc7c6n(c54)-c4ccccc4S7)cc3)nc(-c3ccc4ccccc4c3)n2)cc(-c2cccc3c2oc2ccccc23)c1 ; c1cc(-c2ccc3c(c2)sc2ccccc23)cc(-c2nc(-c3cccc(-n4c5ccccc5c5c6cccc7c6n(c54)-c4ccccc4S7)c3)nc(-c3cccc4ccccc34)n2)c1 ; c1cc(-c2nc(-c3ccc(-n4c5ccccc5c5c6cccc7c6n(c54)-c4ccccc4S7)cc3)nc(-c3cccc4ccccc34)n2)cc(-c2cccc3oc4ccccc4c23)c1]

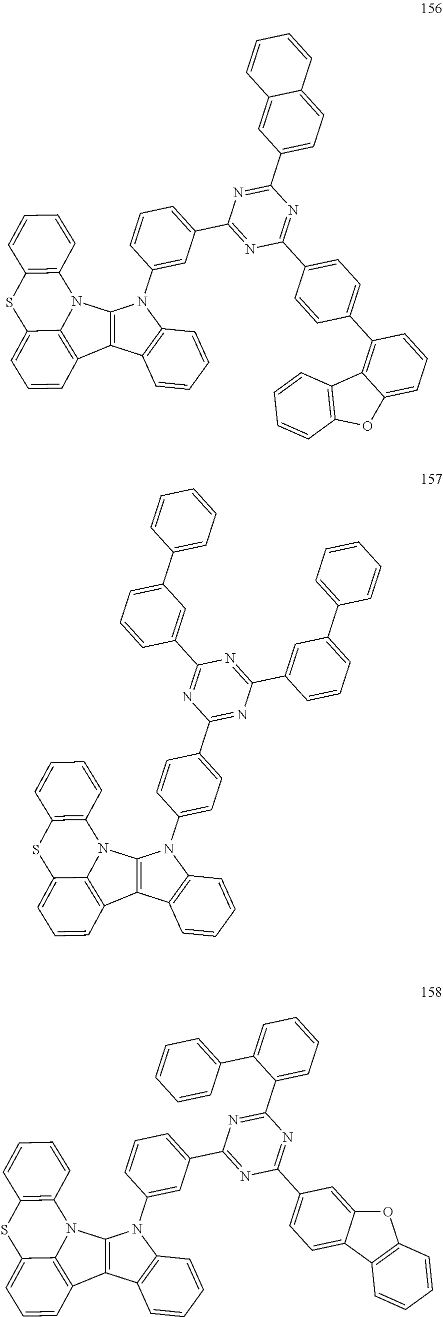 [SMILES: c1cc(-c2nc(-c3ccc(-c4cccc5oc6ccccc6c45)cc3)nc(-c3ccc4ccccc4c3)n2)cc(-n2c3ccccc3c3c4cccc5c4n(c32)-c2ccccc2S5)c1 ; c1ccc(-c2cccc(-c3nc(-c4ccc(-n5c6ccccc6c6c7cccc8c7n(c65)-c5ccccc5S8)cc4)nc(-c4cccc(-c5ccccc5)c4)n3)c2)cc1 ; c1ccc(-c2ccccc2-c2nc(-c3cccc(-n4c5ccccc5c5c6cccc7c6n(c54)-c4ccccc4S7)c3)nc(-c3ccc4c(c3)oc3ccccc34)n2)cc1]

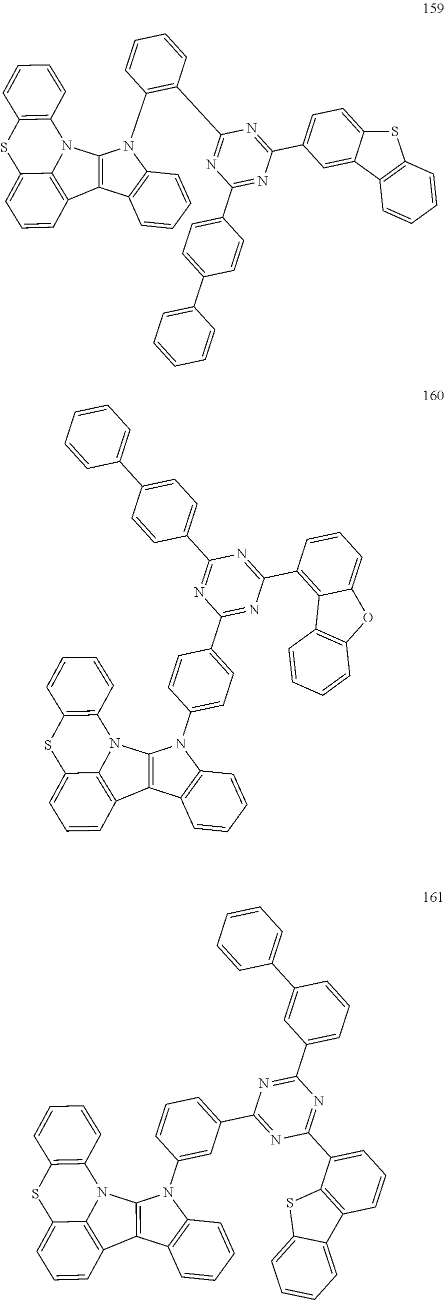 [SMILES: c1ccc(-c2ccc(-c3nc(-c4ccc5sc6ccccc6c5c4)nc(-c4ccccc4-n4c5ccccc5c5c6cccc7c6n(c54)-c4ccccc4S7)n3)cc2)cc1 ; c1ccc(-c2ccc(-c3nc(-c4ccc(-n5c6ccccc6c6c7cccc8c7n(c65)-c5ccccc5S8)cc4)nc(-c4cccc5oc6ccccc6c45)n3)cc2)cc1 ; c1ccc(-c2cccc(-c3nc(-c4cccc(-n5c6ccccc6c6c7cccc8c7n(c65)-c5ccccc5S8)c4)nc(-c4cccc5c4sc4ccccc45)n3)c2)cc1]

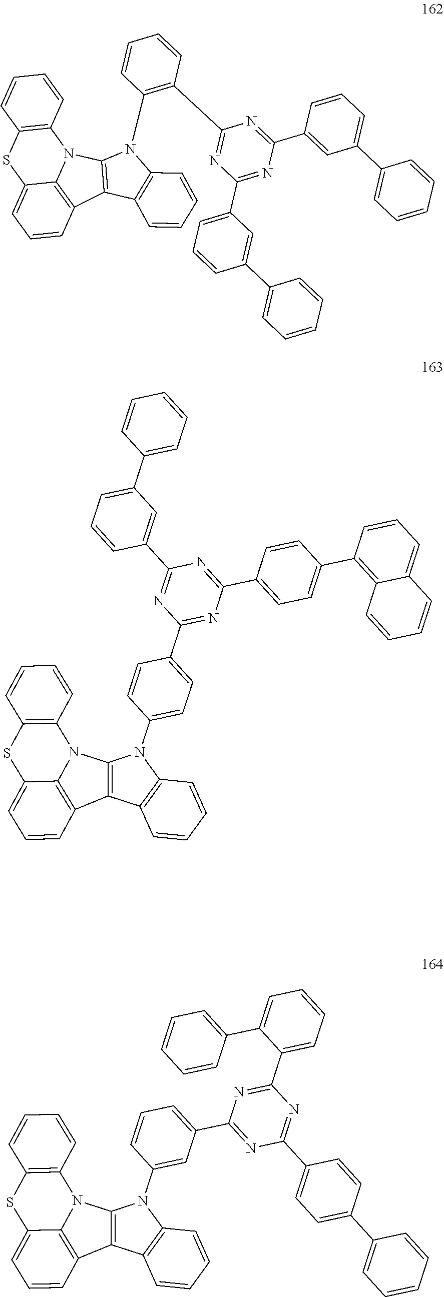 [SMILES: c1ccc(-c2cccc(-c3nc(-c4cccc(-c5ccccc5)c4)nc(-c4ccccc4-n4c5ccccc5c5c6cccc7c6n(c54)-c4ccccc4S7)n3)c2)cc1 ; c1ccc(-c2cccc(-c3nc(-c4ccc(-c5cccc6ccccc56)cc4)nc(-c4ccc(-n5c6ccccc6c6c7cccc8c7n(c65)-c5ccccc5S8)cc4)n3)c2)cc1 ; c1ccc(-c2ccc(-c3nc(-c4cccc(-n5c6ccccc6c6c7cccc8c7n(c65)-c5ccccc5S8)c4)nc(-c4ccccc4-c4ccccc4)n3)cc2)cc1]

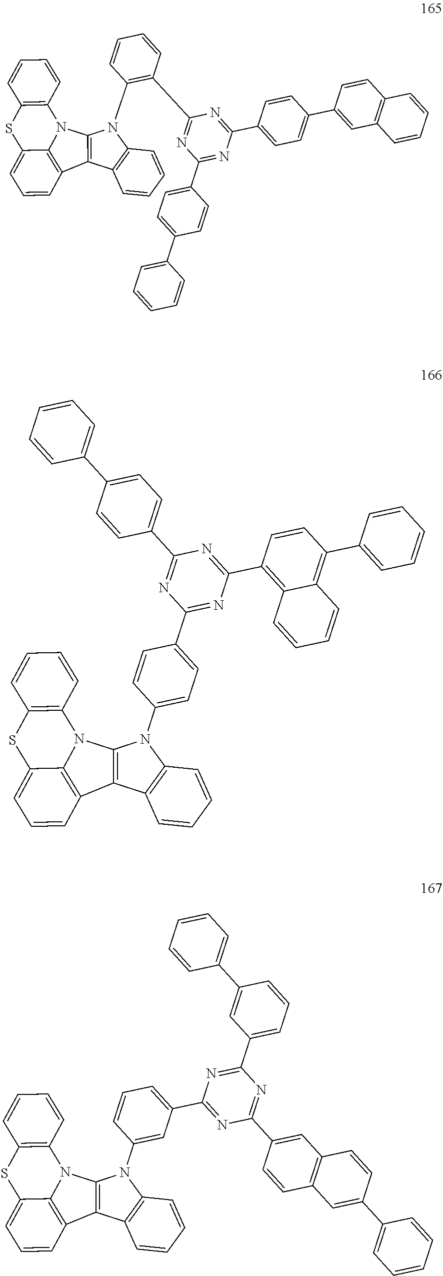 [SMILES: c1ccc(-c2ccc(-c3nc(-c4ccc(-c5ccc6ccccc6c5)cc4)nc(-c4ccccc4-n4c5ccccc5c5c6cccc7c6n(c54)-c4ccccc4S7)n3)cc2)cc1 ; c1ccc(-c2ccc(-c3nc(-c4ccc(-n5c6ccccc6c6c7cccc8c7n(c65)-c5ccccc5S8)cc4)nc(-c4ccc(-c5ccccc5)c5ccccc45)n3)cc2)cc1 ; c1ccc(-c2cccc(-c3nc(-c4cccc(-n5c6ccccc6c6c7cccc8c7n(c65)-c5ccccc5S8)c4)nc(-c4ccc5cc(-c6ccccc6)ccc5c4)n3)c2)cc1]

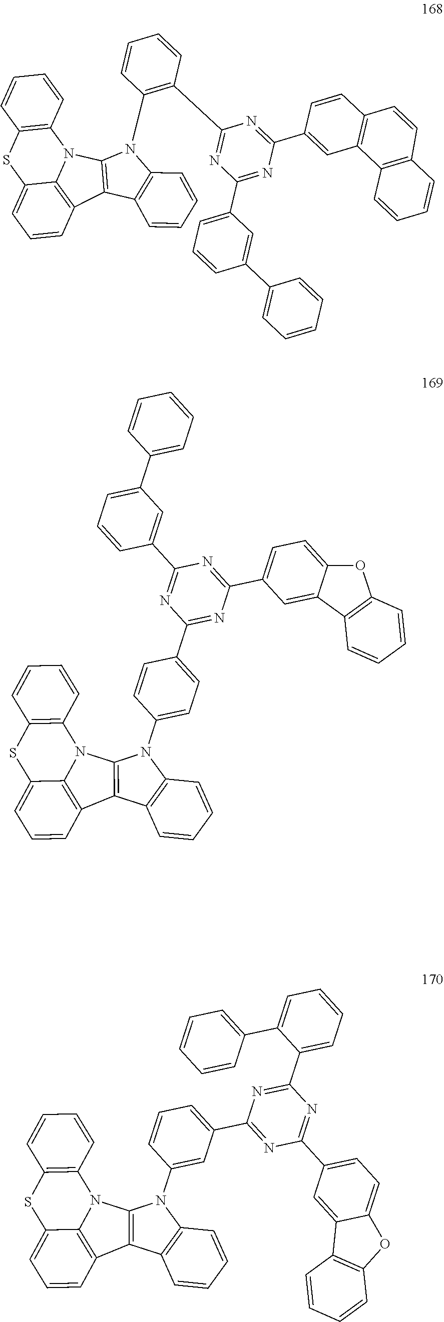 [SMILES: c1ccc(-c2cccc(-c3nc(-c4ccc5ccc6ccccc6c5c4)nc(-c4ccccc4-n4c5ccccc5c5c6cccc7c6n(c54)-c4ccccc4S7)n3)c2)cc1 ; c1ccc(-c2cccc(-c3nc(-c4ccc(-n5c6ccccc6c6c7cccc8c7n(c65)-c5ccccc5S8)cc4)nc(-c4ccc5oc6ccccc6c5c4)n3)c2)cc1 ; c1ccc(-c2ccccc2-c2nc(-c3cccc(-n4c5ccccc5c5c6cccc7c6n(c54)-c4ccccc4S7)c3)nc(-c3ccc4oc5ccccc5c4c3)n2)cc1]

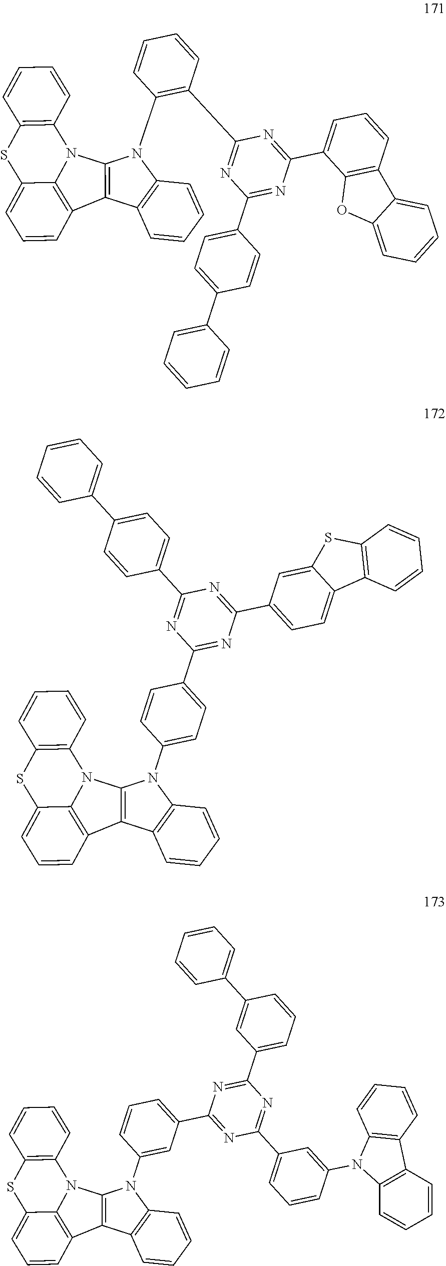 [SMILES: c1ccc(-c2ccc(-c3nc(-c4ccccc4-n4c5ccccc5c5c6cccc7c6n(c54)-c4ccccc4S7)nc(-c4cccc5c4oc4ccccc45)n3)cc2)cc1 ; c1ccc(-c2ccc(-c3nc(-c4ccc(-n5c6ccccc6c6c7cccc8c7n(c65)-c5ccccc5S8)cc4)nc(-c4ccc5c(c4)sc4ccccc45)n3)cc2)cc1 ; c1ccc(-c2cccc(-c3nc(-c4cccc(-n5c6ccccc6c6ccccc65)c4)nc(-c4cccc(-n5c6ccccc6c6c7cccc8c7n(c65)-c5ccccc5S8)c4)n3)c2)cc1]

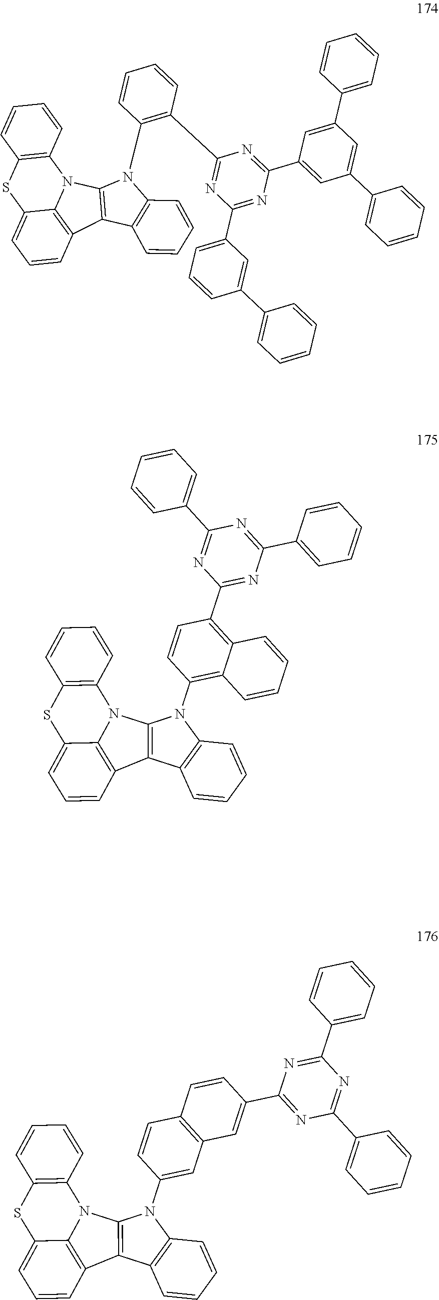 [SMILES: c1ccc(-c2cccc(-c3nc(-c4cc(-c5ccccc5)cc(-c5ccccc5)c4)nc(-c4ccccc4-n4c5ccccc5c5c6cccc7c6n(c54)-c4ccccc4S7)n3)c2)cc1 ; c1ccc(-c2nc(-c3ccccc3)nc(-c3ccc(-n4c5ccccc5c5c6cccc7c6n(c54)-c4ccccc4S7)c4ccccc34)n2)cc1 ; c1ccc(-c2nc(-c3ccccc3)nc(-c3ccc4ccc(-n5c6ccccc6c6c7cccc8c7n(c65)-c5ccccc5S8)cc4c3)n2)cc1]

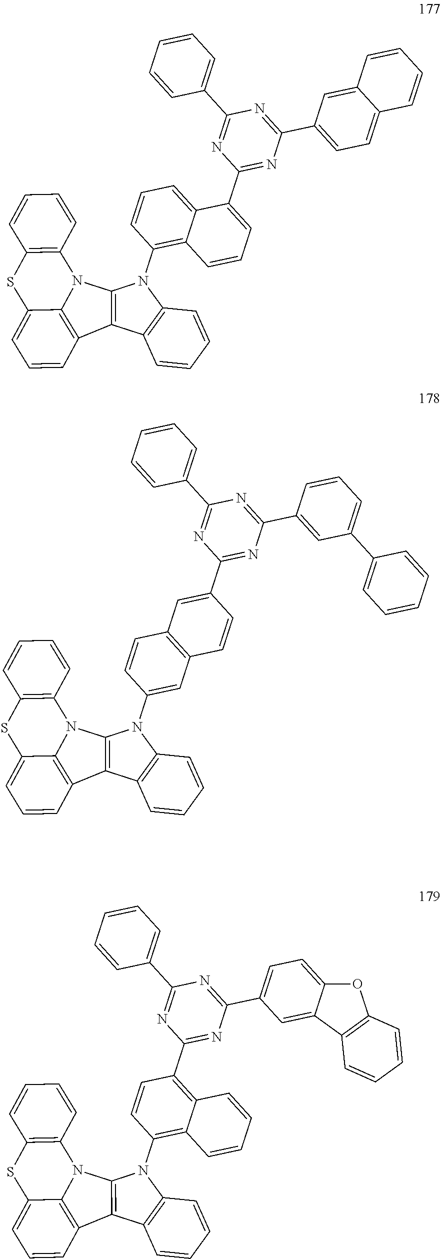 [SMILES: c1ccc(-c2nc(-c3ccc4ccccc4c3)nc(-c3cccc4c(-n5c6ccccc6c6c7cccc8c7n(c65)-c5ccccc5S8)cccc34)n2)cc1 ; c1ccc(-c2cccc(-c3nc(-c4ccccc4)nc(-c4ccc5cc(-n6c7ccccc7c7c8cccc9c8n(c76)-c6ccccc6S9)ccc5c4)n3)c2)cc1 ; c1ccc(-c2nc(-c3ccc4oc5ccccc5c4c3)nc(-c3ccc(-n4c5ccccc5c5c6cccc7c6n(c54)-c4ccccc4S7)c4ccccc34)n2)cc1]

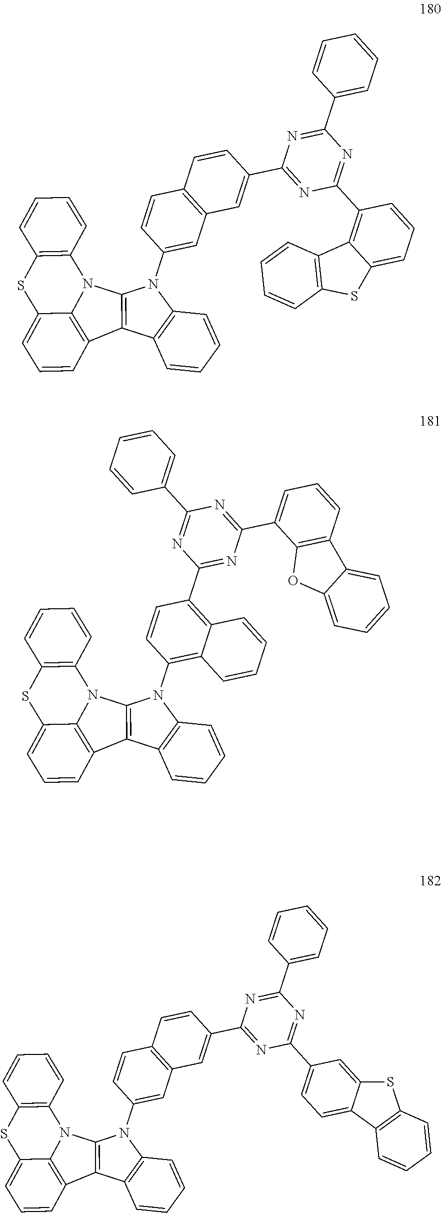 [SMILES: c1ccc(-c2nc(-c3ccc4ccc(-n5c6ccccc6c6c7cccc8c7n(c65)-c5ccccc5S8)cc4c3)nc(-c3cccc4sc5ccccc5c34)n2)cc1 ; c1ccc(-c2nc(-c3ccc(-n4c5ccccc5c5c6cccc7c6n(c54)-c4ccccc4S7)c4ccccc34)nc(-c3cccc4c3oc3ccccc34)n2)cc1 ; c1ccc(-c2nc(-c3ccc4ccc(-n5c6ccccc6c6c7cccc8c7n(c65)-c5ccccc5S8)cc4c3)nc(-c3ccc4c(c3)sc3ccccc34)n2)cc1]

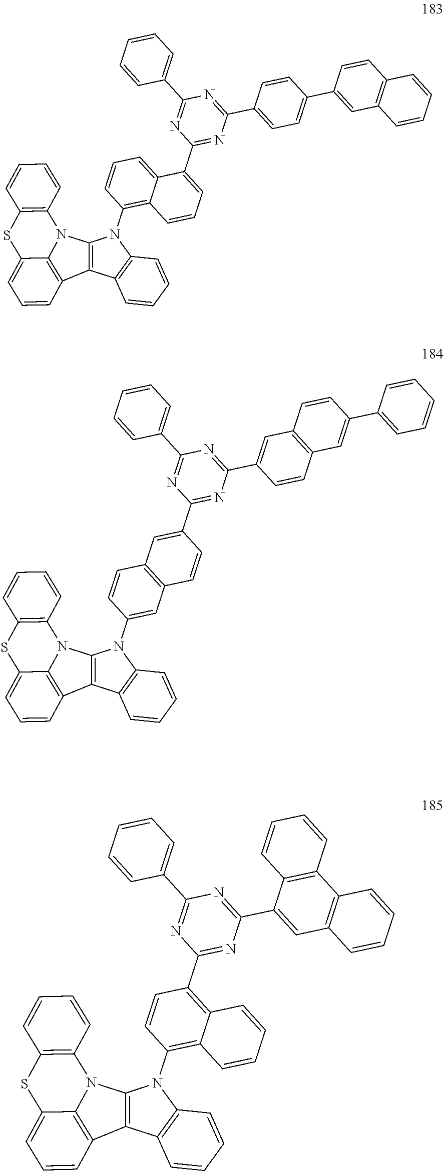 [SMILES: c1ccc(-c2nc(-c3ccc(-c4ccc5ccccc5c4)cc3)nc(-c3cccc4c(-n5c6ccccc6c6c7cccc8c7n(c65)-c5ccccc5S8)cccc34)n2)cc1 ; c1ccc(-c2ccc3cc(-c4nc(-c5ccccc5)nc(-c5ccc6cc(-n7c8ccccc8c8c9cccc%10c9n(c87)-c7ccccc7S%10)ccc6c5)n4)ccc3c2)cc1 ; c1ccc(-c2nc(-c3ccc(-n4c5ccccc5c5c6cccc7c6n(c54)-c4ccccc4S7)c4ccccc34)nc(-c3cc4ccccc4c4ccccc34)n2)cc1]

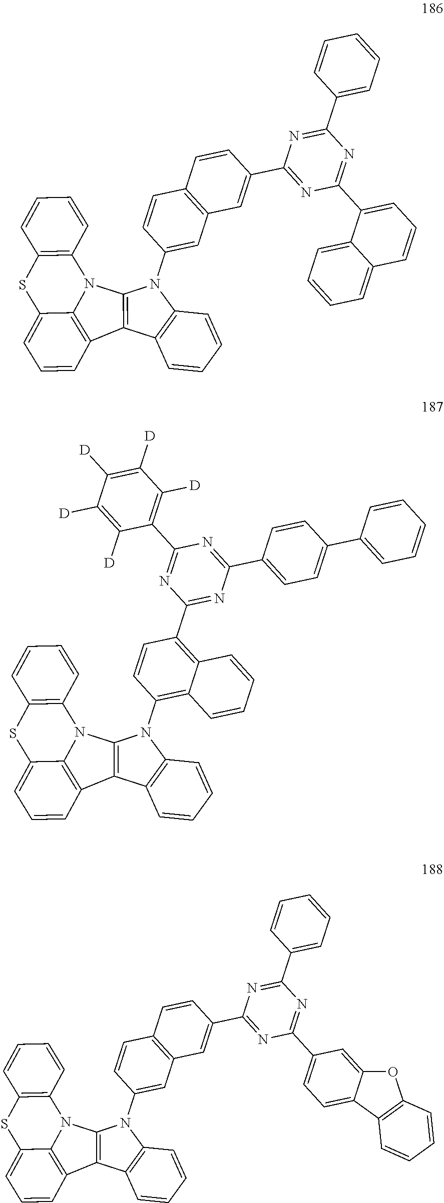 [SMILES: c1ccc(-c2nc(-c3ccc4ccc(-n5c6ccccc6c6c7cccc8c7n(c65)-c5ccccc5S8)cc4c3)nc(-c3cccc4ccccc34)n2)cc1 ; [2H]c1c([2H])c([2H])c(-c2nc(-c3ccc(-c4ccccc4)cc3)nc(-c3ccc(-n4c5ccccc5c5c6cccc7c6n(c54)-c4ccccc4S7)c4ccccc34)n2)c([2H])c1[2H] ; c1ccc(-c2nc(-c3ccc4ccc(-n5c6ccccc6c6c7cccc8c7n(c65)-c5ccccc5S8)cc4c3)nc(-c3ccc4c(c3)oc3ccccc34)n2)cc1]

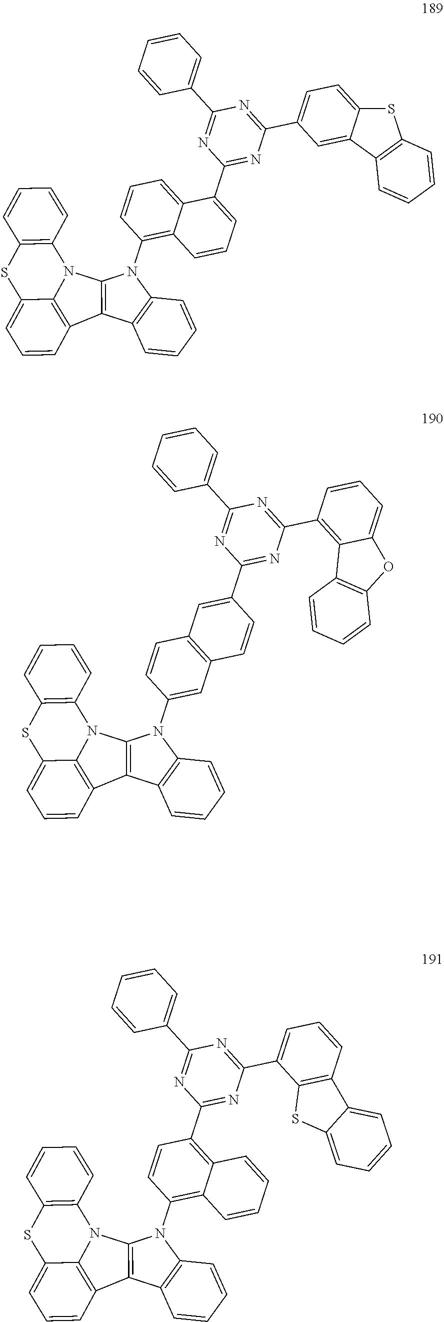 [SMILES: c1ccc(-c2nc(-c3ccc4sc5ccccc5c4c3)nc(-c3cccc4c(-n5c6ccccc6c6c7cccc8c7n(c65)-c5ccccc5S8)cccc34)n2)cc1 ; c1ccc(-c2nc(-c3ccc4cc(-n5c6ccccc6c6c7cccc8c7n(c65)-c5ccccc5S8)ccc4c3)nc(-c3cccc4oc5ccccc5c34)n2)cc1 ; c1ccc(-c2nc(-c3ccc(-n4c5ccccc5c5c6cccc7c6n(c54)-c4ccccc4S7)c4ccccc34)nc(-c3cccc4c3sc3ccccc34)n2)cc1]

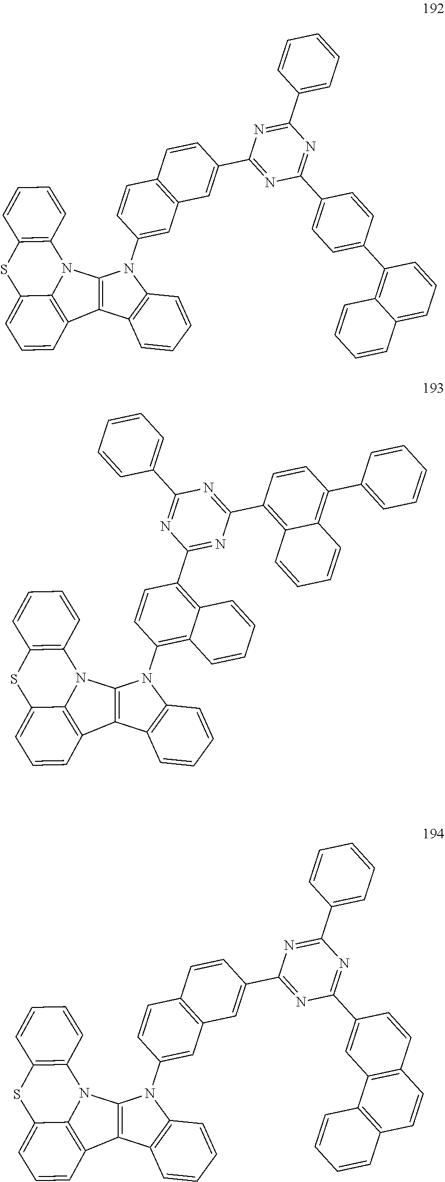 [SMILES: c1ccc(-c2nc(-c3ccc(-c4cccc5ccccc45)cc3)nc(-c3ccc4ccc(-n5c6ccccc6c6c7cccc8c7n(c65)-c5ccccc5S8)cc4c3)n2)cc1 ; c1ccc(-c2nc(-c3ccc(-c4ccccc4)c4ccccc34)nc(-c3ccc(-n4c5ccccc5c5c6cccc7c6n(c54)-c4ccccc4S7)c4ccccc34)n2)cc1 ; c1ccc(-c2nc(-c3ccc4ccc(-n5c6ccccc6c6c7cccc8c7n(c65)-c5ccccc5S8)cc4c3)nc(-c3ccc4ccc5ccccc5c4c3)n2)cc1]

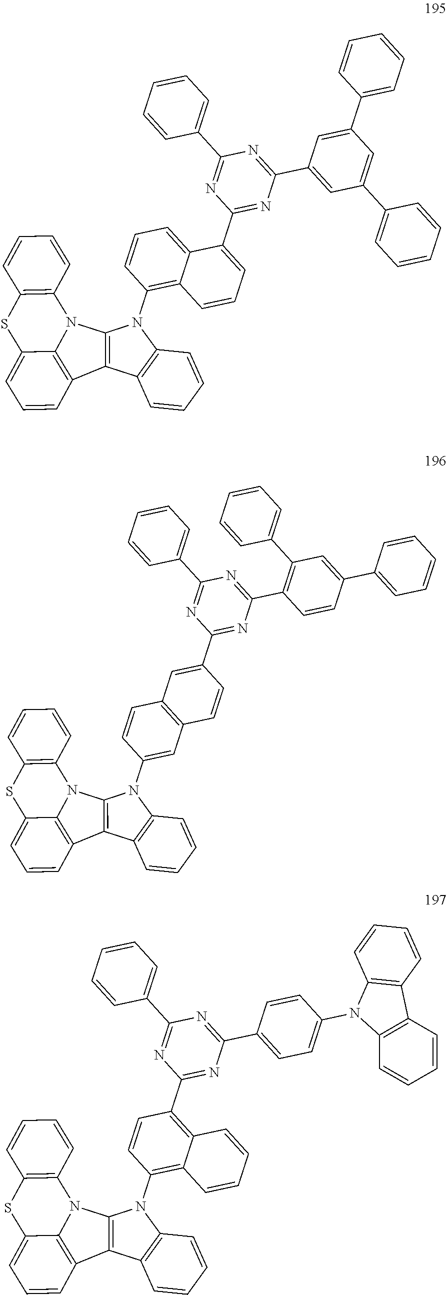 [SMILES: c1ccc(-c2cc(-c3ccccc3)cc(-c3nc(-c4ccccc4)nc(-c4cccc5c(-n6c7ccccc7c7c8cccc9c8n(c76)-c6ccccc6S9)cccc45)n3)c2)cc1 ; c1ccc(-c2ccc(-c3nc(-c4ccccc4)nc(-c4ccc5cc(-n6c7ccccc7c7c8cccc9c8n(c76)-c6ccccc6S9)ccc5c4)n3)c(-c3ccccc3)c2)cc1 ; c1ccc(-c2nc(-c3ccc(-n4c5ccccc5c5ccccc54)cc3)nc(-c3ccc(-n4c5ccccc5c5c6cccc7c6n(c54)-c4ccccc4S7)c4ccccc34)n2)cc1]

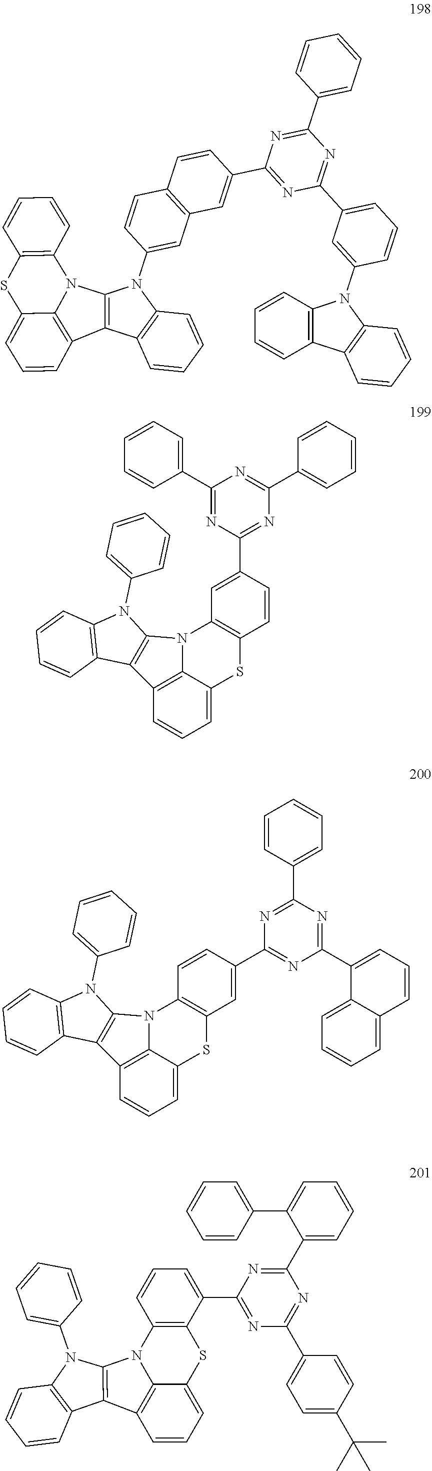 [SMILES: c1ccc(-c2nc(-c3cccc(-n4c5ccccc5c5ccccc54)c3)nc(-c3ccc4ccc(-n5c6ccccc6c6c7cccc8c7n(c65)-c5ccccc5S8)cc4c3)n2)cc1 ; c1ccc(-c2nc(-c3ccccc3)nc(-c3ccc4c(c3)-n3c5c(cccc5c5c6ccccc6n(-c6ccccc6)c53)S4)n2)cc1 ; c1ccc(-c2nc(-c3ccc4c(c3)Sc3cccc5c6c7ccccc7n(-c7ccccc7)c6n-4c35)nc(-c3cccc4ccccc34)n2)cc1 ; CC(C)(C)c1ccc(-c2nc(-c3ccccc3-c3ccccc3)nc(-c3cccc4c3Sc3cccc5c6c7ccccc7n(-c7ccccc7)c6n-4c35)n2)cc1]

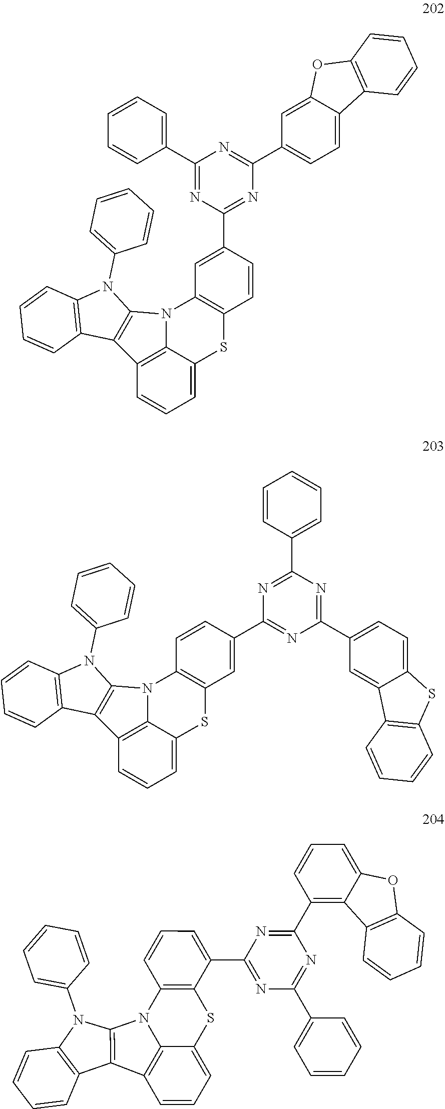 [SMILES: c1ccc(-c2nc(-c3ccc4c(c3)-n3c5c(cccc5c5c6ccccc6n(-c6ccccc6)c53)S4)nc(-c3ccc4c(c3)oc3ccccc34)n2)cc1 ; c1ccc(-c2nc(-c3ccc4c(c3)Sc3cccc5c6c7ccccc7n(-c7ccccc7)c6n-4c35)nc(-c3ccc4sc5ccccc5c4c3)n2)cc1 ; c1ccc(-c2nc(-c3cccc4c3Sc3cccc5c6c7ccccc7n(-c7ccccc7)c6n-4c35)nc(-c3cccc4oc5ccccc5c34)n2)cc1]

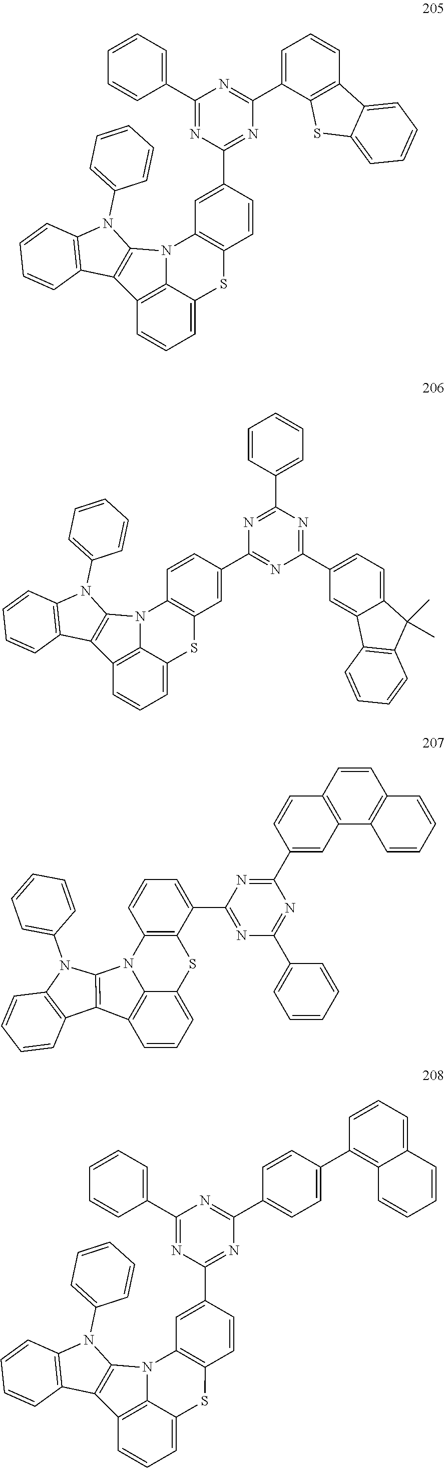 [SMILES: c1ccc(-c2nc(-c3ccc4c(c3)-n3c5c(cccc5c5c6ccccc6n(-c6ccccc6)c53)S4)nc(-c3cccc4c3sc3ccccc34)n2)cc1 ; CC1(C)c2ccccc2-c2cc(-c3nc(-c4ccccc4)nc(-c4ccc5c(c4)Sc4cccc6c7c8ccccc8n(-c8ccccc8)c7n-5c46)n3)ccc21 ; c1ccc(-c2nc(-c3ccc4ccc5ccccc5c4c3)nc(-c3cccc4c3Sc3cccc5c6c7ccccc7n(-c7ccccc7)c6n-4c35)n2)cc1 ; c1ccc(-c2nc(-c3ccc(-c4cccc5ccccc45)cc3)nc(-c3ccc4c(c3)-n3c5c(cccc5c5c6ccccc6n(-c6ccccc6)c53)S4)n2)cc1]

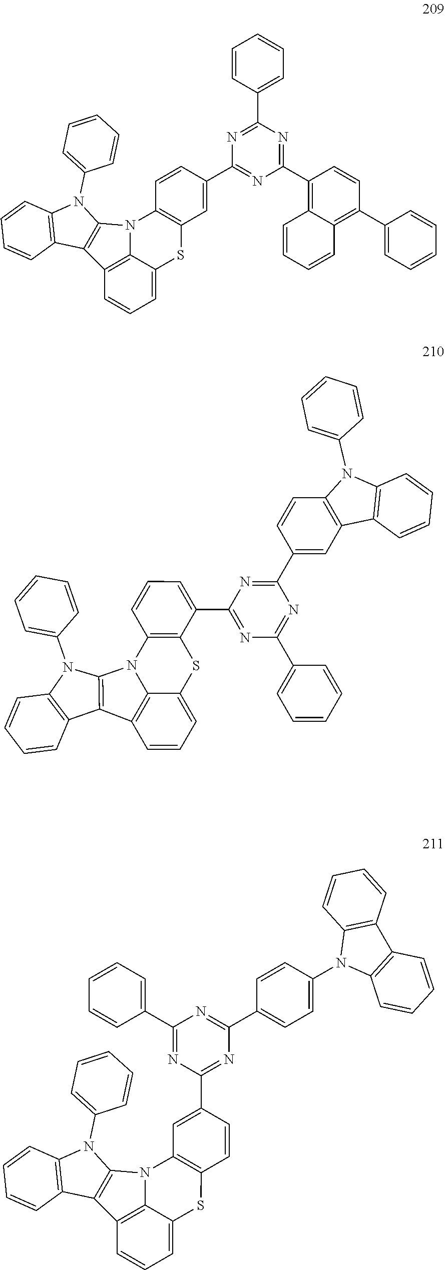 [SMILES: c1ccc(-c2nc(-c3ccc4c(c3)Sc3cccc5c6c7ccccc7n(-c7ccccc7)c6n-4c35)nc(-c3ccc(-c4ccccc4)c4ccccc34)n2)cc1 ; c1ccc(-c2nc(-c3ccc4c(c3)c3ccccc3n4-c3ccccc3)nc(-c3cccc4c3Sc3cccc5c6c7ccccc7n(-c7ccccc7)c6n-4c35)n2)cc1 ; c1ccc(-c2nc(-c3ccc(-n4c5ccccc5c5ccccc54)cc3)nc(-c3ccc4c(c3)-n3c5c(cccc5c5c6ccccc6n(-c6ccccc6)c53)S4)n2)cc1]

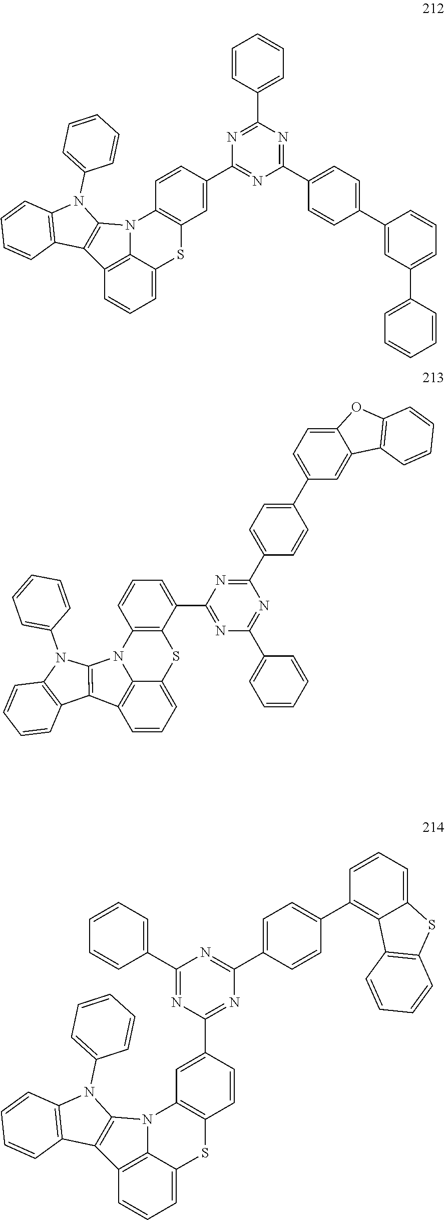 [SMILES: c1ccc(-c2cccc(-c3ccc(-c4nc(-c5ccccc5)nc(-c5ccc6c(c5)Sc5cccc7c8c9ccccc9n(-c9ccccc9)c8n-6c57)n4)cc3)c2)cc1 ; c1ccc(-c2nc(-c3ccc(-c4ccc5oc6ccccc6c5c4)cc3)nc(-c3cccc4c3Sc3cccc5c6c7ccccc7n(-c7ccccc7)c6n-4c35)n2)cc1 ; c1ccc(-c2nc(-c3ccc(-c4cccc5sc6ccccc6c45)cc3)nc(-c3ccc4c(c3)-n3c5c(cccc5c5c6ccccc6n(-c6ccccc6)c53)S4)n2)cc1]

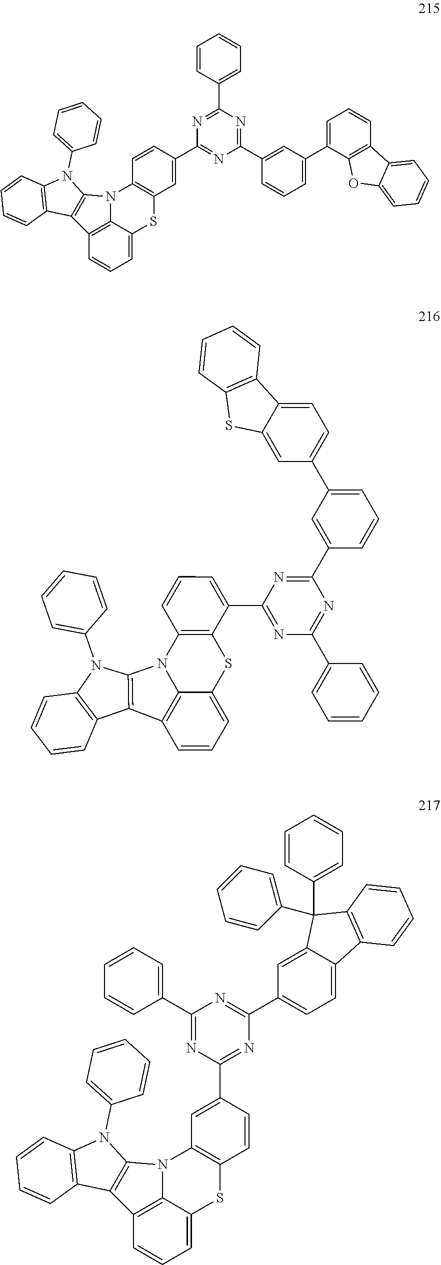 [SMILES: c1ccc(-c2nc(-c3cccc(-c4cccc5c4oc4ccccc45)c3)nc(-c3ccc4c(c3)Sc3cccc5c6c7ccccc7n(-c7ccccc7)c6n-4c35)n2)cc1 ; c1ccc(-c2nc(-c3cccc(-c4ccc5c(c4)sc4ccccc45)c3)nc(-c3cccc4c3Sc3cccc5c6c7ccccc7n(-c7ccccc7)c6n-4c35)n2)cc1 ; c1ccc(-c2nc(-c3ccc4c(c3)-n3c5c(cccc5c5c6ccccc6n(-c6ccccc6)c53)S4)nc(-c3ccc4c(c3)C(c3ccccc3)(c3ccccc3)c3ccccc3-4)n2)cc1]

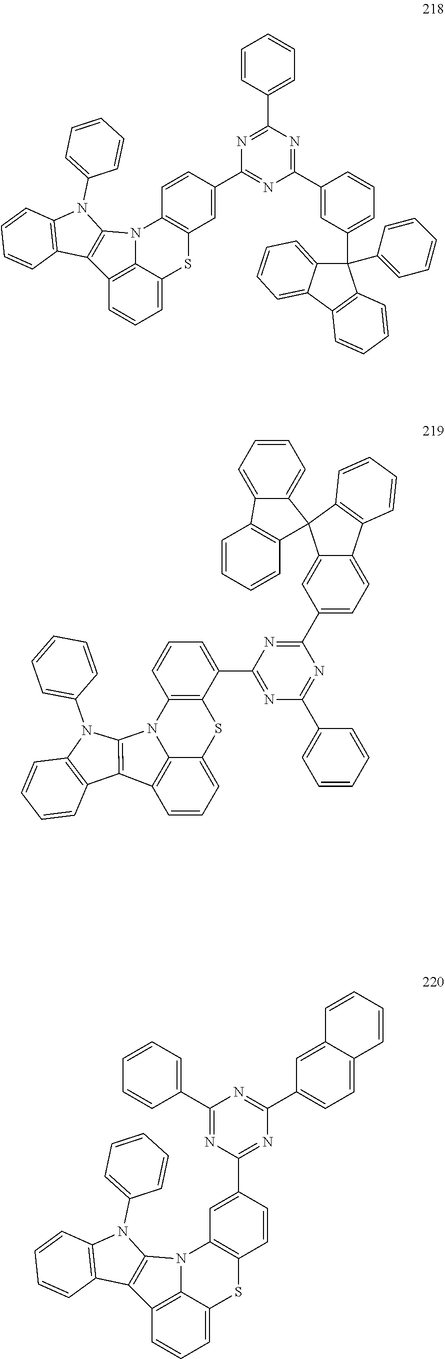 [SMILES: c1ccc(-c2nc(-c3cccc(C4(c5ccccc5)c5ccccc5-c5ccccc54)c3)nc(-c3ccc4c(c3)Sc3cccc5c6c7ccccc7n(-c7ccccc7)c6n-4c35)n2)cc1 ; c1ccc(-c2nc(-c3ccc4c(c3)C3(c5ccccc5-c5ccccc53)c3ccccc3-4)nc(-c3cccc4c3Sc3cccc5c6c7ccccc7n(-c7ccccc7)c6n-4c35)n2)cc1 ; c1ccc(-c2nc(-c3ccc4c(c3)-n3c5c(cccc5c5c6ccccc6n(-c6ccccc6)c53)S4)nc(-c3ccc4ccccc4c3)n2)cc1]

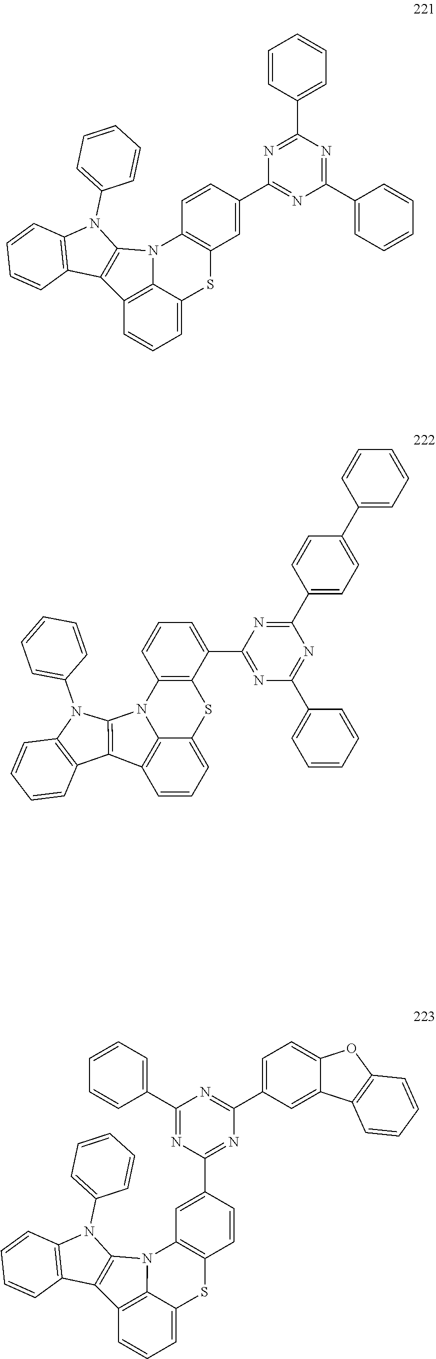 [SMILES: c1ccc(-c2nc(-c3ccccc3)nc(-c3ccc4c(c3)Sc3cccc5c6c7ccccc7n(-c7ccccc7)c6n-4c35)n2)cc1 ; c1ccc(-c2ccc(-c3nc(-c4ccccc4)nc(-c4cccc5c4Sc4cccc6c7c8ccccc8n(-c8ccccc8)c7n-5c46)n3)cc2)cc1 ; c1ccc(-c2nc(-c3ccc4c(c3)-n3c5c(cccc5c5c6ccccc6n(-c6ccccc6)c53)S4)nc(-c3ccc4oc5ccccc5c4c3)n2)cc1]

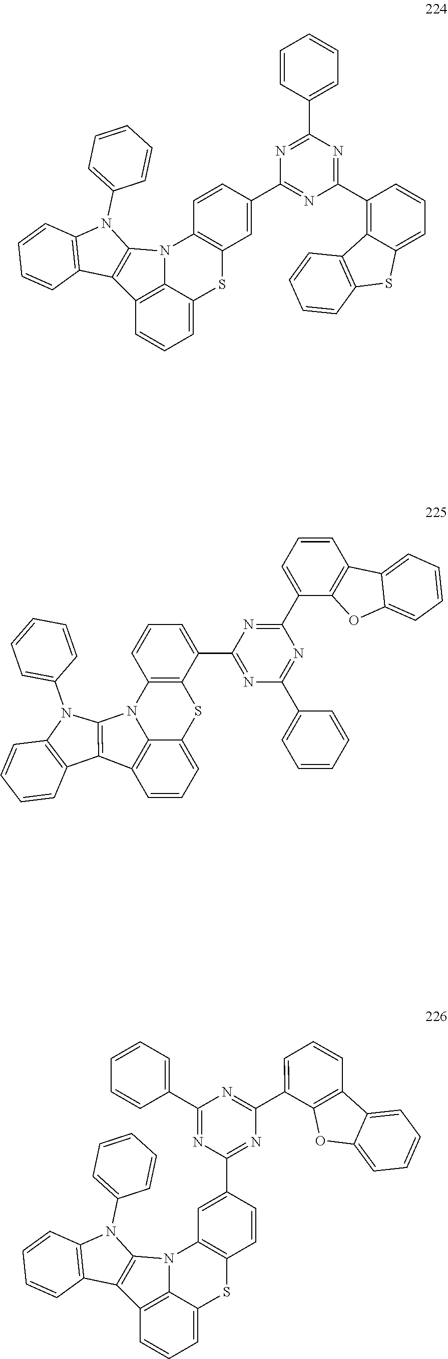 [SMILES: c1ccc(-c2nc(-c3ccc4c(c3)Sc3cccc5c6c7ccccc7n(-c7ccccc7)c6n-4c35)nc(-c3cccc4sc5ccccc5c34)n2)cc1 ; c1ccc(-c2nc(-c3cccc4c3Sc3cccc5c6c7ccccc7n(-c7ccccc7)c6n-4c35)nc(-c3cccc4c3oc3ccccc34)n2)cc1 ; c1ccc(-c2nc(-c3ccc4c(c3)-n3c5c(cccc5c5c6ccccc6n(-c6ccccc6)c53)S4)nc(-c3cccc4c3oc3ccccc34)n2)cc1]

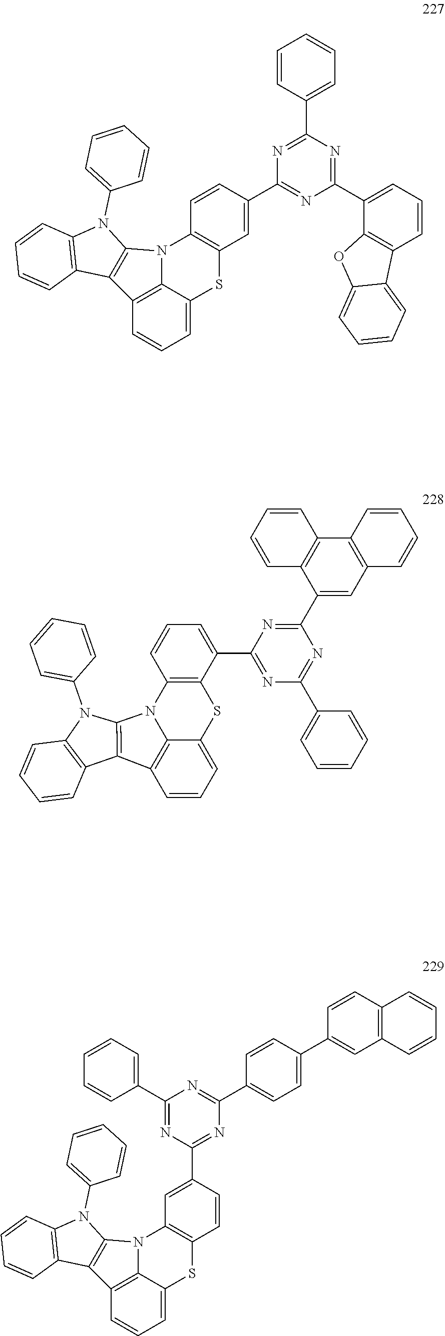 [SMILES: c1ccc(-c2nc(-c3ccc4c(c3)Sc3cccc5c6c7ccccc7n(-c7ccccc7)c6n-4c35)nc(-c3cccc4c3oc3ccccc34)n2)cc1 ; c1ccc(-c2nc(-c3cccc4c3Sc3cccc5c6c7ccccc7n(-c7ccccc7)c6n-4c35)nc(-c3cc4ccccc4c4ccccc34)n2)cc1 ; c1ccc(-c2nc(-c3ccc(-c4ccc5ccccc5c4)cc3)nc(-c3ccc4c(c3)-n3c5c(cccc5c5c6ccccc6n(-c6ccccc6)c53)S4)n2)cc1]

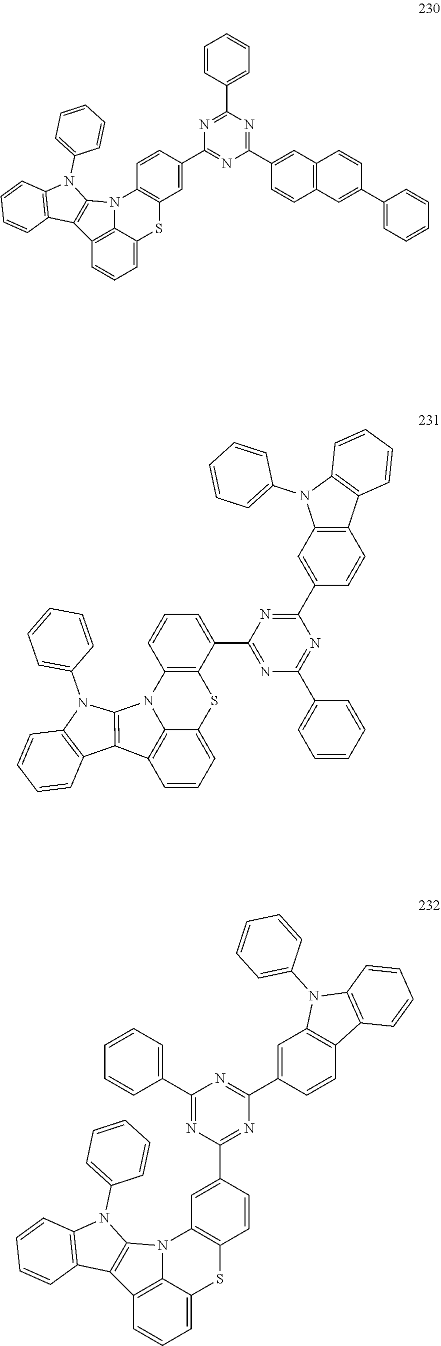 [SMILES: c1ccc(-c2ccc3cc(-c4nc(-c5ccccc5)nc(-c5ccc6c(c5)Sc5cccc7c8c9ccccc9n(-c9ccccc9)c8n-6c57)n4)ccc3c2)cc1 ; c1ccc(-c2nc(-c3ccc4c5ccccc5n(-c5ccccc5)c4c3)nc(-c3cccc4c3Sc3cccc5c6c7ccccc7n(-c7ccccc7)c6n-4c35)n2)cc1 ; c1ccc(-c2nc(-c3ccc4c(c3)-n3c5c(cccc5c5c6ccccc6n(-c6ccccc6)c53)S4)nc(-c3ccc4c5ccccc5n(-c5ccccc5)c4c3)n2)cc1]

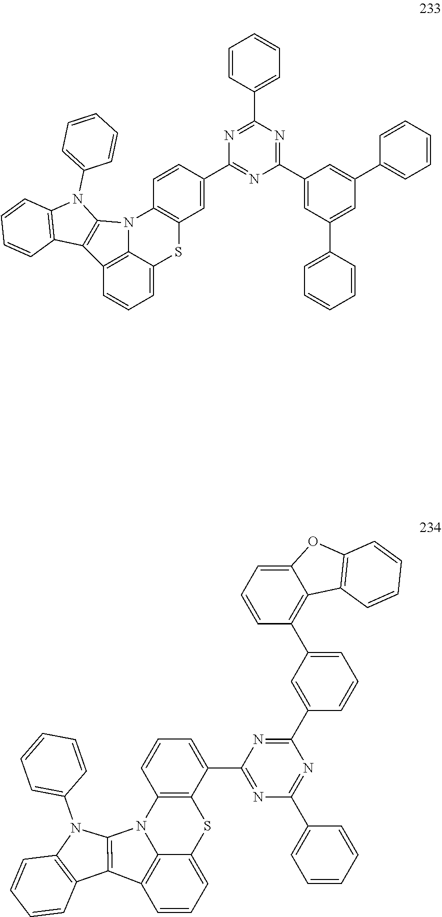 [SMILES: c1ccc(-c2cc(-c3ccccc3)cc(-c3nc(-c4ccccc4)nc(-c4ccc5c(c4)Sc4cccc6c7c8ccccc8n(-c8ccccc8)c7n-5c46)n3)c2)cc1 ; c1ccc(-c2nc(-c3cccc(-c4cccc5oc6ccccc6c45)c3)nc(-c3cccc4c3Sc3cccc5c6c7ccccc7n(-c7ccccc7)c6n-4c35)n2)cc1]

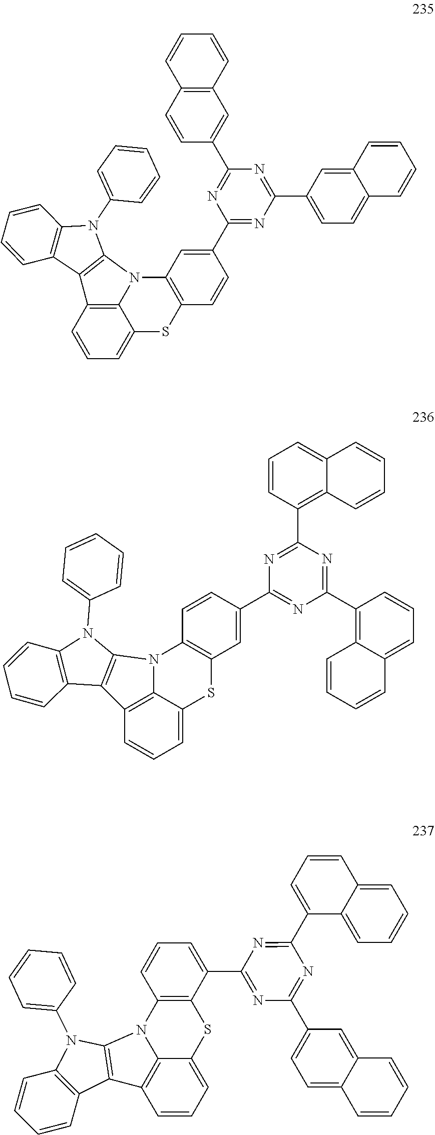 [SMILES: c1ccc(-n2c3ccccc3c3c4cccc5c4n(c32)-c2cc(-c3nc(-c4ccc6ccccc6c4)nc(-c4ccc6ccccc6c4)n3)ccc2S5)cc1 ; c1ccc(-n2c3ccccc3c3c4cccc5c4n(c32)-c2ccc(-c3nc(-c4cccc6ccccc46)nc(-c4cccc6ccccc46)n3)cc2S5)cc1 ; c1ccc(-n2c3ccccc3c3c4cccc5c4n(c32)-c2cccc(-c3nc(-c4ccc6ccccc6c4)nc(-c4cccc6ccccc46)n3)c2S5)cc1]

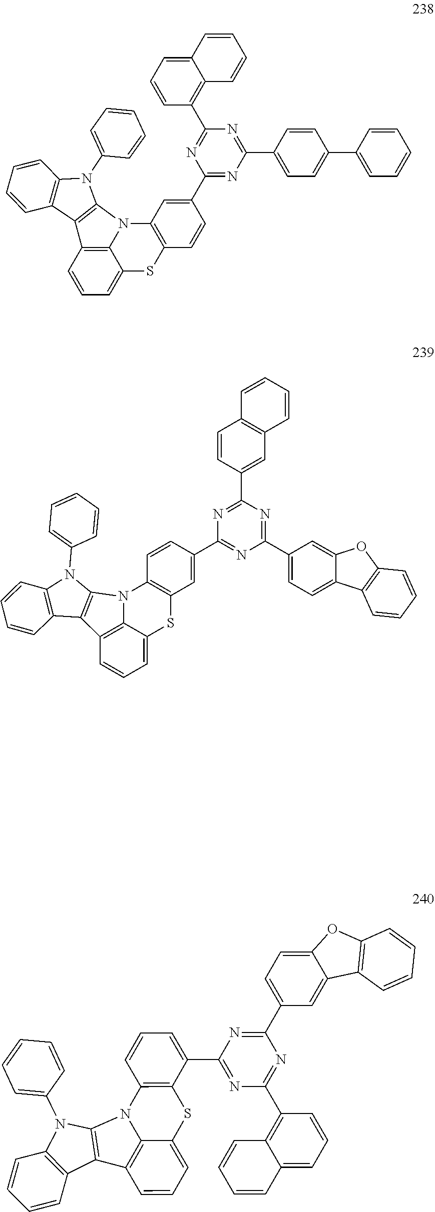 [SMILES: c1ccc(-c2ccc(-c3nc(-c4ccc5c(c4)-n4c6c(cccc6c6c7ccccc7n(-c7ccccc7)c64)S5)nc(-c4cccc5ccccc45)n3)cc2)cc1 ; c1ccc(-n2c3ccccc3c3c4cccc5c4n(c32)-c2ccc(-c3nc(-c4ccc6ccccc6c4)nc(-c4ccc6c(c4)oc4ccccc46)n3)cc2S5)cc1 ; c1ccc(-n2c3ccccc3c3c4cccc5c4n(c32)-c2cccc(-c3nc(-c4ccc6oc7ccccc7c6c4)nc(-c4cccc6ccccc46)n3)c2S5)cc1]

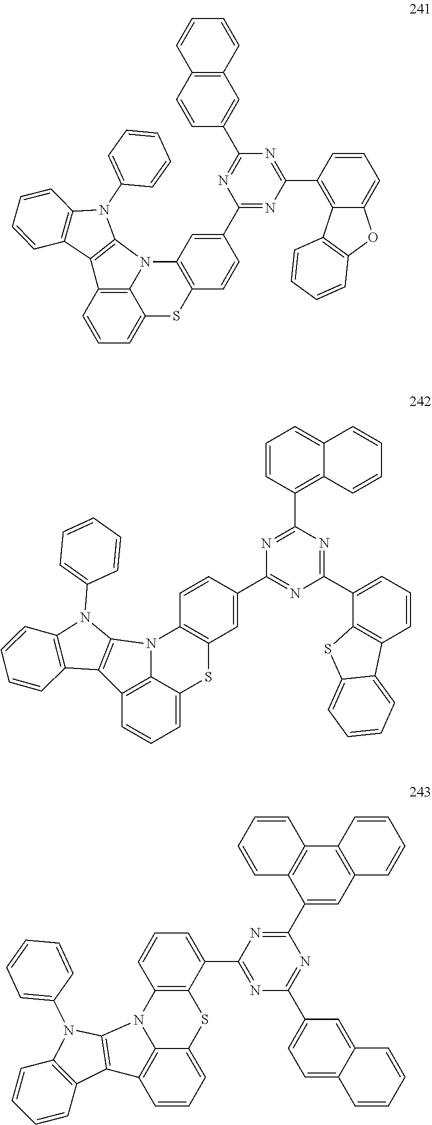 [SMILES: c1ccc(-n2c3ccccc3c3c4cccc5c4n(c32)-c2cc(-c3nc(-c4ccc6ccccc6c4)nc(-c4cccc6oc7ccccc7c46)n3)ccc2S5)cc1 ; c1ccc(-n2c3ccccc3c3c4cccc5c4n(c32)-c2ccc(-c3nc(-c4cccc6ccccc46)nc(-c4cccc6c4sc4ccccc46)n3)cc2S5)cc1 ; c1ccc(-n2c3ccccc3c3c4cccc5c4n(c32)-c2cccc(-c3nc(-c4ccc6ccccc6c4)nc(-c4cc6ccccc6c6ccccc46)n3)c2S5)cc1]

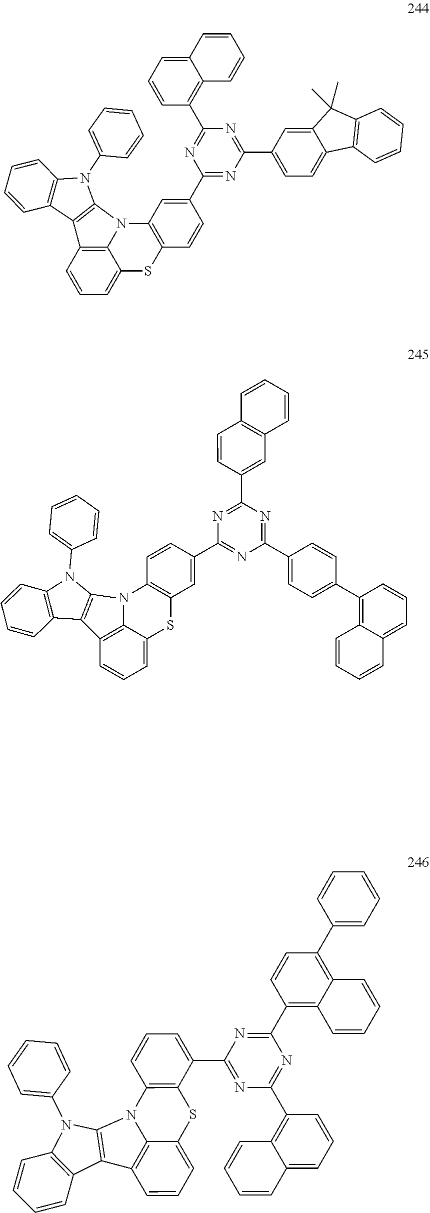 [SMILES: CC1(C)c2ccccc2-c2ccc(-c3nc(-c4ccc5c(c4)-n4c6c(cccc6c6c7ccccc7n(-c7ccccc7)c64)S5)nc(-c4cccc5ccccc45)n3)cc21 ; c1ccc(-n2c3ccccc3c3c4cccc5c4n(c32)-c2ccc(-c3nc(-c4ccc(-c6cccc7ccccc67)cc4)nc(-c4ccc6ccccc6c4)n3)cc2S5)cc1 ; c1ccc(-c2ccc(-c3nc(-c4cccc5c4Sc4cccc6c7c8ccccc8n(-c8ccccc8)c7n-5c46)nc(-c4cccc5ccccc45)n3)c3ccccc23)cc1]

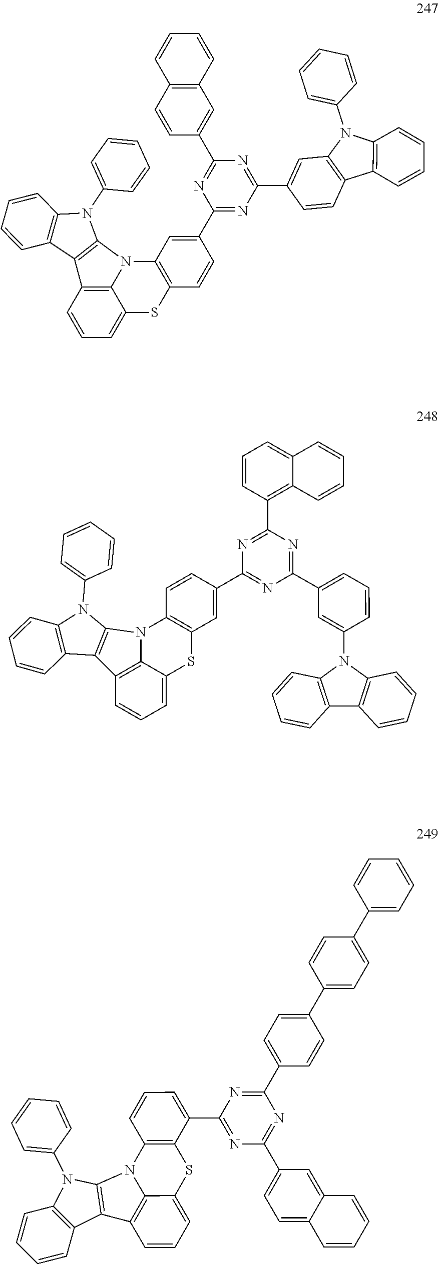 [SMILES: c1ccc(-n2c3ccccc3c3ccc(-c4nc(-c5ccc6c(c5)-n5c7c(cccc7c7c8ccccc8n(-c8ccccc8)c75)S6)nc(-c5ccc6ccccc6c5)n4)cc32)cc1 ; c1ccc(-n2c3ccccc3c3c4cccc5c4n(c32)-c2ccc(-c3nc(-c4cccc(-n6c7ccccc7c7ccccc76)c4)nc(-c4cccc6ccccc46)n3)cc2S5)cc1 ; c1ccc(-c2ccc(-c3ccc(-c4nc(-c5ccc6ccccc6c5)nc(-c5cccc6c5Sc5cccc7c8c9ccccc9n(-c9ccccc9)c8n-6c57)n4)cc3)cc2)cc1]

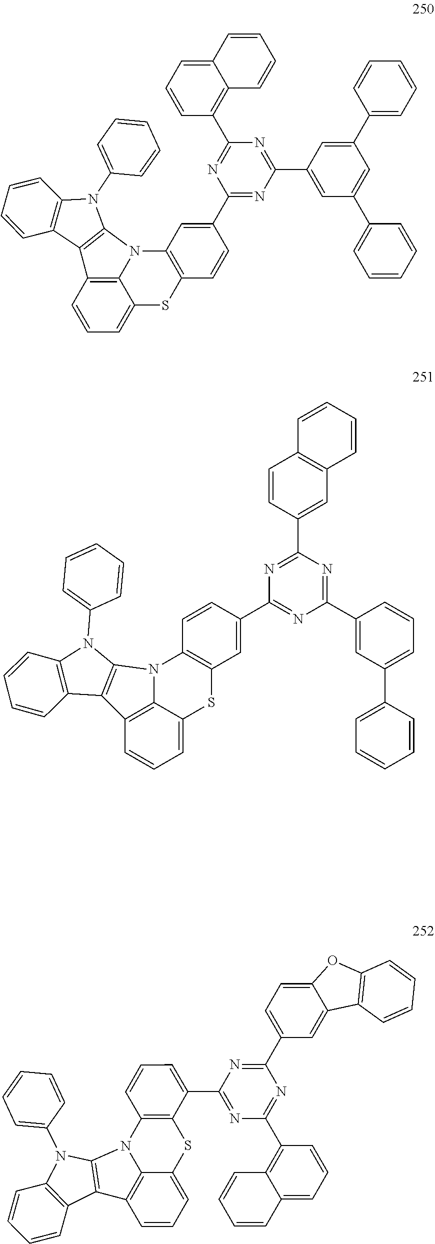 [SMILES: c1ccc(-c2cc(-c3ccccc3)cc(-c3nc(-c4ccc5c(c4)-n4c6c(cccc6c6c7ccccc7n(-c7ccccc7)c64)S5)nc(-c4cccc5ccccc45)n3)c2)cc1 ; c1ccc(-c2cccc(-c3nc(-c4ccc5c(c4)Sc4cccc6c7c8ccccc8n(-c8ccccc8)c7n-5c46)nc(-c4ccc5ccccc5c4)n3)c2)cc1 ; c1ccc(-n2c3ccccc3c3c4cccc5c4n(c32)-c2cccc(-c3nc(-c4ccc6oc7ccccc7c6c4)nc(-c4cccc6ccccc46)n3)c2S5)cc1]

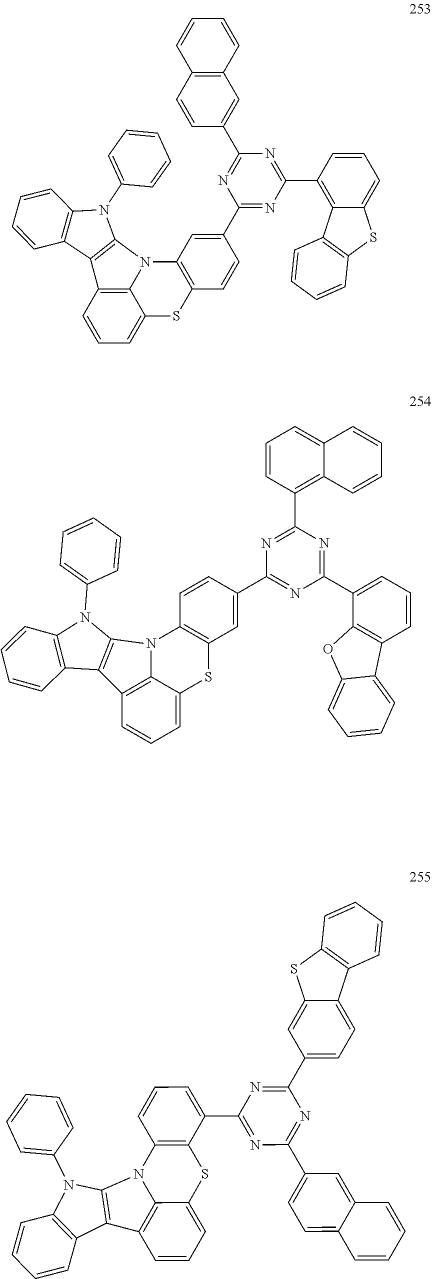 [SMILES: c1ccc(-n2c3ccccc3c3c4cccc5c4n(c32)-c2cc(-c3nc(-c4ccc6ccccc6c4)nc(-c4cccc6sc7ccccc7c46)n3)ccc2S5)cc1 ; c1ccc(-n2c3ccccc3c3c4cccc5c4n(c32)-c2ccc(-c3nc(-c4cccc6ccccc46)nc(-c4cccc6c4oc4ccccc46)n3)cc2S5)cc1 ; c1ccc(-n2c3ccccc3c3c4cccc5c4n(c32)-c2cccc(-c3nc(-c4ccc6ccccc6c4)nc(-c4ccc6c(c4)sc4ccccc46)n3)c2S5)cc1]

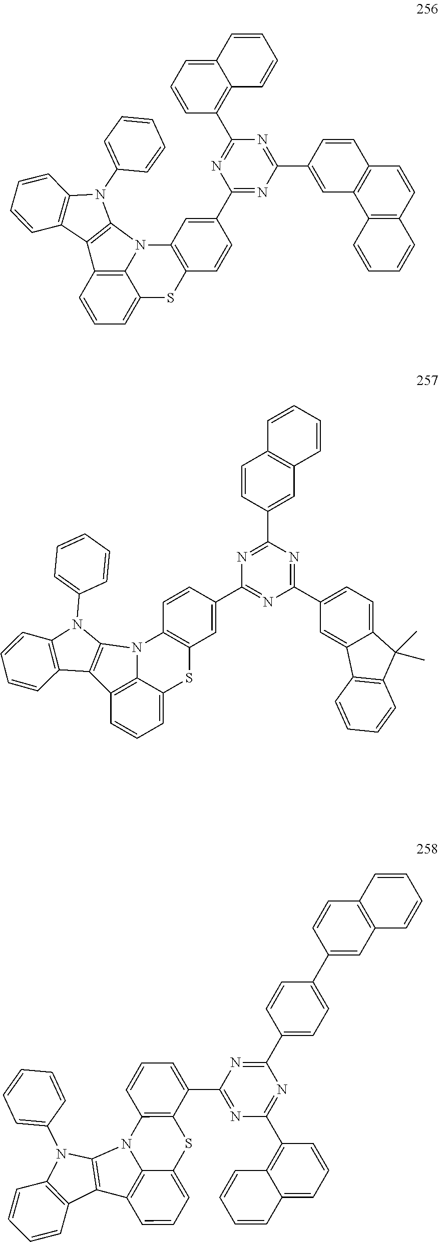 [SMILES: c1ccc(-n2c3ccccc3c3c4cccc5c4n(c32)-c2cc(-c3nc(-c4ccc6ccc7ccccc7c6c4)nc(-c4cccc6ccccc46)n3)ccc2S5)cc1 ; CC1(C)c2ccccc2-c2cc(-c3nc(-c4ccc5c(c4)Sc4cccc6c7c8ccccc8n(-c8ccccc8)c7n-5c46)nc(-c4ccc5ccccc5c4)n3)ccc21 ; c1ccc(-n2c3ccccc3c3c4cccc5c4n(c32)-c2cccc(-c3nc(-c4ccc(-c6ccc7ccccc7c6)cc4)nc(-c4cccc6ccccc46)n3)c2S5)cc1]

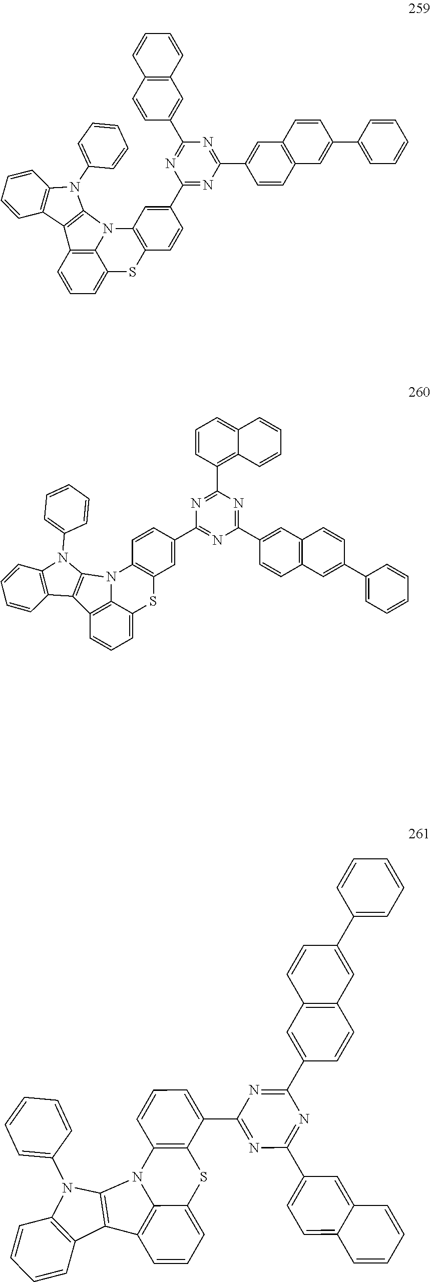 [SMILES: c1ccc(-c2ccc3cc(-c4nc(-c5ccc6c(c5)-n5c7c(cccc7c7c8ccccc8n(-c8ccccc8)c75)S6)nc(-c5ccc6ccccc6c5)n4)ccc3c2)cc1 ; c1ccc(-c2ccc3cc(-c4nc(-c5ccc6c(c5)Sc5cccc7c8c9ccccc9n(-c9ccccc9)c8n-6c57)nc(-c5cccc6ccccc56)n4)ccc3c2)cc1 ; c1ccc(-c2ccc3cc(-c4nc(-c5ccc6ccccc6c5)nc(-c5cccc6c5Sc5cccc7c8c9ccccc9n(-c9ccccc9)c8n-6c57)n4)ccc3c2)cc1]

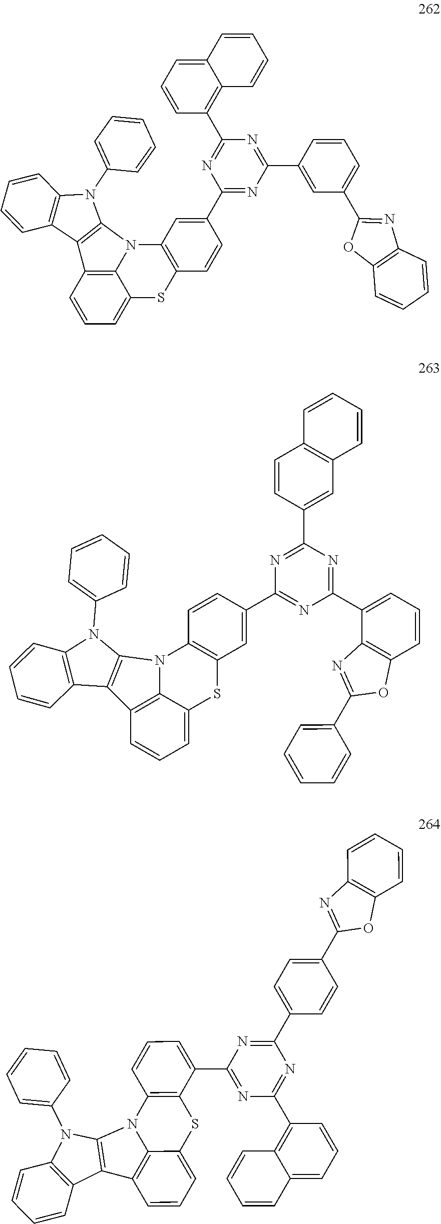 [SMILES: c1ccc(-n2c3ccccc3c3c4cccc5c4n(c32)-c2cc(-c3nc(-c4cccc(-c6nc7ccccc7o6)c4)nc(-c4cccc6ccccc46)n3)ccc2S5)cc1 ; c1ccc(-c2nc3c(-c4nc(-c5ccc6c(c5)Sc5cccc7c8c9ccccc9n(-c9ccccc9)c8n-6c57)nc(-c5ccc6ccccc6c5)n4)cccc3o2)cc1 ; c1ccc(-n2c3ccccc3c3c4cccc5c4n(c32)-c2cccc(-c3nc(-c4ccc(-c6nc7ccccc7o6)cc4)nc(-c4cccc6ccccc46)n3)c2S5)cc1]

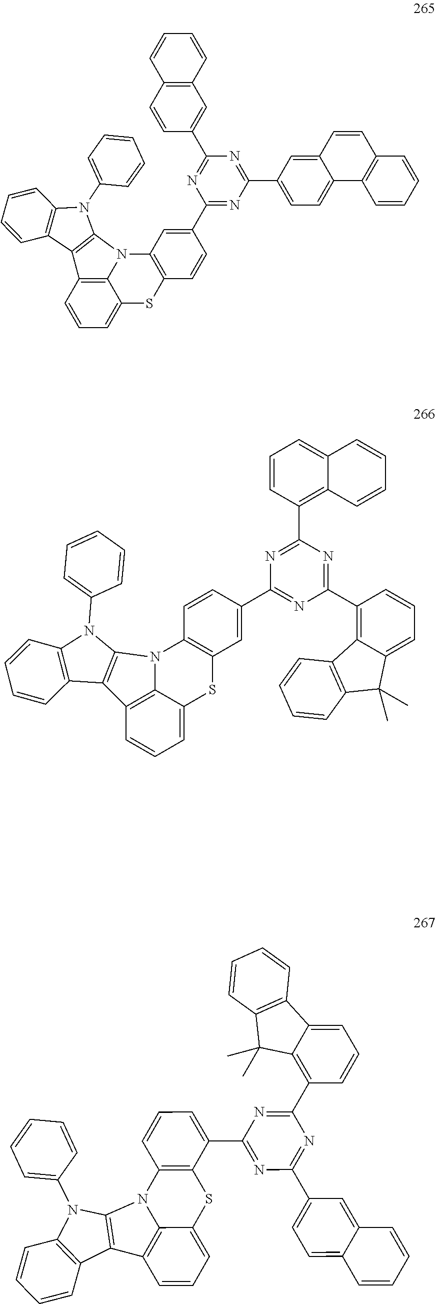 [SMILES: c1ccc(-n2c3ccccc3c3c4cccc5c4n(c32)-c2cc(-c3nc(-c4ccc6ccccc6c4)nc(-c4ccc6c(ccc7ccccc76)c4)n3)ccc2S5)cc1 ; CC1(C)c2ccccc2-c2c(-c3nc(-c4ccc5c(c4)Sc4cccc6c7c8ccccc8n(-c8ccccc8)c7n-5c46)nc(-c4cccc5ccccc45)n3)cccc21 ; CC1(C)c2ccccc2-c2cccc(-c3nc(-c4ccc5ccccc5c4)nc(-c4cccc5c4Sc4cccc6c7c8ccccc8n(-c8ccccc8)c7n-5c46)n3)c21]

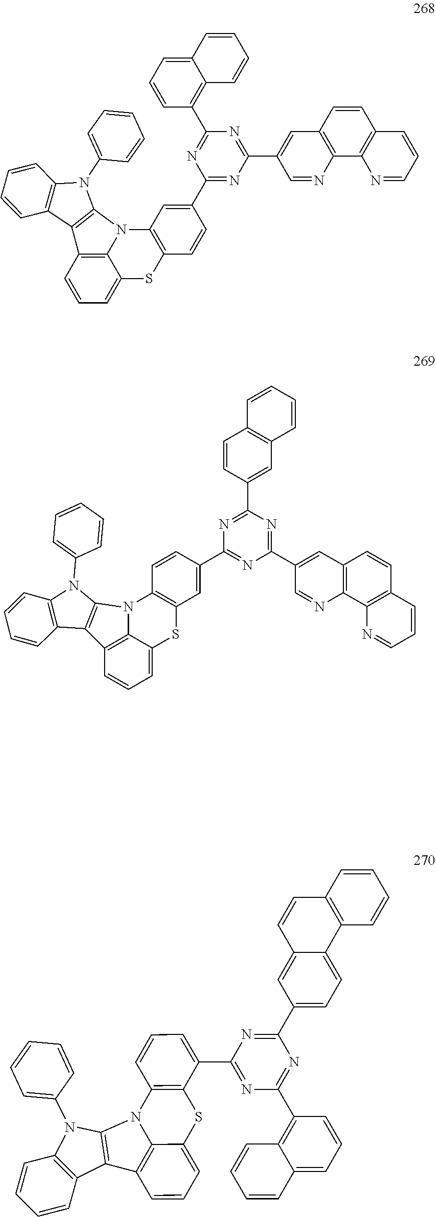 [SMILES: c1ccc(-n2c3ccccc3c3c4cccc5c4n(c32)-c2cc(-c3nc(-c4cnc6c(ccc7cccnc76)c4)nc(-c4cccc6ccccc46)n3)ccc2S5)cc1 ; c1ccc(-n2c3ccccc3c3c4cccc5c4n(c32)-c2ccc(-c3nc(-c4ccc6ccccc6c4)nc(-c4cnc6c(ccc7cccnc76)c4)n3)cc2S5)cc1 ; c1ccc(-n2c3ccccc3c3c4cccc5c4n(c32)-c2cccc(-c3nc(-c4ccc6c(ccc7ccccc76)c4)nc(-c4cccc6ccccc46)n3)c2S5)cc1]

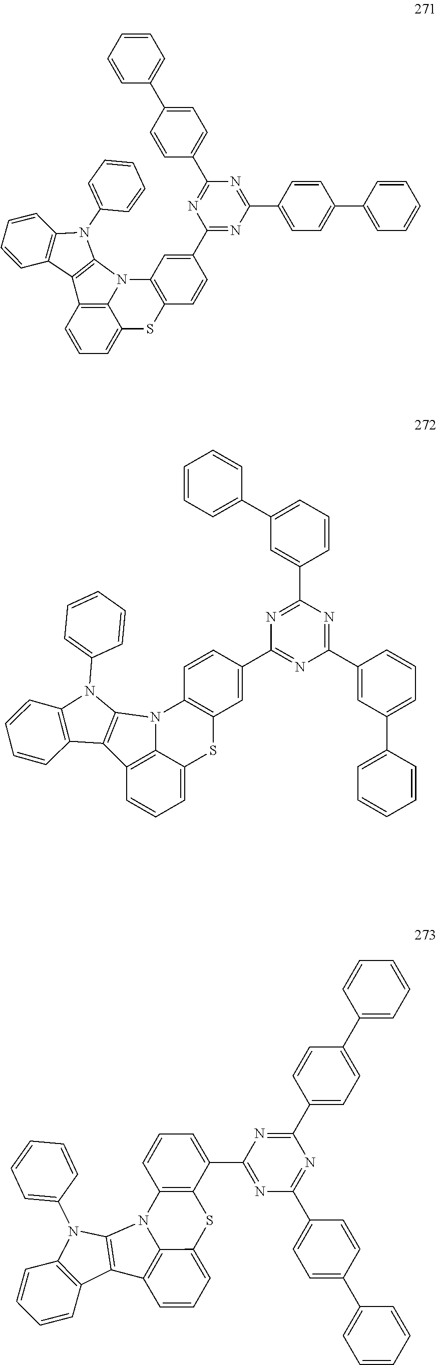 [SMILES: c1ccc(-c2ccc(-c3nc(-c4ccc(-c5ccccc5)cc4)nc(-c4ccc5c(c4)-n4c6c(cccc6c6c7ccccc7n(-c7ccccc7)c64)S5)n3)cc2)cc1 ; c1ccc(-c2cccc(-c3nc(-c4cccc(-c5ccccc5)c4)nc(-c4ccc5c(c4)Sc4cccc6c7c8ccccc8n(-c8ccccc8)c7n-5c46)n3)c2)cc1 ; c1ccc(-c2ccc(-c3nc(-c4ccc(-c5ccccc5)cc4)nc(-c4cccc5c4Sc4cccc6c7c8ccccc8n(-c8ccccc8)c7n-5c46)n3)cc2)cc1]

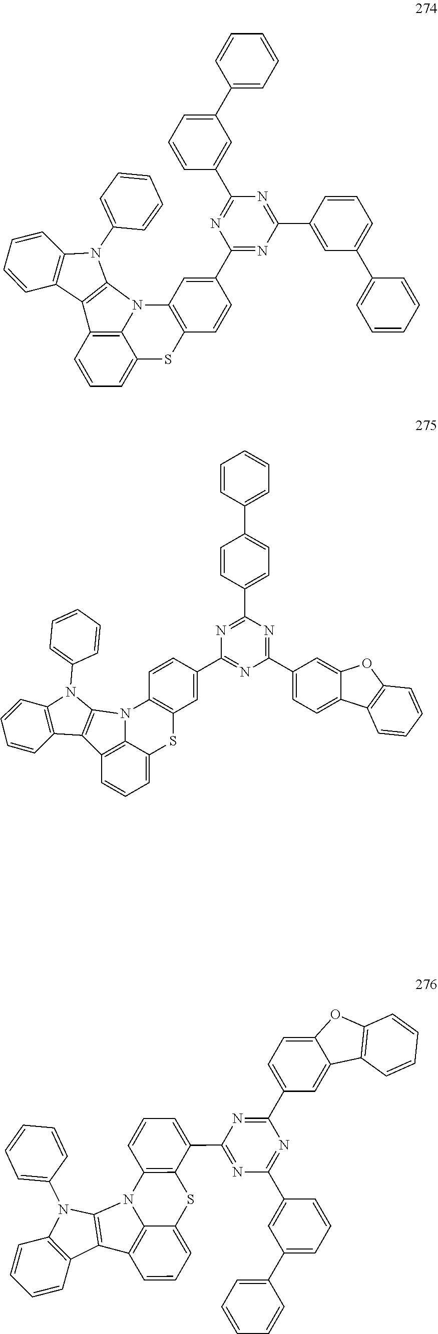 [SMILES: c1ccc(-c2cccc(-c3nc(-c4cccc(-c5ccccc5)c4)nc(-c4ccc5c(c4)-n4c6c(cccc6c6c7ccccc7n(-c7ccccc7)c64)S5)n3)c2)cc1 ; c1ccc(-c2ccc(-c3nc(-c4ccc5c(c4)Sc4cccc6c7c8ccccc8n(-c8ccccc8)c7n-5c46)nc(-c4ccc5c(c4)oc4ccccc45)n3)cc2)cc1 ; c1ccc(-c2cccc(-c3nc(-c4ccc5oc6ccccc6c5c4)nc(-c4cccc5c4Sc4cccc6c7c8ccccc8n(-c8ccccc8)c7n-5c46)n3)c2)cc1]

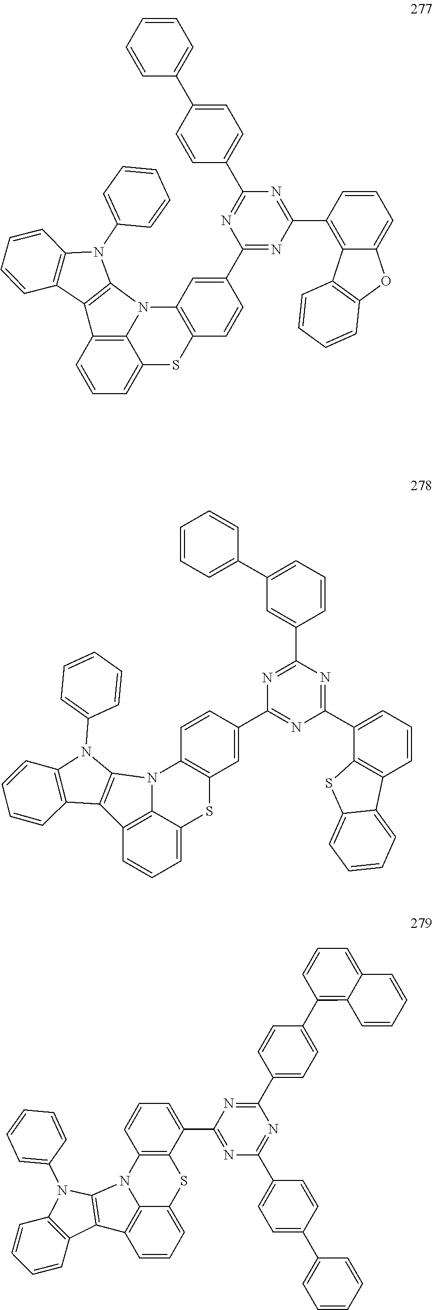 [SMILES: c1ccc(-c2ccc(-c3nc(-c4ccc5c(c4)-n4c6c(cccc6c6c7ccccc7n(-c7ccccc7)c64)S5)nc(-c4cccc5oc6ccccc6c45)n3)cc2)cc1 ; c1ccc(-c2cccc(-c3nc(-c4ccc5c(c4)Sc4cccc6c7c8ccccc8n(-c8ccccc8)c7n-5c46)nc(-c4cccc5c4sc4ccccc45)n3)c2)cc1 ; c1ccc(-c2ccc(-c3nc(-c4ccc(-c5cccc6ccccc56)cc4)nc(-c4cccc5c4Sc4cccc6c7c8ccccc8n(-c8ccccc8)c7n-5c46)n3)cc2)cc1]

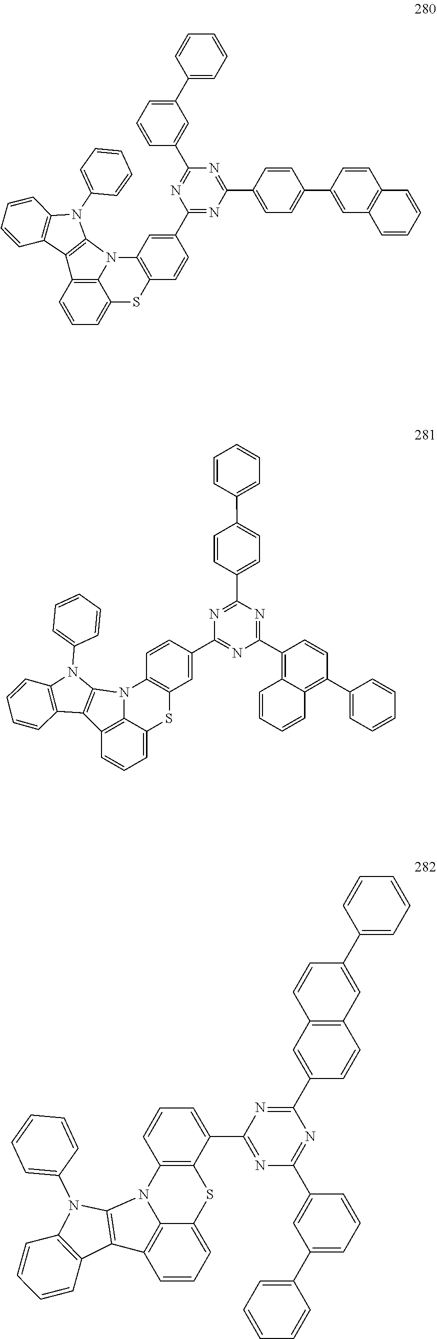 [SMILES: c1ccc(-c2cccc(-c3nc(-c4ccc(-c5ccc6ccccc6c5)cc4)nc(-c4ccc5c(c4)-n4c6c(cccc6c6c7ccccc7n(-c7ccccc7)c64)S5)n3)c2)cc1 ; c1ccc(-c2ccc(-c3nc(-c4ccc5c(c4)Sc4cccc6c7c8ccccc8n(-c8ccccc8)c7n-5c46)nc(-c4ccc(-c5ccccc5)c5ccccc45)n3)cc2)cc1 ; c1ccc(-c2cccc(-c3nc(-c4ccc5cc(-c6ccccc6)ccc5c4)nc(-c4cccc5c4Sc4cccc6c7c8ccccc8n(-c8ccccc8)c7n-5c46)n3)c2)cc1]

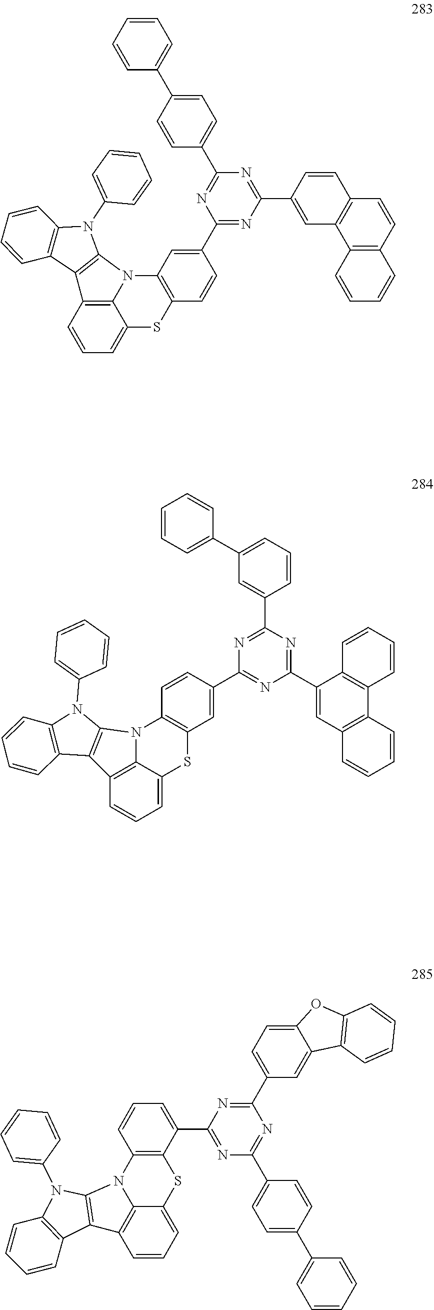 [SMILES: c1ccc(-c2ccc(-c3nc(-c4ccc5c(c4)-n4c6c(cccc6c6c7ccccc7n(-c7ccccc7)c64)S5)nc(-c4ccc5ccc6ccccc6c5c4)n3)cc2)cc1 ; c1ccc(-c2cccc(-c3nc(-c4ccc5c(c4)Sc4cccc6c7c8ccccc8n(-c8ccccc8)c7n-5c46)nc(-c4cc5ccccc5c5ccccc45)n3)c2)cc1 ; c1ccc(-c2ccc(-c3nc(-c4ccc5oc6ccccc6c5c4)nc(-c4cccc5c4Sc4cccc6c7c8ccccc8n(-c8ccccc8)c7n-5c46)n3)cc2)cc1]

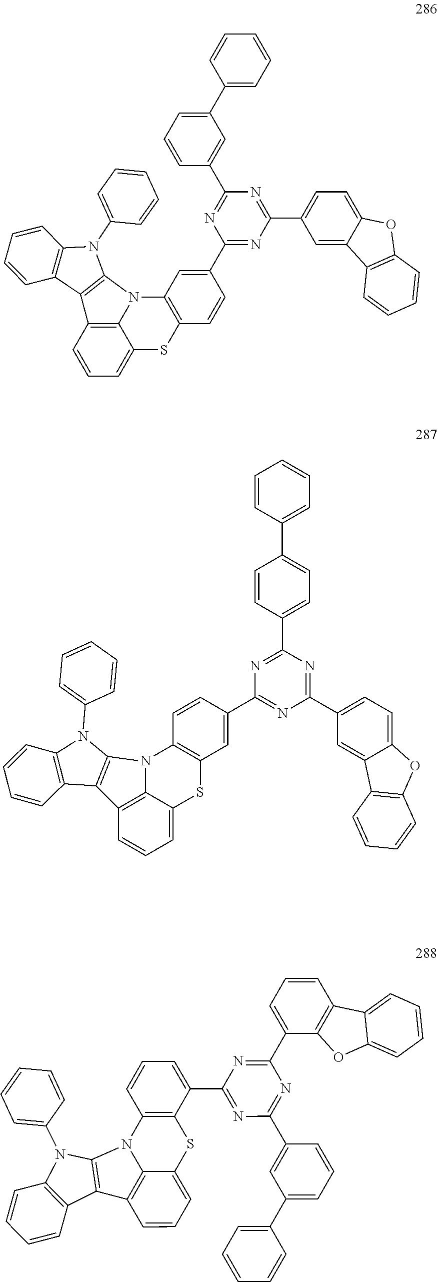 [SMILES: c1ccc(-c2cccc(-c3nc(-c4ccc5c(c4)-n4c6c(cccc6c6c7ccccc7n(-c7ccccc7)c64)S5)nc(-c4ccc5oc6ccccc6c5c4)n3)c2)cc1 ; c1ccc(-c2ccc(-c3nc(-c4ccc5c(c4)Sc4cccc6c7c8ccccc8n(-c8ccccc8)c7n-5c46)nc(-c4ccc5oc6ccccc6c5c4)n3)cc2)cc1 ; c1ccc(-c2cccc(-c3nc(-c4cccc5c4Sc4cccc6c7c8ccccc8n(-c8ccccc8)c7n-5c46)nc(-c4cccc5c4oc4ccccc45)n3)c2)cc1]

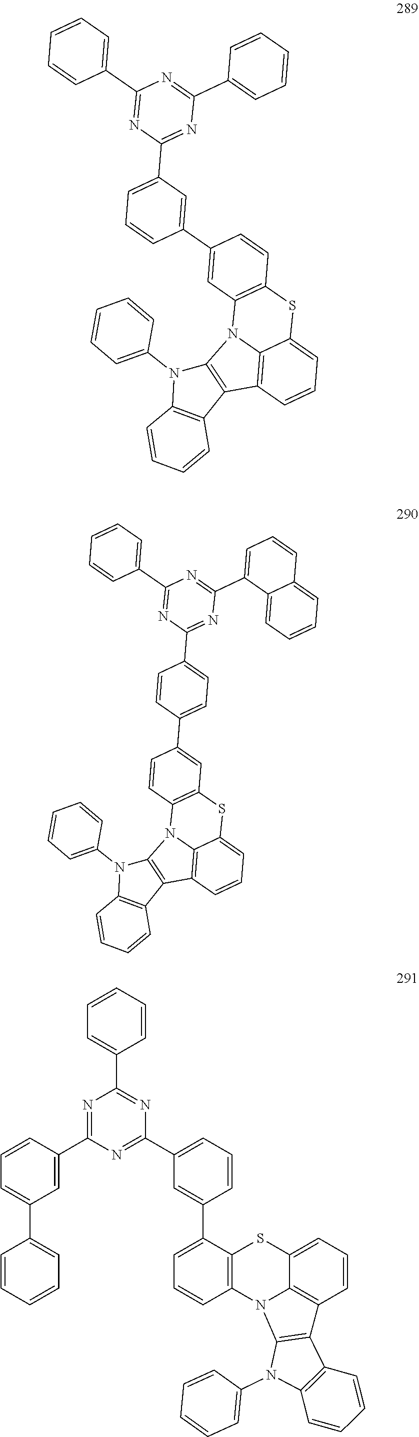

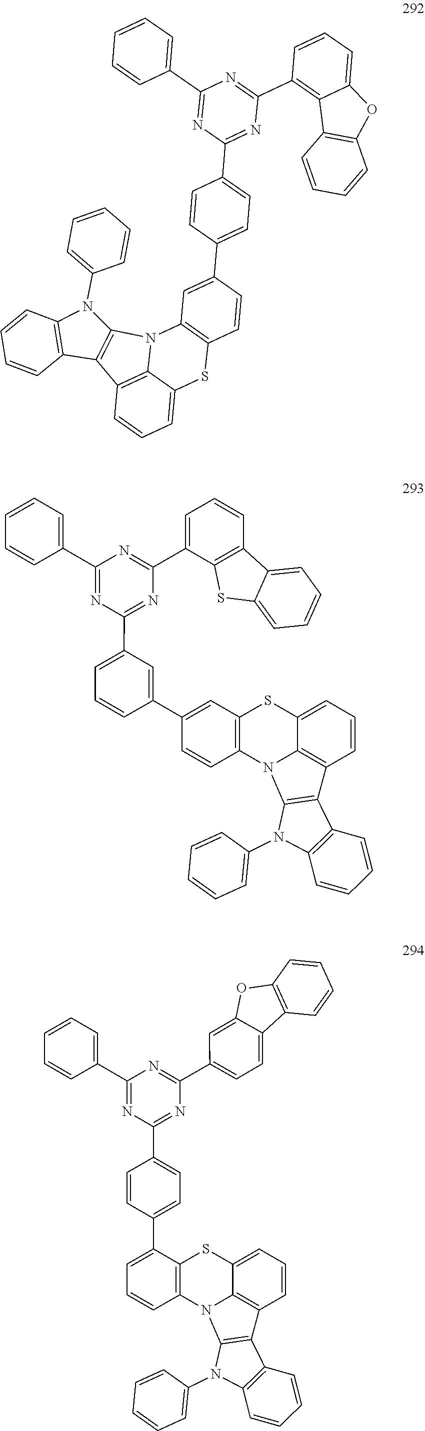 [SMILES: c1ccc(-c2nc(-c3ccc(-c4ccc5c(c4)-n4c6c(cccc6c6c7ccccc7n(-c7ccccc7)c64)S5)cc3)nc(-c3cccc4oc5ccccc5c34)n2)cc1 ; c1ccc(-c2nc(-c3cccc(-c4ccc5c(c4)Sc4cccc6c7c8ccccc8n(-c8ccccc8)c7n-5c46)c3)nc(-c3cccc4c3sc3ccccc34)n2)cc1 ; c1ccc(-c2nc(-c3ccc(-c4cccc5c4Sc4cccc6c7c8ccccc8n(-c8ccccc8)c7n-5c46)cc3)nc(-c3ccc4c(c3)oc3ccccc34)n2)cc1]

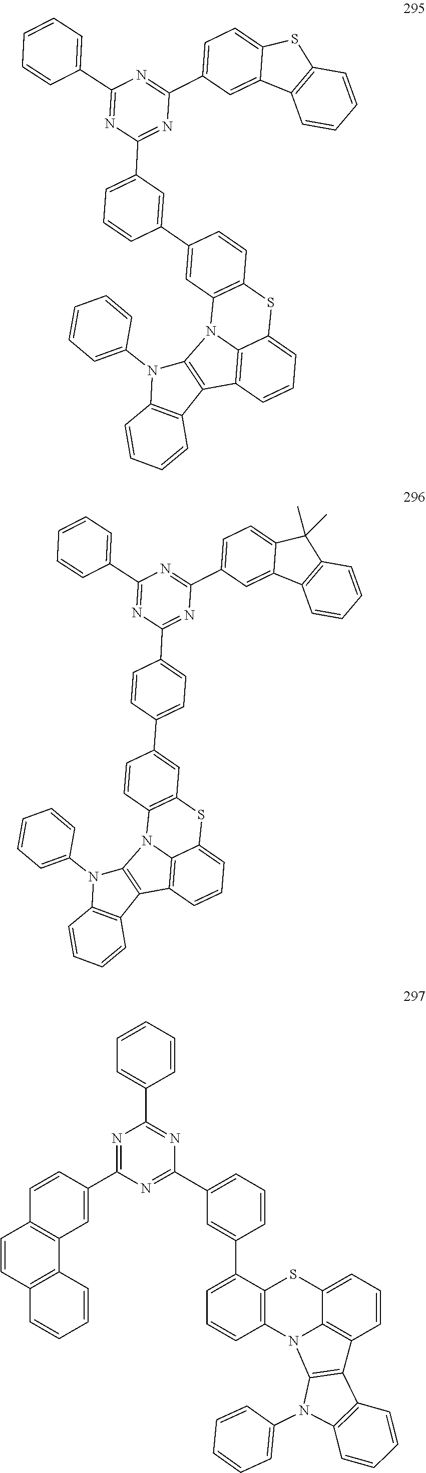 [SMILES: c1ccc(-c2nc(-c3cccc(-c4ccc5c(c4)-n4c6c(cccc6c6c7ccccc7n(-c7ccccc7)c64)S5)c3)nc(-c3ccc4sc5ccccc5c4c3)n2)cc1 ; CC1(C)c2ccccc2-c2cc(-c3nc(-c4ccccc4)nc(-c4ccc(-c5ccc6c(c5)Sc5cccc7c8c9ccccc9n(-c9ccccc9)c8n-6c57)cc4)n3)ccc21 ; c1ccc(-c2nc(-c3cccc(-c4cccc5c4Sc4cccc6c7c8ccccc8n(-c8ccccc8)c7n-5c46)c3)nc(-c3ccc4ccc5ccccc5c4c3)n2)cc1]

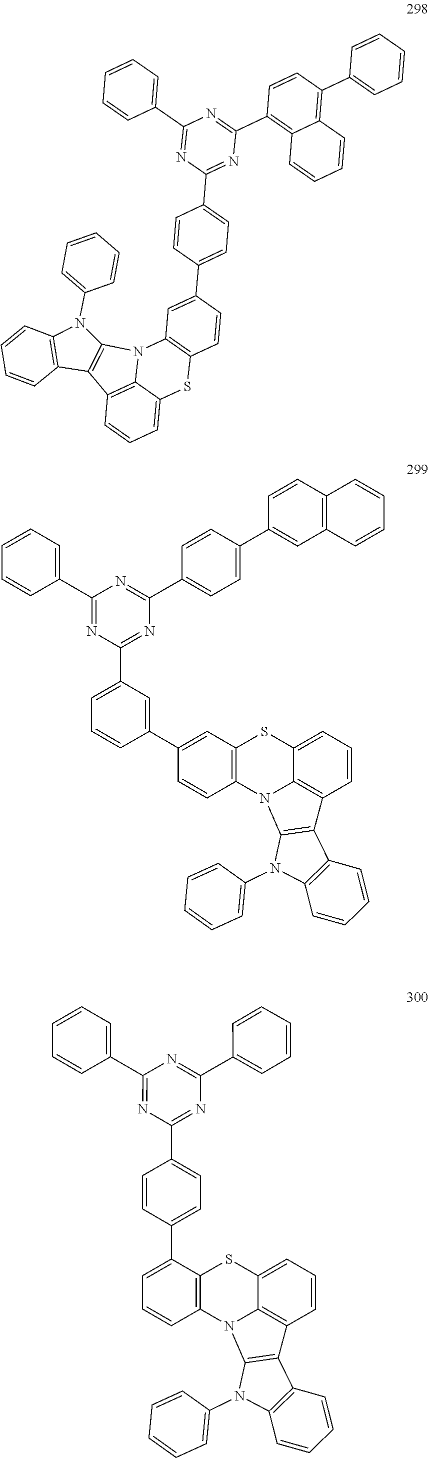 [SMILES: c1ccc(-c2nc(-c3ccc(-c4ccc5c(c4)-n4c6c(cccc6c6c7ccccc7n(-c7ccccc7)c64)S5)cc3)nc(-c3ccc(-c4ccccc4)c4ccccc34)n2)cc1 ; c1ccc(-c2nc(-c3ccc(-c4ccc5ccccc5c4)cc3)nc(-c3cccc(-c4ccc5c(c4)Sc4cccc6c7c8ccccc8n(-c8ccccc8)c7n-5c46)c3)n2)cc1 ; c1ccc(-c2nc(-c3ccccc3)nc(-c3ccc(-c4cccc5c4Sc4cccc6c7c8ccccc8n(-c8ccccc8)c7n-5c46)cc3)n2)cc1]

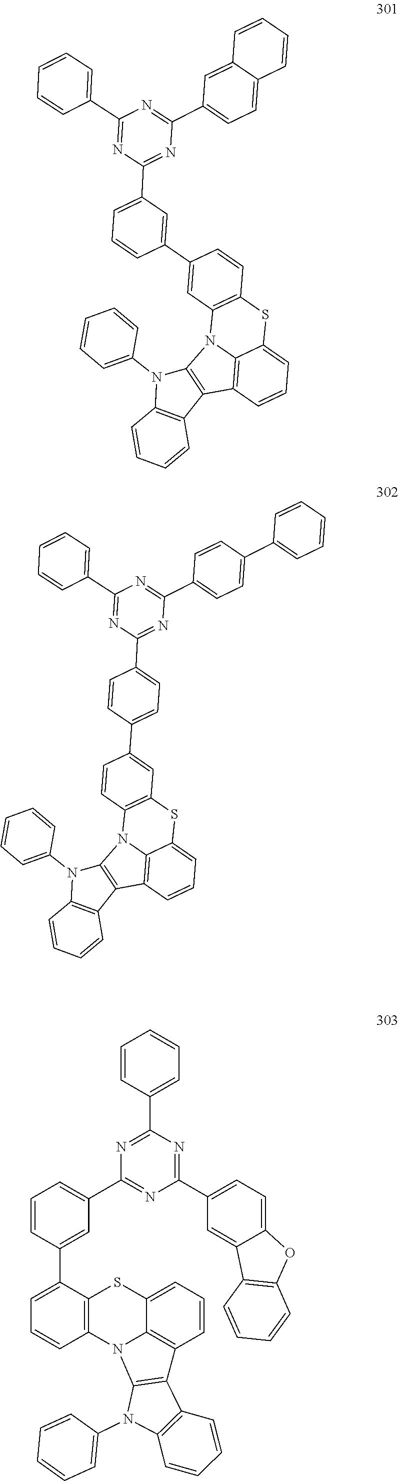 [SMILES: c1ccc(-c2nc(-c3cccc(-c4ccc5c(c4)-n4c6c(cccc6c6c7ccccc7n(-c7ccccc7)c64)S5)c3)nc(-c3ccc4ccccc4c3)n2)cc1 ; c1ccc(-c2ccc(-c3nc(-c4ccccc4)nc(-c4ccc(-c5ccc6c(c5)Sc5cccc7c8c9ccccc9n(-c9ccccc9)c8n-6c57)cc4)n3)cc2)cc1 ; c1ccc(-c2nc(-c3cccc(-c4cccc5c4Sc4cccc6c7c8ccccc8n(-c8ccccc8)c7n-5c46)c3)nc(-c3ccc4oc5ccccc5c4c3)n2)cc1]

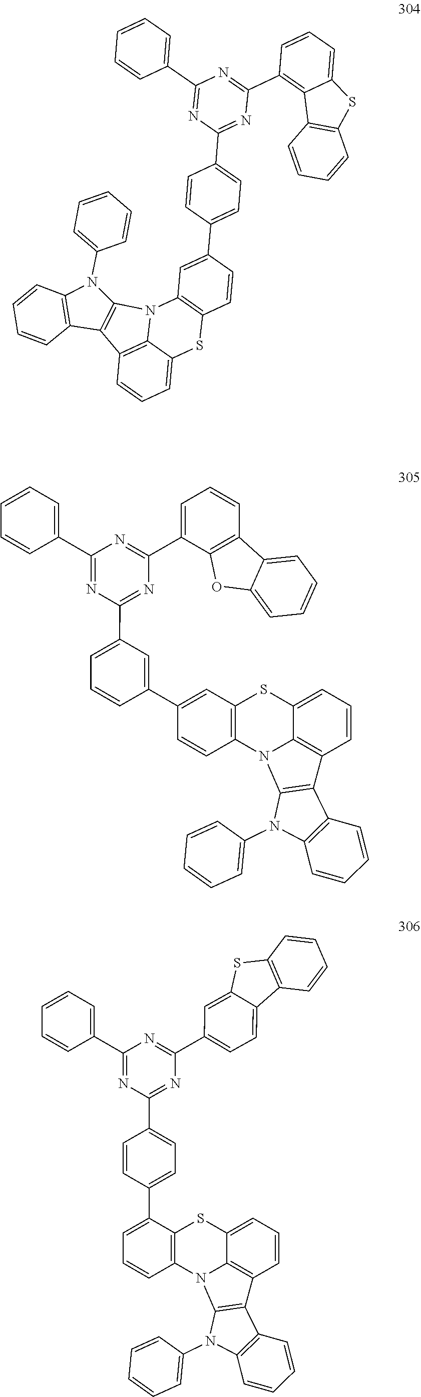 [SMILES: c1ccc(-c2nc(-c3ccc(-c4ccc5c(c4)-n4c6c(cccc6c6c7ccccc7n(-c7ccccc7)c64)S5)cc3)nc(-c3cccc4sc5ccccc5c34)n2)cc1 ; c1ccc(-c2nc(-c3cccc(-c4ccc5c(c4)Sc4cccc6c7c8ccccc8n(-c8ccccc8)c7n-5c46)c3)nc(-c3cccc4c3oc3ccccc34)n2)cc1 ; c1ccc(-c2nc(-c3ccc(-c4cccc5c4Sc4cccc6c7c8ccccc8n(-c8ccccc8)c7n-5c46)cc3)nc(-c3ccc4c(c3)sc3ccccc34)n2)cc1]

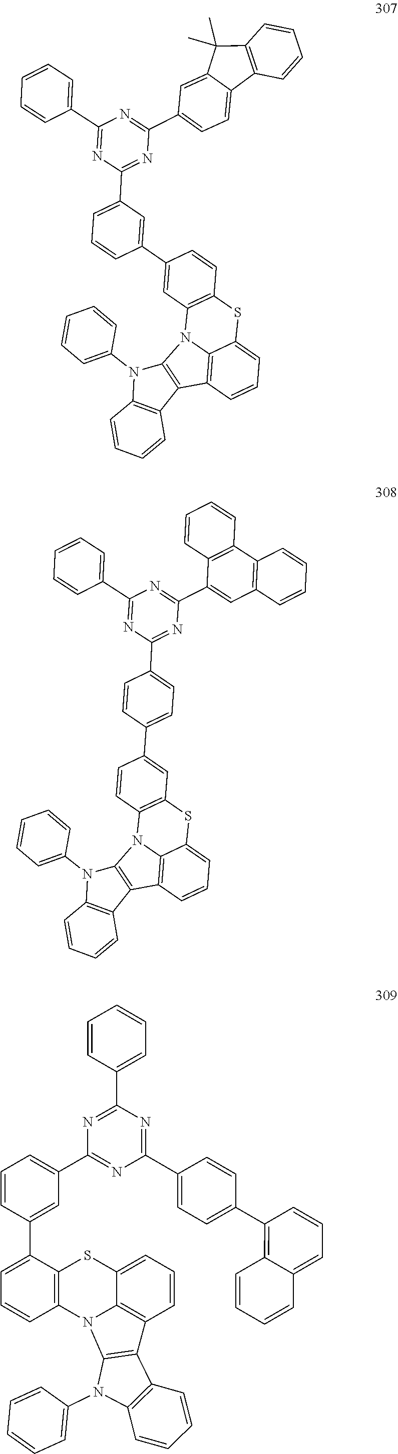 [SMILES: CC1(C)c2ccccc2-c2ccc(-c3nc(-c4ccccc4)nc(-c4cccc(-c5ccc6c(c5)-n5c7c(cccc7c7c8ccccc8n(-c8ccccc8)c75)S6)c4)n3)cc21 ; c1ccc(-c2nc(-c3ccc(-c4ccc5c(c4)Sc4cccc6c7c8ccccc8n(-c8ccccc8)c7n-5c46)cc3)nc(-c3cc4ccccc4c4ccccc34)n2)cc1 ; c1ccc(-c2nc(-c3ccc(-c4cccc5ccccc45)cc3)nc(-c3cccc(-c4cccc5c4Sc4cccc6c7c8ccccc8n(-c8ccccc8)c7n-5c46)c3)n2)cc1]

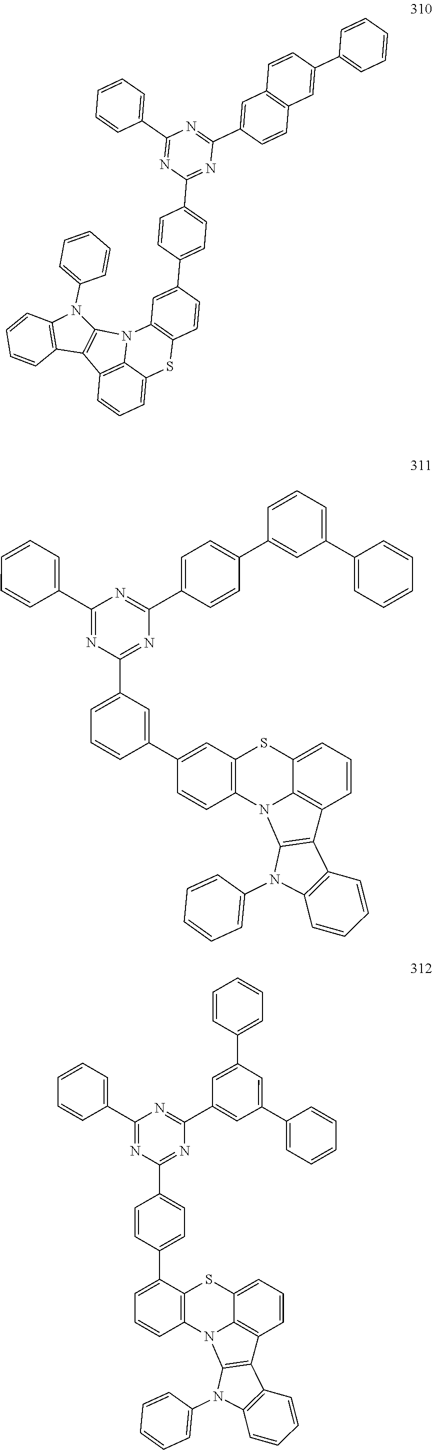 [SMILES: c1ccc(-c2ccc3cc(-c4nc(-c5ccccc5)nc(-c5ccc(-c6ccc7c(c6)-n6c8c(cccc8c8c9ccccc9n(-c9ccccc9)c86)S7)cc5)n4)ccc3c2)cc1 ; c1ccc(-c2cccc(-c3ccc(-c4nc(-c5ccccc5)nc(-c5cccc(-c6ccc7c(c6)Sc6cccc8c9c%10ccccc%10n(-c%10ccccc%10)c9n-7c68)c5)n4)cc3)c2)cc1 ; c1ccc(-c2cc(-c3ccccc3)cc(-c3nc(-c4ccccc4)nc(-c4ccc(-c5cccc6c5Sc5cccc7c8c9ccccc9n(-c9ccccc9)c8n-6c57)cc4)n3)c2)cc1]

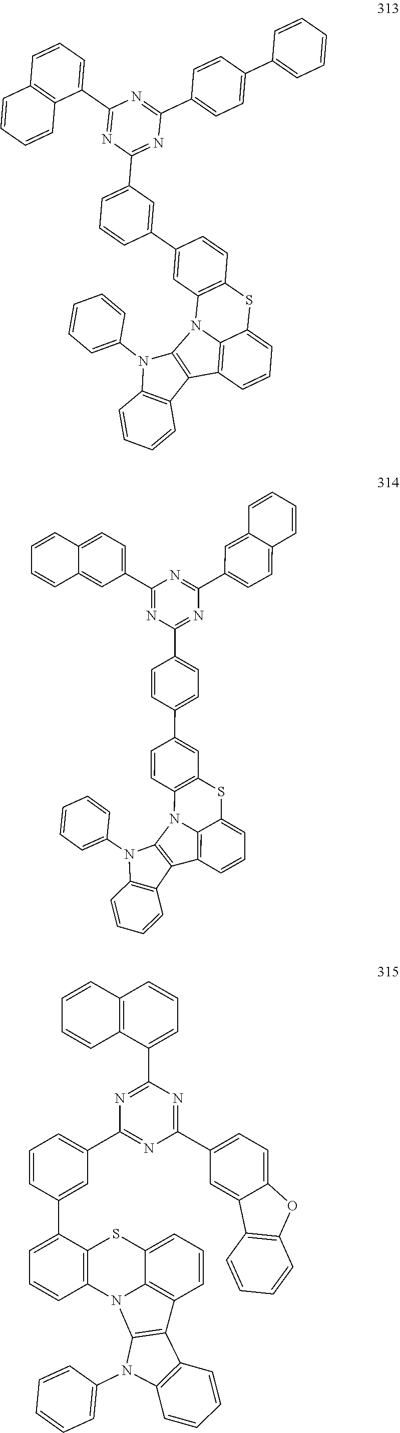 [SMILES: c1ccc(-c2ccc(-c3nc(-c4cccc(-c5ccc6c(c5)-n5c7c(cccc7c7c8ccccc8n(-c8ccccc8)c75)S6)c4)nc(-c4cccc5ccccc45)n3)cc2)cc1 ; c1ccc(-n2c3ccccc3c3c4cccc5c4n(c32)-c2ccc(-c3ccc(-c4nc(-c6ccc7ccccc7c6)nc(-c6ccc7ccccc7c6)n4)cc3)cc2S5)cc1 ; c1ccc(-n2c3ccccc3c3c4cccc5c4n(c32)-c2cccc(-c3cccc(-c4nc(-c6ccc7oc8ccccc8c7c6)nc(-c6cccc7ccccc67)n4)c3)c2S5)cc1]

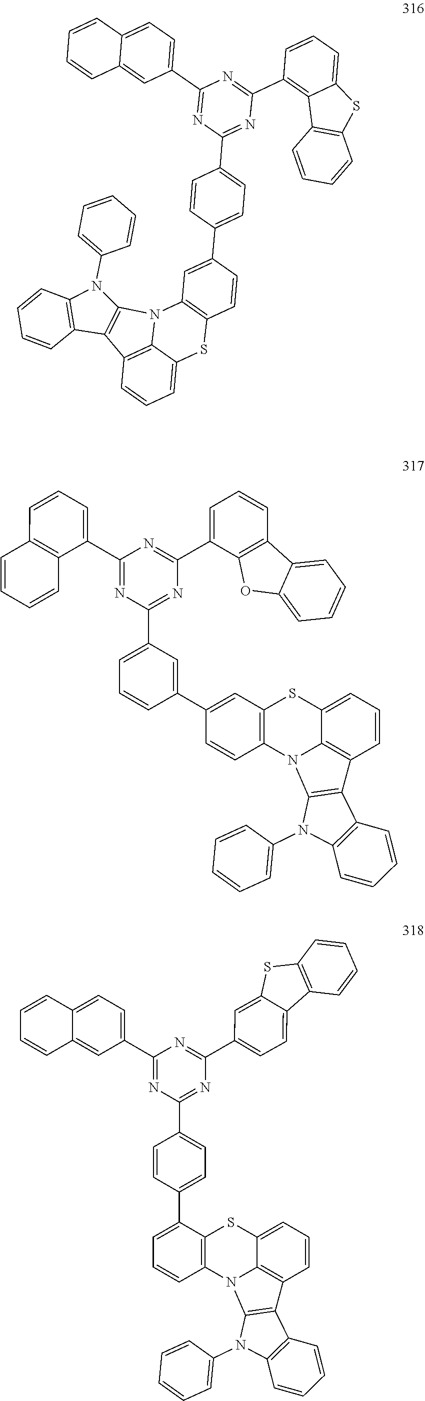 [SMILES: c1ccc(-n2c3ccccc3c3c4cccc5c4n(c32)-c2cc(-c3ccc(-c4nc(-c6ccc7ccccc7c6)nc(-c6cccc7sc8ccccc8c67)n4)cc3)ccc2S5)cc1 ; c1ccc(-n2c3ccccc3c3c4cccc5c4n(c32)-c2ccc(-c3cccc(-c4nc(-c6cccc7ccccc67)nc(-c6cccc7c6oc6ccccc67)n4)c3)cc2S5)cc1 ; c1ccc(-n2c3ccccc3c3c4cccc5c4n(c32)-c2cccc(-c3ccc(-c4nc(-c6ccc7ccccc7c6)nc(-c6ccc7c(c6)sc6ccccc67)n4)cc3)c2S5)cc1]

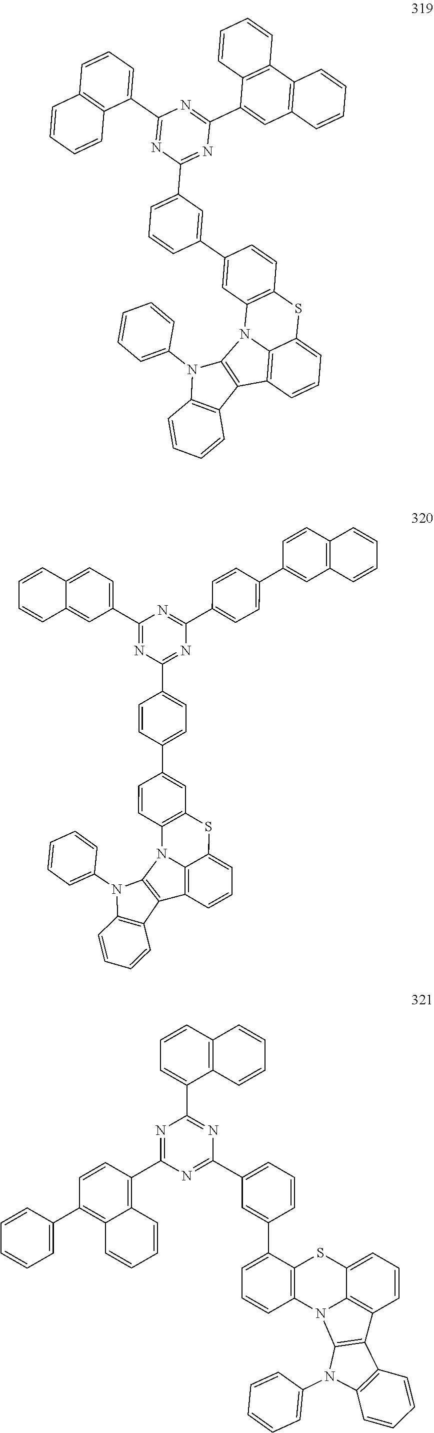 [SMILES: c1ccc(-n2c3ccccc3c3c4cccc5c4n(c32)-c2cc(-c3cccc(-c4nc(-c6cccc7ccccc67)nc(-c6cc7ccccc7c7ccccc67)n4)c3)ccc2S5)cc1 ; c1ccc(-n2c3ccccc3c3c4cccc5c4n(c32)-c2ccc(-c3ccc(-c4nc(-c6ccc(-c7ccc8ccccc8c7)cc6)nc(-c6ccc7ccccc7c6)n4)cc3)cc2S5)cc1 ; c1ccc(-c2ccc(-c3nc(-c4cccc(-c5cccc6c5Sc5cccc7c8c9ccccc9n(-c9ccccc9)c8n-6c57)c4)nc(-c4cccc5ccccc45)n3)c3ccccc23)cc1]

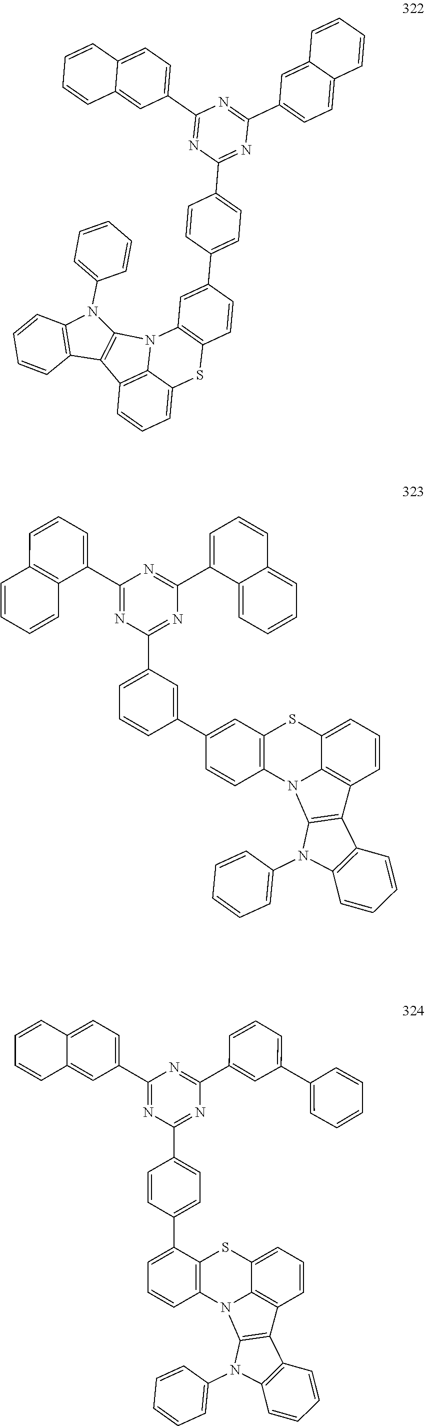 [SMILES: c1ccc(-n2c3ccccc3c3c4cccc5c4n(c32)-c2cc(-c3ccc(-c4nc(-c6ccc7ccccc7c6)nc(-c6ccc7ccccc7c6)n4)cc3)ccc2S5)cc1 ; c1ccc(-n2c3ccccc3c3c4cccc5c4n(c32)-c2ccc(-c3cccc(-c4nc(-c6cccc7ccccc67)nc(-c6cccc7ccccc67)n4)c3)cc2S5)cc1 ; c1ccc(-c2cccc(-c3nc(-c4ccc(-c5cccc6c5Sc5cccc7c8c9ccccc9n(-c9ccccc9)c8n-6c57)cc4)nc(-c4ccc5ccccc5c4)n3)c2)cc1]

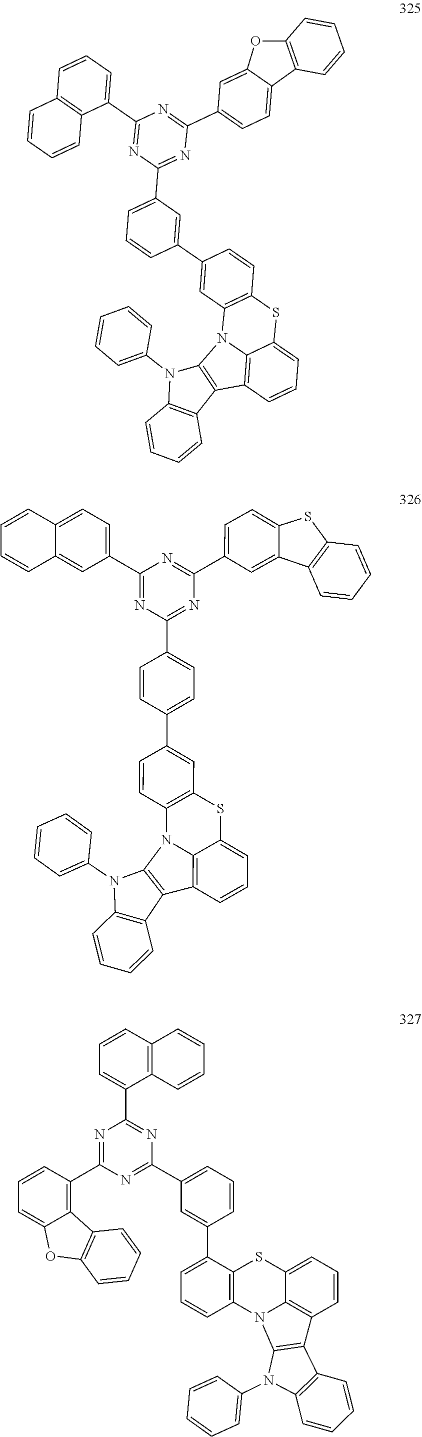 [SMILES: c1ccc(-n2c3ccccc3c3c4cccc5c4n(c32)-c2cc(-c3cccc(-c4nc(-c6ccc7c(c6)oc6ccccc67)nc(-c6cccc7ccccc67)n4)c3)ccc2S5)cc1 ; c1ccc(-n2c3ccccc3c3c4cccc5c4n(c32)-c2ccc(-c3ccc(-c4nc(-c6ccc7ccccc7c6)nc(-c6ccc7sc8ccccc8c7c6)n4)cc3)cc2S5)cc1 ; c1ccc(-n2c3ccccc3c3c4cccc5c4n(c32)-c2cccc(-c3cccc(-c4nc(-c6cccc7ccccc67)nc(-c6cccc7oc8ccccc8c67)n4)c3)c2S5)cc1]

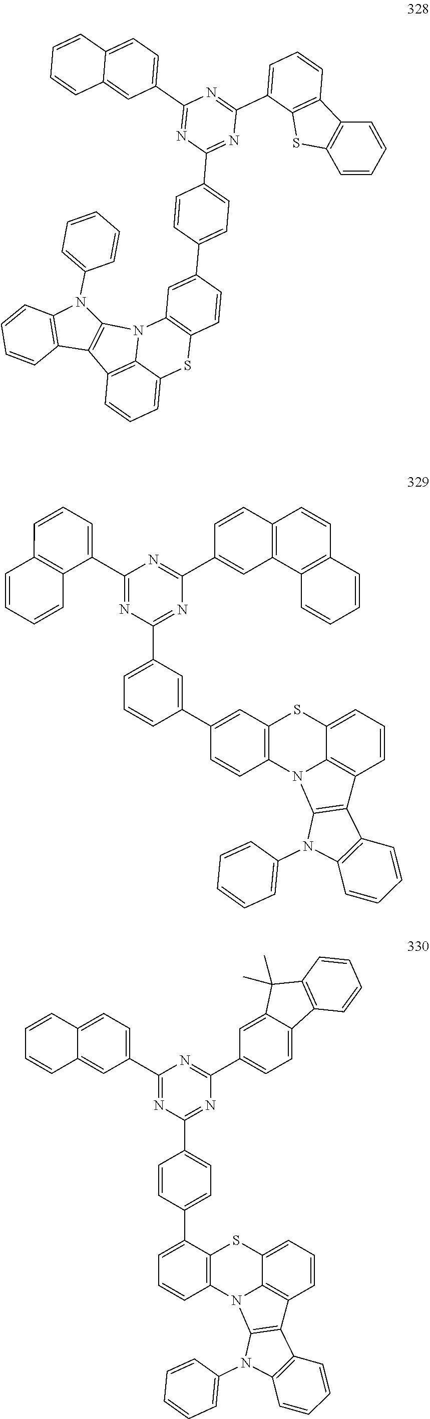 [SMILES: c1ccc(-n2c3ccccc3c3c4cccc5c4n(c32)-c2cc(-c3ccc(-c4nc(-c6ccc7ccccc7c6)nc(-c6cccc7c6sc6ccccc67)n4)cc3)ccc2S5)cc1 ; c1ccc(-n2c3ccccc3c3c4cccc5c4n(c32)-c2ccc(-c3cccc(-c4nc(-c6ccc7ccc8ccccc8c7c6)nc(-c6cccc7ccccc67)n4)c3)cc2S5)cc1 ; CC1(C)c2ccccc2-c2ccc(-c3nc(-c4ccc(-c5cccc6c5Sc5cccc7c8c9ccccc9n(-c9ccccc9)c8n-6c57)cc4)nc(-c4ccc5ccccc5c4)n3)cc21]

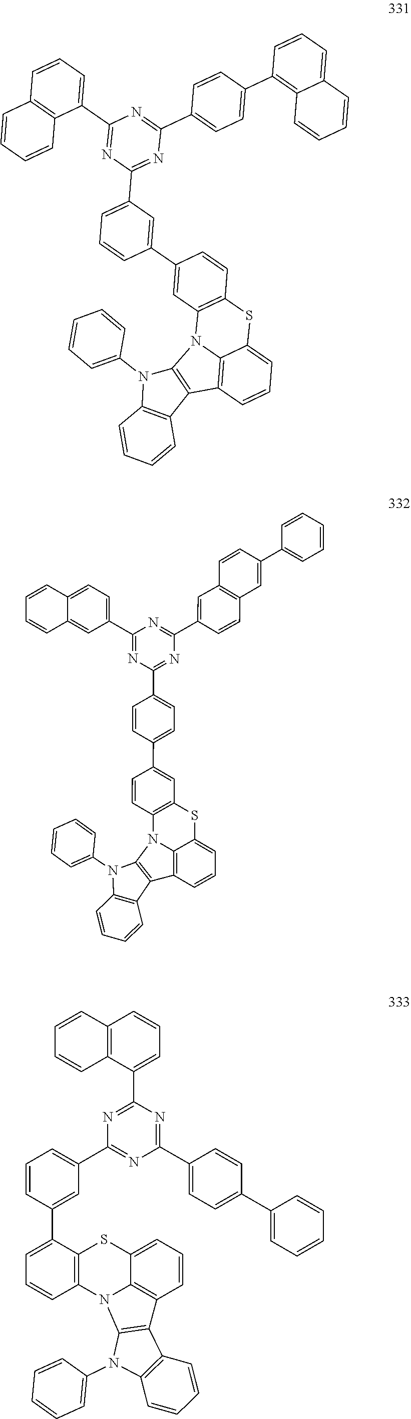 [SMILES: c1ccc(-n2c3ccccc3c3c4cccc5c4n(c32)-c2cc(-c3cccc(-c4nc(-c6ccc(-c7cccc8ccccc78)cc6)nc(-c6cccc7ccccc67)n4)c3)ccc2S5)cc1 ; c1ccc(-c2ccc3cc(-c4nc(-c5ccc(-c6ccc7c(c6)Sc6cccc8c9c%10ccccc%10n(-c%10ccccc%10)c9n-7c68)cc5)nc(-c5ccc6ccccc6c5)n4)ccc3c2)cc1 ; c1ccc(-c2ccc(-c3nc(-c4cccc(-c5cccc6c5Sc5cccc7c8c9ccccc9n(-c9ccccc9)c8n-6c57)c4)nc(-c4cccc5ccccc45)n3)cc2)cc1]

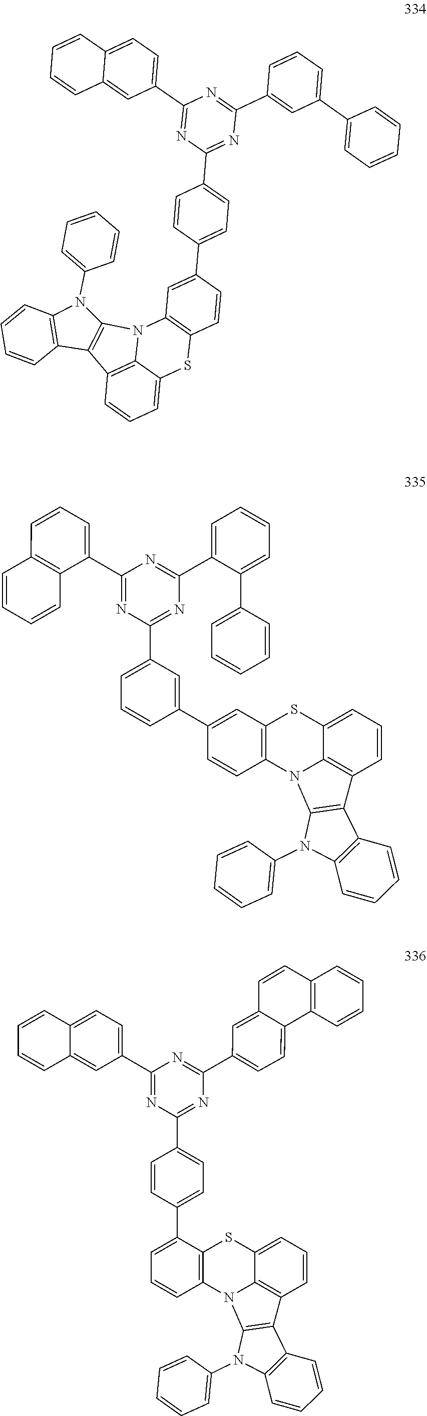 [SMILES: c1ccc(-c2cccc(-c3nc(-c4ccc(-c5ccc6c(c5)-n5c7c(cccc7c7c8ccccc8n(-c8ccccc8)c75)S6)cc4)nc(-c4ccc5ccccc5c4)n3)c2)cc1 ; c1ccc(-c2ccccc2-c2nc(-c3cccc(-c4ccc5c(c4)Sc4cccc6c7c8ccccc8n(-c8ccccc8)c7n-5c46)c3)nc(-c3cccc4ccccc34)n2)cc1 ; c1ccc(-n2c3ccccc3c3c4cccc5c4n(c32)-c2cccc(-c3ccc(-c4nc(-c6ccc7ccccc7c6)nc(-c6ccc7c(ccc8ccccc87)c6)n4)cc3)c2S5)cc1]

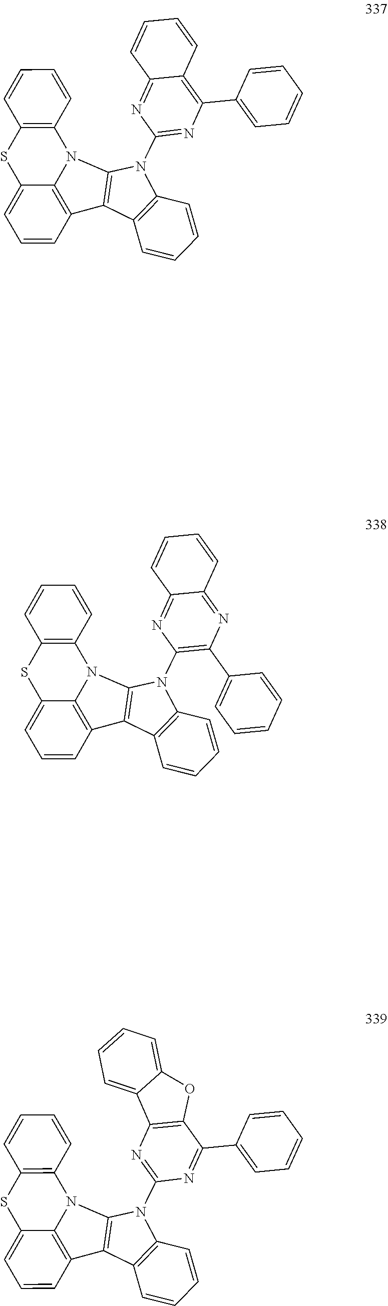 [SMILES: c1ccc(-c2nc(-n3c4ccccc4c4c5cccc6c5n(c43)-c3ccccc3S6)nc3ccccc23)cc1 ; c1ccc(-c2nc3ccccc3nc2-n2c3ccccc3c3c4cccc5c4n(c32)-c2ccccc2S5)cc1 ; c1ccc(-c2nc(-n3c4ccccc4c4c5cccc6c5n(c43)-c3ccccc3S6)nc3c2oc2ccccc23)cc1]

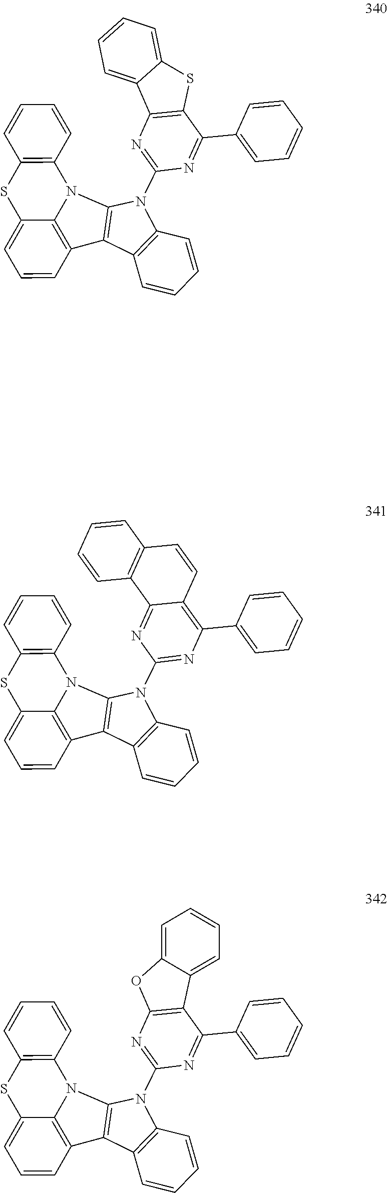 [SMILES: c1ccc(-c2nc(-n3c4ccccc4c4c5cccc6c5n(c43)-c3ccccc3S6)nc3c2sc2ccccc23)cc1 ; c1ccc(-c2nc(-n3c4ccccc4c4c5cccc6c5n(c43)-c3ccccc3S6)nc3c2ccc2ccccc23)cc1 ; c1ccc(-c2nc(-n3c4ccccc4c4c5cccc6c5n(c43)-c3ccccc3S6)nc3oc4ccccc4c23)cc1]

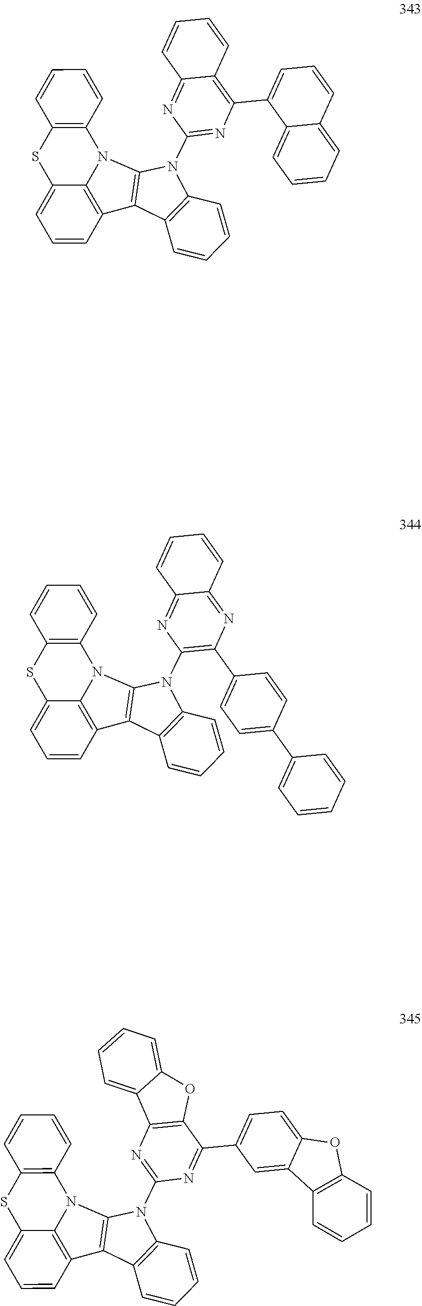 [SMILES: c1ccc2c(c1)Sc1cccc3c4c5ccccc5n(-c5nc(-c6cccc7ccccc67)c6ccccc6n5)c4n-2c13 ; c1ccc(-c2ccc(-c3nc4ccccc4nc3-n3c4ccccc4c4c5cccc6c5n(c43)-c3ccccc3S6)cc2)cc1 ; c1ccc2c(c1)Sc1cccc3c4c5ccccc5n(-c5nc(-c6ccc7oc8ccccc8c7c6)c6oc7ccccc7c6n5)c4n-2c13]

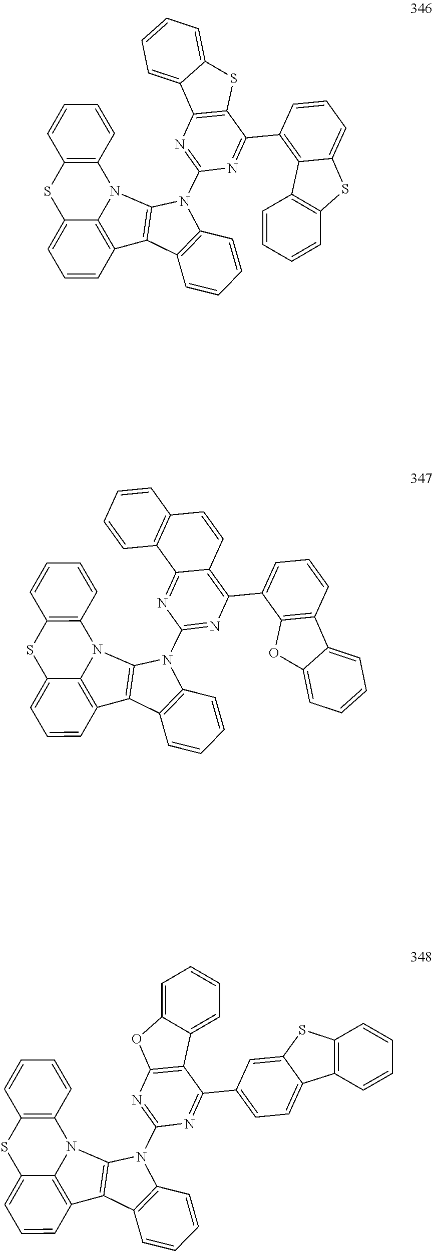 [SMILES: c1ccc2c(c1)Sc1cccc3c4c5ccccc5n(-c5nc(-c6cccc7sc8ccccc8c67)c6sc7ccccc7c6n5)c4n-2c13 ; c1ccc2c(c1)Sc1cccc3c4c5ccccc5n(-c5nc(-c6cccc7c6oc6ccccc67)c6ccc7ccccc7c6n5)c4n-2c13 ; c1ccc2c(c1)Sc1cccc3c4c5ccccc5n(-c5nc(-c6ccc7c(c6)sc6ccccc67)c6c(n5)oc5ccccc56)c4n-2c13]

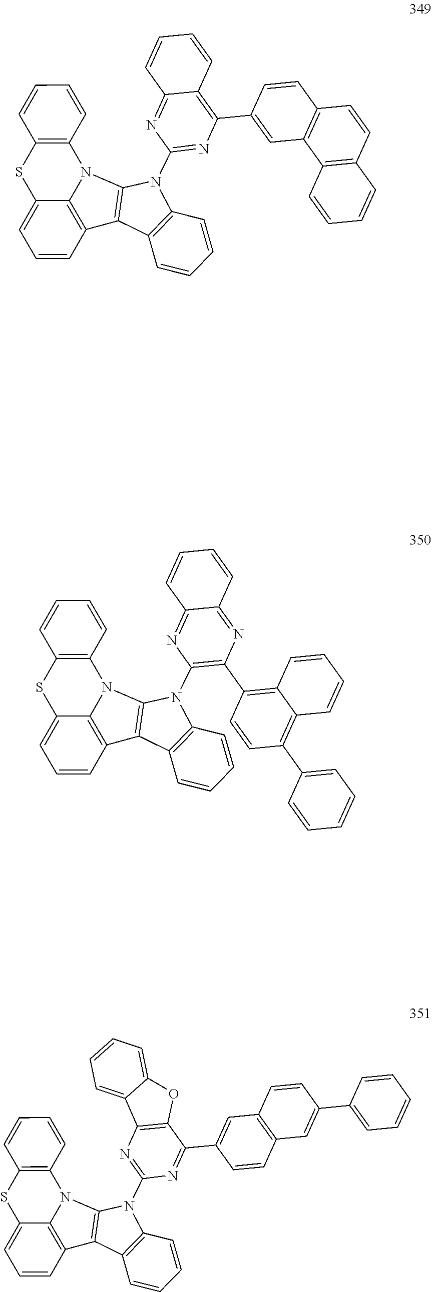 [SMILES: c1ccc2c(c1)Sc1cccc3c4c5ccccc5n(-c5nc(-c6ccc7ccc8ccccc8c7c6)c6ccccc6n5)c4n-2c13 ; c1ccc(-c2ccc(-c3nc4ccccc4nc3-n3c4ccccc4c4c5cccc6c5n(c43)-c3ccccc3S6)c3ccccc23)cc1 ; c1ccc(-c2ccc3cc(-c4nc(-n5c6ccccc6c6c7cccc8c7n(c65)-c5ccccc5S8)nc5c4oc4ccccc45)ccc3c2)cc1]

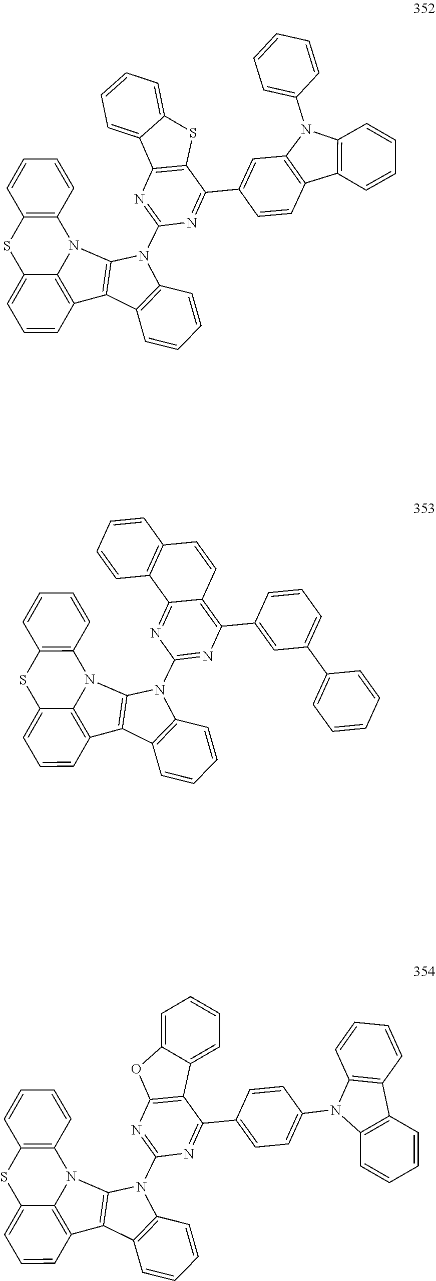 [SMILES: c1ccc(-n2c3ccccc3c3ccc(-c4nc(-n5c6ccccc6c6c7cccc8c7n(c65)-c5ccccc5S8)nc5c4sc4ccccc45)cc32)cc1 ; c1ccc(-c2cccc(-c3nc(-n4c5ccccc5c5c6cccc7c6n(c54)-c4ccccc4S7)nc4c3ccc3ccccc34)c2)cc1 ; c1ccc2c(c1)Sc1cccc3c4c5ccccc5n(-c5nc(-c6ccc(-n7c8ccccc8c8ccccc87)cc6)c6c(n5)oc5ccccc56)c4n-2c13]

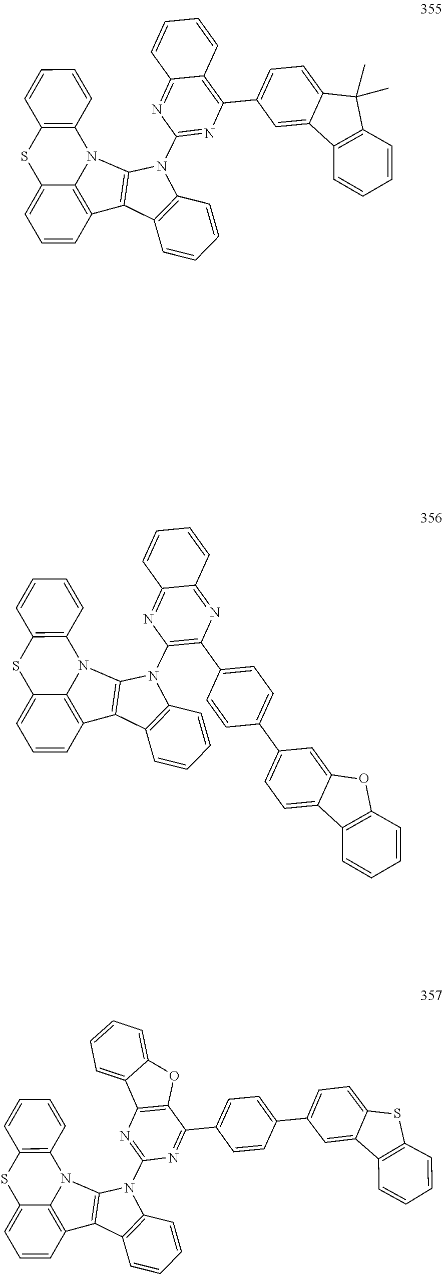 [SMILES: CC1(C)c2ccccc2-c2cc(-c3nc(-n4c5ccccc5c5c6cccc7c6n(c54)-c4ccccc4S7)nc4ccccc34)ccc21 ; c1ccc2c(c1)Sc1cccc3c4c5ccccc5n(-c5nc6ccccc6nc5-c5ccc(-c6ccc7c(c6)oc6ccccc67)cc5)c4n-2c13 ; c1ccc2c(c1)Sc1cccc3c4c5ccccc5n(-c5nc(-c6ccc(-c7ccc8sc9ccccc9c8c7)cc6)c6oc7ccccc7c6n5)c4n-2c13]

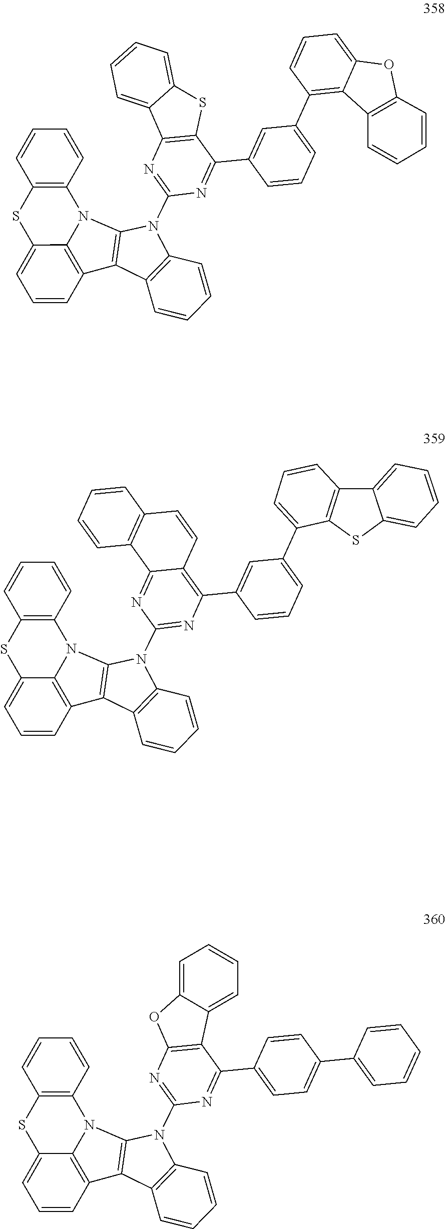 [SMILES: c1cc(-c2nc(-n3c4ccccc4c4c5cccc6c5n(c43)-c3ccccc3S6)nc3c2sc2ccccc23)cc(-c2cccc3oc4ccccc4c23)c1 ; c1cc(-c2nc(-n3c4ccccc4c4c5cccc6c5n(c43)-c3ccccc3S6)nc3c2ccc2ccccc23)cc(-c2cccc3c2sc2ccccc23)c1 ; c1ccc(-c2ccc(-c3nc(-n4c5ccccc5c5c6cccc7c6n(c54)-c4ccccc4S7)nc4oc5ccccc5c34)cc2)cc1]

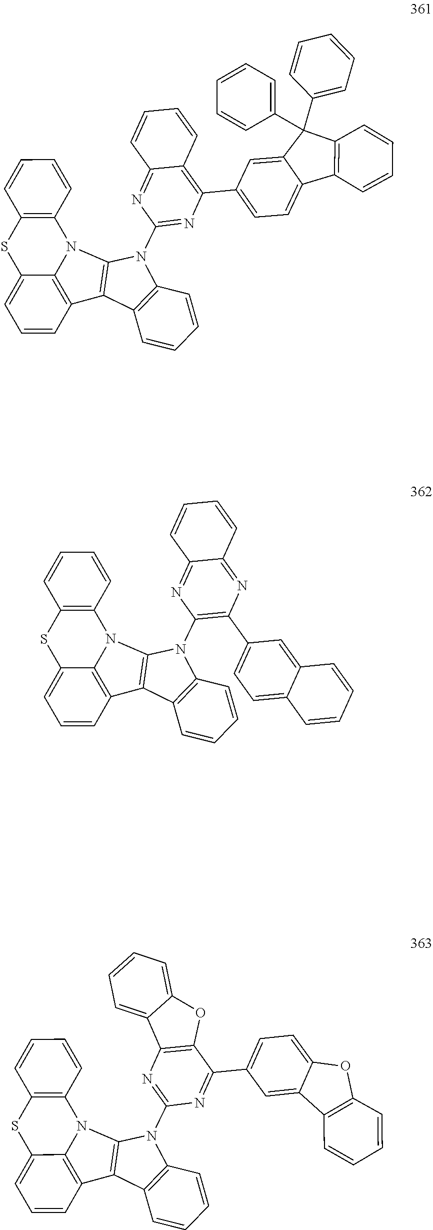 [SMILES: c1ccc(C2(c3ccccc3)c3ccccc3-c3ccc(-c4nc(-n5c6ccccc6c6c7cccc8c7n(c65)-c5ccccc5S8)nc5ccccc45)cc32)cc1 ; c1ccc2c(c1)Sc1cccc3c4c5ccccc5n(-c5nc6ccccc6nc5-c5ccc6ccccc6c5)c4n-2c13 ; c1ccc2c(c1)Sc1cccc3c4c5ccccc5n(-c5nc(-c6ccc7oc8ccccc8c7c6)c6oc7ccccc7c6n5)c4n-2c13]

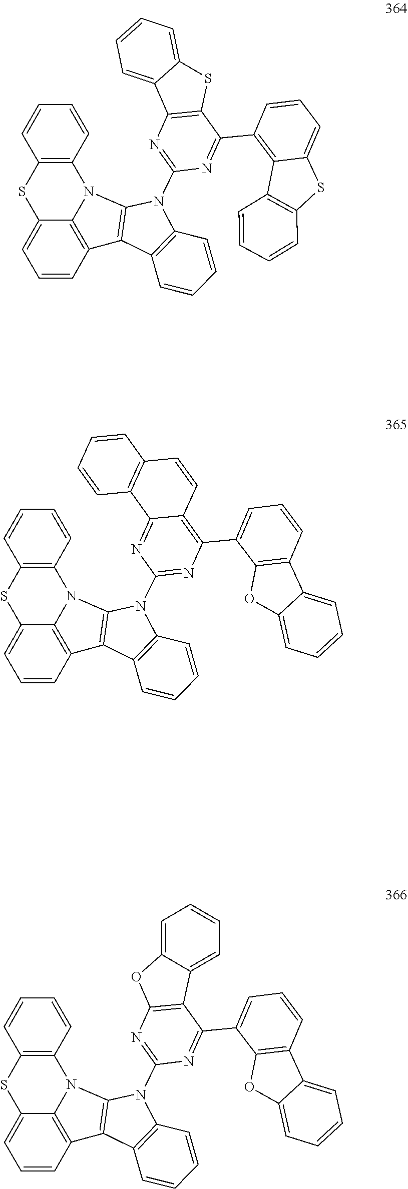 [SMILES: c1ccc2c(c1)Sc1cccc3c4c5ccccc5n(-c5nc(-c6cccc7sc8ccccc8c67)c6sc7ccccc7c6n5)c4n-2c13 ; c1ccc2c(c1)Sc1cccc3c4c5ccccc5n(-c5nc(-c6cccc7c6oc6ccccc67)c6ccc7ccccc7c6n5)c4n-2c13 ; c1ccc2c(c1)Sc1cccc3c4c5ccccc5n(-c5nc(-c6cccc7c6oc6ccccc67)c6c(n5)oc5ccccc56)c4n-2c13]

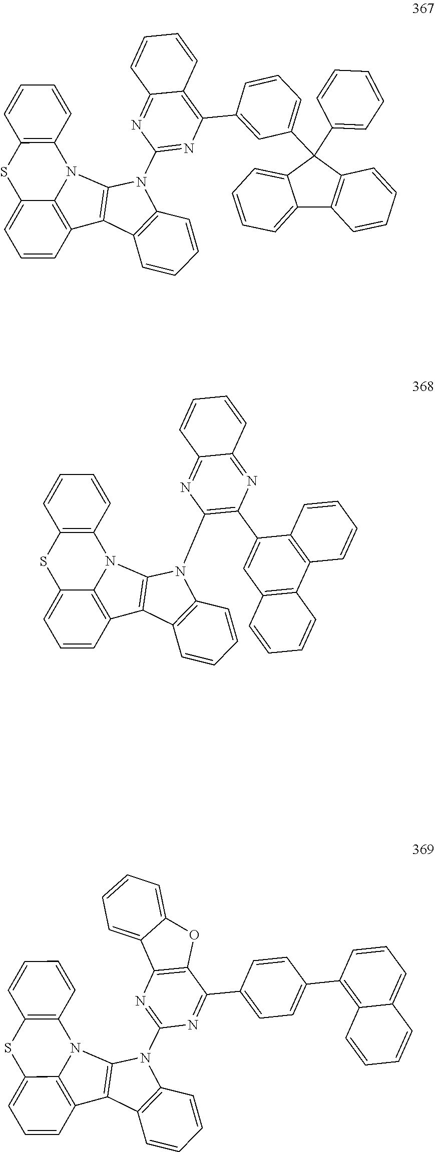 [SMILES: c1ccc(C2(c3cccc(-c4nc(-n5c6ccccc6c6c7cccc8c7n(c65)-c5ccccc5S8)nc5ccccc45)c3)c3ccccc3-c3ccccc32)cc1 ; c1ccc2c(c1)Sc1cccc3c4c5ccccc5n(-c5nc6ccccc6nc5-c5cc6ccccc6c6ccccc56)c4n-2c13 ; c1ccc2c(c1)Sc1cccc3c4c5ccccc5n(-c5nc(-c6ccc(-c7cccc8ccccc78)cc6)c6oc7ccccc7c6n5)c4n-2c13]

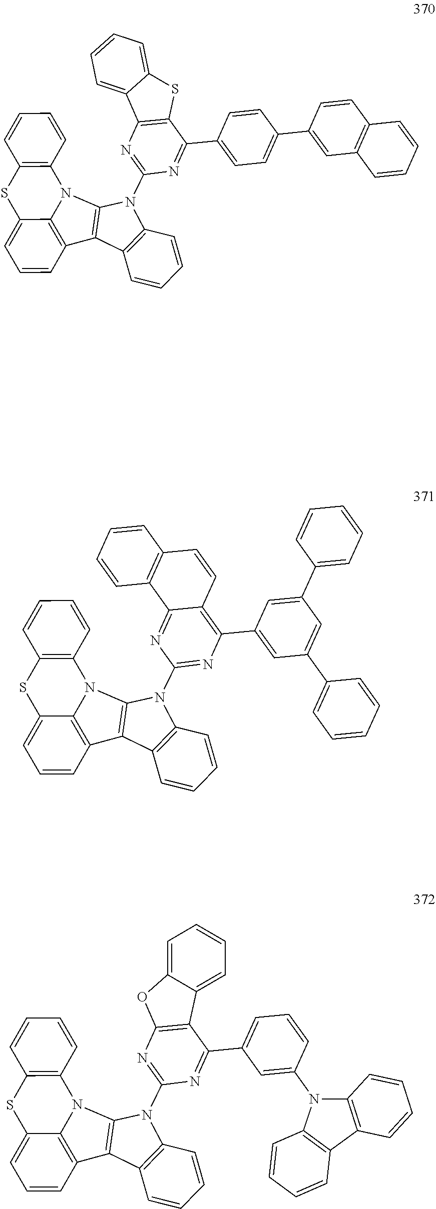 [SMILES: c1ccc2c(c1)Sc1cccc3c4c5ccccc5n(-c5nc(-c6ccc(-c7ccc8ccccc8c7)cc6)c6sc7ccccc7c6n5)c4n-2c13 ; c1ccc(-c2cc(-c3ccccc3)cc(-c3nc(-n4c5ccccc5c5c6cccc7c6n(c54)-c4ccccc4S7)nc4c3ccc3ccccc34)c2)cc1 ; c1cc(-c2nc(-n3c4ccccc4c4c5cccc6c5n(c43)-c3ccccc3S6)nc3oc4ccccc4c23)cc(-n2c3ccccc3c3ccccc32)c1]

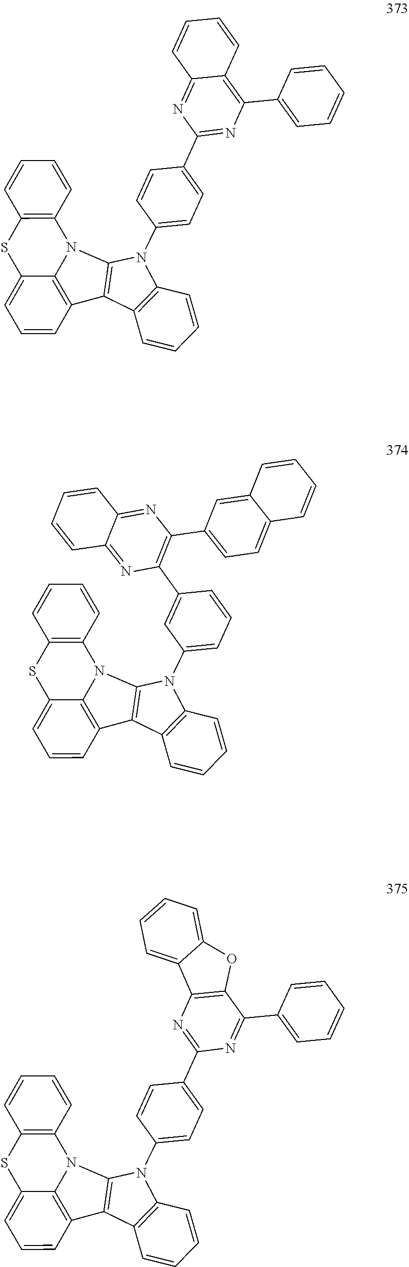 [SMILES: c1ccc(-c2nc(-c3ccc(-n4c5ccccc5c5c6cccc7c6n(c54)-c4ccccc4S7)cc3)nc3ccccc23)cc1 ; c1cc(-c2nc3ccccc3nc2-c2ccc3ccccc3c2)cc(-n2c3ccccc3c3c4cccc5c4n(c32)-c2ccccc2S5)c1 ; c1ccc(-c2nc(-c3ccc(-n4c5ccccc5c5c6cccc7c6n(c54)-c4ccccc4S7)cc3)nc3c2oc2ccccc23)cc1]

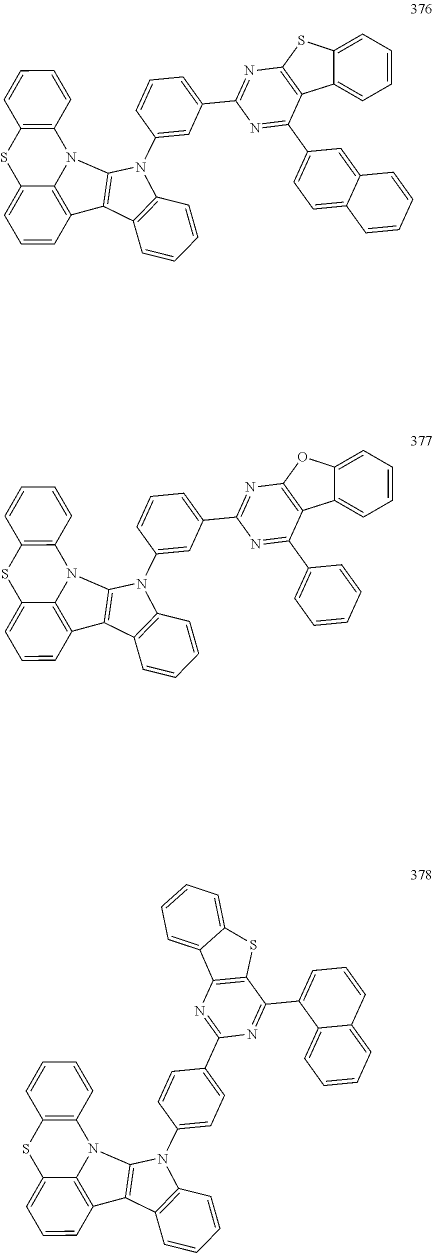 [SMILES: c1cc(-c2nc(-c3ccc4ccccc4c3)c3c(n2)sc2ccccc23)cc(-n2c3ccccc3c3c4cccc5c4n(c32)-c2ccccc2S5)c1 ; c1ccc(-c2nc(-c3cccc(-n4c5ccccc5c5c6cccc7c6n(c54)-c4ccccc4S7)c3)nc3oc4ccccc4c23)cc1 ; c1ccc2c(c1)Sc1cccc3c4c5ccccc5n(-c5ccc(-c6nc(-c7cccc8ccccc78)c7sc8ccccc8c7n6)cc5)c4n-2c13]

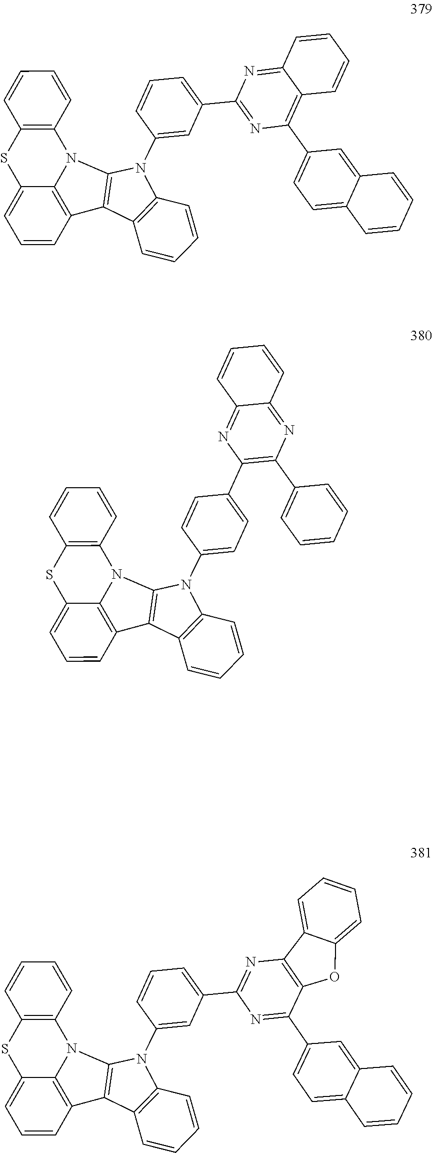 [SMILES: c1cc(-c2nc(-c3ccc4ccccc4c3)c3ccccc3n2)cc(-n2c3ccccc3c3c4cccc5c4n(c32)-c2ccccc2S5)c1 ; c1ccc(-c2nc3ccccc3nc2-c2ccc(-n3c4ccccc4c4c5cccc6c5n(c43)-c3ccccc3S6)cc2)cc1 ; c1cc(-c2nc(-c3ccc4ccccc4c3)c3oc4ccccc4c3n2)cc(-n2c3ccccc3c3c4cccc5c4n(c32)-c2ccccc2S5)c1]

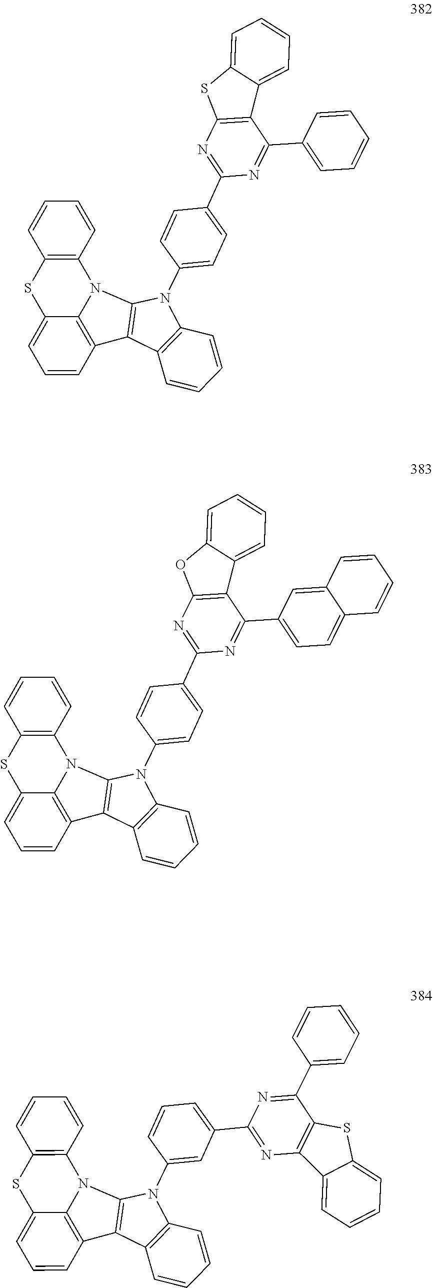 [SMILES: c1ccc(-c2nc(-c3ccc(-n4c5ccccc5c5c6cccc7c6n(c54)-c4ccccc4S7)cc3)nc3sc4ccccc4c23)cc1 ; c1ccc2c(c1)Sc1cccc3c4c5ccccc5n(-c5ccc(-c6nc(-c7ccc8ccccc8c7)c7c(n6)oc6ccccc67)cc5)c4n-2c13 ; c1ccc(-c2nc(-c3cccc(-n4c5ccccc5c5c6cccc7c6n(c54)-c4ccccc4S7)c3)nc3c2sc2ccccc23)cc1]

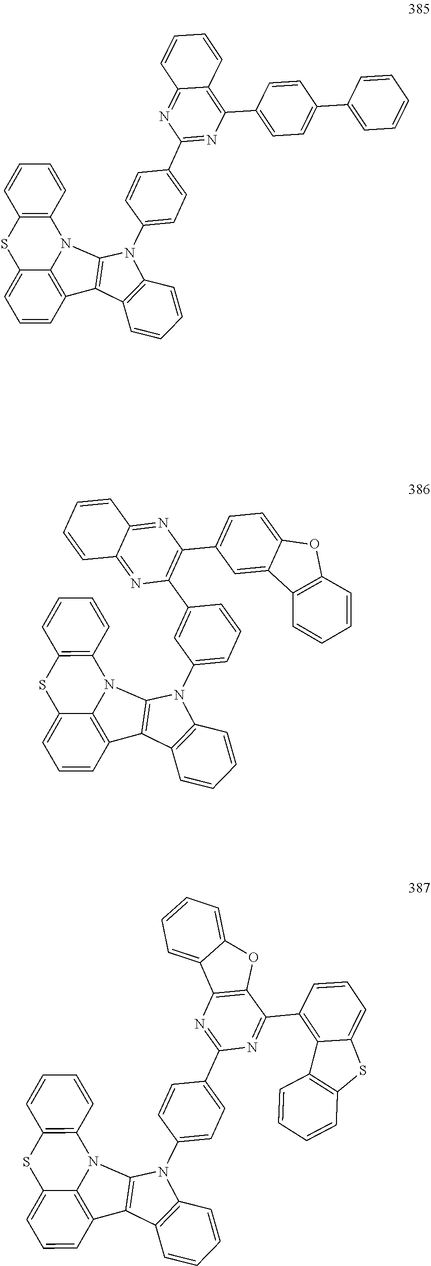 [SMILES: c1ccc(-c2ccc(-c3nc(-c4ccc(-n5c6ccccc6c6c7cccc8c7n(c65)-c5ccccc5S8)cc4)nc4ccccc34)cc2)cc1 ; c1cc(-c2nc3ccccc3nc2-c2ccc3oc4ccccc4c3c2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7cccc8sc9ccccc9c78)c7oc8ccccc8c7n6)cc5)c4n-2c13]

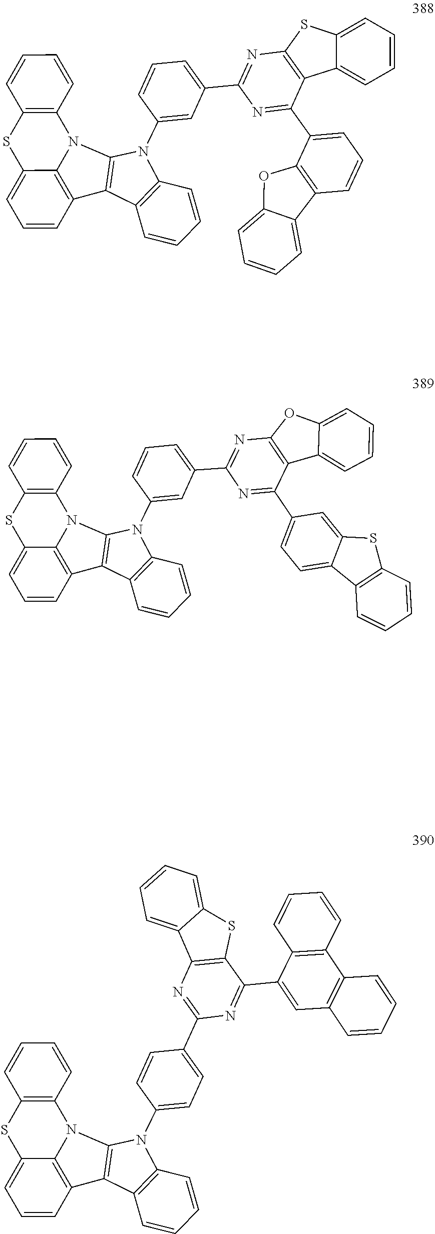 [SMILES: c1cc(-c2nc(-c3cccc4c3oc3ccccc34)c3c(n2)sc2ccccc23)cc(-n2c3ccccc3c3c4cccc5c4n(c32)-c2ccccc2S5)c1 ; c1cc(-c2nc(-c3ccc4c(c3)sc3ccccc34)c3c(n2)oc2ccccc23)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7cc8ccccc8c8ccccc78)c7sc8ccccc8c7n6)cc5)c4n-2c13]

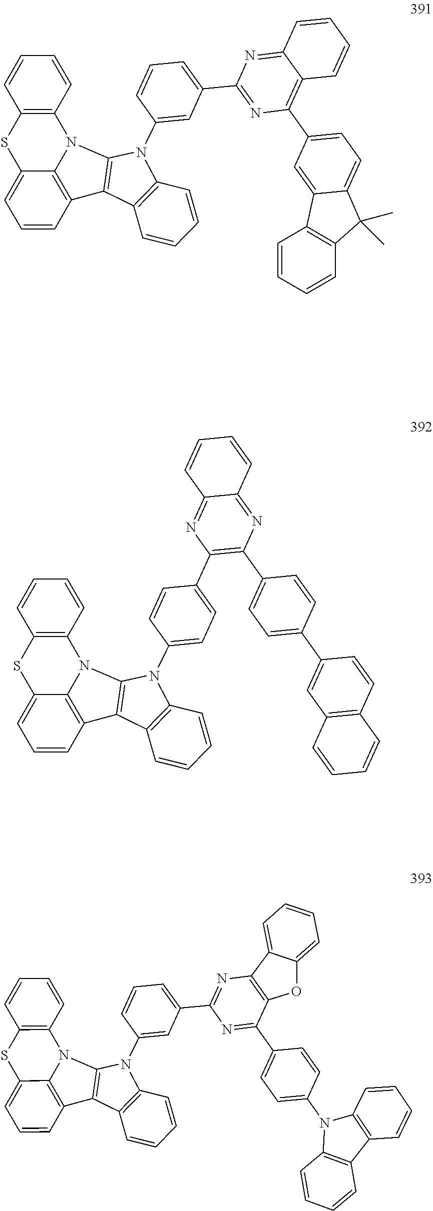 [SMILES: CC1(C)c2ccccc2-c2cc(-c3nc(-c4cccc(-n5c6ccccc6c6c7cccc8c7n(c65)-c5ccccc5S8)c4)nc4ccccc34)ccc21 ; c1ccc2c(c1)Sc1cccc3c4c5ccccc5n(-c5ccc(-c6nc7ccccc7nc6-c6ccc(-c7ccc8ccccc8c7)cc6)cc5)c4n-2c13 ; c1cc(-c2nc(-c3ccc(-n4c5ccccc5c5ccccc54)cc3)c3oc4ccccc4c3n2)cc(-n2c3ccccc3c3c4cccc5c4n(c32)-c2ccccc2S5)c1]

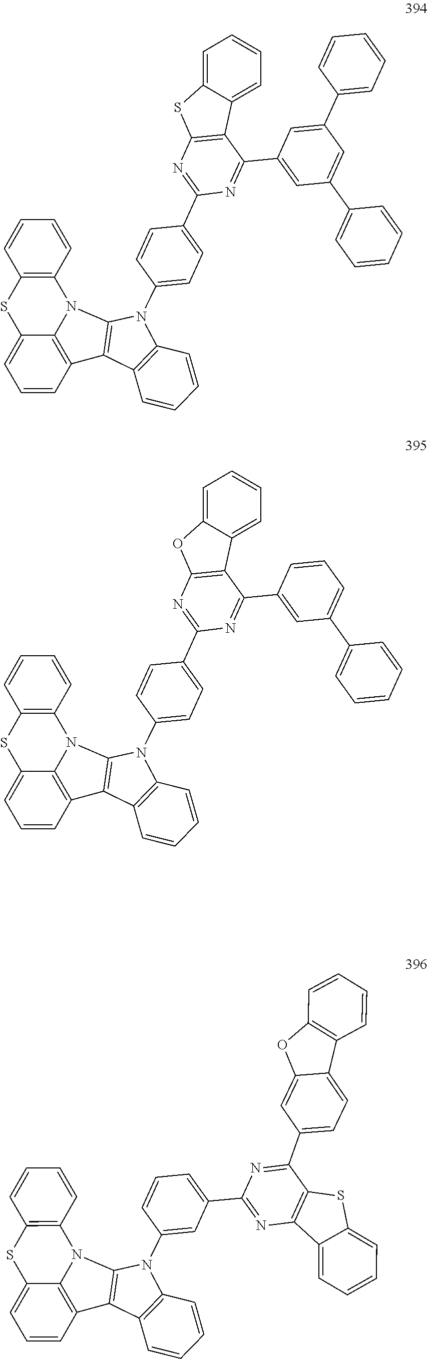 [SMILES: c1ccc(-c2cc(-c3ccccc3)cc(-c3nc(-c4ccc(-n5c6ccccc6c6c7cccc8c7n(c65)-c5ccccc5S8)cc4)nc4sc5ccccc5c34)c2)cc1 ; c1ccc(-c2cccc(-c3nc(-c4ccc(-n5c6ccccc6c6c7cccc8c7n(c65)-c5ccccc5S8)cc4)nc4oc5ccccc5c34)c2)cc1 ; c1cc(-c2nc(-c3ccc4c(c3)oc3ccccc34)c3sc4ccccc4c3n2)cc(-n2c3ccccc3c3c4cccc5c4n(c32)-c2ccccc2S5)c1]

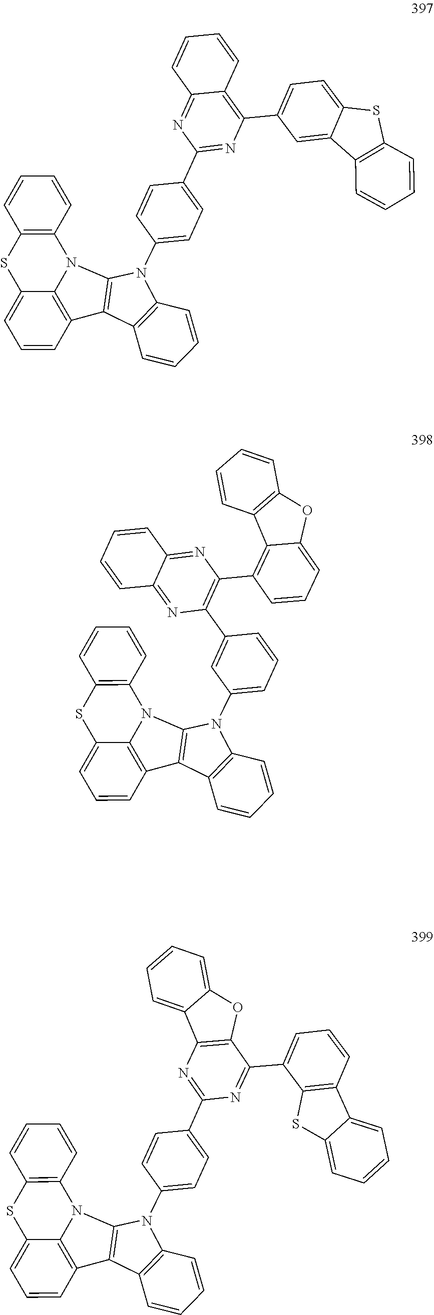 [SMILES: c1ccc2c(c1)Sc1cccc3c4c5ccccc5n(-c5ccc(-c6nc(-c7ccc8sc9ccccc9c8c7)c7ccccc7n6)cc5)c4n-2c13 ; c1cc(-c2nc3ccccc3nc2-c2cccc3oc4ccccc4c23)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7cccc8c7sc7ccccc78)c7oc8ccccc8c7n6)cc5)c4n-2c13]

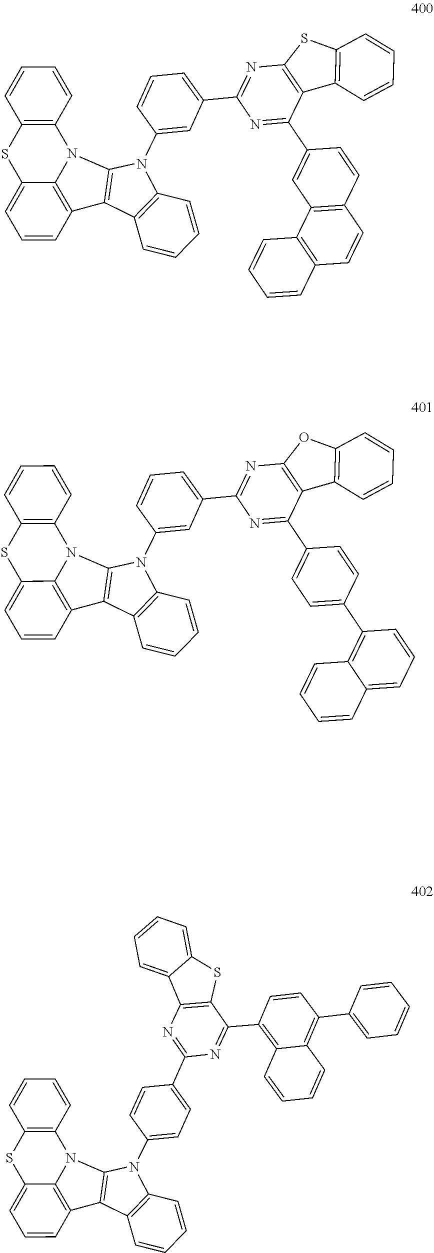 [SMILES: c1cc(-c2nc(-c3ccc4ccc5ccccc5c4c3)c3c(n2)sc2ccccc23)cc(-n2c3ccccc3c3c4cccc5c4n(c32)-c2ccccc2S5)c1 ; c1cc(-c2nc(-c3ccc(-c4cccc5ccccc45)cc3)c3c(n2)oc2ccccc23)cc(-n2c3ccccc3c3c4cccc5c4n(c32)-c2ccccc2S5)c1 ; c1ccc(-c2ccc(-c3nc(-c4ccc(-n5c6ccccc6c6c7cccc8c7n(c65)-c5ccccc5S8)cc4)nc4c3sc3ccccc34)c3ccccc23)cc1]

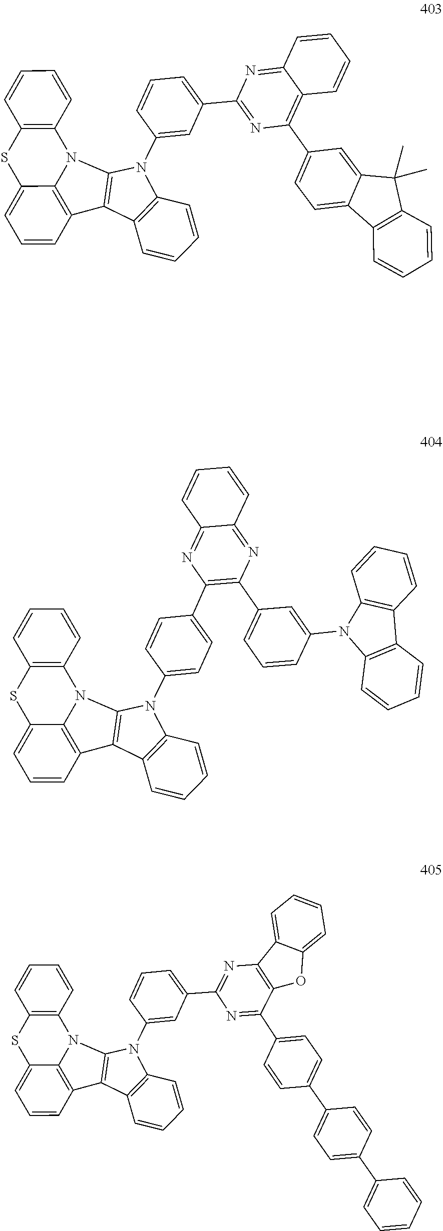 [SMILES: CC1(C)c2ccccc2-c2ccc(-c3nc(-c4cccc(-n5c6ccccc6c6c7cccc8c7n(c65)-c5ccccc5S8)c4)nc4ccccc34)cc21 ; c1cc(-c2nc3ccccc3nc2-c2ccc(-n3c4ccccc4c4c5cccc6c5n(c43)-c3ccccc3S6)cc2)cc(-n2c3ccccc3c3ccccc32)c1 ; c1ccc(-c2ccc(-c3ccc(-c4nc(-c5cccc(-n6c7ccccc7c7c8cccc9c8n(c76)-c6ccccc6S9)c5)nc5c4oc4ccccc45)cc3)cc2)cc1]

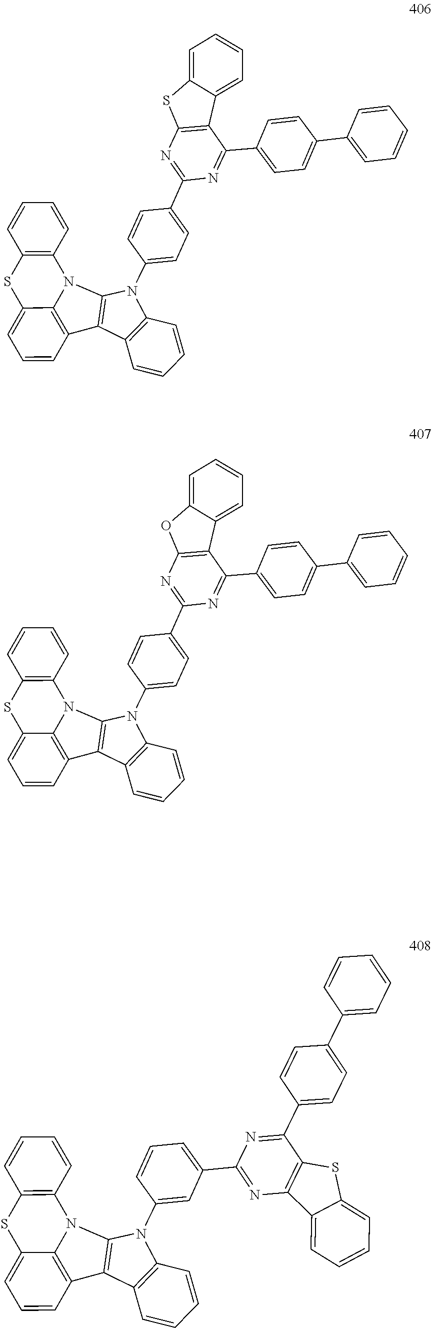 [SMILES: c1ccc(-c2ccc(-c3nc(-c4ccc(-n5c6ccccc6c6c7cccc8c7n(c65)-c5ccccc5S8)cc4)nc4sc5ccccc5c34)cc2)cc1 ; c1ccc(-c2ccc(-c3nc(-c4ccc(-n5c6ccccc6c6c7cccc8c7n(c65)-c5ccccc5S8)cc4)nc4oc5ccccc5c34)cc2)cc1 ; c1ccc(-c2ccc(-c3nc(-c4cccc(-n5c6ccccc6c6c7cccc8c7n(c65)-c5ccccc5S8)c4)nc4c3sc3ccccc34)cc2)cc1]

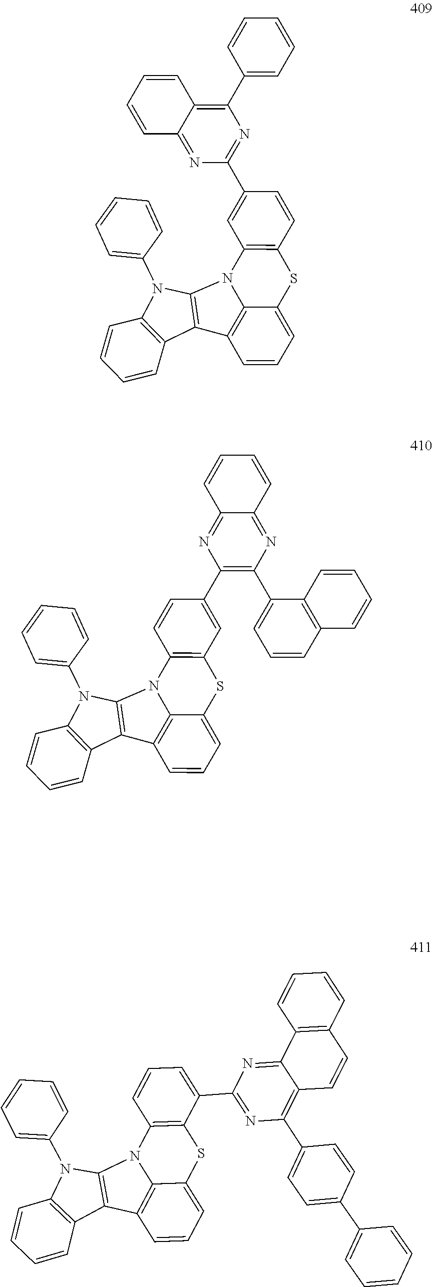 [SMILES: c1ccc(-c2nc(-c3ccc4c(c3)-n3c5c(cccc5c5c6ccccc6n(-c6ccccc6)c53)S4)nc3ccccc23)cc1 ; c1ccc(-n2c3ccccc3c3c4cccc5c4n(c32)-c2ccc(-c3nc4ccccc4nc3-c3cccc4ccccc34)cc2S5)cc1 ; c1ccc(-c2ccc(-c3nc(-c4cccc5c4Sc4cccc6c7c8ccccc8n(-c8ccccc8)c7n-5c46)nc4c3ccc3ccccc34)cc2)cc1]

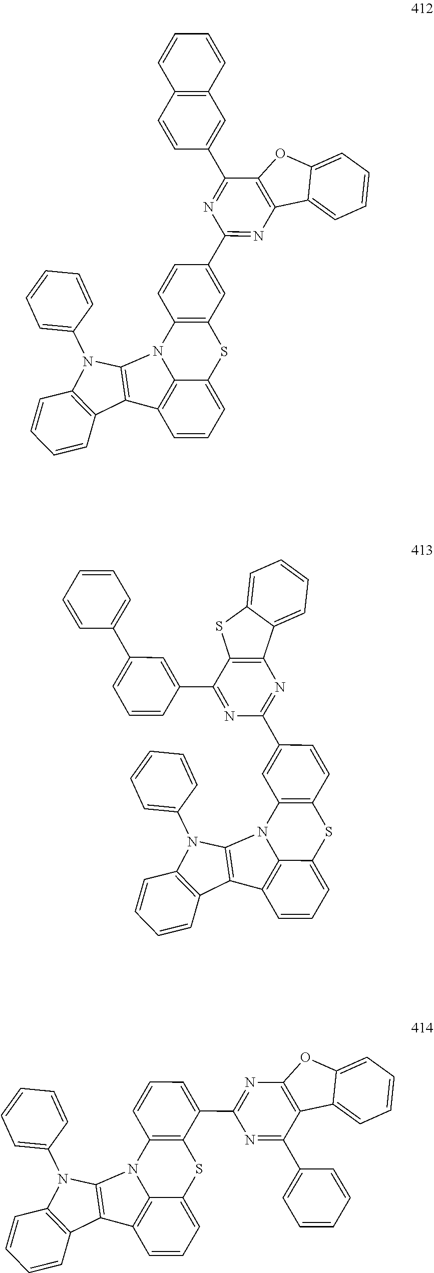 [SMILES: c1ccc(-n2c3ccccc3c3c4cccc5c4n(c32)-c2ccc(-c3nc(-c4ccc6ccccc6c4)c4oc6ccccc6c4n3)cc2S5)cc1 ; c1ccc(-c2cccc(-c3nc(-c4ccc5c(c4)-n4c6c(cccc6c6c7ccccc7n(-c7ccccc7)c64)S5)nc4c3sc3ccccc34)c2)cc1 ; c1ccc(-c2nc(-c3cccc4c3Sc3cccc5c6c7ccccc7n(-c7ccccc7)c6n-4c35)nc3oc4ccccc4c23)cc1]

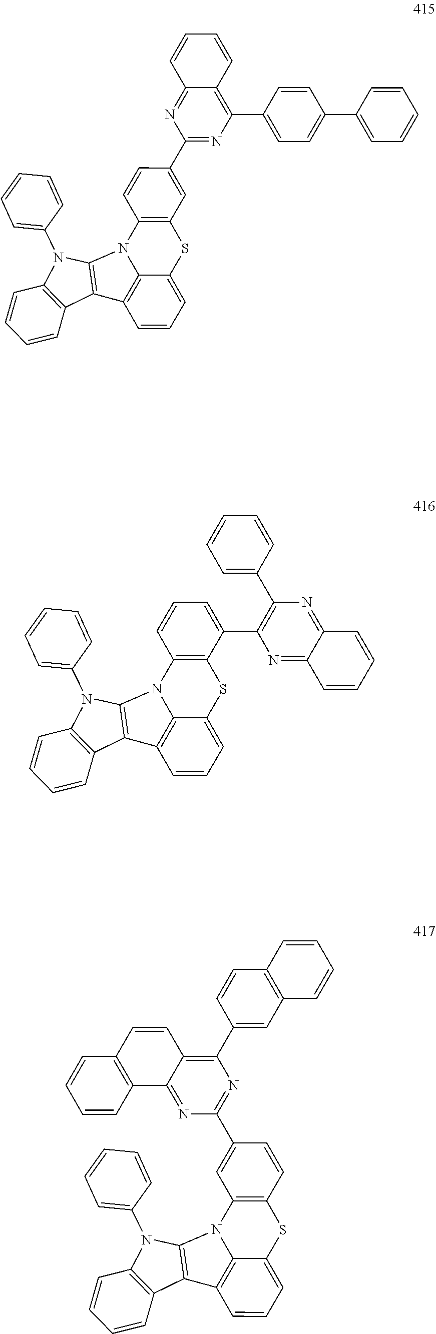 [SMILES: c1ccc(-c2ccc(-c3nc(-c4ccc5c(c4)Sc4cccc6c7c8ccccc8n(-c8ccccc8)c7n-5c46)nc4ccccc34)cc2)cc1 ; c1ccc(-c2nc3ccccc3nc2-c2cccc3c2Sc2cccc4c5c6ccccc6n(-c6ccccc6)c5n-3c24)cc1 ; c1ccc(-n2c3ccccc3c3c4cccc5c4n(c32)-c2cc(-c3nc(-c4ccc6ccccc6c4)c4ccc6ccccc6c4n3)ccc2S5)cc1]

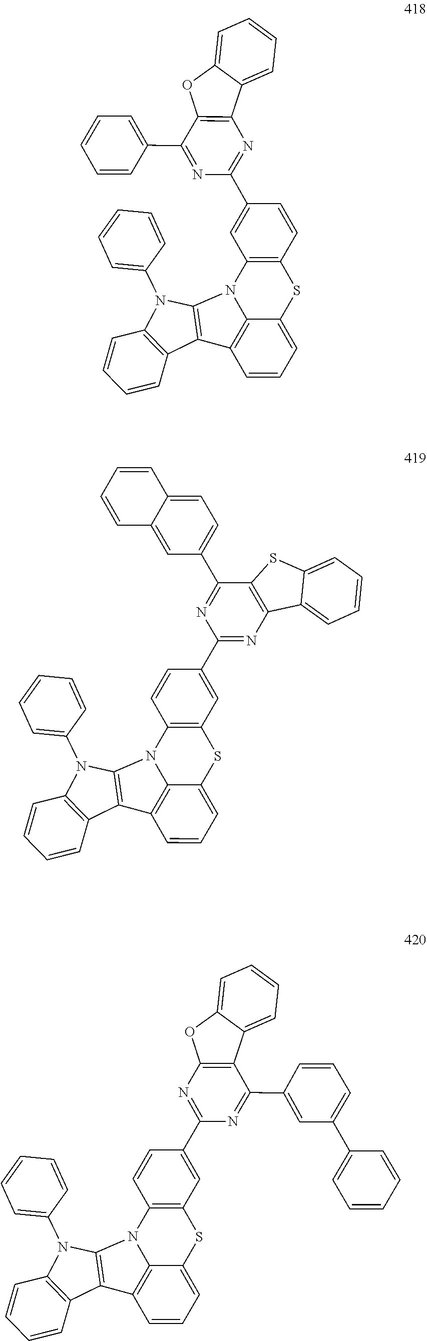 [SMILES: c1ccc(-c2nc(-c3ccc4c(c3)-n3c5c(cccc5c5c6ccccc6n(-c6ccccc6)c53)S4)nc3c2oc2ccccc23)cc1 ; c1ccc(-n2c3ccccc3c3c4cccc5c4n(c32)-c2ccc(-c3nc(-c4ccc6ccccc6c4)c4sc6ccccc6c4n3)cc2S5)cc1 ; c1ccc(-c2cccc(-c3nc(-c4ccc5c(c4)Sc4cccc6c7c8ccccc8n(-c8ccccc8)c7n-5c46)nc4oc5ccccc5c34)c2)cc1]

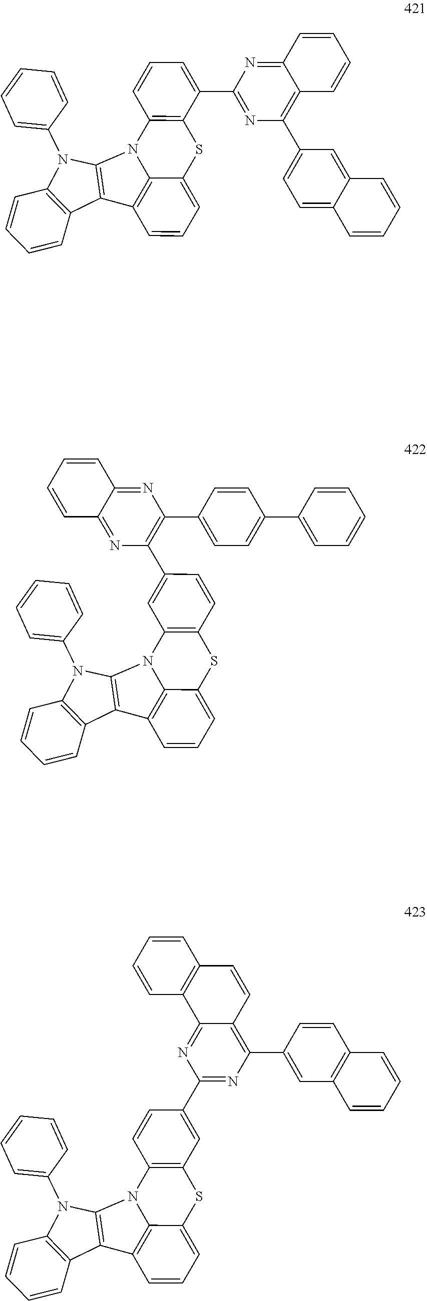 [SMILES: c1ccc(-n2c3ccccc3c3c4cccc5c4n(c32)-c2cccc(-c3nc(-c4ccc6ccccc6c4)c4ccccc4n3)c2S5)cc1 ; c1ccc(-c2ccc(-c3nc4ccccc4nc3-c3ccc4c(c3)-n3c5c(cccc5c5c6ccccc6n(-c6ccccc6)c53)S4)cc2)cc1 ; c1ccc(-n2c3ccccc3c3c4cccc5c4n(c32)-c2ccc(-c3nc(-c4ccc6ccccc6c4)c4ccc6ccccc6c4n3)cc2S5)cc1]

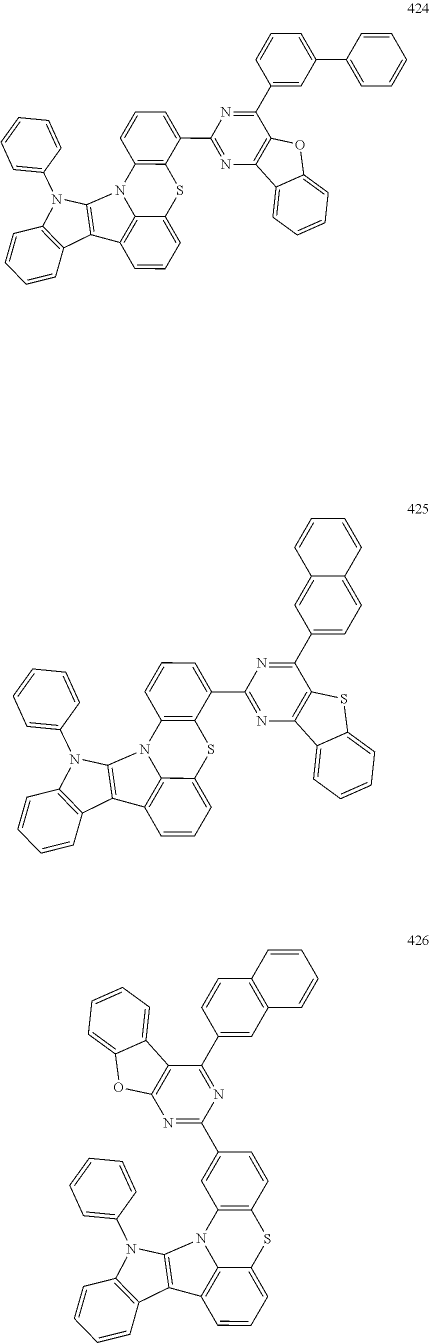 [SMILES: c1ccc(-c2cccc(-c3nc(-c4cccc5c4Sc4cccc6c7c8ccccc8n(-c8ccccc8)c7n-5c46)nc4c3oc3ccccc34)c2)cc1 ; c1ccc(-n2c3ccccc3c3c4cccc5c4n(c32)-c2cccc(-c3nc(-c4ccc6ccccc6c4)c4sc6ccccc6c4n3)c2S5)cc1 ; c1ccc(-n2c3ccccc3c3c4cccc5c4n(c32)-c2cc(-c3nc(-c4ccc6ccccc6c4)c4c(n3)oc3ccccc34)ccc2S5)cc1]

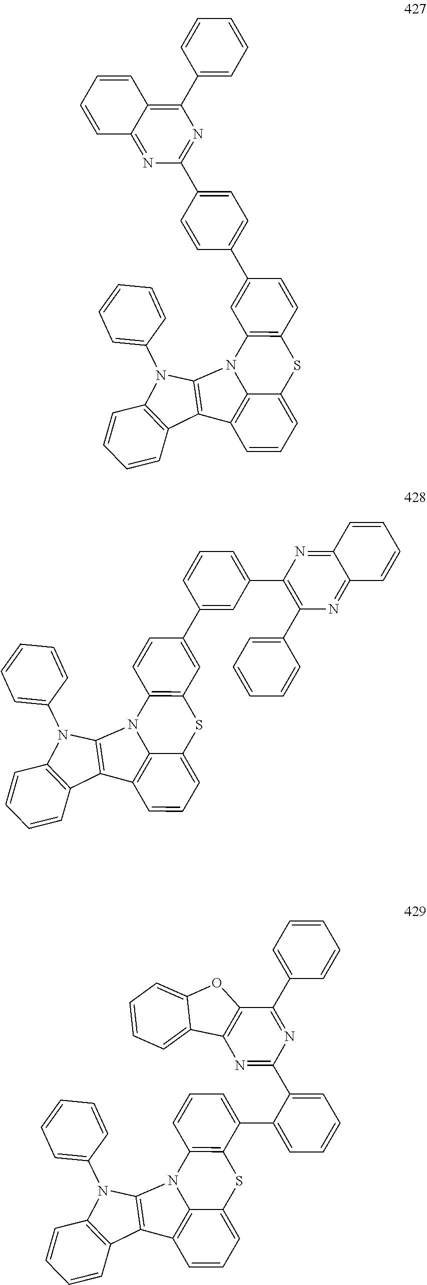 [SMILES: c1ccc(-c2nc(-c3ccc(-c4ccc5c(c4)-n4c6c(cccc6c6c7ccccc7n(-c7ccccc7)c64)S5)cc3)nc3ccccc23)cc1 ; c1ccc(-c2nc3ccccc3nc2-c2cccc(-c3ccc4c(c3)Sc3cccc5c6c7ccccc7n(-c7ccccc7)c6n-4c35)c2)cc1 ; c1ccc(-c2nc(-c3ccccc3-c3cccc4c3Sc3cccc5c6c7ccccc7n(-c7ccccc7)c6n-4c35)nc3c2oc2ccccc23)cc1]

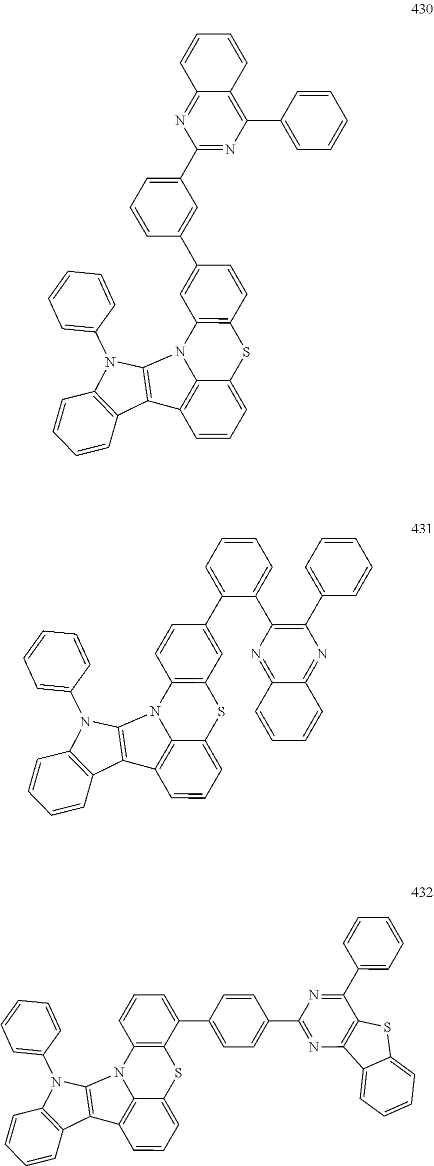 [SMILES: c1ccc(-c2nc(-c3cccc(-c4ccc5c(c4)-n4c6c(cccc6c6c7ccccc7n(-c7ccccc7)c64)S5)c3)nc3ccccc23)cc1 ; c1ccc(-c2nc3ccccc3nc2-c2ccccc2-c2ccc3c(c2)Sc2cccc4c5c6ccccc6n(-c6ccccc6)c5n-3c24)cc1 ; c1ccc(-c2nc(-c3ccc(-c4cccc5c4Sc4cccc6c7c8ccccc8n(-c8ccccc8)c7n-5c46)cc3)nc3c2sc2ccccc23)cc1]

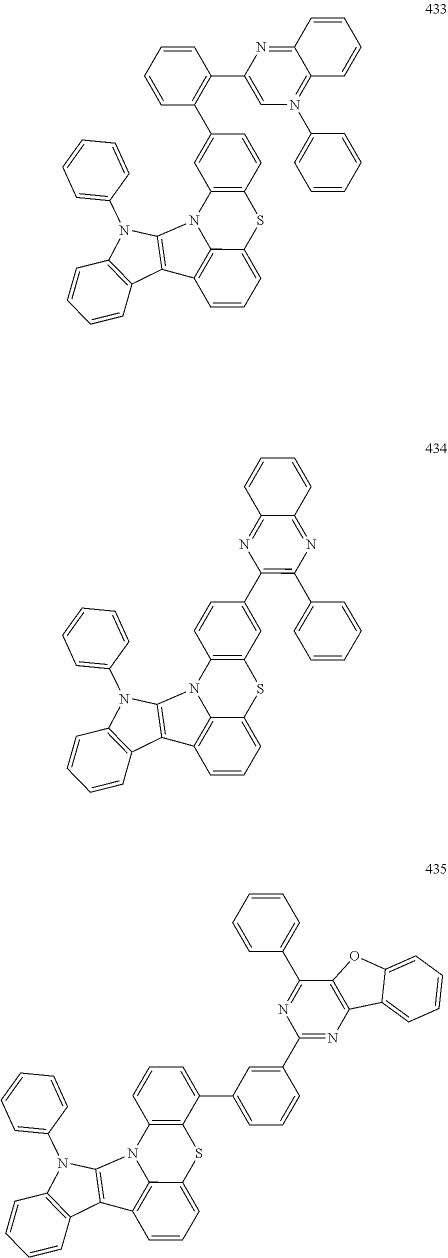

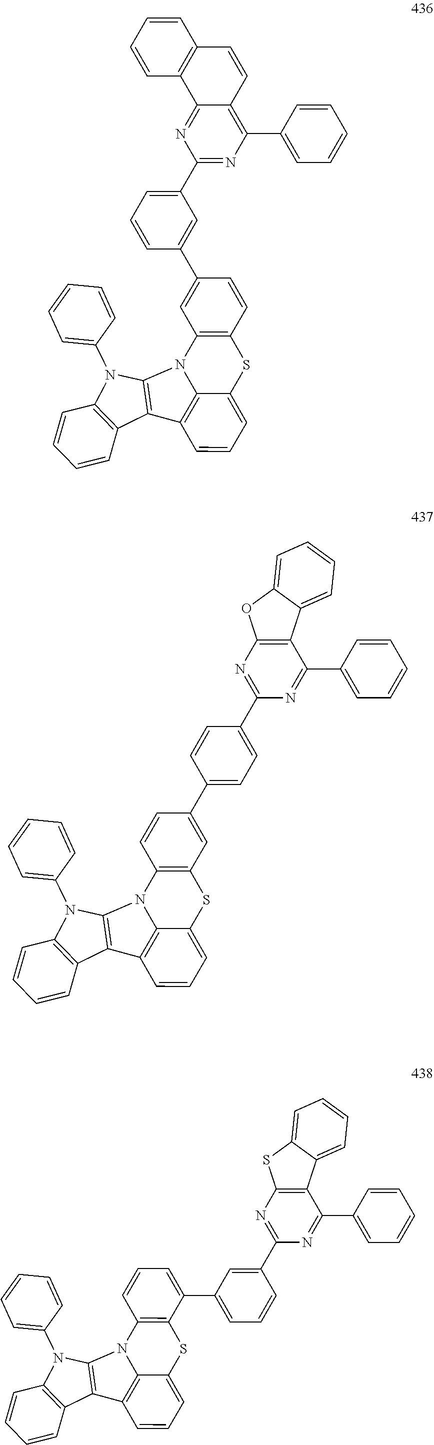 [SMILES: c1ccc(-c2nc(-c3cccc(-c4ccc5c(c4)-n4c6c(cccc6c6c7ccccc7n(-c7ccccc7)c64)S5)c3)nc3c2ccc2ccccc23)cc1 ; c1ccc(-c2nc(-c3ccc(-c4ccc5c(c4)Sc4cccc6c7c8ccccc8n(-c8ccccc8)c7n-5c46)cc3)nc3oc4ccccc4c23)cc1 ; c1ccc(-c2nc(-c3cccc(-c4cccc5c4Sc4cccc6c7c8ccccc8n(-c8ccccc8)c7n-5c46)c3)nc3sc4ccccc4c23)cc1]

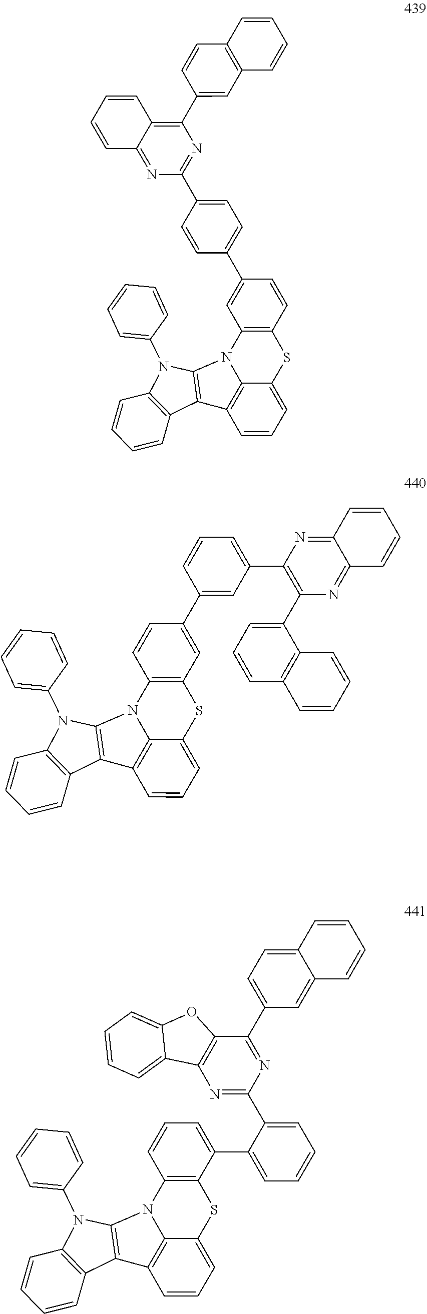 [SMILES: c1ccc(-n2c3ccccc3c3c4cccc5c4n(c32)-c2cc(-c3ccc(-c4nc(-c6ccc7ccccc7c6)c6ccccc6n4)cc3)ccc2S5)cc1 ; c1ccc(-n2c3ccccc3c3c4cccc5c4n(c32)-c2ccc(-c3cccc(-c4nc6ccccc6nc4-c4cccc6ccccc46)c3)cc2S5)cc1 ; c1ccc(-n2c3ccccc3c3c4cccc5c4n(c32)-c2cccc(-c3ccccc3-c3nc(-c4ccc6ccccc6c4)c4oc6ccccc6c4n3)c2S5)cc1]

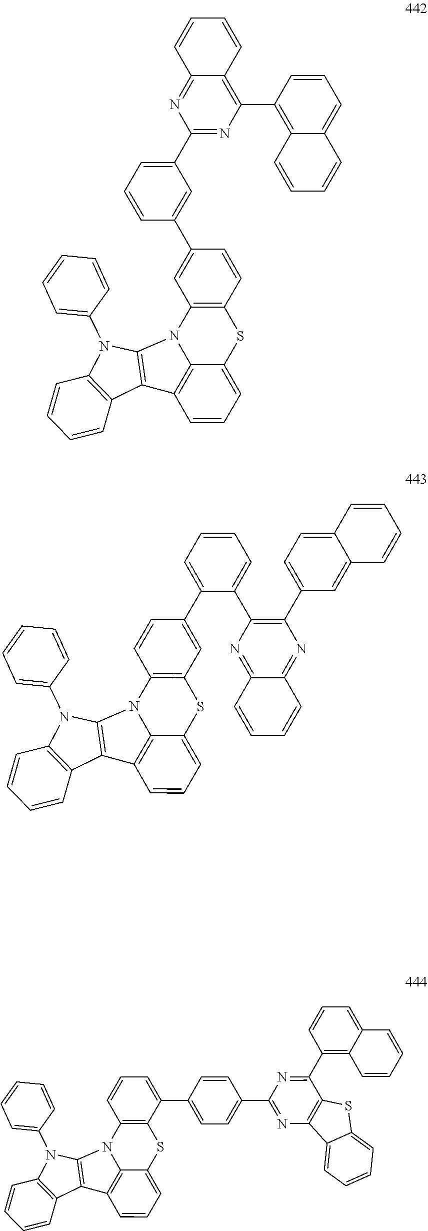 [SMILES: c1ccc(-n2c3ccccc3c3c4cccc5c4n(c32)-c2cc(-c3cccc(-c4nc(-c6cccc7ccccc67)c6ccccc6n4)c3)ccc2S5)cc1 ; c1ccc(-n2c3ccccc3c3c4cccc5c4n(c32)-c2ccc(-c3ccccc3-c3nc4ccccc4nc3-c3ccc4ccccc4c3)cc2S5)cc1 ; c1ccc(-n2c3ccccc3c3c4cccc5c4n(c32)-c2cccc(-c3ccc(-c4nc(-c6cccc7ccccc67)c6sc7ccccc7c6n4)cc3)c2S5)cc1]

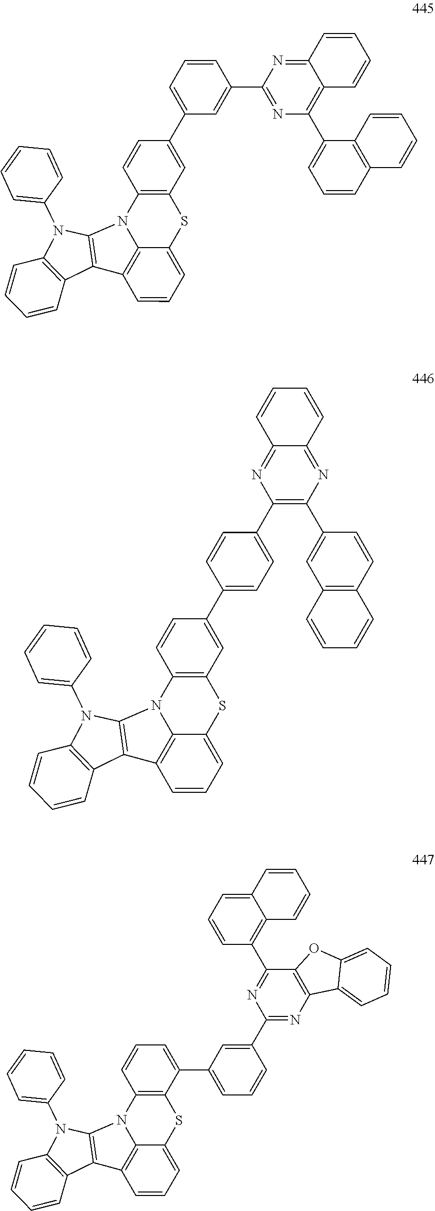 [SMILES: c1ccc(-n2c3ccccc3c3c4cccc5c4n(c32)-c2ccc(-c3cccc(-c4nc(-c6cccc7ccccc67)c6ccccc6n4)c3)cc2S5)cc1 ; c1ccc(-n2c3ccccc3c3c4cccc5c4n(c32)-c2ccc(-c3ccc(-c4nc6ccccc6nc4-c4ccc6ccccc6c4)cc3)cc2S5)cc1 ; c1ccc(-n2c3ccccc3c3c4cccc5c4n(c32)-c2cccc(-c3cccc(-c4nc(-c6cccc7ccccc67)c6oc7ccccc7c6n4)c3)c2S5)cc1]

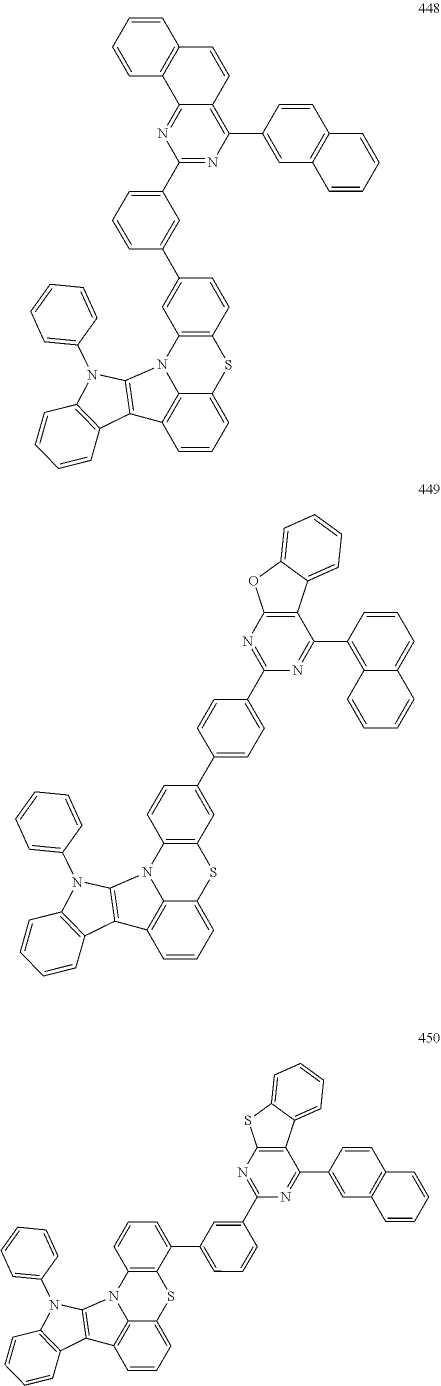 [SMILES: c1ccc(-n2c3ccccc3c3c4cccc5c4n(c32)-c2cc(-c3cccc(-c4nc(-c6ccc7ccccc7c6)c6ccc7ccccc7c6n4)c3)ccc2S5)cc1 ; c1ccc(-n2c3ccccc3c3c4cccc5c4n(c32)-c2ccc(-c3ccc(-c4nc(-c6cccc7ccccc67)c6c(n4)oc4ccccc46)cc3)cc2S5)cc1 ; c1ccc(-n2c3ccccc3c3c4cccc5c4n(c32)-c2cccc(-c3cccc(-c4nc(-c6ccc7ccccc7c6)c6c(n4)sc4ccccc46)c3)c2S5)cc1]

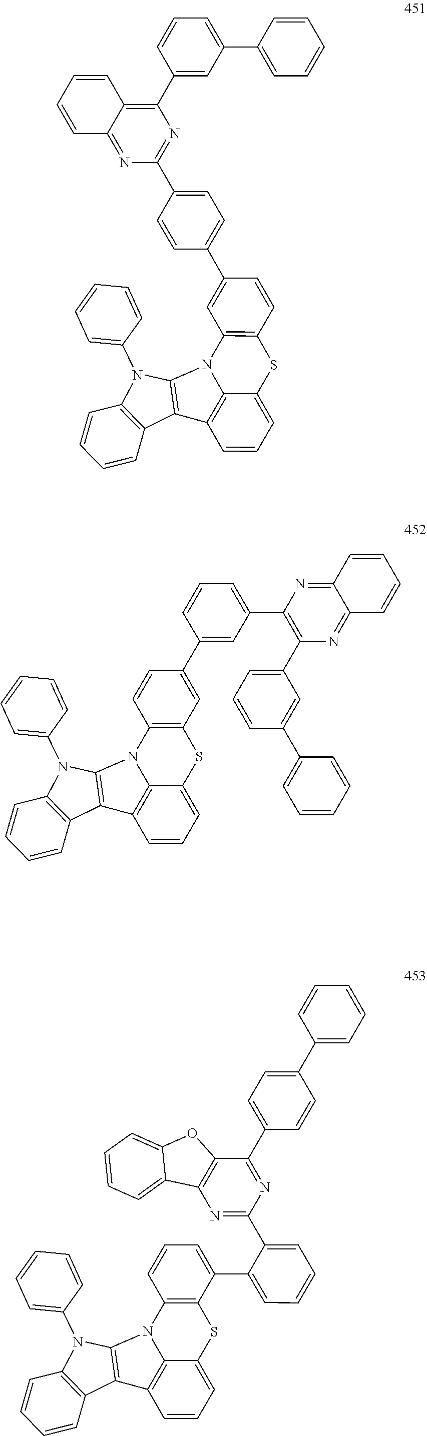 [SMILES: c1ccc(-c2cccc(-c3nc(-c4ccc(-c5ccc6c(c5)-n5c7c(cccc7c7c8ccccc8n(-c8ccccc8)c75)S6)cc4)nc4ccccc34)c2)cc1 ; c1ccc(-c2cccc(-c3nc4ccccc4nc3-c3cccc(-c4ccc5c(c4)Sc4cccc6c7c8ccccc8n(-c8ccccc8)c7n-5c46)c3)c2)cc1 ; c1ccc(-c2ccc(-c3nc(-c4ccccc4-c4cccc5c4Sc4cccc6c7c8ccccc8n(-c8ccccc8)c7n-5c46)nc4c3oc3ccccc34)cc2)cc1]

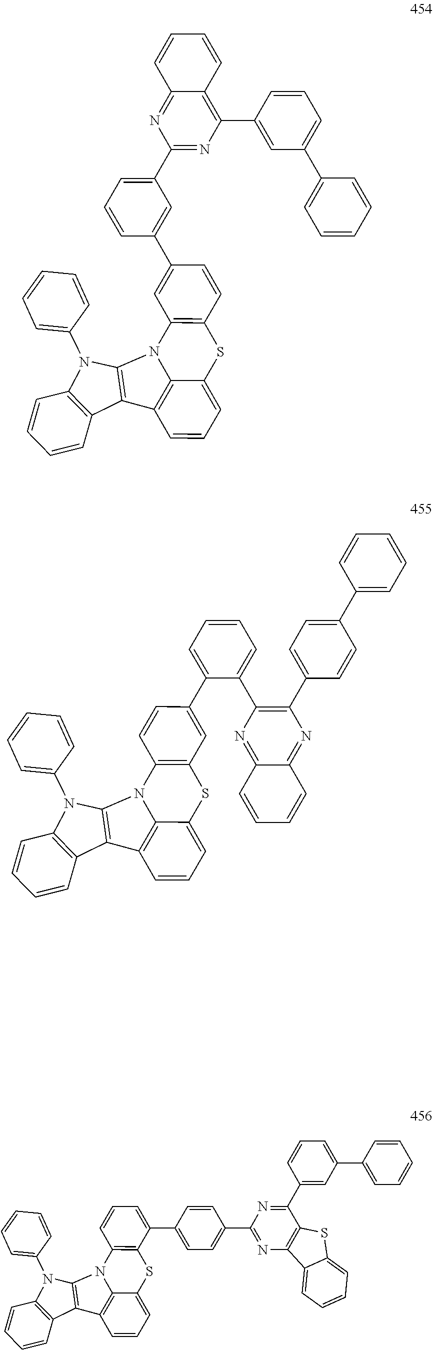 [SMILES: c1ccc(-c2cccc(-c3nc(-c4cccc(-c5ccc6c(c5)-n5c7c(cccc7c7c8ccccc8n(-c8ccccc8)c75)S6)c4)nc4ccccc34)c2)cc1 ; c1ccc(-c2ccc(-c3nc4ccccc4nc3-c3ccccc3-c3ccc4c(c3)Sc3cccc5c6c7ccccc7n(-c7ccccc7)c6n-4c35)cc2)cc1 ; c1ccc(-c2cccc(-c3nc(-c4ccc(-c5cccc6c5Sc5cccc7c8c9ccccc9n(-c9ccccc9)c8n-6c57)cc4)nc4c3sc3ccccc34)c2)cc1]

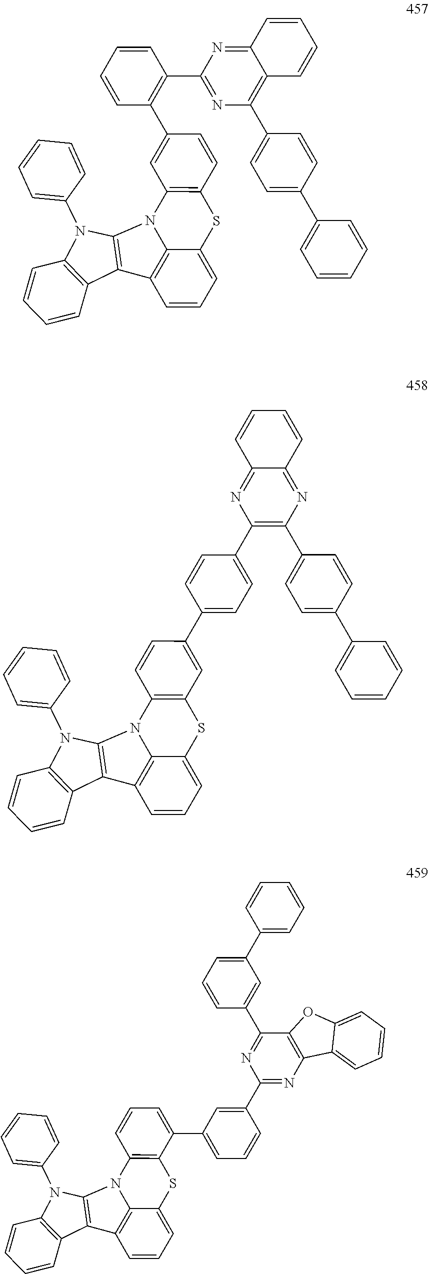 [SMILES: c1ccc(-c2ccc(-c3nc(-c4ccccc4-c4ccc5c(c4)-n4c6c(cccc6c6c7ccccc7n(-c7ccccc7)c64)S5)nc4ccccc34)cc2)cc1 ; c1ccc(-c2ccc(-c3nc4ccccc4nc3-c3ccc(-c4ccc5c(c4)Sc4cccc6c7c8ccccc8n(-c8ccccc8)c7n-5c46)cc3)cc2)cc1 ; c1ccc(-c2cccc(-c3nc(-c4cccc(-c5cccc6c5Sc5cccc7c8c9ccccc9n(-c9ccccc9)c8n-6c57)c4)nc4c3oc3ccccc34)c2)cc1]

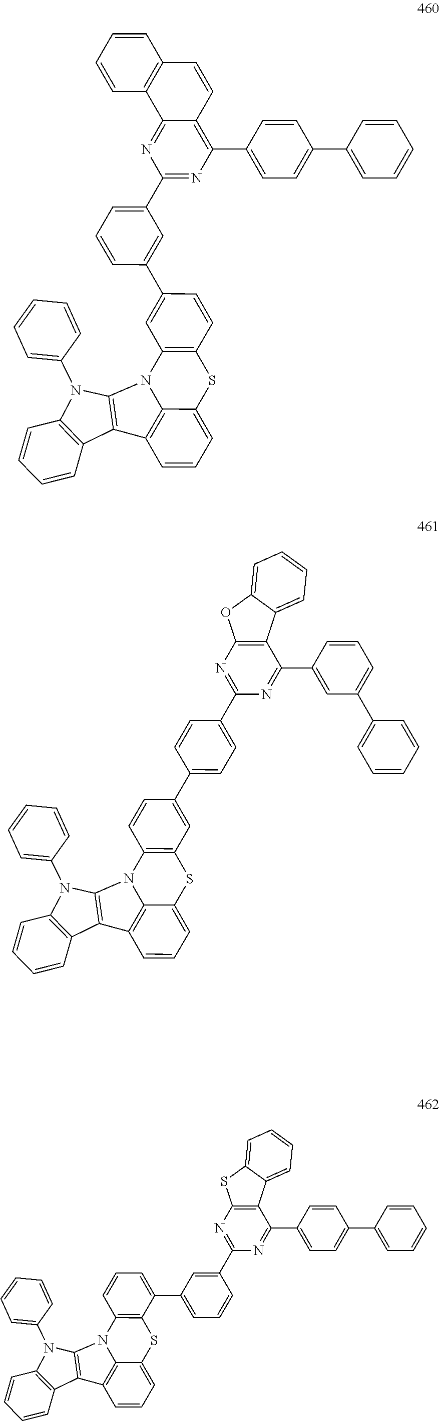 [SMILES: c1ccc(-c2ccc(-c3nc(-c4cccc(-c5ccc6c(c5)-n5c7c(cccc7c7c8ccccc8n(-c8ccccc8)c75)S6)c4)nc4c3ccc3ccccc34)cc2)cc1 ; c1ccc(-c2cccc(-c3nc(-c4ccc(-c5ccc6c(c5)Sc5cccc7c8c9ccccc9n(-c9ccccc9)c8n-6c57)cc4)nc4oc5ccccc5c34)c2)cc1 ; c1ccc(-c2ccc(-c3nc(-c4cccc(-c5cccc6c5Sc5cccc7c8c9ccccc9n(-c9ccccc9)c8n-6c57)c4)nc4sc5ccccc5c34)cc2)cc1]

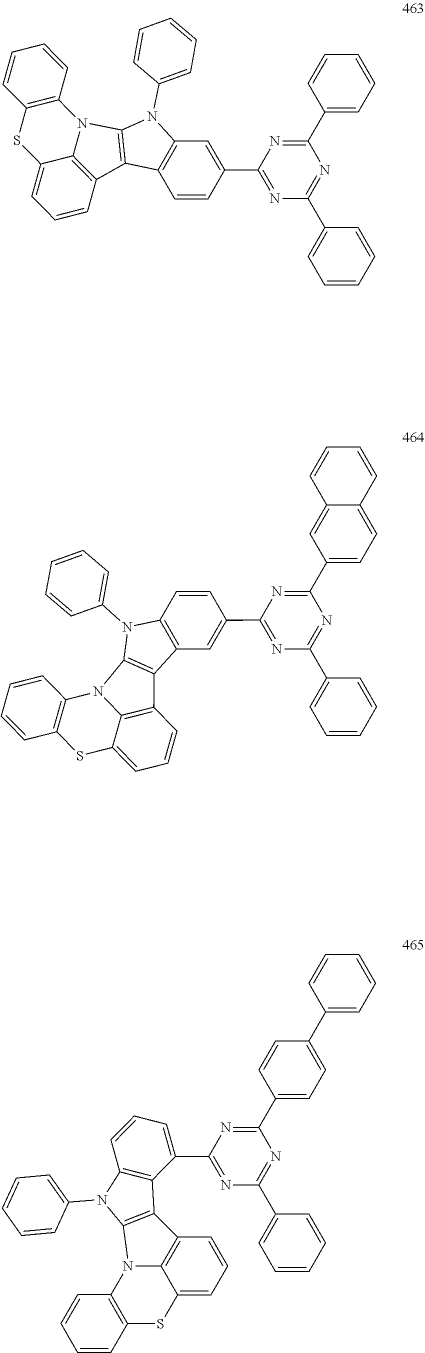 [SMILES: c1ccc(-c2nc(-c3ccccc3)nc(-c3ccc4c5c6cccc7c6n(c5n(-c5ccccc5)c4c3)-c3ccccc3S7)n2)cc1 ; c1ccc(-c2nc(-c3ccc4ccccc4c3)nc(-c3ccc4c(c3)c3c5cccc6c5n(c3n4-c3ccccc3)-c3ccccc3S6)n2)cc1 ; c1ccc(-c2ccc(-c3nc(-c4ccccc4)nc(-c4cccc5c4c4c6cccc7c6n(c4n5-c4ccccc4)-c4ccccc4S7)n3)cc2)cc1]

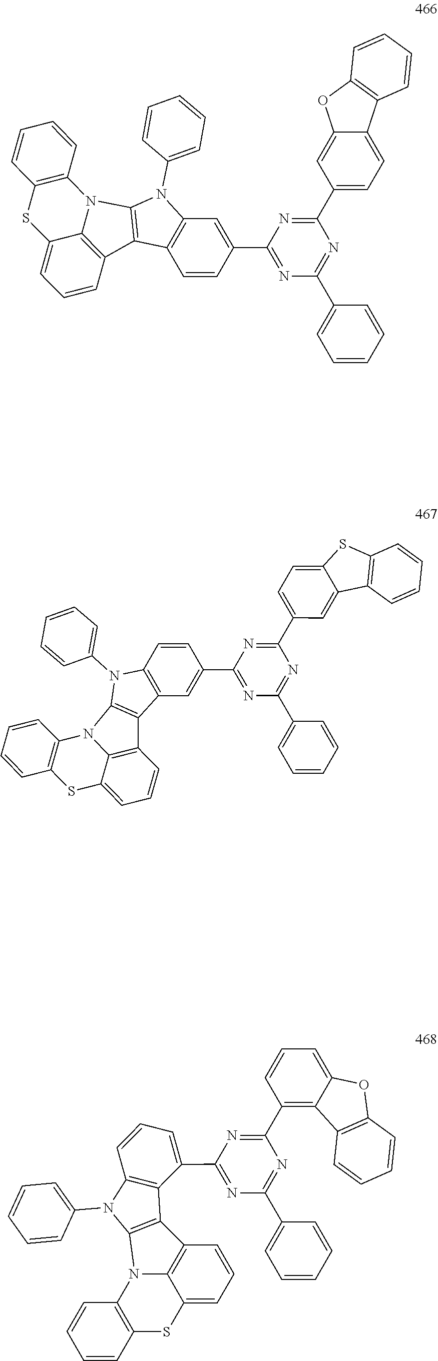 [SMILES: c1ccc(-c2nc(-c3ccc4c(c3)oc3ccccc34)nc(-c3ccc4c5c6cccc7c6n(c5n(-c5ccccc5)c4c3)-c3ccccc3S7)n2)cc1 ; c1ccc(-c2nc(-c3ccc4sc5ccccc5c4c3)nc(-c3ccc4c(c3)c3c5cccc6c5n(c3n4-c3ccccc3)-c3ccccc3S6)n2)cc1 ; c1ccc(-c2nc(-c3cccc4oc5ccccc5c34)nc(-c3cccc4c3c3c5cccc6c5n(c3n4-c3ccccc3)-c3ccccc3S6)n2)cc1]

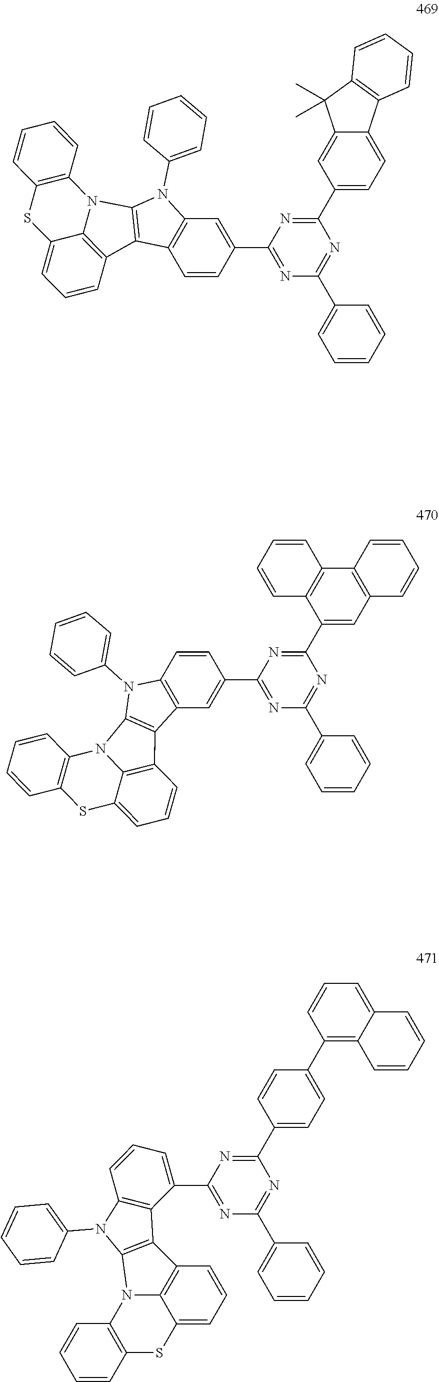 [SMILES: CC1(C)c2ccccc2-c2ccc(-c3nc(-c4ccccc4)nc(-c4ccc5c6c7cccc8c7n(c6n(-c6ccccc6)c5c4)-c4ccccc4S8)n3)cc21 ; c1ccc(-c2nc(-c3ccc4c(c3)c3c5cccc6c5n(c3n4-c3ccccc3)-c3ccccc3S6)nc(-c3cc4ccccc4c4ccccc34)n2)cc1 ; c1ccc(-c2nc(-c3ccc(-c4cccc5ccccc45)cc3)nc(-c3cccc4c3c3c5cccc6c5n(c3n4-c3ccccc3)-c3ccccc3S6)n2)cc1]

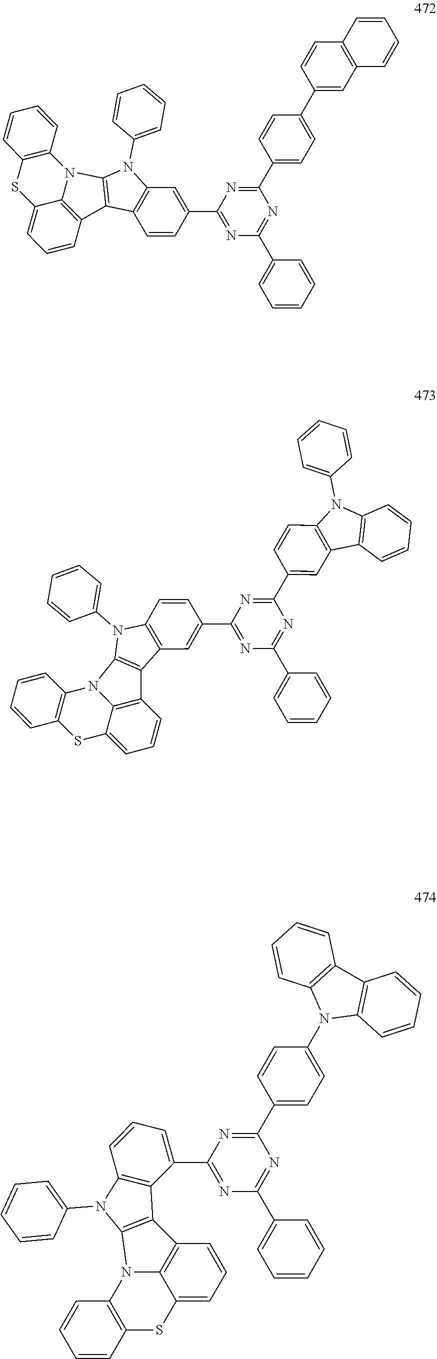 [SMILES: c1ccc(-c2nc(-c3ccc(-c4ccc5ccccc5c4)cc3)nc(-c3ccc4c5c6cccc7c6n(c5n(-c5ccccc5)c4c3)-c3ccccc3S7)n2)cc1 ; c1ccc(-c2nc(-c3ccc4c(c3)c3ccccc3n4-c3ccccc3)nc(-c3ccc4c(c3)c3c5cccc6c5n(c3n4-c3ccccc3)-c3ccccc3S6)n2)cc1 ; c1ccc(-c2nc(-c3ccc(-n4c5ccccc5c5ccccc54)cc3)nc(-c3cccc4c3c3c5cccc6c5n(c3n4-c3ccccc3)-c3ccccc3S6)n2)cc1]

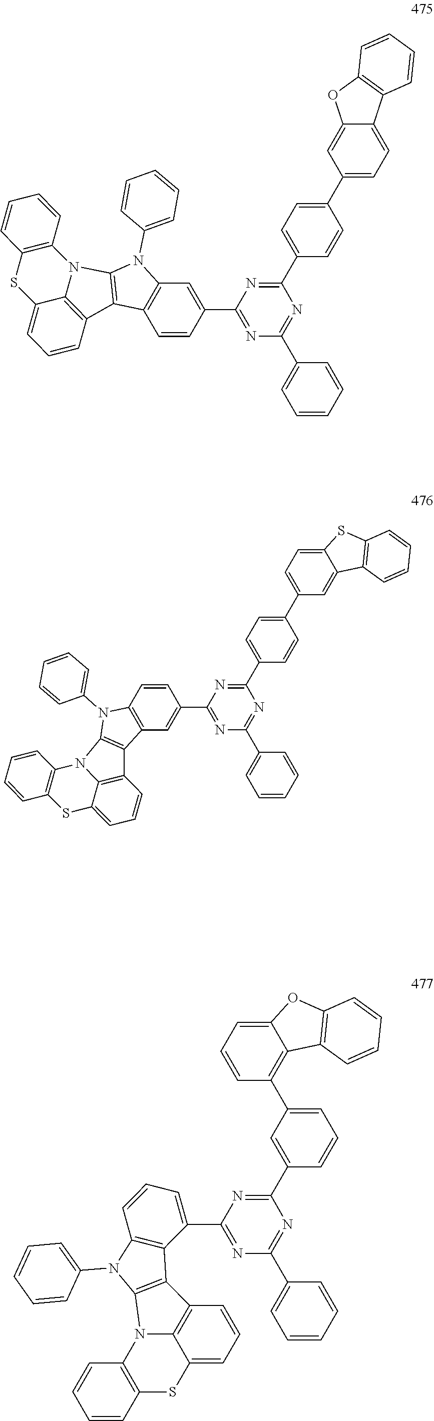 [SMILES: c1ccc(-c2nc(-c3ccc(-c4ccc5c(c4)oc4ccccc45)cc3)nc(-c3ccc4c5c6cccc7c6n(c5n(-c5ccccc5)c4c3)-c3ccccc3S7)n2)cc1 ; c1ccc(-c2nc(-c3ccc(-c4ccc5sc6ccccc6c5c4)cc3)nc(-c3ccc4c(c3)c3c5cccc6c5n(c3n4-c3ccccc3)-c3ccccc3S6)n2)cc1 ; c1ccc(-c2nc(-c3cccc(-c4cccc5oc6ccccc6c45)c3)nc(-c3cccc4c3c3c5cccc6c5n(c3n4-c3ccccc3)-c3ccccc3S6)n2)cc1]

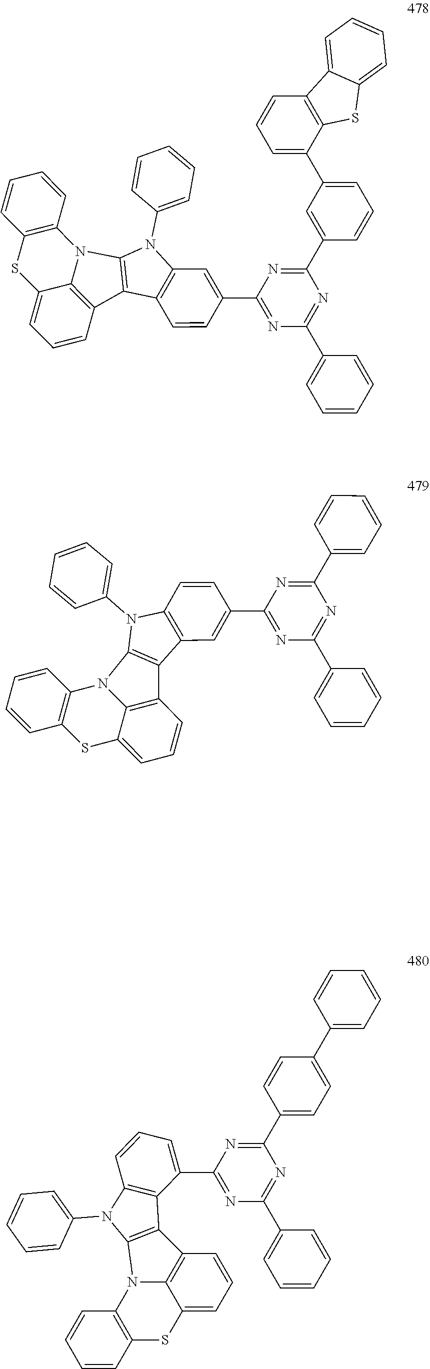 [SMILES: c1ccc(-c2nc(-c3cccc(-c4cccc5c4sc4ccccc45)c3)nc(-c3ccc4c5c6cccc7c6n(c5n(-c5ccccc5)c4c3)-c3ccccc3S7)n2)cc1 ; c1ccc(-c2nc(-c3ccccc3)nc(-c3ccc4c(c3)c3c5cccc6c5n(c3n4-c3ccccc3)-c3ccccc3S6)n2)cc1 ; c1ccc(-c2ccc(-c3nc(-c4ccccc4)nc(-c4cccc5c4c4c6cccc7c6n(c4n5-c4ccccc4)-c4ccccc4S7)n3)cc2)cc1]

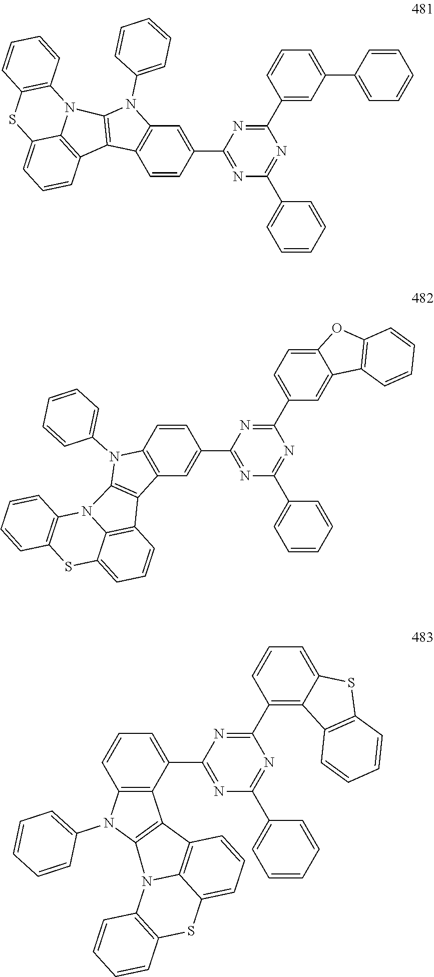 [SMILES: c1ccc(-c2cccc(-c3nc(-c4ccccc4)nc(-c4ccc5c6c7cccc8c7n(c6n(-c6ccccc6)c5c4)-c4ccccc4S8)n3)c2)cc1 ; c1ccc(-c2nc(-c3ccc4oc5ccccc5c4c3)nc(-c3ccc4c(c3)c3c5cccc6c5n(c3n4-c3ccccc3)-c3ccccc3S6)n2)cc1 ; c1ccc(-c2nc(-c3cccc4sc5ccccc5c34)nc(-c3cccc4c3c3c5cccc6c5n(c3n4-c3ccccc3)-c3ccccc3S6)n2)cc1]

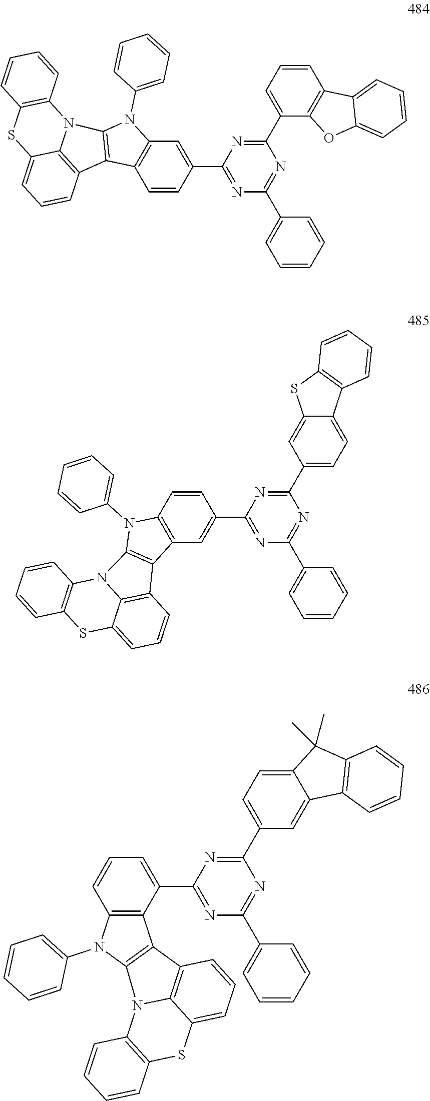 [SMILES: c1ccc(-c2nc(-c3ccc4c5c6cccc7c6n(c5n(-c5ccccc5)c4c3)-c3ccccc3S7)nc(-c3cccc4c3oc3ccccc34)n2)cc1 ; c1ccc(-c2nc(-c3ccc4c(c3)sc3ccccc34)nc(-c3ccc4c(c3)c3c5cccc6c5n(c3n4-c3ccccc3)-c3ccccc3S6)n2)cc1 ; CC1(C)c2ccccc2-c2cc(-c3nc(-c4ccccc4)nc(-c4cccc5c4c4c6cccc7c6n(c4n5-c4ccccc4)-c4ccccc4S7)n3)ccc21]

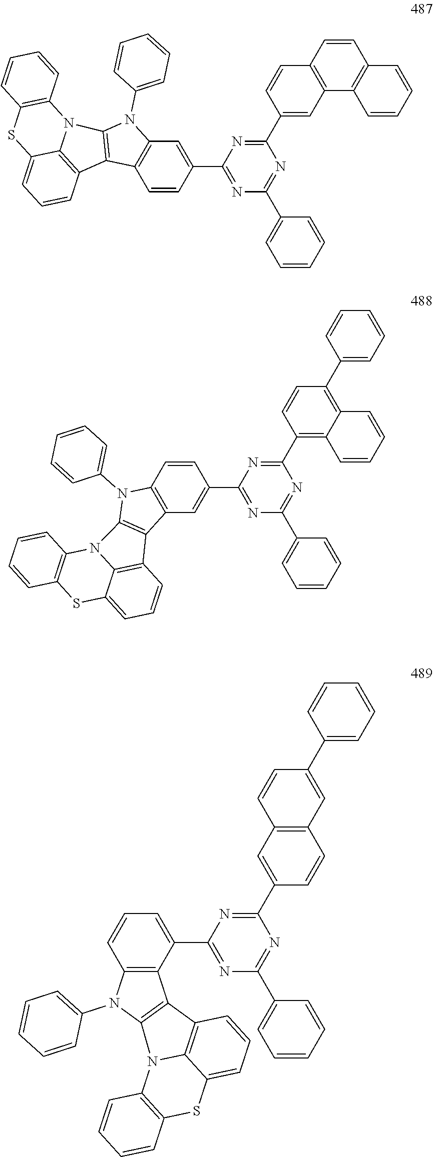 [SMILES: c1ccc(-c2nc(-c3ccc4ccc5ccccc5c4c3)nc(-c3ccc4c5c6cccc7c6n(c5n(-c5ccccc5)c4c3)-c3ccccc3S7)n2)cc1 ; c1ccc(-c2nc(-c3ccc4c(c3)c3c5cccc6c5n(c3n4-c3ccccc3)-c3ccccc3S6)nc(-c3ccc(-c4ccccc4)c4ccccc34)n2)cc1 ; c1ccc(-c2ccc3cc(-c4nc(-c5ccccc5)nc(-c5cccc6c5c5c7cccc8c7n(c5n6-c5ccccc5)-c5ccccc5S8)n4)ccc3c2)cc1]

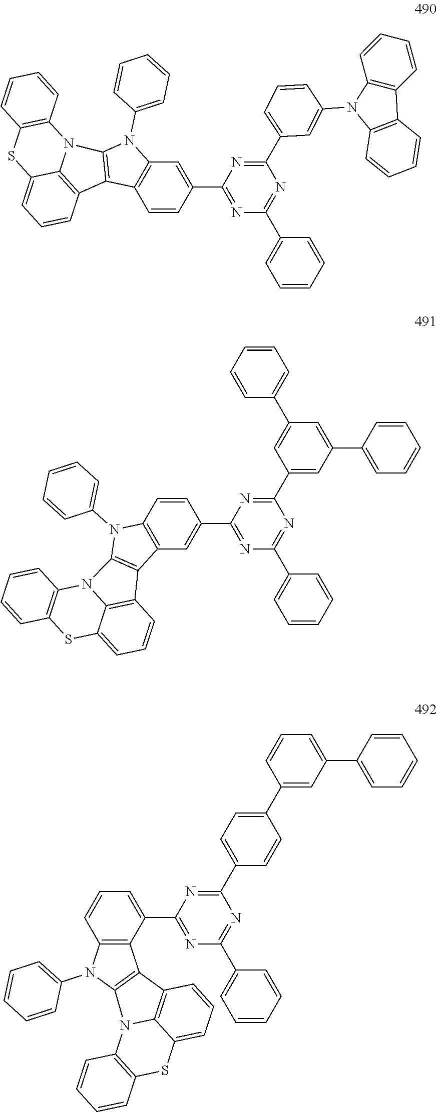 [SMILES: c1ccc(-c2nc(-c3cccc(-n4c5ccccc5c5ccccc54)c3)nc(-c3ccc4c5c6cccc7c6n(c5n(-c5ccccc5)c4c3)-c3ccccc3S7)n2)cc1 ; c1ccc(-c2cc(-c3ccccc3)cc(-c3nc(-c4ccccc4)nc(-c4ccc5c(c4)c4c6cccc7c6n(c4n5-c4ccccc4)-c4ccccc4S7)n3)c2)cc1 ; c1ccc(-c2cccc(-c3ccc(-c4nc(-c5ccccc5)nc(-c5cccc6c5c5c7cccc8c7n(c5n6-c5ccccc5)-c5ccccc5S8)n4)cc3)c2)cc1]

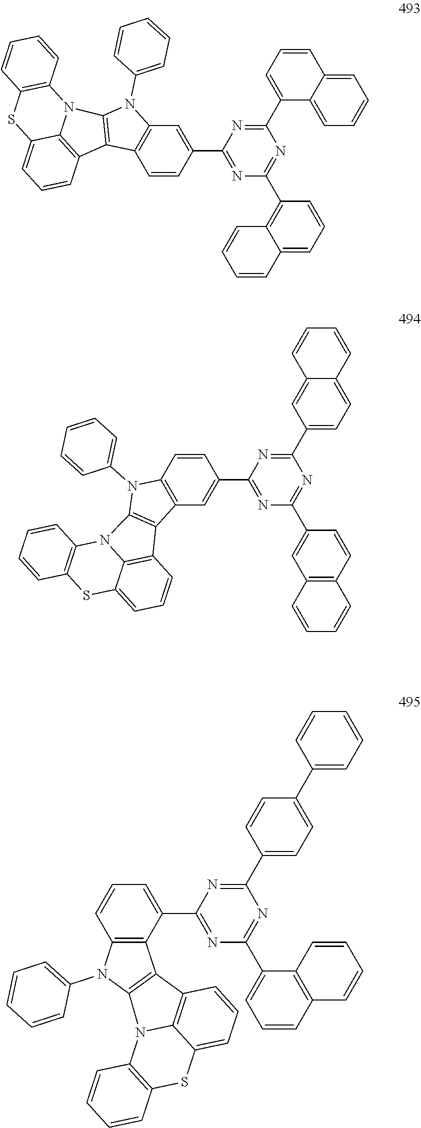 [SMILES: c1ccc(-n2c3cc(-c4nc(-c5cccc6ccccc56)nc(-c5cccc6ccccc56)n4)ccc3c3c4cccc5c4n(c32)-c2ccccc2S5)cc1 ; c1ccc(-n2c3ccc(-c4nc(-c5ccc6ccccc6c5)nc(-c5ccc6ccccc6c5)n4)cc3c3c4cccc5c4n(c32)-c2ccccc2S5)cc1 ; c1ccc(-c2ccc(-c3nc(-c4cccc5ccccc45)nc(-c4cccc5c4c4c6cccc7c6n(c4n5-c4ccccc4)-c4ccccc4S7)n3)cc2)cc1]

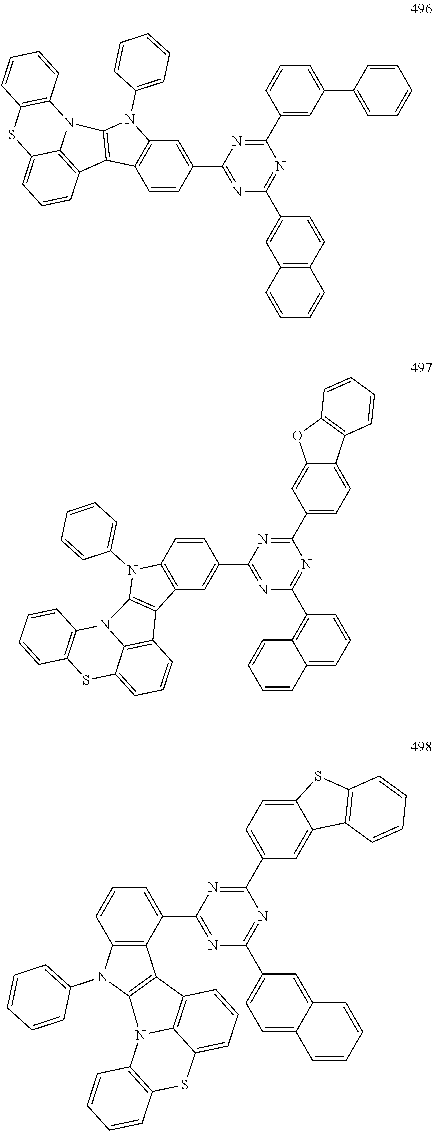 [SMILES: c1ccc(-c2cccc(-c3nc(-c4ccc5ccccc5c4)nc(-c4ccc5c6c7cccc8c7n(c6n(-c6ccccc6)c5c4)-c4ccccc4S8)n3)c2)cc1 ; c1ccc(-n2c3ccc(-c4nc(-c5ccc6c(c5)oc5ccccc56)nc(-c5cccc6ccccc56)n4)cc3c3c4cccc5c4n(c32)-c2ccccc2S5)cc1 ; c1ccc(-n2c3cccc(-c4nc(-c5ccc6ccccc6c5)nc(-c5ccc6sc7ccccc7c6c5)n4)c3c3c4cccc5c4n(c32)-c2ccccc2S5)cc1]

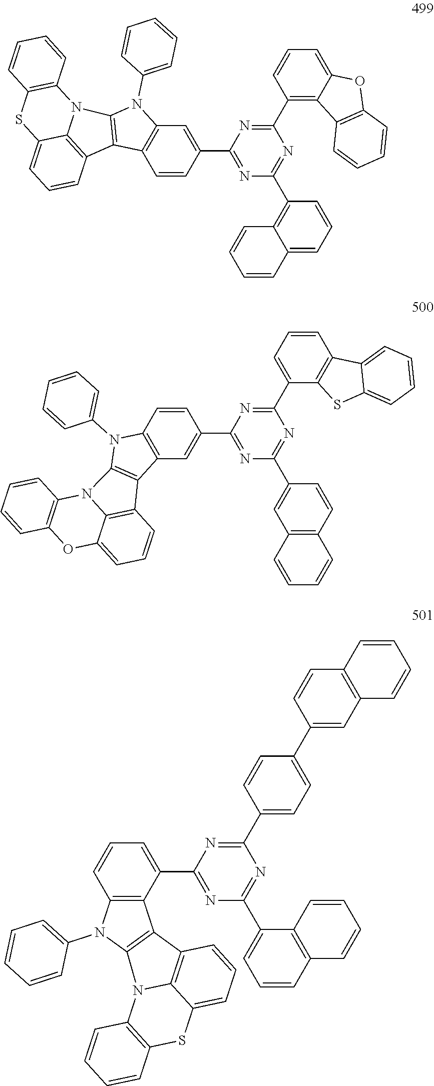 [SMILES: c1ccc(-n2c3cc(-c4nc(-c5cccc6ccccc56)nc(-c5cccc6oc7ccccc7c56)n4)ccc3c3c4cccc5c4n(c32)-c2ccccc2S5)cc1 ; c1ccc(-n2c3ccc(-c4nc(-c5ccc6ccccc6c5)nc(-c5cccc6c5sc5ccccc56)n4)cc3c3c4cccc5c4n(c32)-c2ccccc2O5)cc1 ; c1ccc(-n2c3cccc(-c4nc(-c5ccc(-c6ccc7ccccc7c6)cc5)nc(-c5cccc6ccccc56)n4)c3c3c4cccc5c4n(c32)-c2ccccc2S5)cc1]

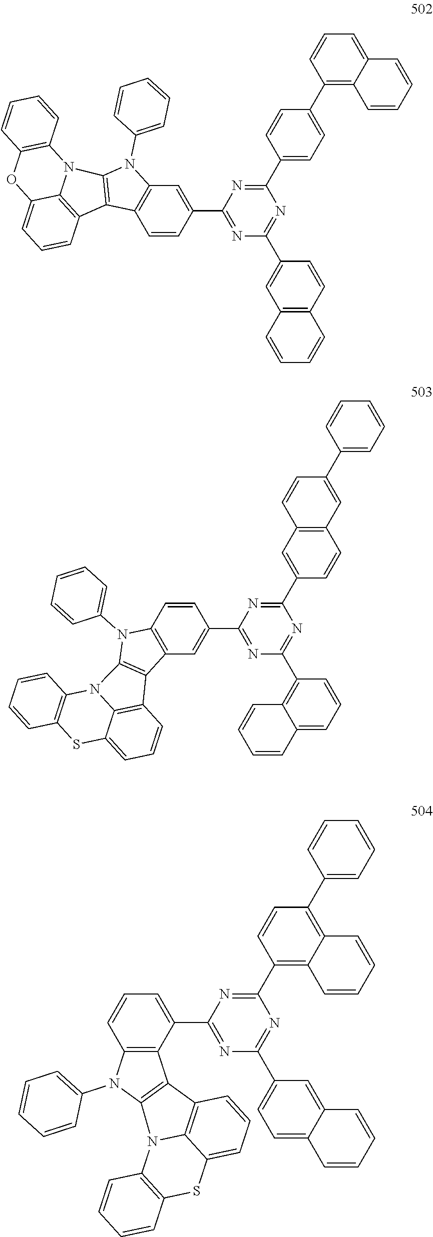 [SMILES: c1ccc(-n2c3cc(-c4nc(-c5ccc(-c6cccc7ccccc67)cc5)nc(-c5ccc6ccccc6c5)n4)ccc3c3c4cccc5c4n(c32)-c2ccccc2O5)cc1 ; c1ccc(-c2ccc3cc(-c4nc(-c5ccc6c(c5)c5c7cccc8c7n(c5n6-c5ccccc5)-c5ccccc5S8)nc(-c5cccc6ccccc56)n4)ccc3c2)cc1 ; c1ccc(-c2ccc(-c3nc(-c4ccc5ccccc5c4)nc(-c4cccc5c4c4c6cccc7c6n(c4n5-c4ccccc4)-c4ccccc4S7)n3)c3ccccc23)cc1]

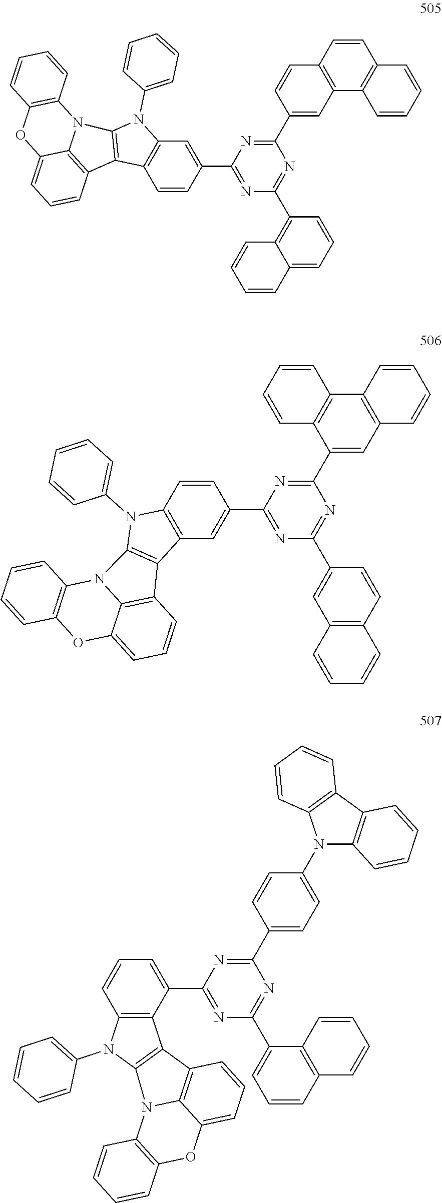 [SMILES: c1ccc(-n2c3cc(-c4nc(-c5ccc6ccc7ccccc7c6c5)nc(-c5cccc6ccccc56)n4)ccc3c3c4cccc5c4n(c32)-c2ccccc2O5)cc1 ; c1ccc(-n2c3ccc(-c4nc(-c5ccc6ccccc6c5)nc(-c5cc6ccccc6c6ccccc56)n4)cc3c3c4cccc5c4n(c32)-c2ccccc2O5)cc1 ; c1ccc(-n2c3cccc(-c4nc(-c5ccc(-n6c7ccccc7c7ccccc76)cc5)nc(-c5cccc6ccccc56)n4)c3c3c4cccc5c4n(c32)-c2ccccc2O5)cc1]

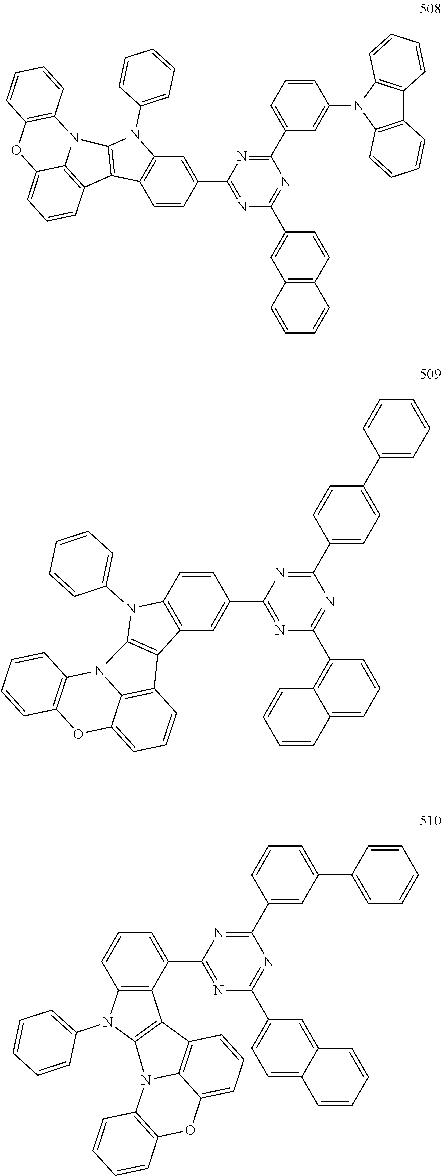 [SMILES: c1ccc(-n2c3cc(-c4nc(-c5cccc(-n6c7ccccc7c7ccccc76)c5)nc(-c5ccc6ccccc6c5)n4)ccc3c3c4cccc5c4n(c32)-c2ccccc2O5)cc1 ; c1ccc(-c2ccc(-c3nc(-c4ccc5c(c4)c4c6cccc7c6n(c4n5-c4ccccc4)-c4ccccc4O7)nc(-c4cccc5ccccc45)n3)cc2)cc1 ; c1ccc(-c2cccc(-c3nc(-c4ccc5ccccc5c4)nc(-c4cccc5c4c4c6cccc7c6n(c4n5-c4ccccc4)-c4ccccc4O7)n3)c2)cc1]

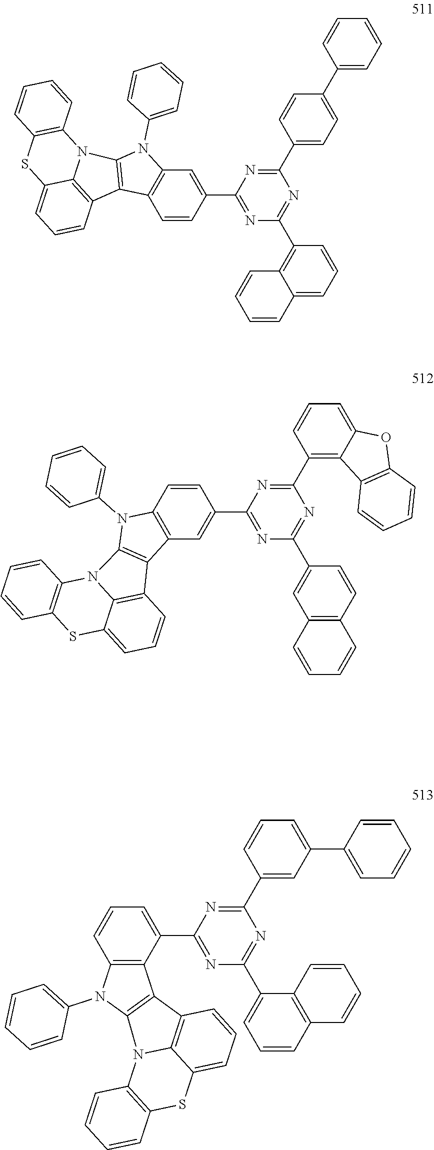 [SMILES: c1ccc(-c2ccc(-c3nc(-c4ccc5c6c7cccc8c7n(c6n(-c6ccccc6)c5c4)-c4ccccc4S8)nc(-c4cccc5ccccc45)n3)cc2)cc1 ; c1ccc(-n2c3ccc(-c4nc(-c5ccc6ccccc6c5)nc(-c5cccc6oc7ccccc7c56)n4)cc3c3c4cccc5c4n(c32)-c2ccccc2S5)cc1 ; c1ccc(-c2cccc(-c3nc(-c4cccc5ccccc45)nc(-c4cccc5c4c4c6cccc7c6n(c4n5-c4ccccc4)-c4ccccc4S7)n3)c2)cc1]

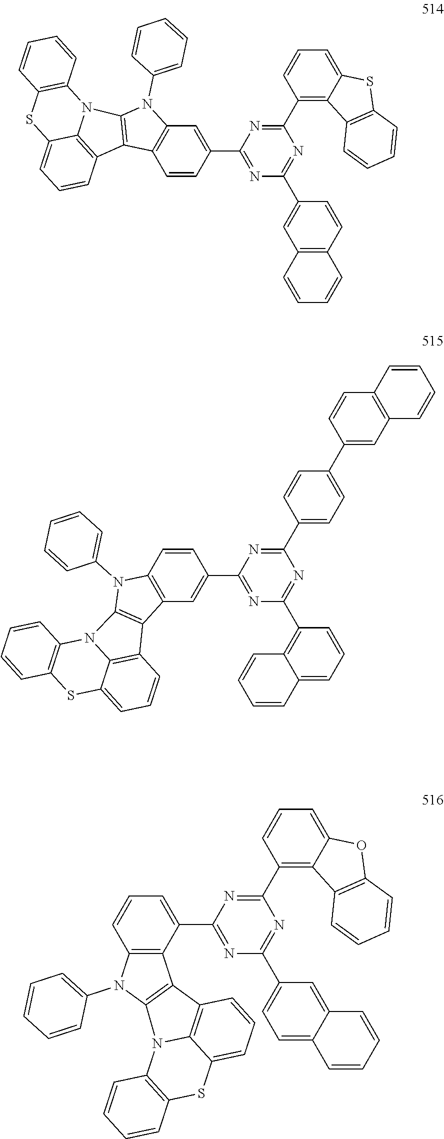 [SMILES: c1ccc(-n2c3cc(-c4nc(-c5ccc6ccccc6c5)nc(-c5cccc6sc7ccccc7c56)n4)ccc3c3c4cccc5c4n(c32)-c2ccccc2S5)cc1 ; c1ccc(-n2c3ccc(-c4nc(-c5ccc(-c6ccc7ccccc7c6)cc5)nc(-c5cccc6ccccc56)n4)cc3c3c4cccc5c4n(c32)-c2ccccc2S5)cc1 ; c1ccc(-n2c3cccc(-c4nc(-c5ccc6ccccc6c5)nc(-c5cccc6oc7ccccc7c56)n4)c3c3c4cccc5c4n(c32)-c2ccccc2S5)cc1]

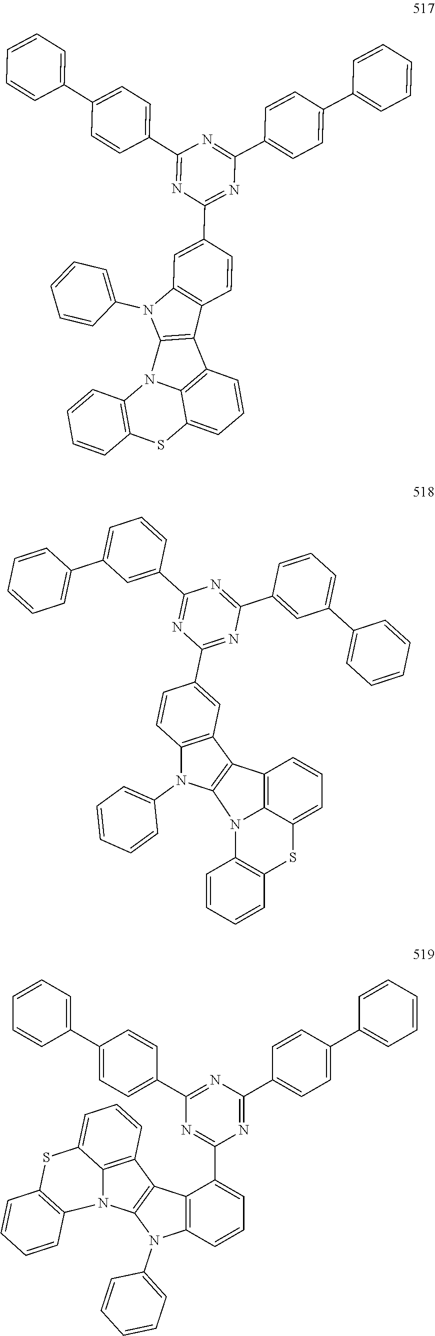 [SMILES: c1ccc(-c2ccc(-c3nc(-c4ccc(-c5ccccc5)cc4)nc(-c4ccc5c6c7cccc8c7n(c6n(-c6ccccc6)c5c4)-c4ccccc4S8)n3)cc2)cc1 ; c1ccc(-c2cccc(-c3nc(-c4cccc(-c5ccccc5)c4)nc(-c4ccc5c(c4)c4c6cccc7c6n(c4n5-c4ccccc4)-c4ccccc4S7)n3)c2)cc1 ; c1ccc(-c2ccc(-c3nc(-c4ccc(-c5ccccc5)cc4)nc(-c4cccc5c4c4c6cccc7c6n(c4n5-c4ccccc4)-c4ccccc4S7)n3)cc2)cc1]

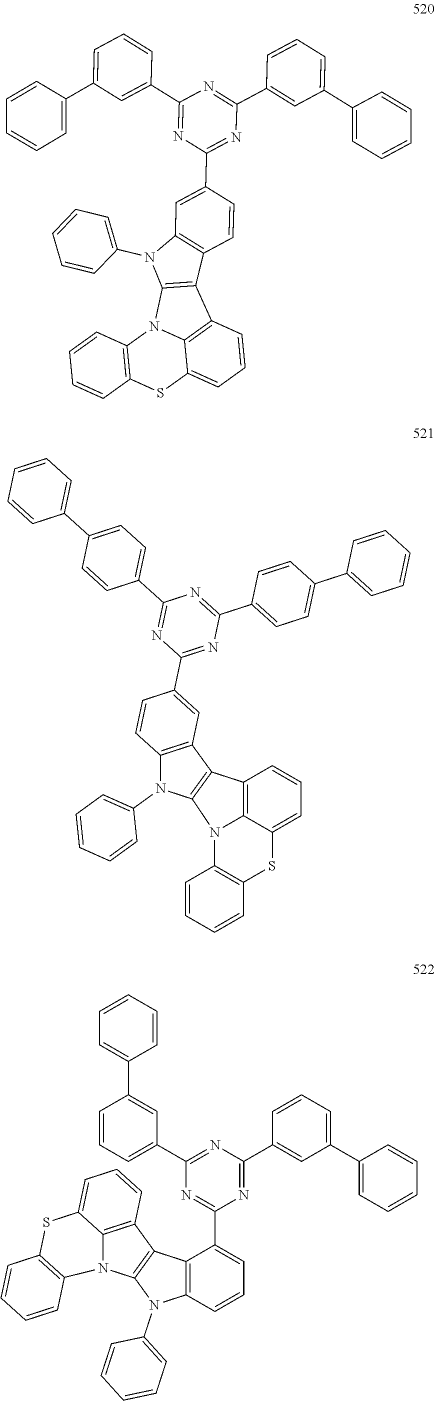 [SMILES: c1ccc(-c2cccc(-c3nc(-c4cccc(-c5ccccc5)c4)nc(-c4ccc5c6c7cccc8c7n(c6n(-c6ccccc6)c5c4)-c4ccccc4S8)n3)c2)cc1 ; c1ccc(-c2ccc(-c3nc(-c4ccc(-c5ccccc5)cc4)nc(-c4ccc5c(c4)c4c6cccc7c6n(c4n5-c4ccccc4)-c4ccccc4S7)n3)cc2)cc1 ; c1ccc(-c2cccc(-c3nc(-c4cccc(-c5ccccc5)c4)nc(-c4cccc5c4c4c6cccc7c6n(c4n5-c4ccccc4)-c4ccccc4S7)n3)c2)cc1]

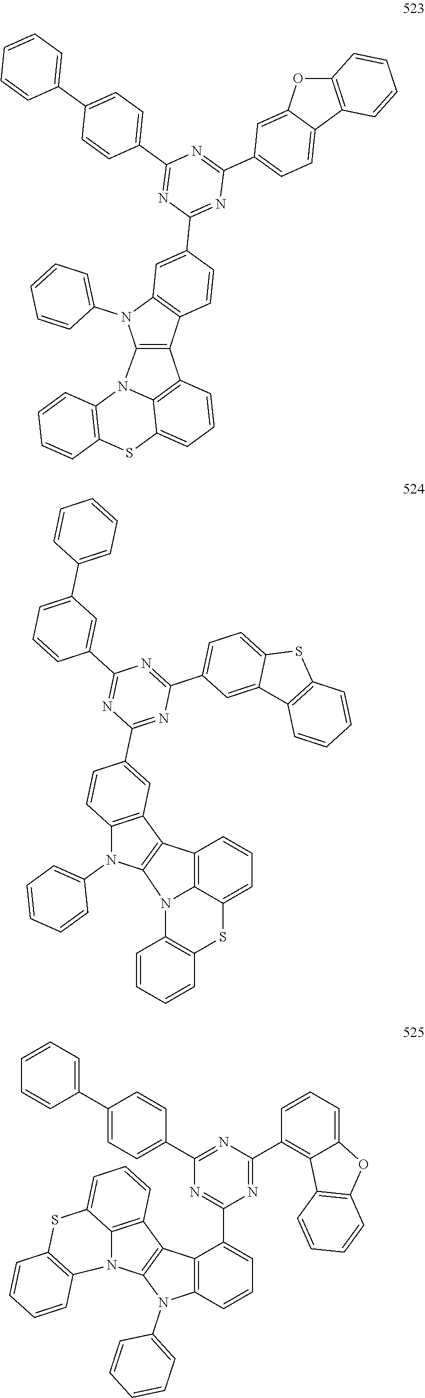

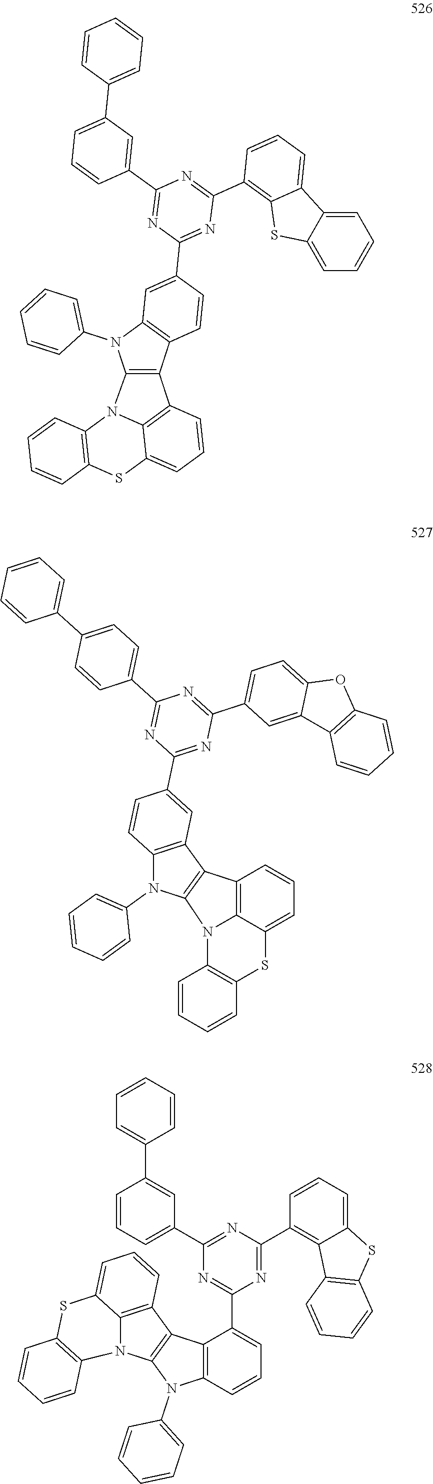 [SMILES: c1ccc(-c2cccc(-c3nc(-c4ccc5c6c7cccc8c7n(c6n(-c6ccccc6)c5c4)-c4ccccc4S8)nc(-c4cccc5c4sc4ccccc45)n3)c2)cc1 ; c1ccc(-c2ccc(-c3nc(-c4ccc5oc6ccccc6c5c4)nc(-c4ccc5c(c4)c4c6cccc7c6n(c4n5-c4ccccc4)-c4ccccc4S7)n3)cc2)cc1 ; c1ccc(-c2cccc(-c3nc(-c4cccc5sc6ccccc6c45)nc(-c4cccc5c4c4c6cccc7c6n(c4n5-c4ccccc4)-c4ccccc4S7)n3)c2)cc1]

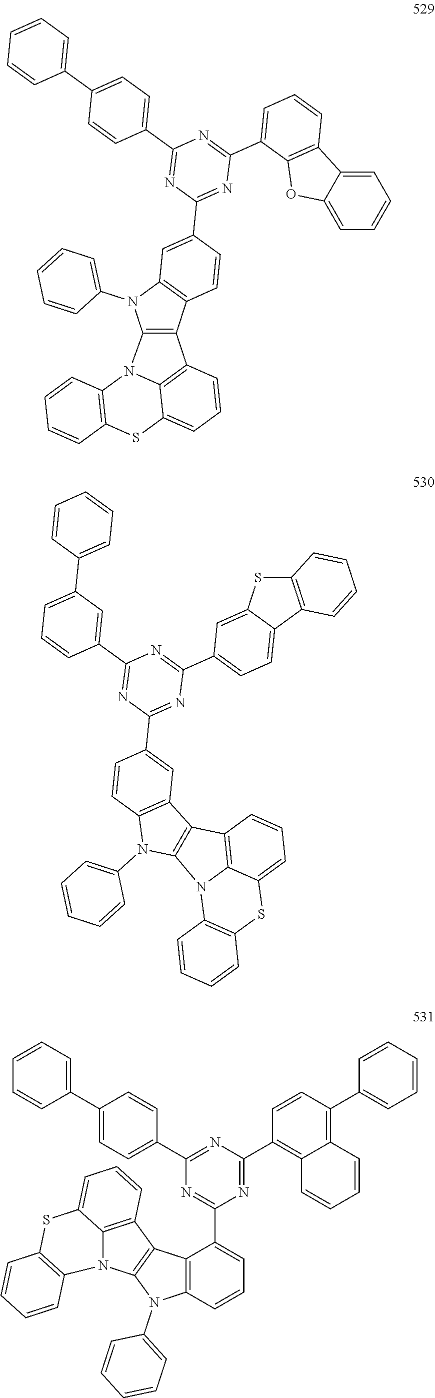 [SMILES: c1ccc(-c2ccc(-c3nc(-c4ccc5c6c7cccc8c7n(c6n(-c6ccccc6)c5c4)-c4ccccc4S8)nc(-c4cccc5c4oc4ccccc45)n3)cc2)cc1 ; c1ccc(-c2cccc(-c3nc(-c4ccc5c(c4)sc4ccccc45)nc(-c4ccc5c(c4)c4c6cccc7c6n(c4n5-c4ccccc4)-c4ccccc4S7)n3)c2)cc1 ; c1ccc(-c2ccc(-c3nc(-c4ccc(-c5ccccc5)c5ccccc45)nc(-c4cccc5c4c4c6cccc7c6n(c4n5-c4ccccc4)-c4ccccc4S7)n3)cc2)cc1]

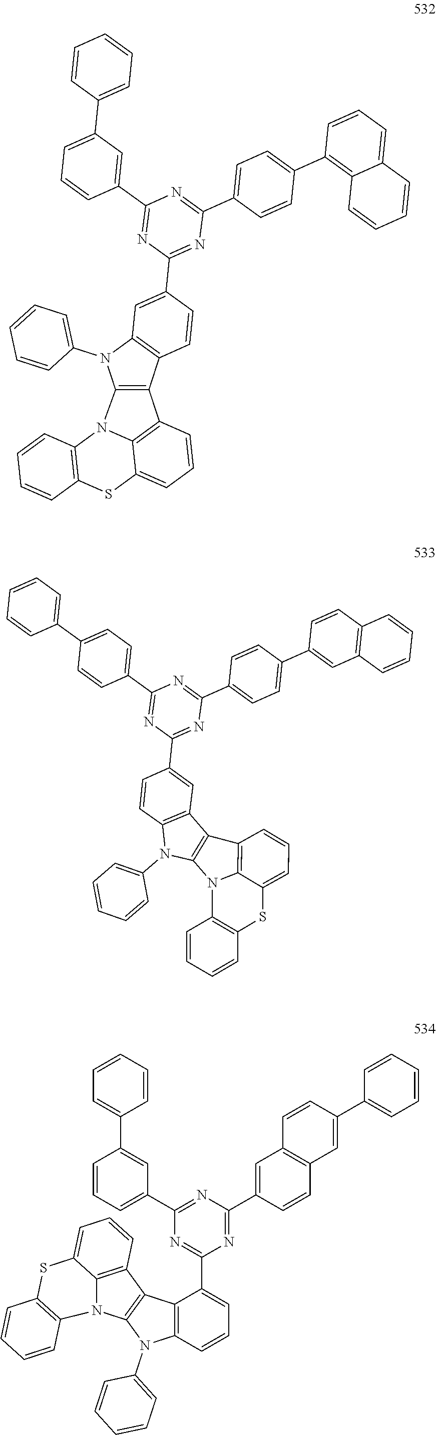 [SMILES: c1ccc(-c2cccc(-c3nc(-c4ccc(-c5cccc6ccccc56)cc4)nc(-c4ccc5c6c7cccc8c7n(c6n(-c6ccccc6)c5c4)-c4ccccc4S8)n3)c2)cc1 ; c1ccc(-c2ccc(-c3nc(-c4ccc(-c5ccc6ccccc6c5)cc4)nc(-c4ccc5c(c4)c4c6cccc7c6n(c4n5-c4ccccc4)-c4ccccc4S7)n3)cc2)cc1 ; c1ccc(-c2cccc(-c3nc(-c4ccc5cc(-c6ccccc6)ccc5c4)nc(-c4cccc5c4c4c6cccc7c6n(c4n5-c4ccccc4)-c4ccccc4S7)n3)c2)cc1]

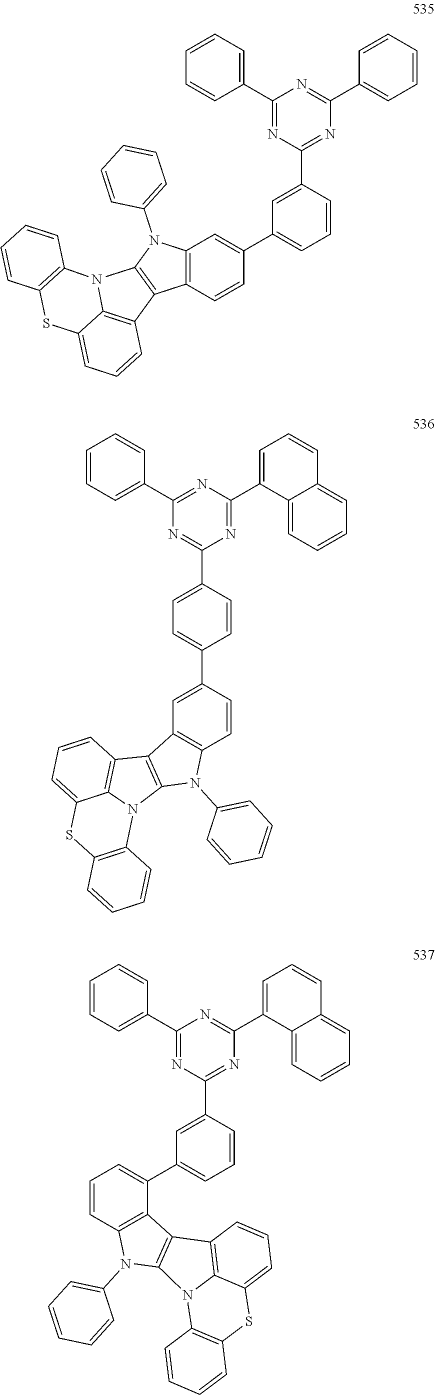 [SMILES: c1ccc(-c2nc(-c3ccccc3)nc(-c3cccc(-c4ccc5c6c7cccc8c7n(c6n(-c6ccccc6)c5c4)-c4ccccc4S8)c3)n2)cc1 ; c1ccc(-c2nc(-c3ccc(-c4ccc5c(c4)c4c6cccc7c6n(c4n5-c4ccccc4)-c4ccccc4S7)cc3)nc(-c3cccc4ccccc34)n2)cc1 ; c1ccc(-c2nc(-c3cccc(-c4cccc5c4c4c6cccc7c6n(c4n5-c4ccccc4)-c4ccccc4S7)c3)nc(-c3cccc4ccccc34)n2)cc1]

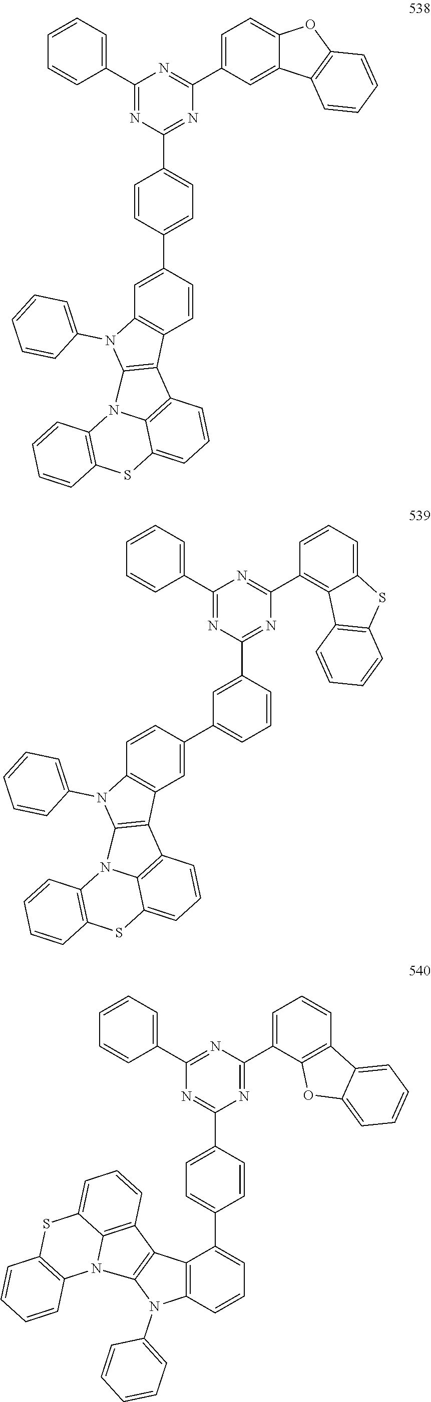 [SMILES: c1ccc(-c2nc(-c3ccc(-c4ccc5c6c7cccc8c7n(c6n(-c6ccccc6)c5c4)-c4ccccc4S8)cc3)nc(-c3ccc4oc5ccccc5c4c3)n2)cc1 ; c1ccc(-c2nc(-c3cccc(-c4ccc5c(c4)c4c6cccc7c6n(c4n5-c4ccccc4)-c4ccccc4S7)c3)nc(-c3cccc4sc5ccccc5c34)n2)cc1 ; c1ccc(-c2nc(-c3ccc(-c4cccc5c4c4c6cccc7c6n(c4n5-c4ccccc4)-c4ccccc4S7)cc3)nc(-c3cccc4c3oc3ccccc34)n2)cc1]

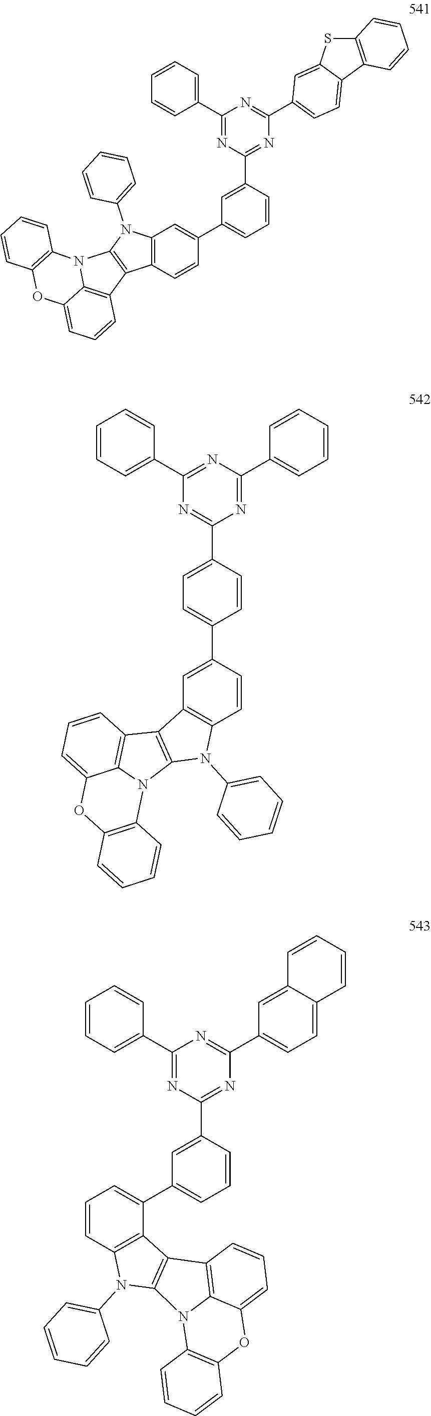 [SMILES: c1ccc(-c2nc(-c3cccc(-c4ccc5c6c7cccc8c7n(c6n(-c6ccccc6)c5c4)-c4ccccc4O8)c3)nc(-c3ccc4c(c3)sc3ccccc34)n2)cc1 ; c1ccc(-c2nc(-c3ccccc3)nc(-c3ccc(-c4ccc5c(c4)c4c6cccc7c6n(c4n5-c4ccccc4)-c4ccccc4O7)cc3)n2)cc1 ; c1ccc(-c2nc(-c3cccc(-c4cccc5c4c4c6cccc7c6n(c4n5-c4ccccc4)-c4ccccc4O7)c3)nc(-c3ccc4ccccc4c3)n2)cc1]

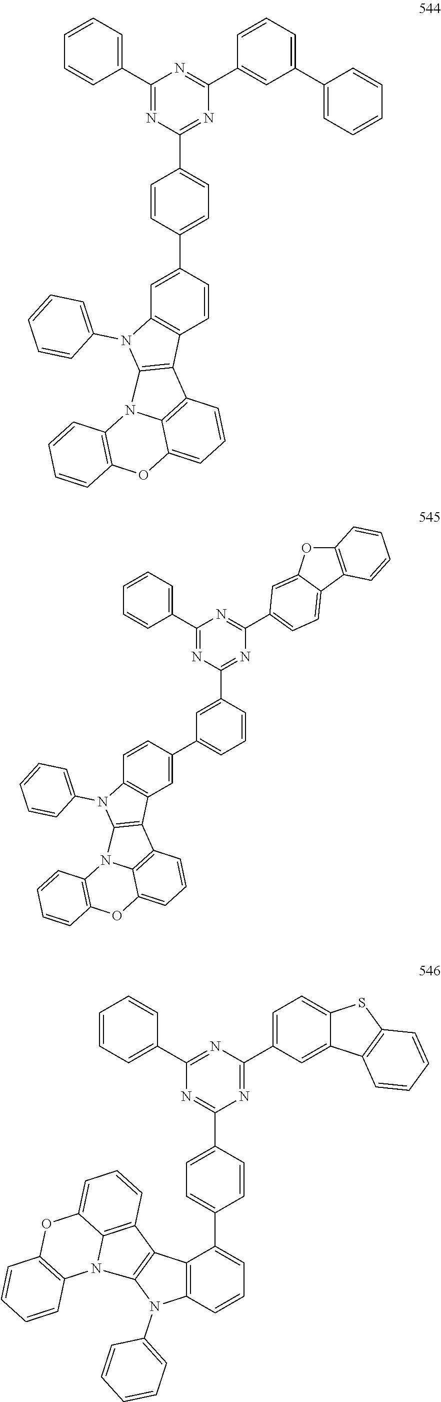 [SMILES: c1ccc(-c2cccc(-c3nc(-c4ccccc4)nc(-c4ccc(-c5ccc6c7c8cccc9c8n(c7n(-c7ccccc7)c6c5)-c5ccccc5O9)cc4)n3)c2)cc1 ; c1ccc(-c2nc(-c3cccc(-c4ccc5c(c4)c4c6cccc7c6n(c4n5-c4ccccc4)-c4ccccc4O7)c3)nc(-c3ccc4c(c3)oc3ccccc34)n2)cc1 ; c1ccc(-c2nc(-c3ccc(-c4cccc5c4c4c6cccc7c6n(c4n5-c4ccccc4)-c4ccccc4O7)cc3)nc(-c3ccc4sc5ccccc5c4c3)n2)cc1]

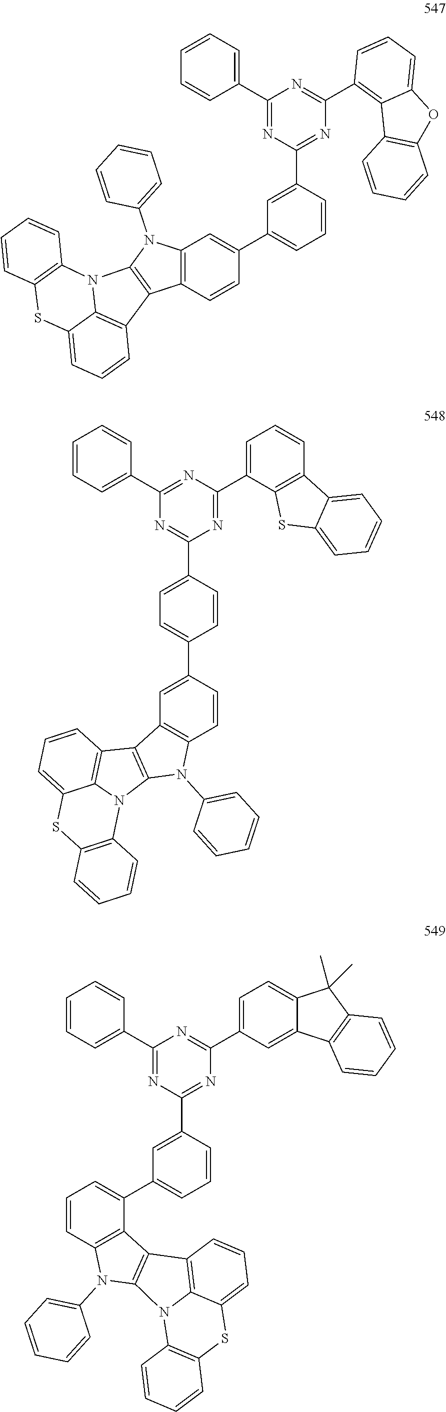 [SMILES: c1ccc(-c2nc(-c3cccc(-c4ccc5c6c7cccc8c7n(c6n(-c6ccccc6)c5c4)-c4ccccc4S8)c3)nc(-c3cccc4oc5ccccc5c34)n2)cc1 ; c1ccc(-c2nc(-c3ccc(-c4ccc5c(c4)c4c6cccc7c6n(c4n5-c4ccccc4)-c4ccccc4S7)cc3)nc(-c3cccc4c3sc3ccccc34)n2)cc1 ; CC1(C)c2ccccc2-c2cc(-c3nc(-c4ccccc4)nc(-c4cccc(-c5cccc6c5c5c7cccc8c7n(c5n6-c5ccccc5)-c5ccccc5S8)c4)n3)ccc21]

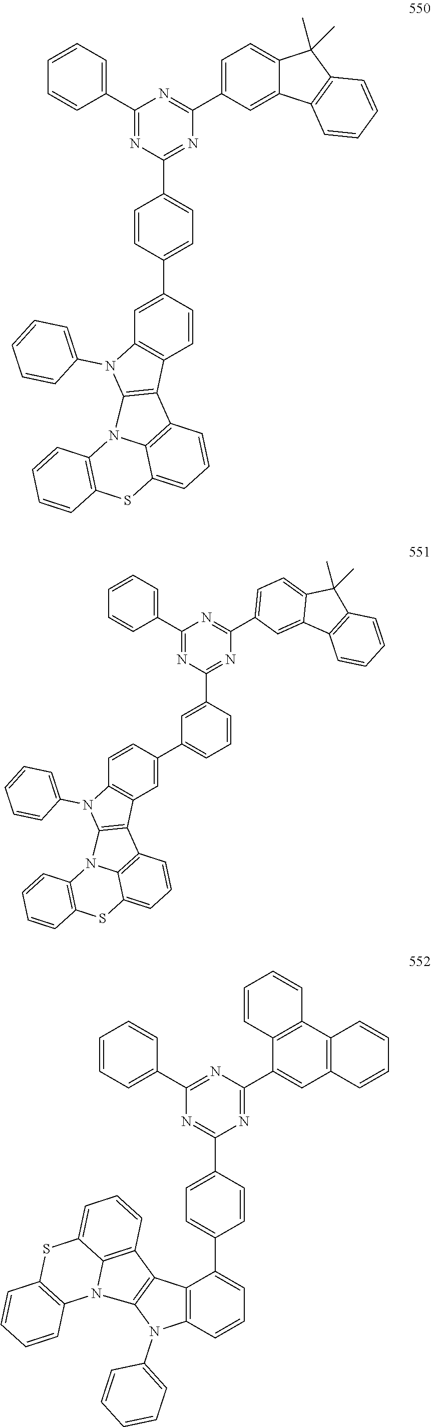 [SMILES: CC1(C)c2ccccc2-c2cc(-c3nc(-c4ccccc4)nc(-c4ccc(-c5ccc6c7c8cccc9c8n(c7n(-c7ccccc7)c6c5)-c5ccccc5S9)cc4)n3)ccc21 ; CC1(C)c2ccccc2-c2cc(-c3nc(-c4ccccc4)nc(-c4cccc(-c5ccc6c(c5)c5c7cccc8c7n(c5n6-c5ccccc5)-c5ccccc5S8)c4)n3)ccc21 ; c1ccc(-c2nc(-c3ccc(-c4cccc5c4c4c6cccc7c6n(c4n5-c4ccccc4)-c4ccccc4S7)cc3)nc(-c3cc4ccccc4c4ccccc34)n2)cc1]

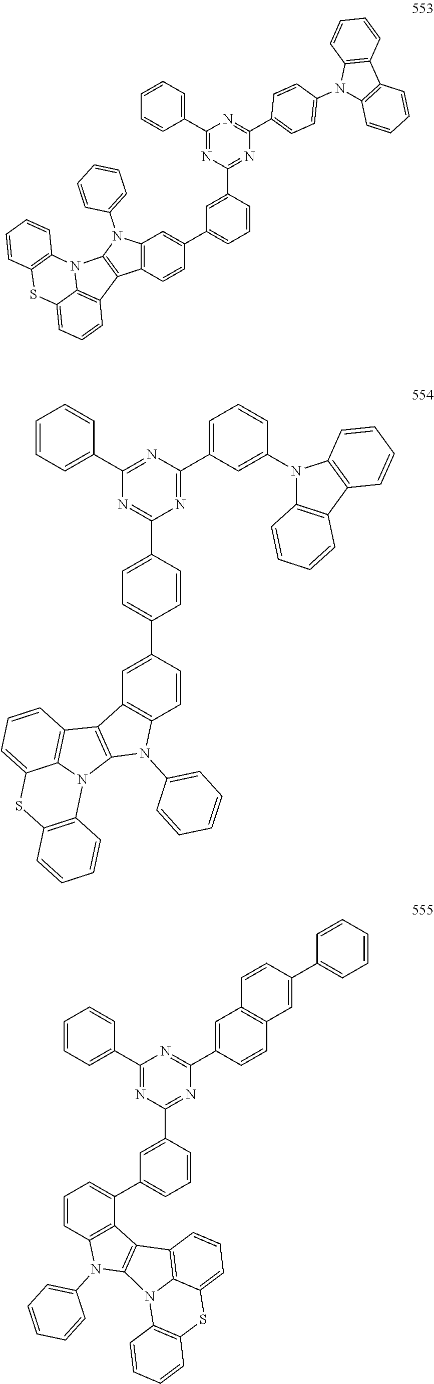 [SMILES: c1ccc(-c2nc(-c3ccc(-n4c5ccccc5c5ccccc54)cc3)nc(-c3cccc(-c4ccc5c6c7cccc8c7n(c6n(-c6ccccc6)c5c4)-c4ccccc4S8)c3)n2)cc1 ; c1ccc(-c2nc(-c3ccc(-c4ccc5c(c4)c4c6cccc7c6n(c4n5-c4ccccc4)-c4ccccc4S7)cc3)nc(-c3cccc(-n4c5ccccc5c5ccccc54)c3)n2)cc1 ; c1ccc(-c2ccc3cc(-c4nc(-c5ccccc5)nc(-c5cccc(-c6cccc7c6c6c8cccc9c8n(c6n7-c6ccccc6)-c6ccccc6S9)c5)n4)ccc3c2)cc1]

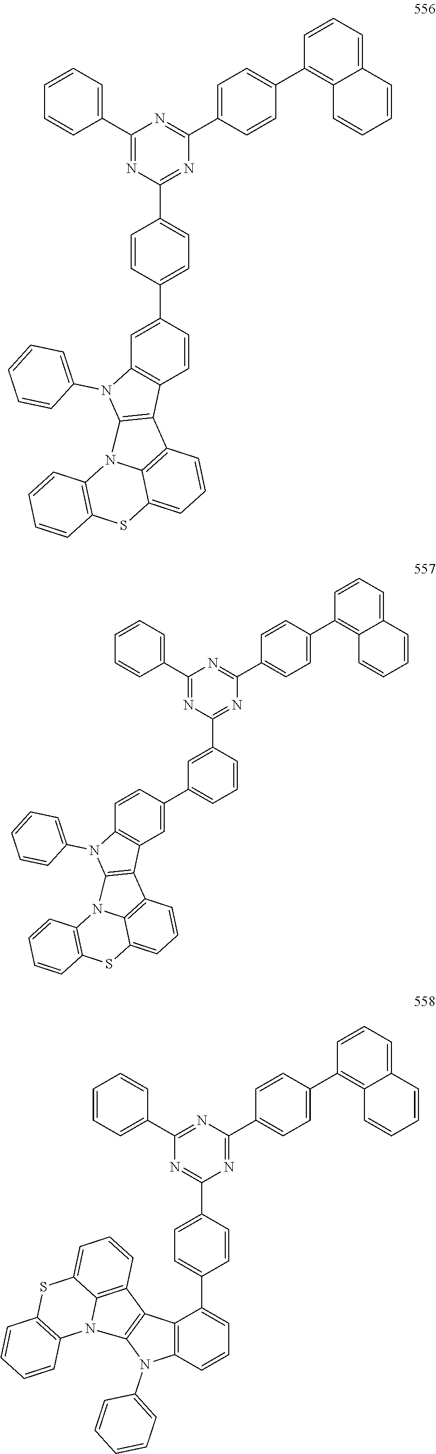 [SMILES: c1ccc(-c2nc(-c3ccc(-c4ccc5c6c7cccc8c7n(c6n(-c6ccccc6)c5c4)-c4ccccc4S8)cc3)nc(-c3ccc(-c4cccc5ccccc45)cc3)n2)cc1 ; c1ccc(-c2nc(-c3ccc(-c4cccc5ccccc45)cc3)nc(-c3cccc(-c4ccc5c(c4)c4c6cccc7c6n(c4n5-c4ccccc4)-c4ccccc4S7)c3)n2)cc1 ; c1ccc(-c2nc(-c3ccc(-c4cccc5ccccc45)cc3)nc(-c3ccc(-c4cccc5c4c4c6cccc7c6n(c4n5-c4ccccc4)-c4ccccc4S7)cc3)n2)cc1]

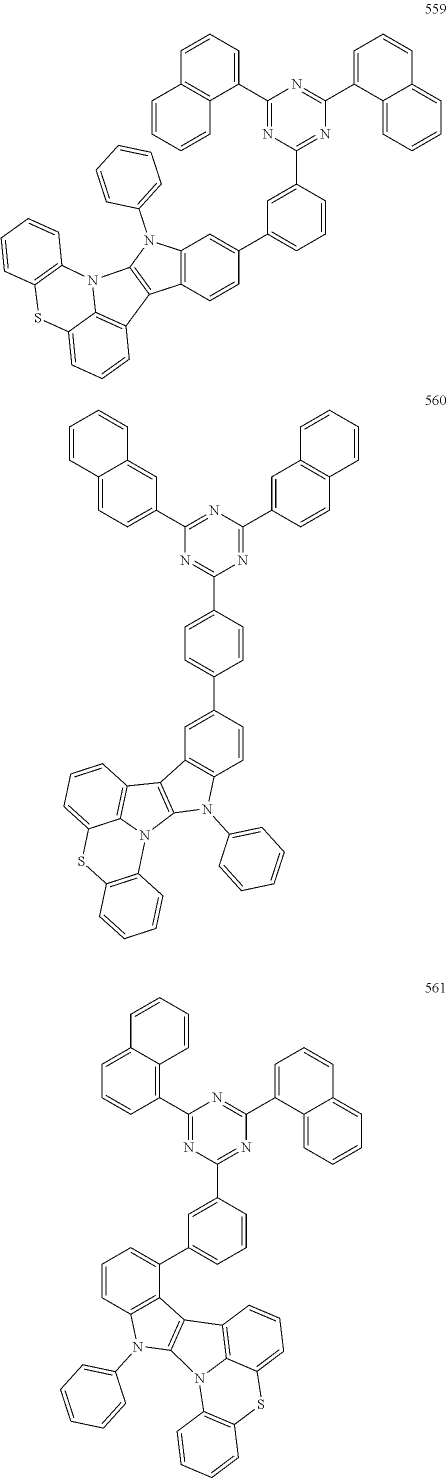

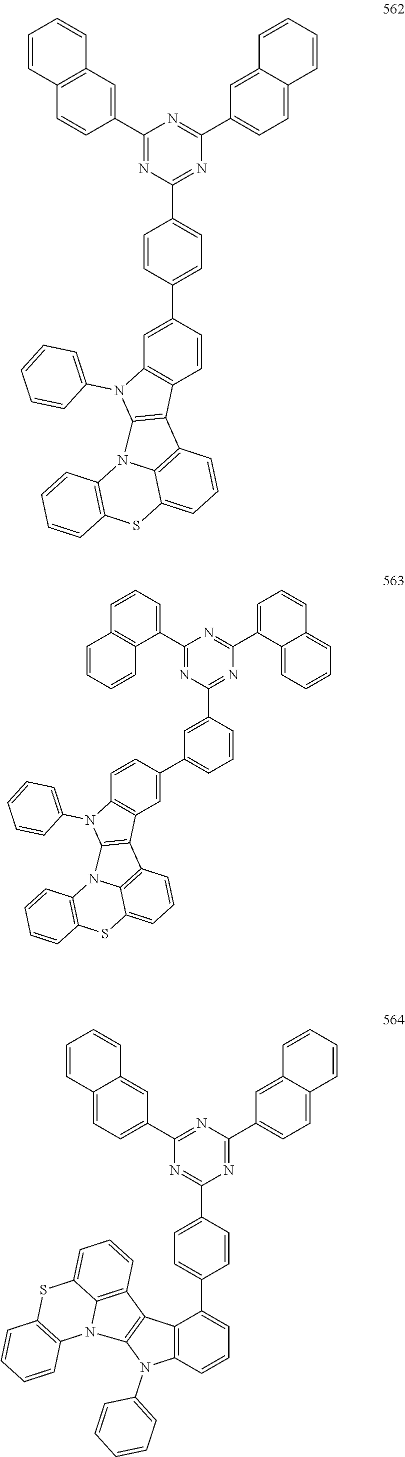 [SMILES: c1ccc(-n2c3cc(-c4ccc(-c5nc(-c6ccc7ccccc7c6)nc(-c6ccc7ccccc7c6)n5)cc4)ccc3c3c4cccc5c4n(c32)-c2ccccc2S5)cc1 ; c1ccc(-n2c3ccc(-c4cccc(-c5nc(-c6cccc7ccccc67)nc(-c6cccc7ccccc67)n5)c4)cc3c3c4cccc5c4n(c32)-c2ccccc2S5)cc1 ; c1ccc(-n2c3cccc(-c4ccc(-c5nc(-c6ccc7ccccc7c6)nc(-c6ccc7ccccc7c6)n5)cc4)c3c3c4cccc5c4n(c32)-c2ccccc2S5)cc1]

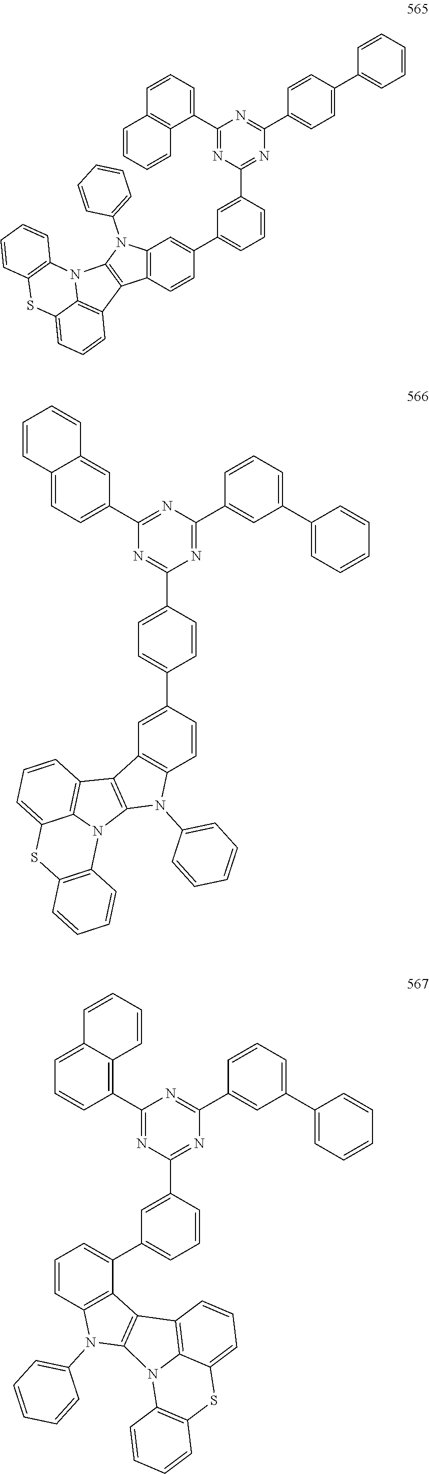 [SMILES: c1ccc(-c2ccc(-c3nc(-c4cccc(-c5ccc6c7c8cccc9c8n(c7n(-c7ccccc7)c6c5)-c5ccccc5S9)c4)nc(-c4cccc5ccccc45)n3)cc2)cc1 ; c1ccc(-c2cccc(-c3nc(-c4ccc(-c5ccc6c(c5)c5c7cccc8c7n(c5n6-c5ccccc5)-c5ccccc5S8)cc4)nc(-c4ccc5ccccc5c4)n3)c2)cc1 ; c1ccc(-c2cccc(-c3nc(-c4cccc(-c5cccc6c5c5c7cccc8c7n(c5n6-c5ccccc5)-c5ccccc5S8)c4)nc(-c4cccc5ccccc45)n3)c2)cc1]

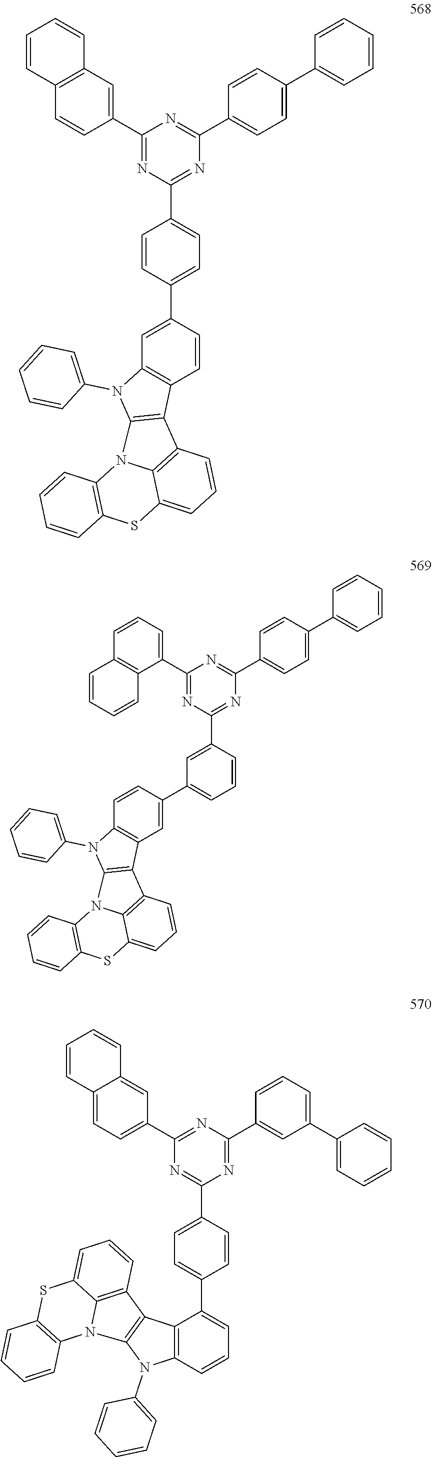 [SMILES: c1ccc(-c2ccc(-c3nc(-c4ccc(-c5ccc6c7c8cccc9c8n(c7n(-c7ccccc7)c6c5)-c5ccccc5S9)cc4)nc(-c4ccc5ccccc5c4)n3)cc2)cc1 ; c1ccc(-c2ccc(-c3nc(-c4cccc(-c5ccc6c(c5)c5c7cccc8c7n(c5n6-c5ccccc5)-c5ccccc5S8)c4)nc(-c4cccc5ccccc45)n3)cc2)cc1 ; c1ccc(-c2cccc(-c3nc(-c4ccc(-c5cccc6c5c5c7cccc8c7n(c5n6-c5ccccc5)-c5ccccc5S8)cc4)nc(-c4ccc5ccccc5c4)n3)c2)cc1]

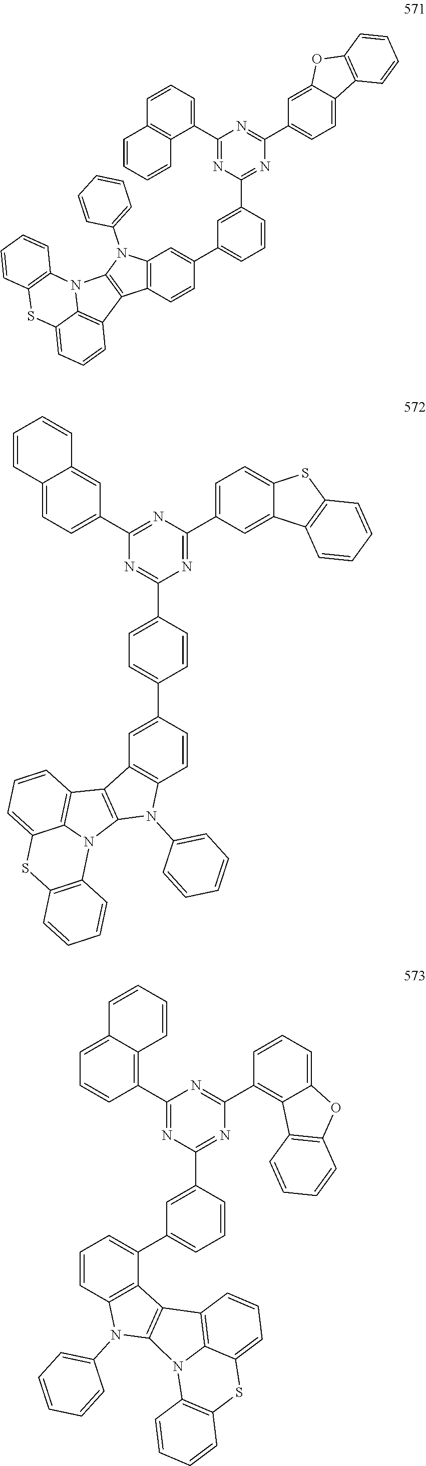 [SMILES: c1ccc(-n2c3cc(-c4cccc(-c5nc(-c6ccc7c(c6)oc6ccccc67)nc(-c6cccc7ccccc67)n5)c4)ccc3c3c4cccc5c4n(c32)-c2ccccc2S5)cc1 ; c1ccc(-n2c3ccc(-c4ccc(-c5nc(-c6ccc7ccccc7c6)nc(-c6ccc7sc8ccccc8c7c6)n5)cc4)cc3c3c4cccc5c4n(c32)-c2ccccc2S5)cc1 ; c1ccc(-n2c3cccc(-c4cccc(-c5nc(-c6cccc7ccccc67)nc(-c6cccc7oc8ccccc8c67)n5)c4)c3c3c4cccc5c4n(c32)-c2ccccc2S5)cc1]

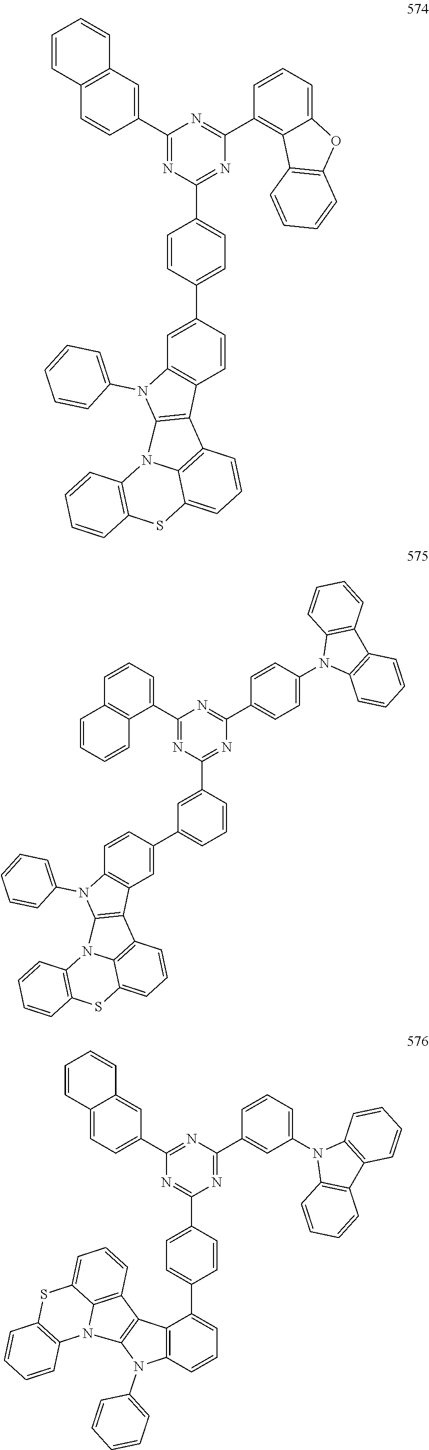 [SMILES: c1ccc(-n2c3cc(-c4ccc(-c5nc(-c6ccc7ccccc7c6)nc(-c6cccc7oc8ccccc8c67)n5)cc4)ccc3c3c4cccc5c4n(c32)-c2ccccc2S5)cc1 ; c1ccc(-n2c3ccc(-c4cccc(-c5nc(-c6ccc(-n7c8ccccc8c8ccccc87)cc6)nc(-c6cccc7ccccc67)n5)c4)cc3c3c4cccc5c4n(c32)-c2ccccc2S5)cc1 ; c1ccc(-n2c3cccc(-c4ccc(-c5nc(-c6cccc(-n7c8ccccc8c8ccccc87)c6)nc(-c6ccc7ccccc7c6)n5)cc4)c3c3c4cccc5c4n(c32)-c2ccccc2S5)cc1]

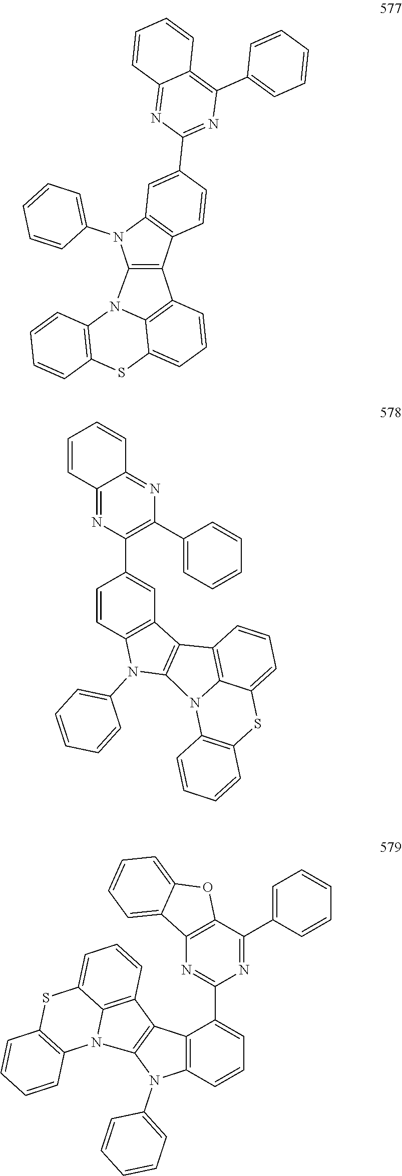 [SMILES: c1ccc(-c2nc(-c3ccc4c5c6cccc7c6n(c5n(-c5ccccc5)c4c3)-c3ccccc3S7)nc3ccccc23)cc1 ; c1ccc(-c2nc3ccccc3nc2-c2ccc3c(c2)c2c4cccc5c4n(c2n3-c2ccccc2)-c2ccccc2S5)cc1 ; c1ccc(-c2nc(-c3cccc4c3c3c5cccc6c5n(c3n4-c3ccccc3)-c3ccccc3S6)nc3c2oc2ccccc23)cc1]

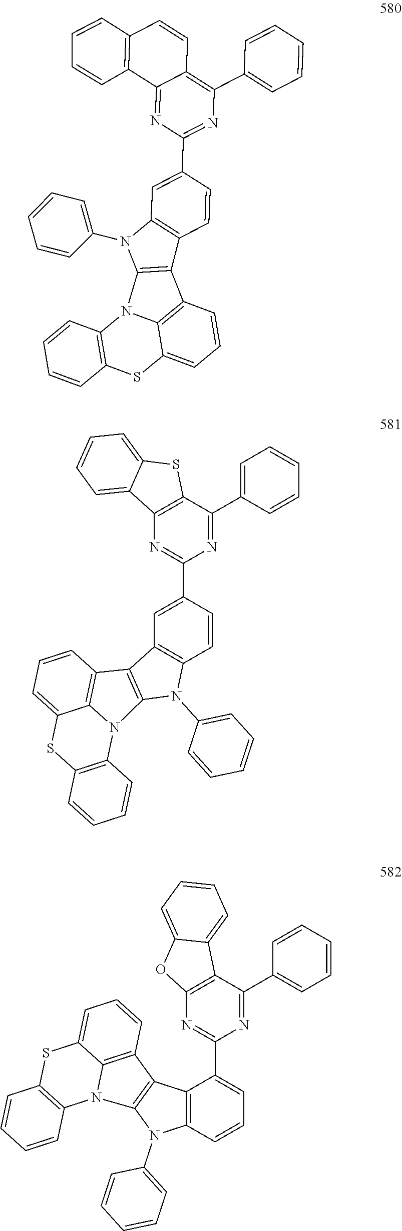 [SMILES: c1ccc(-c2nc(-c3ccc4c5c6cccc7c6n(c5n(-c5ccccc5)c4c3)-c3ccccc3S7)nc3c2ccc2ccccc23)cc1 ; c1ccc(-c2nc(-c3ccc4c(c3)c3c5cccc6c5n(c3n4-c3ccccc3)-c3ccccc3S6)nc3c2sc2ccccc23)cc1 ; c1ccc(-c2nc(-c3cccc4c3c3c5cccc6c5n(c3n4-c3ccccc3)-c3ccccc3S6)nc3oc4ccccc4c23)cc1]

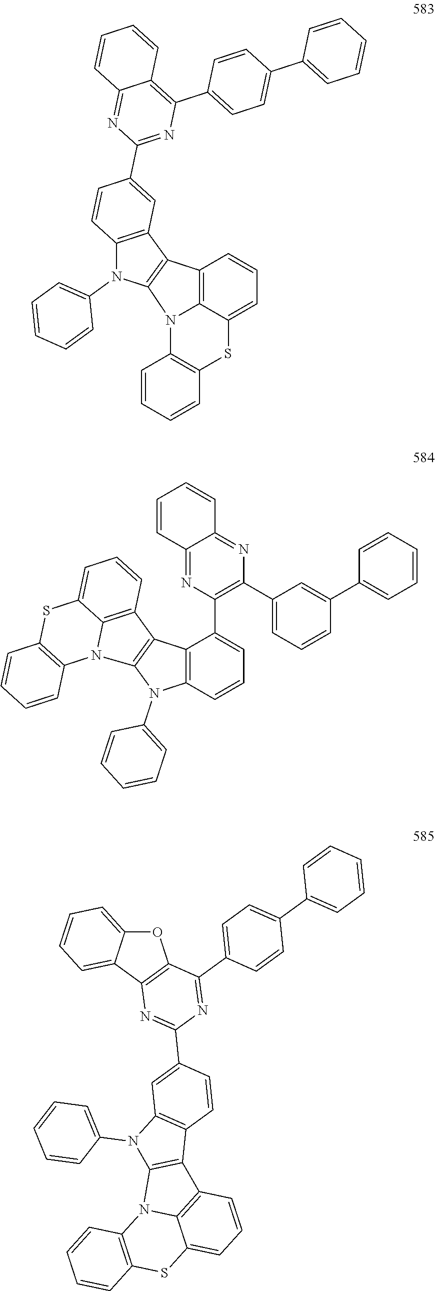 [SMILES: c1ccc(-c2ccc(-c3nc(-c4ccc5c(c4)c4c6cccc7c6n(c4n5-c4ccccc4)-c4ccccc4S7)nc4ccccc34)cc2)cc1 ; c1ccc(-c2cccc(-c3nc4ccccc4nc3-c3cccc4c3c3c5cccc6c5n(c3n4-c3ccccc3)-c3ccccc3S6)c2)cc1 ; c1ccc(-c2ccc(-c3nc(-c4ccc5c6c7cccc8c7n(c6n(-c6ccccc6)c5c4)-c4ccccc4S8)nc4c3oc3ccccc34)cc2)cc1]

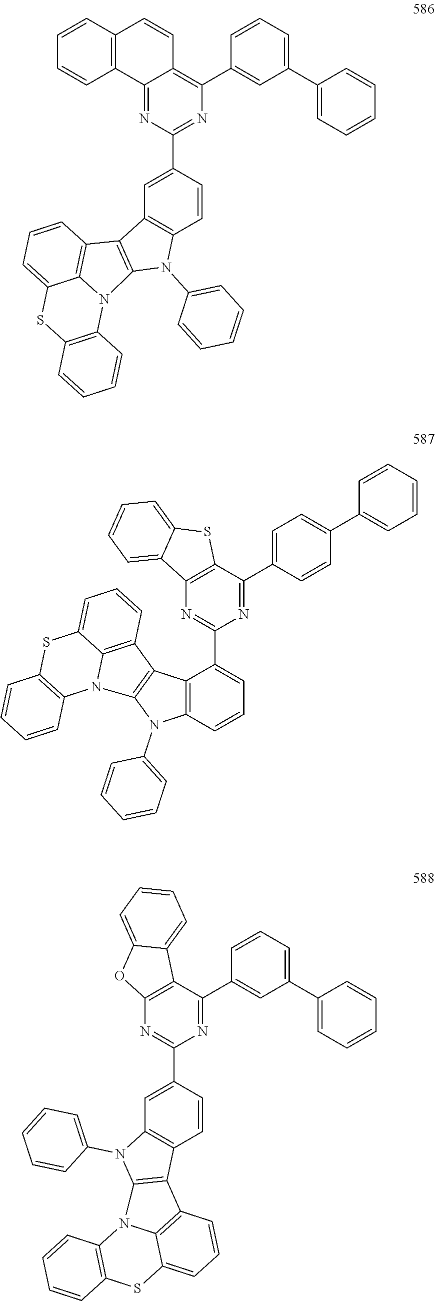 [SMILES: c1ccc(-c2cccc(-c3nc(-c4ccc5c(c4)c4c6cccc7c6n(c4n5-c4ccccc4)-c4ccccc4S7)nc4c3ccc3ccccc34)c2)cc1 ; c1ccc(-c2ccc(-c3nc(-c4cccc5c4c4c6cccc7c6n(c4n5-c4ccccc4)-c4ccccc4S7)nc4c3sc3ccccc34)cc2)cc1 ; c1ccc(-c2cccc(-c3nc(-c4ccc5c6c7cccc8c7n(c6n(-c6ccccc6)c5c4)-c4ccccc4S8)nc4oc5ccccc5c34)c2)cc1]

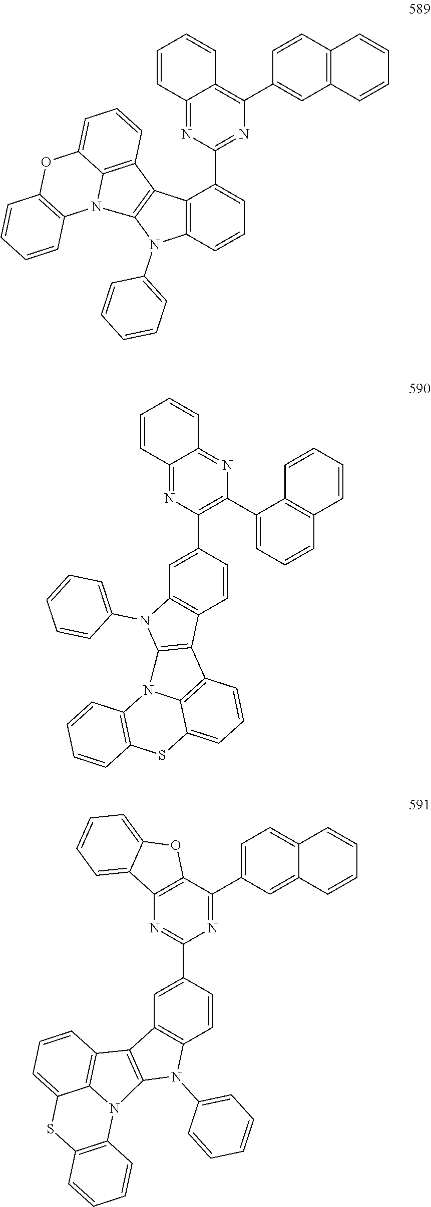 [SMILES: c1ccc(-n2c3cccc(-c4nc(-c5ccc6ccccc6c5)c5ccccc5n4)c3c3c4cccc5c4n(c32)-c2ccccc2O5)cc1 ; c1ccc(-n2c3cc(-c4nc5ccccc5nc4-c4cccc5ccccc45)ccc3c3c4cccc5c4n(c32)-c2ccccc2S5)cc1 ; c1ccc(-n2c3ccc(-c4nc(-c5ccc6ccccc6c5)c5oc6ccccc6c5n4)cc3c3c4cccc5c4n(c32)-c2ccccc2S5)cc1]

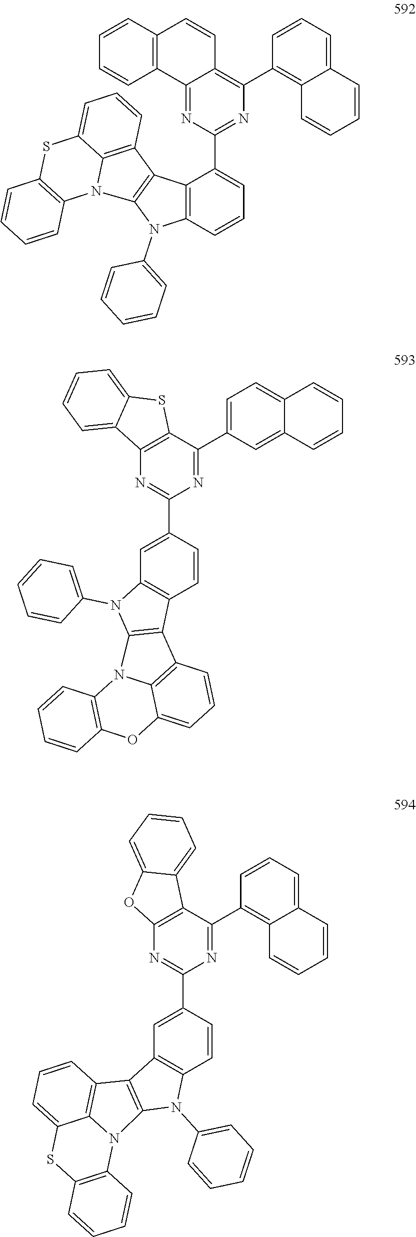 [SMILES: c1ccc(-n2c3cccc(-c4nc(-c5cccc6ccccc56)c5ccc6ccccc6c5n4)c3c3c4cccc5c4n(c32)-c2ccccc2S5)cc1 ; c1ccc(-n2c3cc(-c4nc(-c5ccc6ccccc6c5)c5sc6ccccc6c5n4)ccc3c3c4cccc5c4n(c32)-c2ccccc2O5)cc1 ; c1ccc(-n2c3ccc(-c4nc(-c5cccc6ccccc56)c5c(n4)oc4ccccc45)cc3c3c4cccc5c4n(c32)-c2ccccc2S5)cc1]

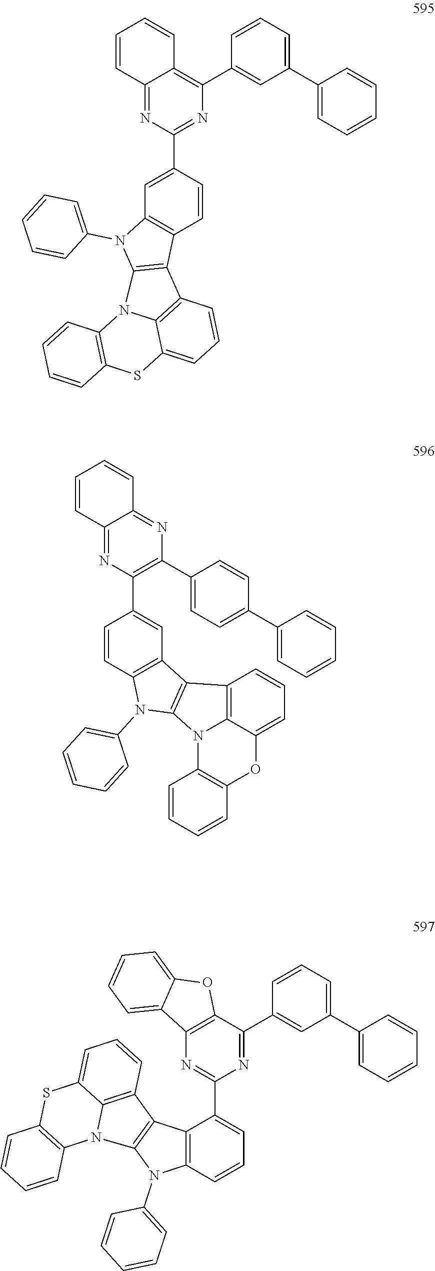 [SMILES: c1ccc(-c2cccc(-c3nc(-c4ccc5c6c7cccc8c7n(c6n(-c6ccccc6)c5c4)-c4ccccc4S8)nc4ccccc34)c2)cc1 ; c1ccc(-c2ccc(-c3nc4ccccc4nc3-c3ccc4c(c3)c3c5cccc6c5n(c3n4-c3ccccc3)-c3ccccc3O6)cc2)cc1 ; c1ccc(-c2cccc(-c3nc(-c4cccc5c4c4c6cccc7c6n(c4n5-c4ccccc4)-c4ccccc4S7)nc4c3oc3ccccc34)c2)cc1]

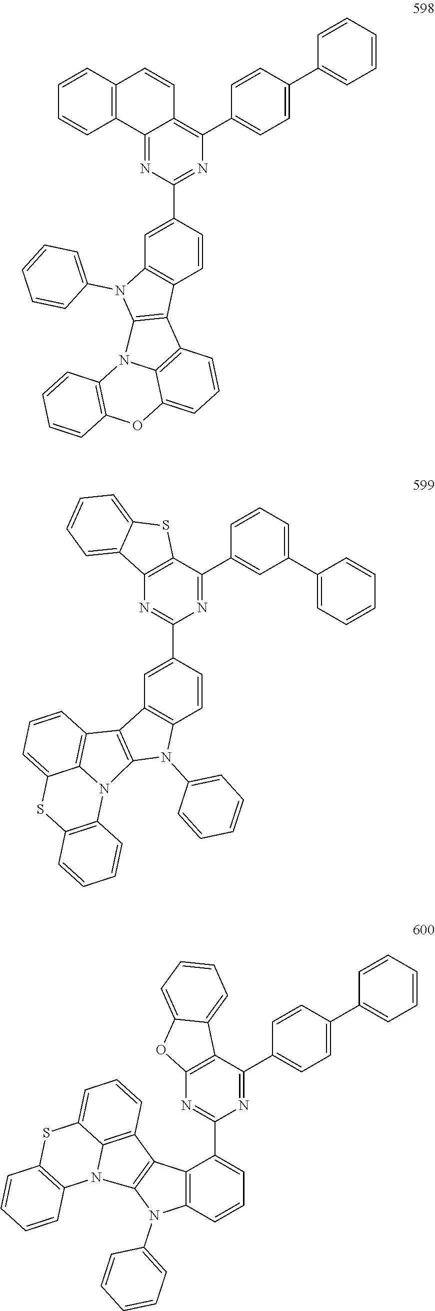 [SMILES: c1ccc(-c2ccc(-c3nc(-c4ccc5c6c7cccc8c7n(c6n(-c6ccccc6)c5c4)-c4ccccc4O8)nc4c3ccc3ccccc34)cc2)cc1 ; c1ccc(-c2cccc(-c3nc(-c4ccc5c(c4)c4c6cccc7c6n(c4n5-c4ccccc4)-c4ccccc4S7)nc4c3sc3ccccc34)c2)cc1 ; c1ccc(-c2ccc(-c3nc(-c4cccc5c4c4c6cccc7c6n(c4n5-c4ccccc4)-c4ccccc4S7)nc4oc5ccccc5c34)cc2)cc1]

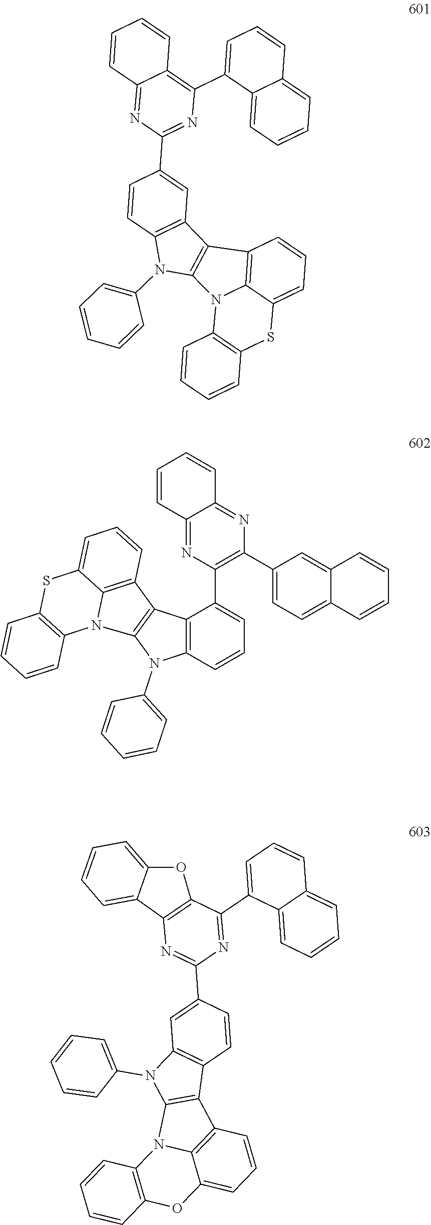 [SMILES: c1ccc(-n2c3ccc(-c4nc(-c5cccc6ccccc56)c5ccccc5n4)cc3c3c4cccc5c4n(c32)-c2ccccc2S5)cc1 ; c1ccc(-n2c3cccc(-c4nc5ccccc5nc4-c4ccc5ccccc5c4)c3c3c4cccc5c4n(c32)-c2ccccc2S5)cc1 ; c1ccc(-n2c3cc(-c4nc(-c5cccc6ccccc56)c5oc6ccccc6c5n4)ccc3c3c4cccc5c4n(c32)-c2ccccc2O5)cc1]

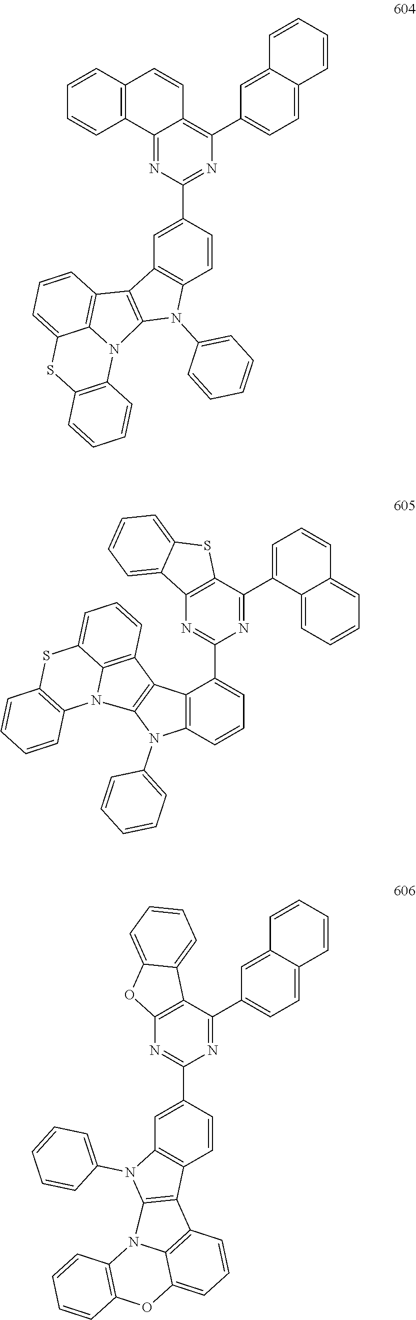 [SMILES: c1ccc(-n2c3ccc(-c4nc(-c5ccc6ccccc6c5)c5ccc6ccccc6c5n4)cc3c3c4cccc5c4n(c32)-c2ccccc2S5)cc1 ; c1ccc(-n2c3cccc(-c4nc(-c5cccc6ccccc56)c5sc6ccccc6c5n4)c3c3c4cccc5c4n(c32)-c2ccccc2S5)cc1 ; c1ccc(-n2c3cc(-c4nc(-c5ccc6ccccc6c5)c5c(n4)oc4ccccc45)ccc3c3c4cccc5c4n(c32)-c2ccccc2O5)cc1]

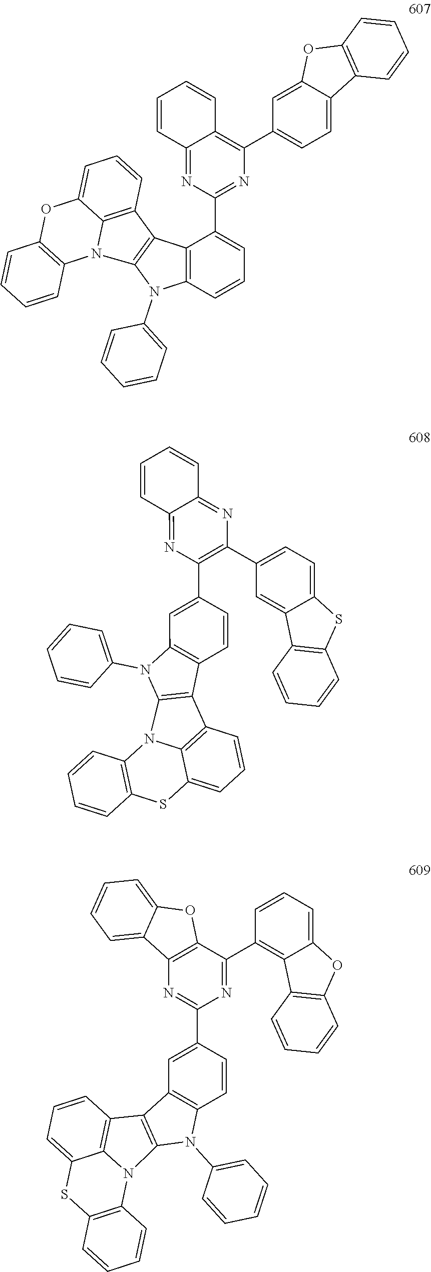 [SMILES: c1ccc(-n2c3cccc(-c4nc(-c5ccc6c(c5)oc5ccccc56)c5ccccc5n4)c3c3c4cccc5c4n(c32)-c2ccccc2O5)cc1 ; c1ccc(-n2c3cc(-c4nc5ccccc5nc4-c4ccc5sc6ccccc6c5c4)ccc3c3c4cccc5c4n(c32)-c2ccccc2S5)cc1 ; c1ccc(-n2c3ccc(-c4nc(-c5cccc6oc7ccccc7c56)c5oc6ccccc6c5n4)cc3c3c4cccc5c4n(c32)-c2ccccc2S5)cc1]

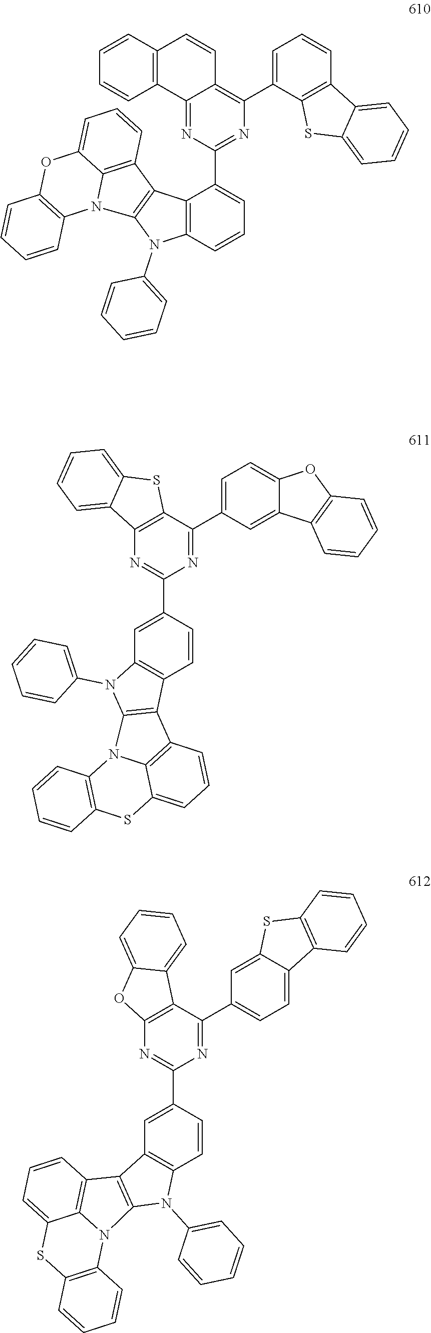 [SMILES: c1ccc(-n2c3cccc(-c4nc(-c5cccc6c5sc5ccccc56)c5ccc6ccccc6c5n4)c3c3c4cccc5c4n(c32)-c2ccccc2O5)cc1 ; c1ccc(-n2c3cc(-c4nc(-c5ccc6oc7ccccc7c6c5)c5sc6ccccc6c5n4)ccc3c3c4cccc5c4n(c32)-c2ccccc2S5)cc1 ; c1ccc(-n2c3ccc(-c4nc(-c5ccc6c(c5)sc5ccccc56)c5c(n4)oc4ccccc45)cc3c3c4cccc5c4n(c32)-c2ccccc2S5)cc1]

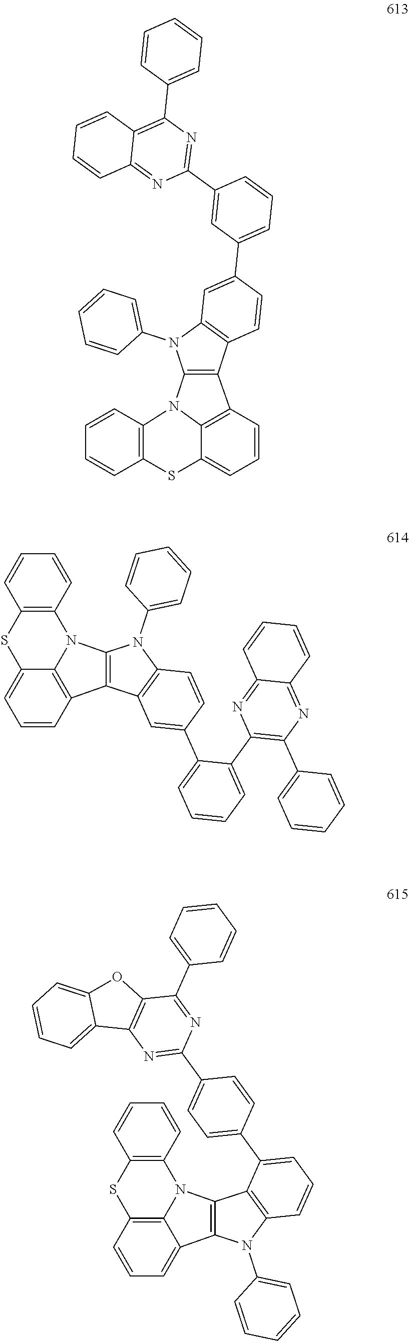 [SMILES: c1ccc(-c2nc(-c3cccc(-c4ccc5c6c7cccc8c7n(c6n(-c6ccccc6)c5c4)-c4ccccc4S8)c3)nc3ccccc23)cc1 ; c1ccc(-c2nc3ccccc3nc2-c2ccccc2-c2ccc3c(c2)c2c4cccc5c4n(c2n3-c2ccccc2)-c2ccccc2S5)cc1 ; c1ccc(-c2nc(-c3ccc(-c4cccc5c4c4c(c6cccc7c6n4-c4ccccc4S7)n5-c4ccccc4)cc3)nc3c2oc2ccccc23)cc1]

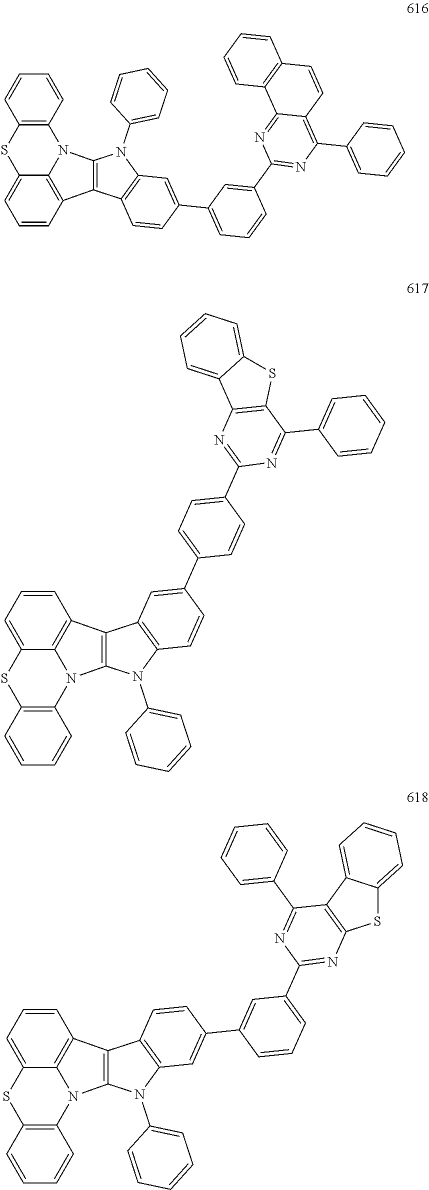 [SMILES: c1ccc(-c2nc(-c3cccc(-c4ccc5c6c7cccc8c7n(c6n(-c6ccccc6)c5c4)-c4ccccc4S8)c3)nc3c2ccc2ccccc23)cc1 ; c1ccc(-c2nc(-c3ccc(-c4ccc5c(c4)c4c6cccc7c6n(c4n5-c4ccccc4)-c4ccccc4S7)cc3)nc3c2sc2ccccc23)cc1 ; c1ccc(-c2nc(-c3cccc(-c4ccc5c6c7cccc8c7n(c6n(-c6ccccc6)c5c4)-c4ccccc4S8)c3)nc3sc4ccccc4c23)cc1]

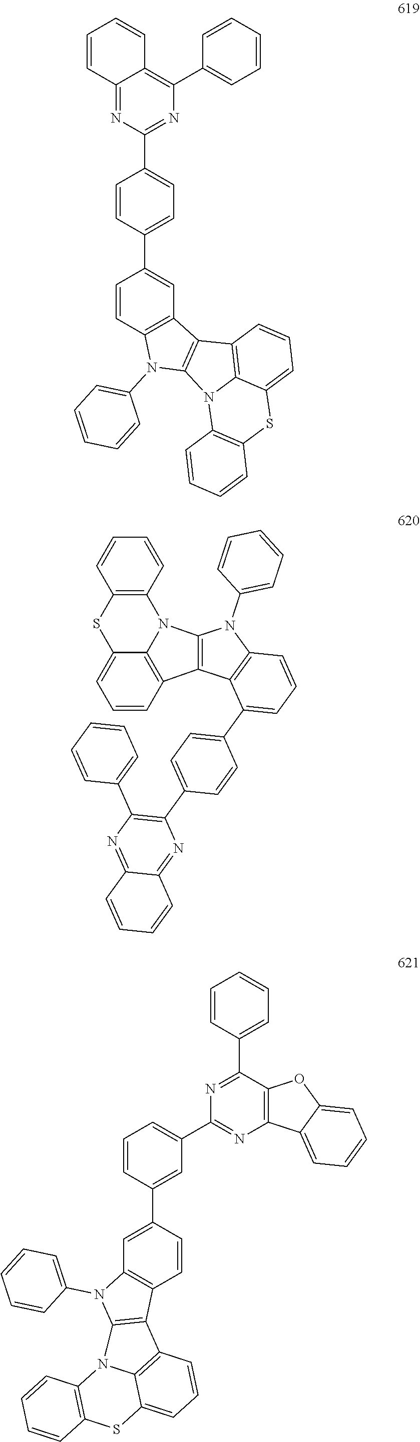

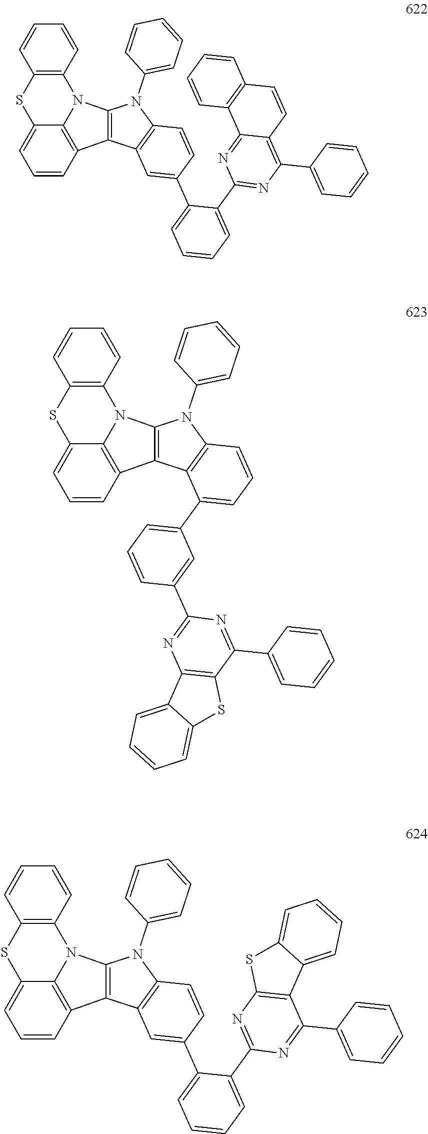 [SMILES: c1ccc(-c2nc(-c3ccccc3-c3ccc4c(c3)c3c5cccc6c5n(c3n4-c3ccccc3)-c3ccccc3S6)nc3c2ccc2ccccc23)cc1 ; c1ccc(-c2nc(-c3cccc(-c4cccc5c4c4c6cccc7c6n(c4n5-c4ccccc4)-c4ccccc4S7)c3)nc3c2sc2ccccc23)cc1 ; c1ccc(-c2nc(-c3ccccc3-c3ccc4c(c3)c3c5cccc6c5n(c3n4-c3ccccc3)-c3ccccc3S6)nc3sc4ccccc4c23)cc1]

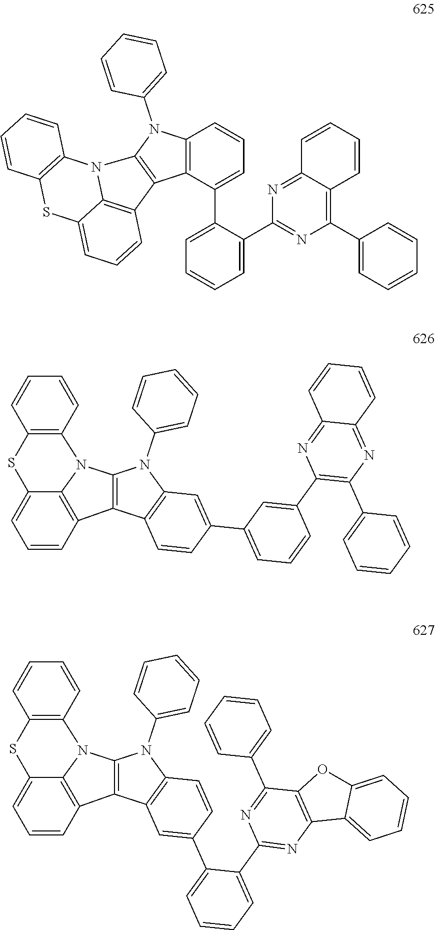 [SMILES: c1ccc(-c2nc(-c3ccccc3-c3cccc4c3c3c5cccc6c5n(c3n4-c3ccccc3)-c3ccccc3S6)nc3ccccc23)cc1 ; c1ccc(-c2nc3ccccc3nc2-c2cccc(-c3ccc4c5c6cccc7c6n(c5n(-c5ccccc5)c4c3)-c3ccccc3S7)c2)cc1 ; c1ccc(-c2nc(-c3ccccc3-c3ccc4c(c3)c3c5cccc6c5n(c3n4-c3ccccc3)-c3ccccc3S6)nc3c2oc2ccccc23)cc1]

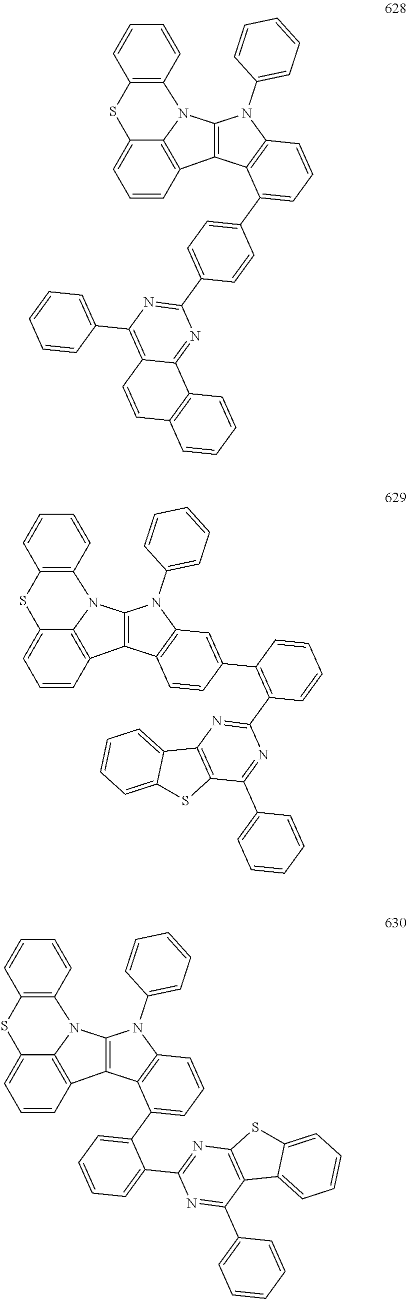 [SMILES: c1ccc(-c2nc(-c3ccc(-c4cccc5c4c4c6cccc7c6n(c4n5-c4ccccc4)-c4ccccc4S7)cc3)nc3c2ccc2ccccc23)cc1 ; c1ccc(-c2nc(-c3ccccc3-c3ccc4c5c6cccc7c6n(c5n(-c5ccccc5)c4c3)-c3ccccc3S7)nc3c2sc2ccccc23)cc1 ; c1ccc(-c2nc(-c3ccccc3-c3cccc4c3c3c5cccc6c5n(c3n4-c3ccccc3)-c3ccccc3S6)nc3sc4ccccc4c23)cc1]

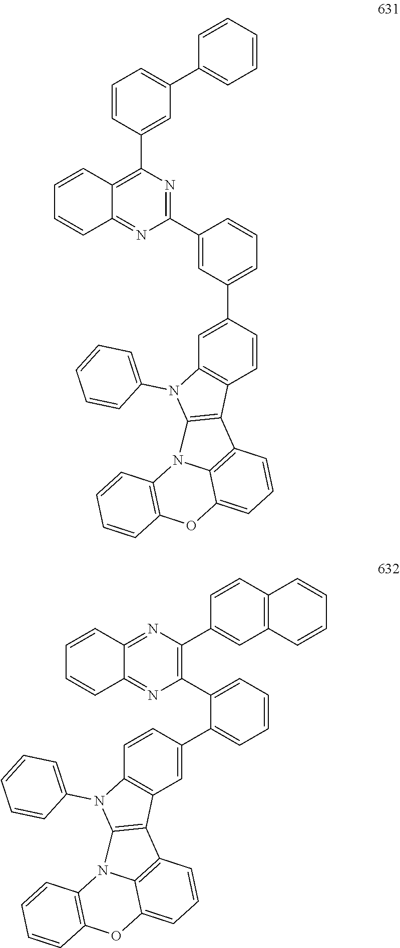 [SMILES: c1ccc(-c2cccc(-c3nc(-c4cccc(-c5ccc6c7c8cccc9c8n(c7n(-c7ccccc7)c6c5)-c5ccccc5O9)c4)nc4ccccc34)c2)cc1 ; c1ccc(-n2c3ccc(-c4ccccc4-c4nc5ccccc5nc4-c4ccc5ccccc5c4)cc3c3c4cccc5c4n(c32)-c2ccccc2O5)cc1]

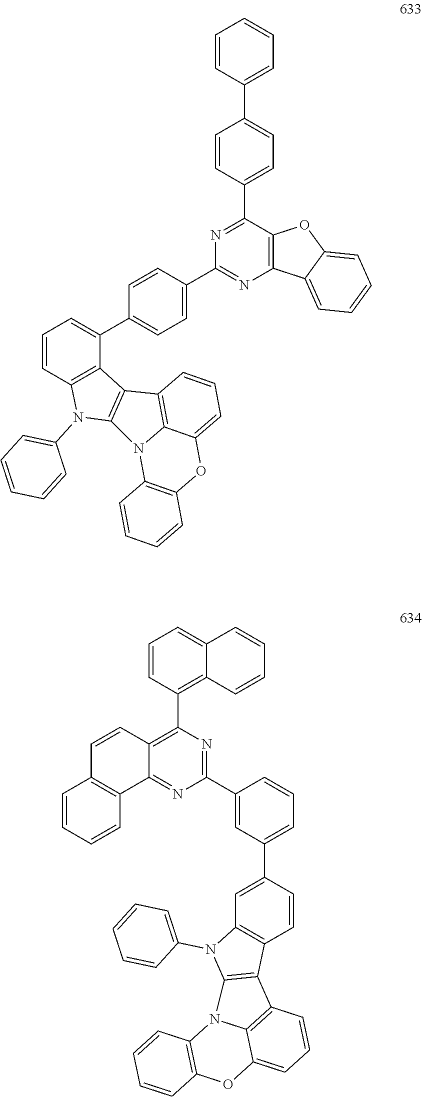 [SMILES: c1ccc(-c2ccc(-c3nc(-c4ccc(-c5cccc6c5c5c7cccc8c7n(c5n6-c5ccccc5)-c5ccccc5O8)cc4)nc4c3oc3ccccc34)cc2)cc1 ; c1ccc(-n2c3cc(-c4cccc(-c5nc(-c6cccc7ccccc67)c6ccc7ccccc7c6n5)c4)ccc3c3c4cccc5c4n(c32)-c2ccccc2O5)cc1]

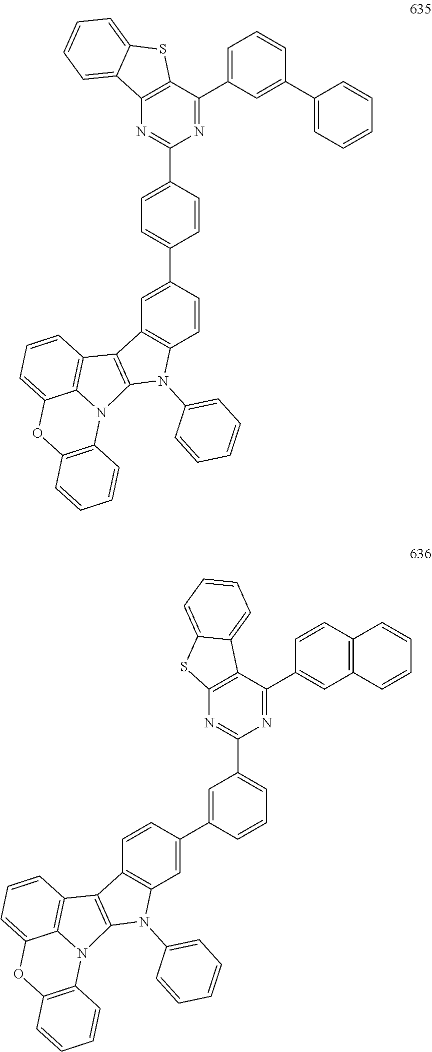 [SMILES: c1ccc(-c2cccc(-c3nc(-c4ccc(-c5ccc6c(c5)c5c7cccc8c7n(c5n6-c5ccccc5)-c5ccccc5O8)cc4)nc4c3sc3ccccc34)c2)cc1 ; c1ccc(-n2c3cc(-c4cccc(-c5nc(-c6ccc7ccccc7c6)c6c(n5)sc5ccccc56)c4)ccc3c3c4cccc5c4n(c32)-c2ccccc2O5)cc1]

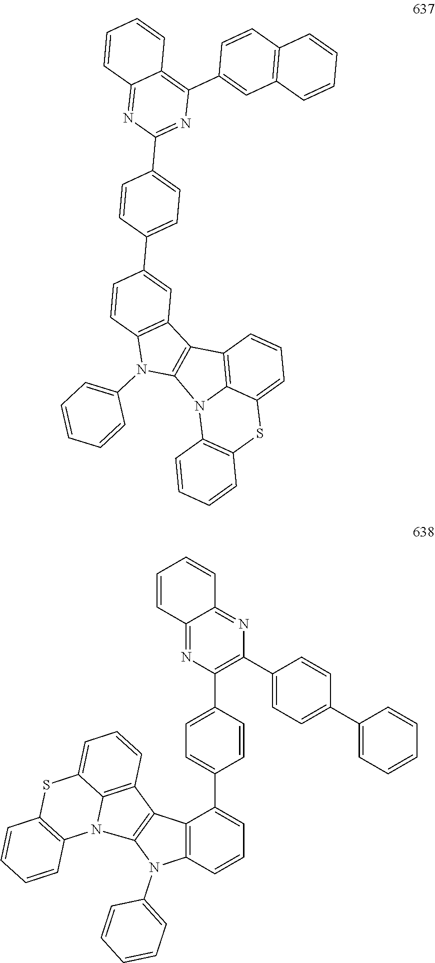 [SMILES: c1ccc(-n2c3ccc(-c4ccc(-c5nc(-c6ccc7ccccc7c6)c6ccccc6n5)cc4)cc3c3c4cccc5c4n(c32)-c2ccccc2S5)cc1 ; c1ccc(-c2ccc(-c3nc4ccccc4nc3-c3ccc(-c4cccc5c4c4c6cccc7c6n(c4n5-c4ccccc4)-c4ccccc4S7)cc3)cc2)cc1]

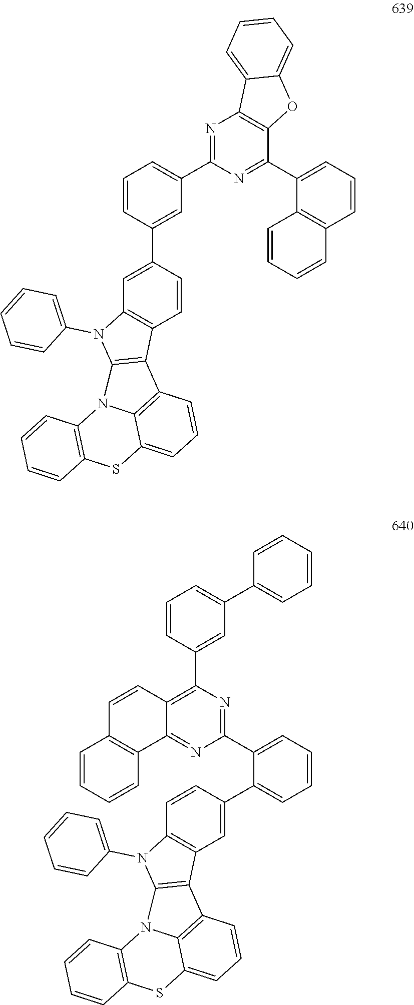 [SMILES: c1ccc(-n2c3cc(-c4cccc(-c5nc(-c6cccc7ccccc67)c6oc7ccccc7c6n5)c4)ccc3c3c4cccc5c4n(c32)-c2ccccc2S5)cc1 ; c1ccc(-c2cccc(-c3nc(-c4ccccc4-c4ccc5c(c4)c4c6cccc7c6n(c4n5-c4ccccc4)-c4ccccc4S7)nc4c3ccc3ccccc34)c2)cc1]

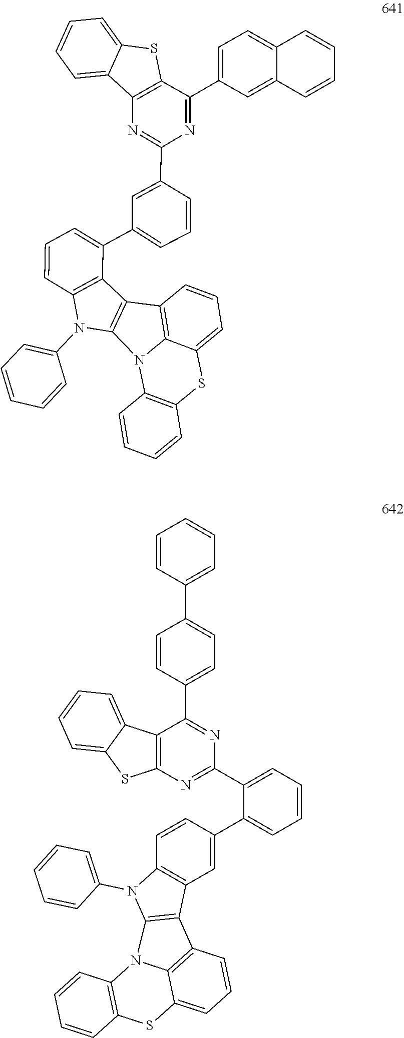 [SMILES: c1ccc(-n2c3cccc(-c4cccc(-c5nc(-c6ccc7ccccc7c6)c6sc7ccccc7c6n5)c4)c3c3c4cccc5c4n(c32)-c2ccccc2S5)cc1 ; c1ccc(-c2ccc(-c3nc(-c4ccccc4-c4ccc5c(c4)c4c6cccc7c6n(c4n5-c4ccccc4)-c4ccccc4S7)nc4sc5ccccc5c34)cc2)cc1]

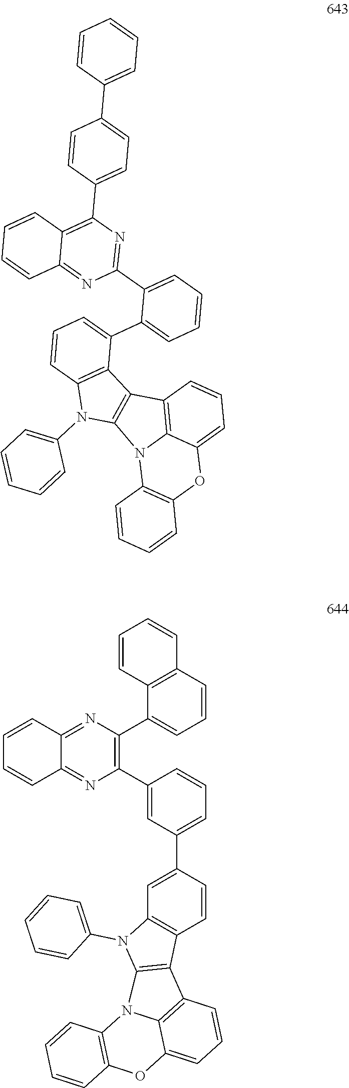 [SMILES: c1ccc(-c2ccc(-c3nc(-c4ccccc4-c4cccc5c4c4c6cccc7c6n(c4n5-c4ccccc4)-c4ccccc4O7)nc4ccccc34)cc2)cc1 ; c1ccc(-n2c3cc(-c4cccc(-c5nc6ccccc6nc5-c5cccc6ccccc56)c4)ccc3c3c4cccc5c4n(c32)-c2ccccc2O5)cc1]

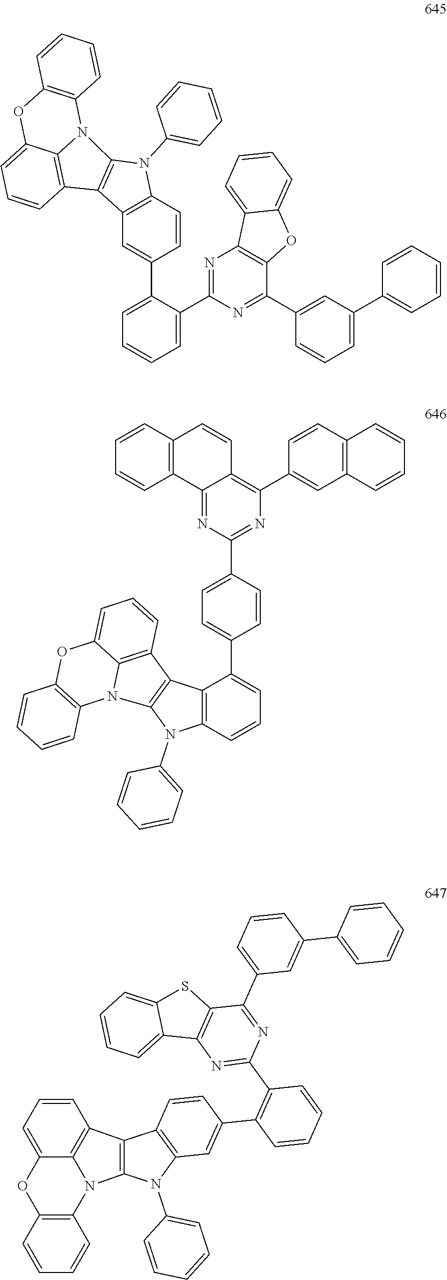 [SMILES: c1ccc(-c2cccc(-c3nc(-c4ccccc4-c4ccc5c(c4)c4c6cccc7c6n(c4n5-c4ccccc4)-c4ccccc4O7)nc4c3oc3ccccc34)c2)cc1 ; c1ccc(-n2c3cccc(-c4ccc(-c5nc(-c6ccc7ccccc7c6)c6ccc7ccccc7c6n5)cc4)c3c3c4cccc5c4n(c32)-c2ccccc2O5)cc1 ; c1ccc(-c2cccc(-c3nc(-c4ccccc4-c4ccc5c6c7cccc8c7n(c6n(-c6ccccc6)c5c4)-c4ccccc4O8)nc4c3sc3ccccc34)c2)cc1]

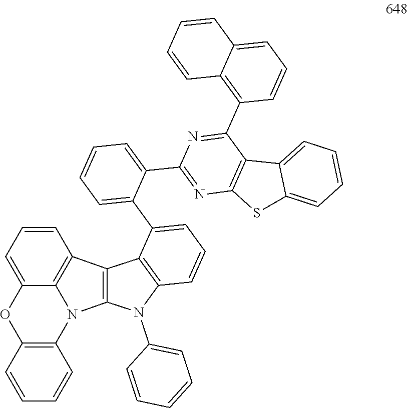 [SMILES: c1ccc(-n2c3cccc(-c4ccccc4-c4nc(-c5cccc6ccccc56)c5c(n4)sc4ccccc45)c3c3c4cccc5c4n(c32)-c2ccccc2O5)cc1]

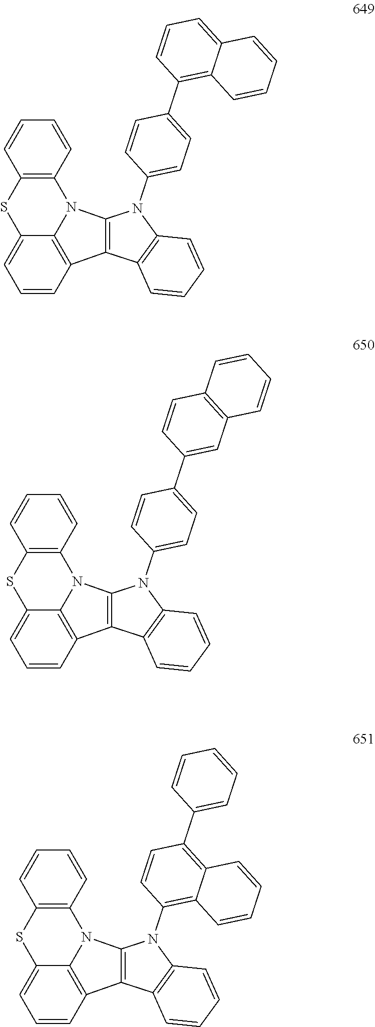 [SMILES: c1ccc2c(c1)Sc1cccc3c4c5ccccc5n(-c5ccc(-c6cccc7ccccc67)cc5)c4n-2c13 ; c1ccc2c(c1)Sc1cccc3c4c5ccccc5n(-c5ccc(-c6ccc7ccccc7c6)cc5)c4n-2c13 ; c1ccc(-c2ccc(-n3c4ccccc4c4c5cccc6c5n(c43)-c3ccccc3S6)c3ccccc23)cc1]

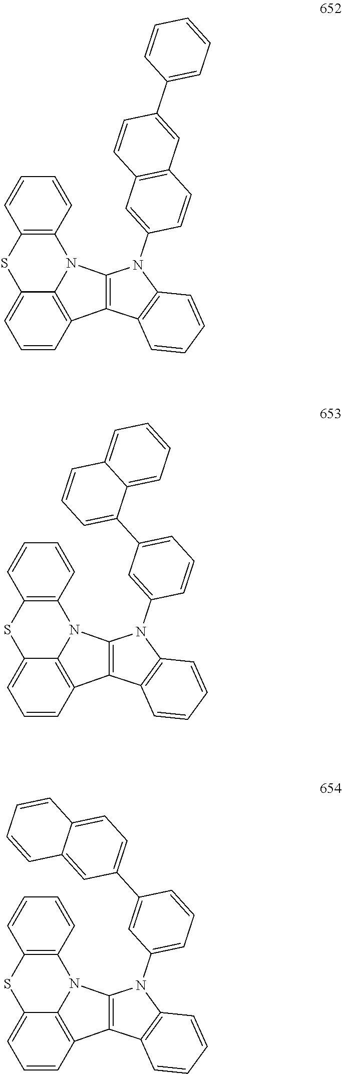 [SMILES: c1ccc(-c2ccc3cc(-n4c5ccccc5c5c6cccc7c6n(c54)-c4ccccc4S7)ccc3c2)cc1 ; c1cc(-c2cccc3ccccc23)cc(-n2c3ccccc3c3c4cccc5c4n(c32)-c2ccccc2S5)c1 ; c1cc(-c2ccc3ccccc3c2)cc(-n2c3ccccc3c3c4cccc5c4n(c32)-c2ccccc2S5)c1]

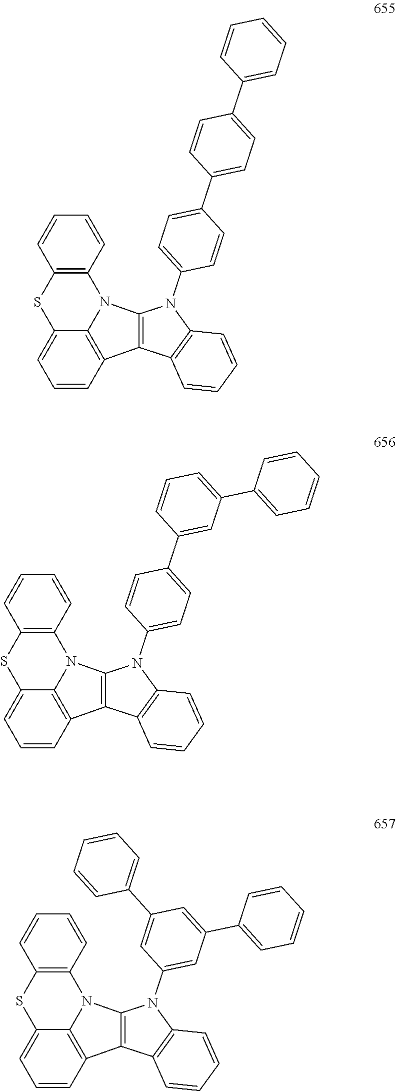 [SMILES: c1ccc(-c2ccc(-c3ccc(-n4c5ccccc5c5c6cccc7c6n(c54)-c4ccccc4S7)cc3)cc2)cc1 ; c1ccc(-c2cccc(-c3ccc(-n4c5ccccc5c5c6cccc7c6n(c54)-c4ccccc4S7)cc3)c2)cc1 ; c1ccc(-c2cc(-c3ccccc3)cc(-n3c4ccccc4c4c5cccc6c5n(c43)-c3ccccc3S6)c2)cc1]

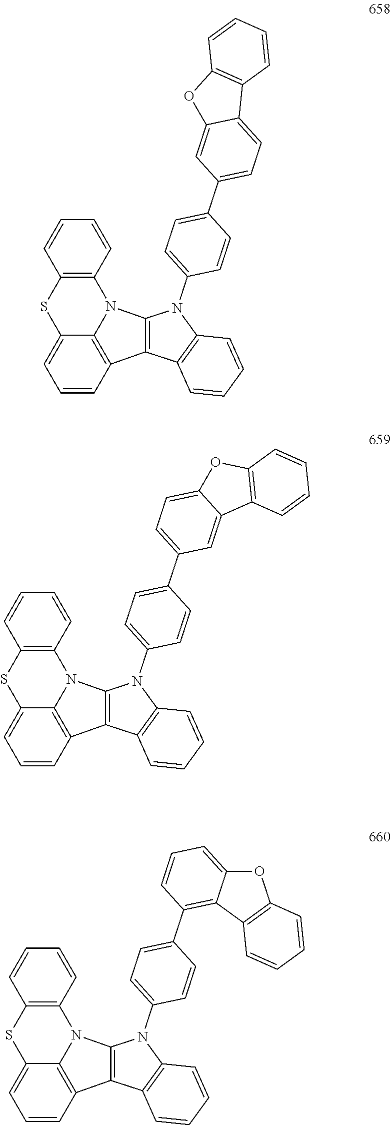 [SMILES: c1ccc2c(c1)Sc1cccc3c4c5ccccc5n(-c5ccc(-c6ccc7c(c6)oc6ccccc67)cc5)c4n-2c13 ; c1ccc2c(c1)Sc1cccc3c4c5ccccc5n(-c5ccc(-c6ccc7oc8ccccc8c7c6)cc5)c4n-2c13 ; c1ccc2c(c1)Sc1cccc3c4c5ccccc5n(-c5ccc(-c6cccc7oc8ccccc8c67)cc5)c4n-2c13]

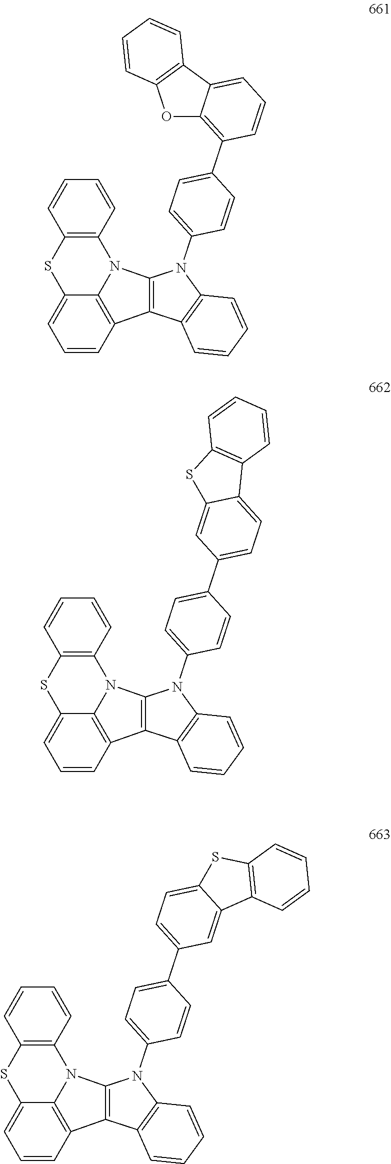

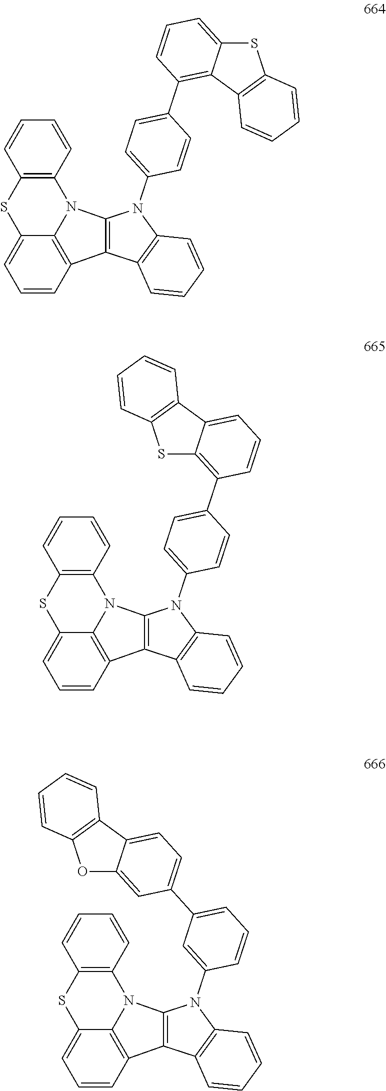 [SMILES: c1ccc2c(c1)Sc1cccc3c4c5ccccc5n(-c5ccc(-c6cccc7sc8ccccc8c67)cc5)c4n-2c13 ; c1ccc2c(c1)Sc1cccc3c4c5ccccc5n(-c5ccc(-c6cccc7c6sc6ccccc67)cc5)c4n-2c13 ; c1cc(-c2ccc3c(c2)oc2ccccc23)cc(-n2c3ccccc3c3c4cccc5c4n(c32)-c2ccccc2S5)c1]

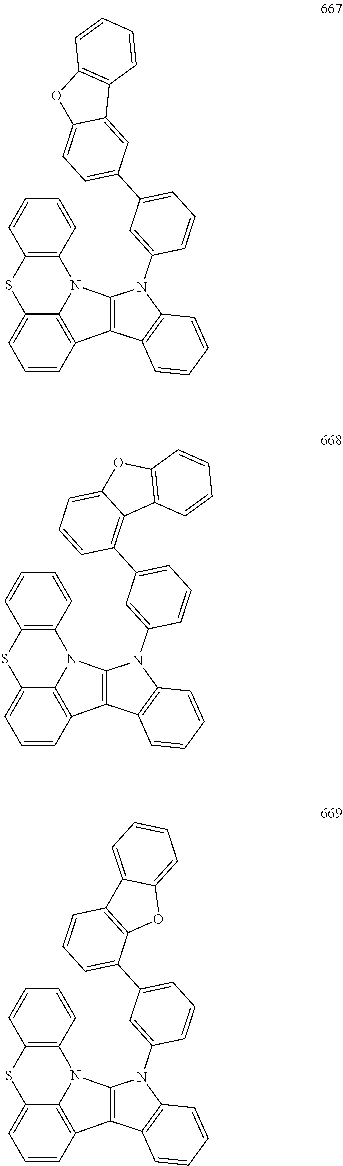 [SMILES: c1cc(-c2ccc3oc4ccccc4c3c2)cc(-n2c3ccccc3c3c4cccc5c4n(c32)-c2ccccc2S5)c1 ; c1cc(-c2cccc3oc4ccccc4c23)cc(-n2c3ccccc3c3c4cccc5c4n(c32)-c2ccccc2S5)c1 ; c1cc(-c2cccc3c2oc2ccccc23)cc(-n2c3ccccc3c3c4cccc5c4n(c32)-c2ccccc2S5)c1]

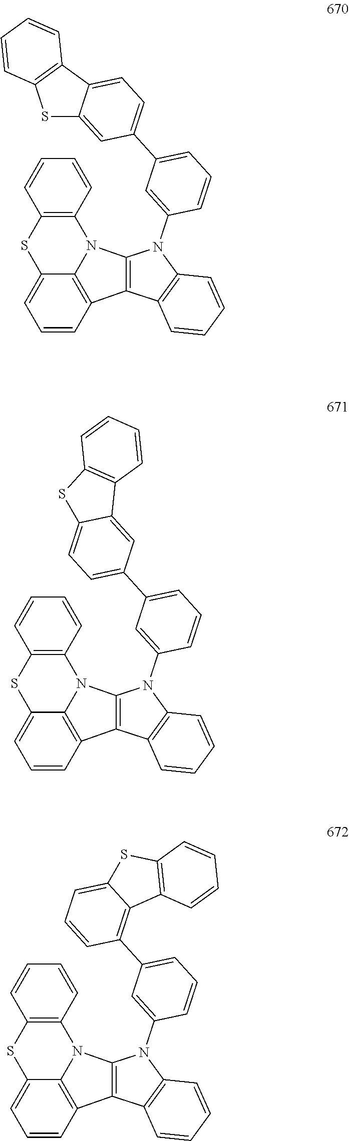 [SMILES: c1cc(-c2ccc3c(c2)sc2ccccc23)cc(-n2c3ccccc3c3c4cccc5c4n(c32)-c2ccccc2S5)c1 ; c1cc(-c2ccc3sc4ccccc4c3c2)cc(-n2c3ccccc3c3c4cccc5c4n(c32)-c2ccccc2S5)c1 ; c1cc(-c2cccc3sc4ccccc4c23)cc(-n2c3ccccc3c3c4cccc5c4n(c32)-c2ccccc2S5)c1]

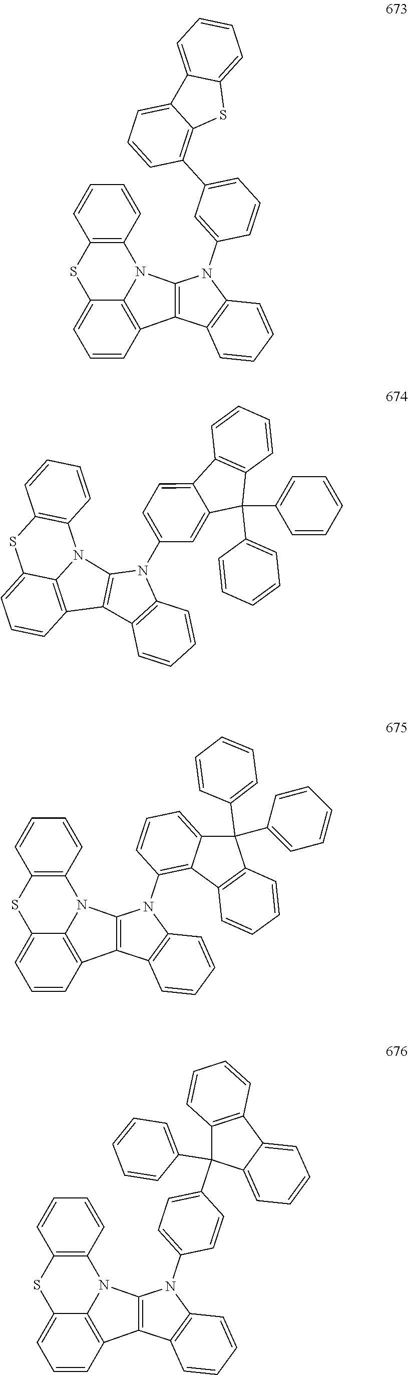 [SMILES: c1cc(-c2cccc3c2sc2ccccc23)cc(-n2c3ccccc3c3c4cccc5c4n(c32)-c2ccccc2S5)c1 ; c1ccc(C2(c3ccccc3)c3ccccc3-c3ccc(-n4c5ccccc5c5c6cccc7c6n(c54)-c4ccccc4S7)cc32)cc1 ; c1ccc(C2(c3ccccc3)c3ccccc3-c3c(-n4c5ccccc5c5c6cccc7c6n(c54)-c4ccccc4S7)cccc32)cc1 ; c1ccc(C2(c3ccc(-n4c5ccccc5c5c6cccc7c6n(c54)-c4ccccc4S7)cc3)c3ccccc3-c3ccccc32)cc1]

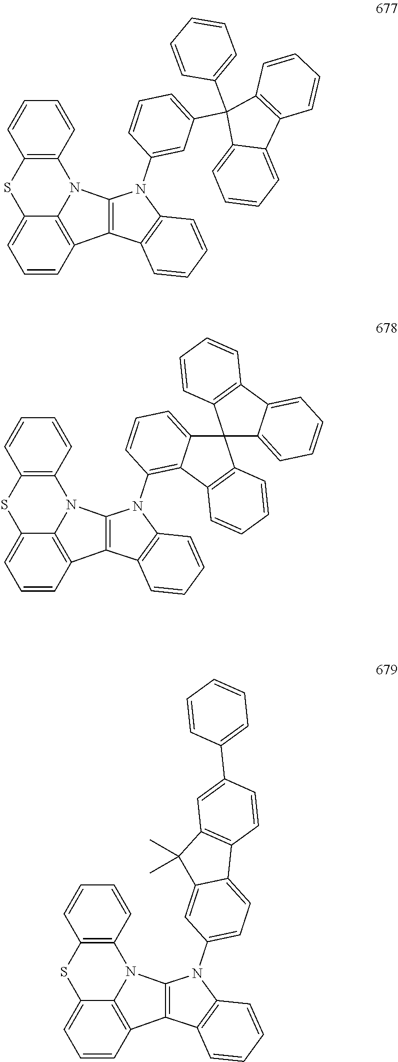 [SMILES: c1ccc(C2(c3cccc(-n4c5ccccc5c5c6cccc7c6n(c54)-c4ccccc4S7)c3)c3ccccc3-c3ccccc32)cc1 ; c1ccc2c(c1)Sc1cccc3c4c5ccccc5n(-c5cccc6c5-c5ccccc5C65c6ccccc6-c6ccccc65)c4n-2c13 ; CC1(C)c2cc(-c3ccccc3)ccc2-c2ccc(-n3c4ccccc4c4c5cccc6c5n(c43)-c3ccccc3S6)cc21]

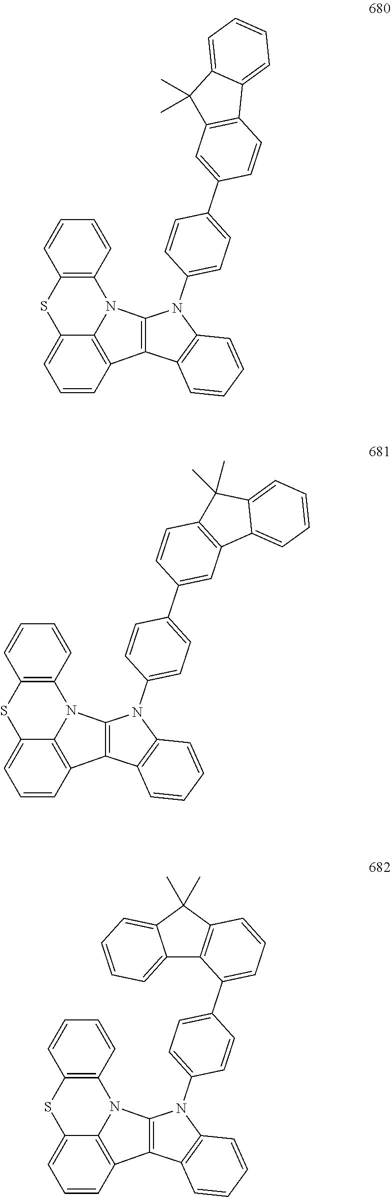 [SMILES: CC1(C)c2ccccc2-c2ccc(-c3ccc(-n4c5ccccc5c5c6cccc7c6n(c54)-c4ccccc4S7)cc3)cc21 ; CC1(C)c2ccccc2-c2cc(-c3ccc(-n4c5ccccc5c5c6cccc7c6n(c54)-c4ccccc4S7)cc3)ccc21 ; CC1(C)c2ccccc2-c2c(-c3ccc(-n4c5ccccc5c5c6cccc7c6n(c54)-c4ccccc4S7)cc3)cccc21]

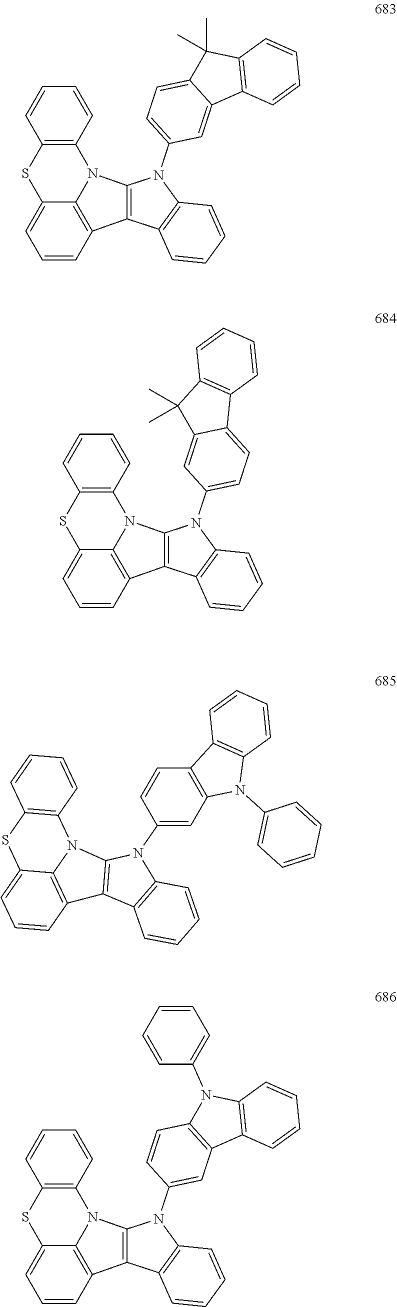 [SMILES: CC1(C)c2ccccc2-c2cc(-n3c4ccccc4c4c5cccc6c5n(c43)-c3ccccc3S6)ccc21 ; CC1(C)c2ccccc2-c2ccc(-n3c4ccccc4c4c5cccc6c5n(c43)-c3ccccc3S6)cc21 ; c1ccc(-n2c3ccccc3c3ccc(-n4c5ccccc5c5c6cccc7c6n(c54)-c4ccccc4S7)cc32)cc1 ; c1ccc(-n2c3ccccc3c3cc(-n4c5ccccc5c5c6cccc7c6n(c54)-c4ccccc4S7)ccc32)cc1]

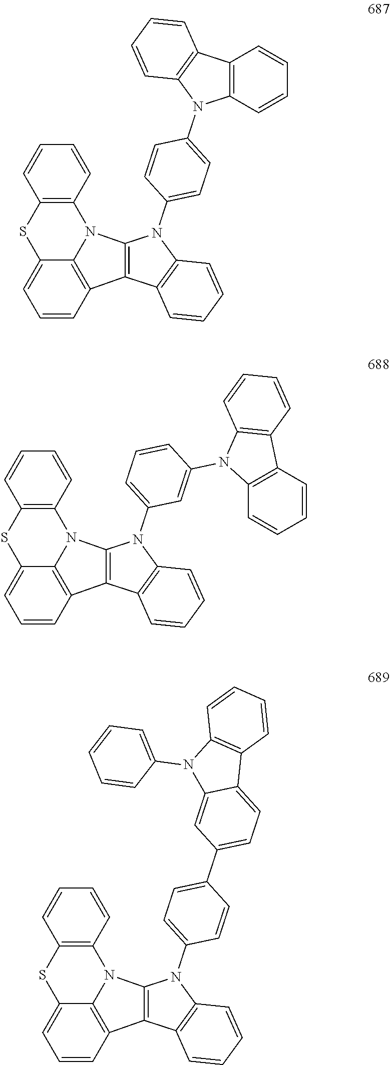 [SMILES: c1ccc2c(c1)Sc1cccc3c4c5ccccc5n(-c5ccc(-n6c7ccccc7c7ccccc76)cc5)c4n-2c13 ; c1cc(-n2c3ccccc3c3ccccc32)cc(-n2c3ccccc3c3c4cccc5c4n(c32)-c2ccccc2S5)c1 ; c1ccc(-n2c3ccccc3c3ccc(-c4ccc(-n5c6ccccc6c6c7cccc8c7n(c65)-c5ccccc5S8)cc4)cc32)cc1]

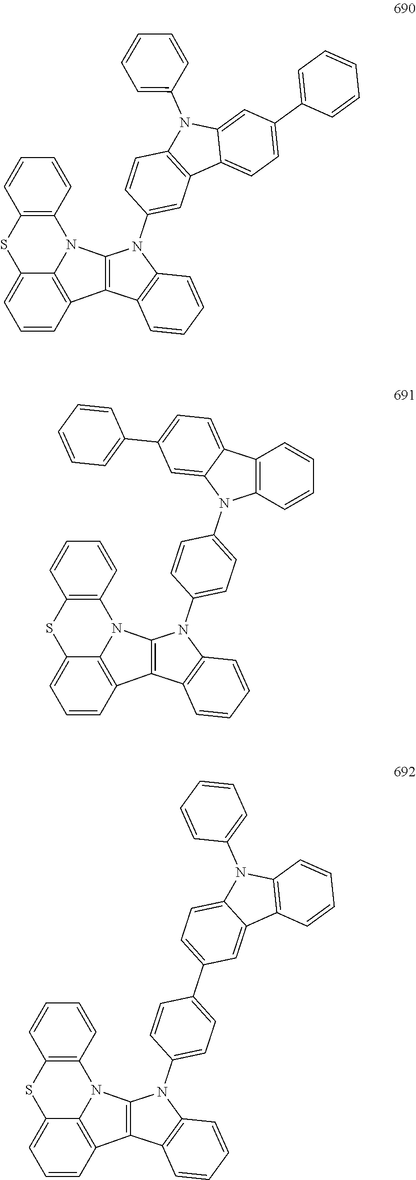 [SMILES: c1ccc(-c2ccc3c4cc(-n5c6ccccc6c6c7cccc8c7n(c65)-c5ccccc5S8)ccc4n(-c4ccccc4)c3c2)cc1 ; c1ccc(-c2ccc3c4ccccc4n(-c4ccc(-n5c6ccccc6c6c7cccc8c7n(c65)-c5ccccc5S8)cc4)c3c2)cc1 ; c1ccc(-n2c3ccccc3c3cc(-c4ccc(-n5c6ccccc6c6c7cccc8c7n(c65)-c5ccccc5S8)cc4)ccc32)cc1]

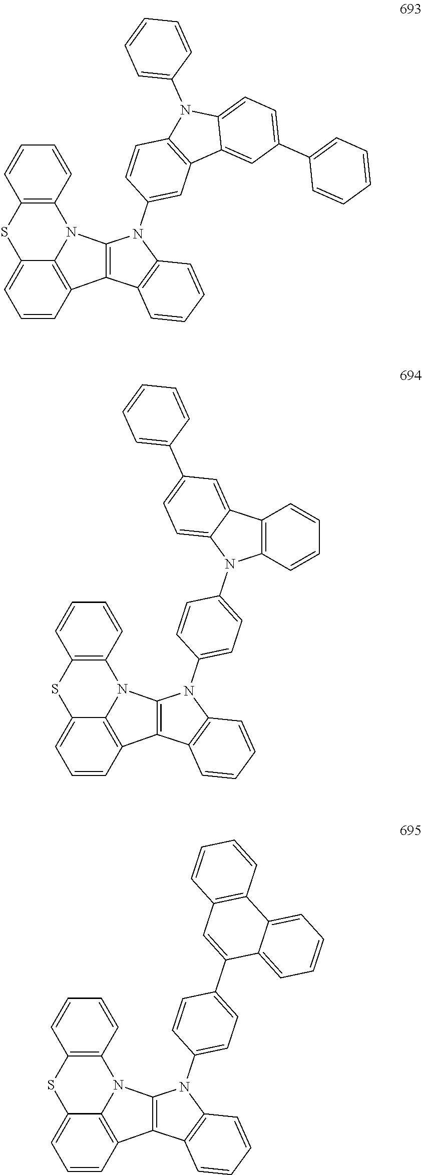 [SMILES: c1ccc(-c2ccc3c(c2)c2cc(-n4c5ccccc5c5c6cccc7c6n(c54)-c4ccccc4S7)ccc2n3-c2ccccc2)cc1 ; c1ccc(-c2ccc3c(c2)c2ccccc2n3-c2ccc(-n3c4ccccc4c4c5cccc6c5n(c43)-c3ccccc3S6)cc2)cc1 ; c1ccc2c(c1)Sc1cccc3c4c5ccccc5n(-c5ccc(-c6cc7ccccc7c7ccccc67)cc5)c4n-2c13]

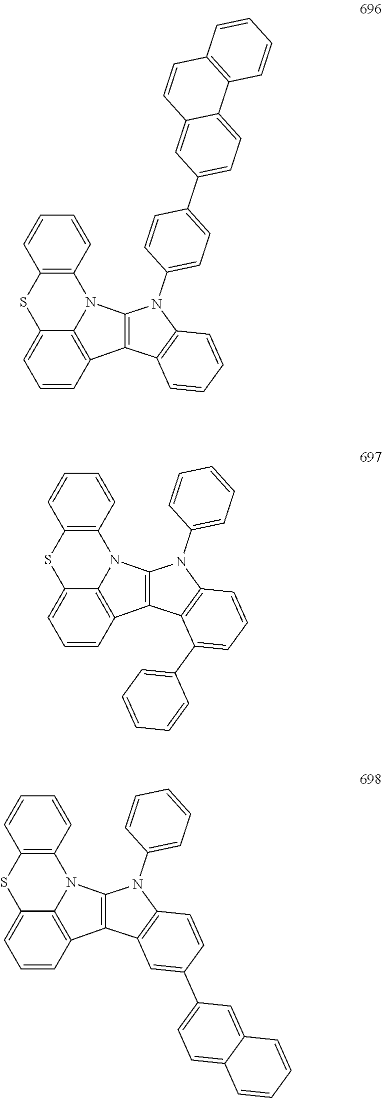 [SMILES: c1ccc2c(c1)Sc1cccc3c4c5ccccc5n(-c5ccc(-c6ccc7c(ccc8ccccc87)c6)cc5)c4n-2c13 ; c1ccc(-c2cccc3c2c2c4cccc5c4n(c2n3-c2ccccc2)-c2ccccc2S5)cc1 ; c1ccc(-n2c3ccc(-c4ccc5ccccc5c4)cc3c3c4cccc5c4n(c32)-c2ccccc2S5)cc1]

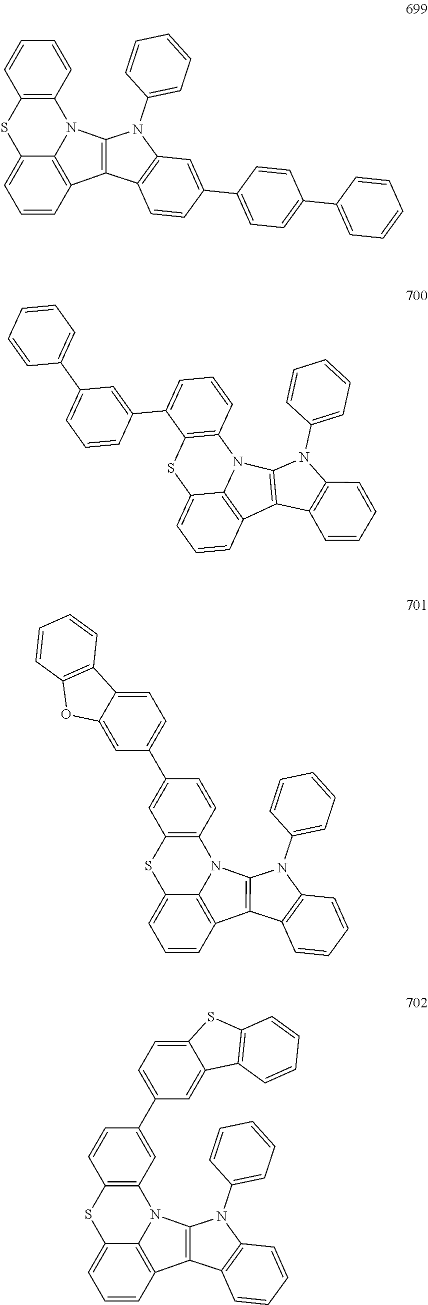 [SMILES: c1ccc(-c2ccc(-c3ccc4c5c6cccc7c6n(c5n(-c5ccccc5)c4c3)-c3ccccc3S7)cc2)cc1 ; c1ccc(-c2cccc(-c3cccc4c3Sc3cccc5c6c7ccccc7n(-c7ccccc7)c6n-4c35)c2)cc1 ; c1ccc(-n2c3ccccc3c3c4cccc5c4n(c32)-c2ccc(-c3ccc4c(c3)oc3ccccc34)cc2S5)cc1 ; c1ccc(-n2c3ccccc3c3c4cccc5c4n(c32)-c2cc(-c3ccc4sc6ccccc6c4c3)ccc2S5)cc1]

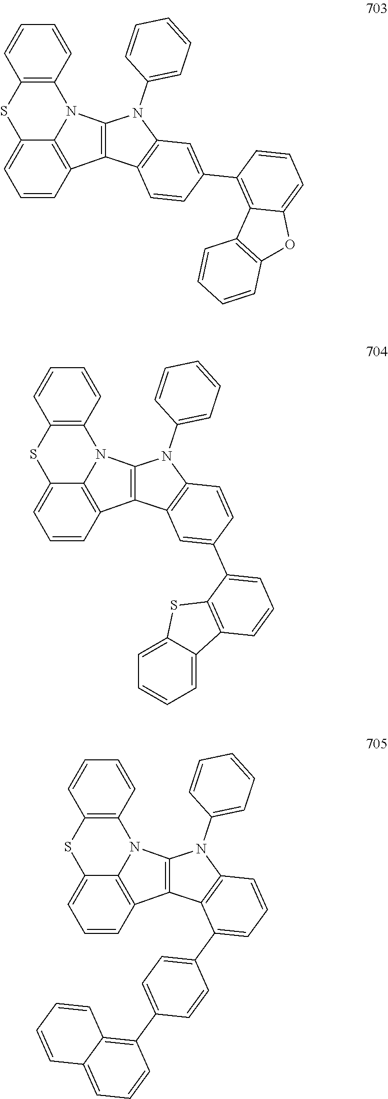 [SMILES: c1ccc(-n2c3cc(-c4cccc5oc6ccccc6c45)ccc3c3c4cccc5c4n(c32)-c2ccccc2S5)cc1 ; c1ccc(-n2c3ccc(-c4cccc5c4sc4ccccc45)cc3c3c4cccc5c4n(c32)-c2ccccc2S5)cc1 ; c1ccc(-n2c3cccc(-c4ccc(-c5cccc6ccccc56)cc4)c3c3c4cccc5c4n(c32)-c2ccccc2S5)cc1]

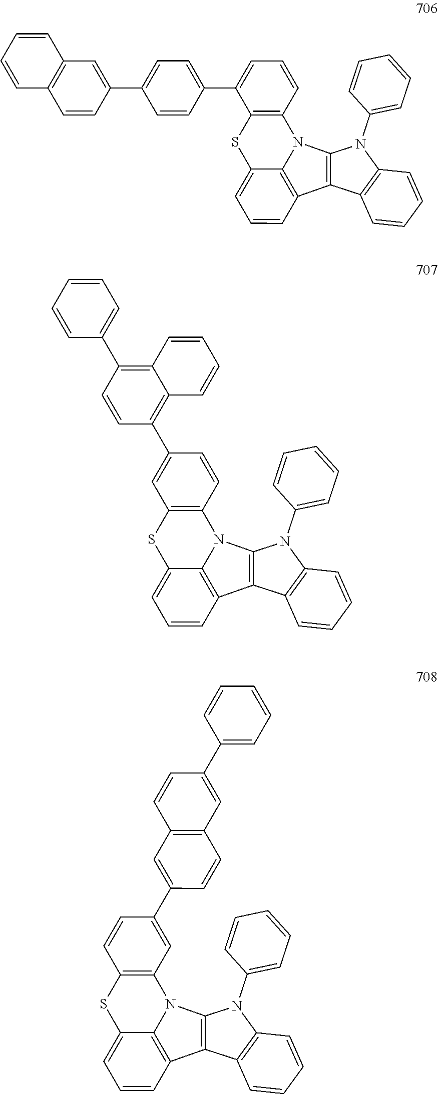 [SMILES: c1ccc(-n2c3ccccc3c3c4cccc5c4n(c32)-c2cccc(-c3ccc(-c4ccc6ccccc6c4)cc3)c2S5)cc1 ; c1ccc(-c2ccc(-c3ccc4c(c3)Sc3cccc5c6c7ccccc7n(-c7ccccc7)c6n-4c35)c3ccccc23)cc1 ; c1ccc(-c2ccc3cc(-c4ccc5c(c4)-n4c6c(cccc6c6c7ccccc7n(-c7ccccc7)c64)S5)ccc3c2)cc1]

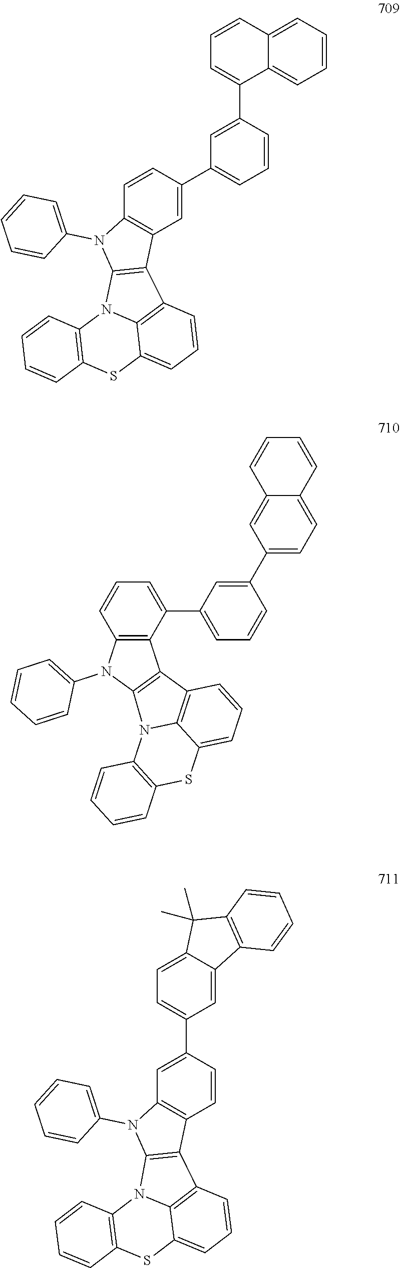 [SMILES: c1ccc(-n2c3ccc(-c4cccc(-c5cccc6ccccc56)c4)cc3c3c4cccc5c4n(c32)-c2ccccc2S5)cc1 ; c1ccc(-n2c3cccc(-c4cccc(-c5ccc6ccccc6c5)c4)c3c3c4cccc5c4n(c32)-c2ccccc2S5)cc1 ; CC1(C)c2ccccc2-c2cc(-c3ccc4c5c6cccc7c6n(c5n(-c5ccccc5)c4c3)-c3ccccc3S7)ccc21]

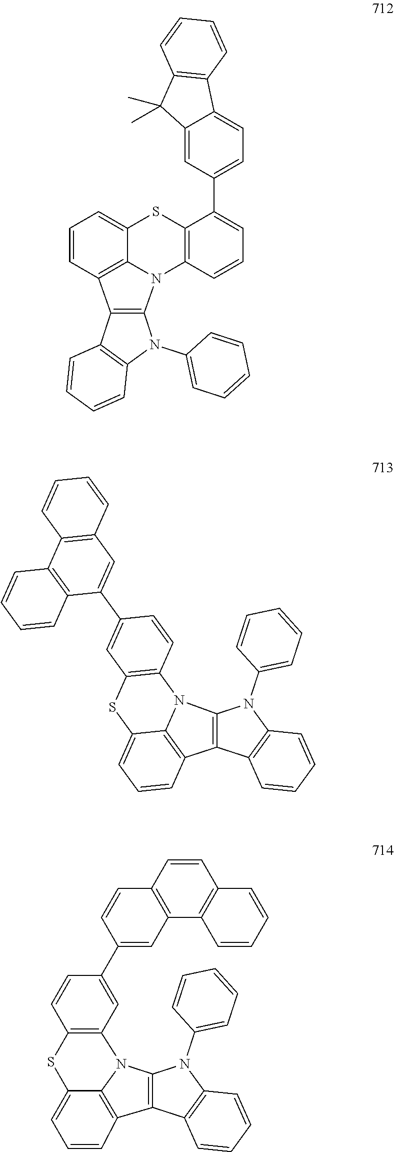 [SMILES: CC1(C)c2ccccc2-c2ccc(-c3cccc4c3Sc3cccc5c6c7ccccc7n(-c7ccccc7)c6n-4c35)cc21 ; c1ccc(-n2c3ccccc3c3c4cccc5c4n(c32)-c2ccc(-c3cc4ccccc4c4ccccc34)cc2S5)cc1 ; c1ccc(-n2c3ccccc3c3c4cccc5c4n(c32)-c2cc(-c3ccc4ccc6ccccc6c4c3)ccc2S5)cc1]

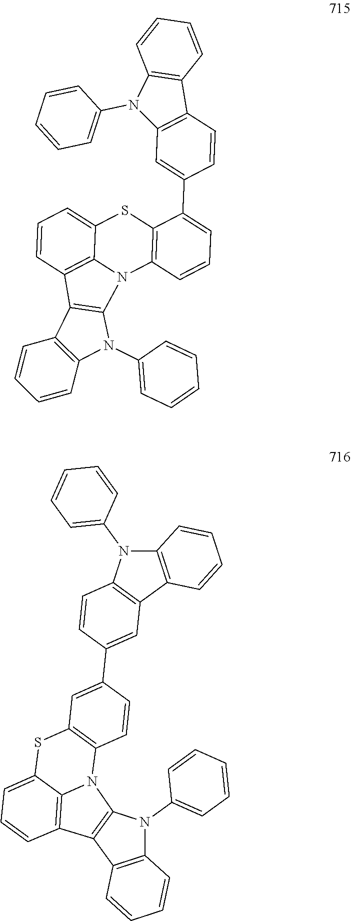 [SMILES: c1ccc(-n2c3ccccc3c3ccc(-c4cccc5c4Sc4cccc6c7c8ccccc8n(-c8ccccc8)c7n-5c46)cc32)cc1 ; c1ccc(-n2c3ccccc3c3cc(-c4ccc5c(c4)Sc4cccc6c7c8ccccc8n(-c8ccccc8)c7n-5c46)ccc32)cc1]

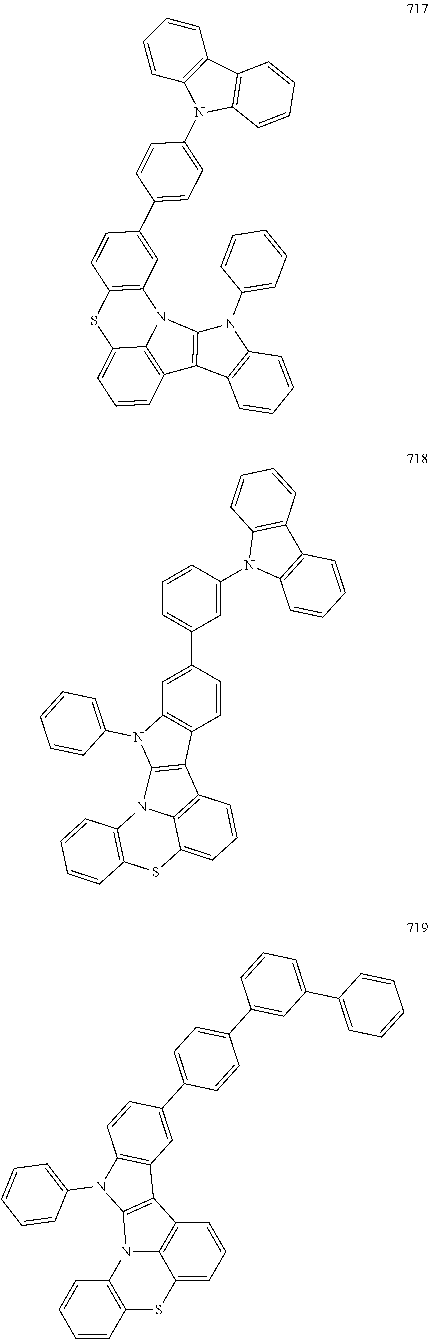 [SMILES: c1ccc(-n2c3ccccc3c3c4cccc5c4n(c32)-c2cc(-c3ccc(-n4c6ccccc6c6ccccc64)cc3)ccc2S5)cc1 ; c1ccc(-n2c3cc(-c4cccc(-n5c6ccccc6c6ccccc65)c4)ccc3c3c4cccc5c4n(c32)-c2ccccc2S5)cc1 ; c1ccc(-c2cccc(-c3ccc(-c4ccc5c(c4)c4c6cccc7c6n(c4n5-c4ccccc4)-c4ccccc4S7)cc3)c2)cc1]

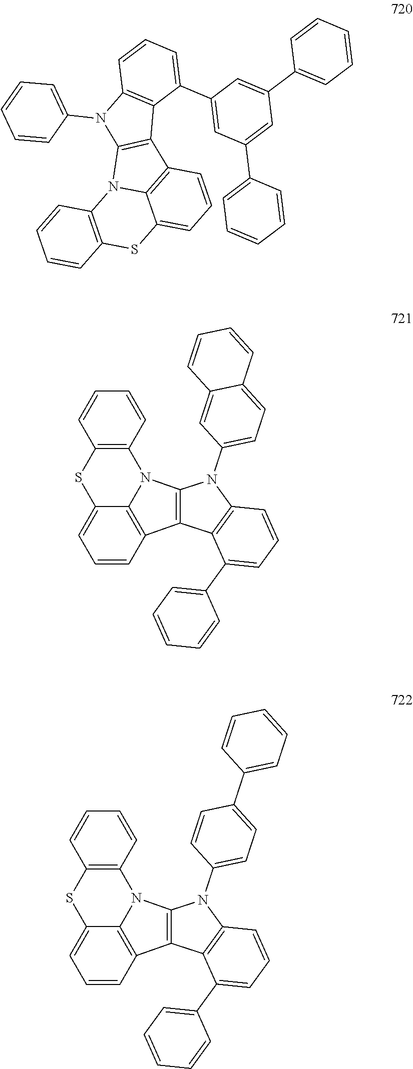 [SMILES: c1ccc(-c2cc(-c3ccccc3)cc(-c3cccc4c3c3c5cccc6c5n(c3n4-c3ccccc3)-c3ccccc3S6)c2)cc1 ; c1ccc(-c2cccc3c2c2c4cccc5c4n(c2n3-c2ccc3ccccc3c2)-c2ccccc2S5)cc1 ; c1ccc(-c2ccc(-n3c4cccc(-c5ccccc5)c4c4c5cccc6c5n(c43)-c3ccccc3S6)cc2)cc1]

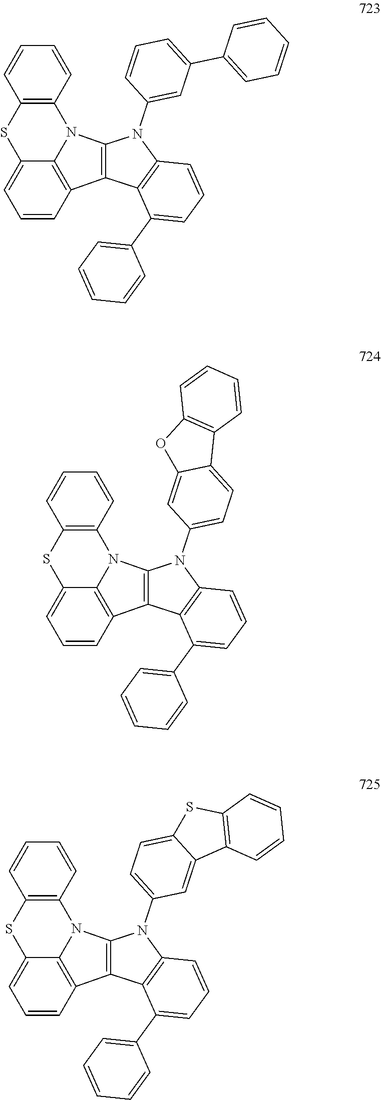 [SMILES: c1ccc(-c2cccc(-n3c4cccc(-c5ccccc5)c4c4c5cccc6c5n(c43)-c3ccccc3S6)c2)cc1 ; c1ccc(-c2cccc3c2c2c4cccc5c4n(c2n3-c2ccc3c(c2)oc2ccccc23)-c2ccccc2S5)cc1 ; c1ccc(-c2cccc3c2c2c4cccc5c4n(c2n3-c2ccc3sc4ccccc4c3c2)-c2ccccc2S5)cc1]

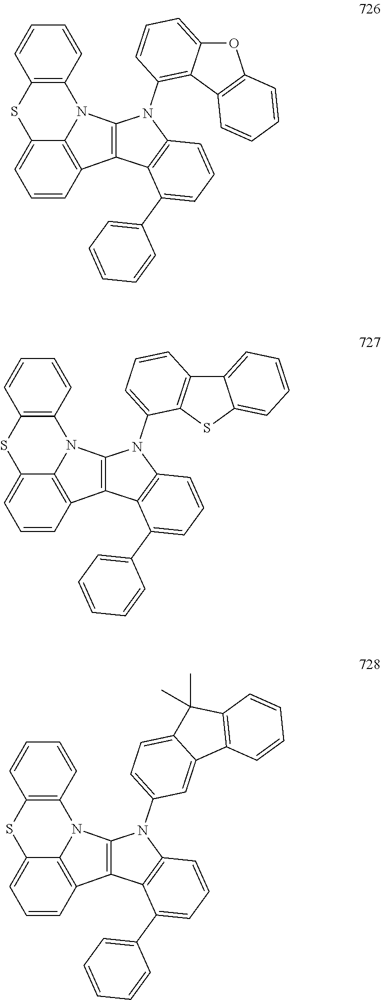 [SMILES: c1ccc(-c2cccc3c2c2c4cccc5c4n(c2n3-c2cccc3oc4ccccc4c23)-c2ccccc2S5)cc1 ; c1ccc(-c2cccc3c2c2c4cccc5c4n(c2n3-c2cccc3c2sc2ccccc23)-c2ccccc2S5)cc1 ; CC1(C)c2ccccc2-c2cc(-n3c4cccc(-c5ccccc5)c4c4c5cccc6c5n(c43)-c3ccccc3S6)ccc21]

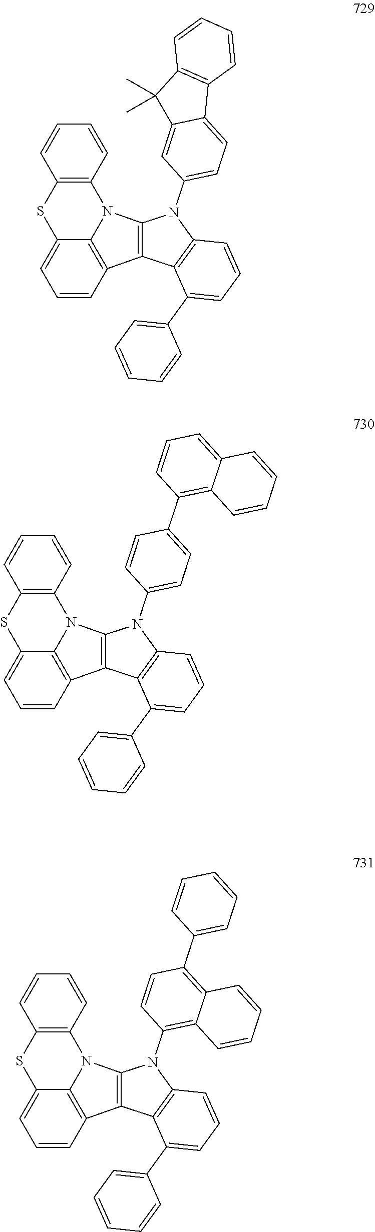 [SMILES: CC1(C)c2ccccc2-c2ccc(-n3c4cccc(-c5ccccc5)c4c4c5cccc6c5n(c43)-c3ccccc3S6)cc21 ; c1ccc(-c2cccc3c2c2c4cccc5c4n(c2n3-c2ccc(-c3cccc4ccccc34)cc2)-c2ccccc2S5)cc1 ; c1ccc(-c2ccc(-n3c4cccc(-c5ccccc5)c4c4c5cccc6c5n(c43)-c3ccccc3S6)c3ccccc23)cc1]

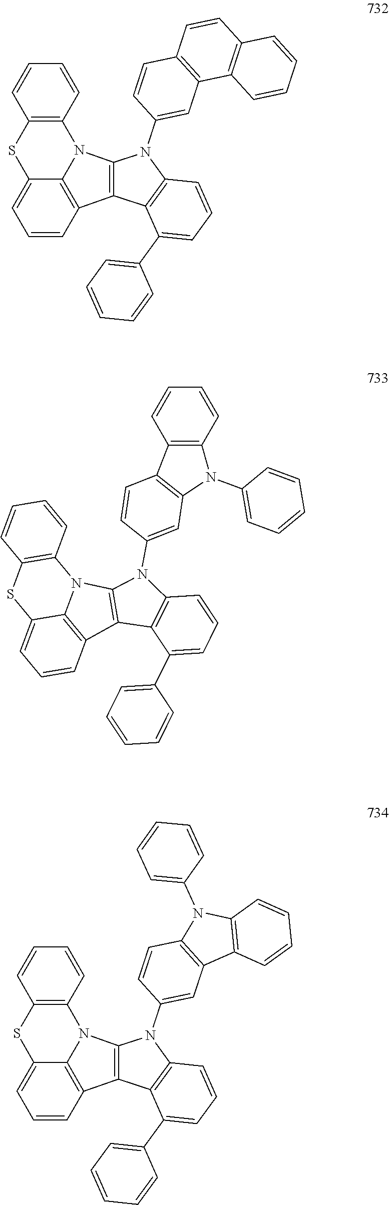 [SMILES: c1ccc(-c2cccc3c2c2c4cccc5c4n(c2n3-c2ccc3ccc4ccccc4c3c2)-c2ccccc2S5)cc1 ; c1ccc(-c2cccc3c2c2c4cccc5c4n(c2n3-c2ccc3c4ccccc4n(-c4ccccc4)c3c2)-c2ccccc2S5)cc1 ; c1ccc(-c2cccc3c2c2c4cccc5c4n(c2n3-c2ccc3c(c2)c2ccccc2n3-c2ccccc2)-c2ccccc2S5)cc1]

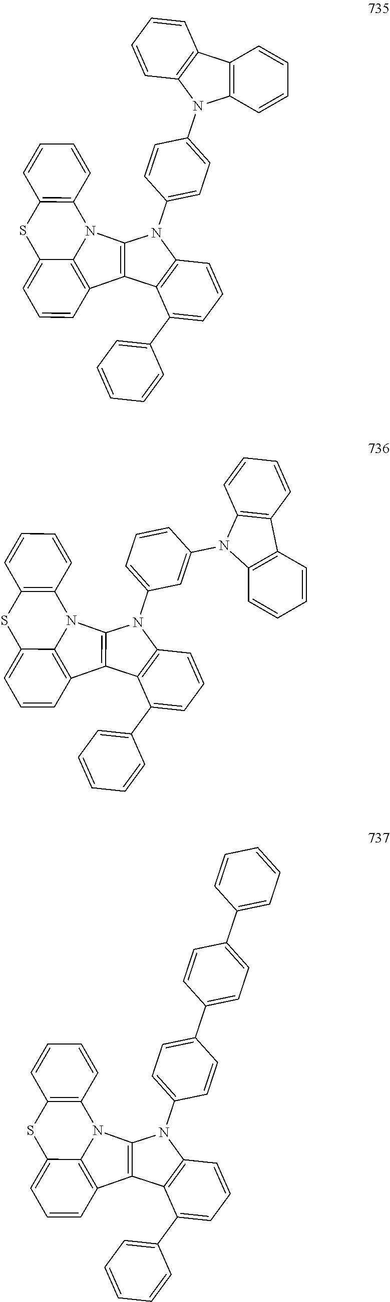 [SMILES: c1ccc(-c2cccc3c2c2c4cccc5c4n(c2n3-c2ccc(-n3c4ccccc4c4ccccc43)cc2)-c2ccccc2S5)cc1 ; c1ccc(-c2cccc3c2c2c4cccc5c4n(c2n3-c2cccc(-n3c4ccccc4c4ccccc43)c2)-c2ccccc2S5)cc1 ; c1ccc(-c2ccc(-c3ccc(-n4c5cccc(-c6ccccc6)c5c5c6cccc7c6n(c54)-c4ccccc4S7)cc3)cc2)cc1]

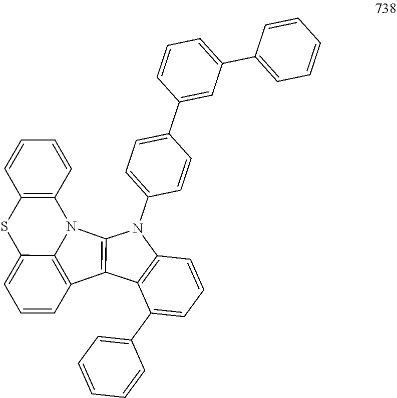 [SMILES: c1ccc(-c2cccc(-c3ccc(-n4c5cccc(-c6ccccc6)c5c5c6cccc7c6n(c54)-c4ccccc4S7)cc3)c2)cc1]

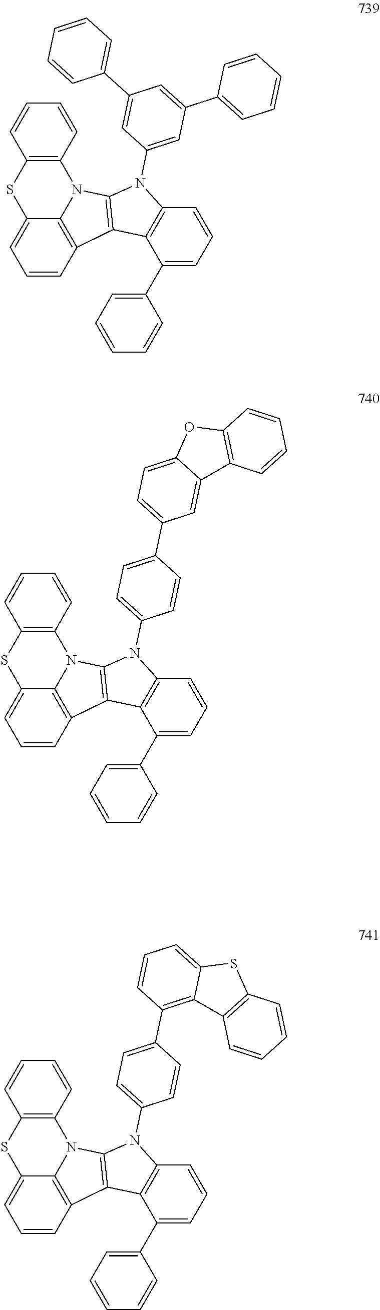 [SMILES: c1ccc(-c2cc(-c3ccccc3)cc(-n3c4cccc(-c5ccccc5)c4c4c5cccc6c5n(c43)-c3ccccc3S6)c2)cc1 ; c1ccc(-c2cccc3c2c2c4cccc5c4n(c2n3-c2ccc(-c3ccc4oc6ccccc6c4c3)cc2)-c2ccccc2S5)cc1 ; c1ccc(-c2cccc3c2c2c4cccc5c4n(c2n3-c2ccc(-c3cccc4sc6ccccc6c34)cc2)-c2ccccc2S5)cc1]

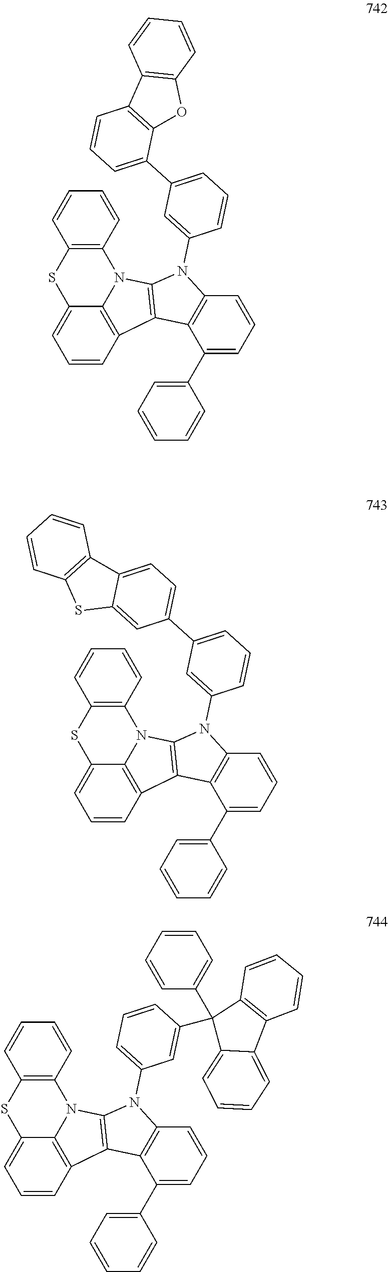

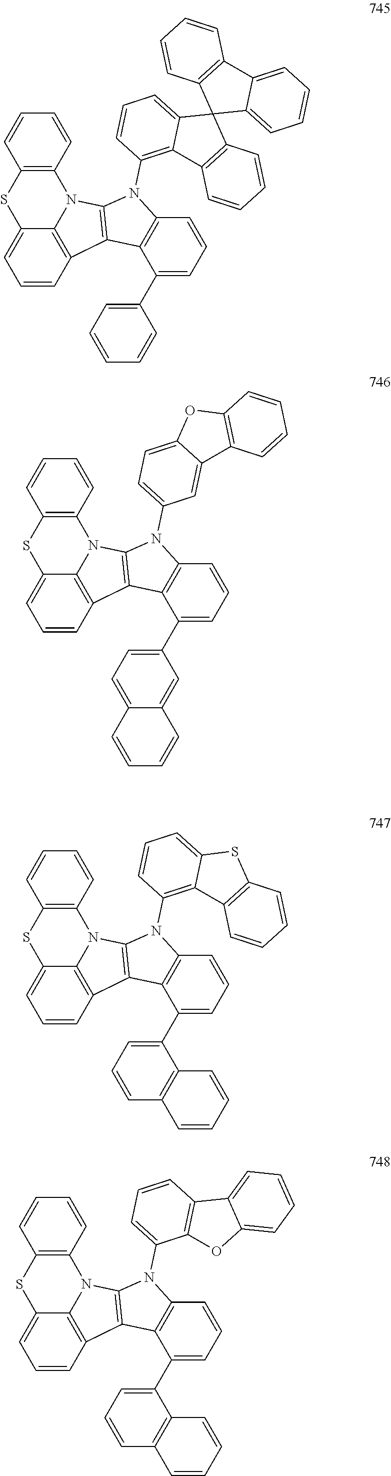 [SMILES: c1ccc(-c2cccc3c2c2c4cccc5c4n(c2n3-c2cccc3c2-c2ccccc2C32c3ccccc3-c3ccccc32)-c2ccccc2S5)cc1 ; c1ccc2c(c1)Sc1cccc3c4c5c(-c6ccc7ccccc7c6)cccc5n(-c5ccc6oc7ccccc7c6c5)c4n-2c13 ; c1ccc2c(c1)Sc1cccc3c4c5c(-c6cccc7ccccc67)cccc5n(-c5cccc6sc7ccccc7c56)c4n-2c13 ; c1ccc2c(c1)Sc1cccc3c4c5c(-c6cccc7ccccc67)cccc5n(-c5cccc6c5oc5ccccc56)c4n-2c13]

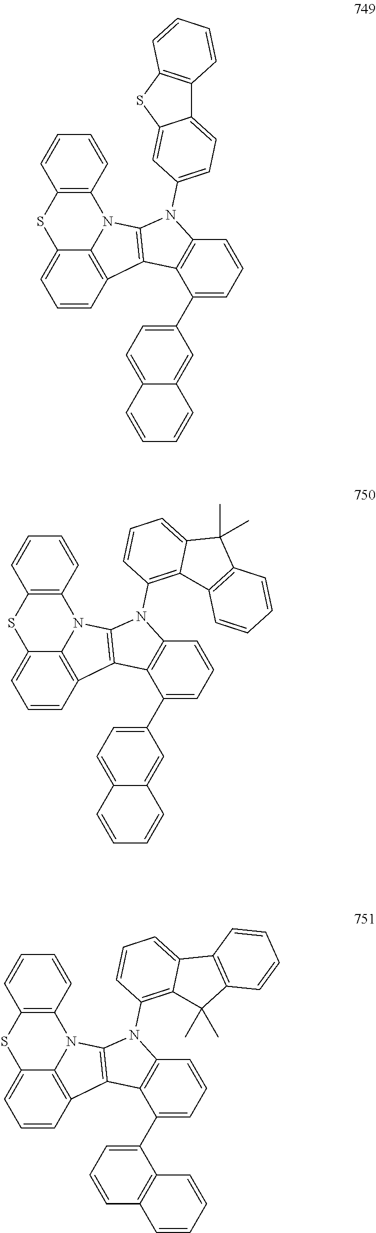 [SMILES: c1ccc2c(c1)Sc1cccc3c4c5c(-c6ccc7ccccc7c6)cccc5n(-c5ccc6c(c5)sc5ccccc56)c4n-2c13 ; CC1(C)c2ccccc2-c2c(-n3c4cccc(-c5ccc6ccccc6c5)c4c4c5cccc6c5n(c43)-c3ccccc3S6)cccc21 ; CC1(C)c2ccccc2-c2cccc(-n3c4cccc(-c5cccc6ccccc56)c4c4c5cccc6c5n(c43)-c3ccccc3S6)c21]

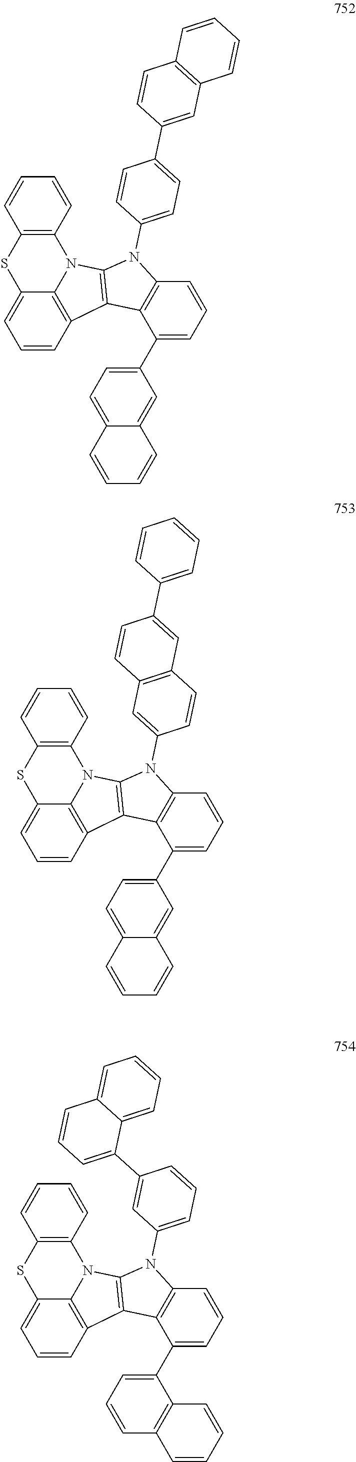

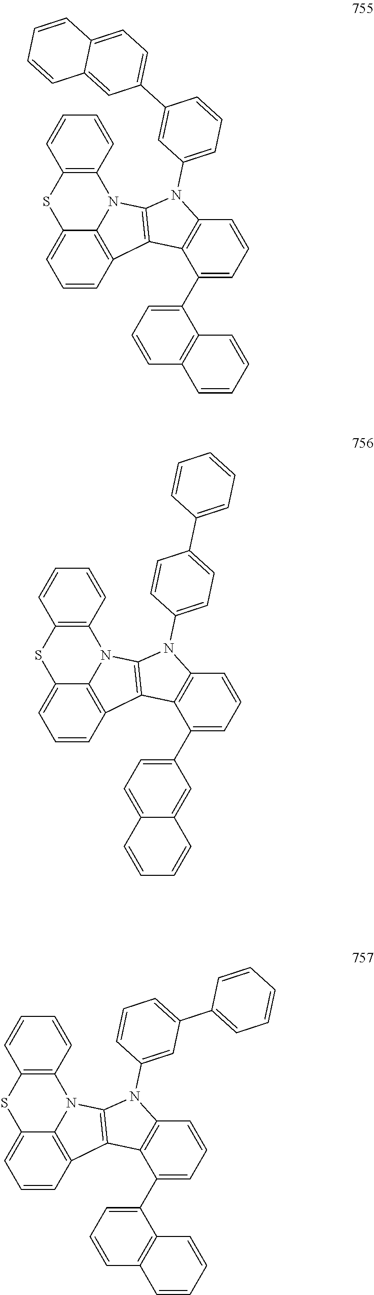 [SMILES: c1cc(-c2ccc3ccccc3c2)cc(-n2c3cccc(-c4cccc5ccccc45)c3c3c4cccc5c4n(c32)-c2ccccc2S5)c1 ; c1ccc(-c2ccc(-n3c4cccc(-c5ccc6ccccc6c5)c4c4c5cccc6c5n(c43)-c3ccccc3S6)cc2)cc1 ; c1ccc(-c2cccc(-n3c4cccc(-c5cccc6ccccc56)c4c4c5cccc6c5n(c43)-c3ccccc3S6)c2)cc1]

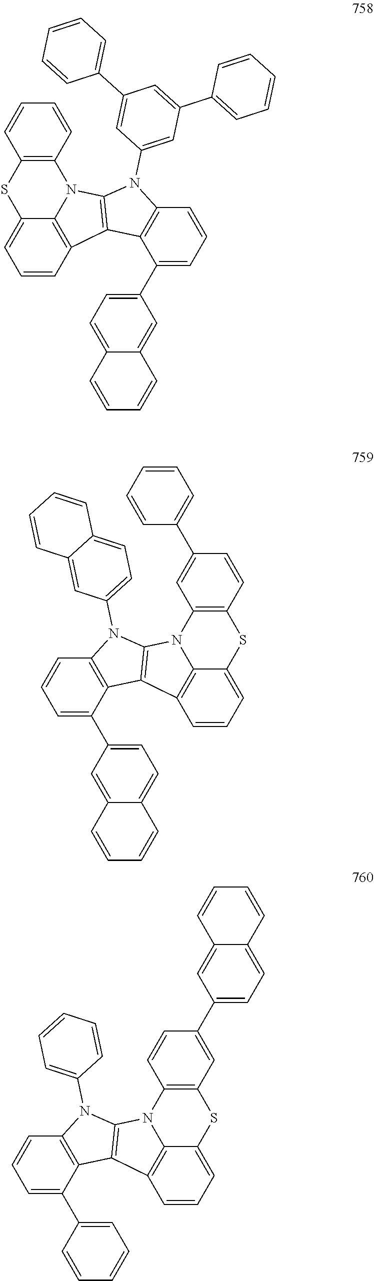 [SMILES: c1ccc(-c2cc(-c3ccccc3)cc(-n3c4cccc(-c5ccc6ccccc6c5)c4c4c5cccc6c5n(c43)-c3ccccc3S6)c2)cc1 ; c1ccc(-c2ccc3c(c2)-n2c4c(cccc4c4c5c(-c6ccc7ccccc7c6)cccc5n(-c5ccc6ccccc6c5)c42)S3)cc1 ; c1ccc(-c2cccc3c2c2c4cccc5c4n(c2n3-c2ccccc2)-c2ccc(-c3ccc4ccccc4c3)cc2S5)cc1]

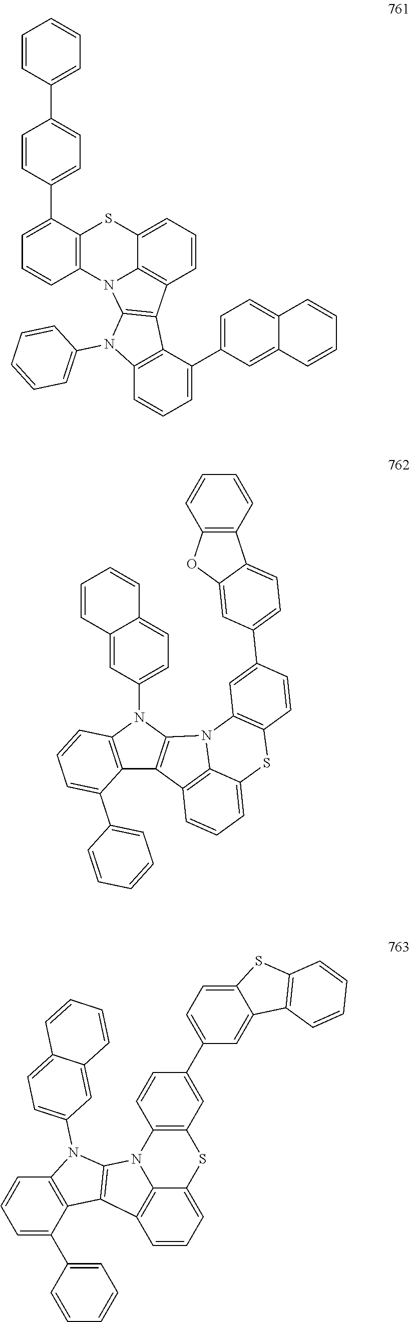 [SMILES: c1ccc(-c2ccc(-c3cccc4c3Sc3cccc5c6c7c(-c8ccc9ccccc9c8)cccc7n(-c7ccccc7)c6n-4c35)cc2)cc1 ; c1ccc(-c2cccc3c2c2c4cccc5c4n(c2n3-c2ccc3ccccc3c2)-c2cc(-c3ccc4c(c3)oc3ccccc34)ccc2S5)cc1 ; c1ccc(-c2cccc3c2c2c4cccc5c4n(c2n3-c2ccc3ccccc3c2)-c2ccc(-c3ccc4sc6ccccc6c4c3)cc2S5)cc1]

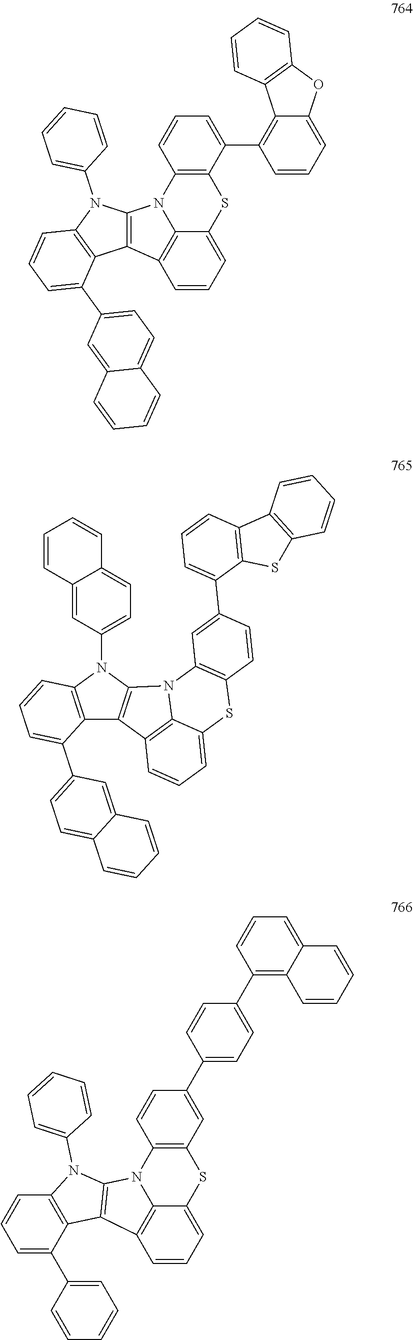 [SMILES: c1ccc(-n2c3cccc(-c4ccc5ccccc5c4)c3c3c4cccc5c4n(c32)-c2cccc(-c3cccc4oc6ccccc6c34)c2S5)cc1 ; c1ccc2cc(-c3cccc4c3c3c5cccc6c5n(c3n4-c3ccc4ccccc4c3)-c3cc(-c4cccc5c4sc4ccccc45)ccc3S6)ccc2c1 ; c1ccc(-c2cccc3c2c2c4cccc5c4n(c2n3-c2ccccc2)-c2ccc(-c3ccc(-c4cccc6ccccc46)cc3)cc2S5)cc1]

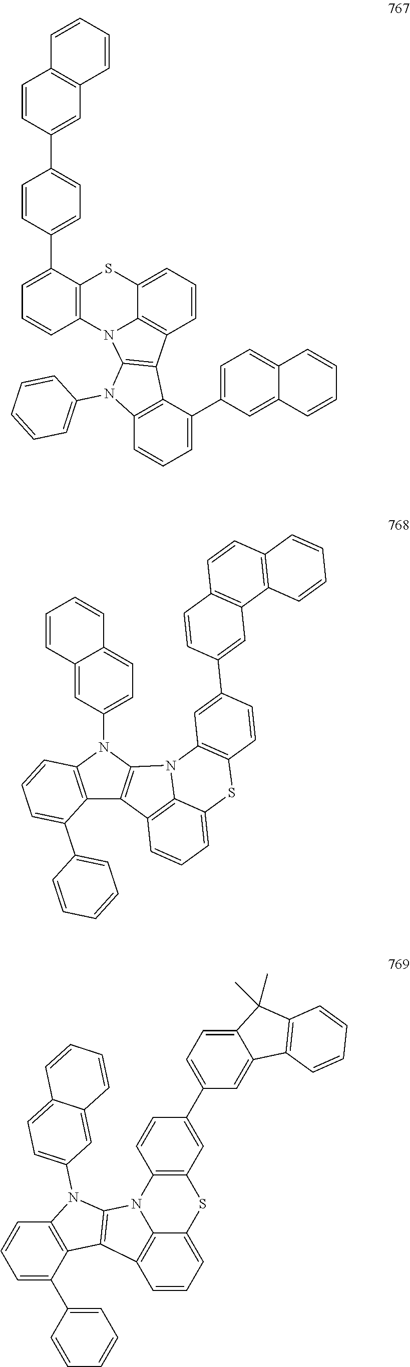 [SMILES: c1ccc(-n2c3cccc(-c4ccc5ccccc5c4)c3c3c4cccc5c4n(c32)-c2cccc(-c3ccc(-c4ccc6ccccc6c4)cc3)c2S5)cc1 ; c1ccc(-c2cccc3c2c2c4cccc5c4n(c2n3-c2ccc3ccccc3c2)-c2cc(-c3ccc4ccc6ccccc6c4c3)ccc2S5)cc1 ; CC1(C)c2ccccc2-c2cc(-c3ccc4c(c3)Sc3cccc5c6c7c(-c8ccccc8)cccc7n(-c7ccc8ccccc8c7)c6n-4c35)ccc21]

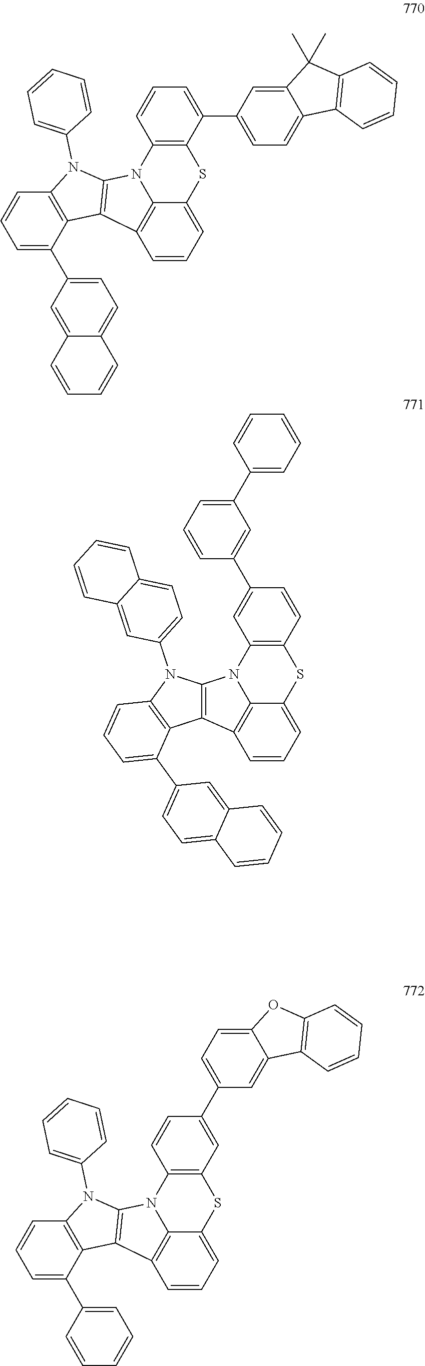 [SMILES: CC1(C)c2ccccc2-c2ccc(-c3cccc4c3Sc3cccc5c6c7c(-c8ccc9ccccc9c8)cccc7n(-c7ccccc7)c6n-4c35)cc21 ; c1ccc(-c2cccc(-c3ccc4c(c3)-n3c5c(cccc5c5c6c(-c7ccc8ccccc8c7)cccc6n(-c6ccc7ccccc7c6)c53)S4)c2)cc1 ; c1ccc(-c2cccc3c2c2c4cccc5c4n(c2n3-c2ccccc2)-c2ccc(-c3ccc4oc6ccccc6c4c3)cc2S5)cc1]

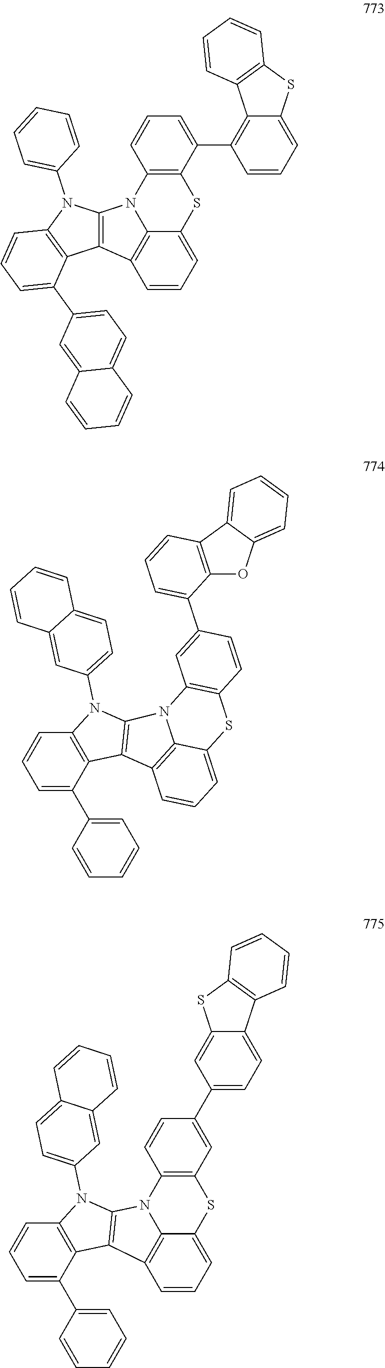 [SMILES: c1ccc(-n2c3cccc(-c4ccc5ccccc5c4)c3c3c4cccc5c4n(c32)-c2cccc(-c3cccc4sc6ccccc6c34)c2S5)cc1 ; c1ccc(-c2cccc3c2c2c4cccc5c4n(c2n3-c2ccc3ccccc3c2)-c2cc(-c3cccc4c3oc3ccccc34)ccc2S5)cc1 ; c1ccc(-c2cccc3c2c2c4cccc5c4n(c2n3-c2ccc3ccccc3c2)-c2ccc(-c3ccc4c(c3)sc3ccccc34)cc2S5)cc1]

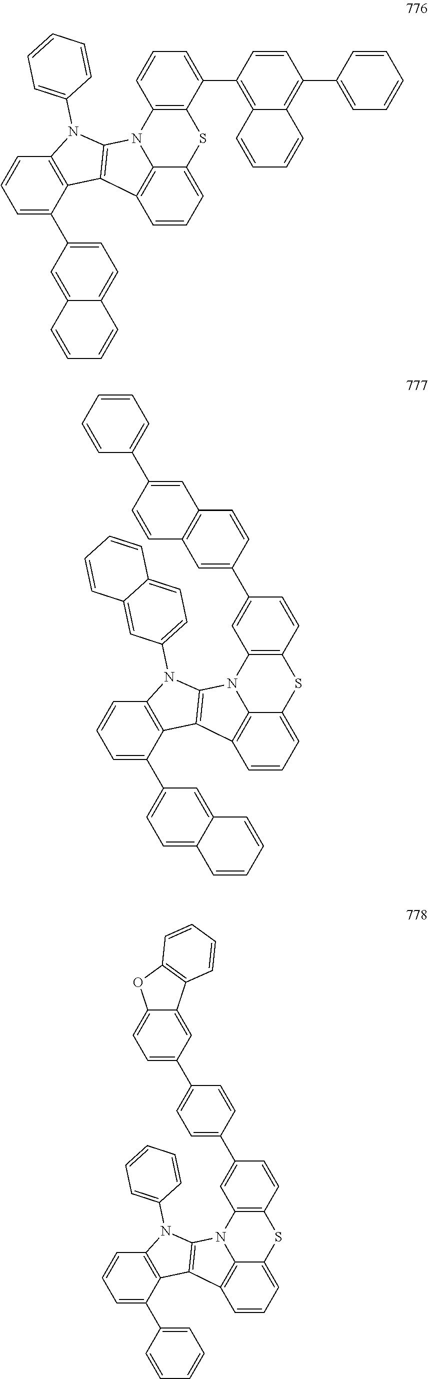

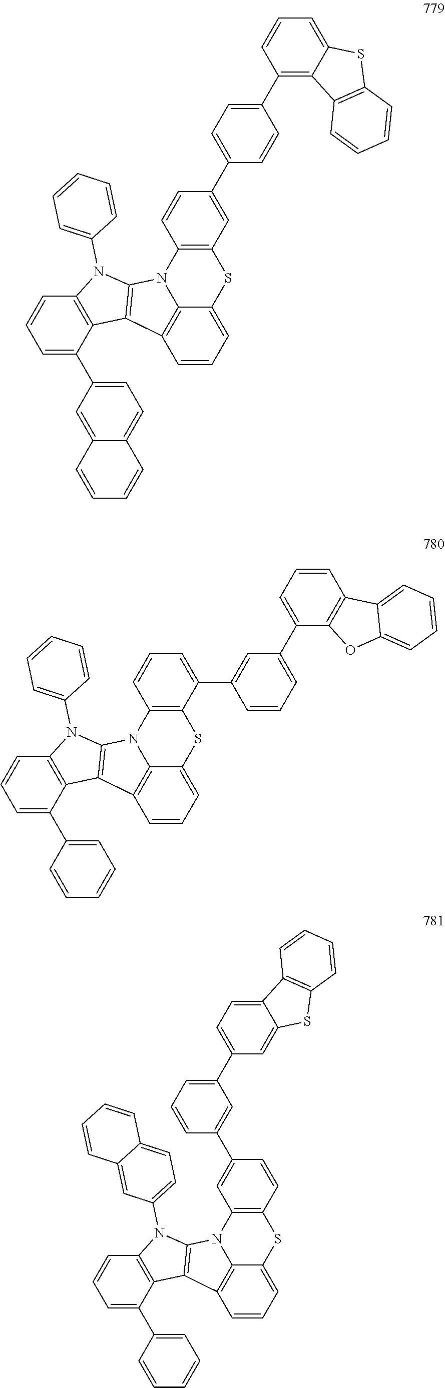 [SMILES: c1ccc(-n2c3cccc(-c4ccc5ccccc5c4)c3c3c4cccc5c4n(c32)-c2ccc(-c3ccc(-c4cccc6sc7ccccc7c46)cc3)cc2S5)cc1 ; c1ccc(-c2cccc3c2c2c4cccc5c4n(c2n3-c2ccccc2)-c2cccc(-c3cccc(-c4cccc6c4oc4ccccc46)c3)c2S5)cc1 ; c1ccc(-c2cccc3c2c2c4cccc5c4n(c2n3-c2ccc3ccccc3c2)-c2cc(-c3cccc(-c4ccc6c(c4)sc4ccccc46)c3)ccc2S5)cc1]

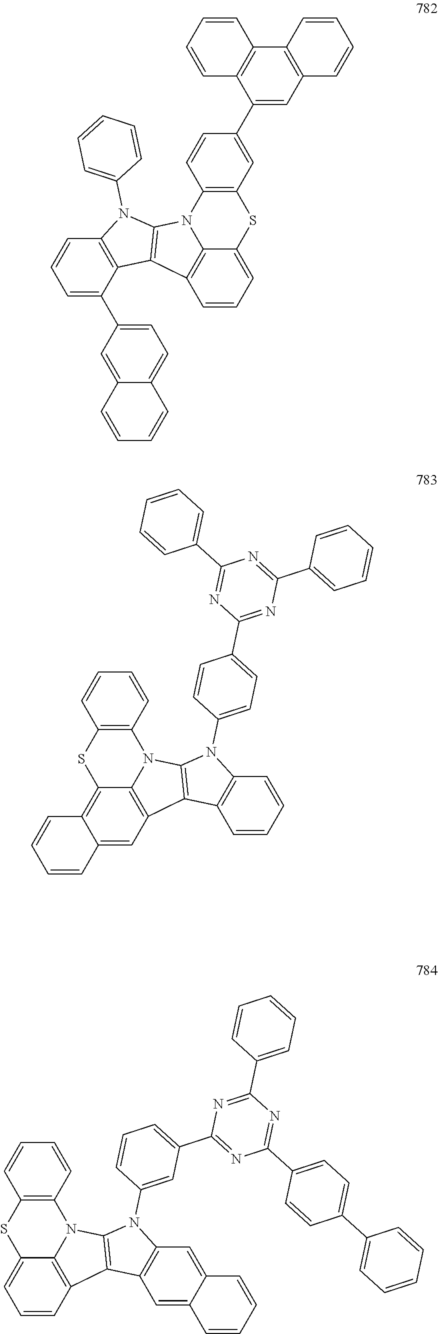 [SMILES: c1ccc(-n2c3cccc(-c4ccc5ccccc5c4)c3c3c4cccc5c4n(c32)-c2ccc(-c3cc4ccccc4c4ccccc34)cc2S5)cc1 ; c1ccc(-c2nc(-c3ccccc3)nc(-c3ccc(-n4c5ccccc5c5c6cc7ccccc7c7c6n(c54)-c4ccccc4S7)cc3)n2)cc1 ; c1ccc(-c2ccc(-c3nc(-c4ccccc4)nc(-c4cccc(-n5c6cc7ccccc7cc6c6c7cccc8c7n(c65)-c5ccccc5S8)c4)n3)cc2)cc1]

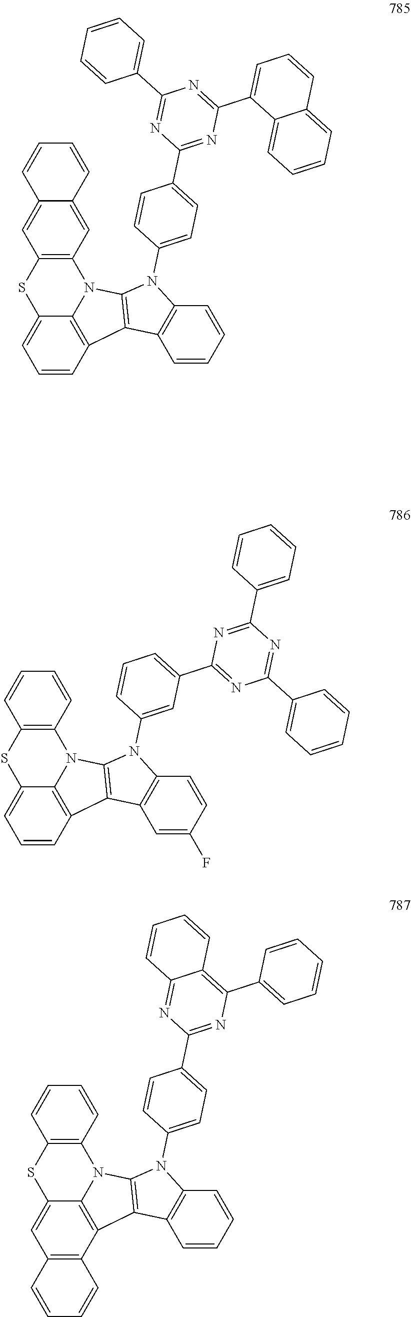 [SMILES: c1ccc(-c2nc(-c3ccc(-n4c5ccccc5c5c6cccc7c6n(c54)-c4cc5ccccc5cc4S7)cc3)nc(-c3cccc4ccccc34)n2)cc1 ; Fc1ccc2c(c1)c1c3cccc4c3n(c1n2-c1cccc(-c2nc(-c3ccccc3)nc(-c3ccccc3)n2)c1)-c1ccccc1S4 ; c1ccc(-c2nc(-c3ccc(-n4c5ccccc5c5c6c7ccccc7cc7c6n(c54)-c4ccccc4S7)cc3)nc3ccccc23)cc1]

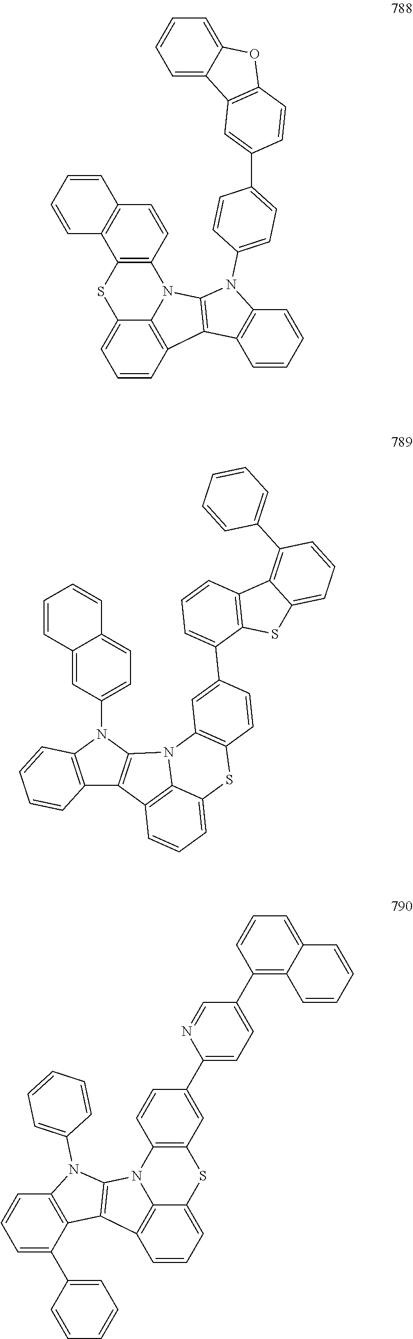 [SMILES: c1ccc2c3c(ccc2c1)-n1c2c(cccc2c2c4ccccc4n(-c4ccc(-c5ccc6oc7ccccc7c6c5)cc4)c21)S3 ; c1ccc(-c2cccc3sc4c(-c5ccc6c(c5)-n5c7c(cccc7c7c8ccccc8n(-c8ccc9ccccc9c8)c75)S6)cccc4c23)cc1 ; c1ccc(-c2cccc3c2c2c4cccc5c4n(c2n3-c2ccccc2)-c2ccc(-c3ccc(-c4cccc6ccccc46)cn3)cc2S5)cc1]

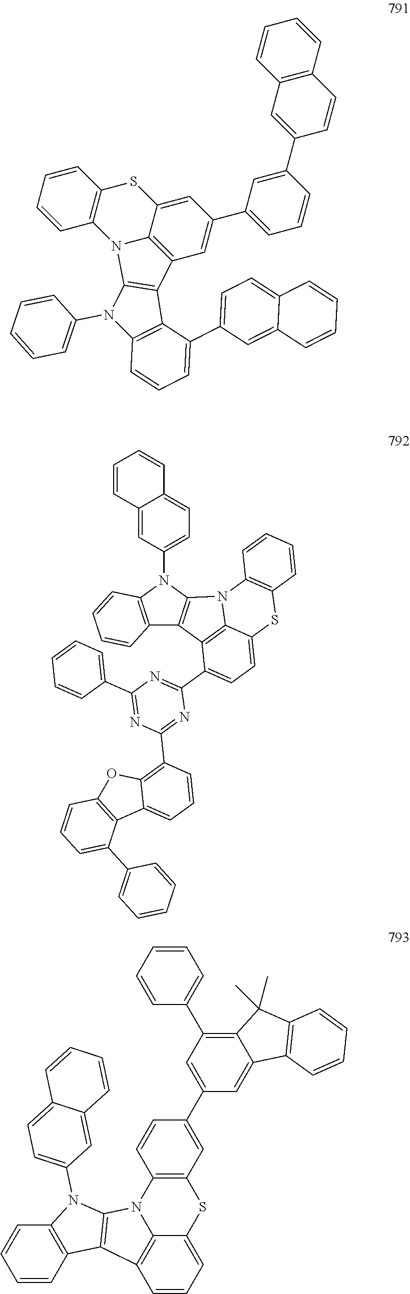

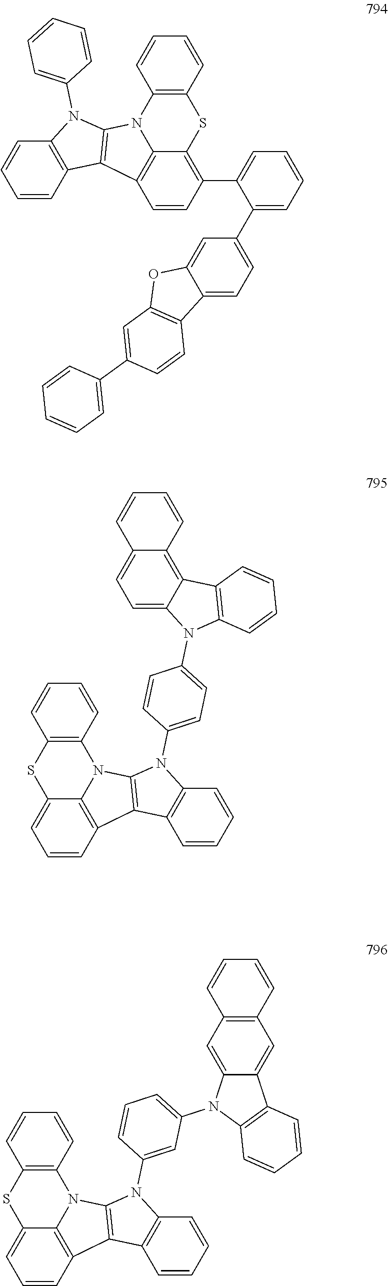 [SMILES: c1ccc(-c2ccc3c(c2)oc2cc(-c4ccccc4-c4ccc5c6c7ccccc7n(-c7ccccc7)c6n6c5c4Sc4ccccc4-6)ccc23)cc1 ; c1ccc2c(c1)Sc1cccc3c4c5ccccc5n(-c5ccc(-n6c7ccccc7c7c8ccccc8ccc76)cc5)c4n-2c13 ; c1cc(-n2c3ccccc3c3cc4ccccc4cc32)cc(-n2c3ccccc3c3c4cccc5c4n(c32)-c2ccccc2S5)c1]

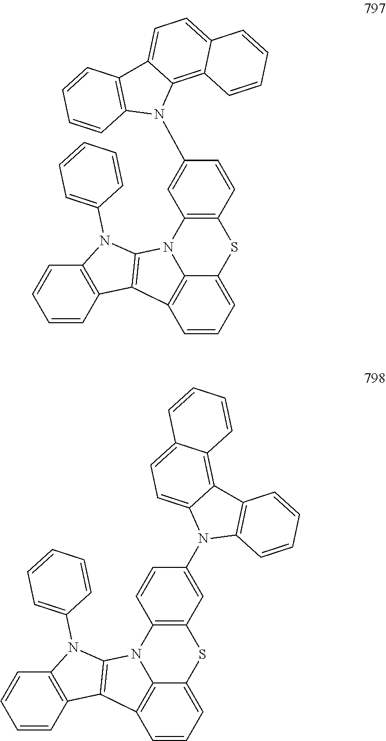 [SMILES: c1ccc(-n2c3ccccc3c3c4cccc5c4n(c32)-c2cc(-n3c4ccccc4c4ccc6ccccc6c43)ccc2S5)cc1 ; c1ccc(-n2c3ccccc3c3c4cccc5c4n(c32)-c2ccc(-n3c4ccccc4c4c6ccccc6ccc43)cc2S5)cc1]

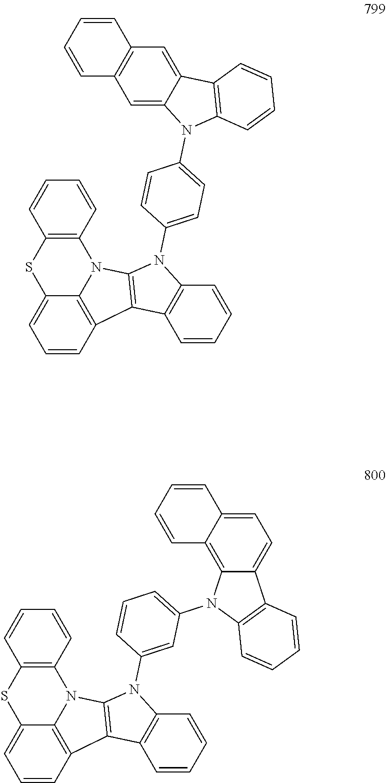 [SMILES: c1ccc2c(c1)Sc1cccc3c4c5ccccc5n(-c5ccc(-n6c7ccccc7c7cc8ccccc8cc76)cc5)c4n-2c13 ; c1cc(-n2c3ccccc3c3ccc4ccccc4c32)cc(-n2c3ccccc3c3c4cccc5c4n(c32)-c2ccccc2S5)c1]

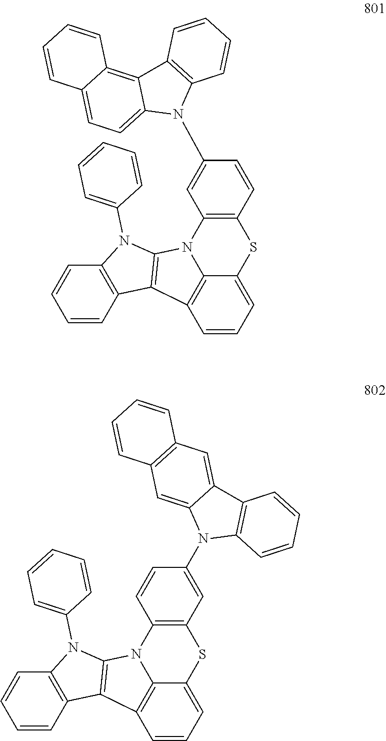 [SMILES: c1ccc(-n2c3ccccc3c3c4cccc5c4n(c32)-c2cc(-n3c4ccccc4c4c6ccccc6ccc43)ccc2S5)cc1 ; c1ccc(-n2c3ccccc3c3c4cccc5c4n(c32)-c2ccc(-n3c4ccccc4c4cc6ccccc6cc43)cc2S5)cc1]

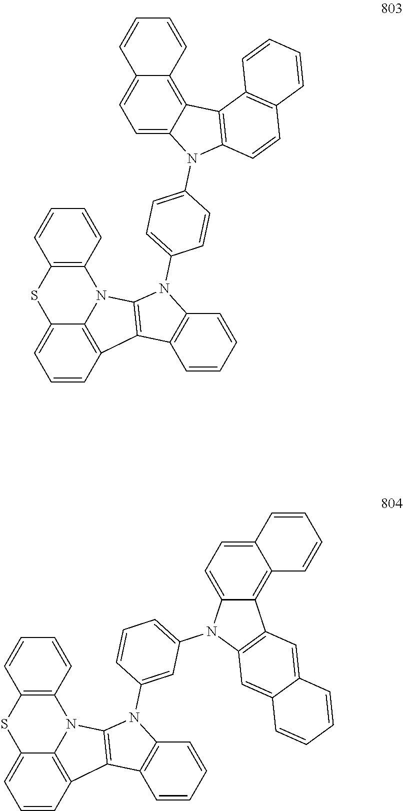 [SMILES: c1ccc2c(c1)Sc1cccc3c4c5ccccc5n(-c5ccc(-n6c7ccc8ccccc8c7c7c8ccccc8ccc76)cc5)c4n-2c13 ; c1cc(-n2c3cc4ccccc4cc3c3c4ccccc4ccc32)cc(-n2c3ccccc3c3c4cccc5c4n(c32)-c2ccccc2S5)c1]

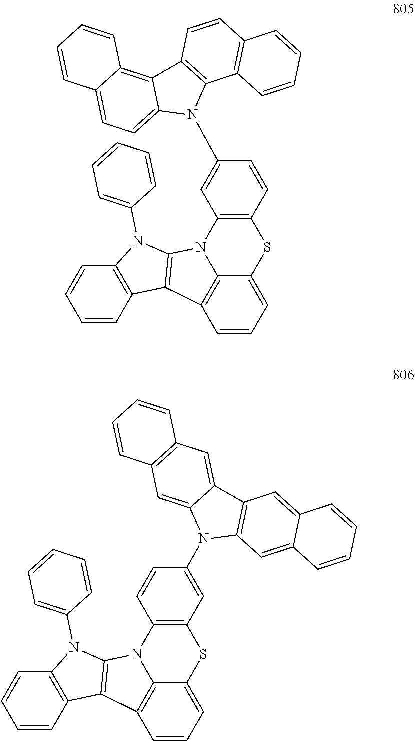 [SMILES: c1ccc(-n2c3ccccc3c3c4cccc5c4n(c32)-c2cc(-n3c4ccc6ccccc6c4c4ccc6ccccc6c43)ccc2S5)cc1 ; c1ccc(-n2c3ccccc3c3c4cccc5c4n(c32)-c2ccc(-n3c4cc6ccccc6cc4c4cc6ccccc6cc43)cc2S5)cc1]

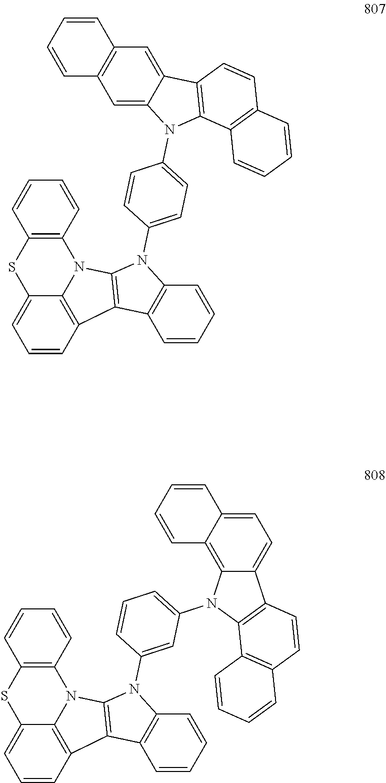 [SMILES: c1ccc2c(c1)Sc1cccc3c4c5ccccc5n(-c5ccc(-n6c7cc8ccccc8cc7c7ccc8ccccc8c76)cc5)c4n-2c13 ; c1cc(-n2c3c4ccccc4ccc3c3ccc4ccccc4c32)cc(-n2c3ccccc3c3c4cccc5c4n(c32)-c2ccccc2S5)c1]

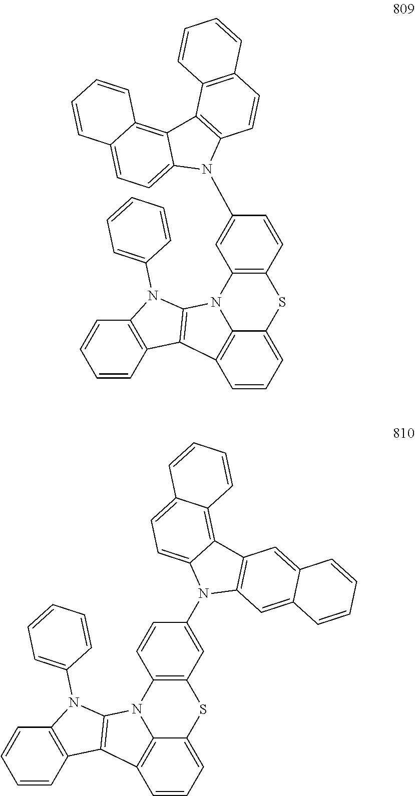 [SMILES: c1ccc(-n2c3ccccc3c3c4cccc5c4n(c32)-c2cc(-n3c4ccc6ccccc6c4c4c6ccccc6ccc43)ccc2S5)cc1 ; c1ccc(-n2c3ccccc3c3c4cccc5c4n(c32)-c2ccc(-n3c4cc6ccccc6cc4c4c6ccccc6ccc43)cc2S5)cc1]

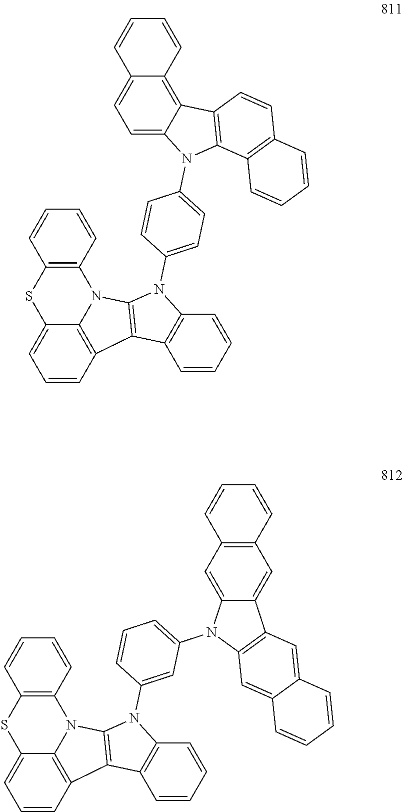 [SMILES: c1ccc2c(c1)Sc1cccc3c4c5ccccc5n(-c5ccc(-n6c7ccc8ccccc8c7c7ccc8ccccc8c76)cc5)c4n-2c13 ; c1cc(-n2c3cc4ccccc4cc3c3cc4ccccc4cc32)cc(-n2c3ccccc3c3c4cccc5c4n(c32)-c2ccccc2S5)c1]

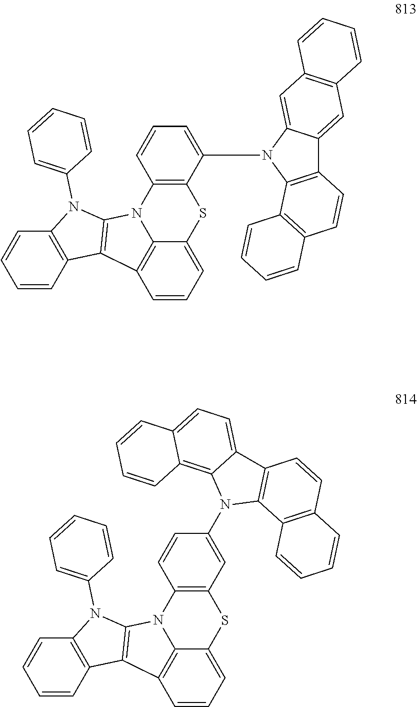 [SMILES: c1ccc(-n2c3ccccc3c3c4cccc5c4n(c32)-c2cccc(-n3c4cc6ccccc6cc4c4ccc6ccccc6c43)c2S5)cc1 ; c1ccc(-n2c3ccccc3c3c4cccc5c4n(c32)-c2ccc(-n3c4c6ccccc6ccc4c4ccc6ccccc6c43)cc2S5)cc1]

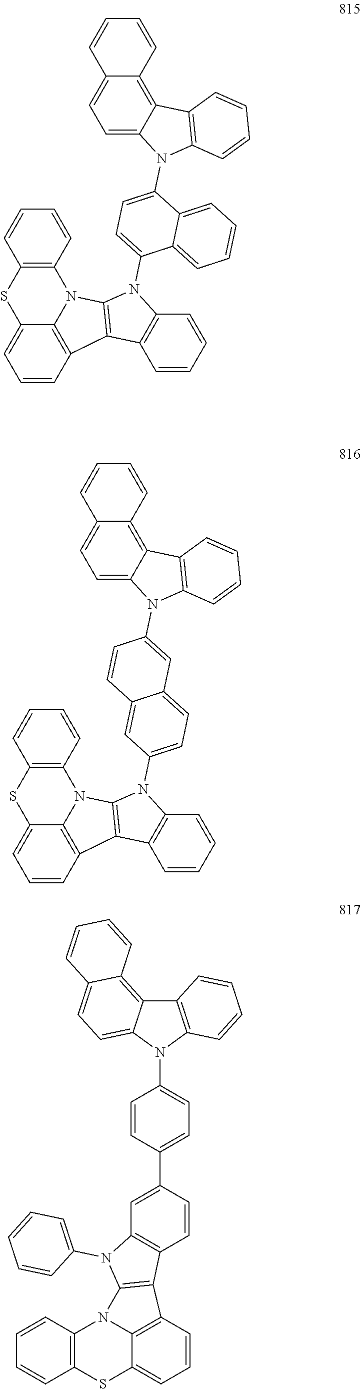

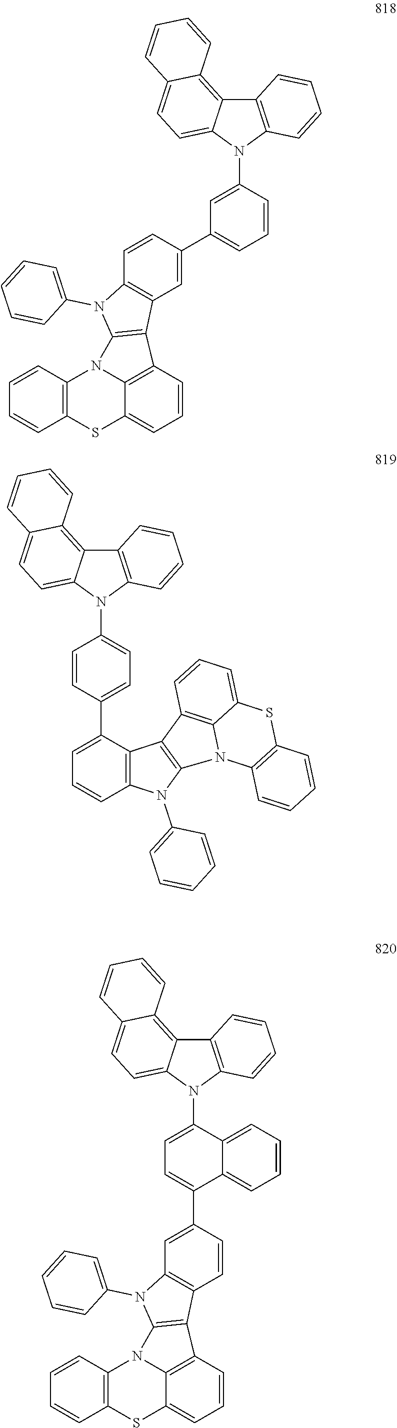

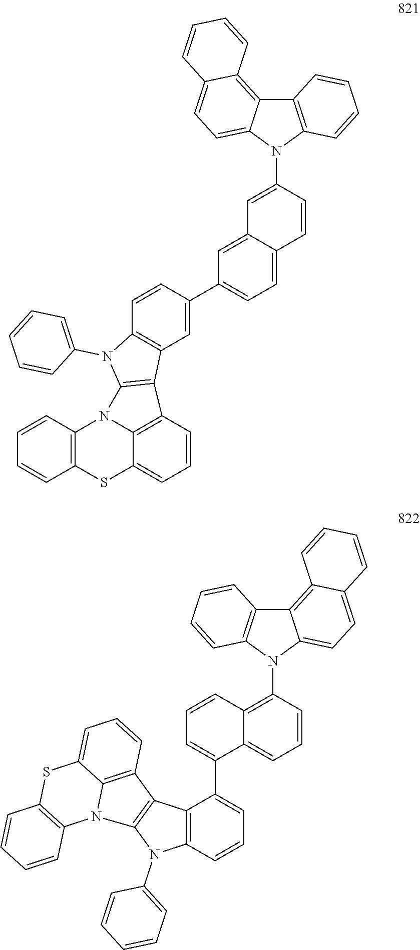 [SMILES: c1ccc(-n2c3ccc(-c4ccc5ccc(-n6c7ccccc7c7c8ccccc8ccc76)cc5c4)cc3c3c4cccc5c4n(c32)-c2ccccc2S5)cc1 ; c1ccc(-n2c3cccc(-c4cccc5c(-n6c7ccccc7c7c8ccccc8ccc76)cccc45)c3c3c4cccc5c4n(c32)-c2ccccc2S5)cc1]

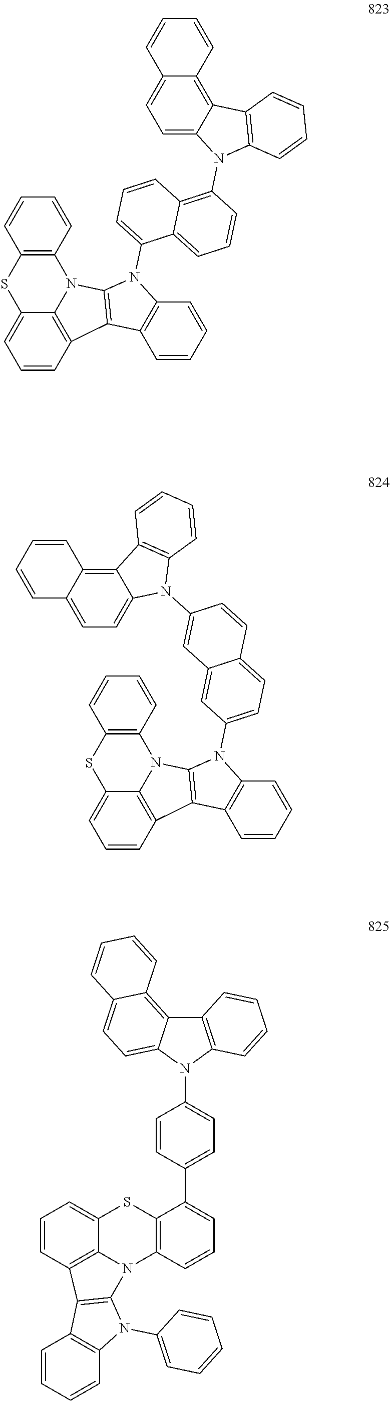 [SMILES: c1ccc2c(c1)Sc1cccc3c4c5ccccc5n(-c5cccc6c(-n7c8ccccc8c8c9ccccc9ccc87)cccc56)c4n-2c13 ; c1ccc2c(c1)Sc1cccc3c4c5ccccc5n(-c5ccc6ccc(-n7c8ccccc8c8c9ccccc9ccc87)cc6c5)c4n-2c13 ; c1ccc(-n2c3ccccc3c3c4cccc5c4n(c32)-c2cccc(-c3ccc(-n4c6ccccc6c6c7ccccc7ccc64)cc3)c2S5)cc1]

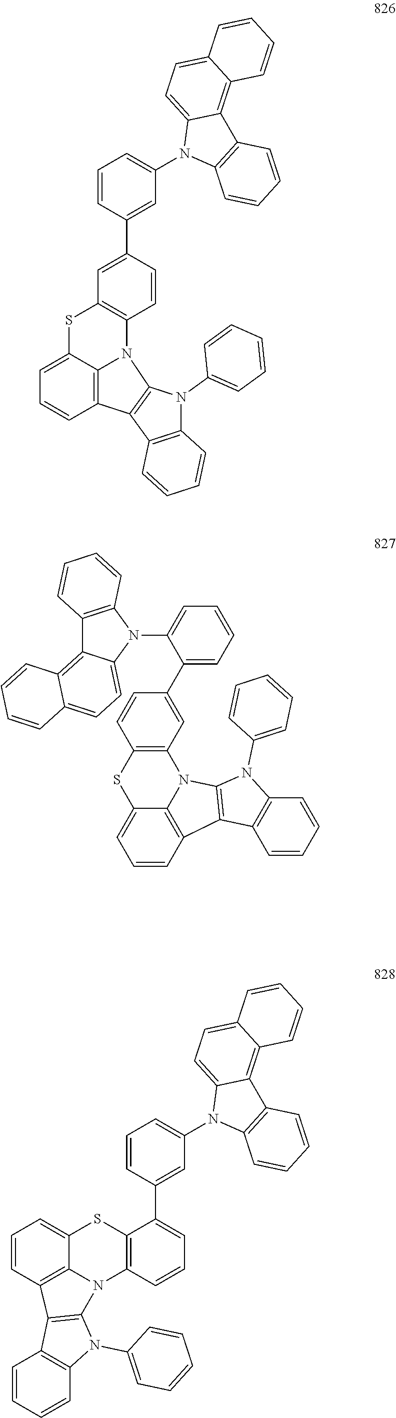 [SMILES: c1ccc(-n2c3ccccc3c3c4cccc5c4n(c32)-c2ccc(-c3cccc(-n4c6ccccc6c6c7ccccc7ccc64)c3)cc2S5)cc1 ; c1ccc(-n2c3ccccc3c3c4cccc5c4n(c32)-c2cc(-c3ccccc3-n3c4ccccc4c4c6ccccc6ccc43)ccc2S5)cc1 ; c1ccc(-n2c3ccccc3c3c4cccc5c4n(c32)-c2cccc(-c3cccc(-n4c6ccccc6c6c7ccccc7ccc64)c3)c2S5)cc1]

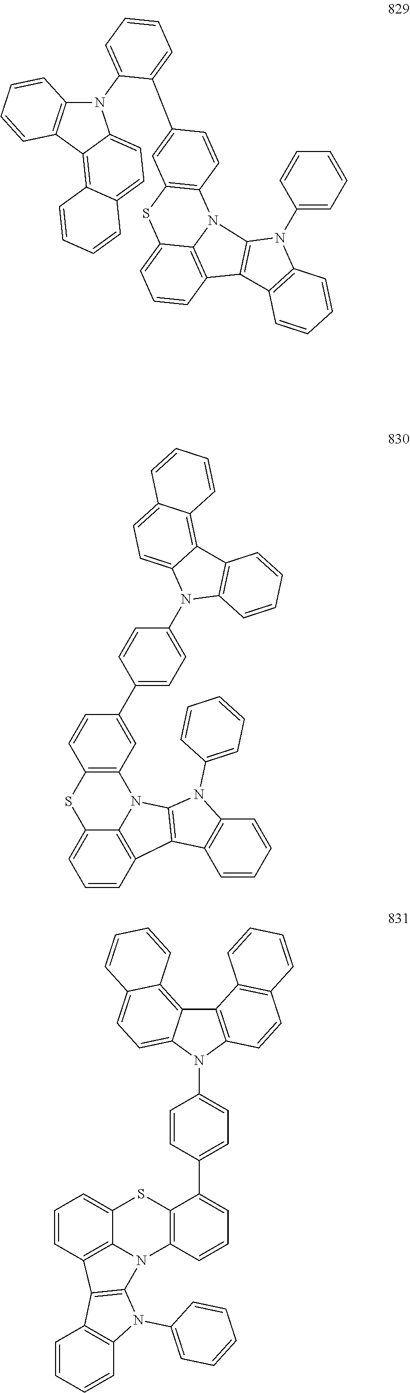 [SMILES: c1ccc(-n2c3ccccc3c3c4cccc5c4n(c32)-c2ccc(-c3ccccc3-n3c4ccccc4c4c6ccccc6ccc43)cc2S5)cc1 ; c1ccc(-n2c3ccccc3c3c4cccc5c4n(c32)-c2cc(-c3ccc(-n4c6ccccc6c6c7ccccc7ccc64)cc3)ccc2S5)cc1 ; c1ccc(-n2c3ccccc3c3c4cccc5c4n(c32)-c2cccc(-c3ccc(-n4c6ccc7ccccc7c6c6c7ccccc7ccc64)cc3)c2S5)cc1]

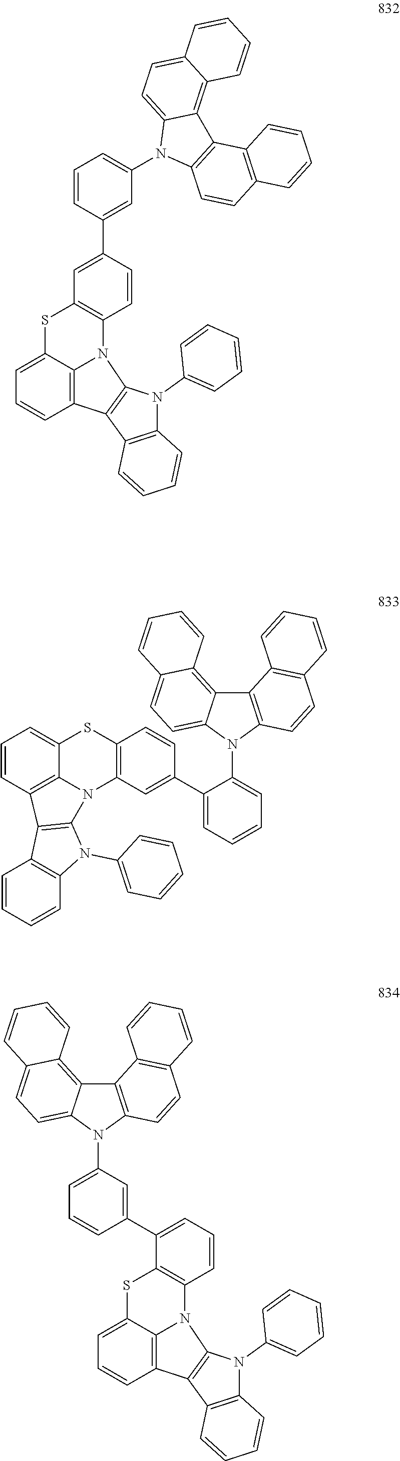 [SMILES: c1ccc(-n2c3ccccc3c3c4cccc5c4n(c32)-c2ccc(-c3cccc(-n4c6ccc7ccccc7c6c6c7ccccc7ccc64)c3)cc2S5)cc1 ; c1ccc(-n2c3ccccc3c3c4cccc5c4n(c32)-c2cc(-c3ccccc3-n3c4ccc6ccccc6c4c4c6ccccc6ccc43)ccc2S5)cc1 ; c1ccc(-n2c3ccccc3c3c4cccc5c4n(c32)-c2cccc(-c3cccc(-n4c6ccc7ccccc7c6c6c7ccccc7ccc64)c3)c2S5)cc1]

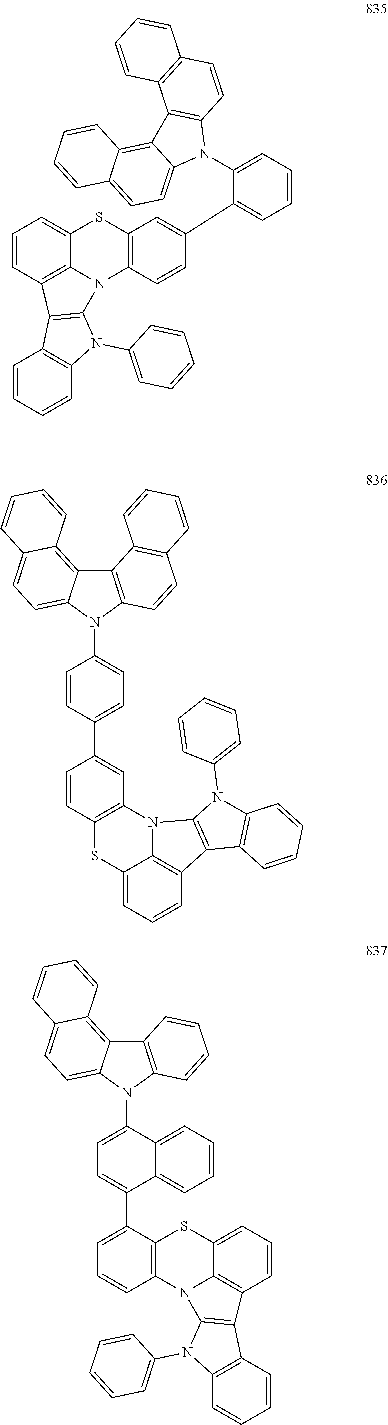

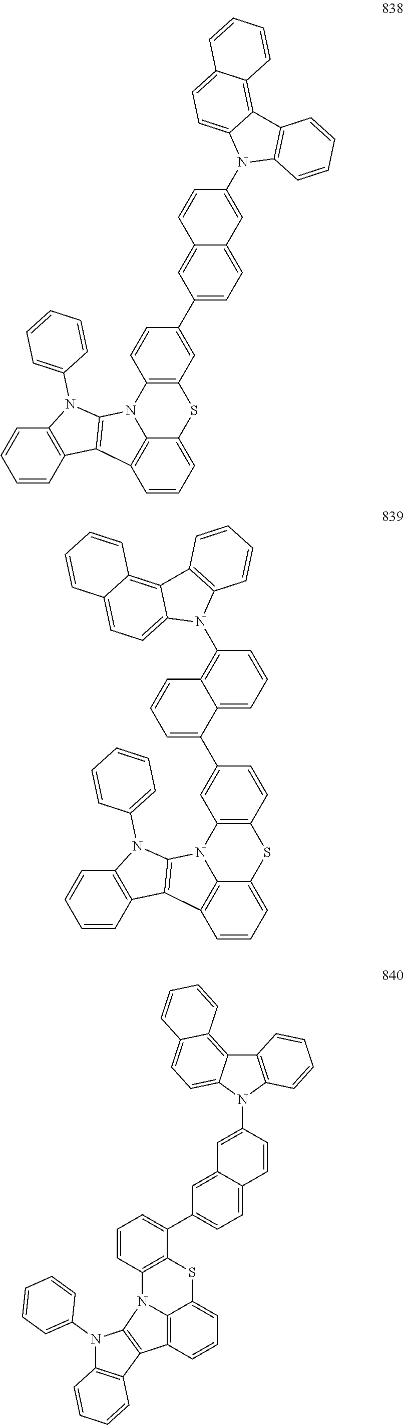 [SMILES: c1ccc(-n2c3ccccc3c3c4cccc5c4n(c32)-c2ccc(-c3ccc4cc(-n6c7ccccc7c7c8ccccc8ccc76)ccc4c3)cc2S5)cc1 ; c1ccc(-n2c3ccccc3c3c4cccc5c4n(c32)-c2cc(-c3cccc4c(-n6c7ccccc7c7c8ccccc8ccc76)cccc34)ccc2S5)cc1 ; c1ccc(-n2c3ccccc3c3c4cccc5c4n(c32)-c2cccc(-c3ccc4ccc(-n6c7ccccc7c7c8ccccc8ccc76)cc4c3)c2S5)cc1]

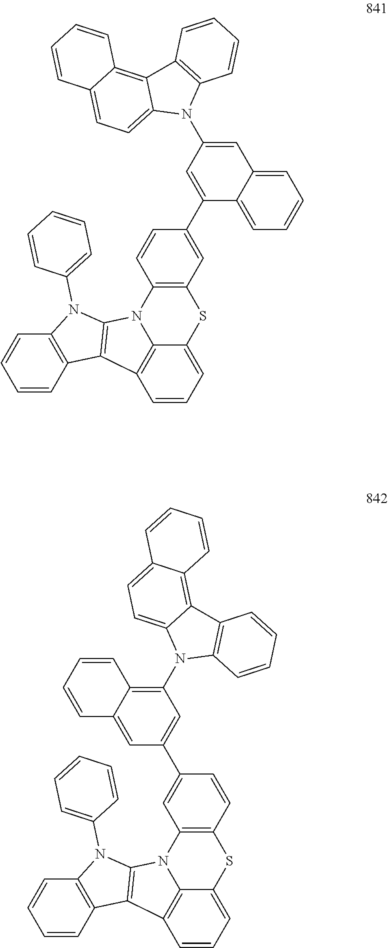 [SMILES: c1ccc(-n2c3ccccc3c3c4cccc5c4n(c32)-c2ccc(-c3cc(-n4c6ccccc6c6c7ccccc7ccc64)cc4ccccc34)cc2S5)cc1 ; c1ccc(-n2c3ccccc3c3c4cccc5c4n(c32)-c2cc(-c3cc(-n4c6ccccc6c6c7ccccc7ccc64)c4ccccc4c3)ccc2S5)cc1]

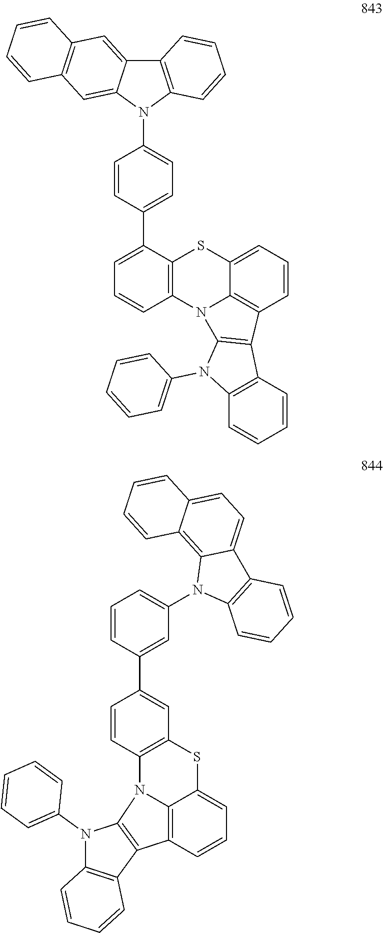 [SMILES: c1ccc(-n2c3ccccc3c3c4cccc5c4n(c32)-c2cccc(-c3ccc(-n4c6ccccc6c6cc7ccccc7cc64)cc3)c2S5)cc1 ; c1ccc(-n2c3ccccc3c3c4cccc5c4n(c32)-c2ccc(-c3cccc(-n4c6ccccc6c6ccc7ccccc7c64)c3)cc2S5)cc1]

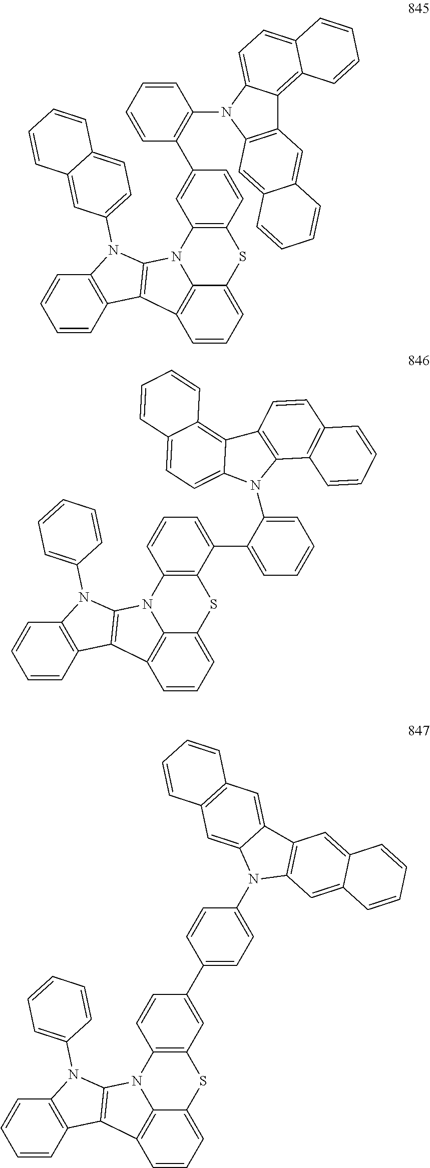

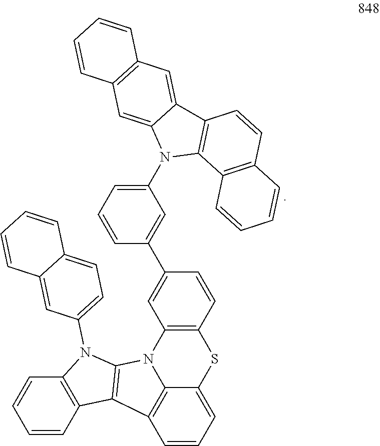 [SMILES: c1cc(-c2ccc3c(c2)-n2c4c(cccc4c4c5ccccc5n(-c5ccc6ccccc6c5)c42)S3)cc(-n2c3cc4ccccc4cc3c3ccc4ccccc4c32)c1]

The present disclosure, in a second aspect, provides an organic electroluminescent device. The organic electroluminescent device comprises an anode, a cathode, and a functional layer disposed between the anode and the cathode. The functional layer comprises the nitrogen-containing compound described in the first aspect of the present disclosure.

The nitrogen-containing compound provided in the present disclosure may be used to form at least one organic film layer in the functional layer so as to improve properties of the organic electroluminescent device such as luminescence efficiency and service life.

Optionally, the functional layer comprises an organic luminescent layer comprising the nitrogen-containing compound. The organic luminescent layer may be composed of the nitrogen-containing compound provided in the present disclosure, or may be composed of the nitrogen-containing compound provided in the present disclosure together with other materials.

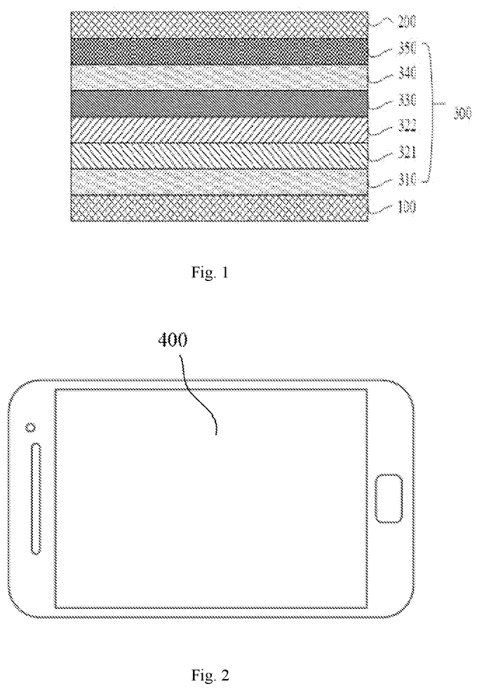

According to a specific embodiment, the organic electroluminescent device is as shown in FIG. 1. The organic electroluminescent device may comprise an anode 100, a hole injection layer 310, a first hole transport layer 321, a second hole transport layer (hole auxiliary layer) 322, an organic luminescent layer 330, an electron transport layer 340, an electron injection layer 350, and a cathode 200 that are stacked in sequence.

In the present disclosure, the anode 100 comprises an anode material, which is preferably a high-work function material contributing to injection of holes into the functional layer. Specific examples of the anode material include, but are not limited to: metals such as nickel, platinum, vanadium, chromium, copper, zinc, gold, and alloys thereof; metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy)thiophene] (PEDT), polypyrrole, and polyaniline. Preferably, a transparent electrode comprising indium tin oxide (ITO) is included as the anode.

In the present disclosure, the hole transport layer may comprise one or more hole transport materials. The transport materials may be selected from carbazole polymers, carbazole-linked triarylamine compounds, and other types of compounds, and may be specifically selected from the following compounds or any combinations thereof:

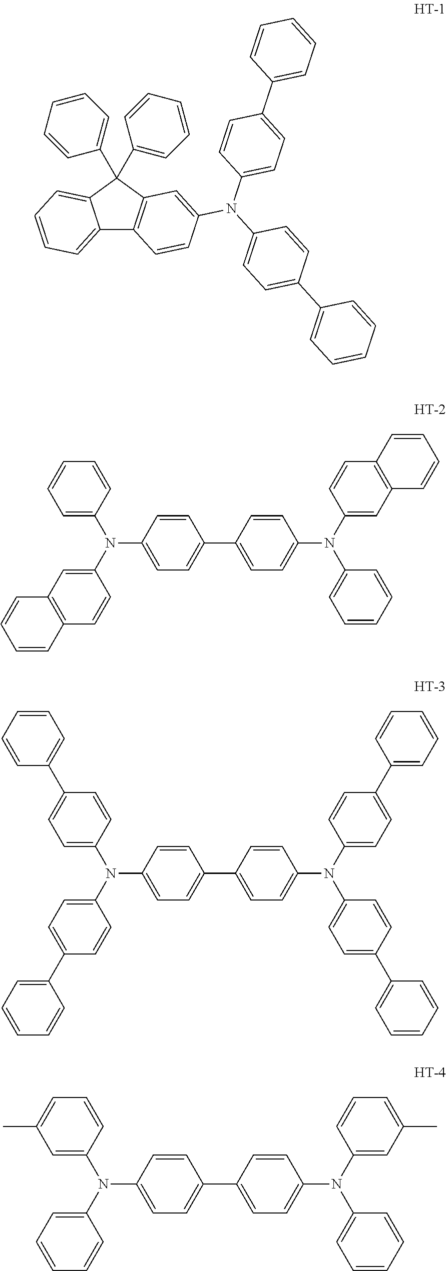

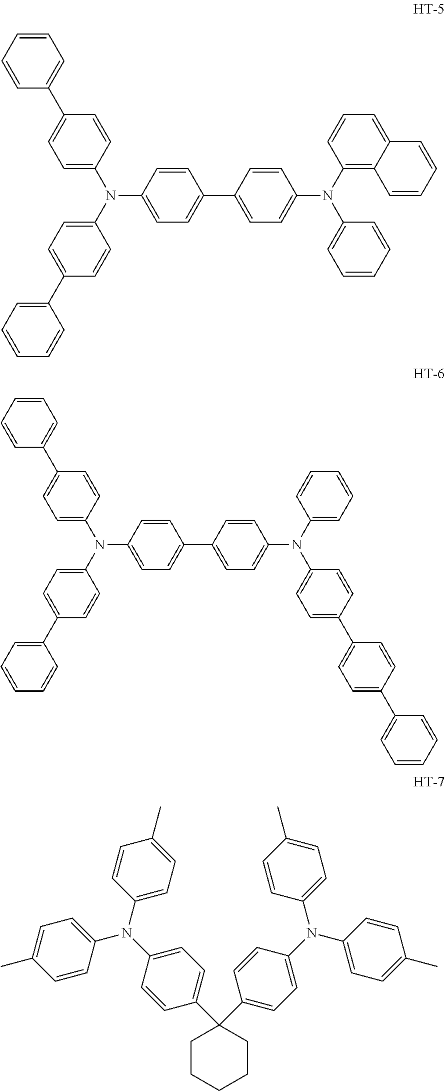

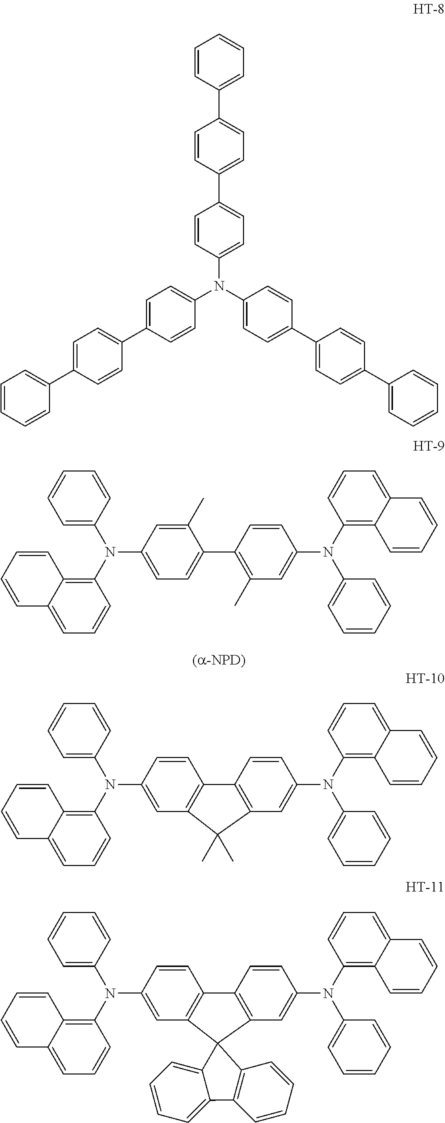 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; Cc1cc(N(c2ccccc2)c2cccc3ccccc23)ccc1-c1ccc(N(c2ccccc2)c2cccc3ccccc23)cc1C ; CC1(C)c2cc(N(c3ccccc3)c3cccc4ccccc34)ccc2-c2ccc(N(c3ccccc3)c3cccc4ccccc34)cc21 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

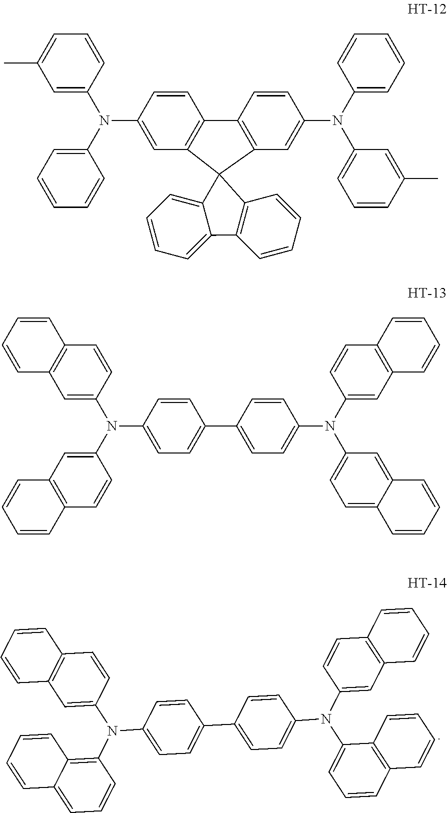

In an embodiment, the first hole transport layer 321 is composed of α-NPD.

In an embodiment, the second hole transport layer 322 is composed of HT-1.

Optionally, a hole injection layer 310 is further provided between the anode 100 and the first hole transport layer 321 so as to enhance the ability to inject holes into the first hole transport layer 321. The hole injection layer 310 may be composed of a material selected from benzidine derivatives, starburst arylamine compounds, phthalocyanine derivatives, and other materials, which is not limited in the present disclosure. The material of the hole injection layer 310 may be, for example, selected from the following compounds or any combinations thereof:

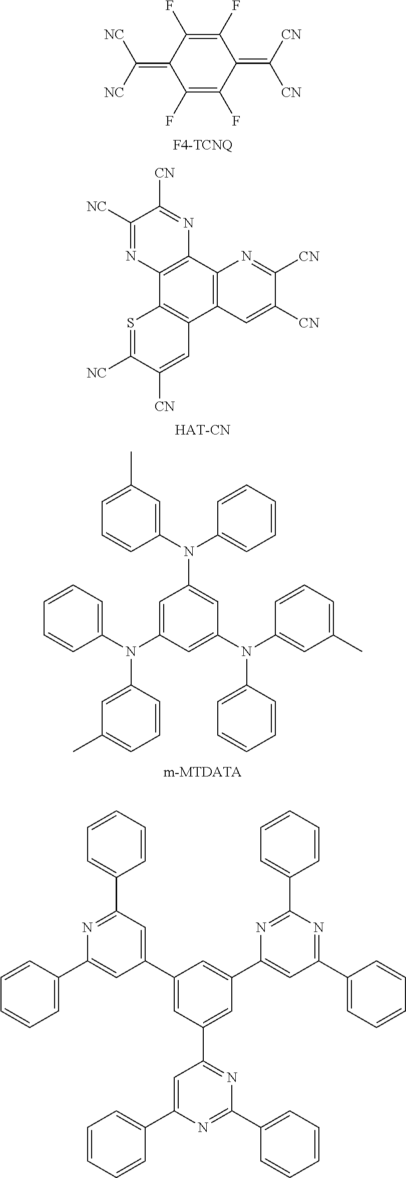

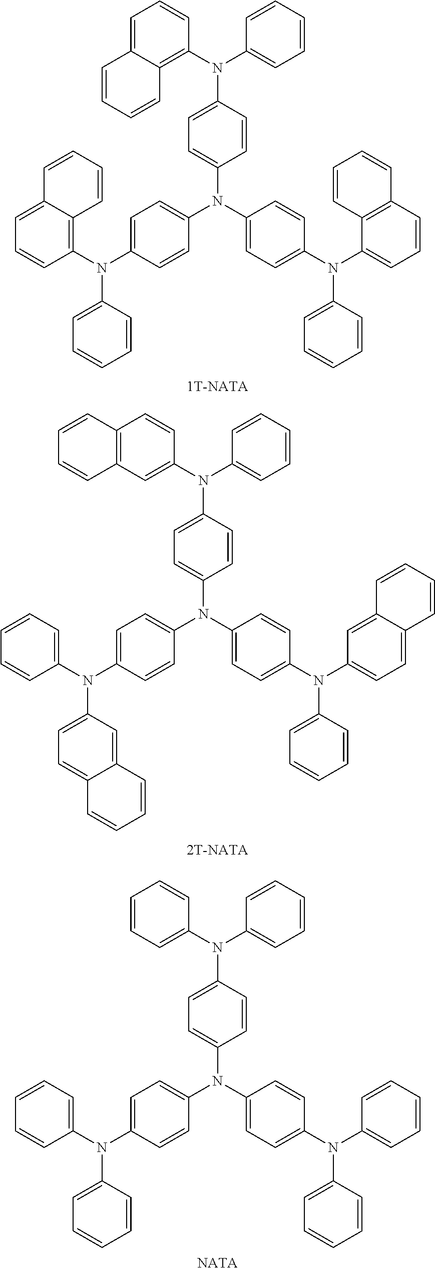

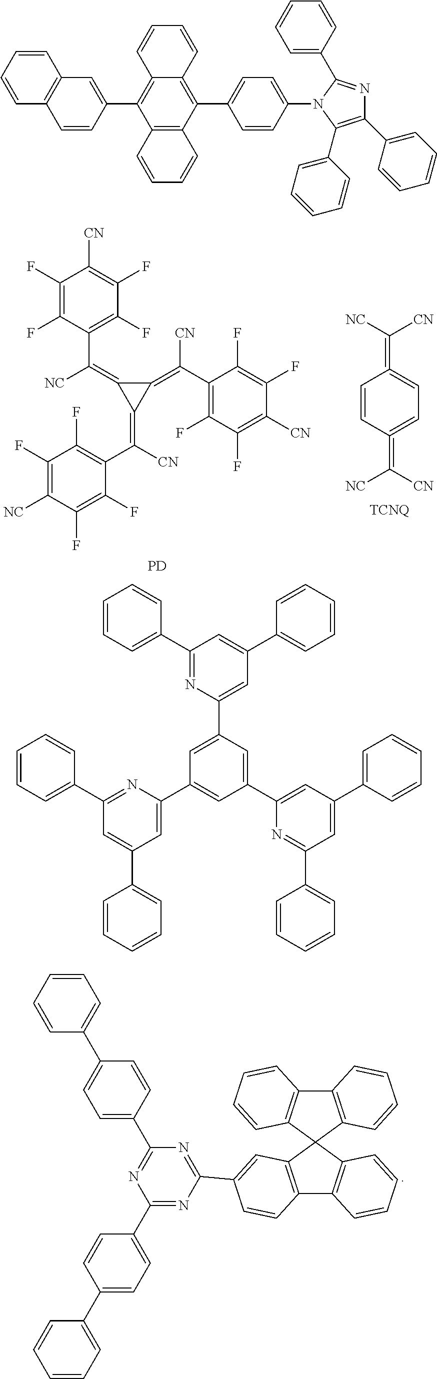

In an embodiment, the hole injection layer 310 is composed of PD, or composed of PD and HT-1.

In the present disclosure, the organic luminescent layer 330 may be composed of a single luminescent material, or may comprise a host material and a dopant material. Optionally, the organic luminescent layer 330 is composed of a host material and a dopant material. Holes injected into the organic luminescent layer 330 and electrons injected into the organic luminescent layer 330 can recombine in the organic luminescent layer 330 to form excitons. The excitons transmit energy to the host material, and the host material transmits the energy to the dopant material, thereby enabling the dopant material to emit light.

The host material of the organic luminescent layer 330 may comprise a metal chelating compound, a stilbene derivative, an aromatic amine derivative, a dibenzofuran derivative, or other types of materials. Optionally, the host material comprises the nitrogen-containing compound of the present disclosure.

The dopant material of the organic luminescent layer 330 may be a compound having a condensed aryl ring or a derivative thereof, a compound having a heteroaryl ring or a derivative thereof, an aromatic amine derivative, or other materials, which is not particularly limited in the present disclosure. The dopant material is also known as a doping material or a dopant, which can be categorized, according to its type of luminescence, as a fluorescent dopant or a phosphorescent dopant. Specific examples of the phosphorescent dopant include, but are not limited to:

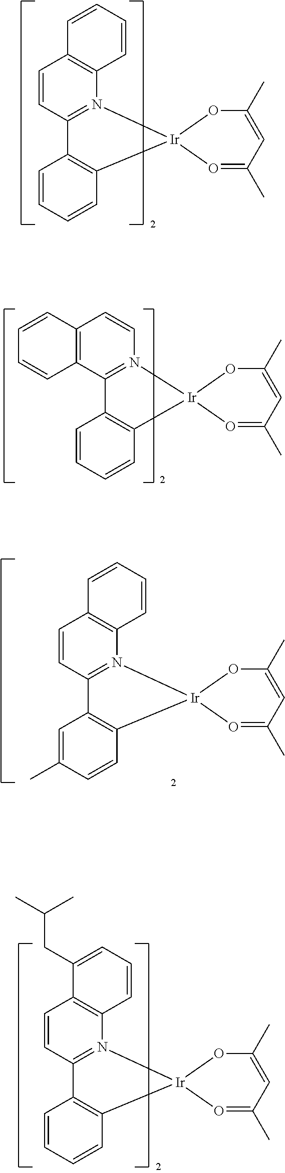

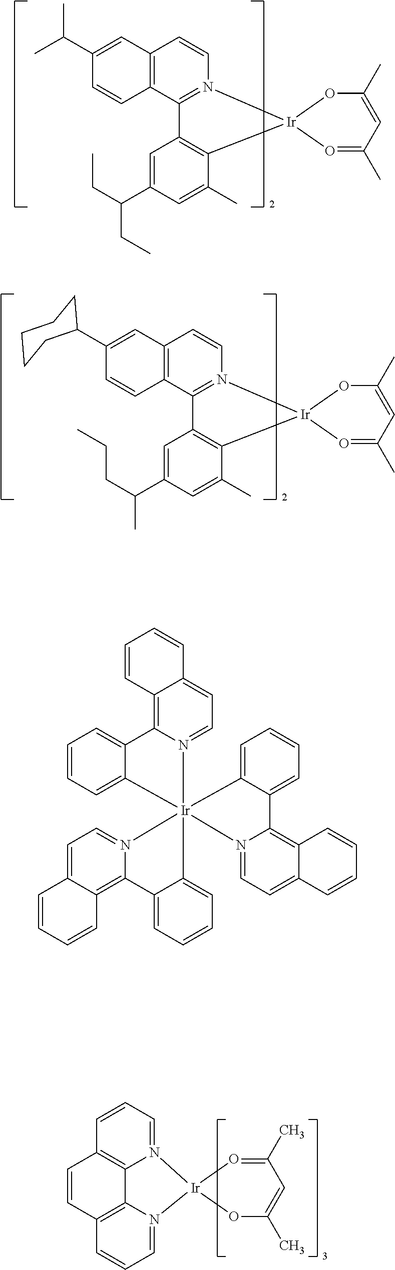

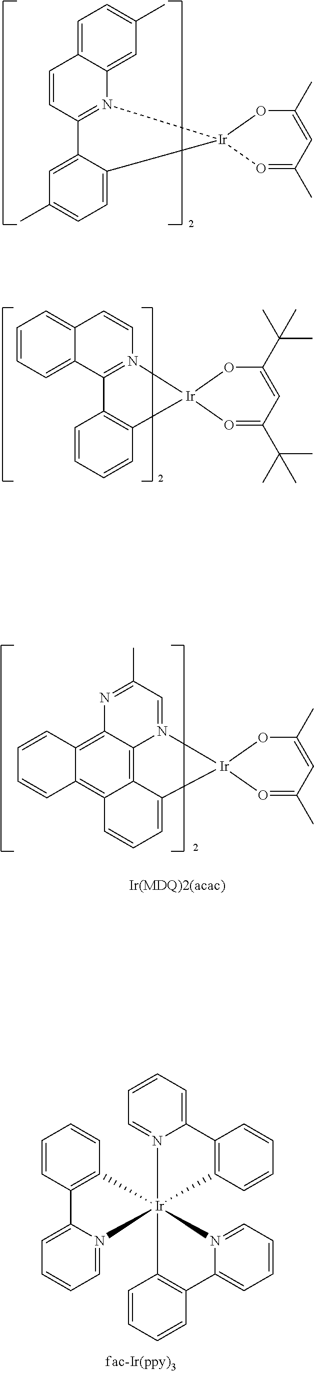

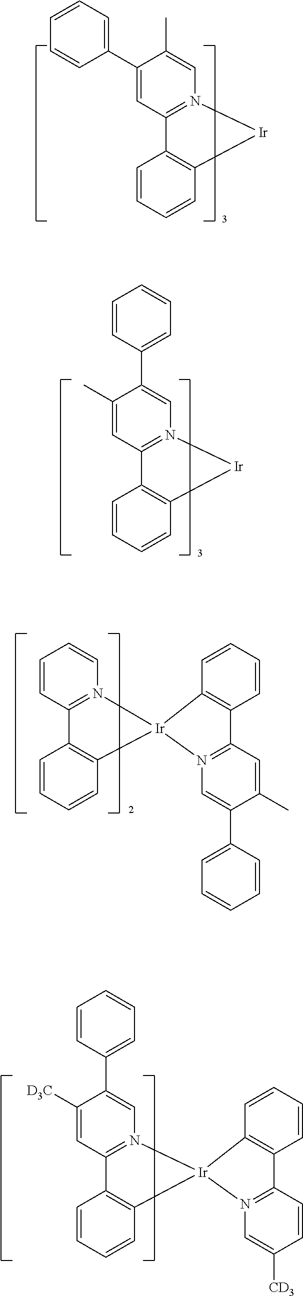

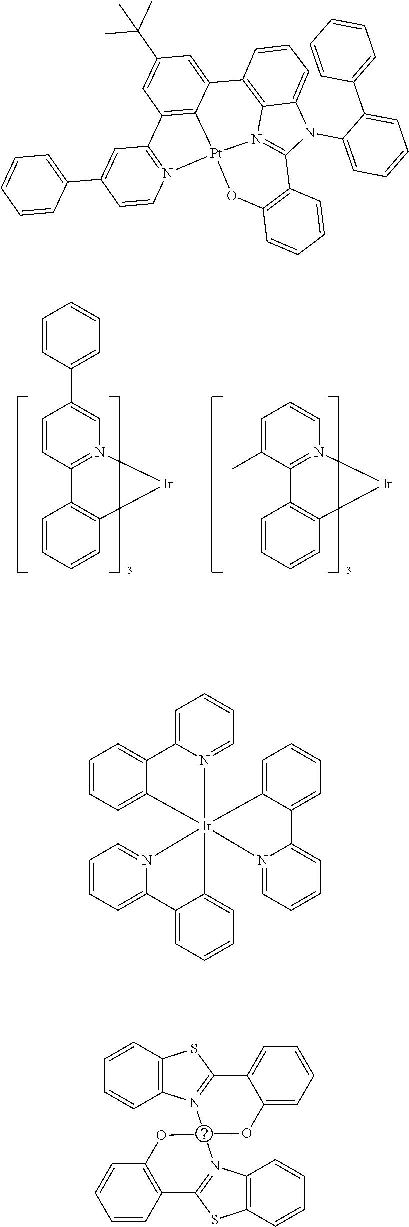

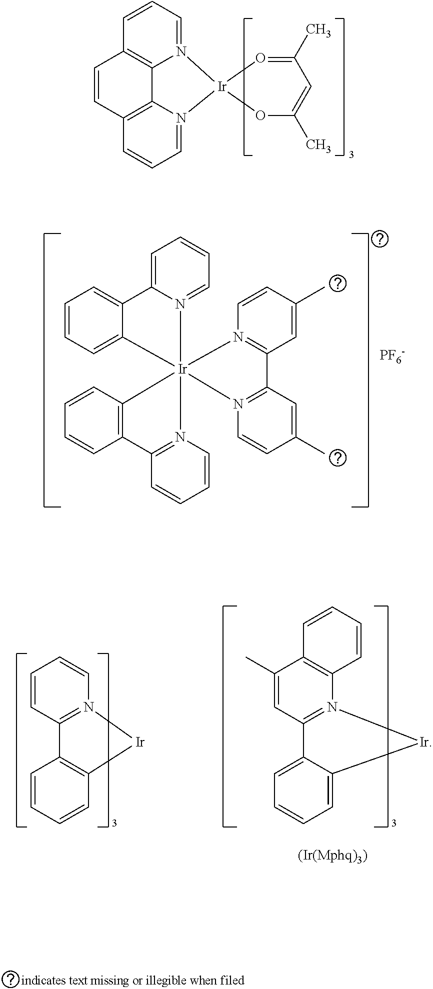

In an embodiment of the present disclosure the organic electroluminescent device is a red light-emitting organic electroluminescent device. In a more specific embodiment, the host material of the organic luminescent layer 330 comprises the nitrogen-containing compound of the present disclosure. The dopant material is, for example, Ir(Mphq)₃.

In an embodiment of the present disclosure, the organic electroluminescent device is a green light-emitting organic electroluminescent device. In a more specific embodiment, the host material of the organic luminescent layer 330 comprises the nitrogen-containing compound of the present disclosure. The dopant material is, for example, fac-Ir(ppy)₃.

The electron transport layer 340 may be a single-layer structure or a multi-layer structure, and may comprise one or more electron transport materials. The electron transport materials may be selected from, but are not limited to, BTB, LiQ, benzimidazole derivatives, oxadiazole derivatives, quinoxaline derivatives, or other electron transport materials, which are not particularly limited in the present disclosure. The material of the electron transport layer 340 includes, but is not limited to, the following compounds.

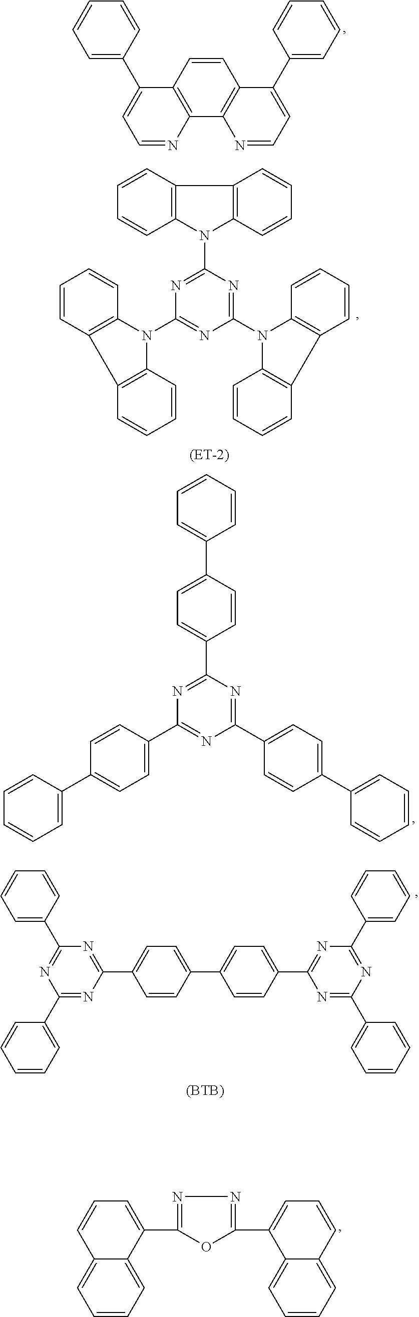

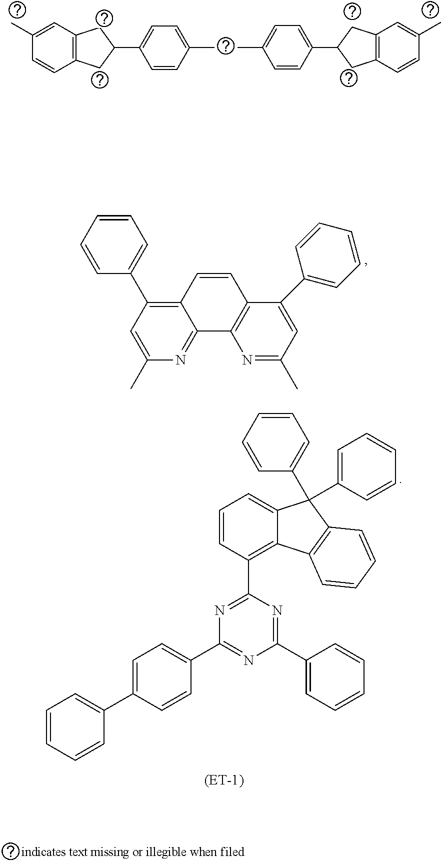

In an embodiment of the present disclosure, the electron transport layer 340 is composed of BTB and LiQ, or composed of ET-2 and LiQ.

In the present disclosure, the cathode 200 may comprise a cathode material, which is a low-work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multilayer materials such as LiF/Al, Liq/Al, LiO₂/Al, LiF/Ca, LiF/Al, and BaF₂/Ca. Optionally, a metal electrode comprising magnesium and silver is included as the cathode.

Optionally, an electron injection layer 350 is further provided between the cathode 200 and the electron transport layer 340 so as to enhance the ability to inject electrons into the electron transport layer 340. The electron injection layer 350 may comprise an inorganic material such as an alkali metal sulfide, an alkali metal halide, and the like, or may comprise a complex of an alkali metal and an organic compound. In an embodiment of the present disclosure, the electron injection layer 350 may comprise ytterbium (Yb).

The present disclosure, in a third aspect, provides an electronic apparatus including the organic electroluminescent device described in the second aspect of the present disclosure.

According to one embodiment, as shown in FIG. 2, the electronic apparatus provided is an electronic apparatus 400 including the organic electroluminescent device described above. The electronic apparatus 400 may be, for example, a display device, a lighting device, an optical communication device, or other type of electronic apparatus, including, but not limited to, for example, a computer screen, a mobile phone screen, a television, electronic paper, an emergency lamp, an optical module, etc.

A synthesis method of the nitrogen-containing compound of the present disclosure is described in detail below in conjunction with synthesis embodiments, but the present disclosure is not limited thereto in any way.

### Synthesis Embodiments

Those skilled in the art should appreciate that chemical reactions described in the present disclosure may be used properly to prepare many organic compounds of the present disclosure, and other methods that can be used to prepare the compounds of the present disclosure are all considered to be within the scope of the present disclosure. For example, the synthesis of those non-exemplary compounds of the present disclosure may be successfully accomplished by those skilled in the art by modifying the method, for example, by properly protecting an interfering group, by utilizing other known reagents other than those described in the present disclosure, or by making some conventional modifications to reaction conditions. Compounds for which a synthesis method is not mentioned in the present disclosure are raw material products obtained commercially.

When a compound involved herein is represented by both a structural formula and a CAS number and the structural formula and the CAS number conflict, the structural formula prevails.

### Synthesis of Sub-a1

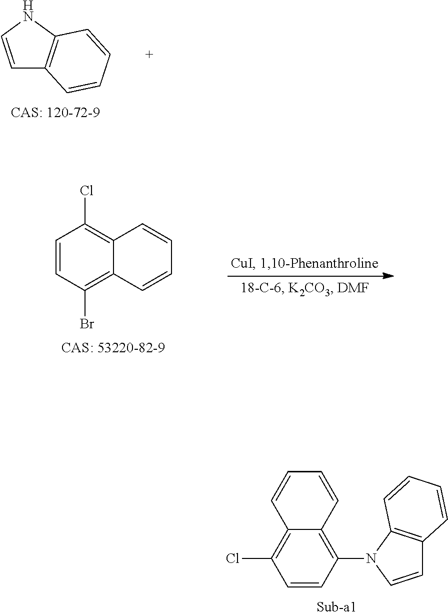 [SMILES: c1ccc2[nH]ccc2c1 ; Clc1ccc(Br)c2ccccc12 ; Clc1ccc(-n2ccc3ccccc32)c2ccccc12]

Indole (5.85 g, 50 mmol), 1-bromo-4-chloronaphthalene (13.20 g, 55 mmol), cuprous iodide (CuI, 0.19 g, 1 mmol), o-phenanthroline (3.60 g, 20 mmol), 18-crown ether-6 (1.32 g, 5 mmol), anhydrous potassium carbonate (K₂CO₃, 13.82 g, 100 mmol), and DMF (150 mL) were added sequentially to a 500-mL three-necked flask under a nitrogen atmosphere, heated to reflux and stirred for 16 hours. After being cooled to room temperature, the resulting mixture was extracted with dichloromethane (100 mL×3 times). The resulting organic phases were combined and dried over anhydrous magnesium sulfate, followed by filtration and then reduced-pressure distillation to remove the solvent, obtaining a crude product. The crude product was purified by silica gel column chromatography with n-heptane as a mobile phase, yielding white solid Sub-a1 (11.36 g, yield 82%).

Sub-a2 and Sub-a3 were synthesized respectively following the synthesis of Sub-a1, with 1-bromo-4-chloronaphthalene (CAS: 53220-82-9) being replaced with a corresponding reactant A shown in Table 1.

TABLE 1
Synthesis of Sub-a2 and Sub-a3

| Sub-a No. | Reactant A | Structure of Sub-a | Yield (%) |
|---|---|---|---|
| Sub-a2 | 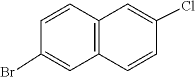 [SMILES: Clc1ccc2cc(Br)ccc2c1] | 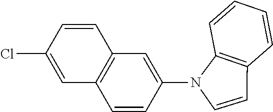 [SMILES: Clc1ccc2cc(-n3ccc4ccccc43)ccc2c1] | 84 |
| | CAS: 870822-84-7 | | |
| Sub-a3 | 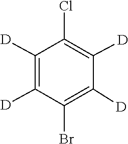 [SMILES: [2H]c1c([2H])c(Br)c([2H])c([2H])c1Cl] | 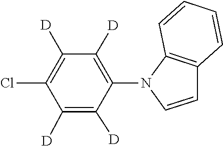 [SMILES: [2H]c1c([2H])c(-n2ccc3ccccc32)c([2H])c([2H])c1Cl] | 78 |
| | CAS: 134415-42-2 | | |

### Synthesis of Sub-b1

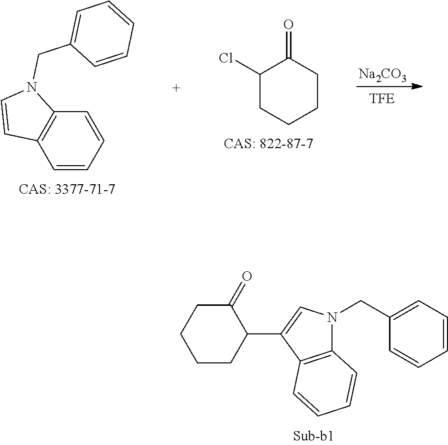

A reactant (CAS: 3377-71-7, 10.36 g, 50 mmol), 2-chlorocyclohexanone (6.60 g, 50 mmol), anhydrous sodium carbonate (Na₂CO₃, 6.36 g, 60 mmol), and 2,2,2-trifluoroacetic acid (TFE, 75 mL) were added to a 250-mL three-necked flask under a nitrogen atmosphere, and stirred at room temperature for 48 hours. After the completion of the reaction, reduced-pressure distillation was carried out to remove the solvent, obtaining a crude product. The crude product was purified by silica gel column chromatography with n-heptane as a mobile phase, yielding white solid Sub-b1 (11.97 g, yield 79%).

Sub-b2 to Sub-b12 were synthesized respectively following the synthesis of Sub-b1, with the reactant CAS: 3377-71-7 being replaced with a corresponding reactant B shown in Table 2.

TABLE 2
Synthesis of Sub-b2 to Sub-b12

| Sub-b No. | Reactant B | Structure of Sub-b | Yield (%) |
|---|---|---|---|
| Sub-b2 | 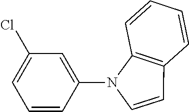 [SMILES: Clc1cccc(-n2ccc3ccccc32)c1] | 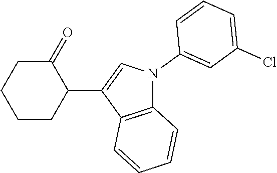 [SMILES: O=C1CCCCC1c1cn(-c2cccc(Cl)c2)c2ccccc12] | 67 |
| | CAS: 118575-85-2 | | |
| Sub-b3 | 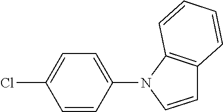 [SMILES: Clc1ccc(-n2ccc3ccccc32)cc1] | 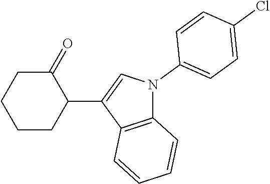 [SMILES: O=C1CCCCC1c1cn(-c2ccc(Cl)cc2)c2ccccc12] | 72 |
| | CAS: 167283-33-2 | | |
| Sub-b4 | 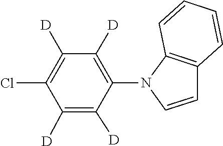 [SMILES: [2H]c1c([2H])c(-n2ccc3ccccc32)c([2H])c([2H])c1Cl] | 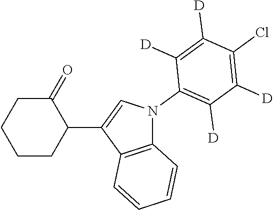 [SMILES: [2H]c1c([2H])c(-n2cc(C3CCCCC3=O)c3ccccc32)c([2H])c([2H])c1Cl] | 64 |
| | Sub-a3 | | |
| Sub-b5 | 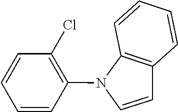 [SMILES: Clc1ccccc1-n1ccc2ccccc21] | 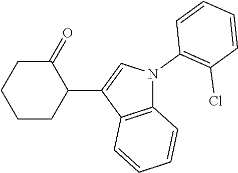 [SMILES: O=C1CCCCC1c1cn(-c2ccccc2Cl)c2ccccc12] | 61 |
| | CAS: 210162-65-5 | | |
| Sub-b6 | 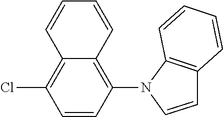 [SMILES: Clc1ccc(-n2ccc3ccccc32)c2ccccc12] | 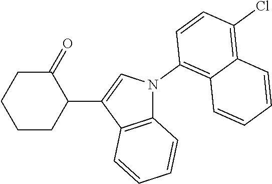 [SMILES: O=C1CCCCC1c1cn(-c2ccc(Cl)c3ccccc23)c2ccccc12] | 66 |
| | Sub-a1 | | |
| Sub-b7 | 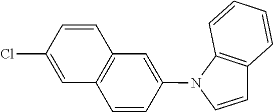 [SMILES: Clc1ccc2cc(-n3ccc4ccccc43)ccc2c1] | 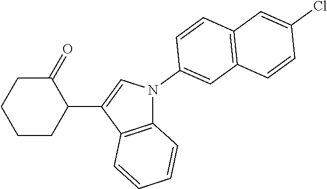 [SMILES: O=C1CCCCC1c1cn(-c2ccc3cc(Cl)ccc3c2)c2ccccc12] | 68 |
| | Sub-a2 | | |
| Sub-b8 | 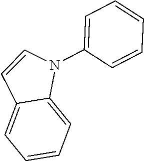 [SMILES: c1ccc(-n2ccc3ccccc32)cc1] | 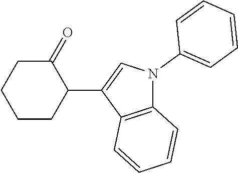 [SMILES: O=C1CCCCC1c1cn(-c2ccccc2)c2ccccc12] | 78 |
| | CAS: 16096-33-6 | | |
| Sub-b9 | 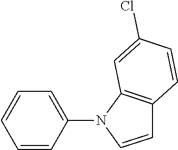 [SMILES: Clc1ccc2ccn(-c3ccccc3)c2c1] | 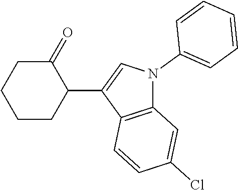 [SMILES: O=C1CCCCC1c1cn(-c2ccccc2)c2cc(Cl)ccc12] | 72 |
| | CAS: 487016-12-6 | | |
| Sub-b10 | 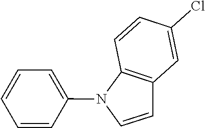 [SMILES: Clc1ccc2c(ccn2-c2ccccc2)c1] | 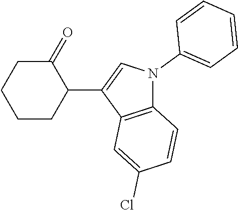 [SMILES: O=C1CCCCC1c1cn(-c2ccccc2)c2ccc(Cl)cc12] | 74 |
| | CAS: 436160-58-6 | | |
| Sub-b11 | 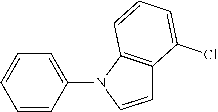 [SMILES: Clc1cccc2c1ccn2-c1ccccc1] | 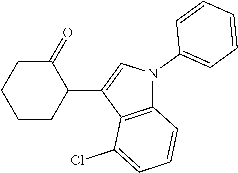 [SMILES: O=C1CCCCC1c1cn(-c2ccccc2)c2cccc(Cl)c12] | 60 |
| | CAS: 487016-12-6 | | |
| Sub-b12 | 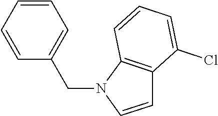 [SMILES: Clc1cccc2c1ccn2Cc1ccccc1] | 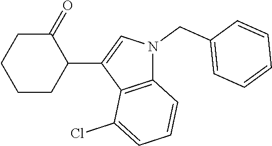 [SMILES: O=C1CCCCC1c1cn(Cc2ccccc2)c2cccc(Cl)c12] | 55 |
| | CAS: 77801-60-6 | | |

### Synthesis of Sub-c1

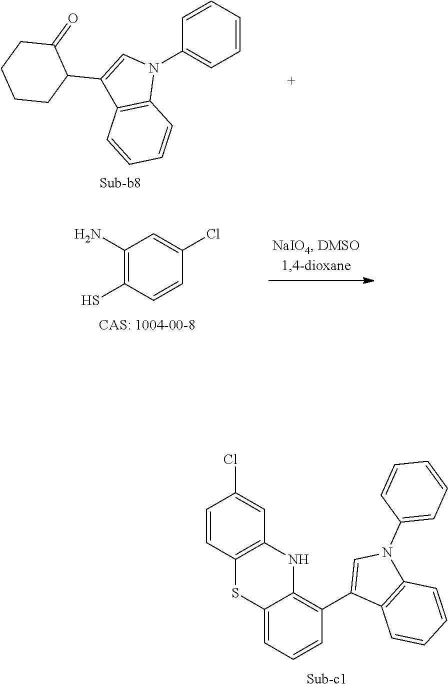 [SMILES: O=C1CCCCC1c1cn(-c2ccccc2)c2ccccc12 ; Nc1cc(Cl)ccc1S ; Clc1ccc2c(c1)Nc1c(cccc1-c1cn(-c3ccccc3)c3ccccc13)S2]

Reactant Sub-b8 (14.45 g, 50 mmol), 2-amino-4-chlorothlophenol (11.92 g, 75 mmol), sodium periodate (NaIO₄, 2.16 g, 10 mmol), DMSO (dimethyl sulfoxide, 15.63 g, 200 mmol), and 1,4-dioxane (150 mL) were added sequentially to a 500-mE three-necked flask under an air atmosphere, heated to reflux, and stirred for 12 hours. After being cooled to room temperature, the reaction solution was extracted With dichloromethane (100 mE×3 times). The resulting organic phases were combined and dried over anhydrous magnesium sulfate, followed by filtration and then reduced-pressure distillation to remove the solvent, obtaining a crude product. The crude product was purified by silica gel column chromatography with n-heptane/ethyl acetate as a mobile phase, yielding white solid Sub-c1 (8.71 g, yield 41%).

Sub-c2 to Sub-c14 were synthesized respectively following the synthesis of Sub-c1, with reactant Sub-b8 being replaced with a corresponding reactant C shown in Table 3 and with 2-amino-4-chlorothiophenol being replaced with a corresponding reactant D.

TABLE 3
Synthesis of Sub-c2 to Sub-c14

| Sub-c No. | Reactant C | Reactant D | Structure of Sub-c | Yield (%) |
|---|---|---|---|---|
| Sub-c2 | 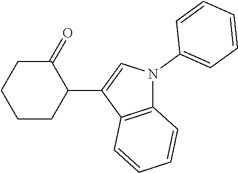 [SMILES: O=C1CCCCC1c1cn(-c2ccccc2)c2ccccc12] | 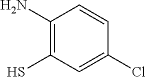 | 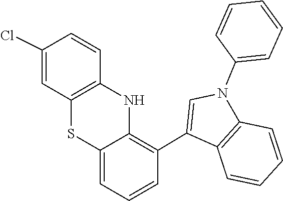 [SMILES: Clc1ccc2c(c1)Sc1cccc(-c3cn(-c4ccccc4)c4ccccc34)c1N2] | 40 |
| | Sub-b8 | CAS: 23474-98-8 | | |
| Sub-c3 | 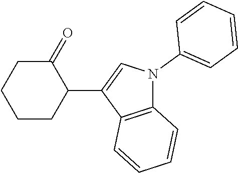 [SMILES: O=C1CCCCC1c1cn(-c2ccccc2)c2ccccc12] | 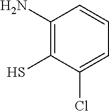 | 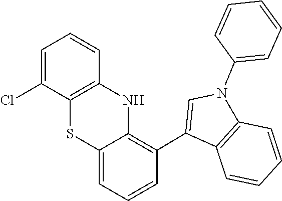 [SMILES: Clc1cccc2c1Sc1cccc(-c3cn(-c4ccccc4)c4ccccc34)c1N2] | 35 |
| | Sub-b8 | CAS: 385376-58-9 | | |
| Sub-c4 | 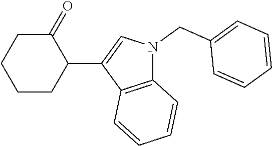 [SMILES: O=C1CCCCC1c1cn(Cc2ccccc2)c2ccccc12] | 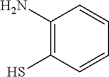 | 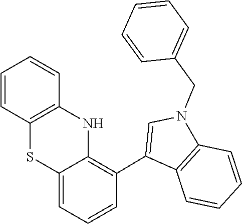 [SMILES: c1ccc(Cn2cc(-c3cccc4c3Nc3ccccc3S4)c3ccccc32)cc1] | 42 |
| | Sub-b1 | CAS: 137-07-5 | | |
| Sub-c5 | 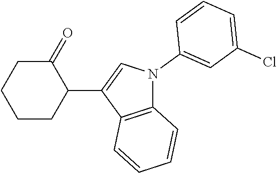 [SMILES: O=C1CCCCC1c1cn(-c2cccc(Cl)c2)c2ccccc12] | 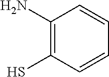 | 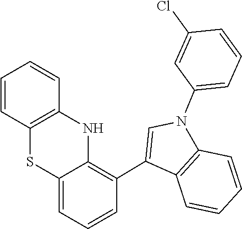 [SMILES: Clc1cccc(-n2cc(-c3cccc4c3Nc3ccccc3S4)c3ccccc32)c1] | 44 |
| | Sub-b2 | CAS: 137-07-5 | | |
| Sub-c6 | 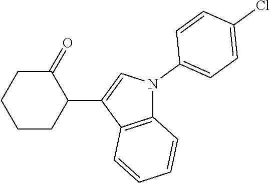 [SMILES: O=C1CCCCC1c1cn(-c2ccc(Cl)cc2)c2ccccc12] | 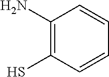 | 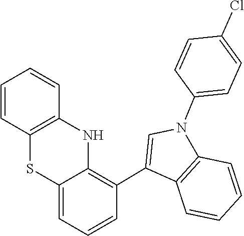 [SMILES: Clc1ccc(-n2cc(-c3cccc4c3Nc3ccccc3S4)c3ccccc32)cc1] | 35 |
| | Sub-b3 | CAS: 137-07-5 | | |
| Sub-c7 | 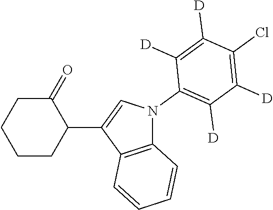 [SMILES: [2H]c1c([2H])c(-n2cc(C3CCCCC3=O)c3ccccc32)c([2H])c([2H])c1Cl] | 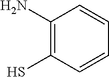 | 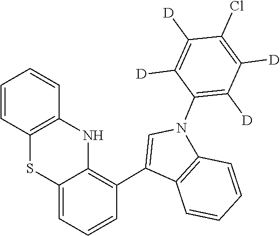 [SMILES: [2H]c1c([2H])c(-n2cc(-c3cccc4c3Nc3ccccc3S4)c3ccccc32)c([2H])c([2H])c1Cl] | 36 |
| | Sub-b4 | CAS: 137-07-5 | | |
| Sub-c8 | 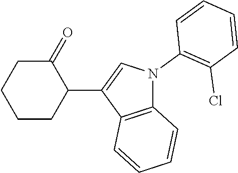 [SMILES: O=C1CCCCC1c1cn(-c2ccccc2Cl)c2ccccc12] | 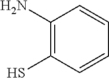 | 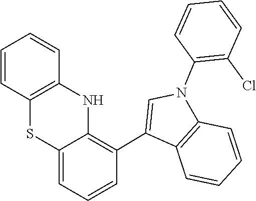 [SMILES: Clc1ccccc1-n1cc(-c2cccc3c2Nc2ccccc2S3)c2ccccc21] | 36 |
| | Sub-b5 | CAS: 137-07-5 | | |
| Sub-c9 | 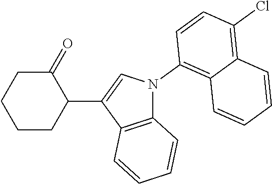 [SMILES: O=C1CCCCC1c1cn(-c2ccc(Cl)c3ccccc23)c2ccccc12] | 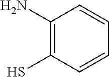 | 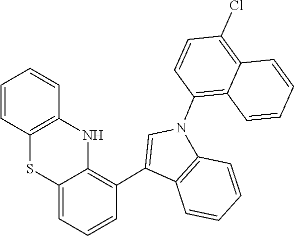 [SMILES: Clc1ccc(-n2cc(-c3cccc4c3Nc3ccccc3S4)c3ccccc32)c2ccccc12] | 39 |
| | Sub-b6 | CAS: 137-07-5 | | |
| Sub-c10 | 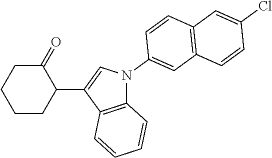 [SMILES: O=C1CCCCC1c1cn(-c2ccc3cc(Cl)ccc3c2)c2ccccc12] | 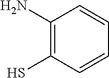 | 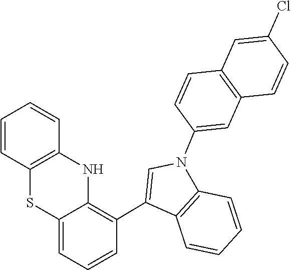 [SMILES: Clc1ccc2cc(-n3cc(-c4cccc5c4Nc4ccccc4S5)c4ccccc43)ccc2c1] | 40 |
| | Sub-b7 | CAS: 137-07-5 | | |
| Sub-c11 | 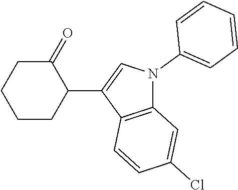 [SMILES: O=C1CCCCC1c1cn(-c2ccccc2)c2cc(Cl)ccc12] | 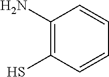 | 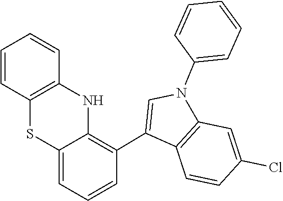 [SMILES: Clc1ccc2c(-c3cccc4c3Nc3ccccc3S4)cn(-c3ccccc3)c2c1] | 38 |
| | Sub-b9 | CAS: 137-07-5 | | |
| Sub-c12 | 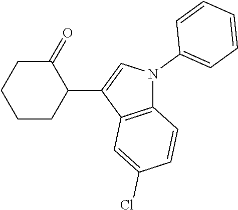 [SMILES: O=C1CCCCC1c1cn(-c2ccccc2)c2ccc(Cl)cc12] | 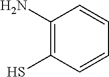 | 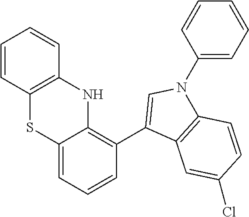 [SMILES: Clc1ccc2c(c1)c(-c1cccc3c1Nc1ccccc1S3)cn2-c1ccccc1] | 36 |
| | Sub-b10 | CAS: 137-07-5 | | |
| Sub-c13 | 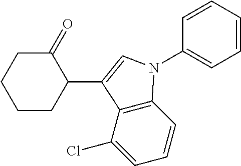 [SMILES: O=C1CCCCC1c1cn(-c2ccccc2)c2cccc(Cl)c12] | 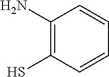 | 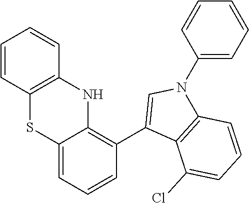 [SMILES: Clc1cccc2c1c(-c1cccc3c1Nc1ccccc1S3)cn2-c1ccccc1] | 44 |
| | Sub-b11 | CAS: 137-07-5 | | |
| Sub-c14 | 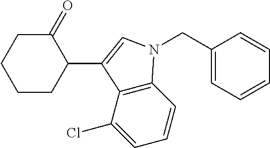 [SMILES: O=C1CCCCC1c1cn(Cc2ccccc2)c2cccc(Cl)c12] | 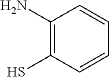 | 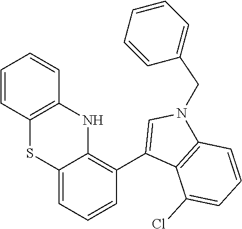 [SMILES: Clc1cccc2c1c(-c1cccc3c1Nc1ccccc1S3)cn2Cc1ccccc1] | 41 |
| | Sub-b12 | CAS: 137-07-5 | | |

### Synthesis of Sub-d1

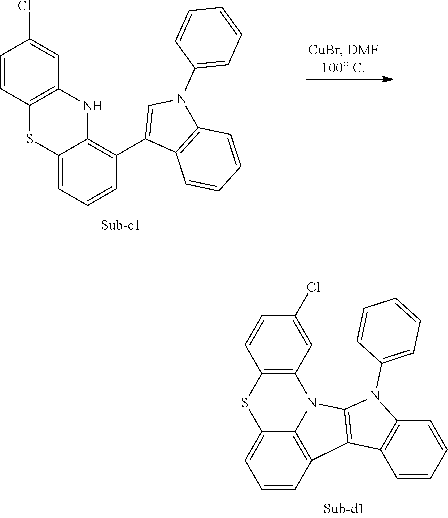 [SMILES: Clc1ccc2c(c1)Nc1c(cccc1-c1cn(-c3ccccc3)c3ccccc13)S2 ; Clc1ccc2c(c1)-n1c3c(cccc3c3c4ccccc4n(-c4ccccc4)c31)S2]

Reactant Sub-c1 (21.20 g, 50 mmol), cuprous bromide (CuBr, 1.43 g, 10 mmol), and DMF (N,N-dimethylformamide, 220 mL) were added to a 500-mL three-necked flask under an air atmosphere, heated to 100° C. and stirred for 12 hours. After being cooled to room temperature, the resulting mixture was extracted with dichloromethane (150 mL×3 times). The organic phases were combined and dried over anhydrous magnesium sulfate, followed by filtration and then reduced-pressure distillation to remove the solvent, obtaining a crude product. The crude product was purified by silica gel column chromatography with n-heptane/ethyl acetate as a mobile phase, yielding white solid Sub-d1 (14.16 g, yield 67%).

Sub-d2 to Sub-d14 were synthesized respectively following the synthesis of Sub-d1, with reactant Sub-c1 being replaced with a corresponding reactant E shown in Table 4.

TABLE 4
Synthesis of Sub-d2 to Sub-d14

| Sub-d No. | Reactant E | Structure of Sub-d | Yield (%) |
|---|---|---|---|
| Sub-d2 | 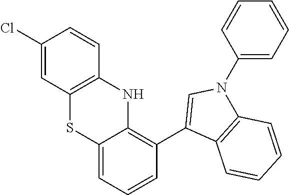 [SMILES: Clc1ccc2c(c1)Sc1cccc(-c3cn(-c4ccccc4)c4ccccc34)c1N2] | 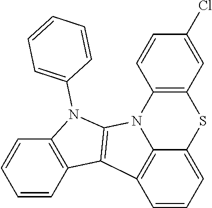 [SMILES: Clc1ccc2c(c1)Sc1cccc3c4c5ccccc5n(-c5ccccc5)c4n-2c13] | 56 |
| | Sub-c2 | | |
| Sub-d3 | 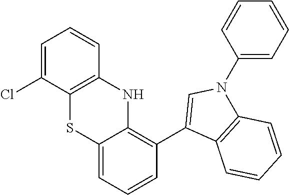 [SMILES: Clc1cccc2c1Sc1cccc(-c3cn(-c4ccccc4)c4ccccc34)c1N2] | 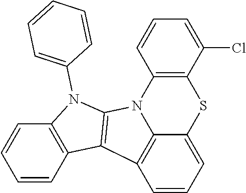 [SMILES: Clc1cccc2c1Sc1cccc3c4c5ccccc5n(-c5ccccc5)c4n-2c13] | 63 |
| | Sub-c3 | | |
| Sub-d4 | 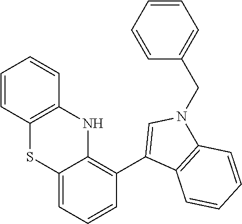 [SMILES: c1ccc(Cn2cc(-c3cccc4c3Nc3ccccc3S4)c3ccccc32)cc1] | 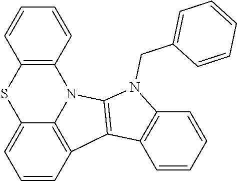 [SMILES: c1ccc(Cn2c3ccccc3c3c4cccc5c4n(c32)-c2ccccc2S5)cc1] | 56 |
| | Sub-c4 | | |
| Sub-d5 | 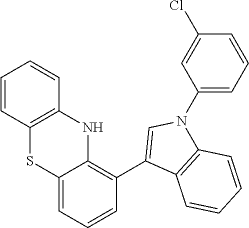 [SMILES: Clc1cccc(-n2cc(-c3cccc4c3Nc3ccccc3S4)c3ccccc32)c1] | 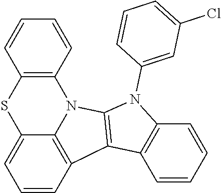 [SMILES: Clc1cccc(-n2c3ccccc3c3c4cccc5c4n(c32)-c2ccccc2S5)c1] | 63 |
| | Sub-c5 | | |
| Sub-d6 | 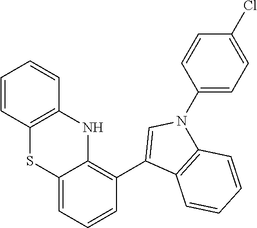 [SMILES: Clc1ccc(-n2cc(-c3cccc4c3Nc3ccccc3S4)c3ccccc32)cc1] | 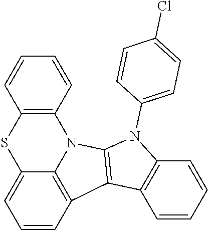 [SMILES: Clc1ccc(-n2c3ccccc3c3c4cccc5c4n(c32)-c2ccccc2S5)cc1] | 60 |
| | Sub-c6 | | |
| Sub-d7 | 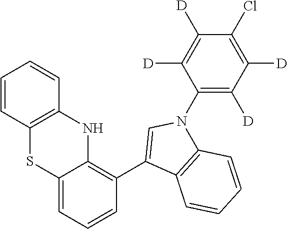 [SMILES: [2H]c1c([2H])c(-n2cc(-c3cccc4c3Nc3ccccc3S4)c3ccccc32)c([2H])c([2H])c1Cl] | 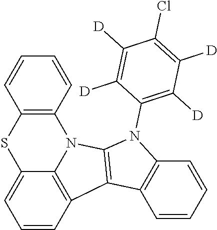 [SMILES: [2H]c1c([2H])c(-n2c3ccccc3c3c4cccc5c4n(c32)-c2ccccc2S5)c([2H])c([2H])c1Cl] | 65 |
| | Sub-c7 | | |
| Sub-d8 | 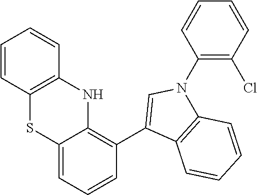 [SMILES: Clc1ccccc1-n1cc(-c2cccc3c2Nc2ccccc2S3)c2ccccc21] | 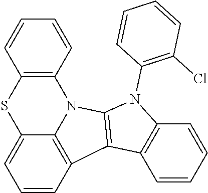 [SMILES: Clc1ccccc1-n1c2ccccc2c2c3cccc4c3n(c21)-c1ccccc1S4] | 63 |
| | Sub-c8 | | |
| Sub-d9 | 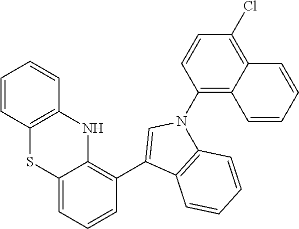 [SMILES: Clc1ccc(-n2cc(-c3cccc4c3Nc3ccccc3S4)c3ccccc32)c2ccccc12] | 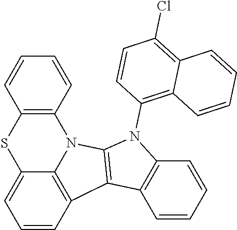 [SMILES: Clc1ccc(-n2c3ccccc3c3c4cccc5c4n(c32)-c2ccccc2S5)c2ccccc12] | 63 |
| | Sub-c9 | | |
| Sub-d10 | 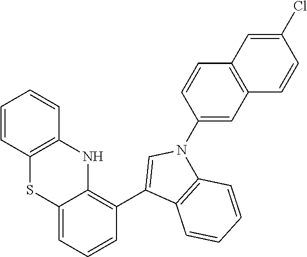 [SMILES: Clc1ccc2cc(-n3cc(-c4cccc5c4Nc4ccccc4S5)c4ccccc43)ccc2c1] | 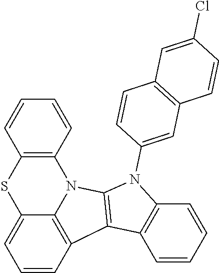 [SMILES: Clc1ccc2cc(-n3c4ccccc4c4c5cccc6c5n(c43)-c3ccccc3S6)ccc2c1] | 65 |
| | Sub-c10 | | |
| Sub-d11 | 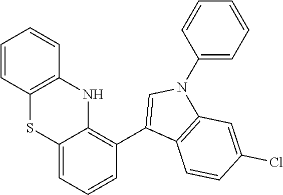 [SMILES: Clc1ccc2c(-c3cccc4c3Nc3ccccc3S4)cn(-c3ccccc3)c2c1] | 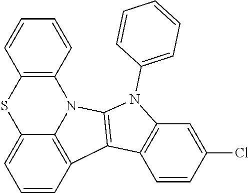 [SMILES: Clc1ccc2c3c4cccc5c4n(c3n(-c3ccccc3)c2c1)-c1ccccc1S5] | 65 |
| | Sub-c11 | | |
| Sub-d12 | 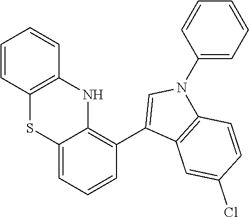 [SMILES: Clc1ccc2c(c1)c(-c1cccc3c1Nc1ccccc1S3)cn2-c1ccccc1] | 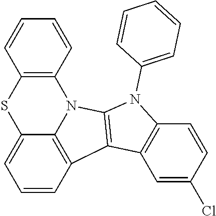 [SMILES: Clc1ccc2c(c1)c1c3cccc4c3n(c1n2-c1ccccc1)-c1ccccc1S4] | 59 |
| | Sub-c12 | | |
| Sub-d13 | 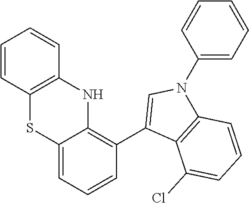 [SMILES: Clc1cccc2c1c(-c1cccc3c1Nc1ccccc1S3)cn2-c1ccccc1] | 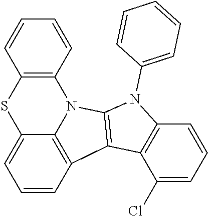 [SMILES: Clc1cccc2c1c1c3cccc4c3n(c1n2-c1ccccc1)-c1ccccc1S4] | 64 |
| | Sub-c13 | | |
| Sub-d14 | 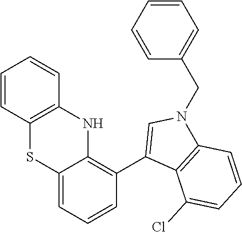 [SMILES: Clc1cccc2c1c(-c1cccc3c1Nc1ccccc1S3)cn2Cc1ccccc1] | 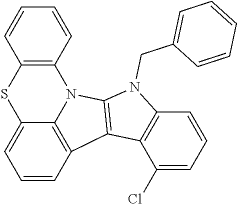 [SMILES: Clc1cccc2c1c1c3cccc4c3n(c1n2Cc1ccccc1)-c1ccccc1S4] | 63 |
| | Sub-c14 | | |

### Synthesis of Sub-e1

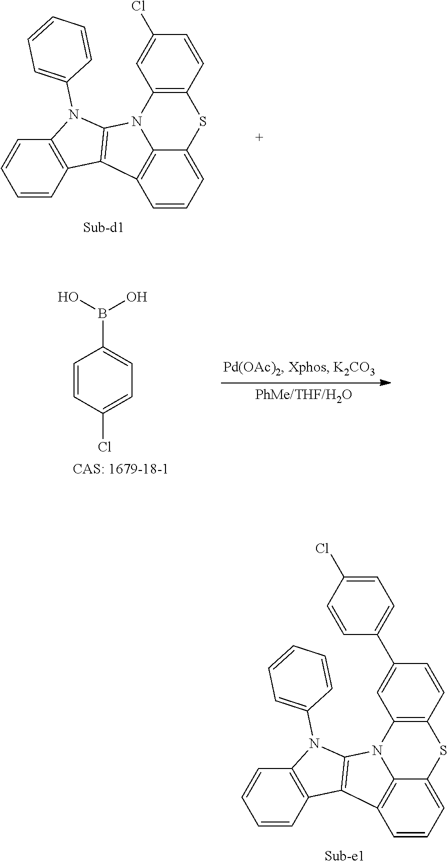 [SMILES: Clc1ccc2c(c1)-n1c3c(cccc3c3c4ccccc4n(-c4ccccc4)c31)S2 ; OB(O)c1ccc(Cl)cc1 ; Clc1ccc(-c2ccc3c(c2)-n2c4c(cccc4c4c5ccccc5n(-c5ccccc5)c42)S3)cc1]

Reactant Sub-d1 (21.10 g, 50 mmol), 4-chlorophenylboronic acid (8.58 g, 55 mmol), palladium acetate (Pd(OAc)₂, 0.22 g, 1.0 mmol), 2-dicyclohexylphosphino-2′,4′,6′-triisopropylbiphenyl (X-phos, 0.95 g, 2 mmol), anhydrous potassium carbonate (K₂CO₃, 13.82 g, 100 mmol), toluene (PhMe, 220 mL), tetrahydrofuran (THF, 55 mL), and deionized water (H₂O, 55 mL) were added sequentially to a 500-mL three-necked flask under a nitrogen atmosphere, heated to reflux and stirred for 16 hours. After being cooled to room temperature, the resulting mixture was extracted with dichloromethane (100 mL×3 times). The separated organic phases were combined and dried over anhydrous magnesium sulfate, followed by filtration and then reduced-pressure distillation to remove the solvent, obtaining a crude product. The crude product was purified by silica gel column chromatography with n-heptane as a mobile phase, yielding white solid Sub-e1 (18.18 g, yield 73%).

Sub-e2 was synthesized following the synthesis of Sub-e1, with 4-chlorophenylboronic acid (CAS: 1679-18-1) being replaced with reactant F shown in Table 5.

TABLE 5
Synthesis of Sub-e2

| Sub-e No. | Reactant F | Structure of Sub-e | Yield (%) |
|---|---|---|---|
| Sub-e2 | 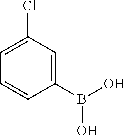 | 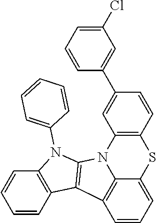 [SMILES: Clc1cccc(-c2ccc3c(c2)-n2c4c(cccc4c4c5ccccc5n(-c5ccccc5)c42)S3)c1] | 75 |
| | CAS: 63503-60-6 | | |

### Synthesis of Sub-f1

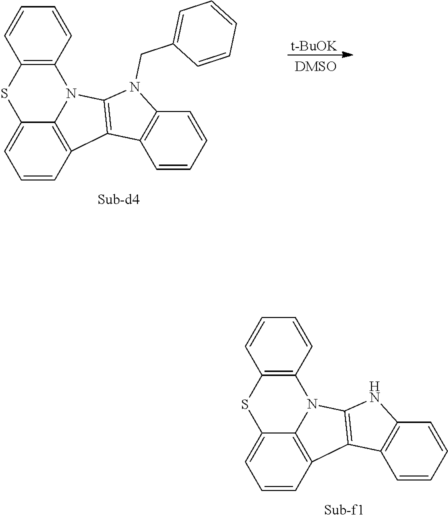 [SMILES: "t-BuOK" ; c1ccc(Cn2c3ccccc3c3c4cccc5c4n(c32)-c2ccccc2S5)cc1 ; c1ccc2c(c1)Sc1cccc3c4c5ccccc5[nH]c4n-2c13]

Reactant Sub-d4 (20.11 g, 50 mmol), potassium tert-butoxide (t-BuOK, 56.10 g, 500 mmol), and DMSO (dimethyl sulfoxide, 300 mL) were added sequentially to a 500-mL three-necked flask under a nitrogen atmosphere, heated to 50° C.-60° C. and stirred for 4 hours. After the reaction mixture was cooled to room temperature, the reaction solution was poured into 500 mL of deionized water, and a precipitate was formed. The precipitate was filtered and the resulting solid was collected. The solid was dissolved in dichloromethane (200 mL), dried by adding anhydrous sodium sulfate, and filtered. The resulting filtrate was collected and distilled under reduced pressure to remove the solvent, obtaining a crude product. The crude product was purified by silica column chromatography with n-heptane/dichloromethane as a mobile phase, yielding white solid Compound Sub-f1 (13.11 g, yield 84%).

Sub-f2 was synthesized following the synthesis of Sub-f1, with reactant Sub-d4 being replaced with reactant G shown in Table 6.

TABLE 6
Synthesis of Sub-f2

| Sub-f No. | Reactant G | Structure of Sub-f | Yield (%) |
|---|---|---|---|
| Sub-f2 | 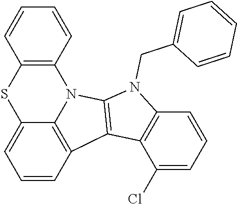 [SMILES: Clc1cccc2c1c1c3cccc4c3n(c1n2Cc1ccccc1)-c1ccccc1S4] | 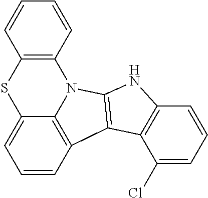 [SMILES: Clc1cccc2[nH]c3c(c4cccc5c4n3-c3ccccc3S5)c12] | 75 |
| | Sub-d14 | | |

### Synthesis of Sub-g1

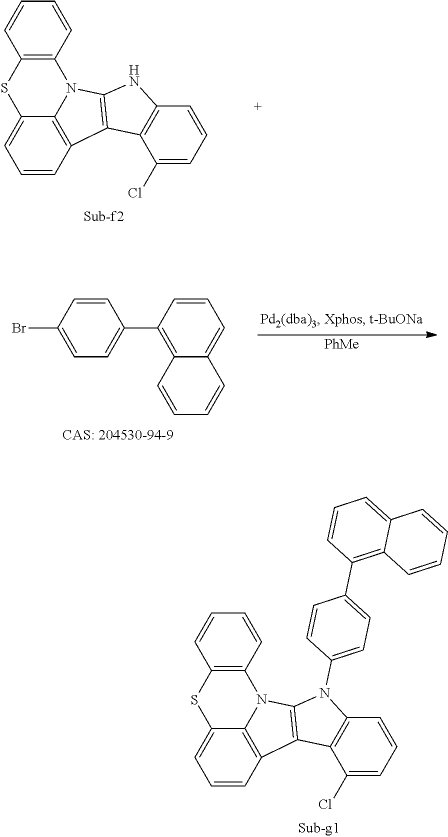 [SMILES: Clc1cccc2[nH]c3c(c4cccc5c4n3-c3ccccc3S5)c12 ; Brc1ccc(-c2cccc3ccccc23)cc1 ; "PhMe" ; Clc1cccc2c1c1c3cccc4c3n(c1n2-c1ccc(-c2cccc3ccccc23)cc1)-c1ccccc1S4]

Reactant Sub-f2 (17.30 g, 50 mmol), 1-(4-bromophenyl)naphthalene (15.51 g, 55 mmol), tris(dibenzylideneacetone)dipalladium (Pd₂(dba)₃, 0.916 g, 1 mmol), XPhos (0.95 g, 2 mmol), tert-butanoate sodium (t-BuONa, 9.61 g, 100 mmol), and toluene (PhMe, 180 mmol) were added sequentially to a 500-mL three-necked flask under a nitrogen atmosphere. The mixture was heated to reflux and stirred overnight. After being cooled to room temperature, the reaction solution was poured into 500 mL of deionized water, stirred thoroughly for 30 minutes, and filtered. The resulting filter cake was rinsed with deionized water to neutral, and then rinsed with absolute ethanol (200 mL), obtaining a crude product. The crude product was purified by silica gel column chromatography with n-heptane/dichloromethane as a mobile phase, yielding white solid Sub-g1 (20.0 g, yield 73%).

Sub-g2 was synthesized following the synthesis of Sub-g1, with reactant Sub-f2 being replaced with reactant H shown in Table 7 and with 1-(4-bromophenyl)naphthalene (CAS: 204530-94-9) being replaced with reactant J.

TABLE 7
Synthesis of Sub-g2

| Sub-g No. | Reactant H | Reactant J | Structure of Sub-g | Yield (%) |
|---|---|---|---|---|
| Sub-g2 | 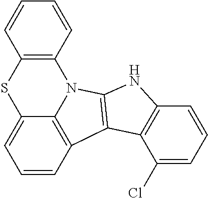 [SMILES: Clc1cccc2[nH]c3c(c4cccc5c4n3-c3ccccc3S5)c12] | 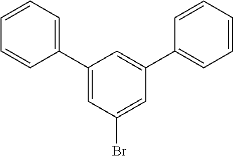 [SMILES: Brc1cc(-c2ccccc2)cc(-c2ccccc2)c1] | 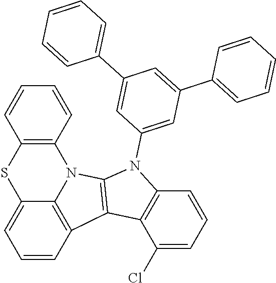 [SMILES: Clc1cccc2c1c1c3cccc4c3n(c1n2-c1cc(-c2ccccc2)cc(-c2ccccc2)c1)-c1ccccc1S4] | 64 |
| | Sub-f2 | CAS: 103068-20-8 | | |

### Synthesis of Sub-h1

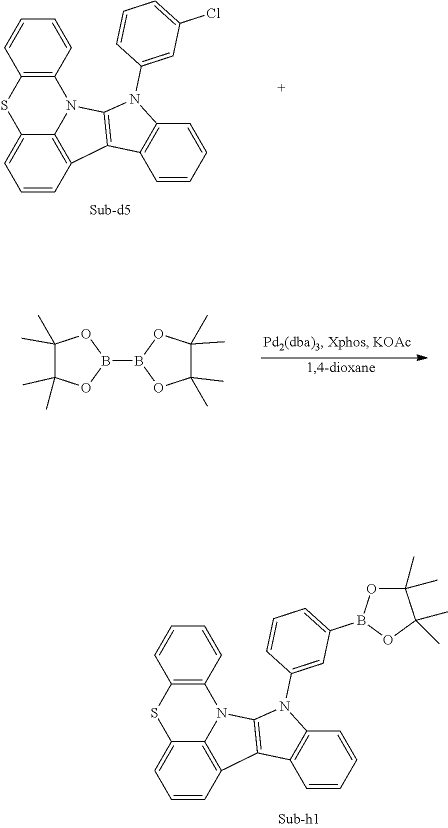

Reactant Sub-d5 (21.10 g, 50 mmol), bis(pinacolato)diboron (15.24 g, 60 mmol), potassium acetate (KOAc, 9.81 g, 100 mmol), and 1,4-dioxane (220 mL) were added sequentially to a 500-mL three-necked flask under a nitrogen atmosphere, stirred and heated. Once the resulting mixture was heated to 40° C., Pd₂(dba)₃ (tris(dibenzylideneacetone)dipalladium, 0.46 g, 0.5 mmol), and XPhos (0.48 g, 1.0 mmol) were quickly added, followed by heating to reflux, and stirred overnight. After the reaction solution was cooled to room temperature, 200 mL of water was added thereto. The resulting mixture was stirred thoroughly for 30 minutes, and filtered under reduced pressure. The resulting filter cake was washed with deionized water to neutral, and then rinsed with 100 mL of absolute ethanol, obtaining a gray solid crude product. The crude product was beaten with n-heptane once, dissolved in 200 mL of toluene, passed through a silica gel column to remove the catalyst, and then concentrated, yielding white solid Sub-h1 (19.54 g, yield 76%).

Sub-h2 to Sub-h16 were synthesized respectively following the synthesis of Sub-h1, with reactant Sub-d5 being replaced with a corresponding reactant K shown in Table 8.

TABLE 8
Synthesis of Sub-h2 to Sub-h16

| Sub-h No. | Reactant K | Structure of Sub-h | Yield (%) |
|---|---|---|---|
| Sub-h2 | 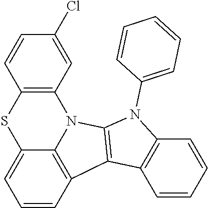 [SMILES: Clc1ccc2c(c1)-n1c3c(cccc3c3c4ccccc4n(-c4ccccc4)c31)S2] | 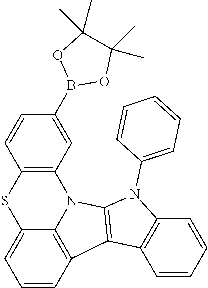 [SMILES: CC1(C)OB(c2ccc3c(c2)-n2c4c(cccc4c4c5ccccc5n(-c5ccccc5)c42)S3)OC1(C)C] | 79 |
| | Sub-d1 | | |
| Sub-h3 | 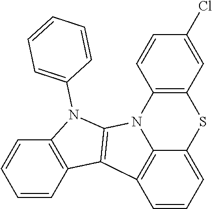 [SMILES: Clc1ccc2c(c1)Sc1cccc3c4c5ccccc5n(-c5ccccc5)c4n-2c13] | 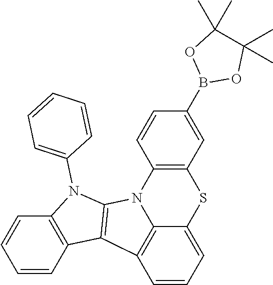 [SMILES: CC1(C)OB(c2ccc3c(c2)Sc2cccc4c5c6ccccc6n(-c6ccccc6)c5n-3c24)OC1(C)C] | 70 |
| | Sub-d2 | | |
| Sub-h4 | 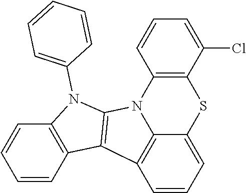 [SMILES: Clc1cccc2c1Sc1cccc3c4c5ccccc5n(-c5ccccc5)c4n-2c13] | 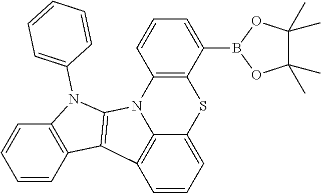 [SMILES: CC1(C)OB(c2cccc3c2Sc2cccc4c5c6ccccc6n(-c6ccccc6)c5n-3c24)OC1(C)C] | 66 |
| | Sub-d3 | | |
| Sub-h5 | 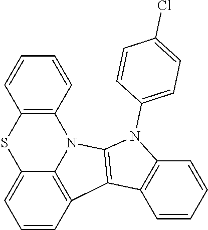 [SMILES: Clc1ccc(-n2c3ccccc3c3c4cccc5c4n(c32)-c2ccccc2S5)cc1] | 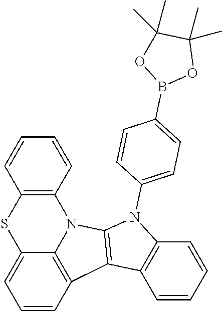 [SMILES: CC1(C)OB(c2ccc(-n3c4ccccc4c4c5cccc6c5n(c43)-c3ccccc3S6)cc2)OC1(C)C] | 75 |
| | Sub-d6 | | |
| Sub-h6 | 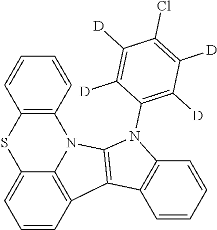 [SMILES: [2H]c1c([2H])c(-n2c3ccccc3c3c4cccc5c4n(c32)-c2ccccc2S5)c([2H])c([2H])c1Cl] | 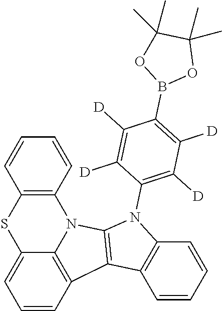 [SMILES: [2H]c1c([2H])c(-n2c3ccccc3c3c4cccc5c4n(c32)-c2ccccc2S5)c([2H])c([2H])c1B1OC(C)(C)C(C)(C)O1] | 73 |
| | Sub-d7 | | |
| Sub-h7 | 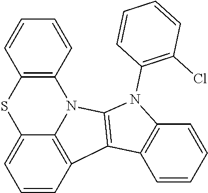 [SMILES: Clc1ccccc1-n1c2ccccc2c2c3cccc4c3n(c21)-c1ccccc1S4] | 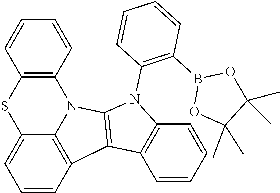 [SMILES: CC1(C)OB(c2ccccc2-n2c3ccccc3c3c4cccc5c4n(c32)-c2ccccc2S5)OC1(C)C] | 60 |
| | Sub-d8 | | |
| Sub-h8 | 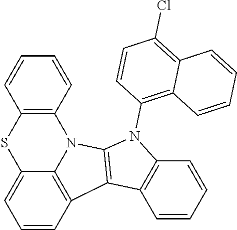 [SMILES: Clc1ccc(-n2c3ccccc3c3c4cccc5c4n(c32)-c2ccccc2S5)c2ccccc12] | 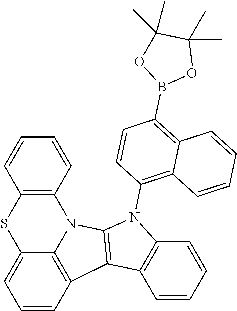 [SMILES: CC1(C)OB(c2ccc(-n3c4ccccc4c4c5cccc6c5n(c43)-c3ccccc3S6)c3ccccc23)OC1(C)C] | 70 |
| | Sub-d9 | | |
| Sub-h9 | 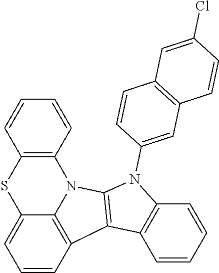 [SMILES: Clc1ccc2cc(-n3c4ccccc4c4c5cccc6c5n(c43)-c3ccccc3S6)ccc2c1] | 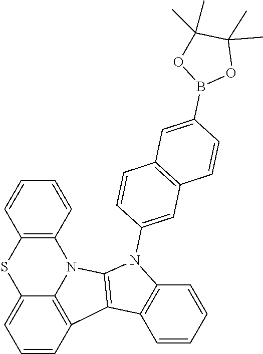 [SMILES: CC1(C)OB(c2ccc3cc(-n4c5ccccc5c5c6cccc7c6n(c54)-c4ccccc4S7)ccc3c2)OC1(C)C] | 72 |
| | Sub-d10 | | |
| Sub-h10 | 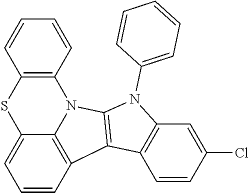 [SMILES: Clc1ccc2c3c4cccc5c4n(c3n(-c3ccccc3)c2c1)-c1ccccc1S5] | 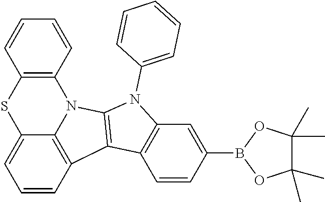 [SMILES: CC1(C)OB(c2ccc3c4c5cccc6c5n(c4n(-c4ccccc4)c3c2)-c2ccccc2S6)OC1(C)C] | 77 |
| | Sub-d11 | | |
| Sub-h11 | 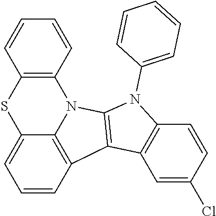 [SMILES: Clc1ccc2c(c1)c1c3cccc4c3n(c1n2-c1ccccc1)-c1ccccc1S4] | 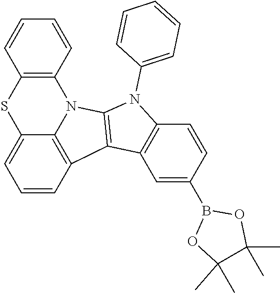 [SMILES: CC1(C)OB(c2ccc3c(c2)c2c4cccc5c4n(c2n3-c2ccccc2)-c2ccccc2S5)OC1(C)C] | 78 |
| | Sub-d12 | | |
| Sub-h12 | 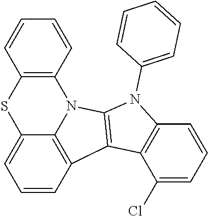 [SMILES: Clc1cccc2c1c1c3cccc4c3n(c1n2-c1ccccc1)-c1ccccc1S4] | 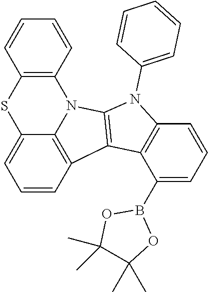 [SMILES: CC1(C)OB(c2cccc3c2c2c4cccc5c4n(c2n3-c2ccccc2)-c2ccccc2S5)OC1(C)C] | 64 |
| | Sub-d13 | | |
| Sub-h13 | 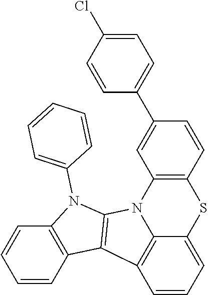 [SMILES: Clc1ccc(-c2ccc3c(c2)-n2c4c(cccc4c4c5ccccc5n(-c5ccccc5)c42)S3)cc1] | 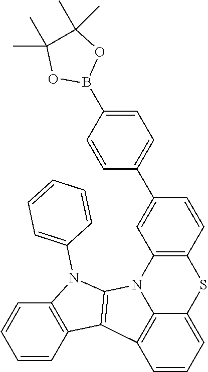 [SMILES: CC1(C)OB(c2ccc(-c3ccc4c(c3)-n3c5c(cccc5c5c6ccccc6n(-c6ccccc6)c53)S4)cc2)OC1(C)C] | 76 |
| | Sub-e1 | | |
| Sub-h14 | 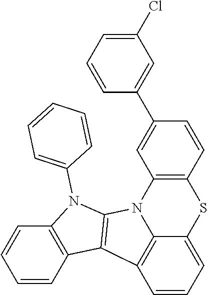 [SMILES: Clc1cccc(-c2ccc3c(c2)-n2c4c(cccc4c4c5ccccc5n(-c5ccccc5)c42)S3)c1] | 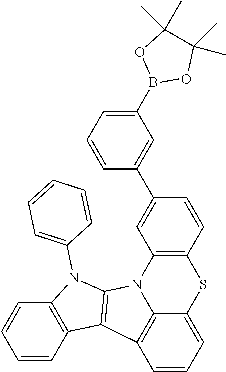 [SMILES: CC1(C)OB(c2cccc(-c3ccc4c(c3)-n3c5c(cccc5c5c6ccccc6n(-c6ccccc6)c53)S4)c2)OC1(C)C] | 73 |
| | Sub-e2 | | |
| Sub-h15 | 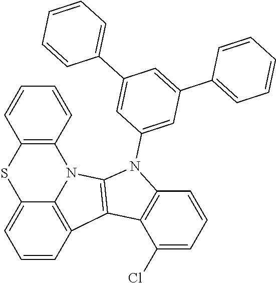 [SMILES: Clc1cccc2c1c1c3cccc4c3n(c1n2-c1cc(-c2ccccc2)cc(-c2ccccc2)c1)-c1ccccc1S4] | 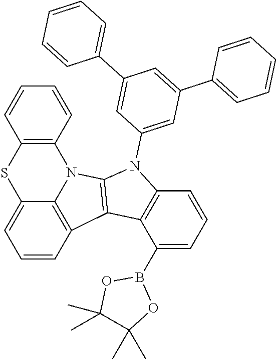 [SMILES: CC1(C)OB(c2cccc3c2c2c4cccc5c4n(c2n3-c2cc(-c3ccccc3)cc(-c3ccccc3)c2)-c2ccccc2S5)OC1(C)C] | 62 |
| | Sub-g2 | | |
| Sub-h16 | 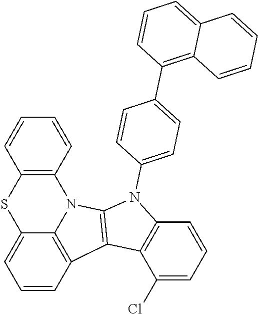 [SMILES: Clc1cccc2c1c1c3cccc4c3n(c1n2-c1ccc(-c2cccc3ccccc23)cc1)-c1ccccc1S4] | 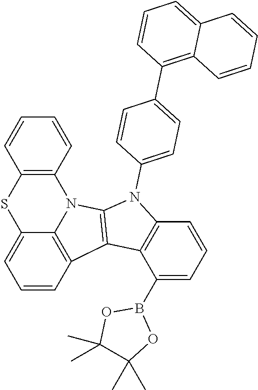 [SMILES: CC1(C)OB(c2cccc3c2c2c4cccc5c4n(c2n3-c2ccc(-c3cccc4ccccc34)cc2)-c2ccccc2S5)OC1(C)C] | 64 |
| | Sub-g1 | | |

### Synthesis of Sub-j1

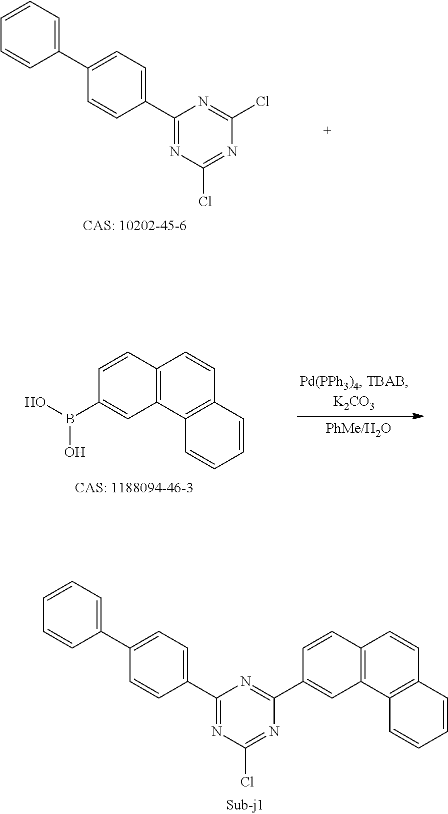 [SMILES: Clc1nc(Cl)nc(-c2ccc(-c3ccccc3)cc2)n1 ; OB(O)c1ccc2ccc3ccccc3c2c1 ; Clc1nc(-c2ccc(-c3ccccc3)cc2)nc(-c2ccc3ccc4ccccc4c3c2)n1]

2-(4-biphenyl)-4,6-dichloro-1,3,5-triazine (22.66 g, 75 mmol), 3-phenanthroboronic acid (11.10 g, 50 mmol), tetrakis(triphenylphosphine)palladium (Pd(PPh₃)₄, 0.58 g, 0.5 mmol), tetrabutylammonium bromide (TBAB, 1.61 g, 5 mmol), anhydrous potassium carbonate (K₂CO₃, 13.82 g, 100 mmol), toluene (PhMe, 220 mL), and deionized water (H₂O, 55 mL) were added sequentially to a 500-mL three-necked flask under a nitrogen atmosphere, heated to 65° C.-70° C. and stirred for 16 hours. After being cooled to room temperature, the reaction solution was extracted with dichloromethane (100 mL×3 times). The resulting organic phase was dried over anhydrous magnesium sulfate, filtered and distilled under reduced pressure to remove the solvent, obtaining a crude product. The crude product was recrystallized with toluene, yielding white solid Sub-j1 (14.62 g, yield 66%).

Sub-j2 to SubHj6 were synthesized respectively following the synthesis of Sub-j1, with 2-(4-biphenyl)-4,6-dichloro-1,3,5-triazine (CASS: 10202-45-6) being replaced with a corresponding reactant L shown in Table 9 and with 3-phenanthroboronic acid (CAS: 1188094-46-3) being replaced with a corresponding reactant M.

TABLE 9
Synthesis of Sub-j2 to Sub-j6

| Sub-j No. | Reactant L | Reactant M | Structure of Sub-j | Yield (%) |
|---|---|---|---|---|
| Sub-j2 | 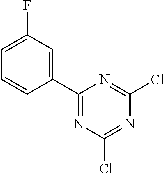 [SMILES: Fc1cccc(-c2nc(Cl)nc(Cl)n2)c1] | 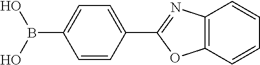 [SMILES: OB(O)c1ccc(-c2nc3ccccc3o2)cc1] | 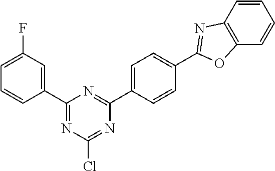 [SMILES: Fc1cccc(-c2nc(Cl)nc(-c3ccc(-c4nc5ccccc5o4)cc3)n2)c1] | 63 |
| | CAS: 102528-19-8 | CAS: 1065657-51-3 | | |
| Sub-j3 | 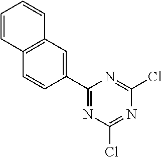 [SMILES: Clc1nc(Cl)nc(-c2ccc3ccccc3c2)n1] | 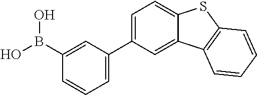 [SMILES: OB(O)c1cccc(-c2ccc3sc4ccccc4c3c2)c1] | 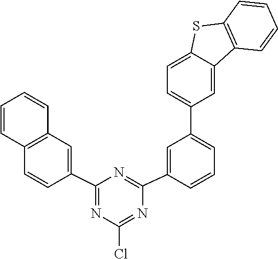 [SMILES: Clc1nc(-c2cccc(-c3ccc4sc5ccccc5c4c3)c2)nc(-c2ccc3ccccc3c2)n1] | 55 |
| | CAS: 112719-97-8 | CAS: 1172134-22-3 | | |
| Sub-j4 | 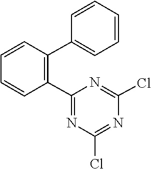 [SMILES: Clc1nc(Cl)nc(-c2ccccc2-c2ccccc2)n1] | 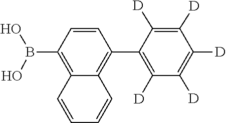 [SMILES: [2H]c1c([2H])c([2H])c(-c2ccc(B(O)O)c3ccccc23)c([2H])c1[2H]] | 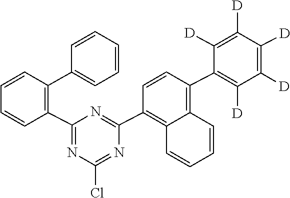 [SMILES: [2H]c1c([2H])c([2H])c(-c2ccc(-c3nc(Cl)nc(-c4ccccc4-c4ccccc4)n3)c3ccccc23)c([2H])c1[2H]] | 61 |
| | CAS: 2134165-04-9 | CAS: 1315551-74-6 | | |
| Sub-j5 | 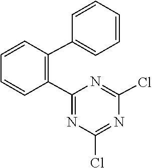 [SMILES: Clc1nc(Cl)nc(-c2ccccc2-c2ccccc2)n1] | 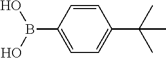 | 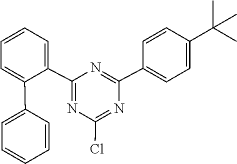 [SMILES: CC(C)(C)c1ccc(-c2nc(Cl)nc(-c3ccccc3-c3ccccc3)n2)cc1] | 65 |
| | CAS: 2134165-04-9 | CAS: 123324-71-0 | | |
| Sub-j6 | 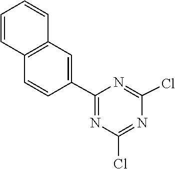 [SMILES: Clc1nc(Cl)nc(-c2ccc3ccccc3c2)n1] | 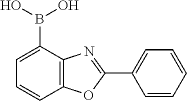 [SMILES: OB(O)c1cccc2oc(-c3ccccc3)nc12] | 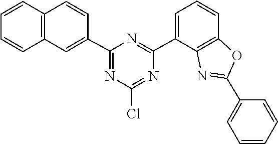 [SMILES: Clc1nc(-c2ccc3ccccc3c2)nc(-c2cccc3oc(-c4ccccc4)nc23)n1] | 60 |
| | CAS: 112719-97-8 | CAS: 2148916-29-2 | | |

### Synthesis of Compound 3

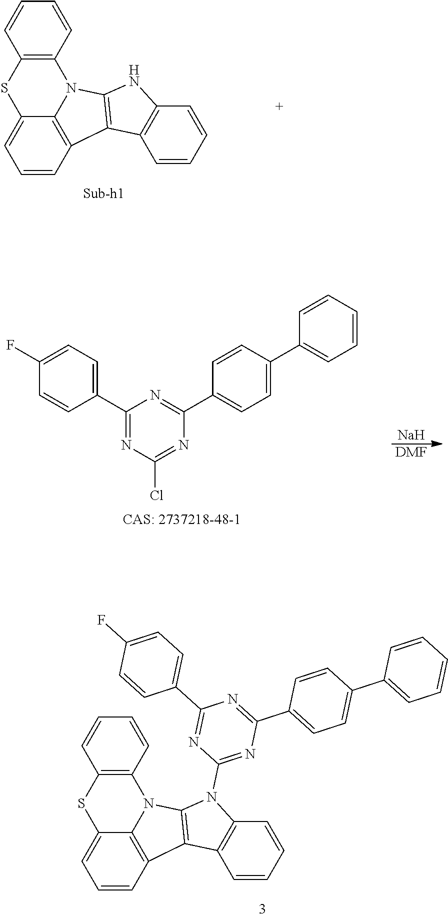

Reactant Sub-h1 (15.60 g, 50 mmol), reactant CAS: 2737218-48-1 (27.10 g, 75 mmol), and dried DMF (400 mL) were added sequentially to a 1000-mL three-necked flask. The reaction mixture was cooled to −10° C., followed by quickly adding sodium hydride (NaH, 60% w/w, 2.2 g, 55 mmol), and then stirred overnight. The reaction solution was poured into 500 mL of deionized water, stirred thoroughly for 30 minutes, and then filtered. The resulting solid was washed with deionized water to neutral, and then rinsed with absolute ethanol (200 mL), obtaining a crude product. The crude product was recrystallized with toluene, yielding green solid Compound 3 (24.85 g, yield 78%), m/z=638.2[M+H]⁺.

Compounds of the present disclosure shown in Table 10 were synthesized respectively by following the synthesis of Compound 3, with reactant CAS: 2737218-48-1 being replaced with a corresponding reactant N shown in Table 10.

TABLE 10
Synthesis of compounds of the present disclosure

| | Compound of the present | m/z | |
|---|---|---|---|
| Reactant N | disclosure | ([M + H]⁺) | Yield/% |
| 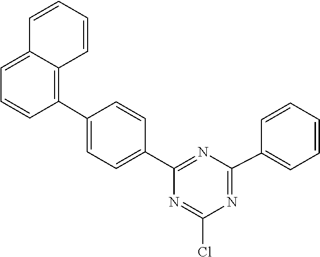 [SMILES: Clc1nc(-c2ccccc2)nc(-c2ccc(-c3cccc4ccccc34)cc2)n1] | 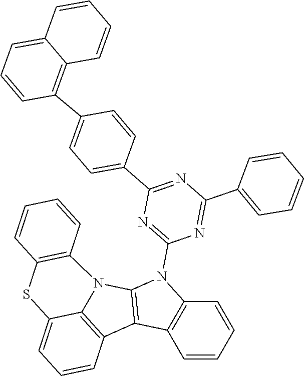 [SMILES: c1ccc(-c2nc(-c3ccc(-c4cccc5ccccc45)cc3)nc(-n3c4ccccc4c4c5cccc6c5n(c43)-c3ccccc3S6)n2)cc1] | 670.2 | 76 |
| CAS: 2229752-36-5 | 7 | | |
| 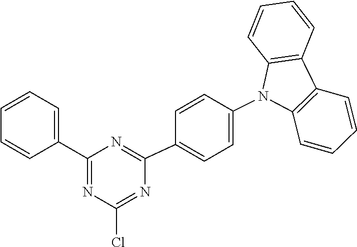 [SMILES: Clc1nc(-c2ccccc2)nc(-c2ccc(-n3c4ccccc4c4ccccc43)cc2)n1] | 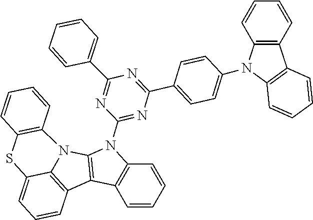 [SMILES: c1ccc(-c2nc(-c3ccc(-n4c5ccccc5c5ccccc54)cc3)nc(-n3c4ccccc4c4c5cccc6c5n(c43)-c3ccccc3S6)n2)cc1] | 709.2 | 71 |
| CAS: 1477759-28-6 | 10 | | |
| 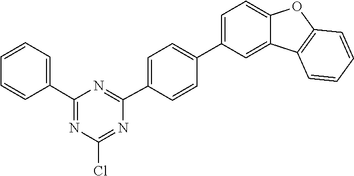 [SMILES: Clc1nc(-c2ccccc2)nc(-c2ccc(-c3ccc4oc5ccccc5c4c3)cc2)n1] | 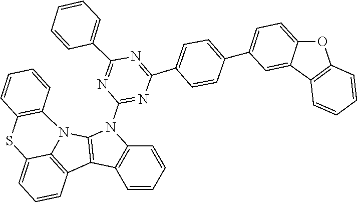 [SMILES: c1ccc(-c2nc(-c3ccc(-c4ccc5oc6ccccc6c5c4)cc3)nc(-n3c4ccccc4c4c5cccc6c5n(c43)-c3ccccc3S6)n2)cc1] | 710.2 | 75 |
| CAS: 2348384-37-0 | 12 | | |
| 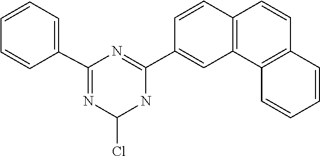 [SMILES: ClC1N=C(c2ccccc2)N=C(c2ccc3ccc4ccccc4c3c2)N1] | 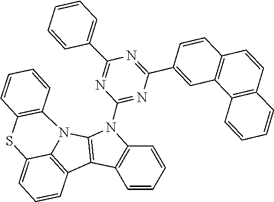 [SMILES: c1ccc(-c2nc(-c3ccc4ccc5ccccc5c4c3)nc(-n3c4ccccc4c4c5cccc6c5n(c43)-c3ccccc3S6)n2)cc1] | 644.2 | 80 |
| CAS: 1821147-84-5 | 22 | | |
| 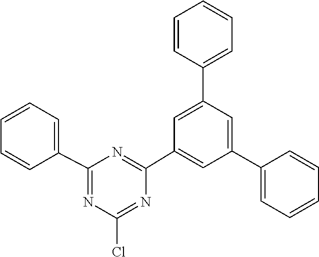 [SMILES: Clc1nc(-c2ccccc2)nc(-c2cc(-c3ccccc3)cc(-c3ccccc3)c2)n1] | 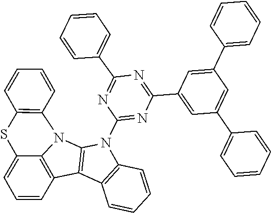 [SMILES: c1ccc(-c2cc(-c3ccccc3)cc(-c3nc(-c4ccccc4)nc(-n4c5ccccc5c5c6cccc7c6n(c54)-c4ccccc4S7)n3)c2)cc1] | 696.2 | 73 |
| CAS: 1616231-57-2 | 27 | | |
| 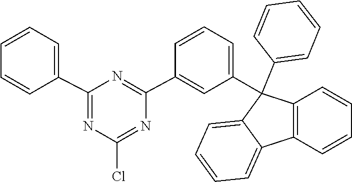 [SMILES: Clc1nc(-c2ccccc2)nc(-c2cccc(C3(c4ccccc4)c4ccccc4-c4ccccc43)c2)n1] | 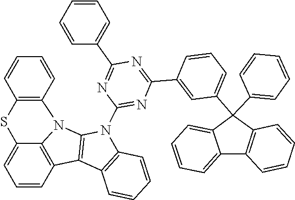 [SMILES: c1ccc(-c2nc(-c3cccc(C4(c5ccccc5)c5ccccc5-c5ccccc54)c3)nc(-n3c4ccccc4c4c5cccc6c5n(c43)-c3ccccc3S6)n2)cc1] | 784.3 | 60 |
| CAS: 1870820-52-2 | 29 | | |
| 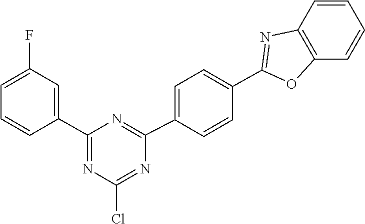 [SMILES: Fc1cccc(-c2nc(Cl)nc(-c3ccc(-c4nc5ccccc5o4)cc3)n2)c1] | 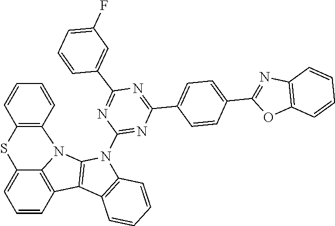 [SMILES: Fc1cccc(-c2nc(-c3ccc(-c4nc5ccccc5o4)cc3)nc(-n3c4ccccc4c4c5cccc6c5n(c43)-c3ccccc3S6)n2)c1] | 679.2 | 77 |
| Sub-j2 | 33 | | |
| 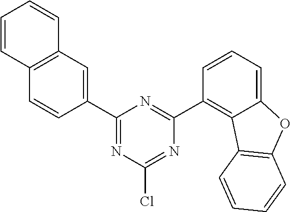 [SMILES: Clc1nc(-c2ccc3ccccc3c2)nc(-c2cccc3oc4ccccc4c23)n1] | 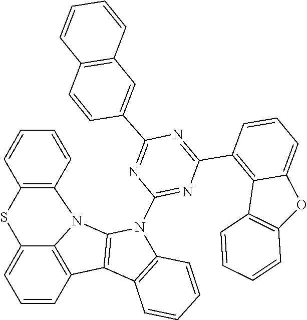 [SMILES: c1ccc2c(c1)Sc1cccc3c4c5ccccc5n(-c5nc(-c6ccc7ccccc7c6)nc(-c6cccc7oc8ccccc8c67)n5)c4n-2c13] | 684.2 | 79 |
| CAS: 2418528-30-8 | 42 | | |
| 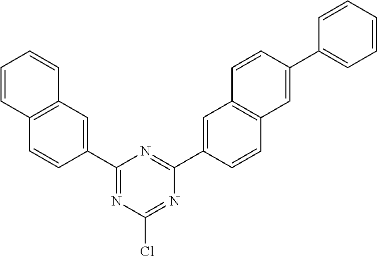 [SMILES: Clc1nc(-c2ccc3ccccc3c2)nc(-c2ccc3cc(-c4ccccc4)ccc3c2)n1] | 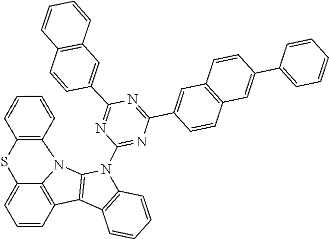 [SMILES: c1ccc(-c2ccc3cc(-c4nc(-c5ccc6ccccc6c5)nc(-n5c6ccccc6c6c7cccc8c7n(c65)-c5ccccc5S8)n4)ccc3c2)cc1] | 720.2 | 77 |
| CAS: 2636730-36-2 | 58 | | |
| 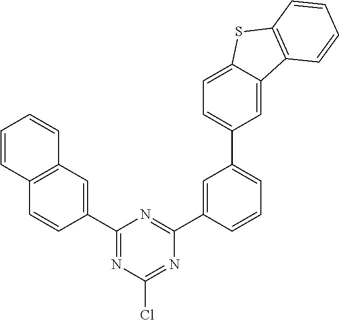 [SMILES: Clc1nc(-c2cccc(-c3ccc4sc5ccccc5c4c3)c2)nc(-c2ccc3ccccc3c2)n1] | 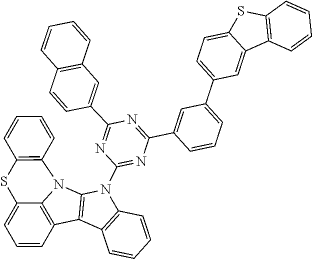 [SMILES: c1cc(-c2ccc3sc4ccccc4c3c2)cc(-c2nc(-c3ccc4ccccc4c3)nc(-n3c4ccccc4c4c5cccc6c5n(c43)-c3ccccc3S6)n2)c1] | 776.2 | 70 |
| Sub-j3 | 64 | | |
| 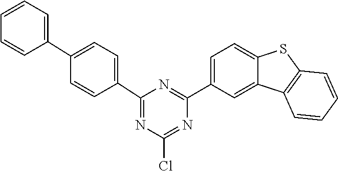 [SMILES: Clc1nc(-c2ccc(-c3ccccc3)cc2)nc(-c2ccc3sc4ccccc4c3c2)n1] | 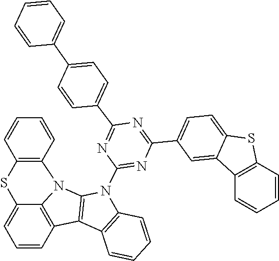 [SMILES: c1ccc(-c2ccc(-c3nc(-c4ccc5sc6ccccc6c5c4)nc(-n4c5ccccc5c5c6cccc7c6n(c54)-c4ccccc4S7)n3)cc2)cc1] | 726.2 | 71 |
| CAS: 2511641-36-2 | 70 | | |
| 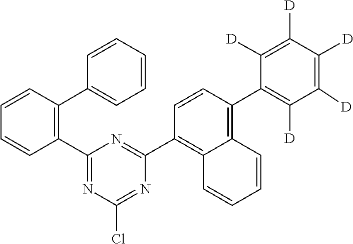 [SMILES: [2H]c1c([2H])c([2H])c(-c2ccc(-c3nc(Cl)nc(-c4ccccc4-c4ccccc4)n3)c3ccccc23)c([2H])c1[2H]] | 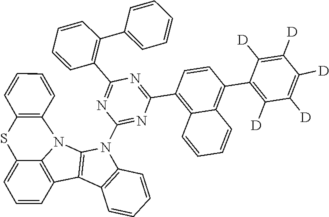 [SMILES: [2H]c1c([2H])c([2H])c(-c2ccc(-c3nc(-c4ccccc4-c4ccccc4)nc(-n4c5ccccc5c5c6cccc7c6n(c54)-c4ccccc4S7)n3)c3ccccc23)c([2H])c1[2H]] | 751.3 | 72 |
| Sub-j4 | 75 | | |

### Synthesis of Compound 80

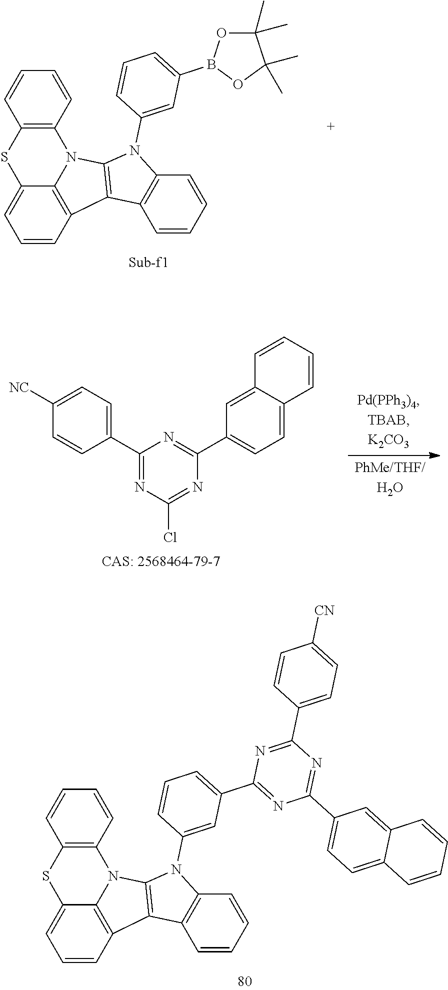 [SMILES: CC1(C)OB(c2cccc(-n3c4ccccc4c4c5cccc6c5n(c43)-c3ccccc3S6)c2)OC1(C)C ; N#Cc1ccc(-c2nc(Cl)nc(-c3ccc4ccccc4c3)n2)cc1 ; N#Cc1ccc(-c2nc(-c3cccc(-n4c5ccccc5c5c6cccc7c6n(c54)-c4ccccc4S7)c3)nc(-c3ccc4ccccc4c3)n2)cc1]

Reactant Sub-f1 (18.51 g, 36 mmol), reactant CAS: 2568464-79-7 (10.26 g, 30 mmol), tetrakis(triphenylphosphine)palladium (Pd(PPh₃)₄, 0.42 g, 0.36 mmol), tetrabutylammonium bromide (TBAB, 1.16 g, 3.6 mmol), anhydrous potassium carbonate (K₂CO₃, 9.95 g, 72 mmol), toluene (PhMe, 180 mL), tetrahydrofuran (THF, 45 mL), and deionized water (H₂O, 45 mL) was added sequentially to a 500-mL three-necked flask under a nitrogen atmosphere, heated to reflux and stirred for 12 hours. After being cooled to room temperature, the reaction solution was extracted with dichloromethane (100 mL×3 times). The resulting organic phase was dried over anhydrous magnesium sulfate, filtered and distilled under reduced pressure to remove the solvent, obtaining a crude product. The crude product was purified by silica gel column chromatography with n-heptane/dichloromethane as a mobile phase, yielding white solid Compound 80 (15.60 g, yield 75%), m/z=695.2 [M+H]⁺.

The following compounds of the present disclosure were synthesized respectively by following the synthesis of Compound 80, with reactant Sub-f1 being replaced with a corresponding reactant O shown in Table 11, and with reactant CAS: 2568464-79-7 being replaced with a corresponding reactant P.

TABLE 11
Synthesis of compounds of the present disclosure

| | | Compound of the present | m/z | |
|---|---|---|---|---|
| Reactant O | Reactant P | disclosure | ([M + H]⁺) | Yield/% |
| 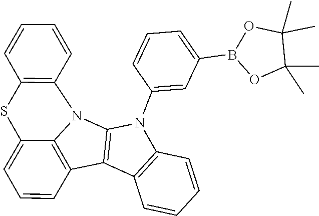 [SMILES: CC1(C)OB(c2cccc(-n3c4ccccc4c4c5cccc6c5n(c43)-c3ccccc3S6)c2)OC1(C)C] | 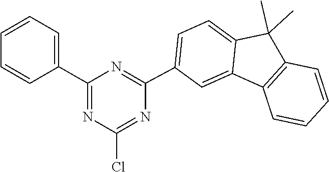 [SMILES: CC1(C)c2ccccc2-c2cc(-c3nc(Cl)nc(-c4ccccc4)n3)ccc21] | 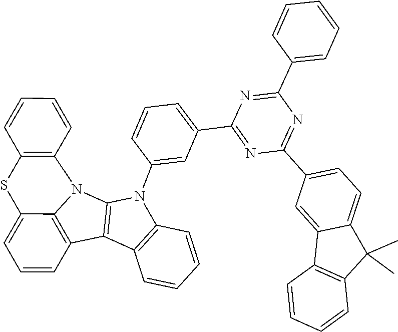 [SMILES: CC1(C)c2ccccc2-c2cc(-c3nc(-c4ccccc4)nc(-c4cccc(-n5c6ccccc6c6c7cccc8c7n(c65)-c5ccccc5S8)c4)n3)ccc21] | 736.3 | 73 |
| Sub-f1 | CAS: 1835683-72-1 | 100 | | |
| 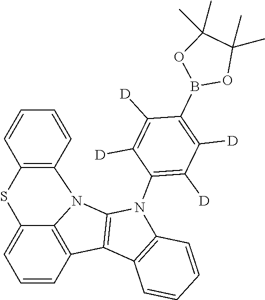 [SMILES: [2H]c1c([2H])c(-n2c3ccccc3c3c4cccc5c4n(c32)-c2ccccc2S5)c([2H])c([2H])c1B1OC(C)(C)C(C)(C)O1] | 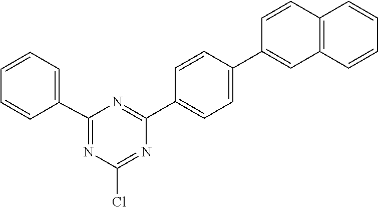 [SMILES: Clc1nc(-c2ccccc2)nc(-c2ccc(-c3ccc4ccccc4c3)cc2)n1] | 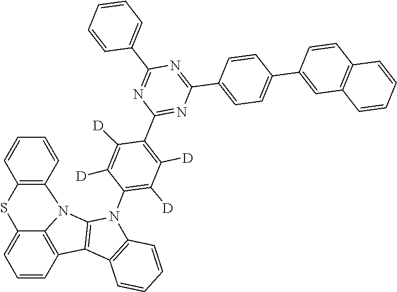 [SMILES: [2H]c1c([2H])c(-n2c3ccccc3c3c4cccc5c4n(c32)-c2ccccc2S5)c([2H])c([2H])c1-c1nc(-c2ccccc2)nc(-c2ccc(-c3ccc4ccccc4c3)cc2)n1] | 750.3 | 73 |
| Sub-f6 | CAS: 2396648-13-6 | 101 | | |
| 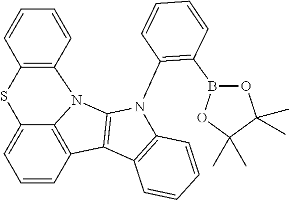 [SMILES: CC1(C)OB(c2ccccc2-n2c3ccccc3c3c4cccc5c4n(c32)-c2ccccc2S5)OC1(C)C] | 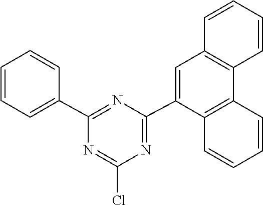 [SMILES: Clc1nc(-c2ccccc2)nc(-c2cc3ccccc3c3ccccc23)n1] | 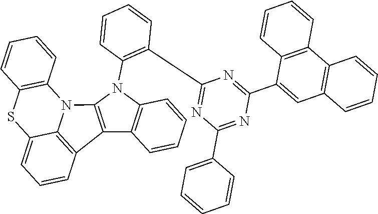 [SMILES: c1ccc(-c2nc(-c3ccccc3-n3c4ccccc4c4c5cccc6c5n(c43)-c3ccccc3S6)nc(-c3cc4ccccc4c4ccccc34)n2)cc1] | 720.2 | 58 |
| Sub-f7 | CAS: 1821147-80-1 | 120 | | |
| 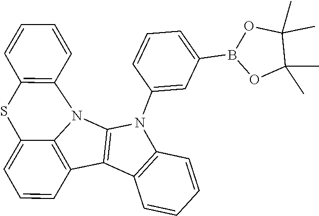 [SMILES: CC1(C)OB(c2cccc(-n3c4ccccc4c4c5cccc6c5n(c43)-c3ccccc3S6)c2)OC1(C)C] | 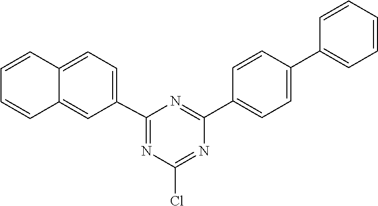 [SMILES: Clc1nc(-c2ccc(-c3ccccc3)cc2)nc(-c2ccc3ccccc3c2)n1] | 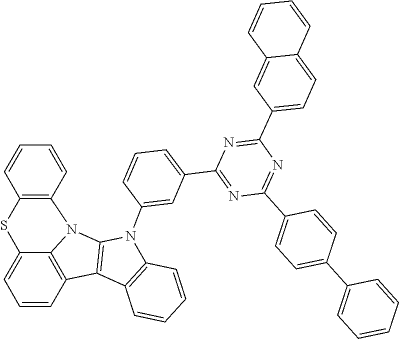 [SMILES: c1ccc(-c2ccc(-c3nc(-c4cccc(-n5c6ccccc6c6c7cccc8c7n(c65)-c5ccccc5S8)c4)nc(-c4ccc5ccccc5c4)n3)cc2)cc1] | 746.2 | 67 |
| Sub-f1 | CAS: 2307664-39-5 | 132 | | |
| 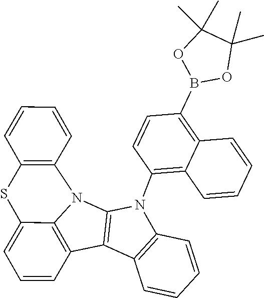 [SMILES: CC1(C)OB(c2ccc(-n3c4ccccc4c4c5cccc6c5n(c43)-c3ccccc3S6)c3ccccc23)OC1(C)C] | 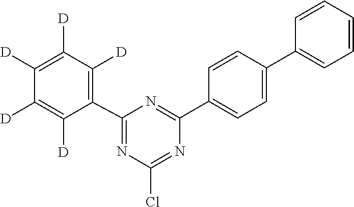 [SMILES: [2H]c1c([2H])c([2H])c(-c2nc(Cl)nc(-c3ccc(-c4ccccc4)cc3)n2)c([2H])c1[2H]] | 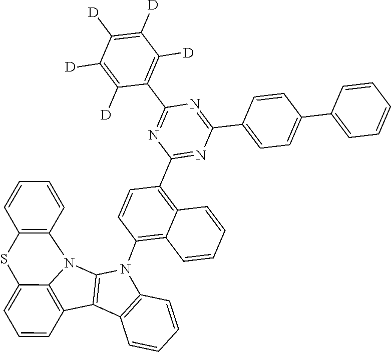 [SMILES: [2H]c1c([2H])c([2H])c(-c2nc(-c3ccc(-c4ccccc4)cc3)nc(-c3ccc(-n4c5ccccc5c5c6cccc7c6n(c54)-c4ccccc4S7)c4ccccc34)n2)c([2H])c1[2H]] | 751.3 | 70 |
| Sub-f8 | CAS: 2401923-63-3 | 187 | | |
| 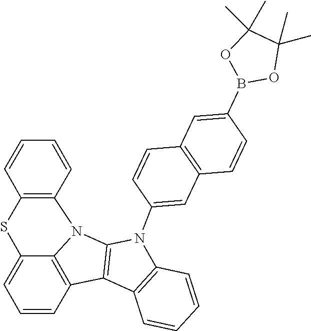 [SMILES: CC1(C)OB(c2ccc3cc(-n4c5ccccc5c5c6cccc7c6n(c54)-c4ccccc4S7)ccc3c2)OC1(C)C] | 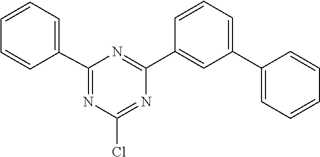 [SMILES: Clc1nc(-c2ccccc2)nc(-c2cccc(-c3ccccc3)c2)n1] | 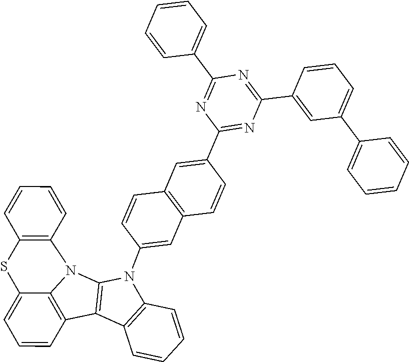 [SMILES: c1ccc(-c2cccc(-c3nc(-c4ccccc4)nc(-c4ccc5cc(-n6c7ccccc7c7c8cccc9c8n(c76)-c6ccccc6S9)ccc5c4)n3)c2)cc1] | 746.2 | 66 |
| Sub-f9 | CAS: 1689576-03-1 | 178 | | |
| 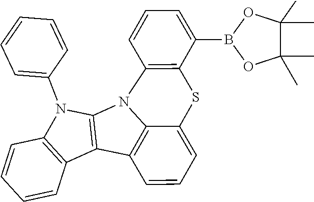 [SMILES: CC1(C)OB(c2cccc3c2Sc2cccc4c5c6ccccc6n(-c6ccccc6)c5n-3c24)OC1(C)C] | 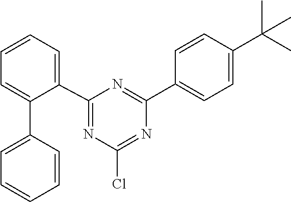 [SMILES: CC(C)(C)c1ccc(-c2nc(Cl)nc(-c3ccccc3-c3ccccc3)n2)cc1] | 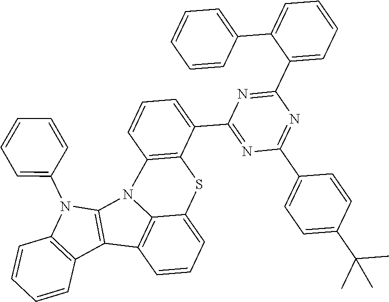 [SMILES: CC(C)(C)c1ccc(-c2nc(-c3ccccc3-c3ccccc3)nc(-c3cccc4c3Sc3cccc5c6c7ccccc7n(-c7ccccc7)c6n-4c35)n2)cc1] | 752.3 | 68 |
| Sub-f4 | Sub-j5 | 201 | | |
| 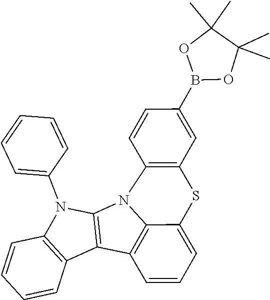 [SMILES: CC1(C)OB(c2ccc3c(c2)Sc2cccc4c5c6ccccc6n(-c6ccccc6)c5n-3c24)OC1(C)C] | 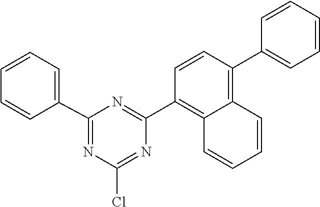 [SMILES: Clc1nc(-c2ccccc2)nc(-c2ccc(-c3ccccc3)c3ccccc23)n1] | 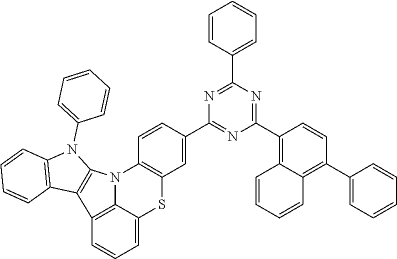 [SMILES: c1ccc(-c2nc(-c3ccc4c(c3)Sc3cccc5c6c7ccccc7n(-c7ccccc7)c6n-4c35)nc(-c3ccc(-c4ccccc4)c4ccccc34)n2)cc1] | 746.2 | 72 |
| Sub-f3 | CAS: 2418702-67-5 | 209 | | |
| 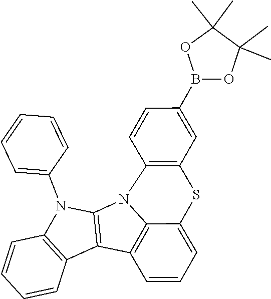 [SMILES: CC1(C)OB(c2ccc3c(c2)Sc2cccc4c5c6ccccc6n(-c6ccccc6)c5n-3c24)OC1(C)C] | 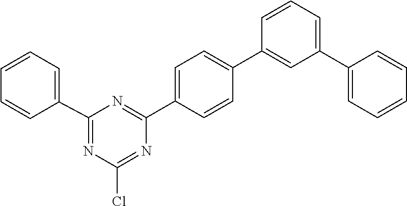 [SMILES: Clc1nc(-c2ccccc2)nc(-c2ccc(-c3cccc(-c4ccccc4)c3)cc2)n1] | 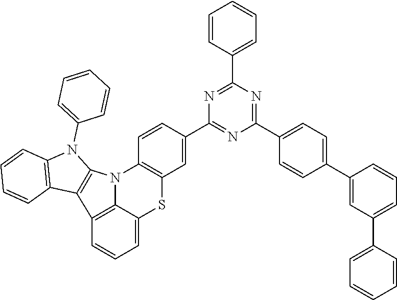 [SMILES: c1ccc(-c2cccc(-c3ccc(-c4nc(-c5ccccc5)nc(-c5ccc6c(c5)Sc5cccc7c8c9ccccc9n(-c9ccccc9)c8n-6c57)n4)cc3)c2)cc1] | 772.3 | 72 |
| Sub-f3 | CAS: 2173555-95-6 | 212 | | |
| 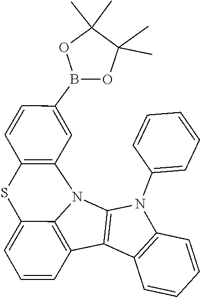 [SMILES: CC1(C)OB(c2ccc3c(c2)-n2c4c(cccc4c4c5ccccc5n(-c5ccccc5)c42)S3)OC1(C)C] | 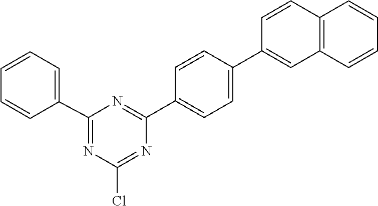 [SMILES: Clc1nc(-c2ccccc2)nc(-c2ccc(-c3ccc4ccccc4c3)cc2)n1] | 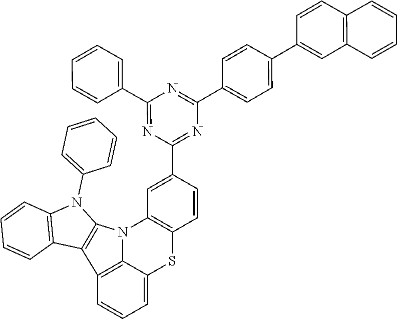 [SMILES: c1ccc(-c2nc(-c3ccc(-c4ccc5ccccc5c4)cc3)nc(-c3ccc4c(c3)-n3c5c(cccc5c5c6ccccc6n(-c6ccccc6)c53)S4)n2)cc1] | 746.2 | 72 |
| Sub-f2 | CAS: 2396648-13-6 | 229 | | |
| 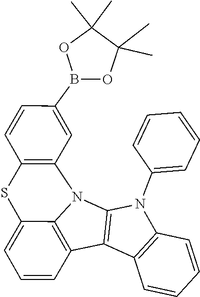 [SMILES: CC1(C)OB(c2ccc3c(c2)-n2c4c(cccc4c4c5ccccc5n(-c5ccccc5)c42)S3)OC1(C)C] | 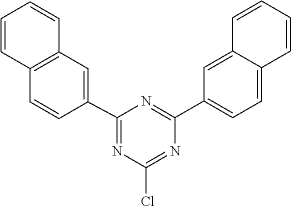 [SMILES: Clc1nc(-c2ccc3ccccc3c2)nc(-c2ccc3ccccc3c2)n1] | 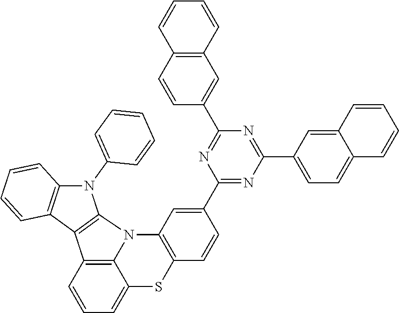 [SMILES: c1ccc(-n2c3ccccc3c3c4cccc5c4n(c32)-c2cc(-c3nc(-c4ccc6ccccc6c4)nc(-c4ccc6ccccc6c4)n3)ccc2S5)cc1] | 720.2 | 66 |
| Sub-f2 | CAS: 1247124-77-1 | 235 | | |
| 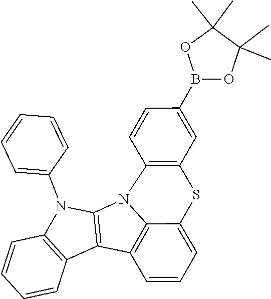 [SMILES: CC1(C)OB(c2ccc3c(c2)Sc2cccc4c5c6ccccc6n(-c6ccccc6)c5n-3c24)OC1(C)C] | 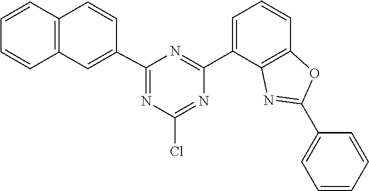 [SMILES: Clc1nc(-c2ccc3ccccc3c2)nc(-c2cccc3oc(-c4ccccc4)nc23)n1] | 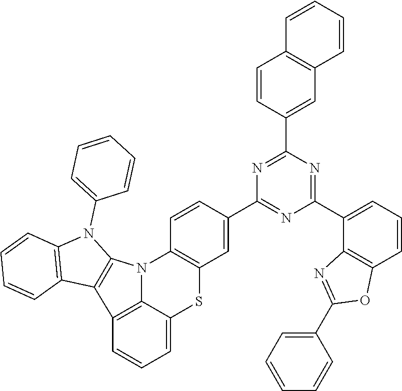 [SMILES: c1ccc(-c2nc3c(-c4nc(-c5ccc6c(c5)Sc5cccc7c8c9ccccc9n(-c9ccccc9)c8n-6c57)nc(-c5ccc6ccccc6c5)n4)cccc3o2)cc1] | 787.2 | 75 |
| Sub-f3 | Sub-j6 | 263 | | |
| 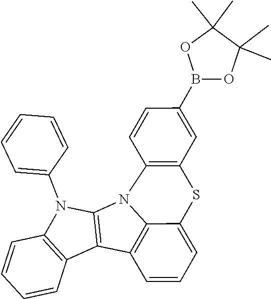 [SMILES: CC1(C)OB(c2ccc3c(c2)Sc2cccc4c5c6ccccc6n(-c6ccccc6)c5n-3c24)OC1(C)C] | 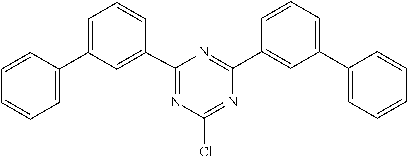 [SMILES: Clc1nc(-c2cccc(-c3ccccc3)c2)nc(-c2cccc(-c3ccccc3)c2)n1] | 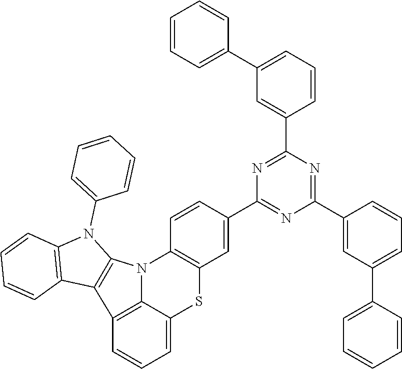 [SMILES: c1ccc(-c2cccc(-c3nc(-c4cccc(-c5ccccc5)c4)nc(-c4ccc5c(c4)Sc4cccc6c7c8ccccc8n(-c8ccccc8)c7n-5c46)n3)c2)cc1] | 772.3 | 72 |
| Sub-f3 | CAS: 1205748-61-3 | 272 | | |
| 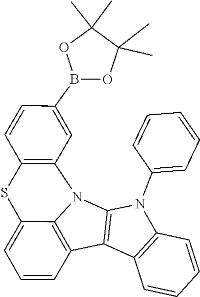 [SMILES: CC1(C)OB(c2ccc3c(c2)-n2c4c(cccc4c4c5ccccc5n(-c5ccccc5)c42)S3)OC1(C)C] | 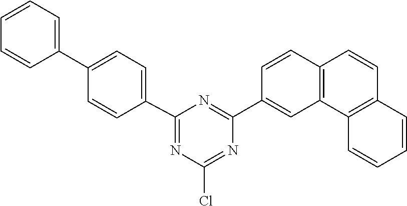 [SMILES: Clc1nc(-c2ccc(-c3ccccc3)cc2)nc(-c2ccc3ccc4ccccc4c3c2)n1] | 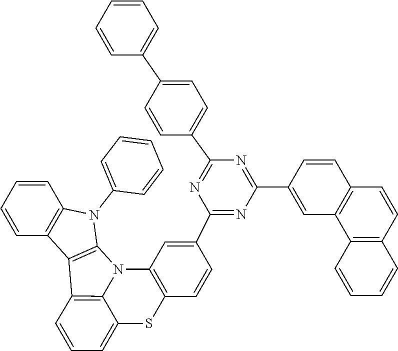 [SMILES: c1ccc(-c2ccc(-c3nc(-c4ccc5c(c4)-n4c6c(cccc6c6c7ccccc7n(-c7ccccc7)c64)S5)nc(-c4ccc5ccc6ccccc6c5c4)n3)cc2)cc1] | 796.3 | 68 |
| Sub-f2 | Sub-j1 | 283 | | |
| 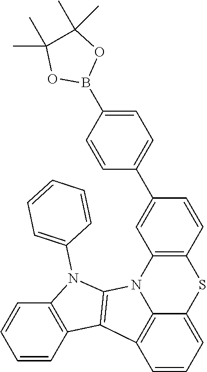 [SMILES: CC1(C)OB(c2ccc(-c3ccc4c(c3)-n3c5c(cccc5c5c6ccccc6n(-c6ccccc6)c53)S4)cc2)OC1(C)C] | 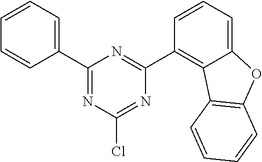 [SMILES: Clc1nc(-c2ccccc2)nc(-c2cccc3oc4ccccc4c23)n1] | 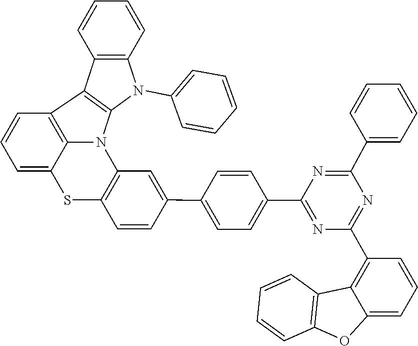 [SMILES: c1ccc(-c2nc(-c3ccc(-c4ccc5c(c4)-n4c6c(cccc6c6c7ccccc7n(-c7ccccc7)c64)S5)cc3)nc(-c3cccc4oc5ccccc5c34)n2)cc1] | 786.2 | 69 |
| Sub-f12 | CAS: 1883265-32-4 | 292 | | |
| 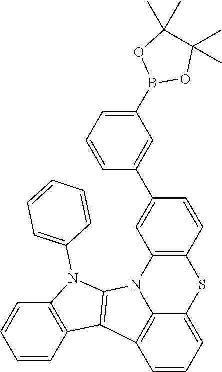 [SMILES: CC1(C)OB(c2cccc(-c3ccc4c(c3)-n3c5c(cccc5c5c6ccccc6n(-c6ccccc6)c53)S4)c2)OC1(C)C] | 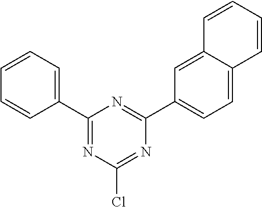 [SMILES: Clc1nc(-c2ccccc2)nc(-c2ccc3ccccc3c2)n1] | 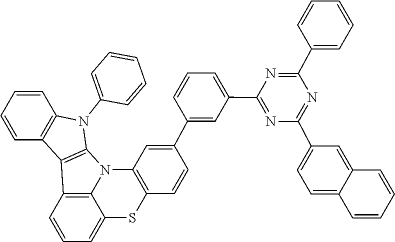 [SMILES: c1ccc(-c2nc(-c3cccc(-c4ccc5c(c4)-n4c6c(cccc6c6c7ccccc7n(-c7ccccc7)c64)S5)c3)nc(-c3ccc4ccccc4c3)n2)cc1] | 746.2 | 73 |
| Sub-f14 | CAS: 1342819-12-8 | 301 | | |
| 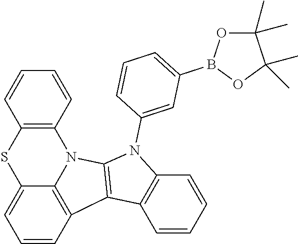 [SMILES: CC1(C)OB(c2cccc(-n3c4ccccc4c4c5cccc6c5n(c43)-c3ccccc3S6)c2)OC1(C)C] | 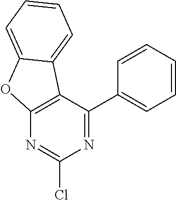 [SMILES: Clc1nc(-c2ccccc2)c2c(n1)oc1ccccc12] | 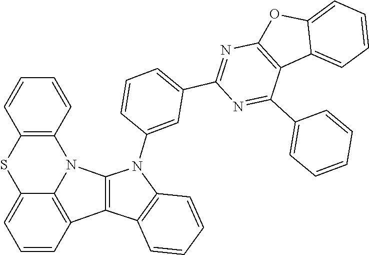 [SMILES: c1ccc(-c2nc(-c3cccc(-n4c5ccccc5c5c6cccc7c6n(c54)-c4ccccc4S7)c3)nc3oc4ccccc4c23)cc1] | 633.2 | 74 |
| Sub-f1 | CAS: 1801325-93-8 | 377 | | |
| 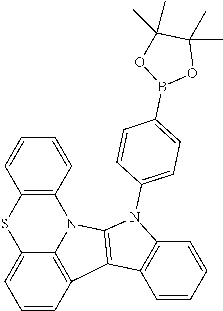 [SMILES: CC1(C)OB(c2ccc(-n3c4ccccc4c4c5cccc6c5n(c43)-c3ccccc3S6)cc2)OC1(C)C] | 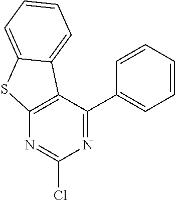 [SMILES: Clc1nc(-c2ccccc2)c2c(n1)sc1ccccc12] | 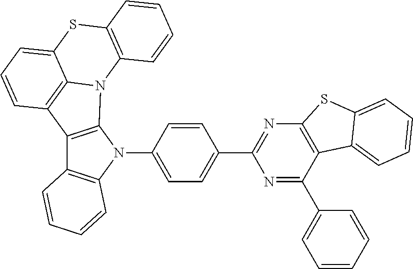 [SMILES: c1ccc(-c2nc(-c3ccc(-n4c5ccccc5c5c6cccc7c6n(c54)-c4ccccc4S7)cc3)nc3sc4ccccc4c23)cc1] | 649.2 | 66 |
| Sub-f5 | CAS: 1801325-97-2 | 382 | | |
| 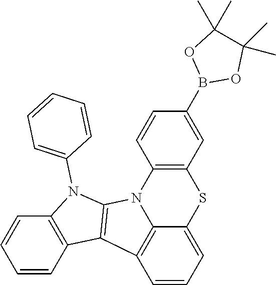 [SMILES: CC1(C)OB(c2ccc3c(c2)Sc2cccc4c5c6ccccc6n(-c6ccccc6)c5n-3c24)OC1(C)C] | 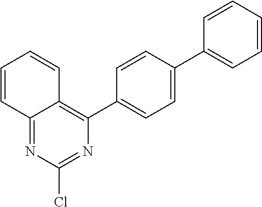 [SMILES: Clc1nc(-c2ccc(-c3ccccc3)cc2)c2ccccc2n1] | 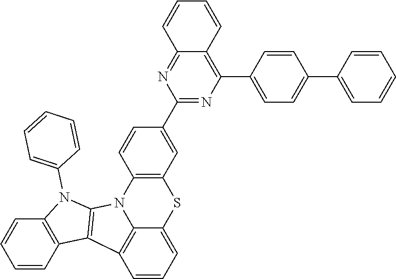 [SMILES: c1ccc(-c2ccc(-c3nc(-c4ccc5c(c4)Sc4cccc6c7c8ccccc8n(-c8ccccc8)c7n-5c46)nc4ccccc34)cc2)cc1] | 669.2 | 67 |
| Sub-f3 | CAS: 1262866-93-2 | 415 | | |
| 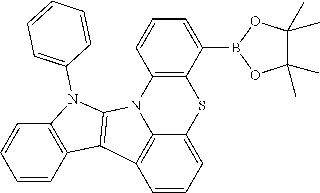 [SMILES: CC1(C)OB(c2cccc3c2Sc2cccc4c5c6ccccc6n(-c6ccccc6)c5n-3c24)OC1(C)C] | 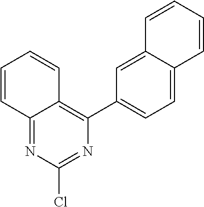 [SMILES: Clc1nc(-c2ccc3ccccc3c2)c2ccccc2n1] | 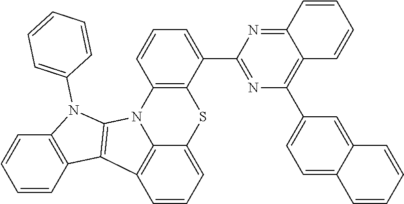 [SMILES: c1ccc(-n2c3ccccc3c3c4cccc5c4n(c32)-c2cccc(-c3nc(-c4ccc6ccccc6c4)c4ccccc4n3)c2S5)cc1] | 643.2 | 65 |
| Sub-f4 | CAS: 124959-44-0 | 421 | | |
| 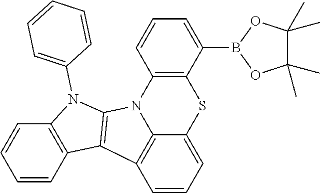 [SMILES: CC1(C)OB(c2cccc3c2Sc2cccc4c5c6ccccc6n(-c6ccccc6)c5n-3c24)OC1(C)C] | 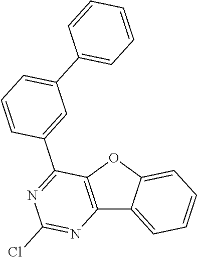 [SMILES: Clc1nc(-c2cccc(-c3ccccc3)c2)c2oc3ccccc3c2n1] | 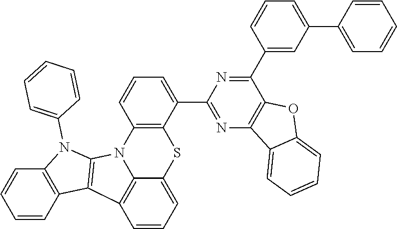 [SMILES: c1ccc(-c2cccc(-c3nc(-c4cccc5c4Sc4cccc6c7c8ccccc8n(-c8ccccc8)c7n-5c46)nc4c3oc3ccccc34)c2)cc1] | 709.2 | 70 |
| Sub-f4 | CAS: 1801420-89-2 | 424 | | |
| 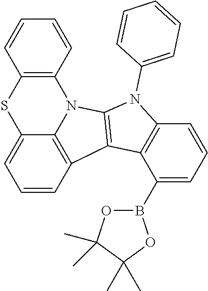 [SMILES: CC1(C)OB(c2cccc3c2c2c4cccc5c4n(c2n3-c2ccccc2)-c2ccccc2S5)OC1(C)C] | 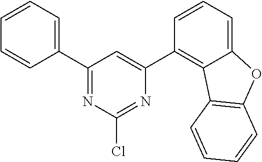 [SMILES: Clc1nc(-c2ccccc2)cc(-c2cccc3oc4ccccc4c23)n1] | 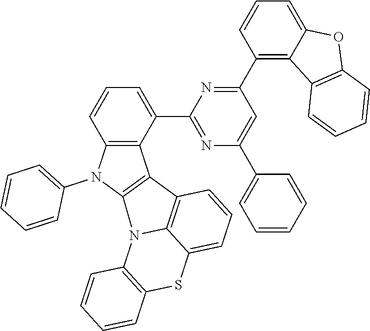 [SMILES: c1ccc(-c2cc(-c3cccc4oc5ccccc5c34)nc(-c3cccc4c3c3c5cccc6c5n(c3n4-c3ccccc3)-c3ccccc3S6)n2)cc1] | 709.2 | 66 |
| Sub-f13 | CAS: 1883265-43-7 | 468 | | |
| 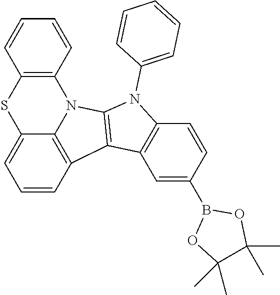 [SMILES: CC1(C)OB(c2ccc3c(c2)c2c4cccc5c4n(c2n3-c2ccccc2)-c2ccccc2S5)OC1(C)C] | 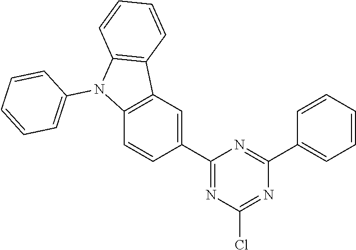 [SMILES: Clc1nc(-c2ccccc2)nc(-c2ccc3c(c2)c2ccccc2n3-c2ccccc2)n1] | 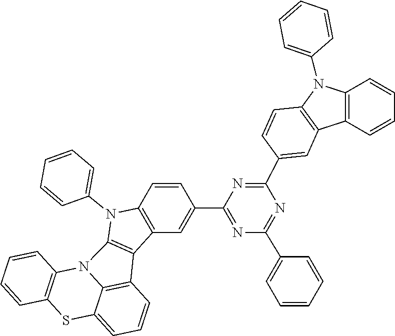 [SMILES: c1ccc(-c2nc(-c3ccc4c(c3)c3ccccc3n4-c3ccccc3)nc(-c3ccc4c(c3)c3c5cccc6c5n(c3n4-c3ccccc3)-c3ccccc3S6)n2)cc1] | 785.3 | 65 |
| Sub-f11 | CAS: 1476785-42-8 | 473 | | |
| 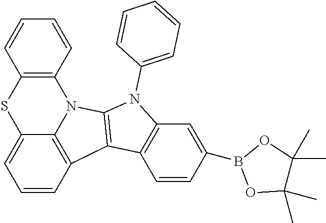 [SMILES: CC1(C)OB(c2ccc3c4c5cccc6c5n(c4n(-c4ccccc4)c3c2)-c2ccccc2S6)OC1(C)C] | 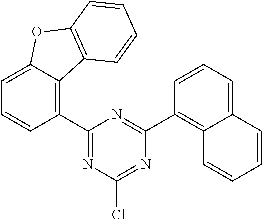 [SMILES: Clc1nc(-c2cccc3ccccc23)nc(-c2cccc3oc4ccccc4c23)n1] | 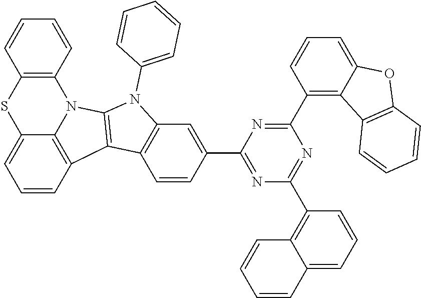 [SMILES: c1ccc(-n2c3cc(-c4nc(-c5cccc6ccccc56)nc(-c5cccc6oc7ccccc7c56)n4)ccc3c3c4cccc5c4n(c32)-c2ccccc2S5)cc1] | 760.2 | 73 |
| Sub-f10 | CAS: 2417986-80-0 | 499 | | |
| 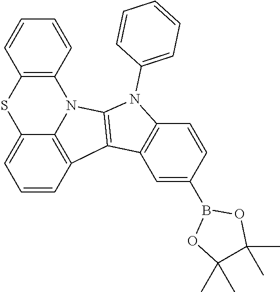 [SMILES: CC1(C)OB(c2ccc3c(c2)c2c4cccc5c4n(c2n3-c2ccccc2)-c2ccccc2S5)OC1(C)C] | 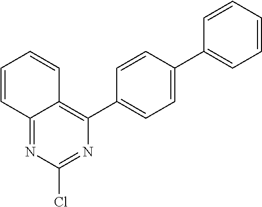 [SMILES: Clc1nc(-c2ccc(-c3ccccc3)cc2)c2ccccc2n1] | 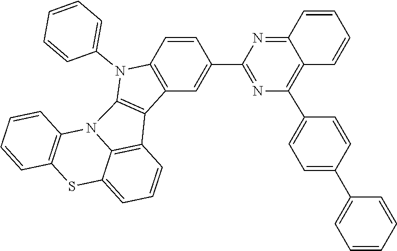 [SMILES: c1ccc(-c2ccc(-c3nc(-c4ccc5c(c4)c4c6cccc7c6n(c4n5-c4ccccc4)-c4ccccc4S7)nc4ccccc34)cc2)cc1] | 669.2 | 65 |
| Sub-f11 | CAS: 1262866-93-2 | 583 | | |
| 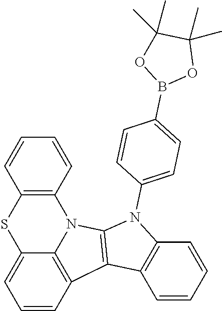 [SMILES: CC1(C)OB(c2ccc(-n3c4ccccc4c4c5cccc6c5n(c43)-c3ccccc3S6)cc2)OC1(C)C] | 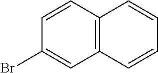 [SMILES: Brc1ccc2ccccc2c1] | 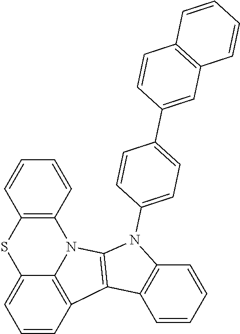 [SMILES: c1ccc2c(c1)Sc1cccc3c4c5ccccc5n(-c5ccc(-c6ccc7ccccc7c6)cc5)c4n-2c13] | 515.2 | 79 |
| Sub-f5 | CAS: 580-13-2 | 650 | | |
| 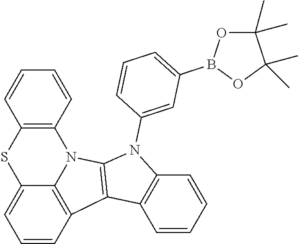 [SMILES: CC1(C)OB(c2cccc(-n3c4ccccc4c4c5cccc6c5n(c43)-c3ccccc3S6)c2)OC1(C)C] | 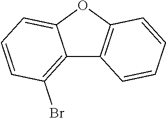 [SMILES: Brc1cccc2oc3ccccc3c12] | 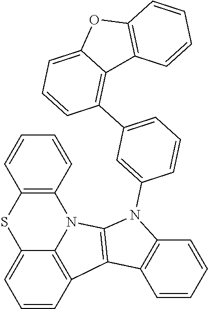 [SMILES: c1cc(-c2cccc3oc4ccccc4c23)cc(-n2c3ccccc3c3c4cccc5c4n(c32)-c2ccccc2S5)c1] | 555.2 | 77 |
| Sub-f1 | CAS: 50548-45-3 | 668 | | |
| 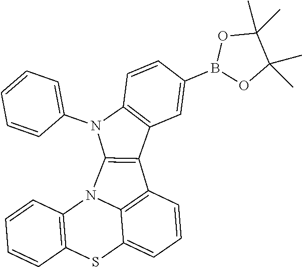 [SMILES: CC1(C)OB(c2ccc3c(c2)c2c4cccc5c4n(c2n3-c2ccccc2)-c2ccccc2S5)OC1(C)C] | 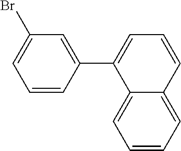 [SMILES: Brc1cccc(-c2cccc3ccccc23)c1] | 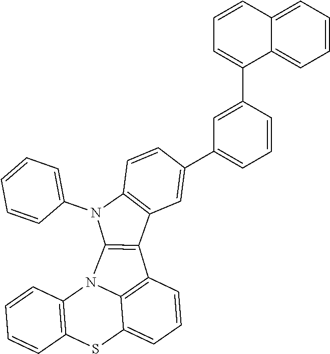 [SMILES: c1ccc(-n2c3ccc(-c4cccc(-c5cccc6ccccc56)c4)cc3c3c4cccc5c4n(c32)-c2ccccc2S5)cc1] | 591.2 | 72 |
| Sub-f11 | CAS: 853945-53-6 | 709 | | |
| 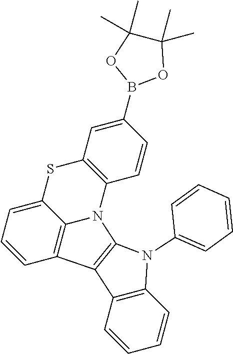 [SMILES: CC1(C)OB(c2ccc3c(c2)Sc2cccc4c5c6ccccc6n(-c6ccccc6)c5n-3c24)OC1(C)C] | 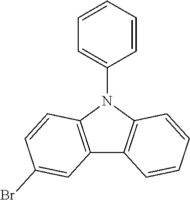 [SMILES: Brc1ccc2c(c1)c1ccccc1n2-c1ccccc1] | 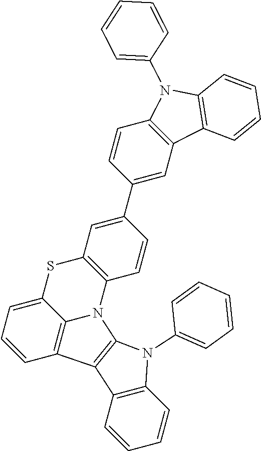 [SMILES: c1ccc(-n2c3ccccc3c3cc(-c4ccc5c(c4)Sc4cccc6c7c8ccccc8n(-c8ccccc8)c7n-5c46)ccc32)cc1] | 630.2 | 78 |
| Sub-f3 | CAS: 1153-85-1 | 716 | | |
| 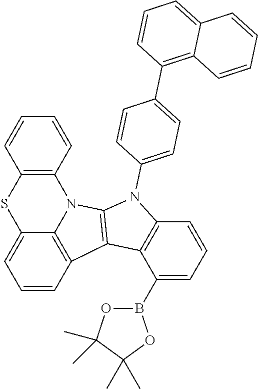 [SMILES: CC1(C)OB(c2cccc3c2c2c4cccc5c4n(c2n3-c2ccc(-c3cccc4ccccc34)cc2)-c2ccccc2S5)OC1(C)C] | 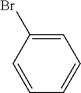 | 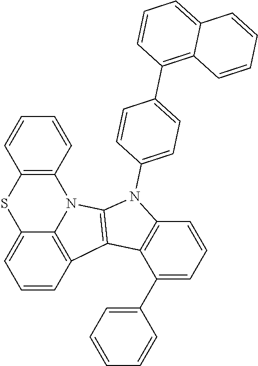 [SMILES: c1ccc(-c2cccc3c2c2c4cccc5c4n(c2n3-c2ccc(-c3cccc4ccccc34)cc2)-c2ccccc2S5)cc1] | 591.2 | 81 |
| Sub-h16 | CAS: 108-86-1 | 730 | | |
| 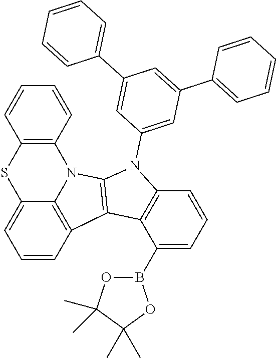 [SMILES: CC1(C)OB(c2cccc3c2c2c4cccc5c4n(c2n3-c2cc(-c3ccccc3)cc(-c3ccccc3)c2)-c2ccccc2S5)OC1(C)C] | 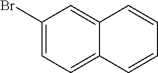 [SMILES: Brc1ccc2ccccc2c1] | 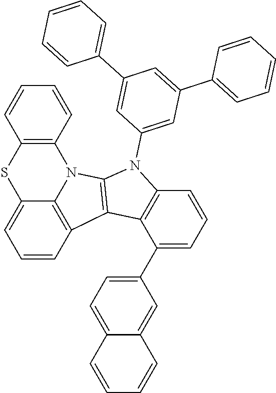 [SMILES: c1ccc(-c2cc(-c3ccccc3)cc(-n3c4cccc(-c5ccc6ccccc6c5)c4c4c5cccc6c5n(c43)-c3ccccc3S6)c2)cc1] | 667.2 | 78 |
| Sub-h15 | CAS: 580-13-2 | 758 | | |
| 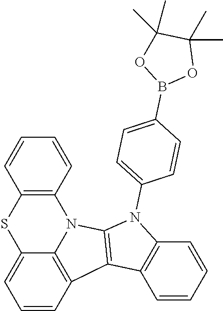 [SMILES: CC1(C)OB(c2ccc(-n3c4ccccc4c4c5cccc6c5n(c43)-c3ccccc3S6)cc2)OC1(C)C] | 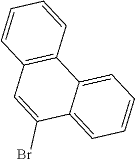 [SMILES: Brc1cc2ccccc2c2ccccc12] | 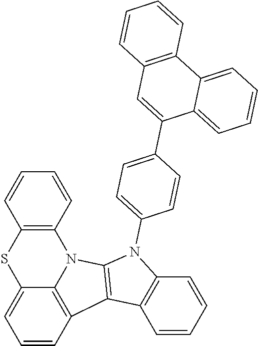 [SMILES: c1ccc2c(c1)Sc1cccc3c4c5ccccc5n(-c5ccc(-c6cc7ccccc7c7ccccc67)cc5)c4n-2c13] | 565.2 | 85 |
| Sub-h5 | CAS: 573-17-1 | 695 | | |
| 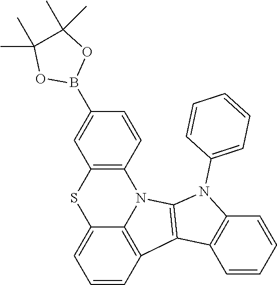 [SMILES: CC1(C)OB(c2ccc3c(c2)Sc2cccc4c5c6ccccc6n(-c6ccccc6)c5n-3c24)OC1(C)C] | 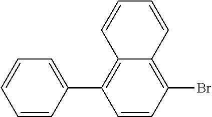 [SMILES: Brc1ccc(-c2ccccc2)c2ccccc12] | 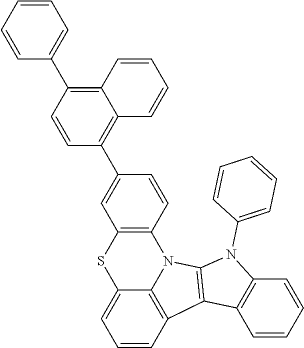 [SMILES: c1ccc(-c2ccc(-c3ccc4c(c3)Sc3cccc5c6c7ccccc7n(-c7ccccc7)c6n-4c35)c3ccccc23)cc1] | 591.2 | 79 |
| Sub-h3 | CAS: 59951-65-4 | 707 | | |
| 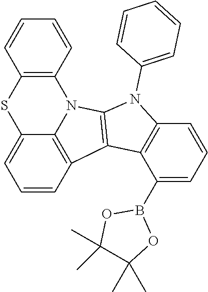 [SMILES: CC1(C)OB(c2cccc3c2c2c4cccc5c4n(c2n3-c2ccccc2)-c2ccccc2S5)OC1(C)C] | 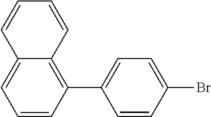 [SMILES: Brc1ccc(-c2cccc3ccccc23)cc1] | 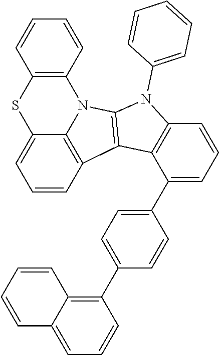 [SMILES: c1ccc(-n2c3cccc(-c4ccc(-c5cccc6ccccc56)cc4)c3c3c4cccc5c4n(c32)-c2ccccc2S5)cc1] | 591.2 | 73 |
| Sub-h12 | CAS: 204530-94-9 | 705 | | |
| 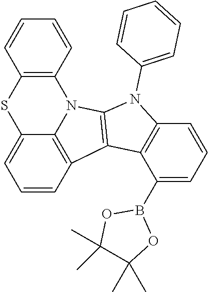 [SMILES: CC1(C)OB(c2cccc3c2c2c4cccc5c4n(c2n3-c2ccccc2)-c2ccccc2S5)OC1(C)C] | 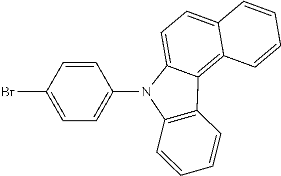 [SMILES: Brc1ccc(-n2c3ccccc3c3c4ccccc4ccc32)cc1] | 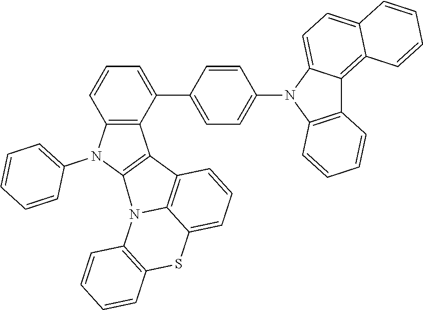 [SMILES: c1ccc(-n2c3cccc(-c4ccc(-n5c6ccccc6c6c7ccccc7ccc65)cc4)c3c3c4cccc5c4n(c32)-c2ccccc2S5)cc1] | 680.2 | 71 |
| Sub-h12 | CAS: 1361990-81-9 | 819 | | |
| 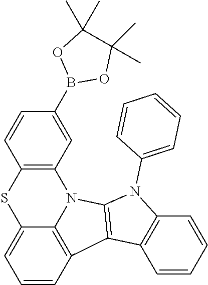 [SMILES: CC1(C)OB(c2ccc3c(c2)-n2c4c(cccc4c4c5ccccc5n(-c5ccccc5)c42)S3)OC1(C)C] | 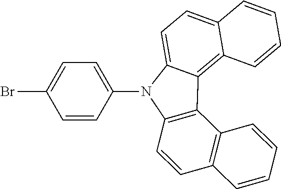 [SMILES: Brc1ccc(-n2c3ccc4ccccc4c3c3c4ccccc4ccc32)cc1] | 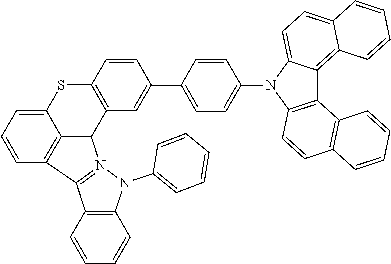 | 730.2 | 69 |
| Sub-f2 | CAS: 1419864-64-4 | 836 | | |

### Synthesis of Compound 349

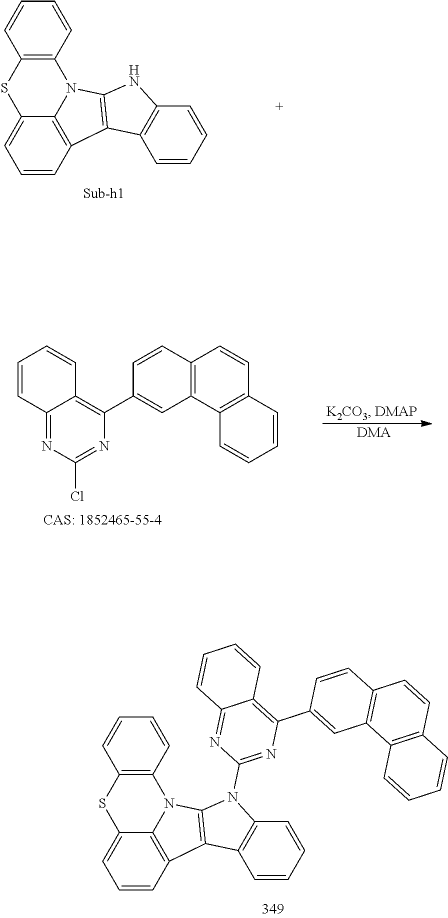

Sub-h1 (15.60 g, 50 mmol), reactant CAS: 1852465-55-4 (20.4 g, 60 mmol), anhydrous potassium carbonate (K₂CO₃, 6.91 g, 50 mmol), toluene (180 mL), dimethylaminopyridine (DMAP, 3.05 g, 25 mmol), and N,N-dimethylacetamide (DMA, 160 mL) were added sequentially to a 500-mL three-necked flask under nitrogen atmosphere, heated to 220° C. and stirred for 12 hours. After being cooled to room temperature, the resulting solution was extracted with ethyl acetate (100 mL×3 times). The combined organic phases were dried over anhydrous magnesium sulfate, filtered and distilled under reduced pressure to remove the solvent, obtaining a crude product. The crude product was purified by silica gel column chromatography with n-heptane/ethyl acetate as a mobile phase, yielding white solid Compound 349 (16.33 g, yield 53%), m/z=617.2 [M+H]⁺.

The following compounds of the present disclosure were synthesized respectively by following the synthesis of Compound 349, with reactant CAS: 1852465-55-4 being replaced with a corresponding reactant T shown in Table 12.

TABLE 12

| | Compound of the present | m/z | |
|---|---|---|---|
| Reactant T | disclosure | ([M + H]⁺ | Yield % |
| 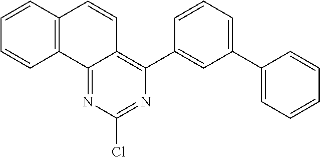 [SMILES: Clc1nc(-c2cccc(-c3ccccc3)c2)c2ccc3ccccc3c2n1] | 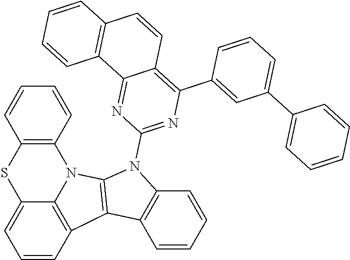 [SMILES: c1ccc(-c2cccc(-c3nc(-n4c5ccccc5c5c6cccc7c6n(c54)-c4ccccc4S7)nc4c3ccc3ccccc34)c2)cc1] | 643.2 | 48 |
| CAS: 1821393-79-6 | 353 | | |
| 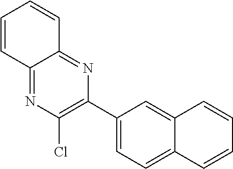 [SMILES: Clc1nc2ccccc2nc1-c1ccc2ccccc2c1] | 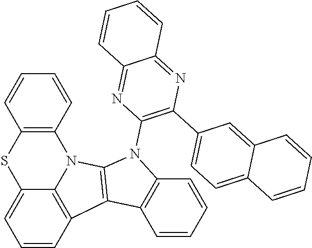 [SMILES: c1ccc2c(c1)Sc1cccc3c4c5ccccc5n(-c5nc6ccccc6nc5-c5ccc6ccccc6c5)c4n-2c13] | 567.2 | 55 |
| CAS: 955938-18-8 | 362 | | |
| 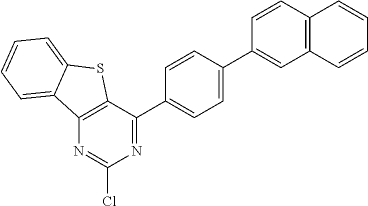 [SMILES: Clc1nc(-c2ccc(-c3ccc4ccccc4c3)cc2)c2sc3ccccc3c2n1] | 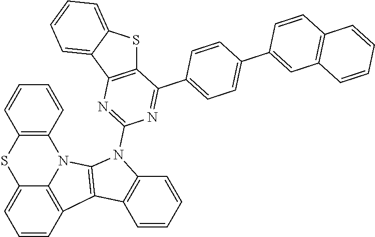 [SMILES: c1ccc2c(c1)Sc1cccc3c4c5ccccc5n(-c5nc(-c6ccc(-c7ccc8ccccc8c7)cc6)c6sc7ccccc7c6n5)c4n-2c13] | 699.2 | 53 |
| CAS: 1821152-09-3 | 370 | | |

### Synthesis of Compound 803

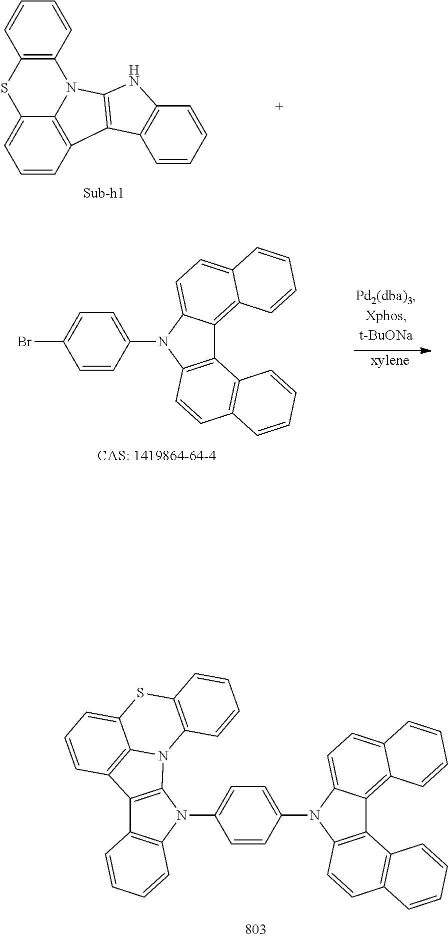 [SMILES: c1ccc2c(c1)Sc1cccc3c4c5ccccc5[nH]c4n-2c13 ; Brc1ccc(-n2c3ccc4ccccc4c3c3c4ccccc4ccc32)cc1 ; "xylene" ; c1ccc2c(c1)Sc1cccc3c4c5ccccc5n(-c5ccc(-n6c7ccc8ccccc8c7c7c8ccccc8ccc76)cc5)c4n-2c13]

Reactant Sub-h1 (7.81 g, 25 mmol), reactant CAS: 1419864-64-4 (11.61 g, 27.5 mmol), tris(dibenzylideneacetonyl)bis-palladium (Pd₂(dba)₃, 0.46 g, 0.5 mmol), 2-dicyclohexylphosphino-2′,4′,6′-triisopropylbiphenyl (X-Phos, 0.48 g, 1 mmol), sodium tert-butoxide (t-BuONa, 4.8 g, 50 mmol), and xylene (120 mL) were sequentially added to a 250-mL three-necked flask under a nitrogen atmosphere, heated to reflux and stirred overnight. After being cooled to room temperature, the reaction solution was poured into 500 mL of deionized water, stirred thoroughly for 30 minutes, and filtered. The resulting filter cake was rinsed with deionized water to neutral, and then rinsed with absolute ethanol (200 mL), obtaining a crude product. The crude product was purified by silica gel column chromatography with n-heptane/dichloromethane as a mobile phase, yielding white solid Compound 803 (12.10 g; yield 74%).

The following Compound 815 of the present disclosure was synthesized by following the synthesis of Compound 803, with reactant CAS: 1419864-64-4 being replaced with reactant Q shown in Table 13.

TABLE 13

| | Compound of the present | m/z | |
|---|---|---|---|
| Reactant Q | disclosure | ([M + H]⁺) | Yield % |
| 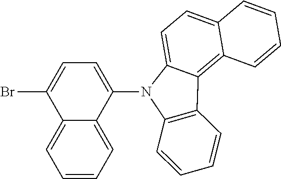 [SMILES: Brc1ccc(-n2c3ccccc3c3c4ccccc4ccc32)c2ccccc12] | 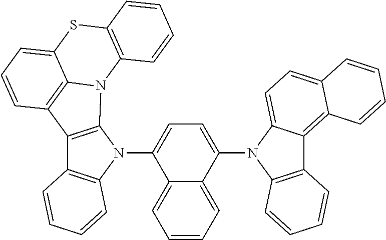 [SMILES: c1ccc2c(c1)Sc1cccc3c4c5ccccc5n(-c5ccc(-n6c7ccccc7c7c8ccccc8ccc76)c6ccccc56)c4n-2c13] | 654.2 | 68 |
| CAS: 1819346-89-8 | 815 | | |

NMR ¹HNMR data of the compounds of the present disclosure:

NMR data of Compound 7: ¹H-NMR (400 MHz, CD₂Cl₂) δ ppm 8.55-8.50 (m, 4H), 8.14 (d, 1H), 8.08 (d, 2H), 7.98 (d, 1H), 7.90 (d, 1H), 7.86 (d, 1H), 7.81-7.78 (m, 2H), 7.73 (d, 1H), 7.64 (d, 1H), 7.58 (t, 1H), 7.54-7.47 (m, 3H), 7.42 (t, 2H), 7.36 (d, 1H), 7.24 (t, 1H), 7.19-7.15 (m, 2H), 7.10 (d, 1H), 7.02 (t, 1H), 6.82 (d, 1H).

NMR data of Compound 730: ¹H-NMR (400 MHz, CD₂Cl₂) δ ppm 8.16 (d, 2H), 8.02 (d, 1H), 7.90 (d, 1H), 7.74 (d, 1H), 7.66-7.62 (m, 2H), 7.59-7.50 (m, 6H), 7.44 (d, 1H), 7.39-7.31 (m, 4H), 7.24 (t, 1H), 7.18-7.08 (m, 5H), 7.02 (t, 1H), 6.82 (d, 1H).

### Fabrication and Evaluation of Organic Electroluminescent Devices:

### Example 1: Fabrication of a Red Organic Electroluminescent Device

First, anode pretreatment was performed by the following processes. A surface of an ITO/Ag/ITO substrate, with thicknesses of ITO/Ag/ITO being 100 Å, 1000 Å, and 100 Å, respectively, was treated using ultraviolet ozone and O₂:N₂ plasma to increase the work function of the anode, and then cleaned with an organic solvent to remove impurities and oil thereon.

PD was deposited by vacuum evaporation on the experimental substrate (anode) to form a hole injection layer (HIL) with a thickness of 100 Å, and then α-NPD was deposited by vacuum evaporation on the hole injection layer to form a first hole transport layer with a thickness of 1080 Å.

Compound HT-1 was deposited by vacuum evaporation on the first hole transport layer to form a second hole transport layer with a thickness of 860 Å.

Next, Compound 3 and Ir(Mphq)₃ were co-deposited by evaporation on the second hole transport layer at an evaporation rate ratio of 98%:2% to form a red organic luminescent layer (EML) with a thickness of 400 Å.

Compound BTB and Compound LiQ were mixed at a 1:1 weight ratio and deposited by evaporation on the organic luminescent layer to form an electron transport layer (ETL) with a thickness of 350 Å; Yb was deposited by evaporation on the electron transport layer to form an electron injection layer (EIL) with a thickness of 10 Å; and then magnesium (Mg) and silver (Ag) were mixed at an evaporation rate ratio of 1:9, and deposited by vacuum evaporation on the electron injection layer to form a cathode with a thickness of 130 Å.

Further, CP-1 was deposited by vacuum evaporation on the above cathode to form a capping layer with a thickness of 800 Å, completing the fabrication of a red organic electroluminescent device.

### Examples 2 to 43

Organic electroluminescent devices were fabricated respectively by the same method as used in Example 1, except that Compound 3 in Example 1 was replaced with a corresponding one of the rest of the compounds X shown in the following Table 14 when an organic luminescent layer (EML) was formed.

### Comparative Examples 1 to 3

Organic electroluminescent devices were fabricated respectively by the same method as used in Example 1, except that Compound 3 in Example 1 was replaced with a corresponding one of Compound A, Compound B, or Compound C when an organic luminescent layer (EML) was formed.

Structures of the main materials used in various Examples and Comparative Examples are as follows:

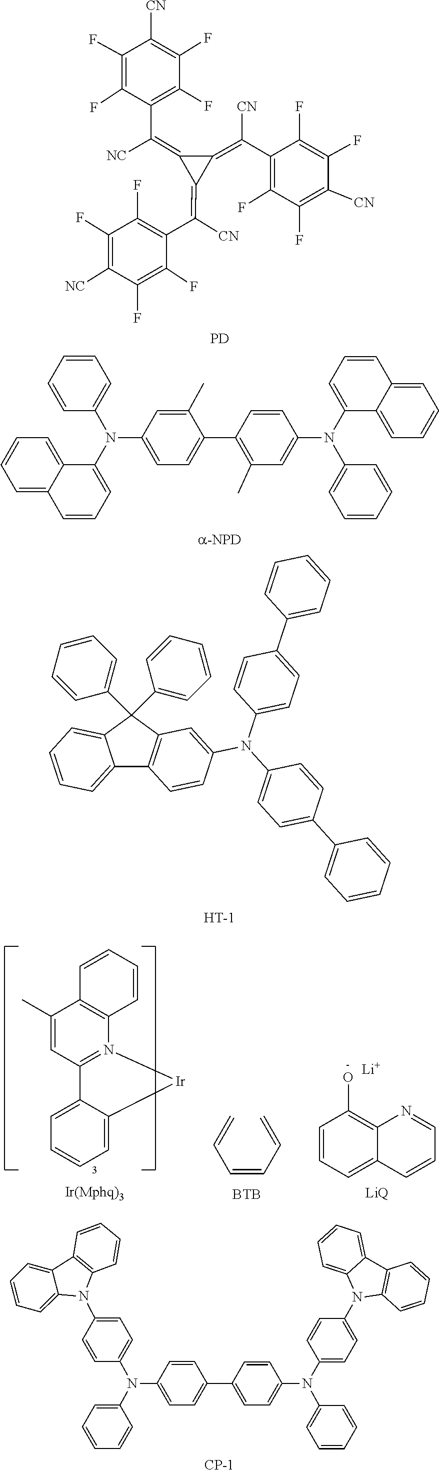

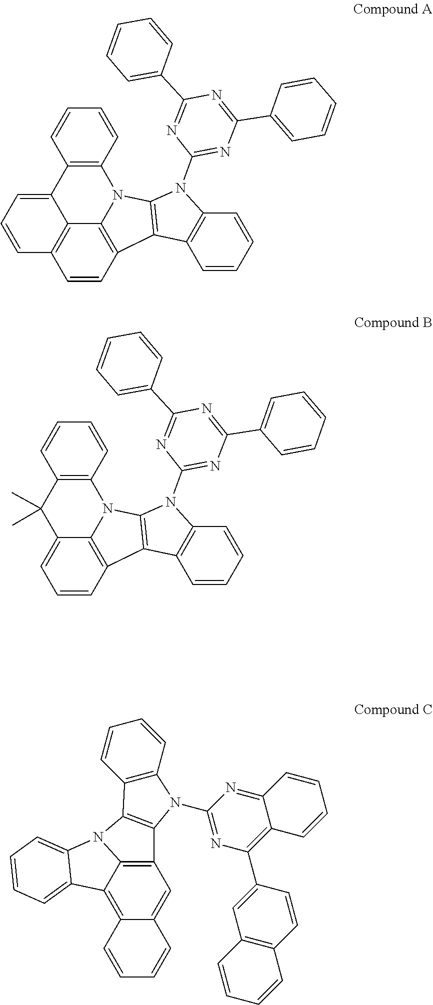 [SMILES: c1ccc(-c2nc(-c3ccccc3)nc(-n3c4ccccc4c4c5ccc6cccc7c8ccccc8n(c5c67)c43)n2)cc1 ; CC1(C)c2ccccc2-n2c3c1cccc3c1c3ccccc3n(-c3nc(-c4ccccc4)nc(-c4ccccc4)n3)c12 ; c1ccc2cc(-c3nc(-n4c5ccccc5c5c4c4cc6ccccc6c6c7ccccc7n5c46)nc4ccccc34)ccc2c1]

The red organic electroluminescent devices fabricated in Examples 1 to 43 and Comparative Examples 1 to 3 were tested for their performance. Specifically, the IVL characteristics of the devices were tested under the condition of 10 mA/cm², and the T95 lifetime of the devices was tested under the condition of 20 mA/cm². Test results are shown in Table 14.

TABLE 14

| | luminescent | | | | | |
|---|---|---|---|---|---|---|
| | layer = | Operating | | | | |
| | Compound X: | Voltage | | | | T95(h) |
| Example No. | Ir(Mphq)3 | (V) | Cd/A | CIEx | CIEy | @20 mA/cm² |
| Example 1 | Compound 3 | 3.42 | 59.6 | 0.680 | 0.320 | 499 |
| Example 2 | Compound 7 | 3.41 | 59.9 | 0.680 | 0.320 | 493 |
| Example 3 | Compound 10 | 3.45 | 59.5 | 0.680 | 0.320 | 491 |
| Example 4 | Compound 12 | 3.49 | 58.5 | 0.680 | 0.320 | 497 |
| Example 5 | Compound 22 | 3.52 | 58.2 | 0.680 | 0.320 | 503 |
| Example 6 | Compound 27 | 3.50 | 58.4 | 0.680 | 0.320 | 496 |
| Example 7 | Compound 29 | 3.49 | 58.6 | 0.680 | 0.320 | 495 |
| Example 8 | Compound 33 | 3.47 | 59.2 | 0.680 | 0.320 | 496 |
| Example 9 | Compound 42 | 3.43 | 59.5 | 0.680 | 0.320 | 488 |
| Example 10 | Compound 58 | 3.51 | 60.2 | 0.680 | 0.320 | 498 |
| Example 11 | Compound 64 | 3.48 | 58.5 | 0.680 | 0.320 | 499 |
| Example 12 | Compound 70 | 3.49 | 60.3 | 0.680 | 0.320 | 494 |
| Example 13 | Compound 75 | 3.52 | 59.8 | 0.680 | 0.320 | 506 |
| Example 14 | Compound 80 | 3.46 | 59.9 | 0.680 | 0.320 | 502 |
| Example 15 | Compound 100 | 3.45 | 59.5 | 0.680 | 0.320 | 505 |
| Example 16 | Compound 101 | 3.49 | 59.6 | 0.680 | 0.320 | 498 |
| Example 17 | Compound 120 | 3.47 | 60.1 | 0.680 | 0.320 | 507 |
| Example 18 | Compound 132 | 3.42 | 59.6 | 0.680 | 0.320 | 495 |
| Example 19 | Compound 187 | 3.47 | 59.2 | 0.680 | 0.320 | 494 |
| Example 20 | Compound 178 | 3.51 | 58.9 | 0.680 | 0.320 | 491 |
| Example 21 | Compound 201 | 3.50 | 55.2 | 0.680 | 0.320 | 553 |
| Example 22 | Compound 209 | 3.50 | 55.3 | 0.680 | 0.320 | 557 |
| Example 23 | Compound 212 | 3.47 | 55.2 | 0.680 | 0.320 | 549 |
| Example 24 | Compound 229 | 3.47 | 55.7 | 0.680 | 0.320 | 554 |
| Example 25 | Compound 235 | 3.51 | 56.0 | 0.680 | 0.320 | 550 |
| Example 26 | Compound 263 | 3.45 | 55.8 | 0.680 | 0.320 | 548 |
| Example 27 | Compound 272 | 3.43 | 55.8 | 0.680 | 0.320 | 546 |
| Example 28 | Compound 283 | 3.45 | 55.1 | 0.680 | 0.320 | 547 |
| Example 29 | Compound 292 | 3.46 | 56.1 | 0.680 | 0.320 | 542 |
| Example 30 | Compound 301 | 3.50 | 55.2 | 0.680 | 0.320 | 548 |
| Example 31 | Compound 349 | 3.46 | 59.2 | 0.680 | 0.320 | 492 |
| Example 32 | Compound 353 | 3.46 | 59.3 | 0.680 | 0.320 | 489 |
| Example 33 | Compound 362 | 3.45 | 59.4 | 0.680 | 0.320 | 500 |
| Example 34 | Compound 370 | 3.49 | 59.3 | 0.680 | 0.320 | 499 |
| Example 35 | Compound 377 | 3.52 | 59.3 | 0.680 | 0.320 | 498 |
| Example 36 | Compound 382 | 3.50 | 59.5 | 0.680 | 0.320 | 493 |
| Example 37 | Compound 415 | 3.45 | 56.7 | 0.680 | 0.320 | 551 |
| Example 38 | Compound 421 | 3.47 | 56.4 | 0.680 | 0.320 | 545 |
| Example 39 | Compound 424 | 3.46 | 56.0 | 0.680 | 0.320 | 540 |
| Example 40 | Compound 468 | 3.49 | 56.5 | 0.680 | 0.320 | 450 |
| Example 41 | Compound 473 | 3.49 | 56.3 | 0.680 | 0.320 | 449 |
| Example 42 | Compound 499 | 3.51 | 56.5 | 0.680 | 0.320 | 451 |
| Example 43 | Compound 583 | 3.46 | 56.4 | 0.680 | 0.320 | 453 |
| Comparative | Compound A | 3.55 | 49.7 | 0.680 | 0.320 | 396 |
| Example 1 | | | | | | |
| Comparative | Compound B | 3.52 | 46.7 | 0.680 | 0.320 | 392 |
| Example 2 | | | | | | |
| Comparative | Compound C | 3.55 | 49.0 | 0.680 | 0.320 | 404 |
| Example 3 | | | | | | |

As can be seen from Table 14, compared with Comparative Examples 1 to 3, the Examples in which the compounds of the present disclosure are used as a red light-emitting host material of an organic electroluminescent device improve the efficiency of the device by at least 10.8% and by at most 29.1%, and prolong the service life thereof by at least 11.1% and by at most 42.1%, while a relatively low operating voltage is maintained.

### Example 44: A Red Organic Electroluminescent Device

First, anode pretreatment was performed by the following processes. A surface of an ITO/Ag/ITO substrate, with thicknesses of ITO/Ag/ITO being 100 Å, 1000 Å, and 100 Å, respectively, was treated using ultraviolet ozone and O₂:N₂ plasma to increase the work function of the anode, and then cleaned with an organic solvent to remove impurities and oil thereon.

PD was deposited by vacuum evaporation on the experimental substrate (anode) to form a hole injection layer (HIL) with a thickness of 100 Å, and then α-NPD was deposited by vacuum evaporation on the hole injection layer to form a first hole transport layer with a thickness of 1080 Å.

Compound HT-1 was deposited by vacuum evaporation on the first hole transport layer to form a second hole transport layer with a thickness of 890 Å.

Next, Compound RH—N, Compound 650, and Ir(Mphq)₃ were co-deposited by evaporation on the second hole transport layer at an evaporation rate ratio of 49%:49%:2% to form a red organic luminescent layer (EML) with a thickness of 400 Å.

Compound BTB and Compound LiQ were mixed at a 1:1 weight ratio and deposited by evaporation on the organic luminescent layer to form an electron transport layer (ETL) with a thickness of 350 Å; Yb was deposited by evaporation on the electron transport layer to form an electron injection layer (EIL) with a thickness of 10 Å; and then magnesium (Mg) and silver (Ag) were mixed at an evaporation rate ratio of 1:9, and deposited by vacuum evaporation on the electron injection layer to form a cathode with a thickness of 130 Å.

Further, Compound CP-1 was deposited at a thickness of 800 Å by vacuum evaporation on the above cathode, completing the fabrication of a red-light emitting organic electroluminescent device.

### Examples 45 to 56

Organic electroluminescent devices were fabricated respectively by the same method as used in Example 44, except that Compound 650 in Example 44 was replaced with a corresponding compound Y shown in the following Table 15 when an organic luminescent layer was formed.

### Comparative Examples 4 to 5

Organic electroluminescent devices were fabricated respectively by the same method as used in Example 44, except that Compound 650 in Example 44 was replaced with a corresponding one of Compound D and Compound E when an organic luminescent layer was formed.

Structures of the main materials used in Examples 44 to 56 and Comparative Examples 4 to 5 are as follows:

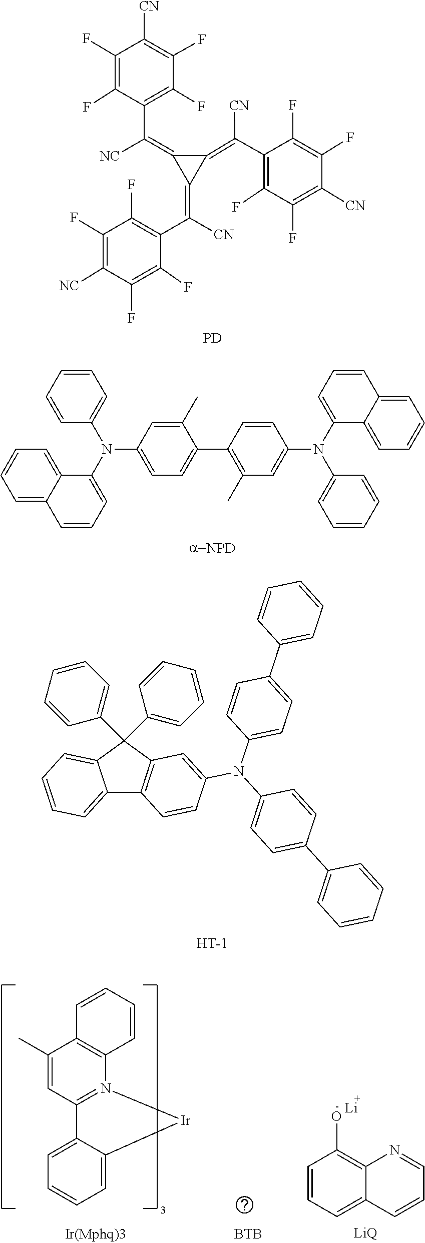

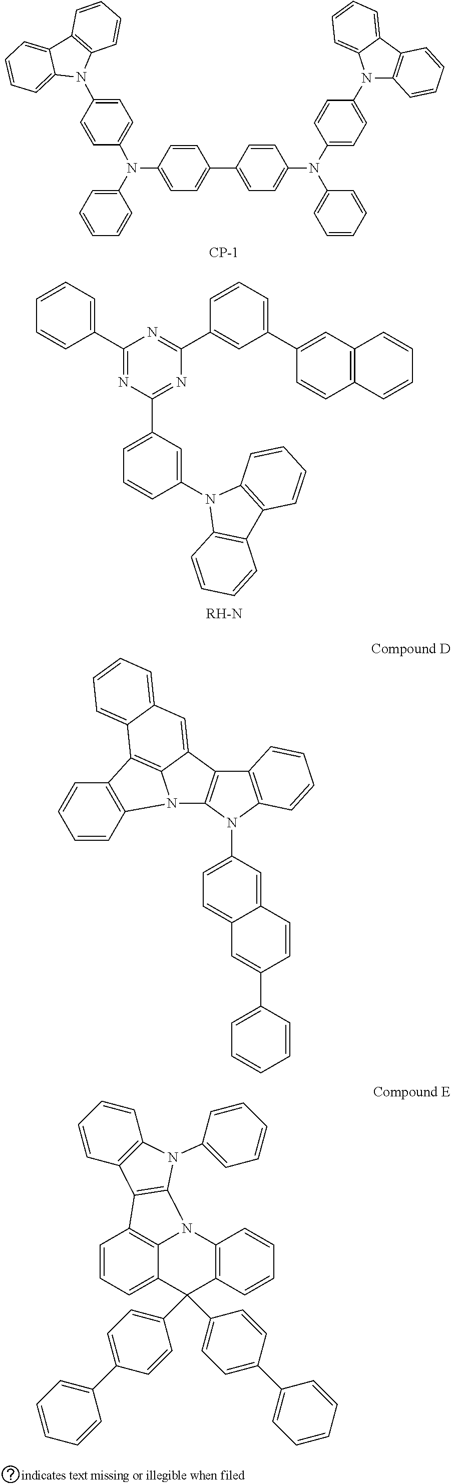

The red organic electroluminescent devices fabricated in Examples 44 to 56 and Comparative Examples 4 to 5 were tested for their performance. Specifically, the IVL characteristics of the devices were tested under the condition of 10 mA/cm², and the T95 lifetime of the devices was tested under the condition of 20 mA/cm². Test results are shown in Table 15.

TABLE 15

| | luminescent | | | | | |
|---|---|---|---|---|---|---|
| | layer = | Operating | | | | |
| | RH-N: Compounds: | Voltage | | | | T95(hrs) |
| Example No. | Ir(Mphq)3 | (V) | Cd/A | CIEx | CIEy | @20 mA/cm² |
| Example 44 | Compound 650 | 3.38 | 67.2 | 0.680 | 0.320 | 439 |
| Example 45 | Compound 668 | 3.40 | 67.0 | 0.680 | 0.320 | 447 |
| Example 46 | Compound 709 | 3.40 | 60.7 | 0.680 | 0.320 | 406 |
| Example 47 | Compound 716 | 3.39 | 61.9 | 0.680 | 0.320 | 486 |
| Example 48 | Compound 730 | 3.42 | 66.8 | 0.680 | 0.320 | 445 |
| Example 49 | Compound 758 | 3.47 | 66.3 | 0.680 | 0.320 | 448 |
| Example 50 | Compound 695 | 3.46 | 66.7 | 0.680 | 0.320 | 439 |
| Example 51 | Compound 707 | 3.41 | 61.3 | 0.680 | 0.320 | 489 |
| Example 52 | Compound 705 | 3.40 | 66.9 | 0.680 | 0.320 | 441 |
| Example 53 | Compound 803 | 3.41 | 66.5 | 0.680 | 0.320 | 451 |
| Example 54 | Compound 815 | 3.43 | 67.1 | 0.680 | 0.320 | 452 |
| Example 55 | Compound 819 | 3.42 | 61.7 | 0.680 | 0.320 | 416 |
| Example 56 | Compound 836 | 3.37 | 61.6 | 0.680 | 0.320 | 492 |
| Comparative | Compound D | 3.48 | 48.1 | 0.680 | 0.320 | 350 |
| Example 4 | | | | | | |
| Comparative | Compound E | 3.52 | 41.3 | 0.680 | 0.320 | 337 |
| Example 5 | | | | | | |

As can be seen from Table 15 above, the compounds of the present disclosure, when used as a hole transport-type host material in a mixed-type host material of a red organic electroluminescent device, improve the efficiency of the device by at least 26.2%, and prolong the service life thereof by at least 16% and by at most 45.1%, while a relatively low operating voltage is maintained.

The structures of the compounds of the present disclosure each have a core structure that is indolyl-fused phenothiazine/phenoxazine. The sulfur or oxygen atom in indolophenothiazine/indolophenoxazine has two pairs of lone pair electrons, which can give the core structure an excellent hole transport ability. When the core structure is linked with an aryl group or an electron-rich heteroaryl group, the compound can have an enhanced hole transport ability; and this type of compounds are suitable for use as a hole transport-type host material in a mixed-type host material.

When the core structure is linked with a nitrogen-containing heteroarylene group having electron transport characteristics, the compound can have both excellent hole transport performance and excellent electron transport performance; and this type of compounds are suitable for use as a single-type host material. The compounds of the present disclosure, whenever used as a hole transport-type host material in a mixed-type host material or as a single-type host material, can improve the balance of charge carriers in luminescent layer, expand a region where the carriers recombine, improve the generation and utilization efficiency of excitons as well as the luminescence efficiency and the service life of a device.

1. A nitrogen-containing compound, having a structure shown in Formula 1: 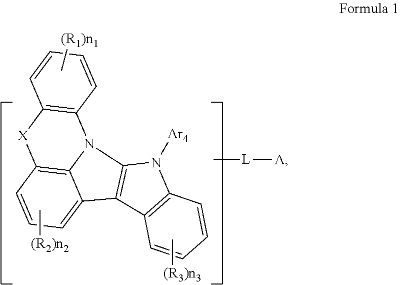
wherein:
X is selected from S or O;
group A is selected from a structure shown in Formula a-1 or a structure shown in Formula 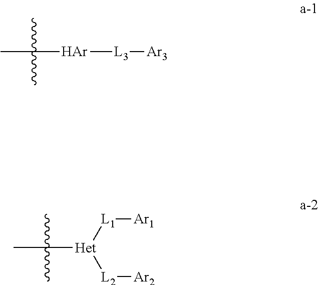
HAr is selected from substituted or unsubstituted arylene having 6 to 40 carbon atoms, and substituted or unsubstituted heteroarylene having 3 to 40 carbon atoms;
Het is a nitrogen-containing heteroarylene having 3 to 20 carbon atoms;
substituents in HAr are identical or different, and are each independently selected from the group consisting of deuterium, cyano, halogen, alkyl having 1 to 10 carbon atoms, deuterated alkyl having 1 to 10 carbon atoms, and aryl having 6 to 20 carbon atoms;
L, L₁, L₂, and L₃ are identical or different, and are each independently selected from the group consisting of single bond, substituted or unsubstituted arylene having 6 to 30 carbon atoms, and substituted or unsubstituted heteroarylene having 3 to 30 carbon atoms;
Ar₁ and Ar₂ are identical or different, and are each independently selected from the group consisting of substituted or unsubstituted aryl having 6 to 40 carbon atoms, and substituted or unsubstituted heteroaryl having 3 to 40 carbon atoms;
Ar₃ is selected from the group consisting of hydrogen, substituted or unsubstituted aryl having 6 to 40 carbon atoms, and substituted or unsubstituted heteroaryl having 3 to 40 carbon atoms;
Ar₄ is selected from the group consisting of substituted or unsubstituted aryl having 6 to 40 carbon atoms, and substituted or unsubstituted heteroaryl having 3 to 40 carbon atoms, or Ar₄ is single bond;
substituents in L, L₁, L₂, L₃, Ar₁, Ar₂, Ar₃, and Ar₄ are identical or different, and are each independently selected from the group consisting of deuterium, cyano, halogen, alkyl having 1 to 10 carbon atoms, haloalkyl having 1 to 10 carbon atoms, deuterated alkyl having 1 to 10 carbon atoms, trialkylsilyl having 3 to 12 carbon atoms, triphenylsilyl, aryl having 6 to 20 carbon atoms, heteroaryl having 3 to 20 carbon atoms, phosphonyl having 6 to 20 carbon atoms, cycloalkyl having 3 to 10 carbon atoms, alkoxy having 1 to 10 carbon atoms, alkylthio having 1 to 10 carbon atoms, aryloxy having 6 to 20 carbon atoms, and arylthio having 6 to 20 carbon atoms; and optionally, in Ar₁, Ar₂, Ar₃, and Ar₄, any two adjacent substituents form a saturated or unsaturated 3 to 15-membered ring;
each R₁, each R₂, and each R₃ is identical or different, and is independently selected from the group consisting of deuterium, cyano, halogen, alkyl having 1 to 10 carbon atoms, haloalkyl having 1 to 10 carbon atoms, deuterated alkyl having 1 to 10 carbon atoms, trialkylsilyl having 3 to 12 carbon atoms, aryl having 6 to 20 carbon atoms, heteroaryl having 3 to 20 carbon atoms, and cycloalkyl having 3 to 10 carbon atoms; and optionally, any two adjacent groups form a benzene ring; and
n₁ represents the number of R₁, and is selected from 0, 1, 2, 3, or 4; n₂ represents the number of R₂, and is selected from 0, 1, 2, or 3; n₃ represents the number of R₃, and is selected from 0, 1, 2, 3, or 4.

2. The nitrogen-containing compound according to claim 1, wherein the nitrogen-containing compound shown in Formula 1 is selected from the following structures:

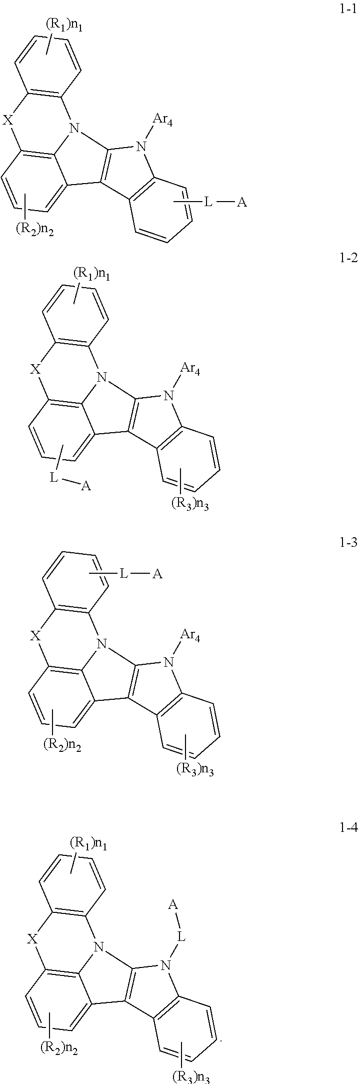

3. The nitrogen-containing compound according to claim 1, wherein Het is selected from triazinylene, pyrimidinylidene, or pyridylidene;
optionally, Het is selected from 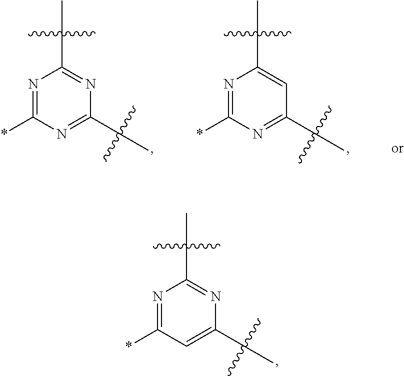
wherein [symbol] represents a bond linked with L, and [symbol] represents a bond linked with L₁ or L₂.

4. The nitrogen-containing compound according to claim 1, wherein HAr is selected from the group consisting of substituted or unsubstituted phenylene, substituted or unsubstituted naphthylene, substituted or unsubstituted biphenylene, substituted or unsubstituted anthrylene, substituted or unsubstituted phenanthrylene, substituted or unsubstituted fluorenylidene, substituted or unsubstituted spirobifluorenylidene, substituted or unsubstituted dibenzothienylene, substituted or unsubstituted dibenzofuranylene, and substituted or unsubstituted carbazolylene, or selected from the following substituted or unsubstituted groups: 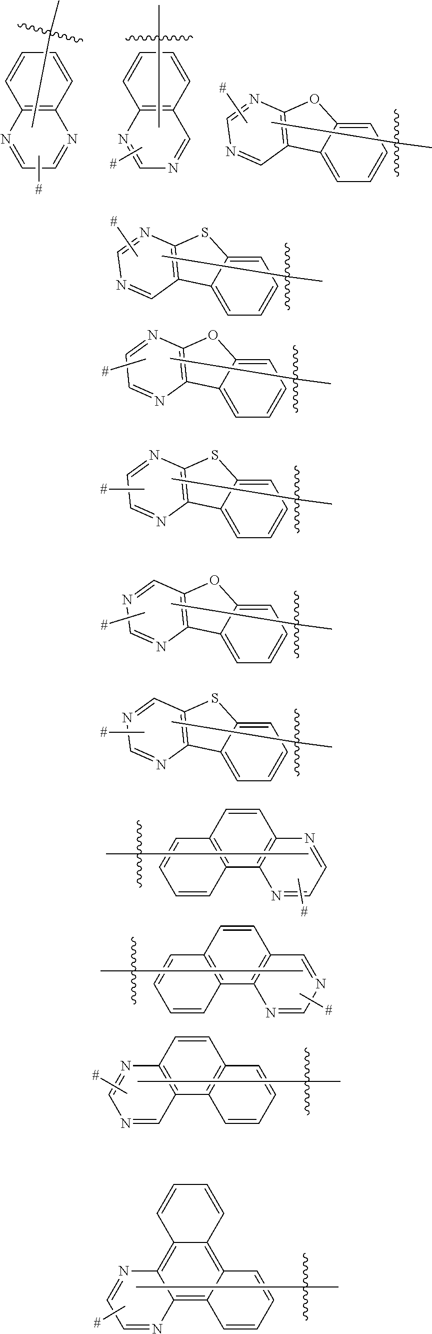 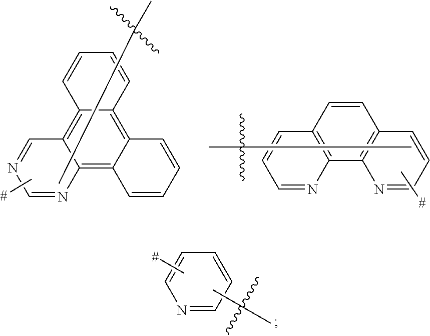
wherein [symbol] represents a bond linked with L, and [symbol] represents a bond linked with L₃; the substituents in HAr are each identical or different, and are each independently selected from the group consisting of deuterium, fluorine, cyano, trideuteromethyl, trifluoromethyl, and alkyl having 1 to 4 carbon atoms, and phenyl.

5. The nitrogen-containing compound according to claim 1, wherein HAr is selected from substituted or unsubstituted group W, wherein the unsubstituted group W is selected from the group consisting of the following groups: 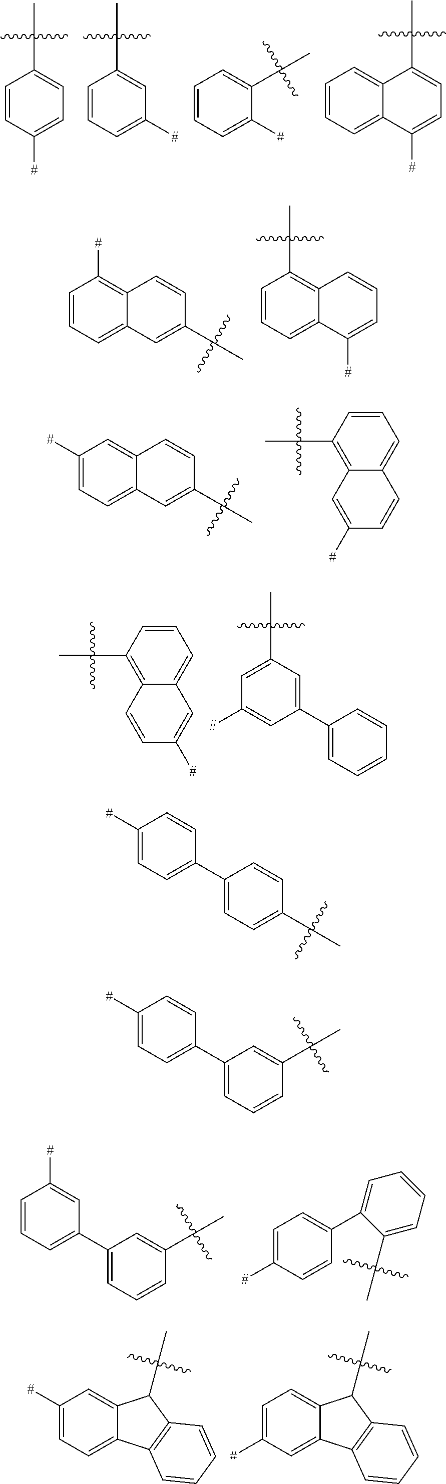 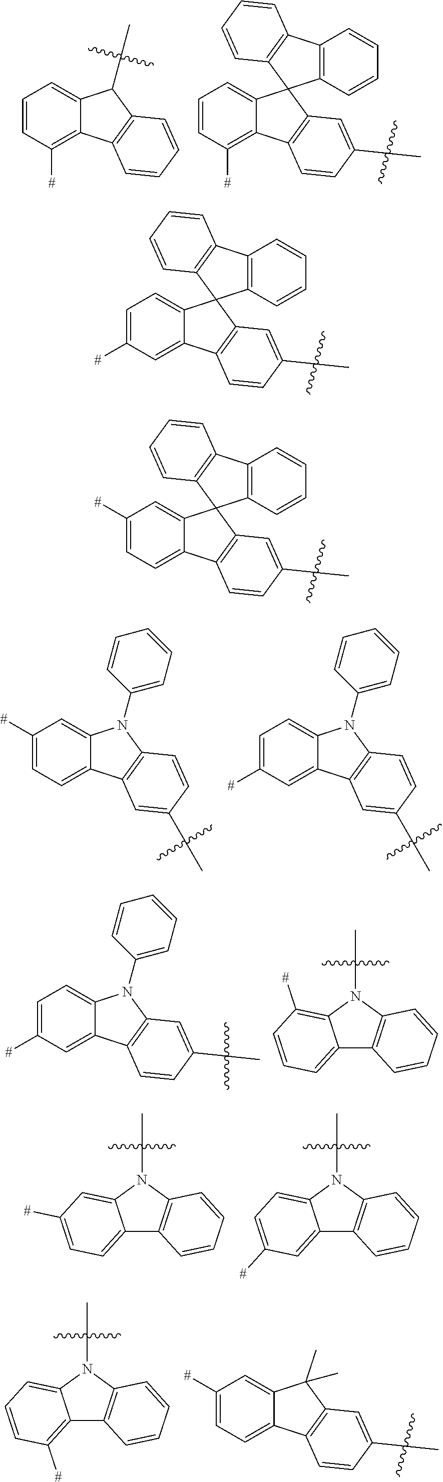 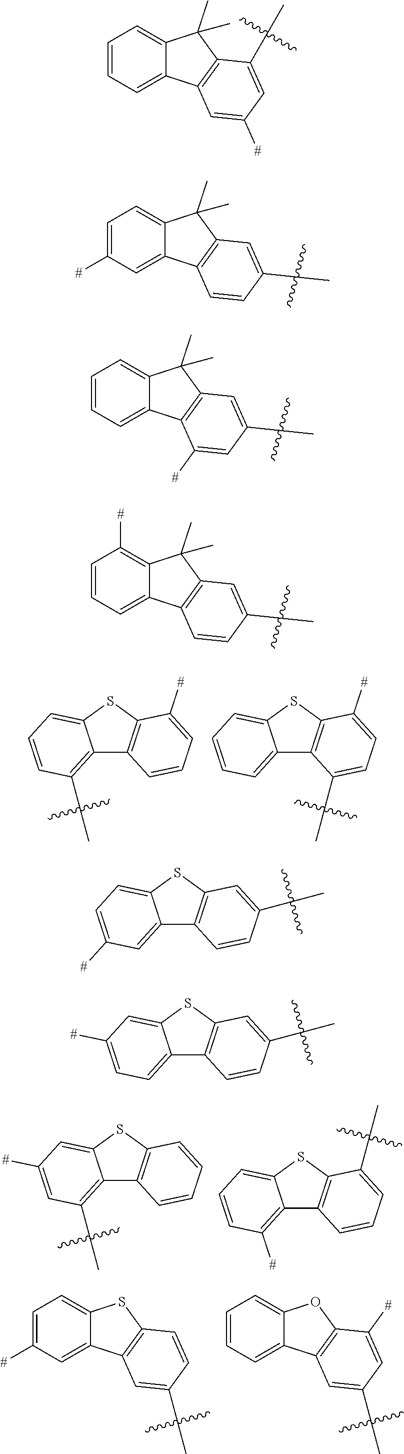 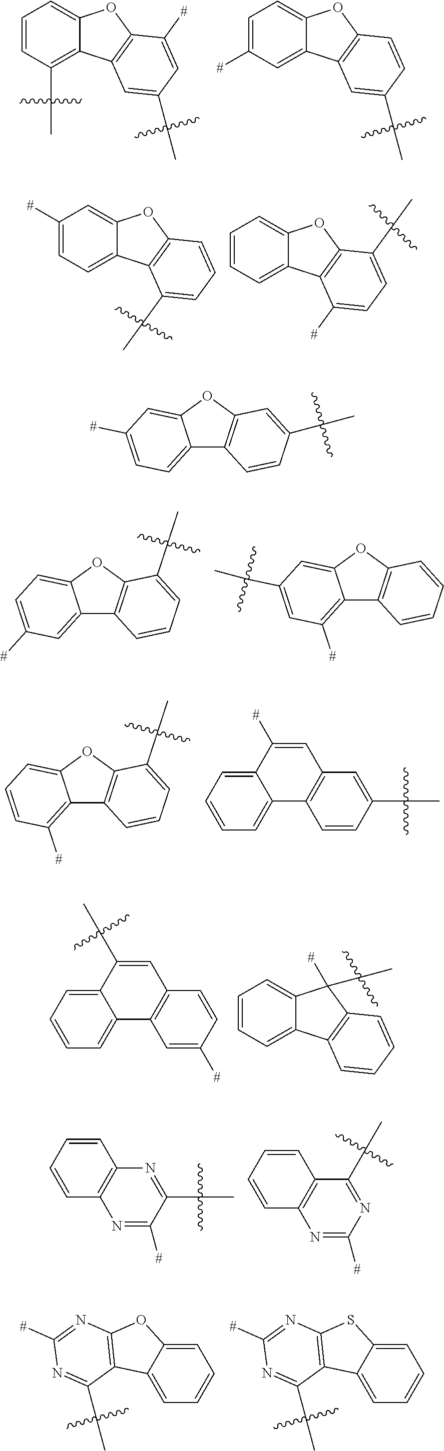 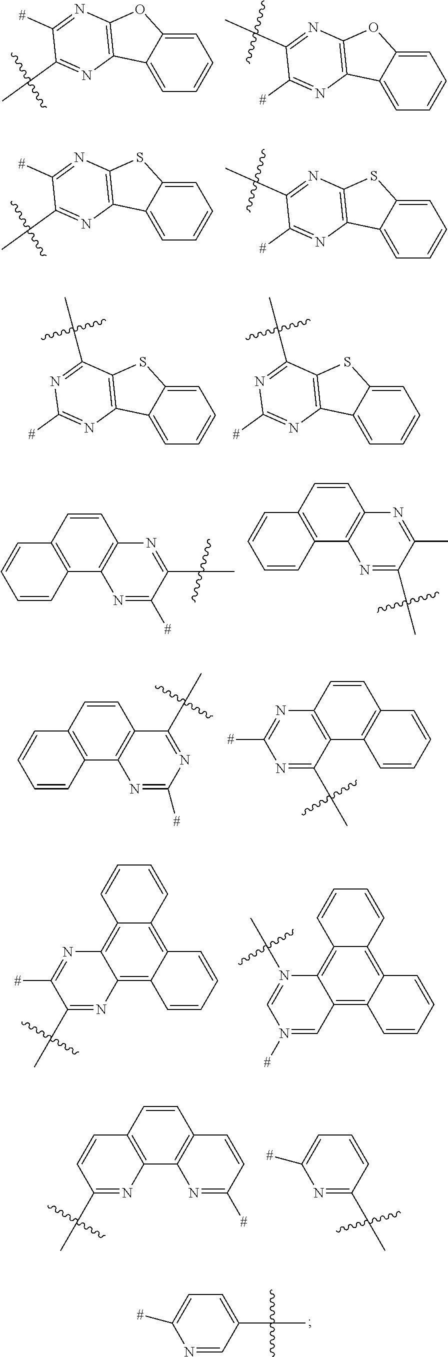
wherein [symbol] represents a bond linked with L, and [symbol] represents a bond linked with L₃;
the substituted group W is formed by the unsubstituted group W being substituted by one or more substituents, wherein the substituents are each independently selected from deuterium, fluorine, cyano, trideuteromethyl, trifluoromethyl, methyl, ethyl, isopropyl, tert-butyl, and phenyl, and when the number of the substituents is greater than 1, the substituents are identical or different.

6. The nitrogen-containing compound according to claim 1, wherein Ar₁ and Ar₂ are identical or different, and are each independently selected from substituted or unsubstituted aryl having 6 to 25 carbon atoms, and substituted or unsubstituted heteroaryl having 5 to 20 carbon atoms;
optionally, Ar₃ is selected from hydrogen, substituted or unsubstituted aryl having 6 to 25 carbon atoms, and substituted or unsubstituted heteroaryl having 5 to 20 carbon atoms;
optionally, Ar₄ is selected from single bond, substituted or unsubstituted aryl having 6 to 25 carbon atoms, and substituted or unsubstituted heteroaryl having 5 to 20 carbon atoms; and
optionally, the substituents in Ar₁, Ar₂, Ar₃, and Ar₄ are each independently selected from the group consisting of deuterium, halogen, cyano, haloalkyl having 1 to 4 carbon atoms, deuterated alkyl having 1 to 4 carbon atoms, alkyl having 1 to 4 carbon atoms, cycloalkyl having 5 to 10 atom groups, aryl having 6 to 12 carbon atoms, heteroaryl having 5 to 12 carbon atoms, and trialkylsilyl having 3 to 8 atom groups; and optionally, any two adjacent substituents form a benzene ring or a fluorene ring.

7. The nitrogen-containing compound according to claim 1, wherein Ar₁ and Ar₂ are identical or different, and are each independently selected from the group consisting of substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted spirobifluorenyl, substituted or unsubstituted triphenylene, substituted or unsubstituted pyrenyl, substituted or unsubstituted perylenyl, substituted or unsubstituted pyridyl, substituted or unsubstituted dibenzothienyl, substituted or unsubstituted dibenzofuranyl, substituted or unsubstituted carbazolyl, substituted or unsubstituted quinolyl, substituted or unsubstituted phenanthrolin, substituted or unsubstituted benzothiazolyl, substituted or unsubstituted benzoxazolyl, and substituted or unsubstituted benzimidazolyl; and
optionally, the substituents in Ar₁ and Ar₂ are each independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, pyridyl, dibenzofuranyl, dibenzothienyl, and carbazolyl; and optionally, in Ar₁ and Ar₂, any two adjacent substituents form a benzene ring.

8. The nitrogen-containing compound according to claim 1, wherein Ar₃ is selected from the group consisting of hydrogen, substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted spirobifluorenyl, substituted or unsubstituted triphenylene, substituted or unsubstituted pyrenyl, substituted or unsubstituted perylenyl, substituted or unsubstituted pyridyl, substituted or unsubstituted dibenzothienyl, substituted or unsubstituted dibenzofuranyl, substituted or unsubstituted carbazolyl, substituted or unsubstituted quinolyl; and
optionally, the substituents in Ar₃ are each independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, and pyridyl; and optionally, in Ar₃, any two adjacent substituents form a benzene ring.

9. The nitrogen-containing compound according to claim 1, wherein Ar₄ is selected from the group consisting of single bond, substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted spirobifluorenyl, substituted or unsubstituted triphenylene, substituted or unsubstituted pyrenyl, substituted or unsubstituted perylenyl, substituted or unsubstituted pyridyl, substituted or unsubstituted dibenzothienyl, substituted or unsubstituted dibenzofuranyl, and substituted or unsubstituted carbazolyl; and
optionally, the substituents in Ar₄ are each independently selected from the group consisting of deuterium, fluorine, cyano, trideuteromethyl, trimethylsilyl, trifluoromethyl, cyclopentyl, cyclohexyl, adamantly, methyl, ethyl, isopropyl, tert-butyl, phenyl, naphthyl, dibenzothienyl, dibenzofuranyl, and carbazolyl; and optionally, in Ar₄, any two adjacent substituents form a benzene ring.

10. The nitrogen-containing compound according to claim 1, wherein L and L₃ are identical or different, and are each independently selected from the group consisting of single bond, substituted or unsubstituted phenylene, substituted or unsubstituted naphthylene, and substituted or unsubstituted biphenylene;
optionally, L₁ and L₂ are identical or different, and are each independently selected from the group consisting of single bond, substituted or unsubstituted phenylene, substituted or unsubstituted naphthylene, substituted or unsubstituted biphenylene, substituted or unsubstituted fluorenylidene, substituted or unsubstituted phenanthrylene, substituted or unsubstituted dibenzothienylene, substituted or unsubstituted dibenzofuranylene, substituted or unsubstituted carbazolylene, and substituted or unsubstituted pyridylidene; and
optionally, the substituents in L, L₃, L₁, and L₂ are each independently selected from the group consisting of deuterium, fluorine, cyano, methyl, ethyl, isopropyl, tert-butyl, trifluoromethyl, trideuteromethyl, trimethylsilyl, and phenyl.

11. The nitrogen-containing compound according to claim 1, wherein each R₁, each R₂, and each R₃ is identical or different, and is independently selected from the group consisting of deuterium, cyano, fluorine, trimethylsilyl, trideuteromethyl, trifluoromethyl, cyclopentyl, cyclohexyl, methyl, ethyl, isopropyl, tert-butyl, phenyl, and naphthyl; and optionally, any two adjacent groups form a benzene ring.

12. The nitrogen-containing compound according to claim 1, wherein 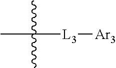
is selected from hydrogen or the group consisting of the following groups: 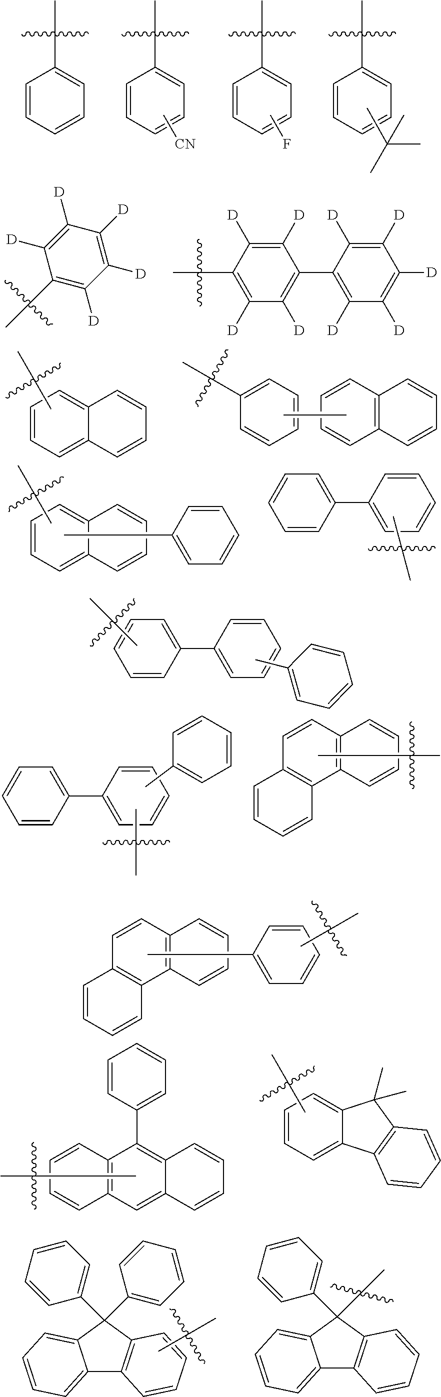 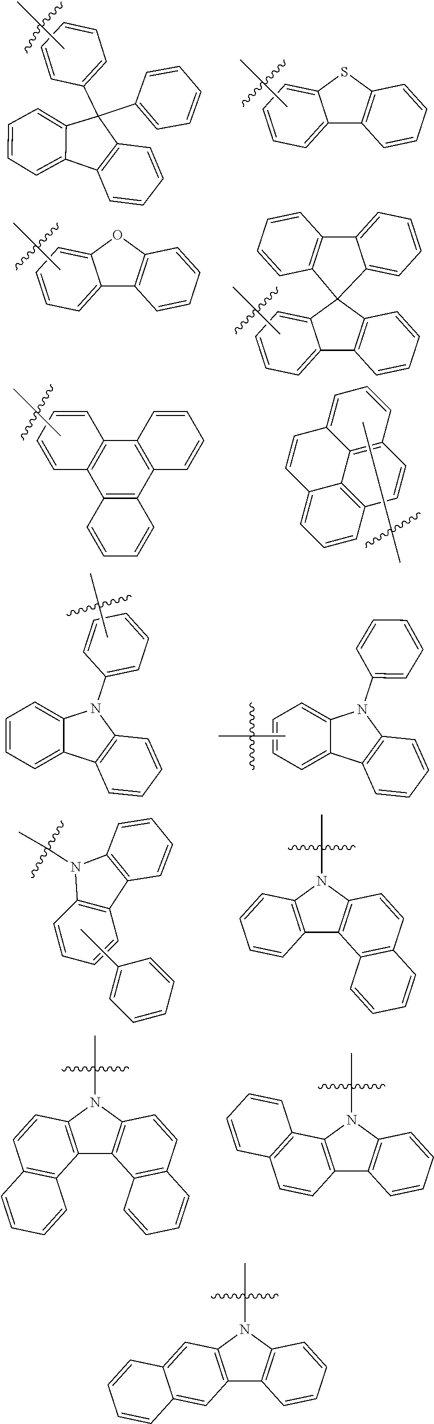 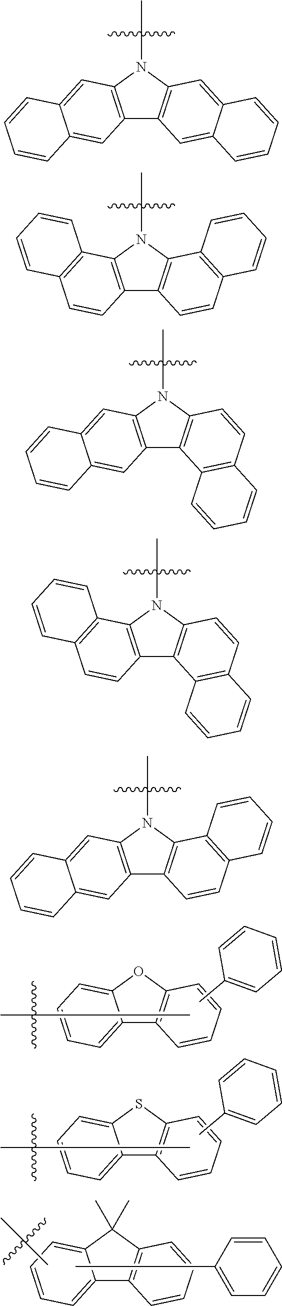 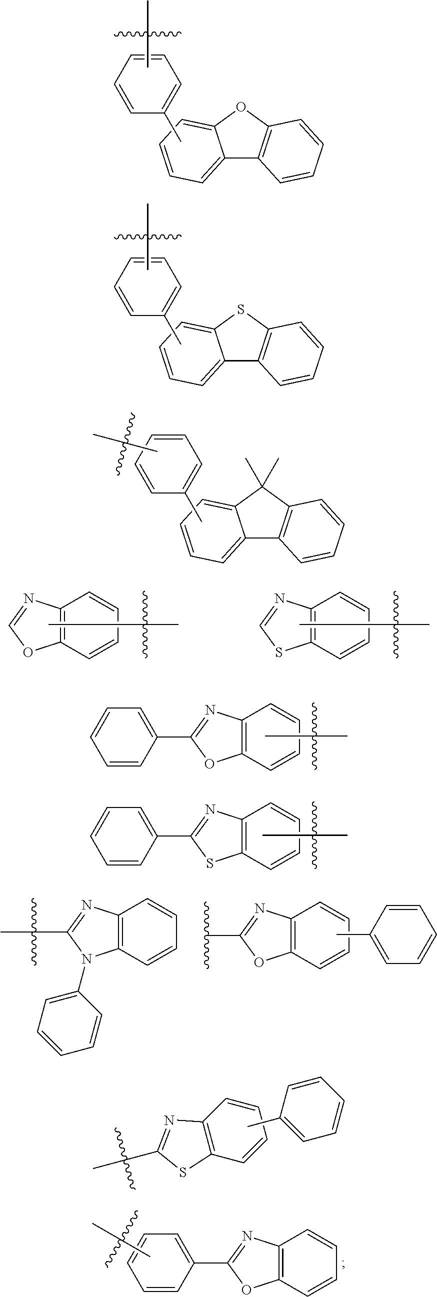
and
optionally, 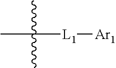
and 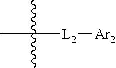
are each independently selected from the following groups:

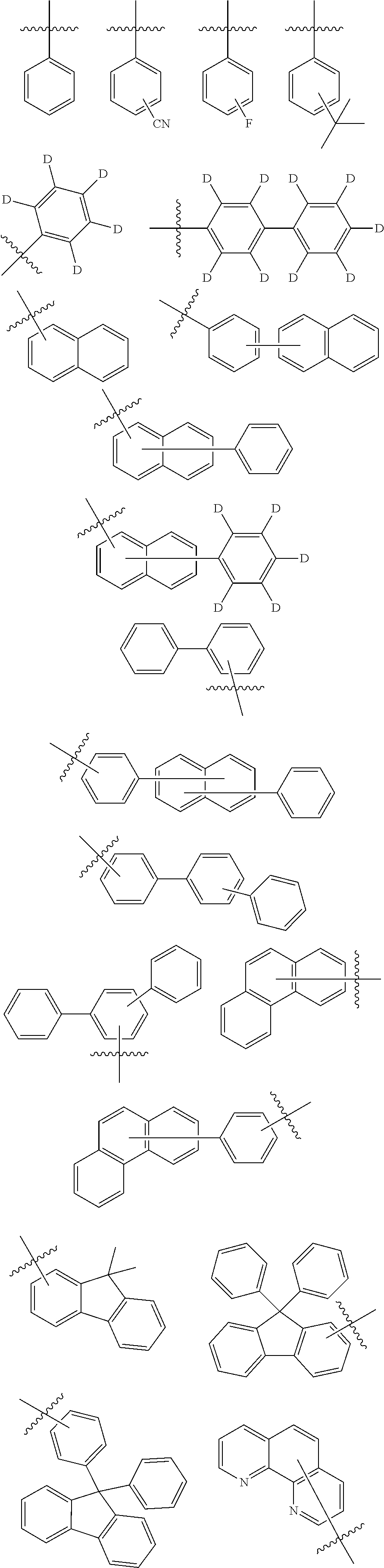

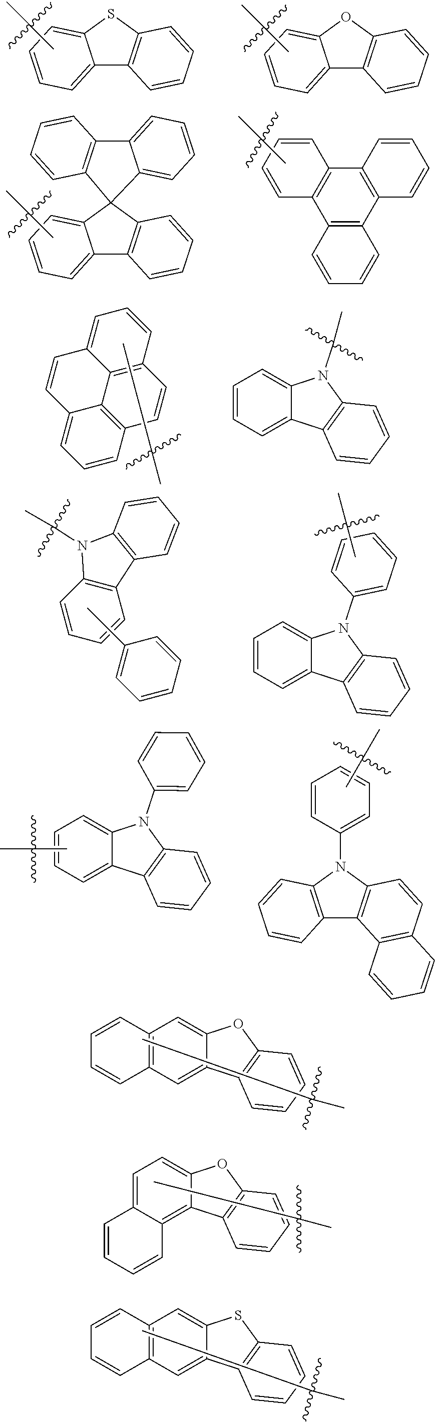

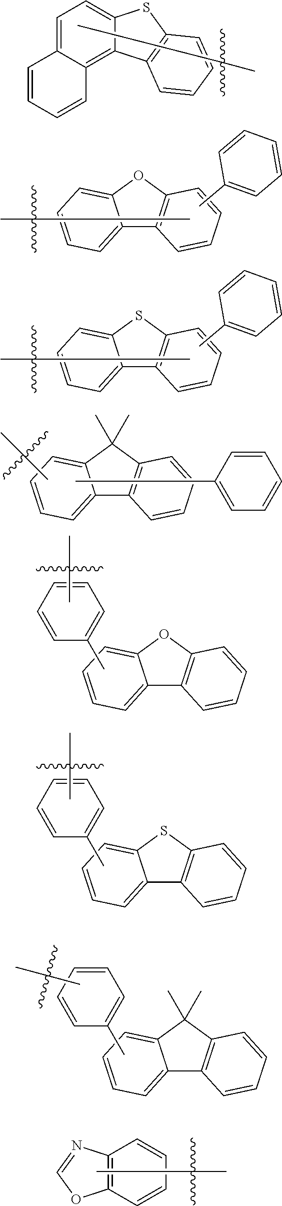

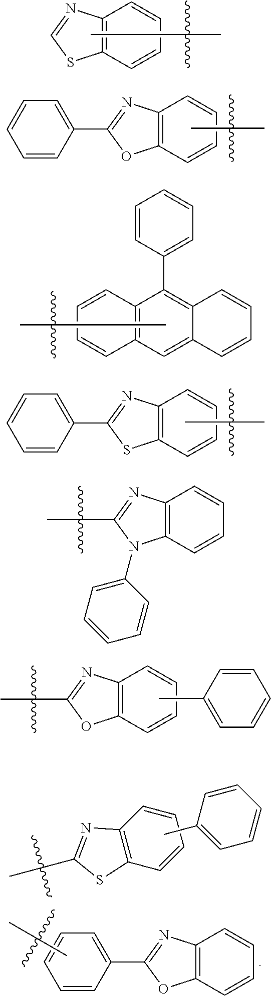

13. The nitrogen-containing compound according to claim 1, wherein group A is selected from the group consisting of the following groups:

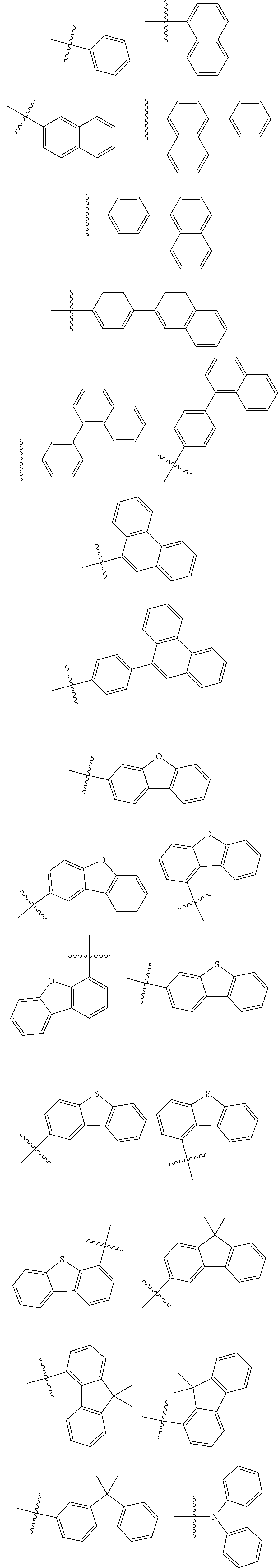

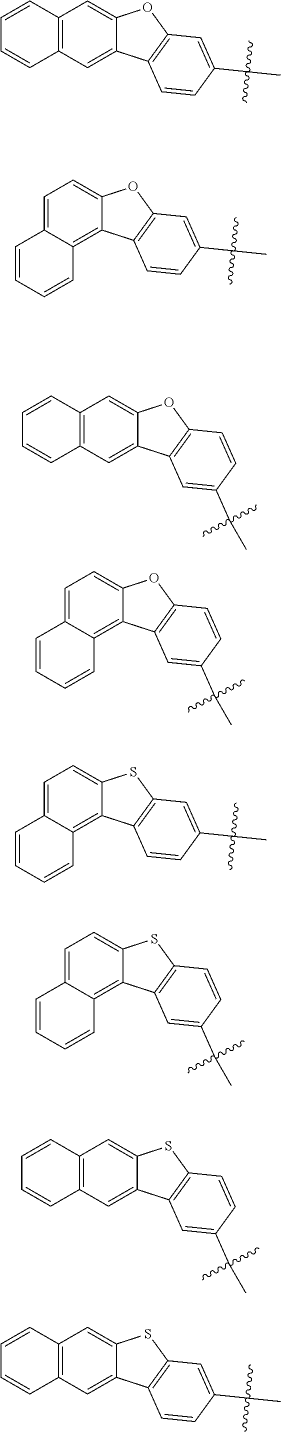

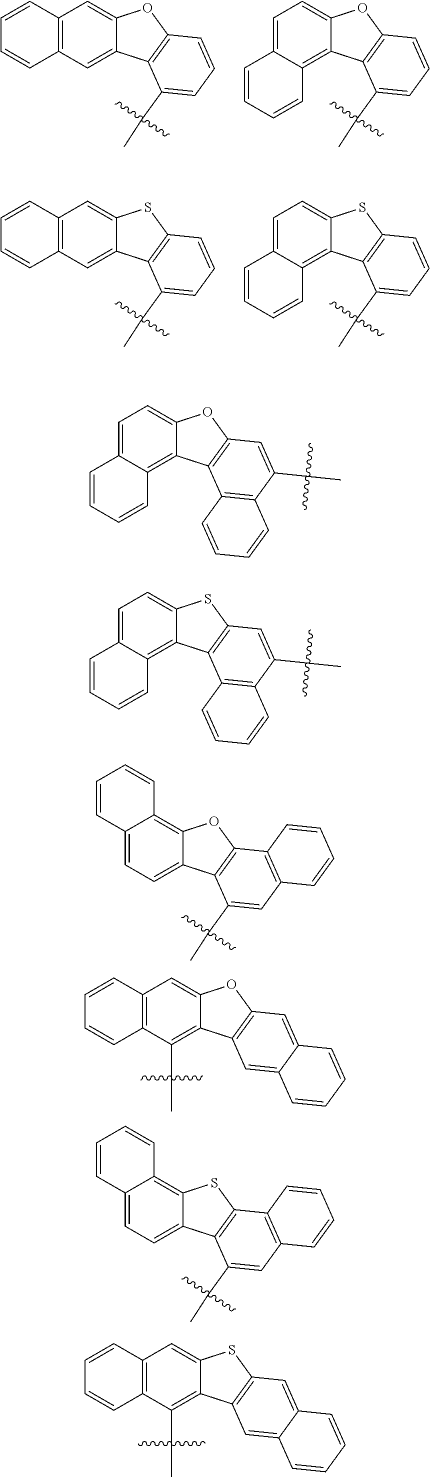

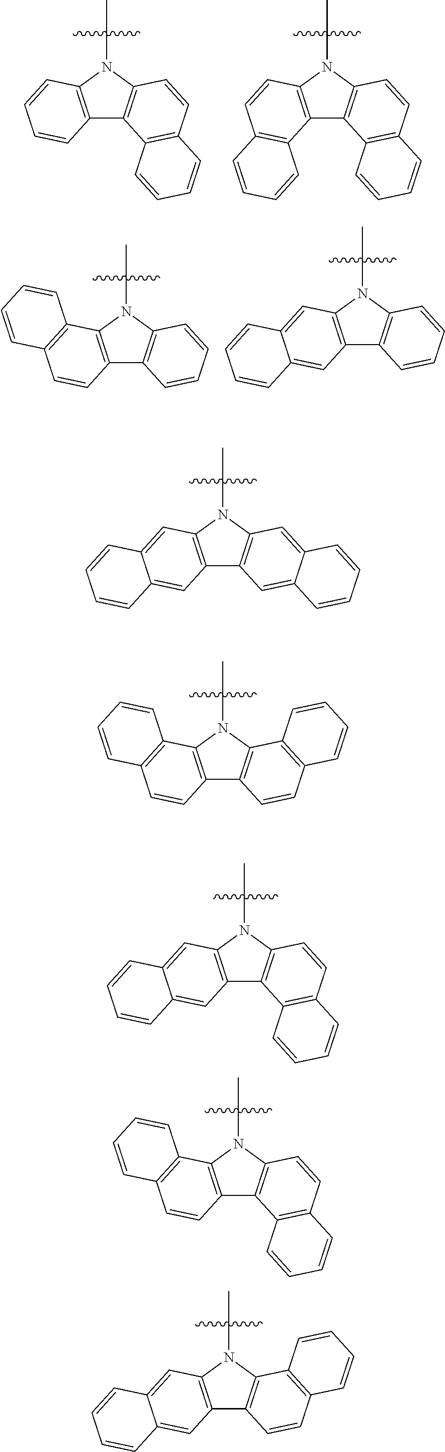

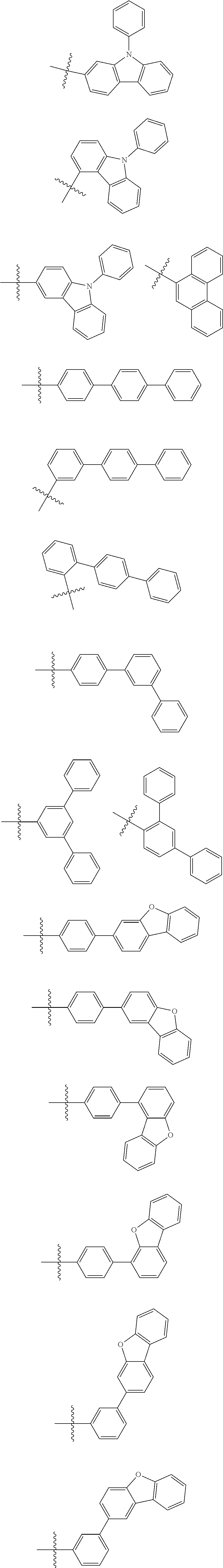

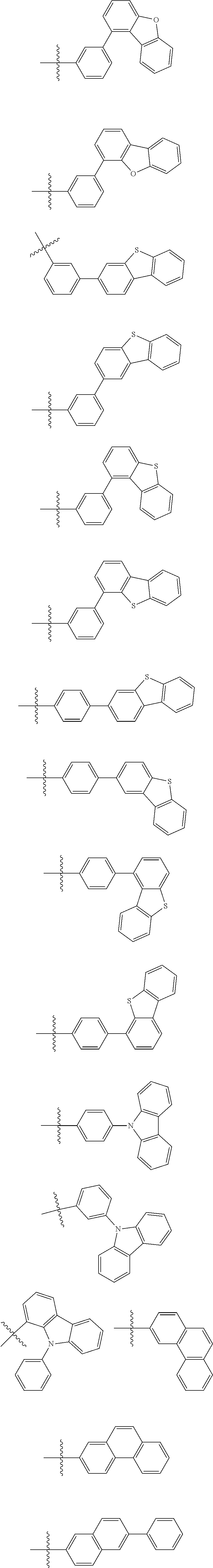

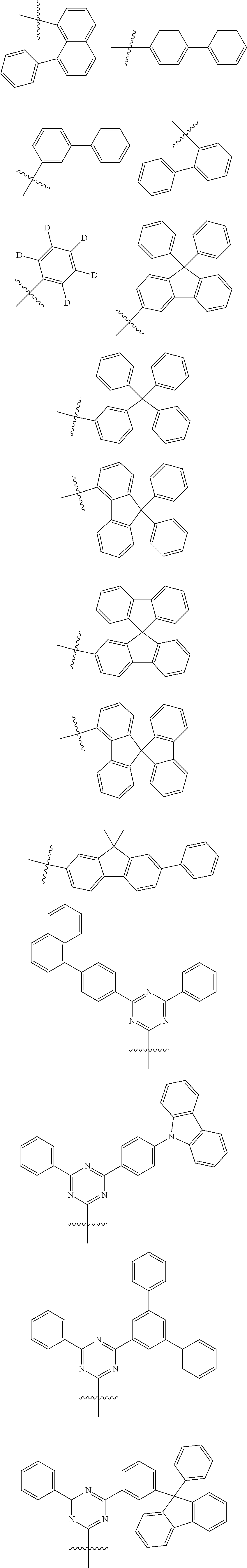

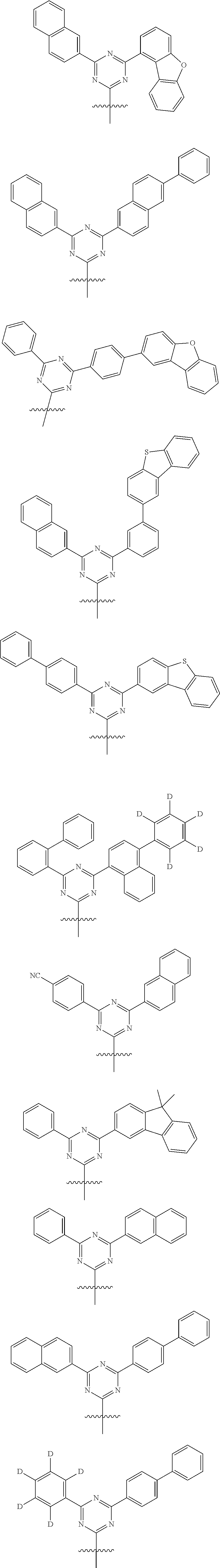

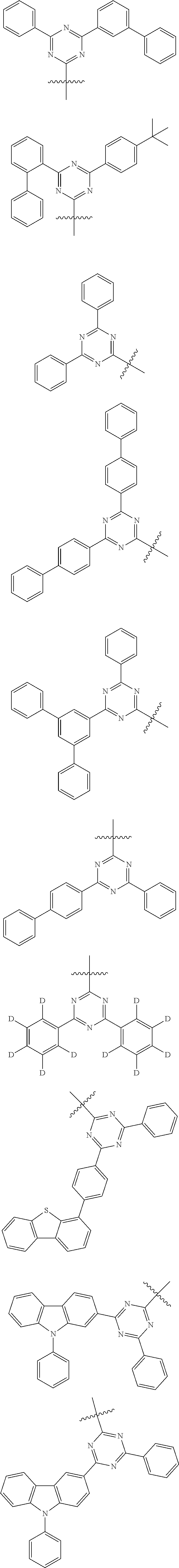

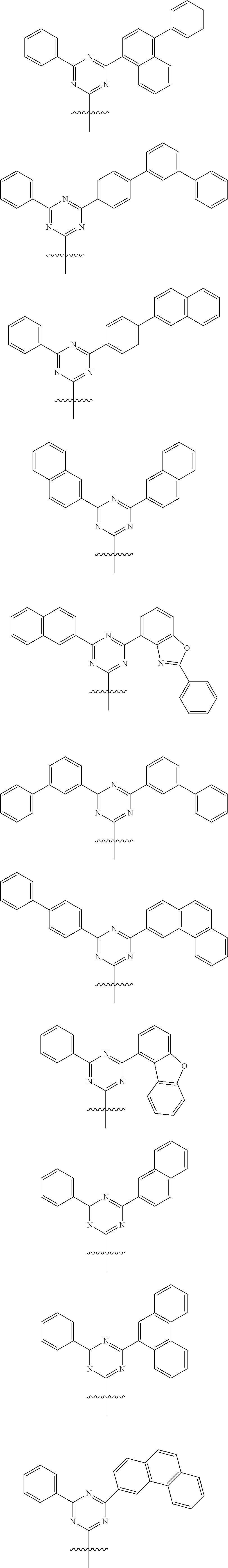

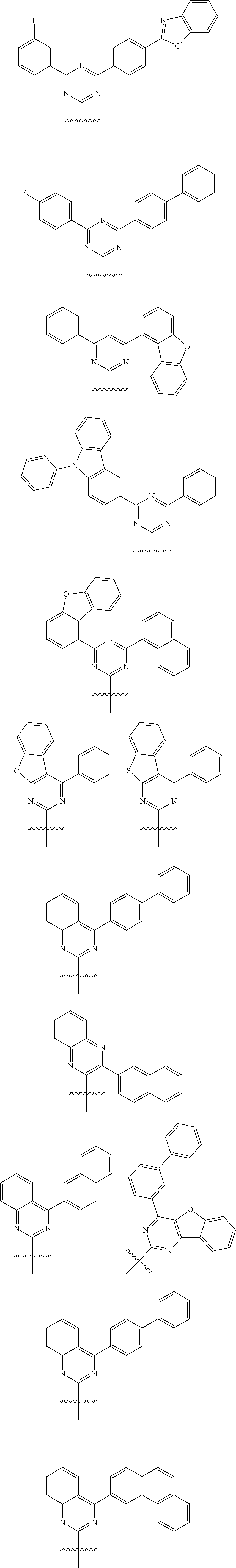

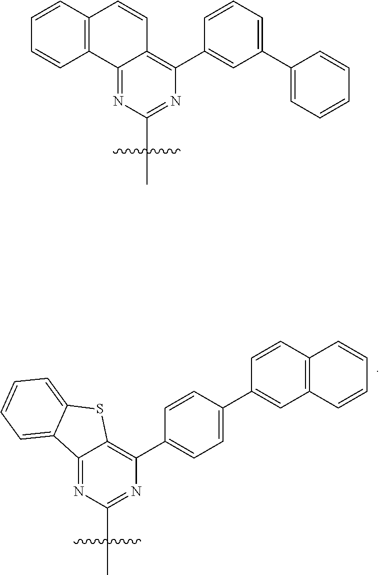 [SMILES: *c1nc(-c2cccc(-c3ccccc3)c2)c2ccc3ccccc3c2n1 ; *c1nc(-c2ccc(-c3ccc4ccccc4c3)cc2)c2sc3ccccc3c2n1]

14. The nitrogen-containing compound according to claim 1, wherein the nitrogen-containing compound is selected from the group consisting of the following compounds:

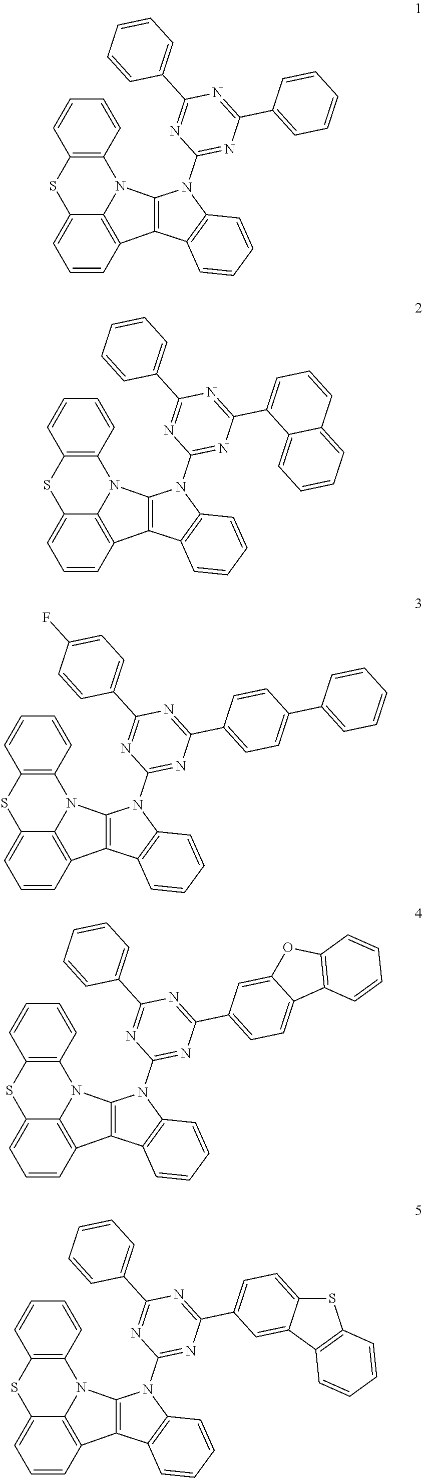

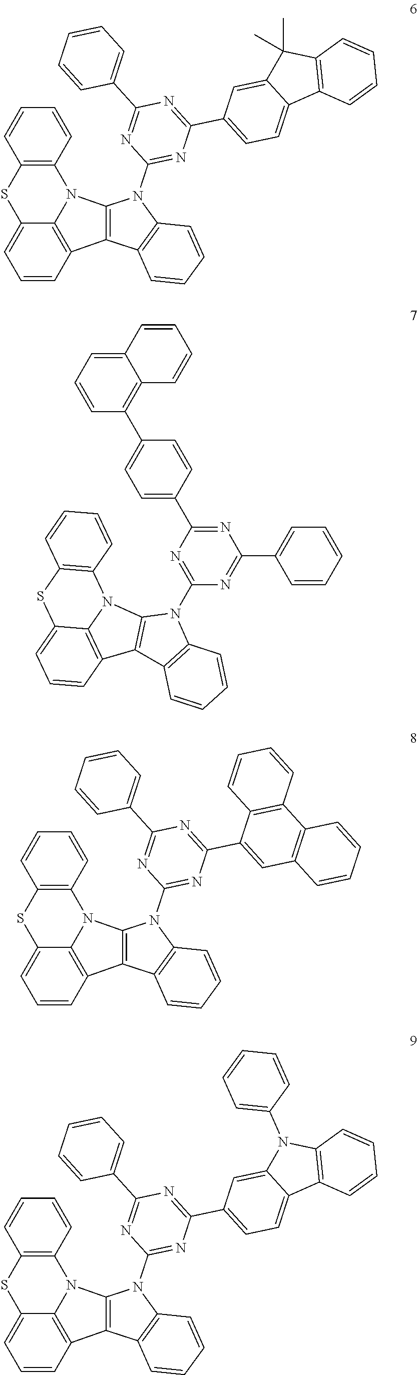

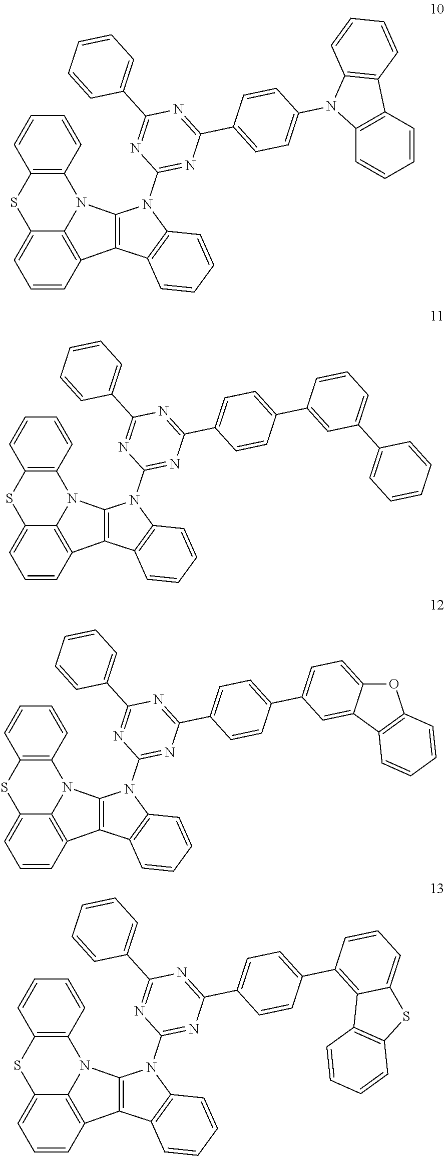

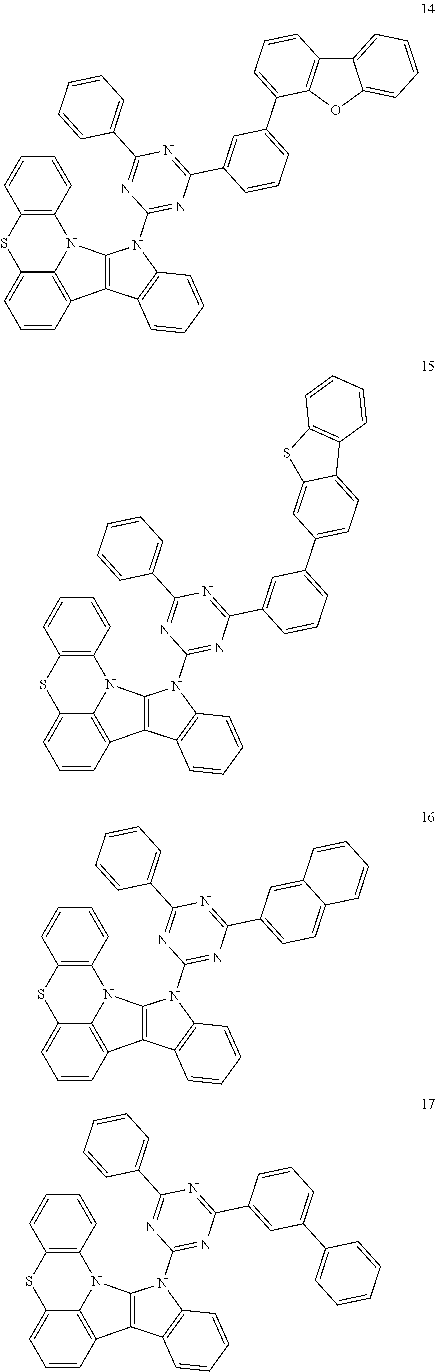

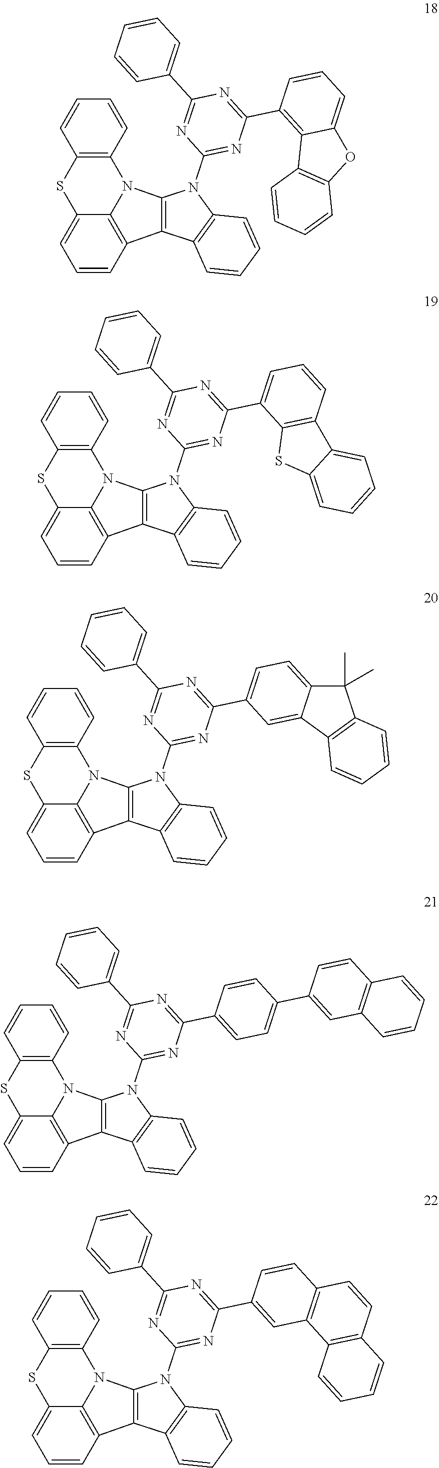

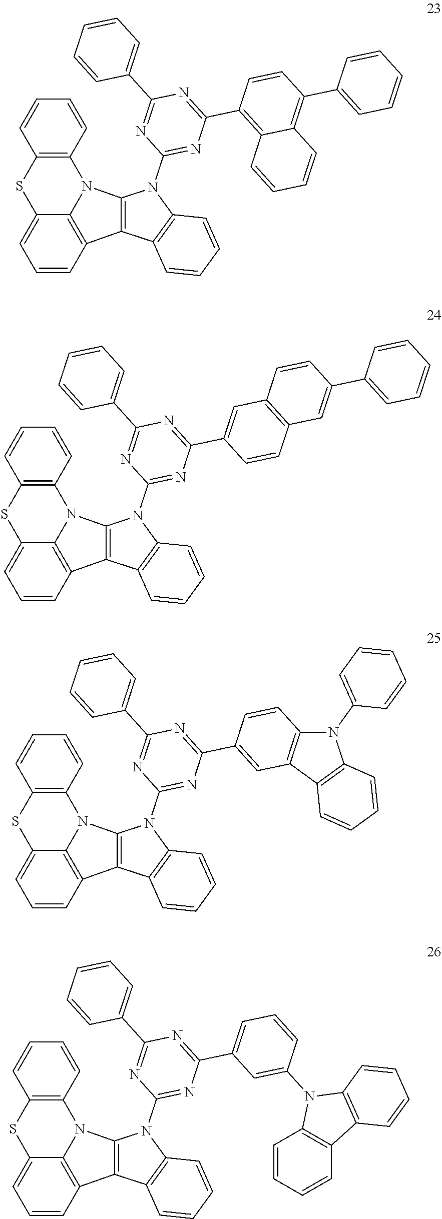 [SMILES: c1ccc(-c2nc(-c3ccc(-c4ccccc4)c4ccccc34)nc(-n3c4ccccc4c4c5cccc6c5n(c43)-c3ccccc3S6)n2)cc1 ; c1ccc(-c2ccc3cc(-c4nc(-c5ccccc5)nc(-n5c6ccccc6c6c7cccc8c7n(c65)-c5ccccc5S8)n4)ccc3c2)cc1 ; c1ccc(-c2nc(-c3ccc4c(c3)c3ccccc3n4-c3ccccc3)nc(-n3c4ccccc4c4c5cccc6c5n(c43)-c3ccccc3S6)n2)cc1 ; c1ccc(-c2nc(-c3cccc(-n4c5ccccc5c5ccccc54)c3)nc(-n3c4ccccc4c4c5cccc6c5n(c43)-c3ccccc3S6)n2)cc1]

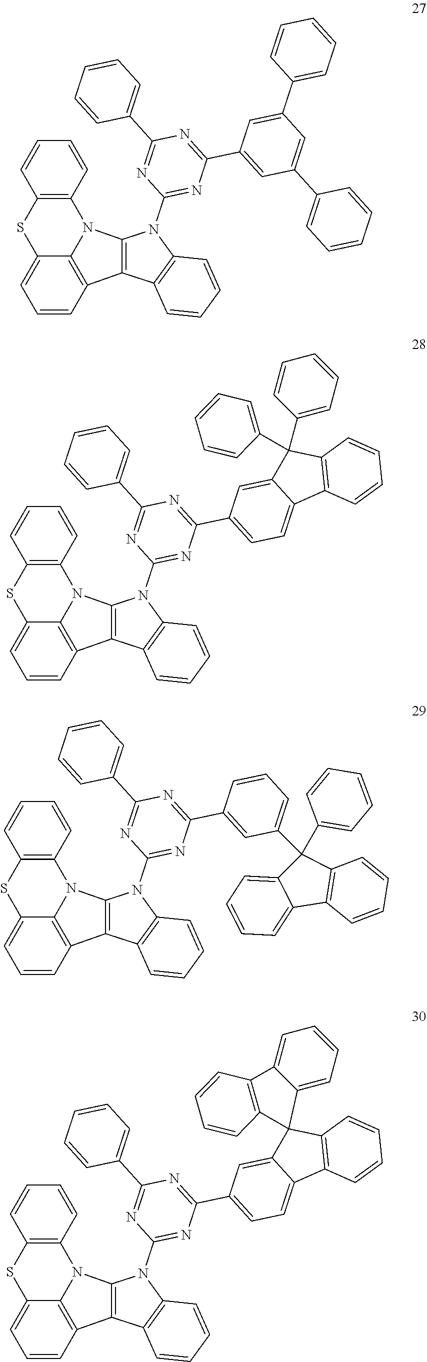 [SMILES: c1ccc(-c2cc(-c3ccccc3)cc(-c3nc(-c4ccccc4)nc(-n4c5ccccc5c5c6cccc7c6n(c54)-c4ccccc4S7)n3)c2)cc1 ; c1ccc(-c2nc(-c3ccc4c(c3)C(c3ccccc3)(c3ccccc3)c3ccccc3-4)nc(-n3c4ccccc4c4c5cccc6c5n(c43)-c3ccccc3S6)n2)cc1 ; c1ccc(-c2nc(-c3cccc(C4(c5ccccc5)c5ccccc5-c5ccccc54)c3)nc(-n3c4ccccc4c4c5cccc6c5n(c43)-c3ccccc3S6)n2)cc1 ; c1ccc(-c2nc(-c3ccc4c(c3)C3(c5ccccc5-c5ccccc53)c3ccccc3-4)nc(-n3c4ccccc4c4c5cccc6c5n(c43)-c3ccccc3S6)n2)cc1]

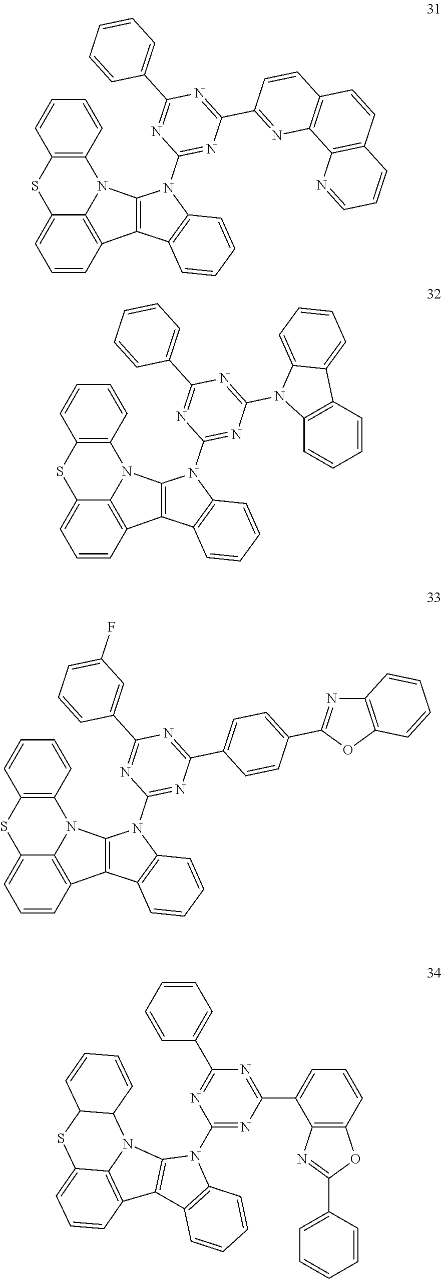

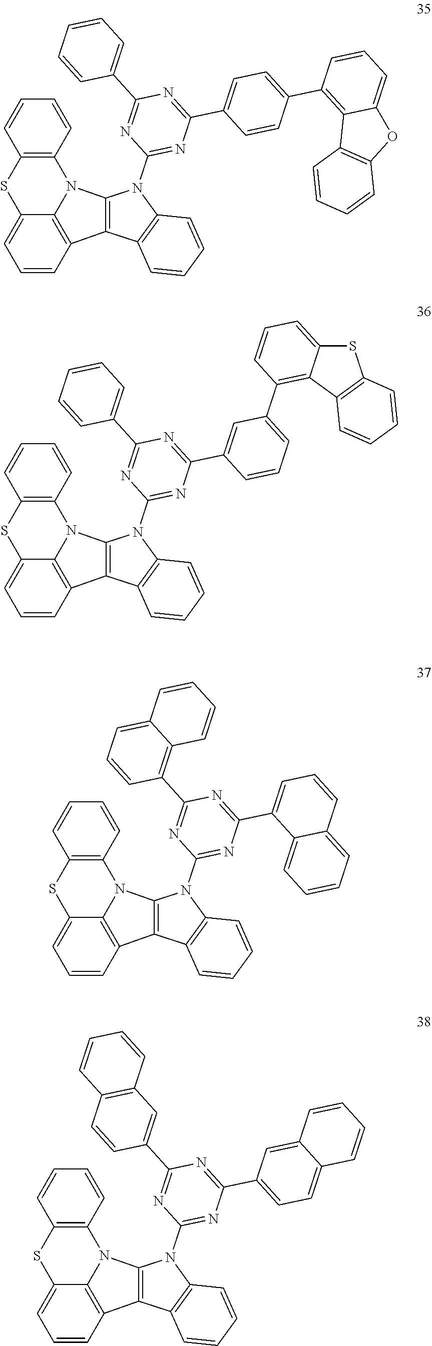 [SMILES: c1ccc(-c2nc(-c3ccc(-c4cccc5oc6ccccc6c45)cc3)nc(-n3c4ccccc4c4c5cccc6c5n(c43)-c3ccccc3S6)n2)cc1 ; c1ccc(-c2nc(-c3cccc(-c4cccc5sc6ccccc6c45)c3)nc(-n3c4ccccc4c4c5cccc6c5n(c43)-c3ccccc3S6)n2)cc1 ; c1ccc2c(c1)Sc1cccc3c4c5ccccc5n(-c5nc(-c6cccc7ccccc67)nc(-c6cccc7ccccc67)n5)c4n-2c13 ; c1ccc2c(c1)Sc1cccc3c4c5ccccc5n(-c5nc(-c6ccc7ccccc7c6)nc(-c6ccc7ccccc7c6)n5)c4n-2c13]

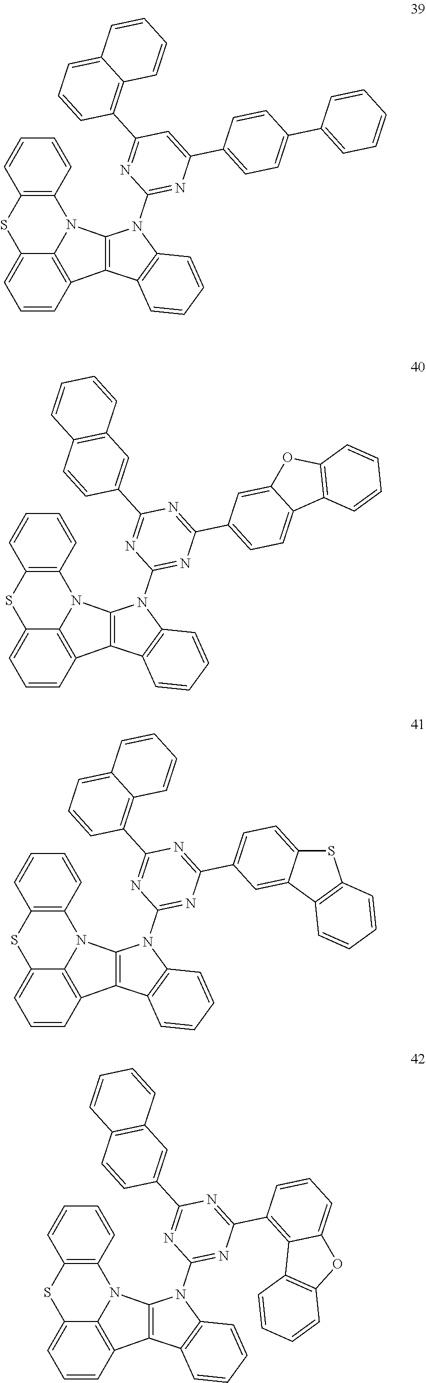 [SMILES: c1ccc(-c2ccc(-c3cc(-c4cccc5ccccc45)nc(-n4c5ccccc5c5c6cccc7c6n(c54)-c4ccccc4S7)n3)cc2)cc1 ; c1ccc2c(c1)Sc1cccc3c4c5ccccc5n(-c5nc(-c6ccc7ccccc7c6)nc(-c6ccc7c(c6)oc6ccccc67)n5)c4n-2c13 ; c1ccc2c(c1)Sc1cccc3c4c5ccccc5n(-c5nc(-c6ccc7sc8ccccc8c7c6)nc(-c6cccc7ccccc67)n5)c4n-2c13 ; c1ccc2c(c1)Sc1cccc3c4c5ccccc5n(-c5nc(-c6ccc7ccccc7c6)nc(-c6cccc7oc8ccccc8c67)n5)c4n-2c13]

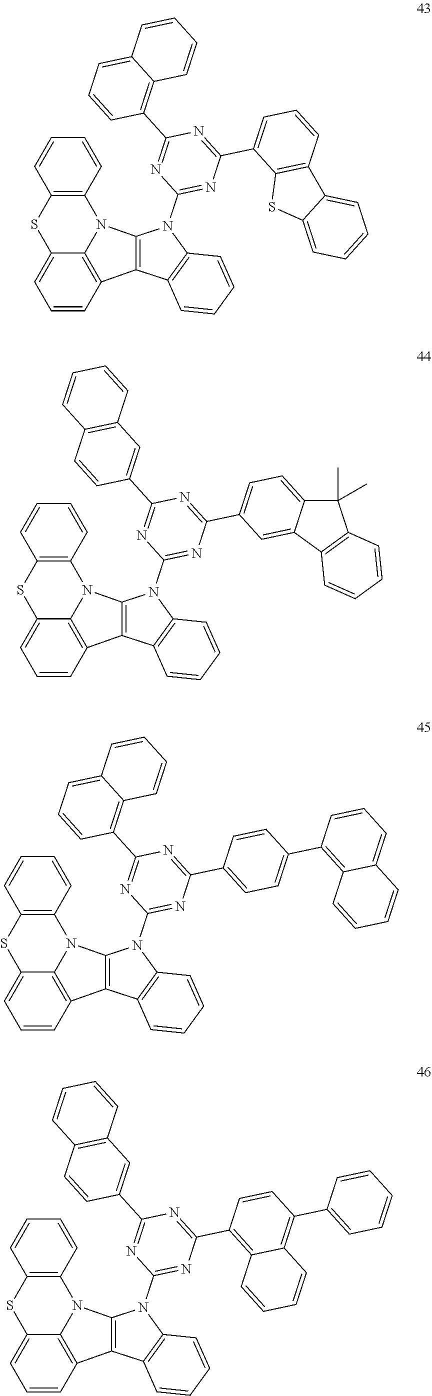 [SMILES: c1ccc2c(c1)Sc1cccc3c4c5ccccc5n(-c5nc(-c6cccc7ccccc67)nc(-c6cccc7c6sc6ccccc67)n5)c4n-2c13 ; CC1(C)c2ccccc2-c2cc(-c3nc(-c4ccc5ccccc5c4)nc(-n4c5ccccc5c5c6cccc7c6n(c54)-c4ccccc4S7)n3)ccc21 ; c1ccc2c(c1)Sc1cccc3c4c5ccccc5n(-c5nc(-c6ccc(-c7cccc8ccccc78)cc6)nc(-c6cccc7ccccc67)n5)c4n-2c13 ; c1ccc(-c2ccc(-c3nc(-c4ccc5ccccc5c4)nc(-n4c5ccccc5c5c6cccc7c6n(c54)-c4ccccc4S7)n3)c3ccccc23)cc1]

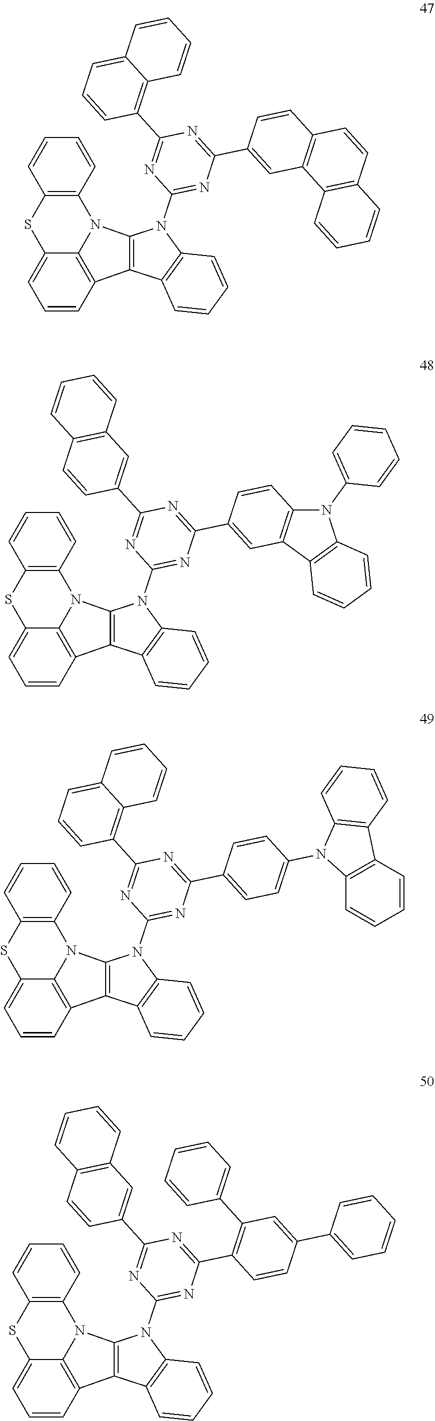 [SMILES: c1ccc2c(c1)Sc1cccc3c4c5ccccc5n(-c5nc(-c6ccc7ccc8ccccc8c7c6)nc(-c6cccc7ccccc67)n5)c4n-2c13 ; c1ccc(-n2c3ccccc3c3cc(-c4nc(-c5ccc6ccccc6c5)nc(-n5c6ccccc6c6c7cccc8c7n(c65)-c5ccccc5S8)n4)ccc32)cc1 ; c1ccc2c(c1)Sc1cccc3c4c5ccccc5n(-c5nc(-c6ccc(-n7c8ccccc8c8ccccc87)cc6)nc(-c6cccc7ccccc67)n5)c4n-2c13 ; c1ccc(-c2ccc(-c3nc(-c4ccc5ccccc5c4)nc(-n4c5ccccc5c5c6cccc7c6n(c54)-c4ccccc4S7)n3)c(-c3ccccc3)c2)cc1]

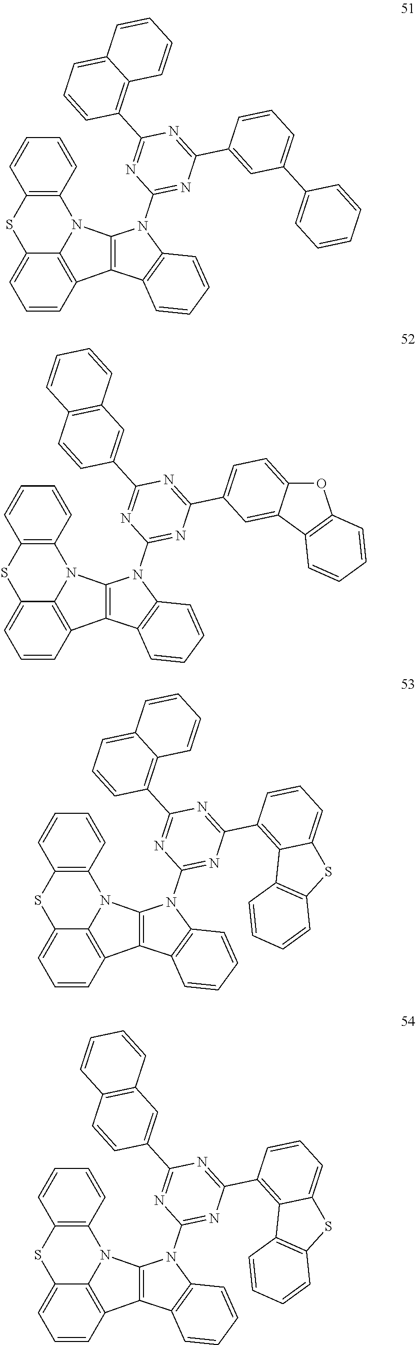

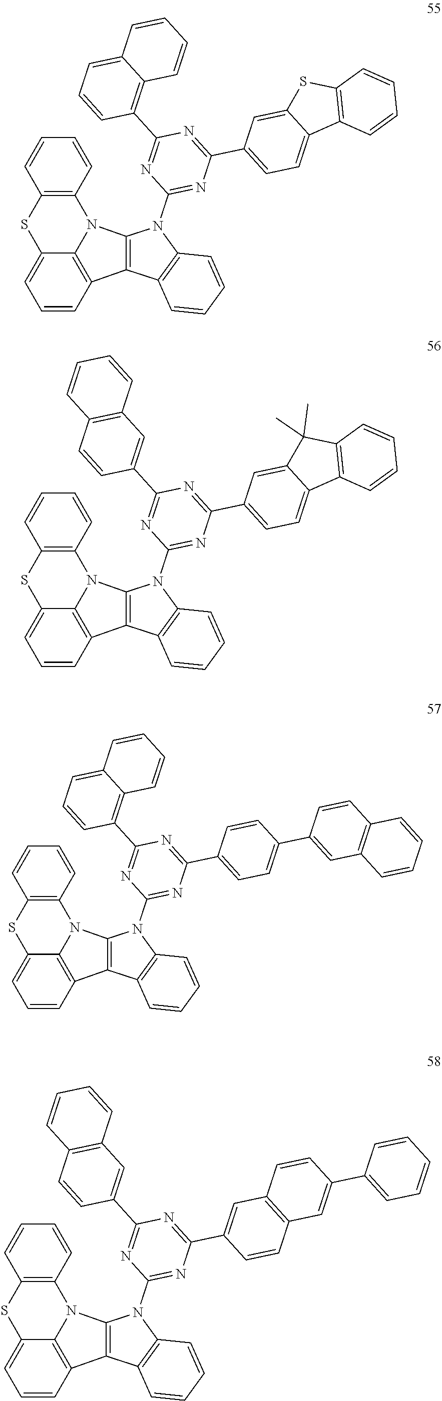 [SMILES: c1ccc2c(c1)Sc1cccc3c4c5ccccc5n(-c5nc(-c6ccc7c(c6)sc6ccccc67)nc(-c6cccc7ccccc67)n5)c4n-2c13 ; CC1(C)c2ccccc2-c2ccc(-c3nc(-c4ccc5ccccc5c4)nc(-n4c5ccccc5c5c6cccc7c6n(c54)-c4ccccc4S7)n3)cc21 ; c1ccc2c(c1)Sc1cccc3c4c5ccccc5n(-c5nc(-c6ccc(-c7ccc8ccccc8c7)cc6)nc(-c6cccc7ccccc67)n5)c4n-2c13 ; c1ccc(-c2ccc3cc(-c4nc(-c5ccc6ccccc6c5)nc(-n5c6ccccc6c6c7cccc8c7n(c65)-c5ccccc5S8)n4)ccc3c2)cc1]

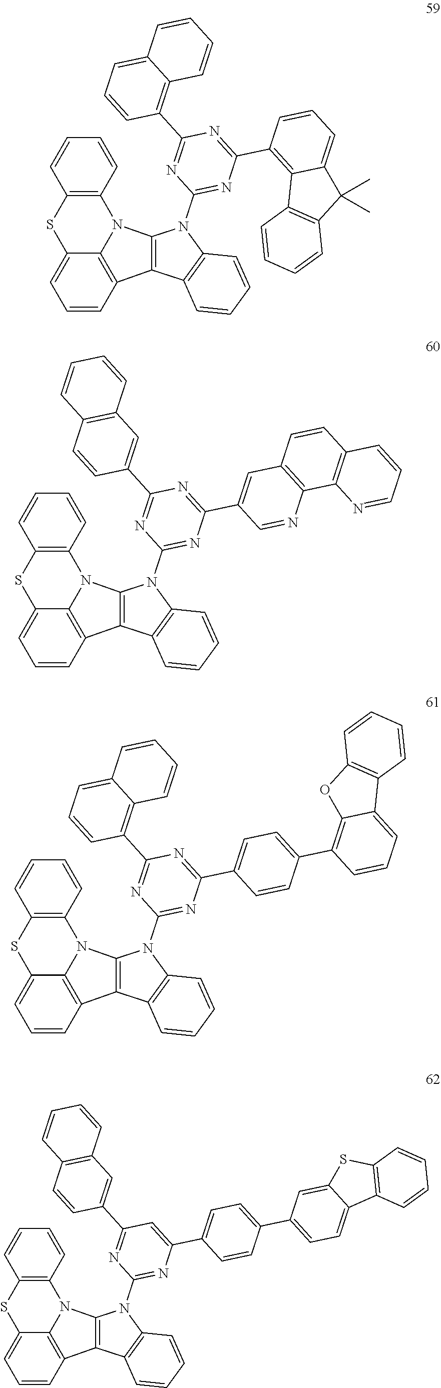

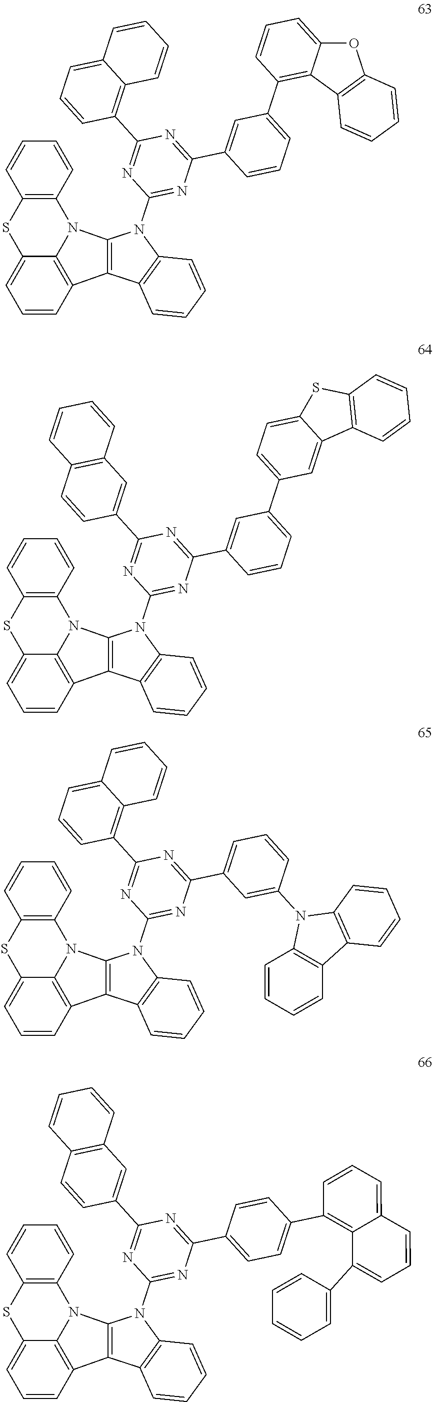

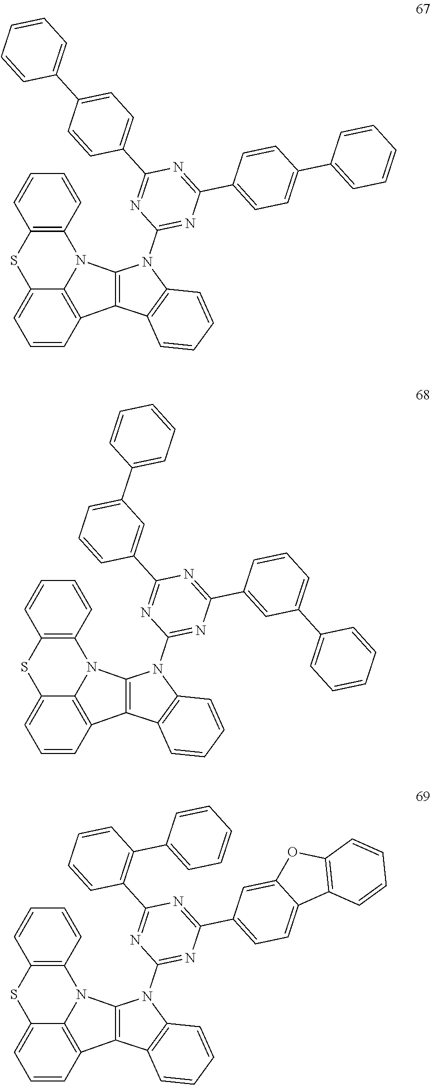 [SMILES: c1ccc(-c2ccc(-c3nc(-c4ccc(-c5ccccc5)cc4)nc(-n4c5ccccc5c5c6cccc7c6n(c54)-c4ccccc4S7)n3)cc2)cc1 ; c1ccc(-c2cccc(-c3nc(-c4cccc(-c5ccccc5)c4)nc(-n4c5ccccc5c5c6cccc7c6n(c54)-c4ccccc4S7)n3)c2)cc1 ; c1ccc(-c2ccccc2-c2nc(-c3ccc4c(c3)oc3ccccc34)nc(-n3c4ccccc4c4c5cccc6c5n(c43)-c3ccccc3S6)n2)cc1]

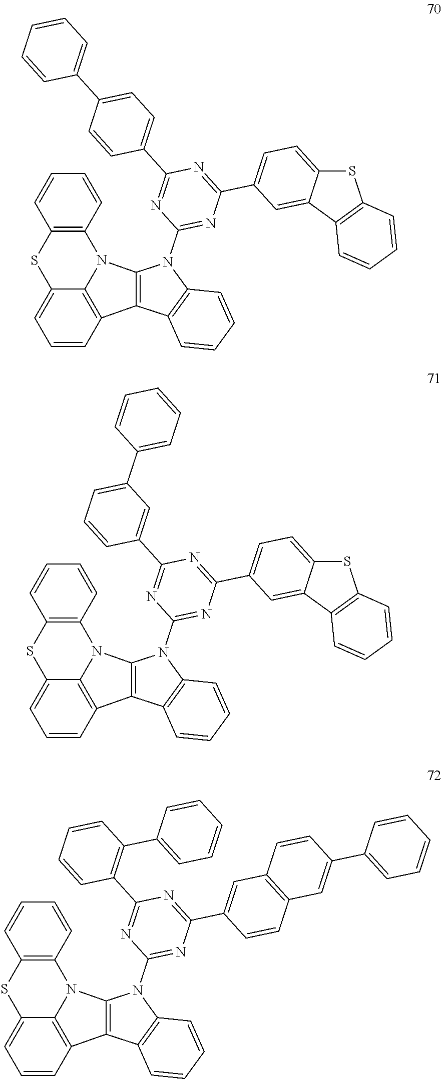 [SMILES: c1ccc(-c2ccc(-c3nc(-c4ccc5sc6ccccc6c5c4)nc(-n4c5ccccc5c5c6cccc7c6n(c54)-c4ccccc4S7)n3)cc2)cc1 ; c1ccc(-c2cccc(-c3nc(-c4ccc5sc6ccccc6c5c4)nc(-n4c5ccccc5c5c6cccc7c6n(c54)-c4ccccc4S7)n3)c2)cc1 ; c1ccc(-c2ccc3cc(-c4nc(-c5ccccc5-c5ccccc5)nc(-n5c6ccccc6c6c7cccc8c7n(c65)-c5ccccc5S8)n4)ccc3c2)cc1]

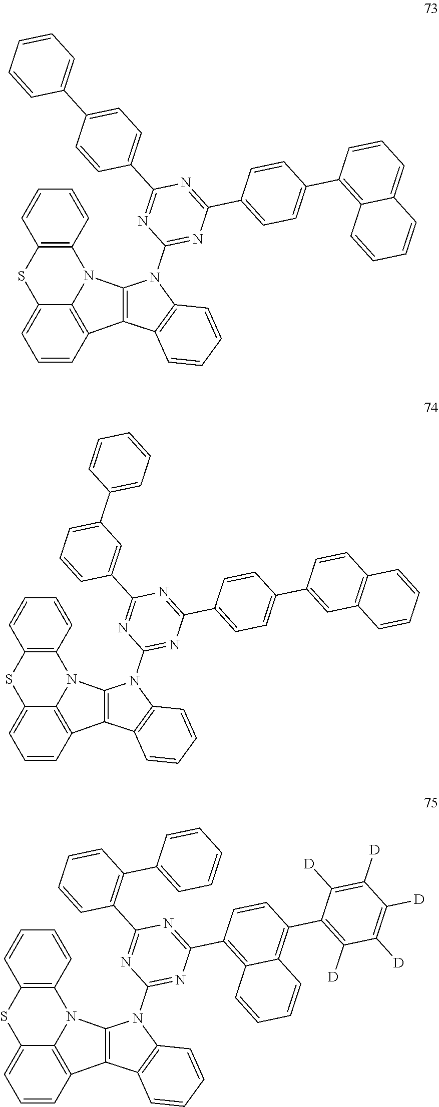 [SMILES: c1ccc(-c2ccc(-c3nc(-c4ccc(-c5cccc6ccccc56)cc4)nc(-n4c5ccccc5c5c6cccc7c6n(c54)-c4ccccc4S7)n3)cc2)cc1 ; c1ccc(-c2cccc(-c3nc(-c4ccc(-c5ccc6ccccc6c5)cc4)nc(-n4c5ccccc5c5c6cccc7c6n(c54)-c4ccccc4S7)n3)c2)cc1 ; [2H]c1c([2H])c([2H])c(-c2ccc(-c3nc(-c4ccccc4-c4ccccc4)nc(-n4c5ccccc5c5c6cccc7c6n(c54)-c4ccccc4S7)n3)c3ccccc23)c([2H])c1[2H]]

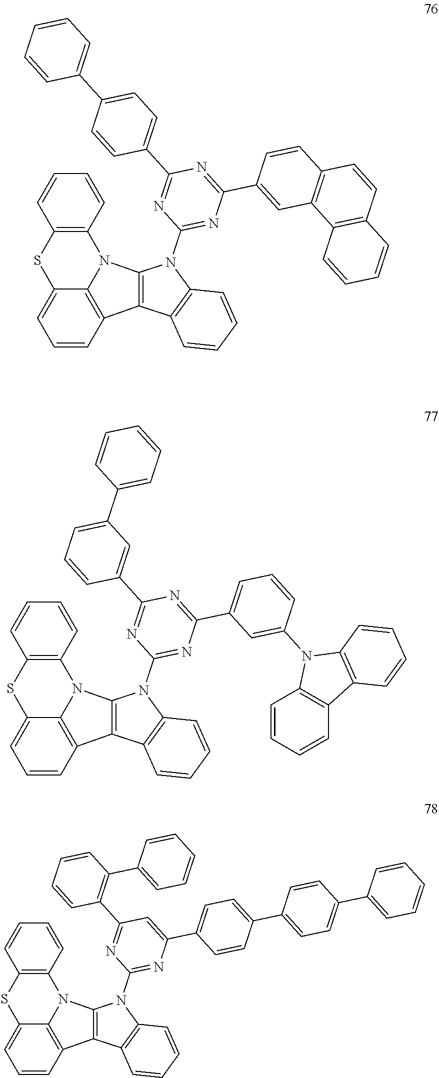 [SMILES: c1ccc(-c2ccc(-c3nc(-c4ccc5ccc6ccccc6c5c4)nc(-n4c5ccccc5c5c6cccc7c6n(c54)-c4ccccc4S7)n3)cc2)cc1 ; c1ccc(-c2cccc(-c3nc(-c4cccc(-n5c6ccccc6c6ccccc65)c4)nc(-n4c5ccccc5c5c6cccc7c6n(c54)-c4ccccc4S7)n3)c2)cc1 ; c1ccc(-c2ccc(-c3ccc(-c4cc(-c5ccccc5-c5ccccc5)nc(-n5c6ccccc6c6c7cccc8c7n(c65)-c5ccccc5S8)n4)cc3)cc2)cc1]

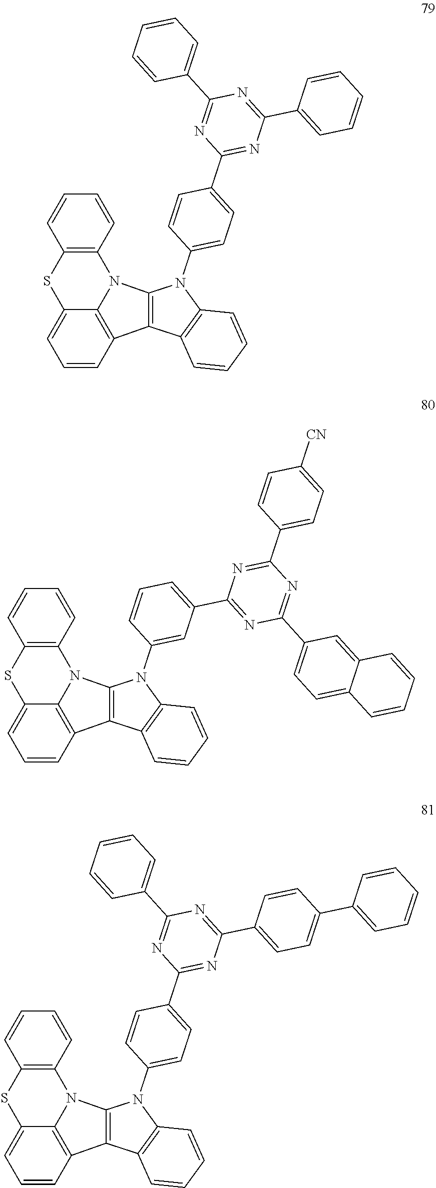 [SMILES: c1ccc(-c2nc(-c3ccccc3)nc(-c3ccc(-n4c5ccccc5c5c6cccc7c6n(c54)-c4ccccc4S7)cc3)n2)cc1 ; N#Cc1ccc(-c2nc(-c3cccc(-n4c5ccccc5c5c6cccc7c6n(c54)-c4ccccc4S7)c3)nc(-c3ccc4ccccc4c3)n2)cc1 ; c1ccc(-c2ccc(-c3nc(-c4ccccc4)nc(-c4ccc(-n5c6ccccc6c6c7cccc8c7n(c65)-c5ccccc5S8)cc4)n3)cc2)cc1]

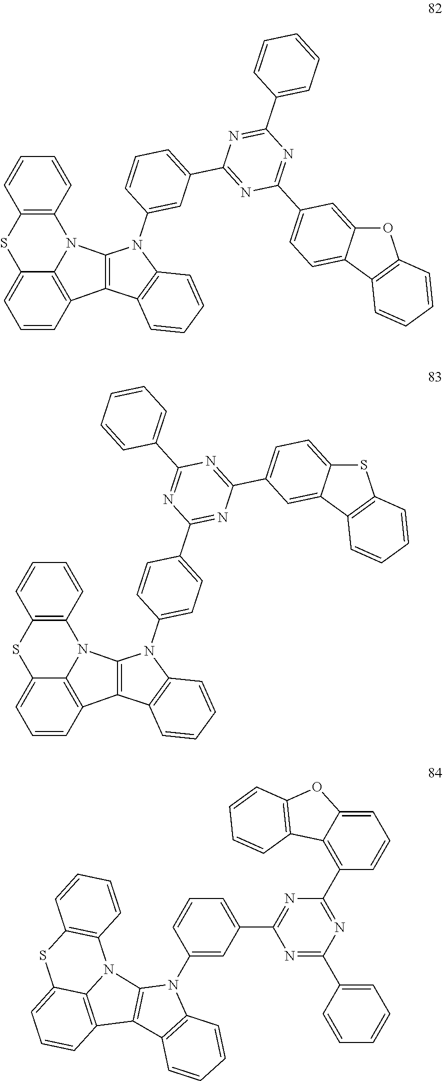 [SMILES: c1ccc(-c2nc(-c3cccc(-n4c5ccccc5c5c6cccc7c6n(c54)-c4ccccc4S7)c3)nc(-c3ccc4c(c3)oc3ccccc34)n2)cc1 ; c1ccc(-c2nc(-c3ccc(-n4c5ccccc5c5c6cccc7c6n(c54)-c4ccccc4S7)cc3)nc(-c3ccc4sc5ccccc5c4c3)n2)cc1 ; c1ccc(-c2nc(-c3cccc(-n4c5ccccc5c5c6cccc7c6n(c54)-c4ccccc4S7)c3)nc(-c3cccc4oc5ccccc5c34)n2)cc1]

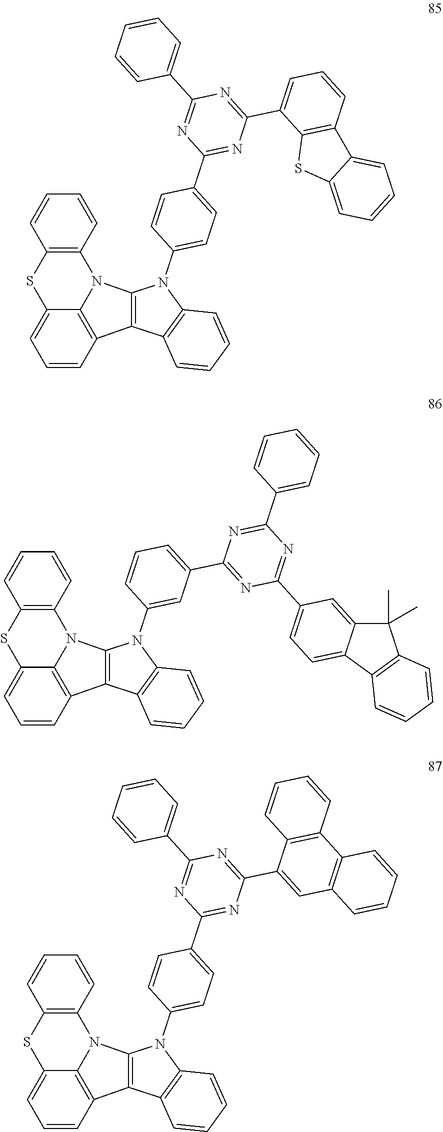 [SMILES: c1ccc(-c2nc(-c3ccc(-n4c5ccccc5c5c6cccc7c6n(c54)-c4ccccc4S7)cc3)nc(-c3cccc4c3sc3ccccc34)n2)cc1 ; CC1(C)c2ccccc2-c2ccc(-c3nc(-c4ccccc4)nc(-c4cccc(-n5c6ccccc6c6c7cccc8c7n(c65)-c5ccccc5S8)c4)n3)cc21 ; c1ccc(-c2nc(-c3ccc(-n4c5ccccc5c5c6cccc7c6n(c54)-c4ccccc4S7)cc3)nc(-c3cc4ccccc4c4ccccc34)n2)cc1]

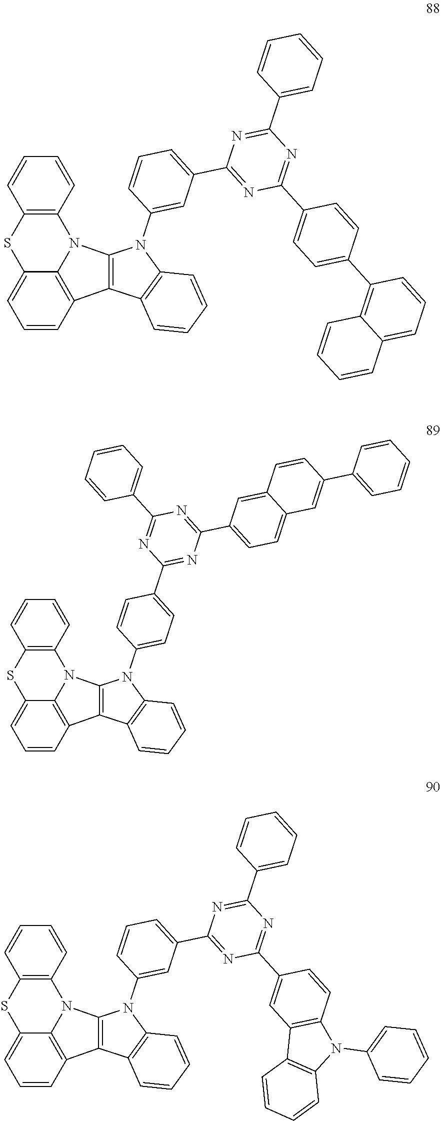 [SMILES: c1ccc(-c2nc(-c3ccc(-c4cccc5ccccc45)cc3)nc(-c3cccc(-n4c5ccccc5c5c6cccc7c6n(c54)-c4ccccc4S7)c3)n2)cc1 ; c1ccc(-c2ccc3cc(-c4nc(-c5ccccc5)nc(-c5ccc(-n6c7ccccc7c7c8cccc9c8n(c76)-c6ccccc6S9)cc5)n4)ccc3c2)cc1 ; c1ccc(-c2nc(-c3cccc(-n4c5ccccc5c5c6cccc7c6n(c54)-c4ccccc4S7)c3)nc(-c3ccc4c(c3)c3ccccc3n4-c3ccccc3)n2)cc1]

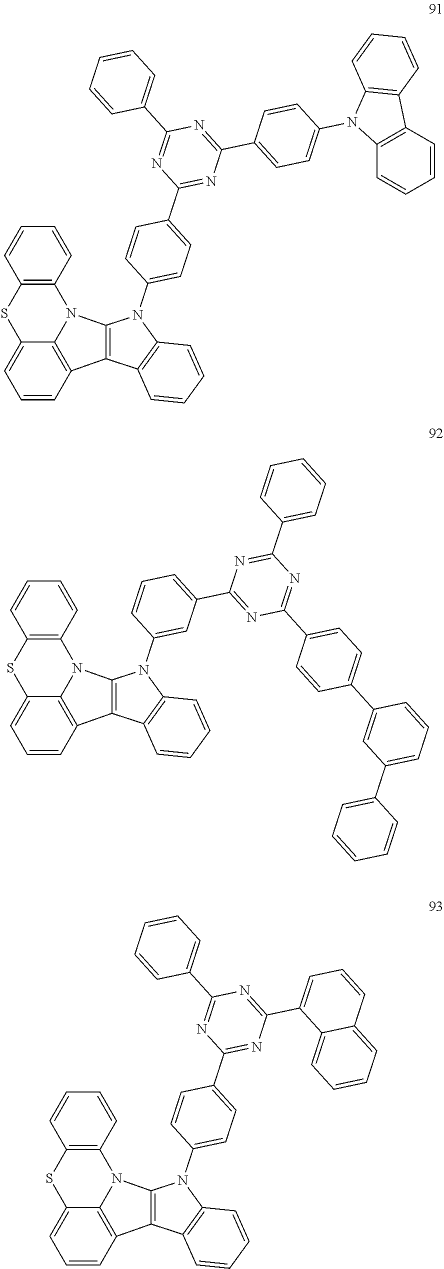 [SMILES: c1ccc(-c2nc(-c3ccc(-n4c5ccccc5c5ccccc54)cc3)nc(-c3ccc(-n4c5ccccc5c5c6cccc7c6n(c54)-c4ccccc4S7)cc3)n2)cc1 ; c1ccc(-c2cccc(-c3ccc(-c4nc(-c5ccccc5)nc(-c5cccc(-n6c7ccccc7c7c8cccc9c8n(c76)-c6ccccc6S9)c5)n4)cc3)c2)cc1 ; c1ccc(-c2nc(-c3ccc(-n4c5ccccc5c5c6cccc7c6n(c54)-c4ccccc4S7)cc3)nc(-c3cccc4ccccc34)n2)cc1]

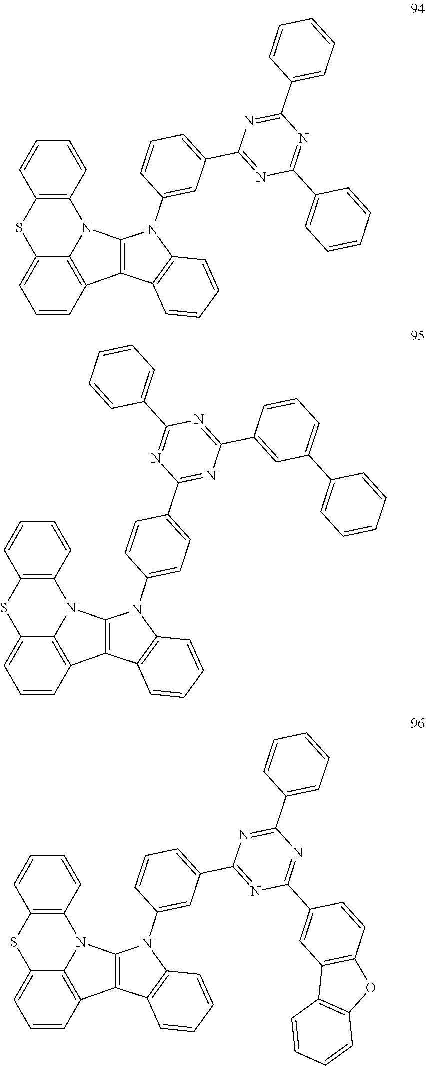

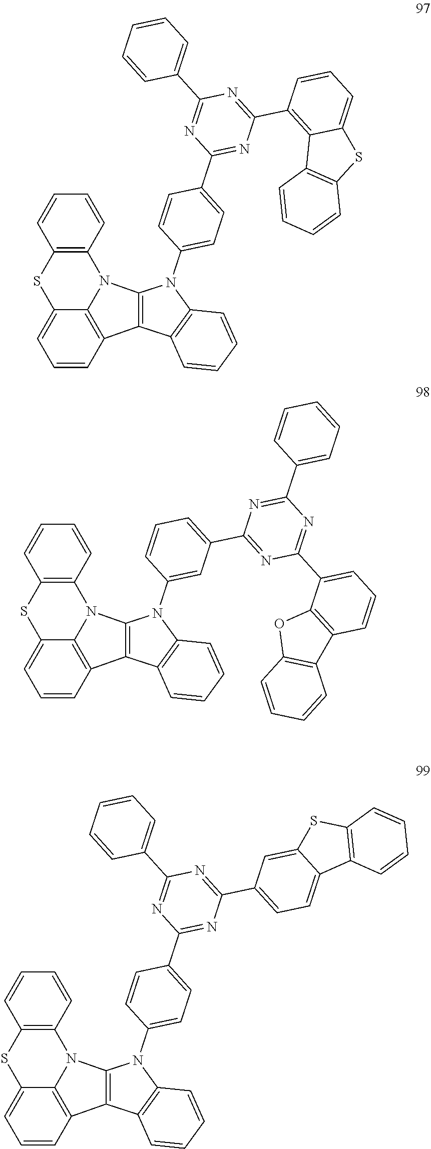 [SMILES: c1ccc(-c2nc(-c3ccc(-n4c5ccccc5c5c6cccc7c6n(c54)-c4ccccc4S7)cc3)nc(-c3cccc4sc5ccccc5c34)n2)cc1 ; c1ccc(-c2nc(-c3cccc(-n4c5ccccc5c5c6cccc7c6n(c54)-c4ccccc4S7)c3)nc(-c3cccc4c3oc3ccccc34)n2)cc1 ; c1ccc(-c2nc(-c3ccc(-n4c5ccccc5c5c6cccc7c6n(c54)-c4ccccc4S7)cc3)nc(-c3ccc4c(c3)sc3ccccc34)n2)cc1]

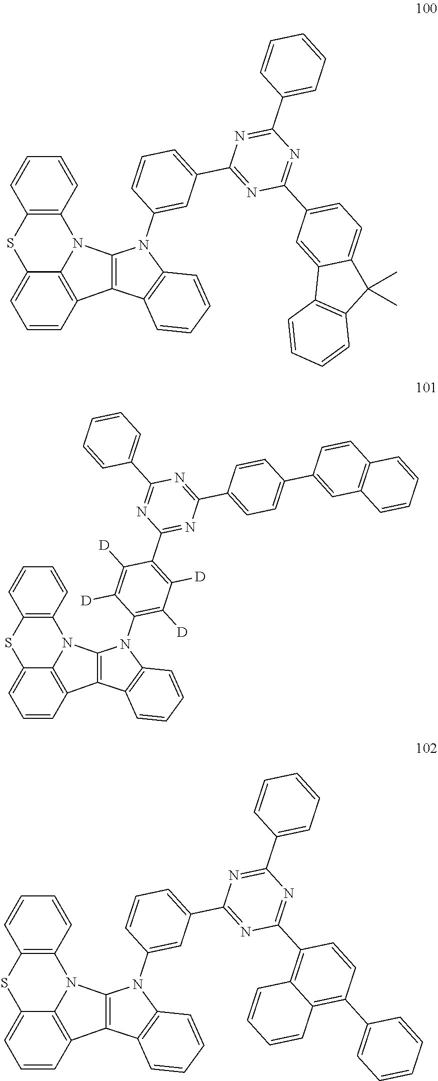 [SMILES: CC1(C)c2ccccc2-c2cc(-c3nc(-c4ccccc4)nc(-c4cccc(-n5c6ccccc6c6c7cccc8c7n(c65)-c5ccccc5S8)c4)n3)ccc21 ; [2H]c1c([2H])c(-n2c3ccccc3c3c4cccc5c4n(c32)-c2ccccc2S5)c([2H])c([2H])c1-c1nc(-c2ccccc2)nc(-c2ccc(-c3ccc4ccccc4c3)cc2)n1 ; c1ccc(-c2nc(-c3cccc(-n4c5ccccc5c5c6cccc7c6n(c54)-c4ccccc4S7)c3)nc(-c3ccc(-c4ccccc4)c4ccccc34)n2)cc1]

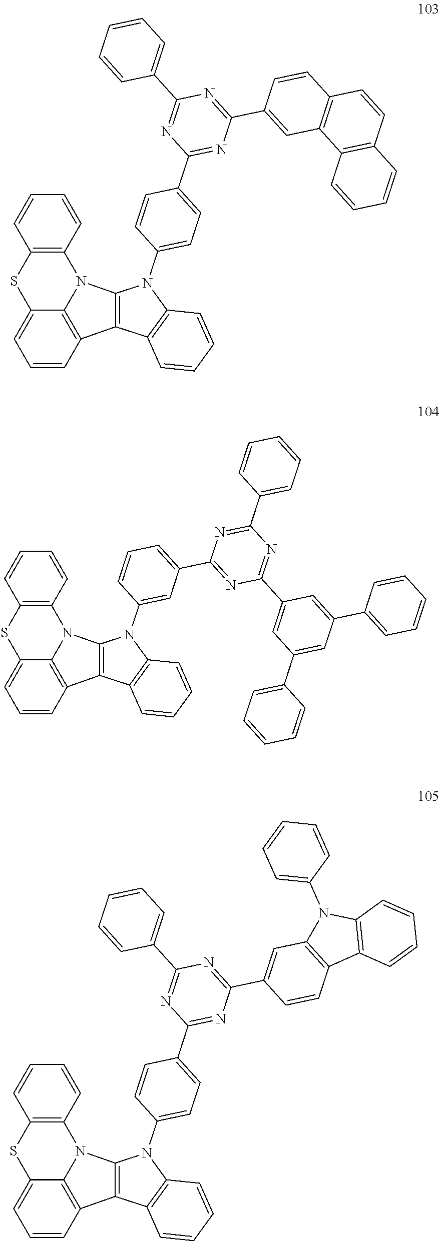 [SMILES: c1ccc(-c2nc(-c3ccc(-n4c5ccccc5c5c6cccc7c6n(c54)-c4ccccc4S7)cc3)nc(-c3ccc4ccc5ccccc5c4c3)n2)cc1 ; c1ccc(-c2cc(-c3ccccc3)cc(-c3nc(-c4ccccc4)nc(-c4cccc(-n5c6ccccc6c6c7cccc8c7n(c65)-c5ccccc5S8)c4)n3)c2)cc1 ; c1ccc(-c2nc(-c3ccc(-n4c5ccccc5c5c6cccc7c6n(c54)-c4ccccc4S7)cc3)nc(-c3ccc4c5ccccc5n(-c5ccccc5)c4c3)n2)cc1]

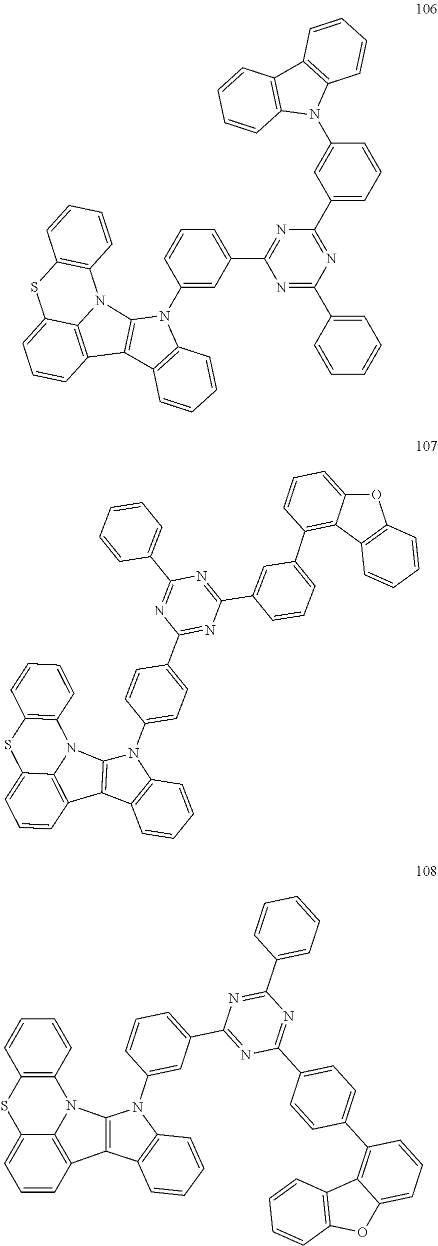 [SMILES: c1ccc(-c2nc(-c3cccc(-n4c5ccccc5c5ccccc54)c3)nc(-c3cccc(-n4c5ccccc5c5c6cccc7c6n(c54)-c4ccccc4S7)c3)n2)cc1 ; c1ccc(-c2nc(-c3ccc(-n4c5ccccc5c5c6cccc7c6n(c54)-c4ccccc4S7)cc3)nc(-c3cccc(-c4cccc5oc6ccccc6c45)c3)n2)cc1 ; c1ccc(-c2nc(-c3ccc(-c4cccc5oc6ccccc6c45)cc3)nc(-c3cccc(-n4c5ccccc5c5c6cccc7c6n(c54)-c4ccccc4S7)c3)n2)cc1]

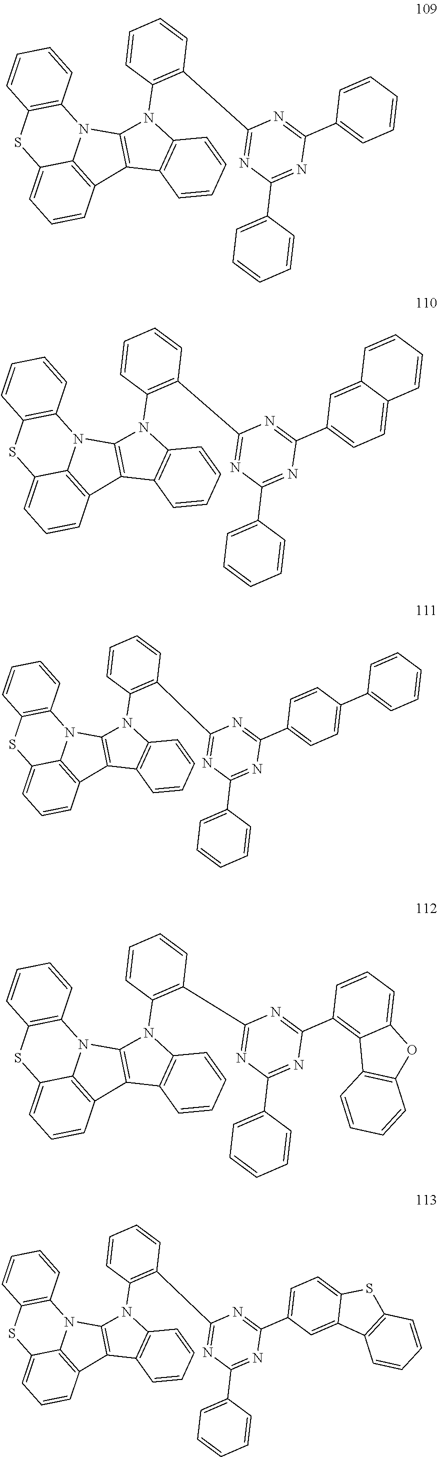 [SMILES: c1ccc(-c2nc(-c3ccccc3)nc(-c3ccccc3-n3c4ccccc4c4c5cccc6c5n(c43)-c3ccccc3S6)n2)cc1 ; c1ccc(-c2nc(-c3ccc4ccccc4c3)nc(-c3ccccc3-n3c4ccccc4c4c5cccc6c5n(c43)-c3ccccc3S6)n2)cc1 ; c1ccc(-c2ccc(-c3nc(-c4ccccc4)nc(-c4ccccc4-n4c5ccccc5c5c6cccc7c6n(c54)-c4ccccc4S7)n3)cc2)cc1 ; c1ccc(-c2nc(-c3ccccc3-n3c4ccccc4c4c5cccc6c5n(c43)-c3ccccc3S6)nc(-c3cccc4oc5ccccc5c34)n2)cc1 ; c1ccc(-c2nc(-c3ccc4sc5ccccc5c4c3)nc(-c3ccccc3-n3c4ccccc4c4c5cccc6c5n(c43)-c3ccccc3S6)n2)cc1]

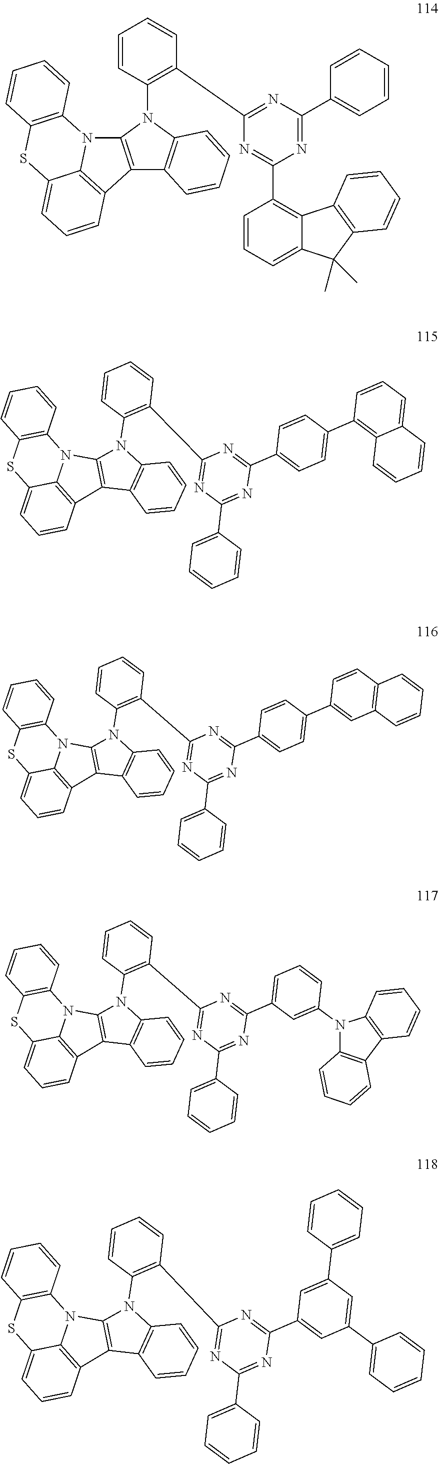

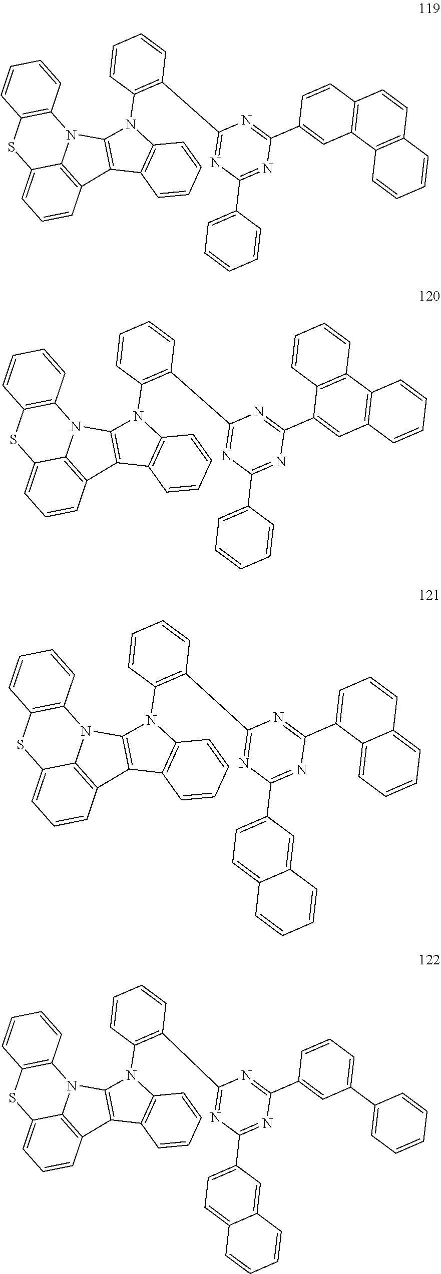 [SMILES: c1ccc(-c2nc(-c3ccc4ccc5ccccc5c4c3)nc(-c3ccccc3-n3c4ccccc4c4c5cccc6c5n(c43)-c3ccccc3S6)n2)cc1 ; c1ccc(-c2nc(-c3ccccc3-n3c4ccccc4c4c5cccc6c5n(c43)-c3ccccc3S6)nc(-c3cc4ccccc4c4ccccc34)n2)cc1 ; c1ccc2c(c1)Sc1cccc3c4c5ccccc5n(-c5ccccc5-c5nc(-c6ccc7ccccc7c6)nc(-c6cccc7ccccc67)n5)c4n-2c13 ; c1ccc(-c2cccc(-c3nc(-c4ccc5ccccc5c4)nc(-c4ccccc4-n4c5ccccc5c5c6cccc7c6n(c54)-c4ccccc4S7)n3)c2)cc1]

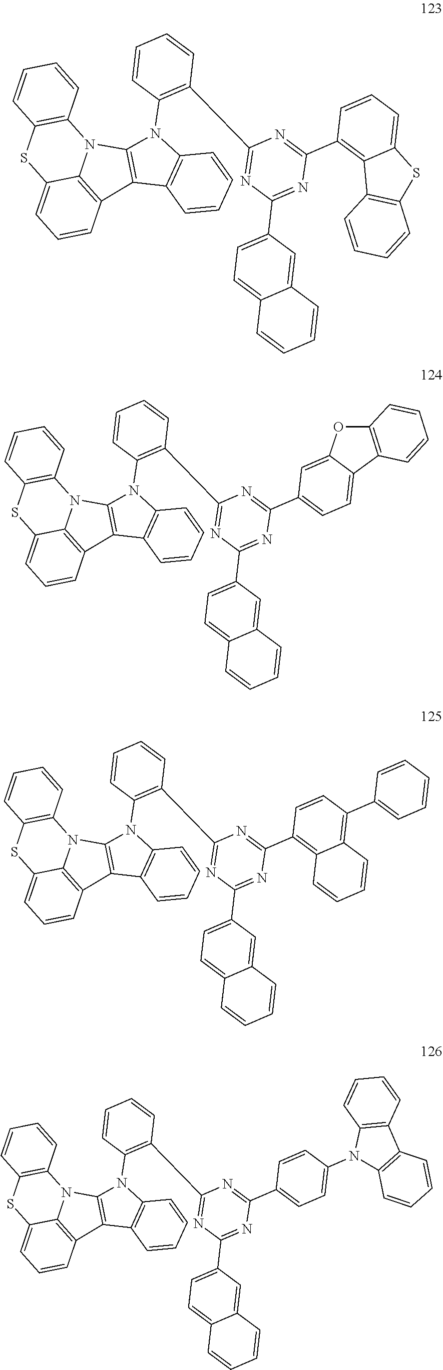 [SMILES: c1ccc2c(c1)Sc1cccc3c4c5ccccc5n(-c5ccccc5-c5nc(-c6ccc7ccccc7c6)nc(-c6cccc7sc8ccccc8c67)n5)c4n-2c13 ; c1ccc2c(c1)Sc1cccc3c4c5ccccc5n(-c5ccccc5-c5nc(-c6ccc7ccccc7c6)nc(-c6ccc7c(c6)oc6ccccc67)n5)c4n-2c13 ; c1ccc(-c2ccc(-c3nc(-c4ccc5ccccc5c4)nc(-c4ccccc4-n4c5ccccc5c5c6cccc7c6n(c54)-c4ccccc4S7)n3)c3ccccc23)cc1 ; c1ccc2c(c1)Sc1cccc3c4c5ccccc5n(-c5ccccc5-c5nc(-c6ccc(-n7c8ccccc8c8ccccc87)cc6)nc(-c6ccc7ccccc7c6)n5)c4n-2c13]

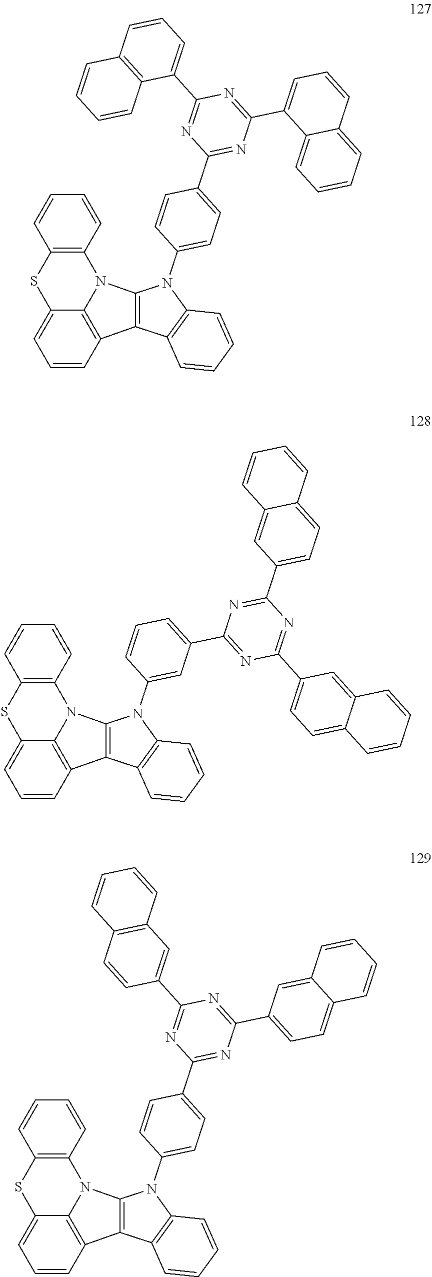 [SMILES: c1ccc2c(c1)Sc1cccc3c4c5ccccc5n(-c5ccc(-c6nc(-c7cccc8ccccc78)nc(-c7cccc8ccccc78)n6)cc5)c4n-2c13 ; c1cc(-c2nc(-c3ccc4ccccc4c3)nc(-c3ccc4ccccc4c3)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc8ccccc8c7)nc(-c7ccc8ccccc8c7)n6)cc5)c4n-2c13]

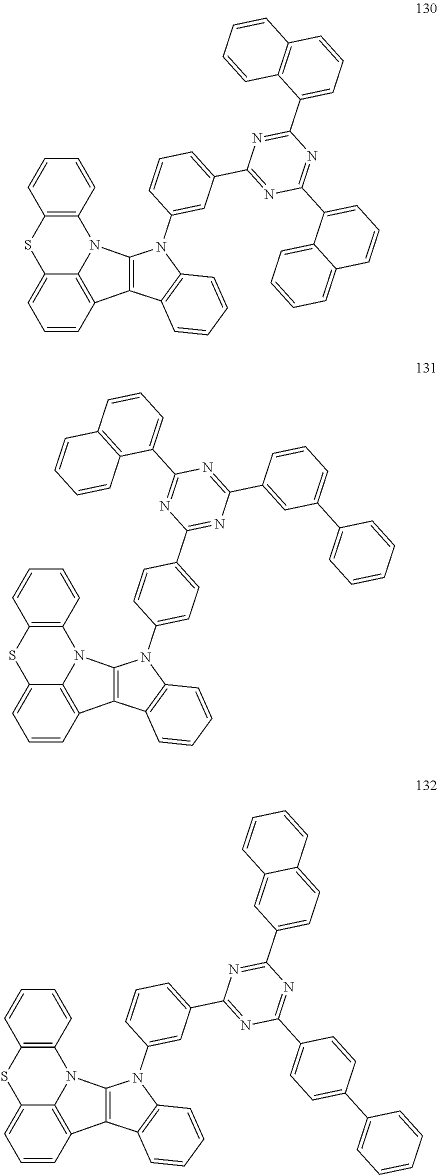 [SMILES: c1cc(-c2nc(-c3cccc4ccccc34)nc(-c3cccc4ccccc34)n2)cc(-n2c3ccccc3c3c4cccc5c4n(c32)-c2ccccc2S5)c1 ; c1ccc(-c2cccc(-c3nc(-c4ccc(-n5c6ccccc6c6c7cccc8c7n(c65)-c5ccccc5S8)cc4)nc(-c4cccc5ccccc45)n3)c2)cc1 ; c1ccc(-c2ccc(-c3nc(-c4cccc(-n5c6ccccc6c6c7cccc8c7n(c65)-c5ccccc5S8)c4)nc(-c4ccc5ccccc5c4)n3)cc2)cc1]

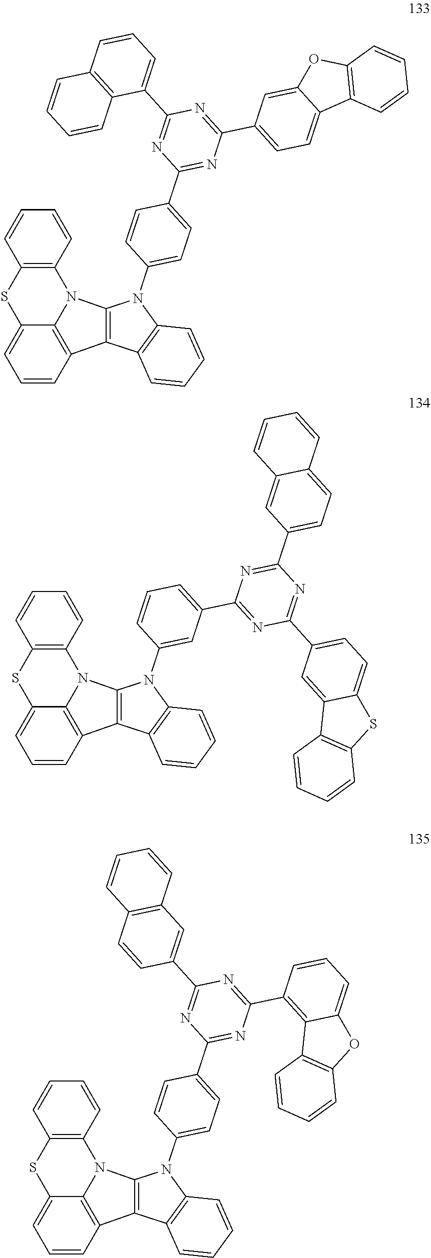 [SMILES: c1ccc2c(c1)Sc1cccc3c4c5ccccc5n(-c5ccc(-c6nc(-c7ccc8c(c7)oc7ccccc78)nc(-c7cccc8ccccc78)n6)cc5)c4n-2c13 ; c1cc(-c2nc(-c3ccc4ccccc4c3)nc(-c3ccc4sc5ccccc5c4c3)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc8ccccc8c7)nc(-c7cccc8oc9ccccc9c78)n6)cc5)c4n-2c13]

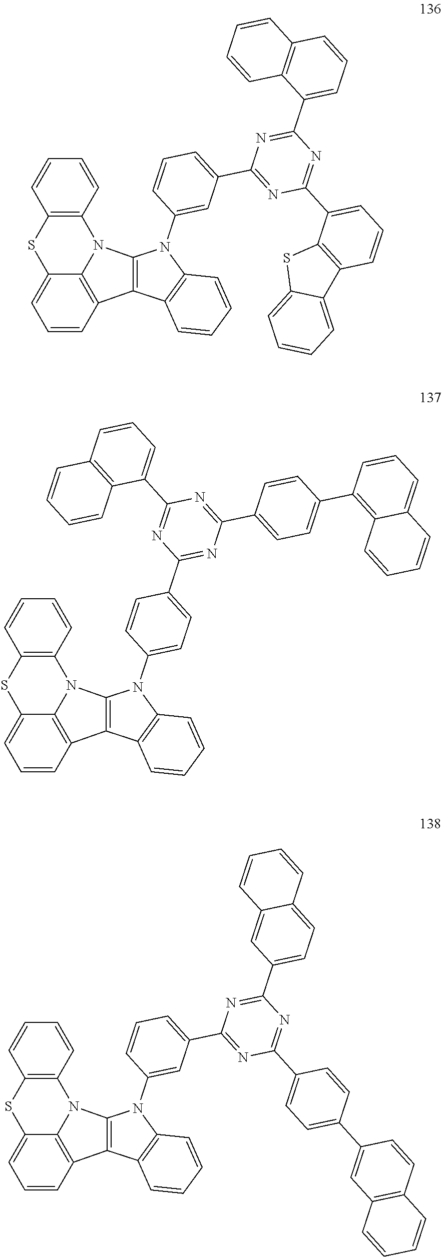 [SMILES: c1cc(-c2nc(-c3cccc4ccccc34)nc(-c3cccc4c3sc3ccccc34)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc(-c8cccc9ccccc89)cc7)nc(-c7cccc8ccccc78)n6)cc5)c4n-2c13 ; c1cc(-c2nc(-c3ccc(-c4ccc5ccccc5c4)cc3)nc(-c3ccc4ccccc4c3)n2)cc(-n2c3ccccc3c3c4cccc5c4n(c32)-c2ccccc2S5)c1]

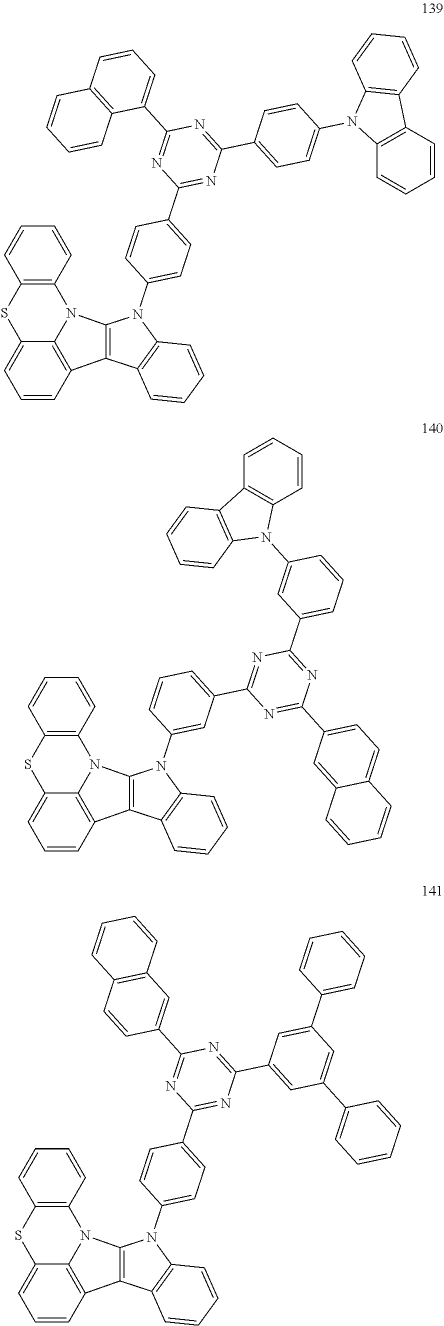 [SMILES: c1ccc2c(c1)Sc1cccc3c4c5ccccc5n(-c5ccc(-c6nc(-c7ccc(-n8c9ccccc9c9ccccc98)cc7)nc(-c7cccc8ccccc78)n6)cc5)c4n-2c13 ; c1cc(-c2nc(-c3cccc(-n4c5ccccc5c5c6cccc7c6n(c54)-c4ccccc4S7)c3)nc(-c3ccc4ccccc4c3)n2)cc(-n2c3ccccc3c3ccccc32)c1 ; c1ccc(-c2cc(-c3ccccc3)cc(-c3nc(-c4ccc(-n5c6ccccc6c6c7cccc8c7n(c65)-c5ccccc5S8)cc4)nc(-c4ccc5ccccc5c4)n3)c2)cc1]

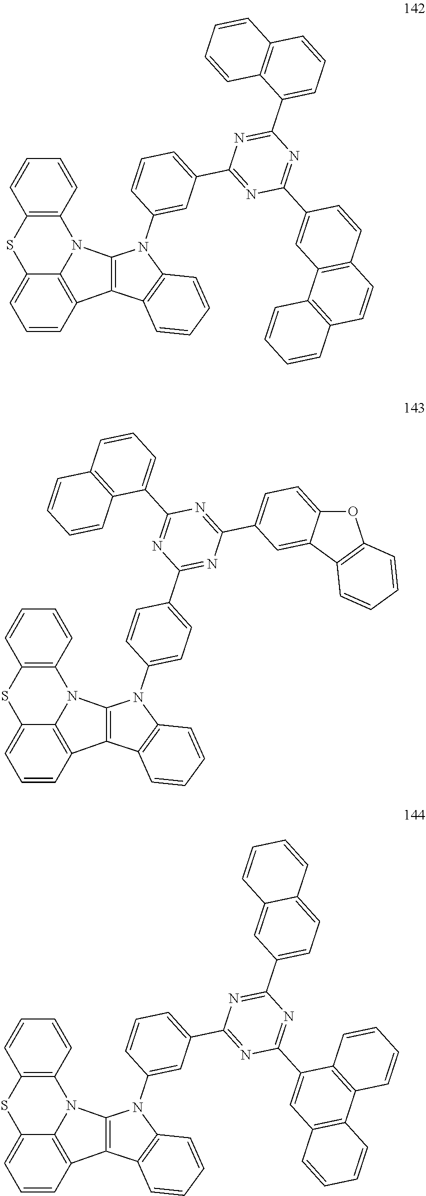 [SMILES: c1cc(-c2nc(-c3ccc4ccc5ccccc5c4c3)nc(-c3cccc4ccccc34)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc8oc9ccccc9c8c7)nc(-c7cccc8ccccc78)n6)cc5)c4n-2c13 ; c1cc(-c2nc(-c3ccc4ccccc4c3)nc(-c3cc4ccccc4c4ccccc34)n2)cc(-n2c3ccccc3c3c4cccc5c4n(c32)-c2ccccc2S5)c1]

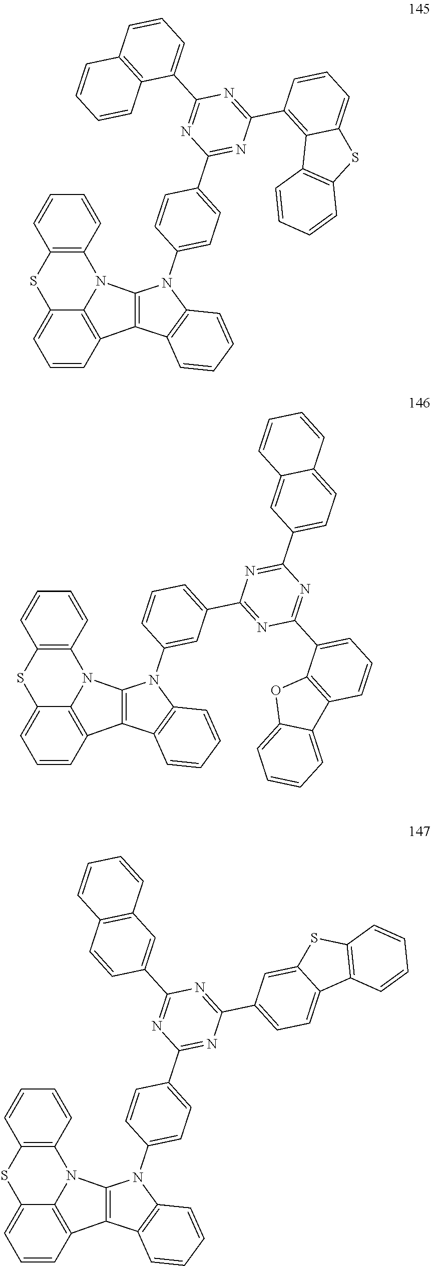 [SMILES: c1ccc2c(c1)Sc1cccc3c4c5ccccc5n(-c5ccc(-c6nc(-c7cccc8ccccc78)nc(-c7cccc8sc9ccccc9c78)n6)cc5)c4n-2c13 ; c1cc(-c2nc(-c3ccc4ccccc4c3)nc(-c3cccc4c3oc3ccccc34)n2)cc(-n2c3ccccc3c3c4cccc5c4n(c32)-c2ccccc2S5)c1 ; c1ccc2c(c1)Sc1cccc3c4c5ccccc5n(-c5ccc(-c6nc(-c7ccc8ccccc8c7)nc(-c7ccc8c(c7)sc7ccccc78)n6)cc5)c4n-2c13]

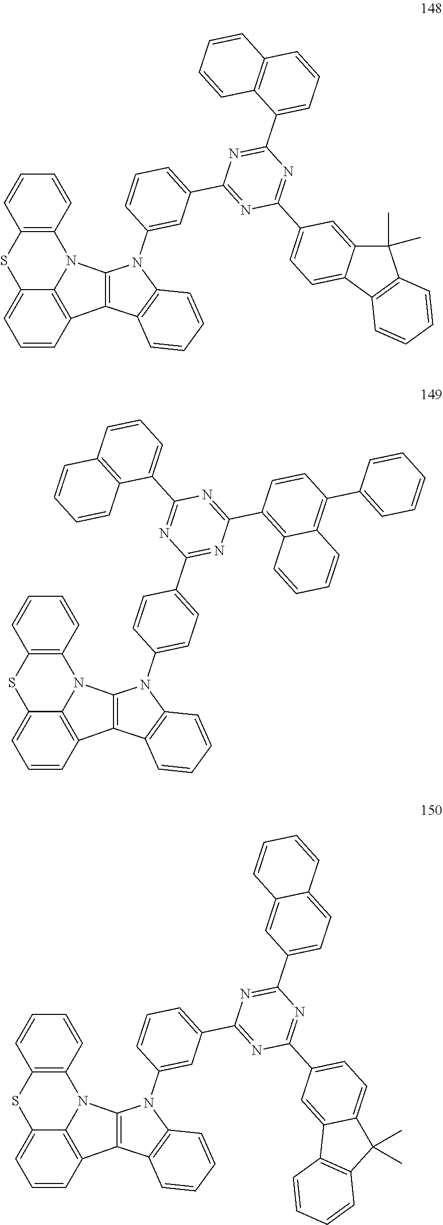 [SMILES: CC1(C)c2ccccc2-c2ccc(-c3nc(-c4cccc(-n5c6ccccc6c6c7cccc8c7n(c65)-c5ccccc5S8)c4)nc(-c4cccc5ccccc45)n3)cc21 ; c1ccc(-c2ccc(-c3nc(-c4ccc(-n5c6ccccc6c6c7cccc8c7n(c65)-c5ccccc5S8)cc4)nc(-c4cccc5ccccc45)n3)c3ccccc23)cc1 ; CC1(C)c2ccccc2-c2cc(-c3nc(-c4cccc(-n5c6ccccc6c6c7cccc8c7n(c65)-c5ccccc5S8)c4)nc(-c4ccc5ccccc5c4)n3)ccc21]

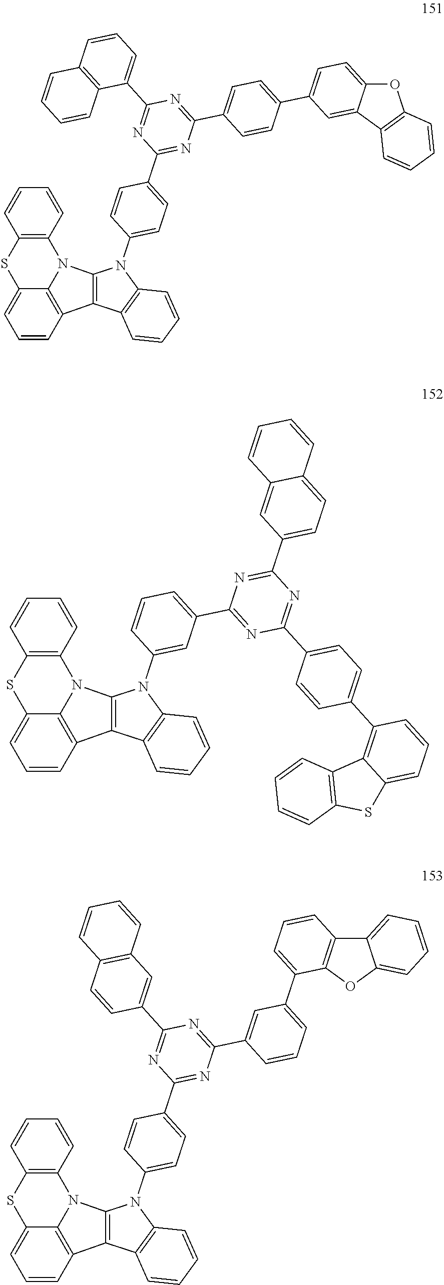 [SMILES: c1ccc2c(c1)Sc1cccc3c4c5ccccc5n(-c5ccc(-c6nc(-c7ccc(-c8ccc9oc%10ccccc%10c9c8)cc7)nc(-c7cccc8ccccc78)n6)cc5)c4n-2c13 ; c1cc(-c2nc(-c3ccc(-c4cccc5sc6ccccc6c45)cc3)nc(-c3ccc4ccccc4c3)n2)cc(-n2c3ccccc3c3c4cccc5c4n(c32)-c2ccccc2S5)c1 ; c1cc(-c2nc(-c3ccc(-n4c5ccccc5c5c6cccc7c6n(c54)-c4ccccc4S7)cc3)nc(-c3ccc4ccccc4c3)n2)cc(-c2cccc3c2oc2ccccc23)c1]

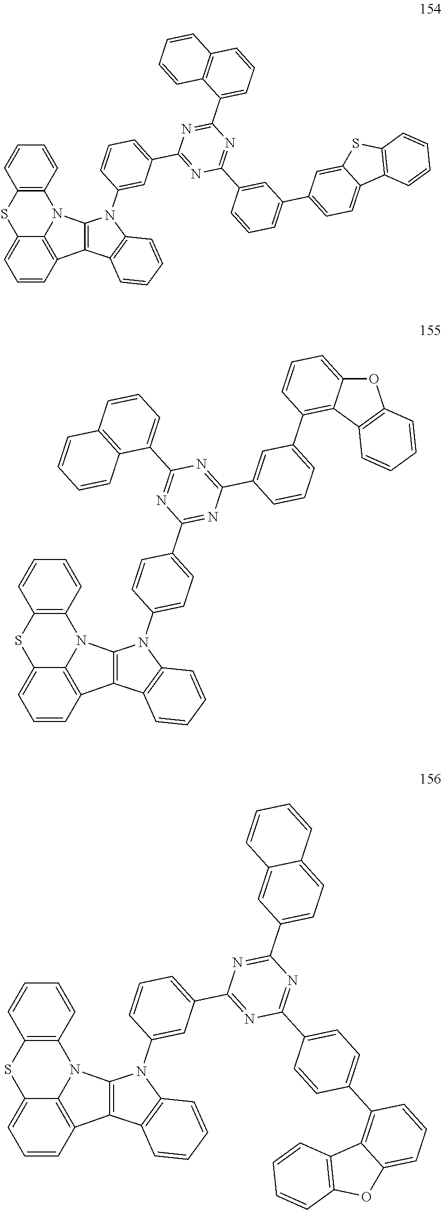 [SMILES: c1cc(-c2ccc3c(c2)sc2ccccc23)cc(-c2nc(-c3cccc(-n4c5ccccc5c5c6cccc7c6n(c54)-c4ccccc4S7)c3)nc(-c3cccc4ccccc34)n2)c1 ; c1cc(-c2nc(-c3ccc(-n4c5ccccc5c5c6cccc7c6n(c54)-c4ccccc4S7)cc3)nc(-c3cccc4ccccc34)n2)cc(-c2cccc3oc4ccccc4c23)c1 ; c1cc(-c2nc(-c3ccc(-c4cccc5oc6ccccc6c45)cc3)nc(-c3ccc4ccccc4c3)n2)cc(-n2c3ccccc3c3c4cccc5c4n(c32)-c2ccccc2S5)c1]

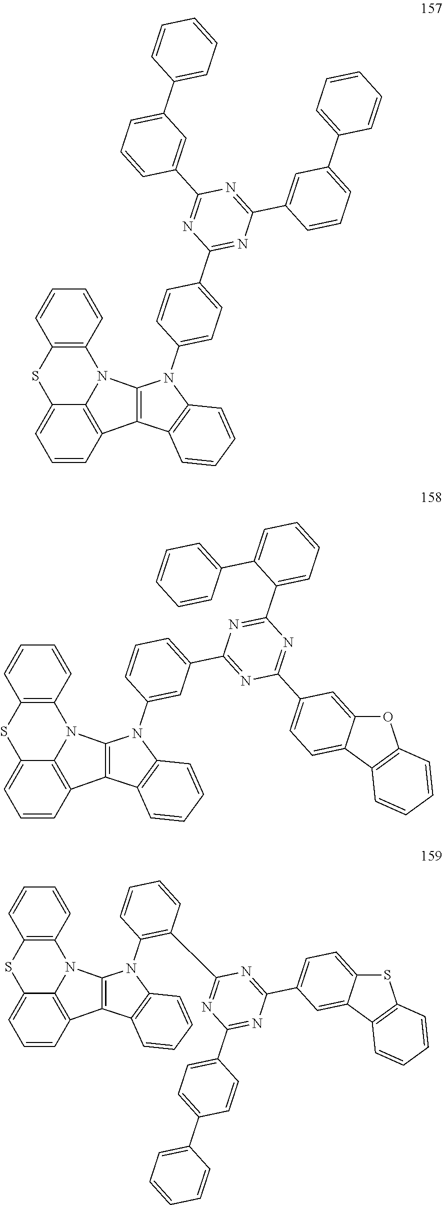 [SMILES: c1ccc(-c2cccc(-c3nc(-c4ccc(-n5c6ccccc6c6c7cccc8c7n(c65)-c5ccccc5S8)cc4)nc(-c4cccc(-c5ccccc5)c4)n3)c2)cc1 ; c1ccc(-c2ccccc2-c2nc(-c3cccc(-n4c5ccccc5c5c6cccc7c6n(c54)-c4ccccc4S7)c3)nc(-c3ccc4c(c3)oc3ccccc34)n2)cc1 ; c1ccc(-c2ccc(-c3nc(-c4ccc5sc6ccccc6c5c4)nc(-c4ccccc4-n4c5ccccc5c5c6cccc7c6n(c54)-c4ccccc4S7)n3)cc2)cc1]

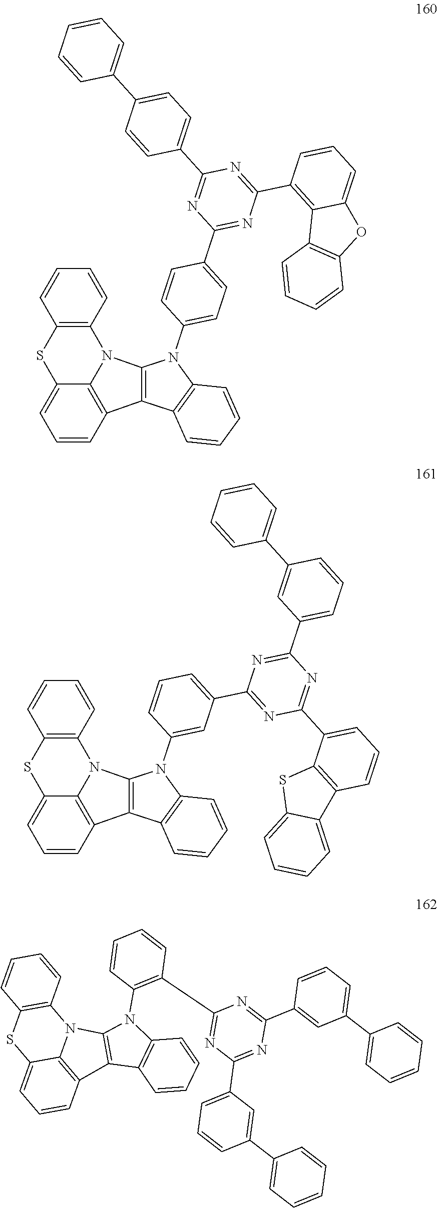

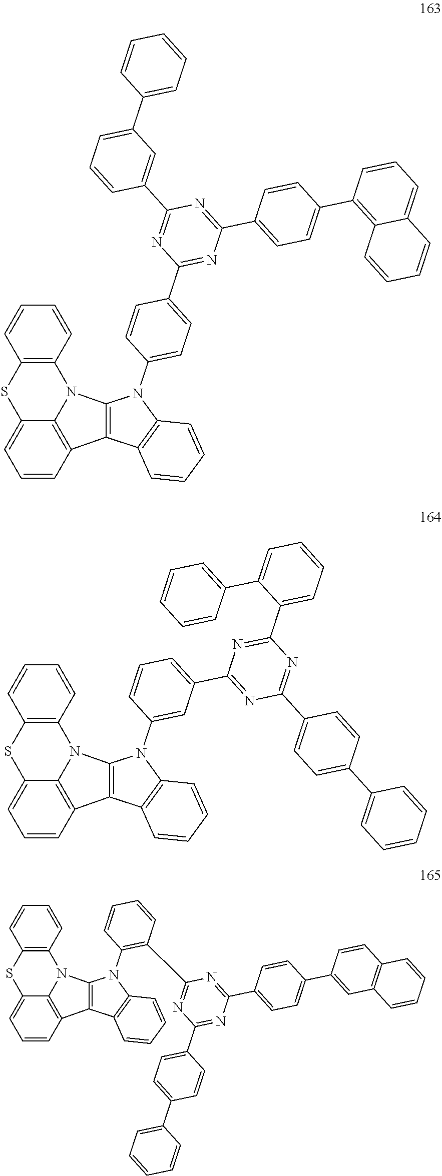 [SMILES: c1ccc(-c2cccc(-c3nc(-c4ccc(-c5cccc6ccccc56)cc4)nc(-c4ccc(-n5c6ccccc6c6c7cccc8c7n(c65)-c5ccccc5S8)cc4)n3)c2)cc1 ; c1ccc(-c2ccc(-c3nc(-c4cccc(-n5c6ccccc6c6c7cccc8c7n(c65)-c5ccccc5S8)c4)nc(-c4ccccc4-c4ccccc4)n3)cc2)cc1 ; c1ccc(-c2ccc(-c3nc(-c4ccc(-c5ccc6ccccc6c5)cc4)nc(-c4ccccc4-n4c5ccccc5c5c6cccc7c6n(c54)-c4ccccc4S7)n3)cc2)cc1]

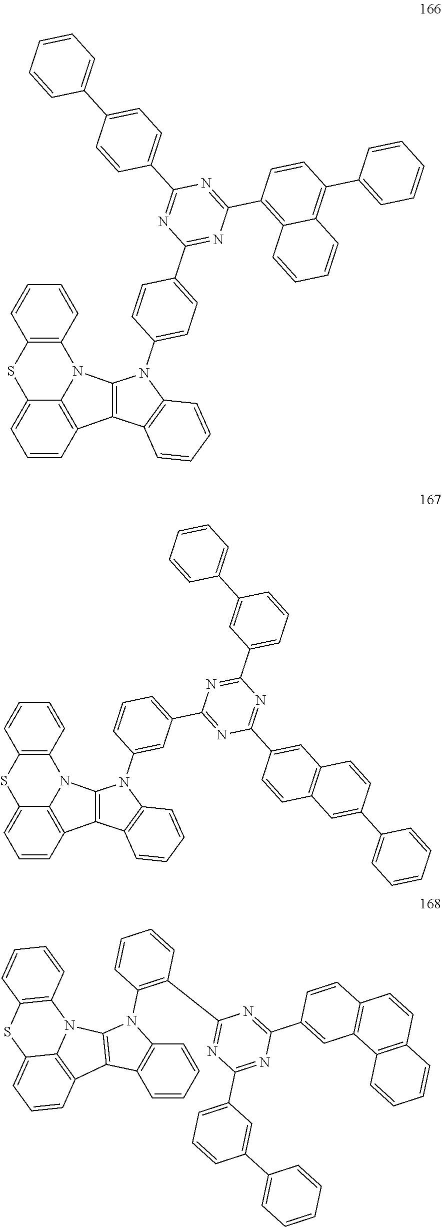 [SMILES: c1ccc(-c2ccc(-c3nc(-c4ccc(-n5c6ccccc6c6c7cccc8c7n(c65)-c5ccccc5S8)cc4)nc(-c4ccc(-c5ccccc5)c5ccccc45)n3)cc2)cc1 ; c1ccc(-c2cccc(-c3nc(-c4cccc(-n5c6ccccc6c6c7cccc8c7n(c65)-c5ccccc5S8)c4)nc(-c4ccc5cc(-c6ccccc6)ccc5c4)n3)c2)cc1 ; c1ccc(-c2cccc(-c3nc(-c4ccc5ccc6ccccc6c5c4)nc(-c4ccccc4-n4c5ccccc5c5c6cccc7c6n(c54)-c4ccccc4S7)n3)c2)cc1]

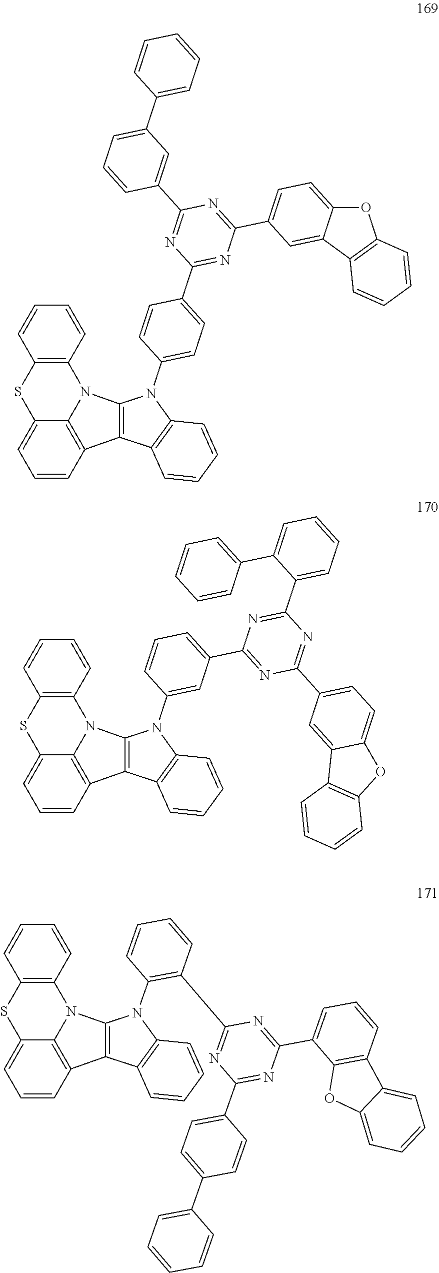 [SMILES: c1ccc(-c2cccc(-c3nc(-c4ccc(-n5c6ccccc6c6c7cccc8c7n(c65)-c5ccccc5S8)cc4)nc(-c4ccc5oc6ccccc6c5c4)n3)c2)cc1 ; c1ccc(-c2ccccc2-c2nc(-c3cccc(-n4c5ccccc5c5c6cccc7c6n(c54)-c4ccccc4S7)c3)nc(-c3ccc4oc5ccccc5c4c3)n2)cc1 ; c1ccc(-c2ccc(-c3nc(-c4ccccc4-n4c5ccccc5c5c6cccc7c6n(c54)-c4ccccc4S7)nc(-c4cccc5c4oc4ccccc45)n3)cc2)cc1]

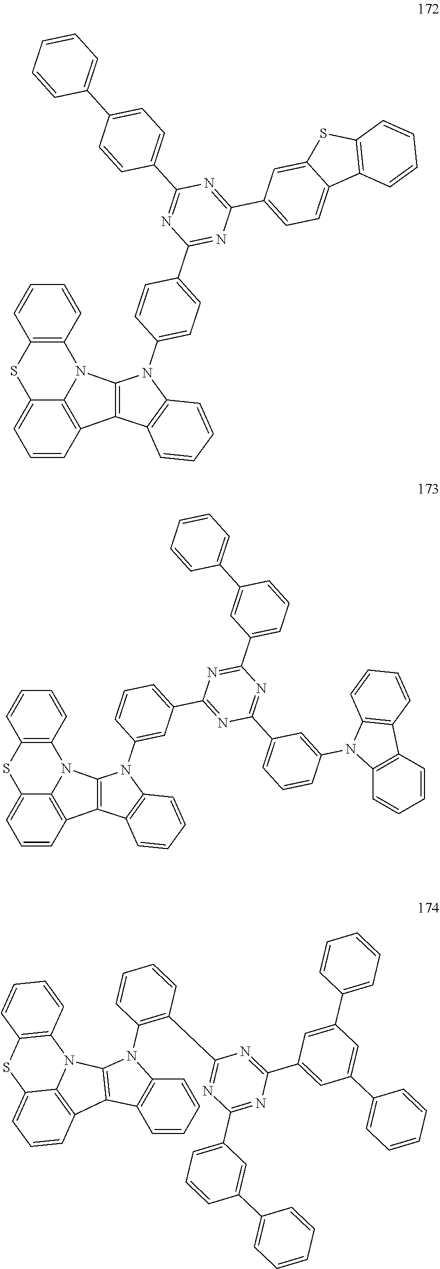 [SMILES: c1ccc(-c2ccc(-c3nc(-c4ccc(-n5c6ccccc6c6c7cccc8c7n(c65)-c5ccccc5S8)cc4)nc(-c4ccc5c(c4)sc4ccccc45)n3)cc2)cc1 ; c1ccc(-c2cccc(-c3nc(-c4cccc(-n5c6ccccc6c6ccccc65)c4)nc(-c4cccc(-n5c6ccccc6c6c7cccc8c7n(c65)-c5ccccc5S8)c4)n3)c2)cc1 ; c1ccc(-c2cccc(-c3nc(-c4cc(-c5ccccc5)cc(-c5ccccc5)c4)nc(-c4ccccc4-n4c5ccccc5c5c6cccc7c6n(c54)-c4ccccc4S7)n3)c2)cc1]

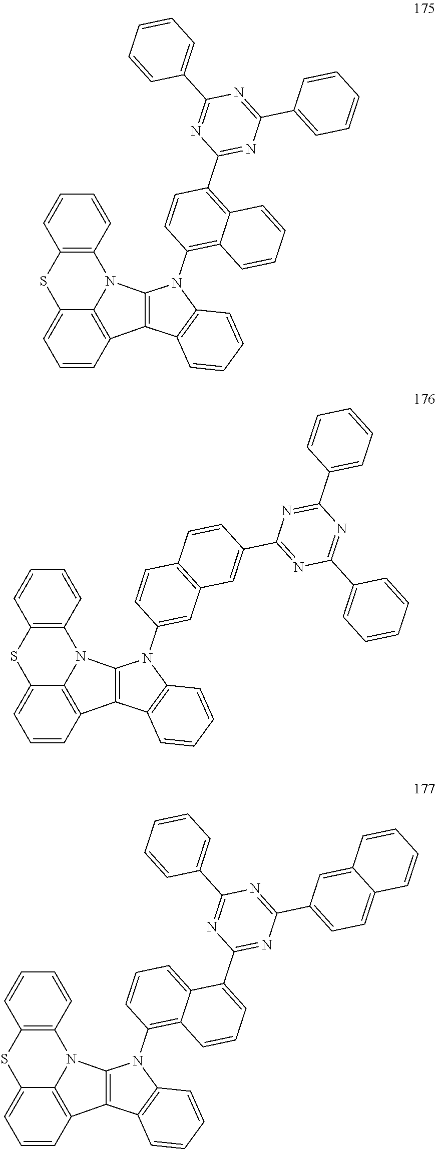 [SMILES: c1ccc(-c2nc(-c3ccccc3)nc(-c3ccc(-n4c5ccccc5c5c6cccc7c6n(c54)-c4ccccc4S7)c4ccccc34)n2)cc1 ; c1ccc(-c2nc(-c3ccccc3)nc(-c3ccc4ccc(-n5c6ccccc6c6c7cccc8c7n(c65)-c5ccccc5S8)cc4c3)n2)cc1 ; c1ccc(-c2nc(-c3ccc4ccccc4c3)nc(-c3cccc4c(-n5c6ccccc6c6c7cccc8c7n(c65)-c5ccccc5S8)cccc34)n2)cc1]

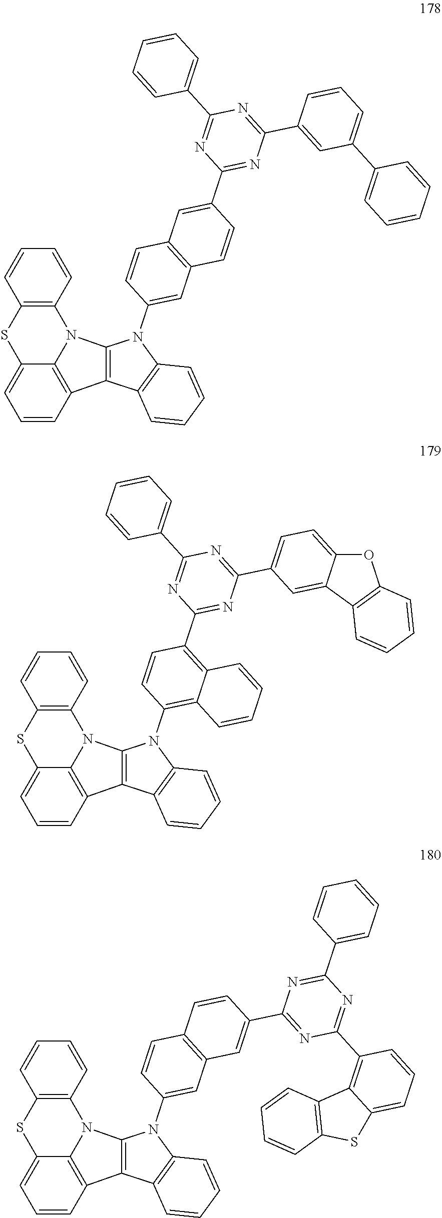 [SMILES: c1ccc(-c2cccc(-c3nc(-c4ccccc4)nc(-c4ccc5cc(-n6c7ccccc7c7c8cccc9c8n(c76)-c6ccccc6S9)ccc5c4)n3)c2)cc1 ; c1ccc(-c2nc(-c3ccc4oc5ccccc5c4c3)nc(-c3ccc(-n4c5ccccc5c5c6cccc7c6n(c54)-c4ccccc4S7)c4ccccc34)n2)cc1 ; c1ccc(-c2nc(-c3ccc4ccc(-n5c6ccccc6c6c7cccc8c7n(c65)-c5ccccc5S8)cc4c3)nc(-c3cccc4sc5ccccc5c34)n2)cc1]

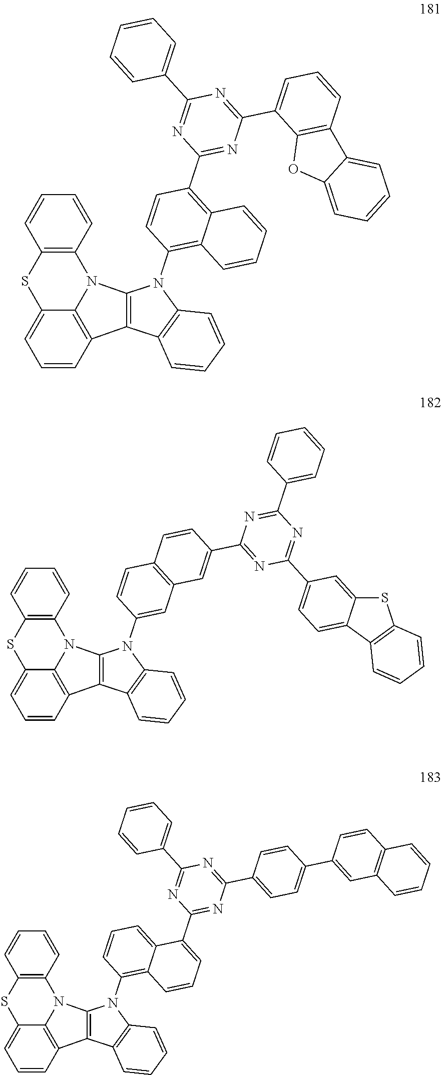 [SMILES: c1ccc(-c2nc(-c3ccc(-n4c5ccccc5c5c6cccc7c6n(c54)-c4ccccc4S7)c4ccccc34)nc(-c3cccc4c3oc3ccccc34)n2)cc1 ; c1ccc(-c2nc(-c3ccc4ccc(-n5c6ccccc6c6c7cccc8c7n(c65)-c5ccccc5S8)cc4c3)nc(-c3ccc4c(c3)sc3ccccc34)n2)cc1 ; c1ccc(-c2nc(-c3ccc(-c4ccc5ccccc5c4)cc3)nc(-c3cccc4c(-n5c6ccccc6c6c7cccc8c7n(c65)-c5ccccc5S8)cccc34)n2)cc1]

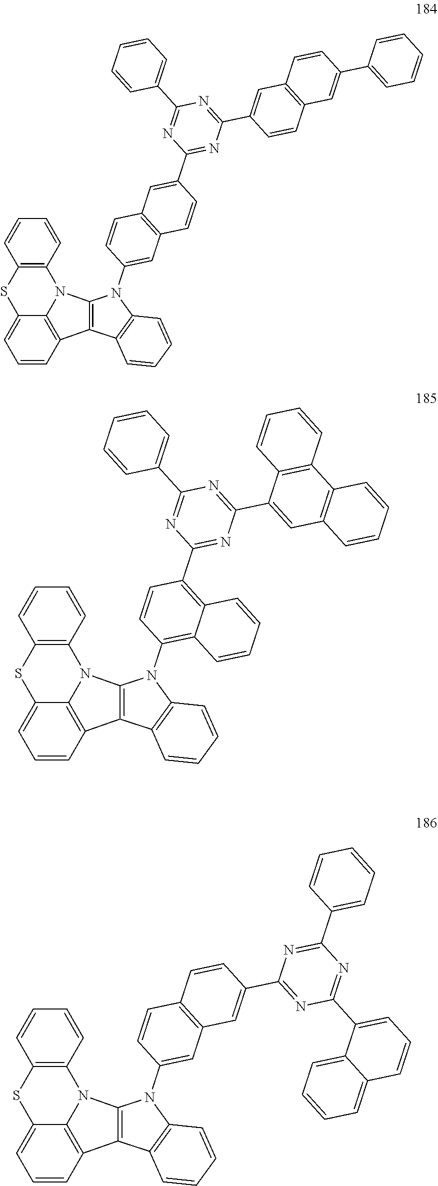

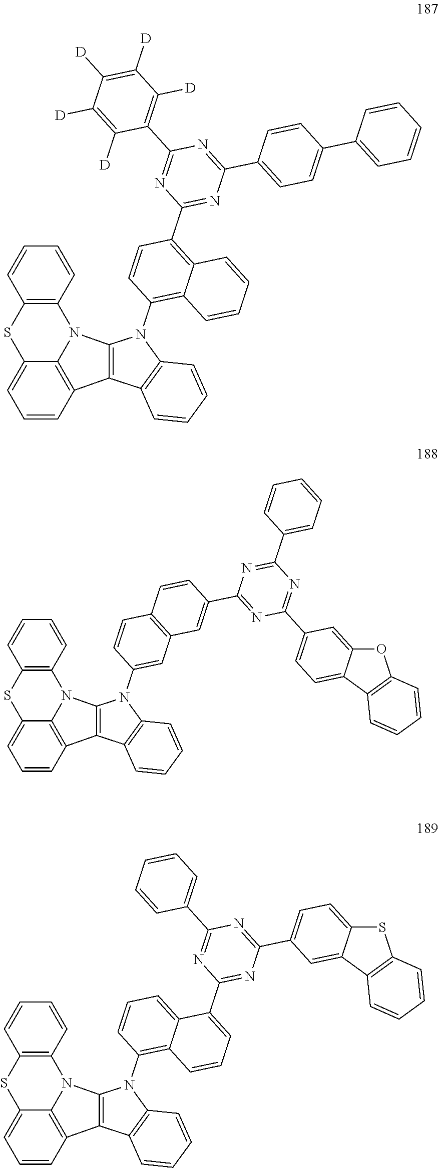 [SMILES: [2H]c1c([2H])c([2H])c(-c2nc(-c3ccc(-c4ccccc4)cc3)nc(-c3ccc(-n4c5ccccc5c5c6cccc7c6n(c54)-c4ccccc4S7)c4ccccc34)n2)c([2H])c1[2H] ; c1ccc(-c2nc(-c3ccc4ccc(-n5c6ccccc6c6c7cccc8c7n(c65)-c5ccccc5S8)cc4c3)nc(-c3ccc4c(c3)oc3ccccc34)n2)cc1 ; c1ccc(-c2nc(-c3ccc4sc5ccccc5c4c3)nc(-c3cccc4c(-n5c6ccccc6c6c7cccc8c7n(c65)-c5ccccc5S8)cccc34)n2)cc1]

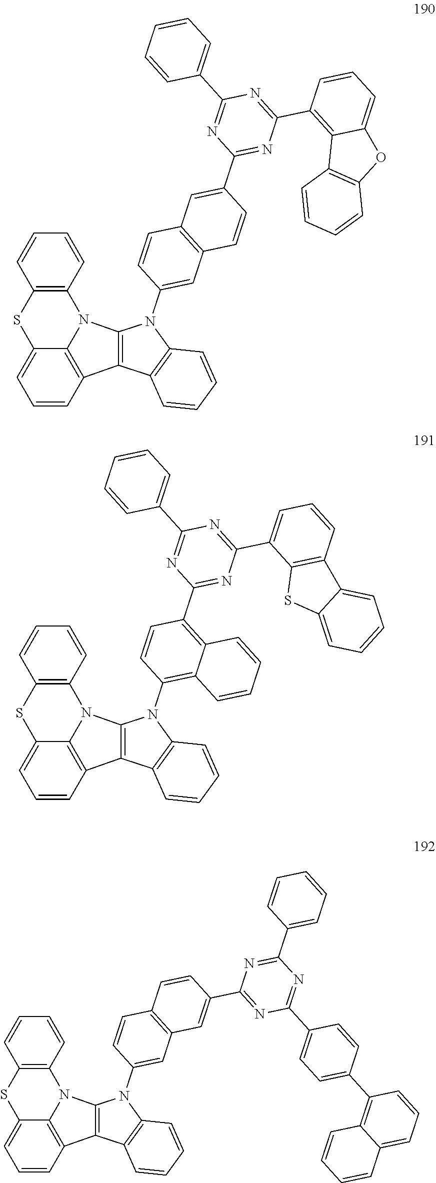 [SMILES: c1ccc(-c2nc(-c3ccc4cc(-n5c6ccccc6c6c7cccc8c7n(c65)-c5ccccc5S8)ccc4c3)nc(-c3cccc4oc5ccccc5c34)n2)cc1 ; c1ccc(-c2nc(-c3ccc(-n4c5ccccc5c5c6cccc7c6n(c54)-c4ccccc4S7)c4ccccc34)nc(-c3cccc4c3sc3ccccc34)n2)cc1 ; c1ccc(-c2nc(-c3ccc(-c4cccc5ccccc45)cc3)nc(-c3ccc4ccc(-n5c6ccccc6c6c7cccc8c7n(c65)-c5ccccc5S8)cc4c3)n2)cc1]

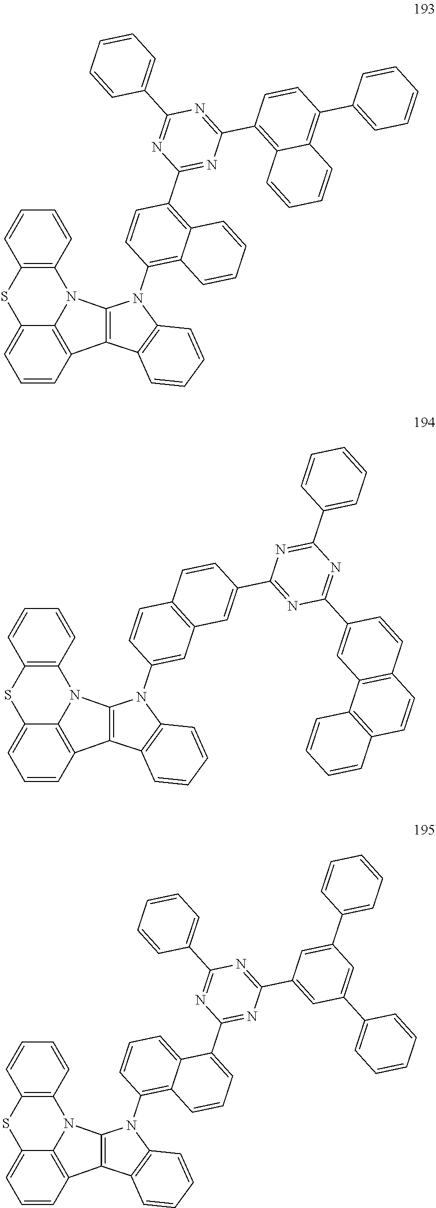 [SMILES: c1ccc(-c2nc(-c3ccc(-c4ccccc4)c4ccccc34)nc(-c3ccc(-n4c5ccccc5c5c6cccc7c6n(c54)-c4ccccc4S7)c4ccccc34)n2)cc1 ; c1ccc(-c2nc(-c3ccc4ccc(-n5c6ccccc6c6c7cccc8c7n(c65)-c5ccccc5S8)cc4c3)nc(-c3ccc4ccc5ccccc5c4c3)n2)cc1 ; c1ccc(-c2cc(-c3ccccc3)cc(-c3nc(-c4ccccc4)nc(-c4cccc5c(-n6c7ccccc7c7c8cccc9c8n(c76)-c6ccccc6S9)cccc45)n3)c2)cc1]

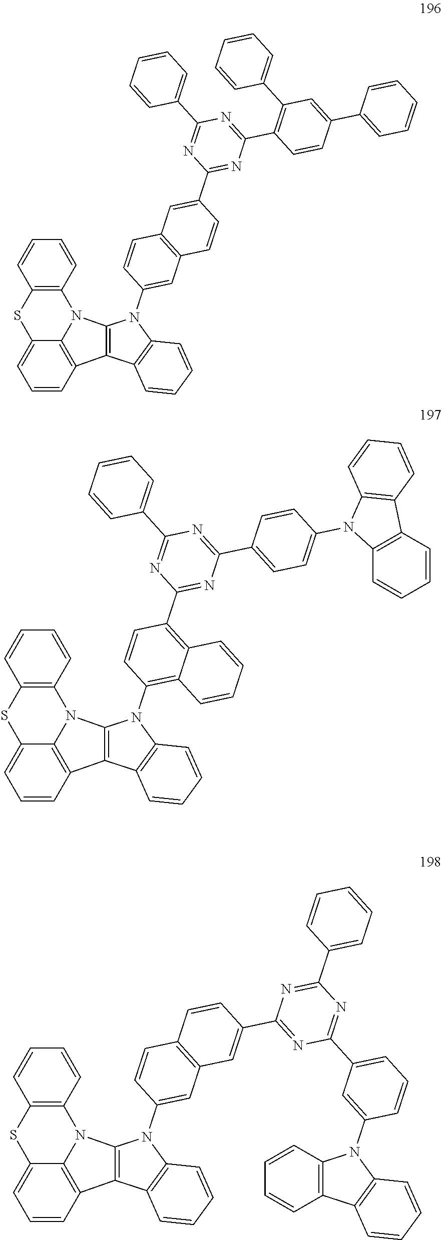 [SMILES: c1ccc(-c2ccc(-c3nc(-c4ccccc4)nc(-c4ccc5cc(-n6c7ccccc7c7c8cccc9c8n(c76)-c6ccccc6S9)ccc5c4)n3)c(-c3ccccc3)c2)cc1 ; c1ccc(-c2nc(-c3ccc(-n4c5ccccc5c5ccccc54)cc3)nc(-c3ccc(-n4c5ccccc5c5c6cccc7c6n(c54)-c4ccccc4S7)c4ccccc34)n2)cc1 ; c1ccc(-c2nc(-c3cccc(-n4c5ccccc5c5ccccc54)c3)nc(-c3ccc4ccc(-n5c6ccccc6c6c7cccc8c7n(c65)-c5ccccc5S8)cc4c3)n2)cc1]

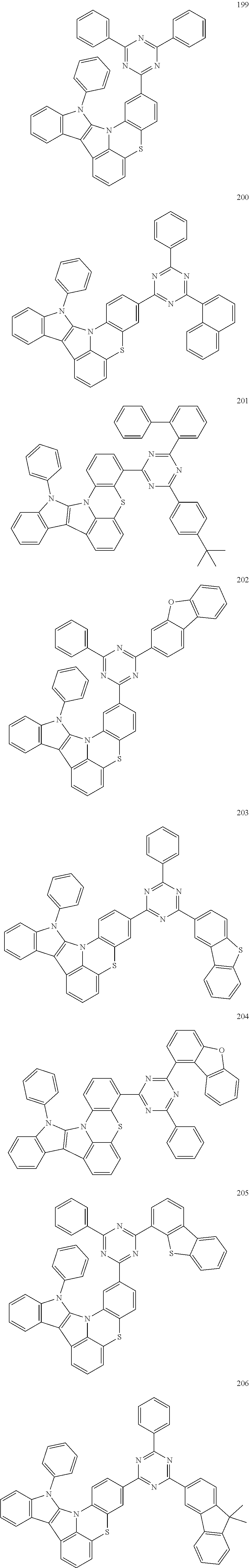

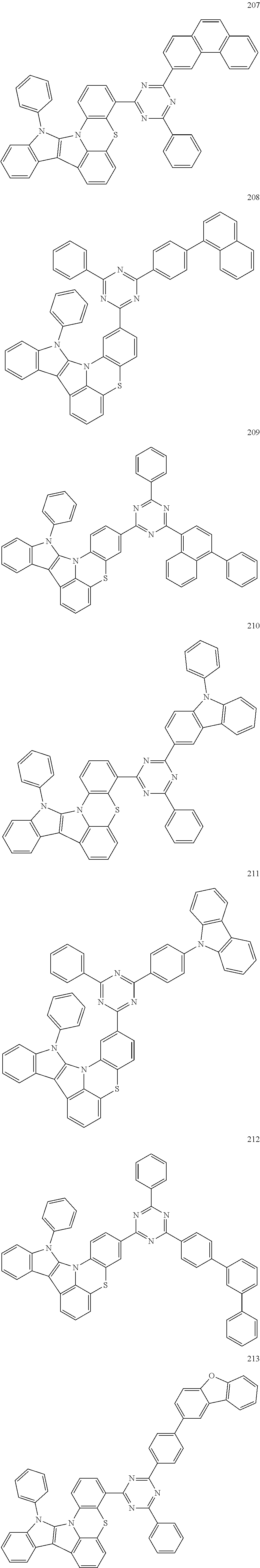

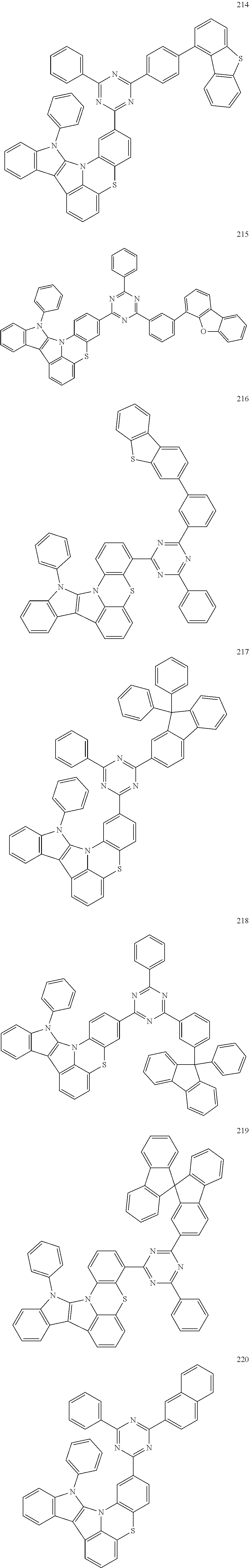

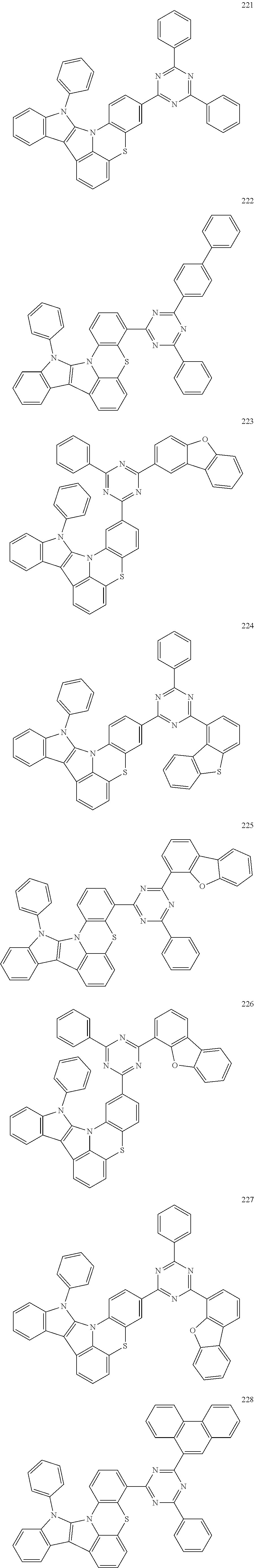

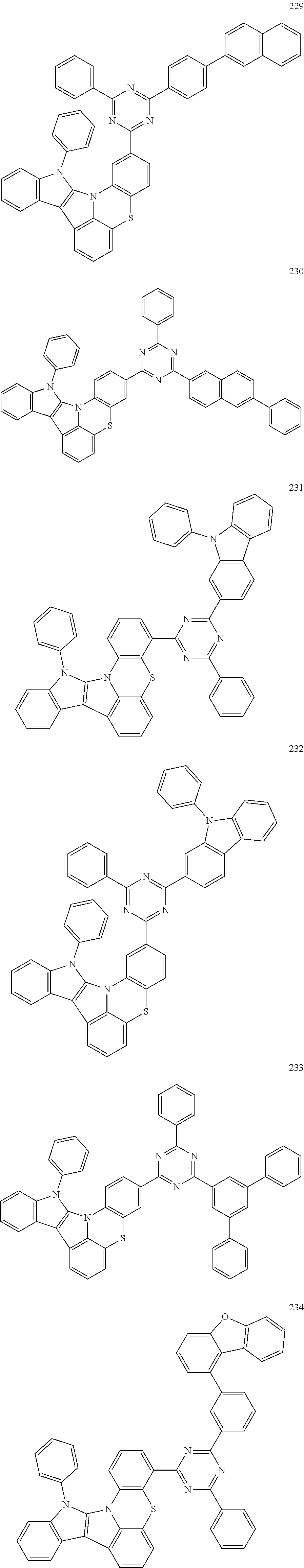

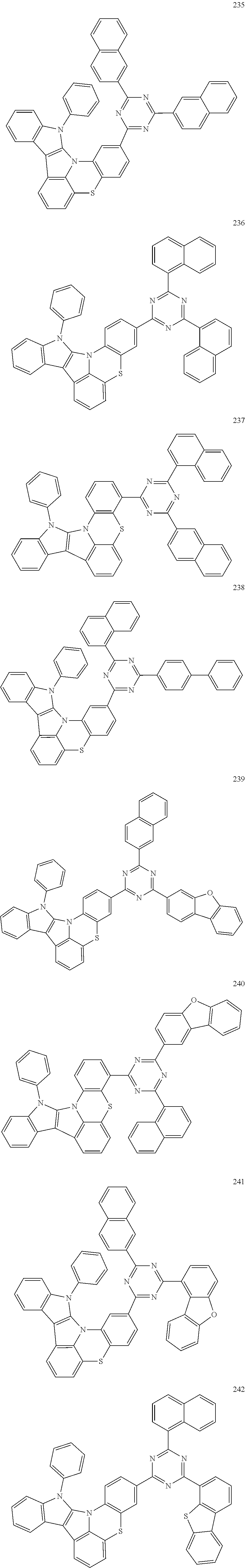

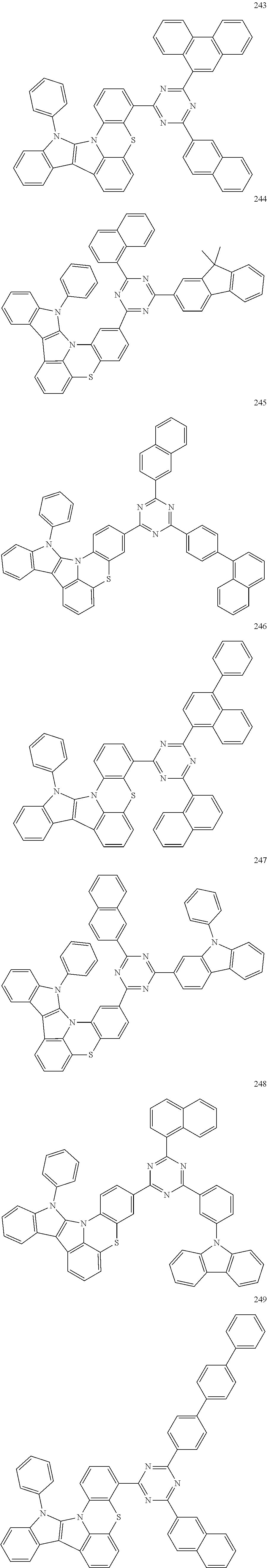

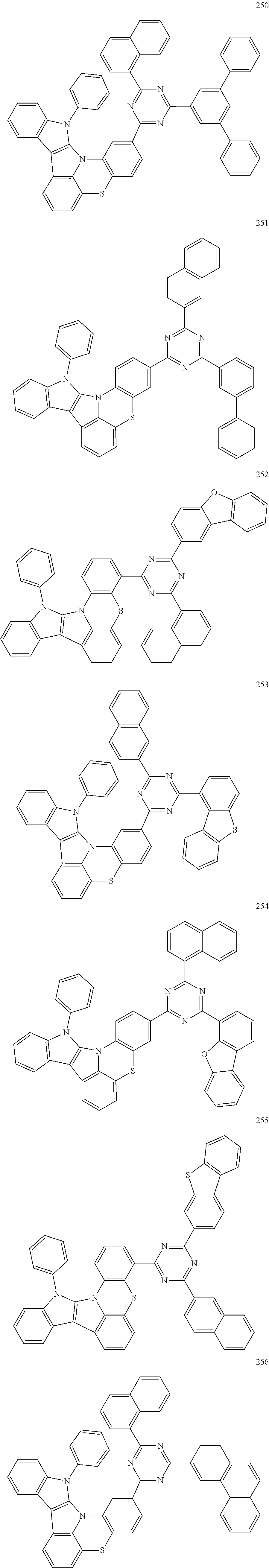

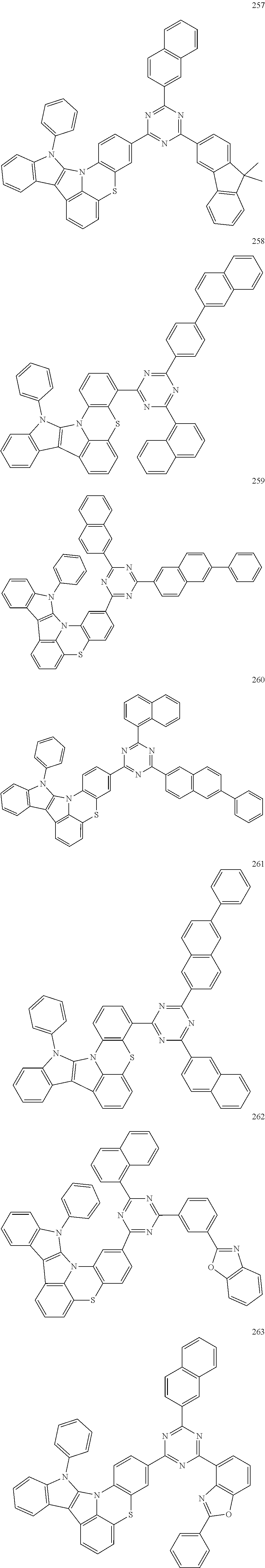

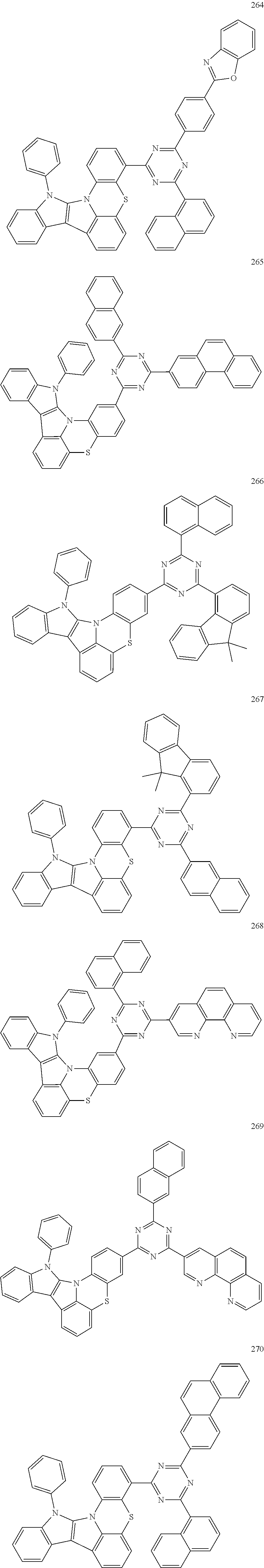

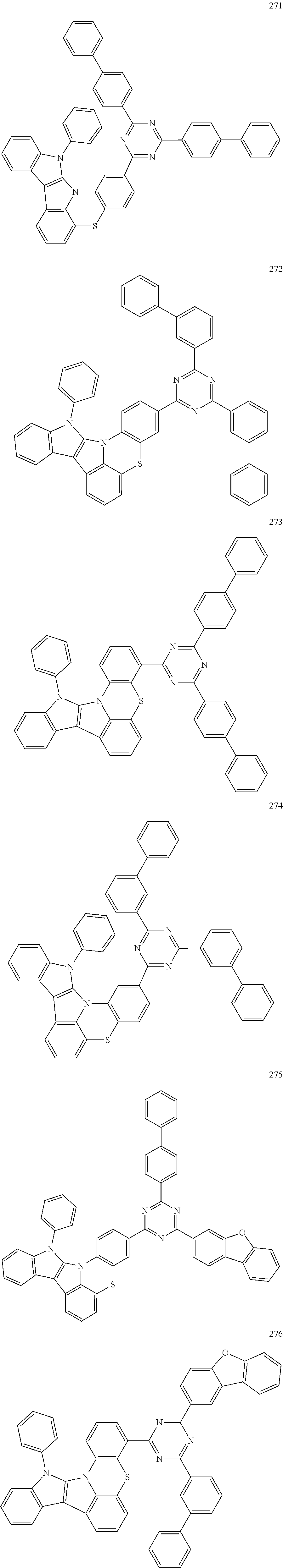

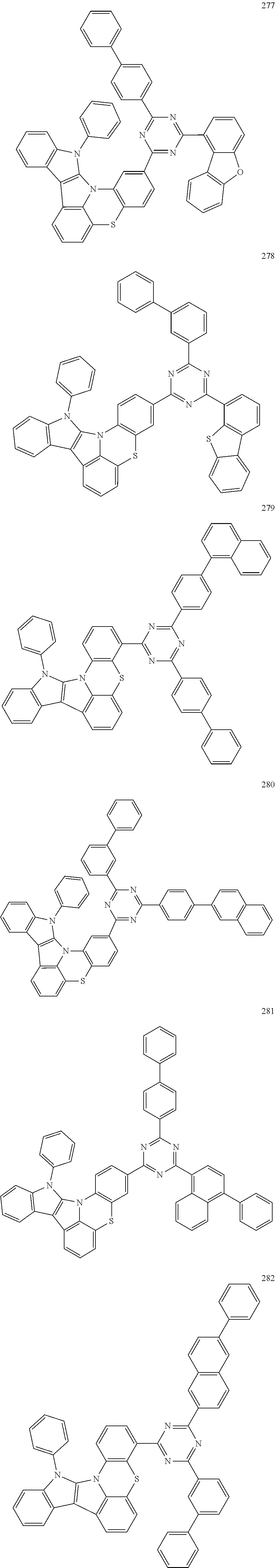

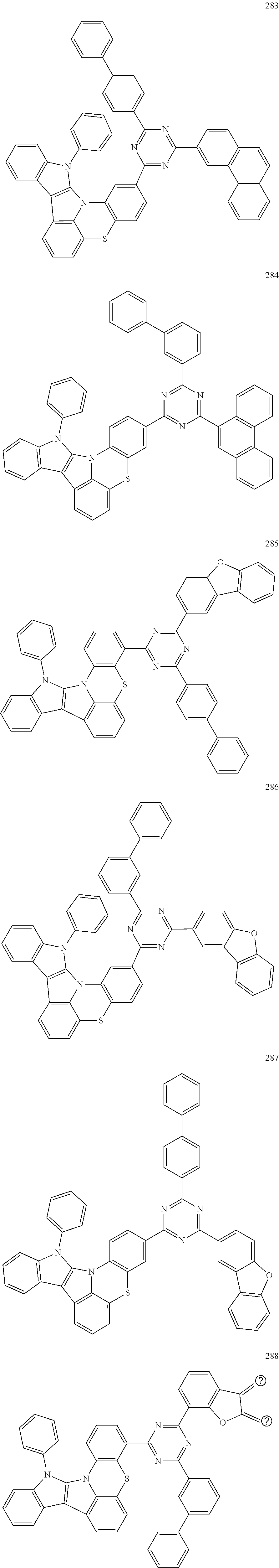

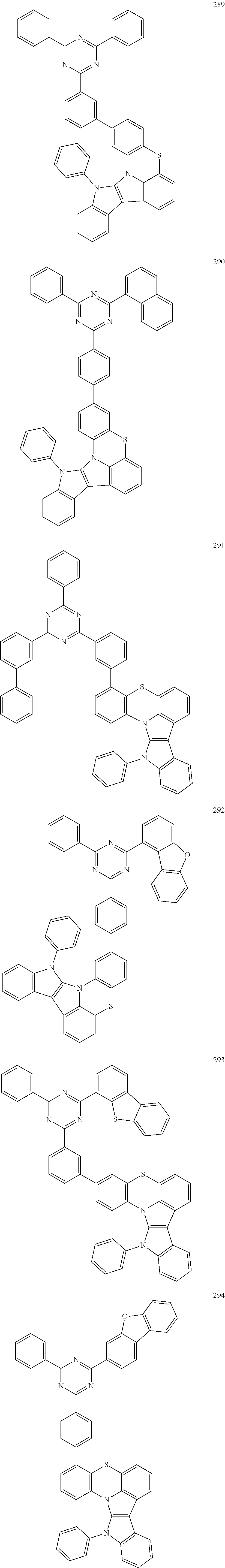

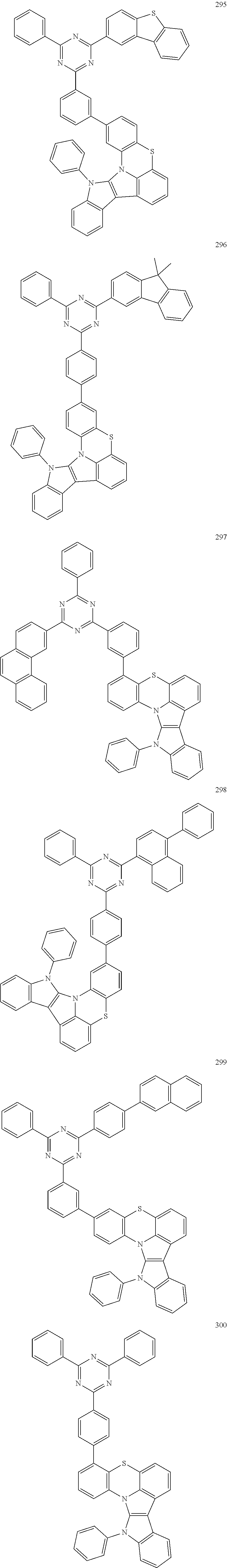

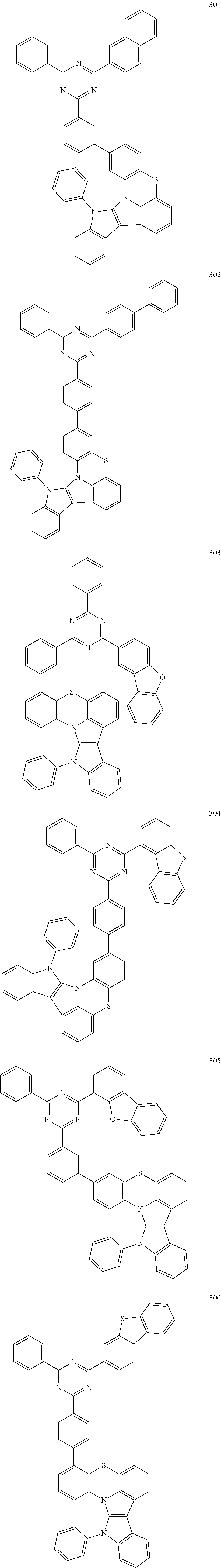

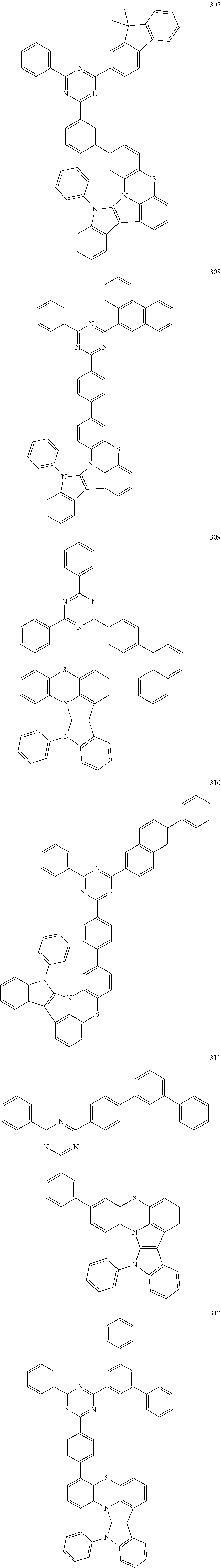

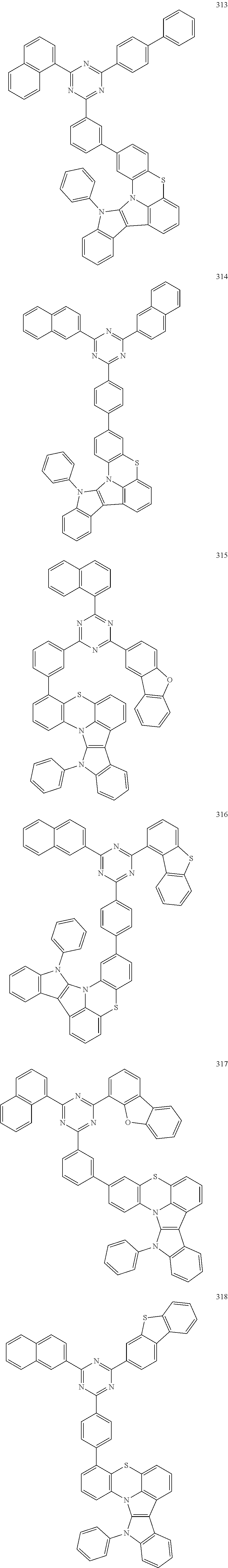

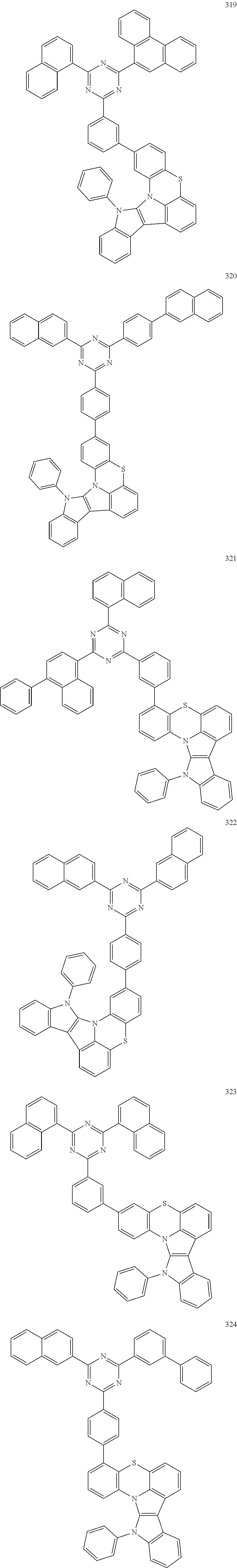

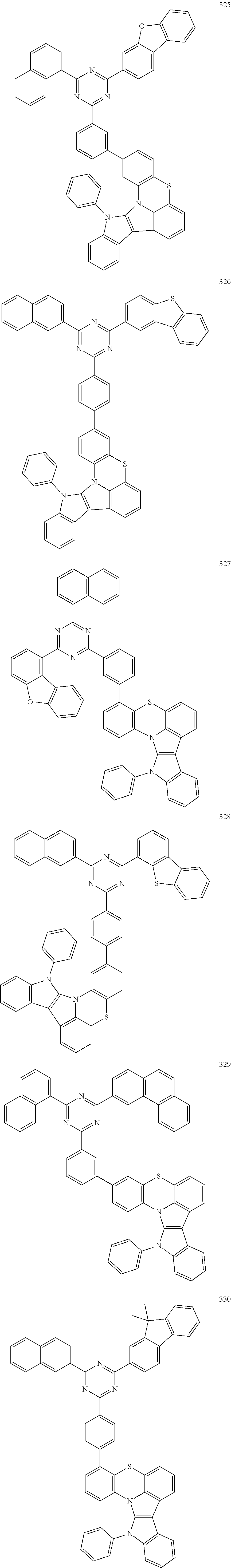

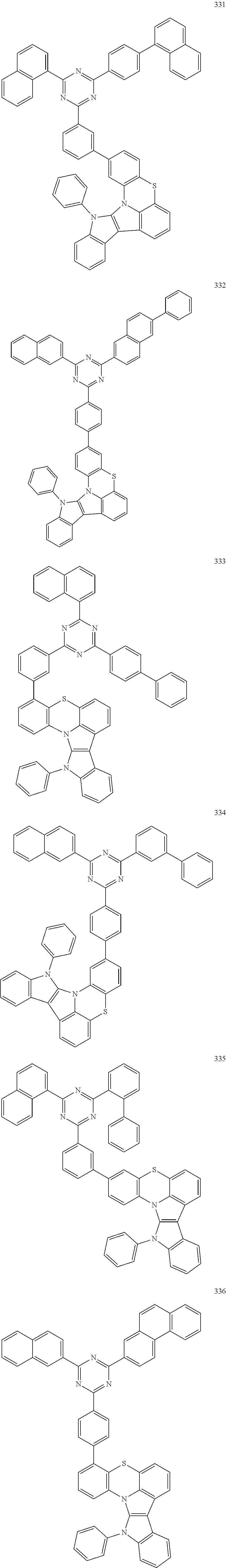

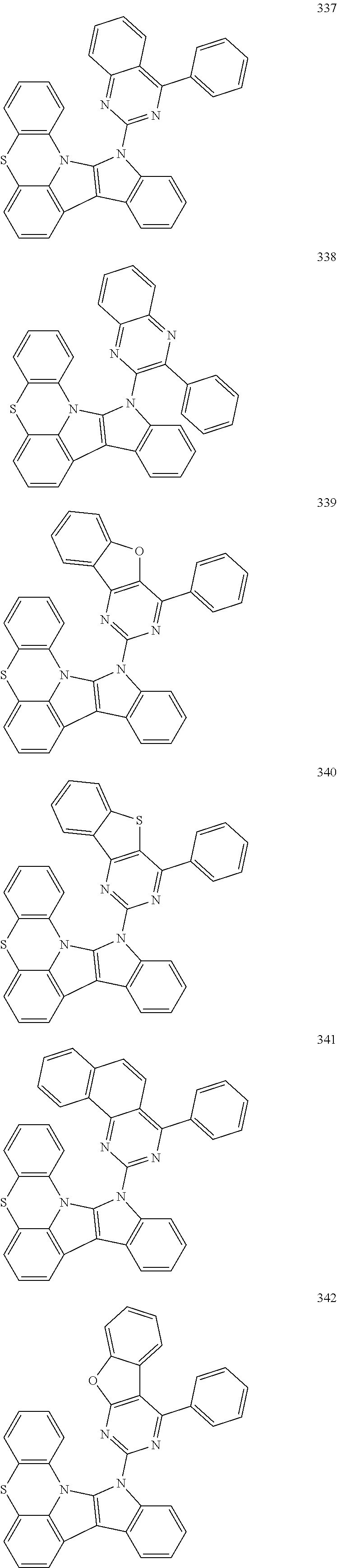

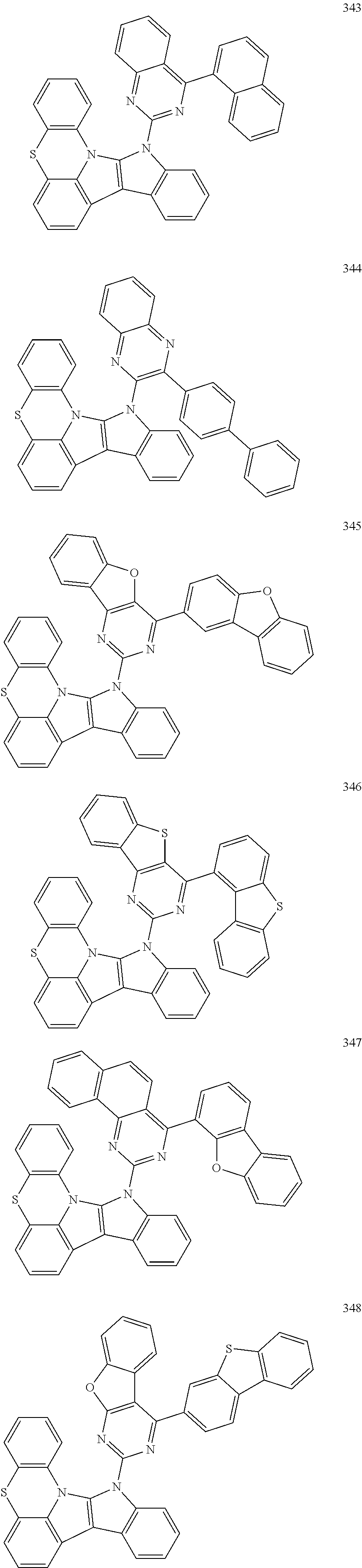

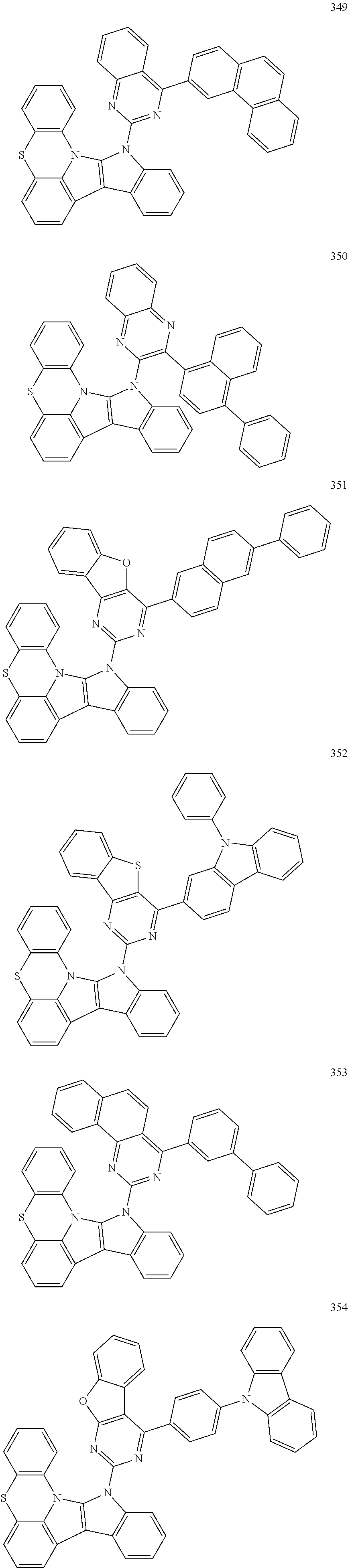

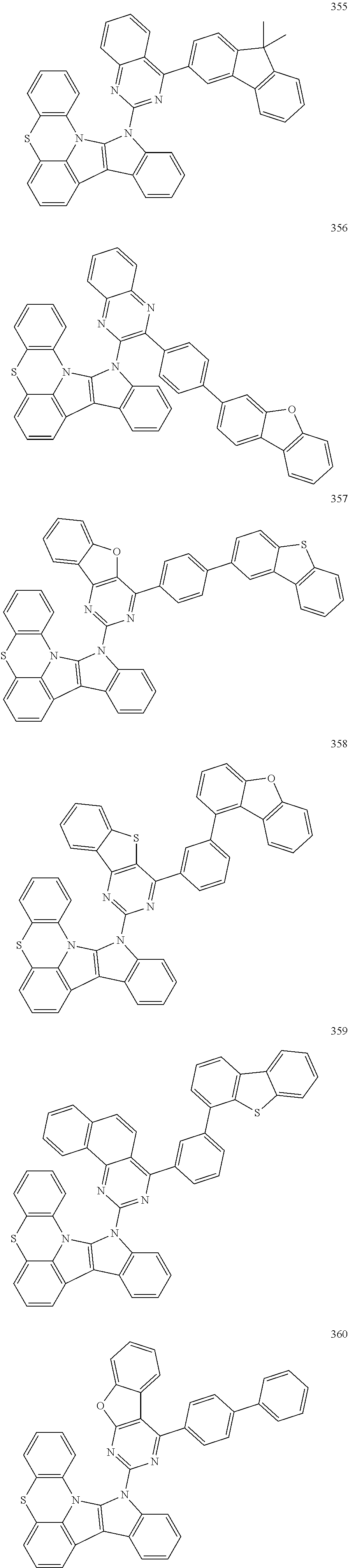

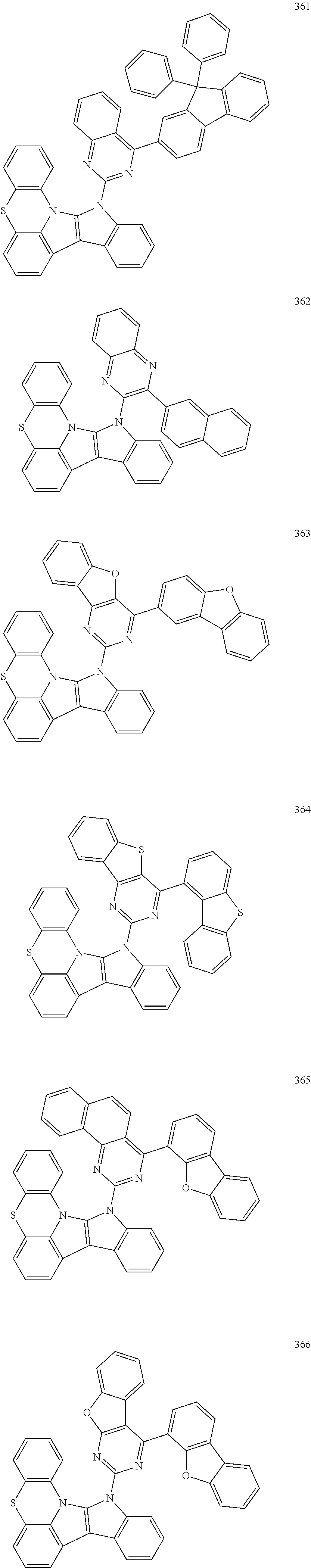

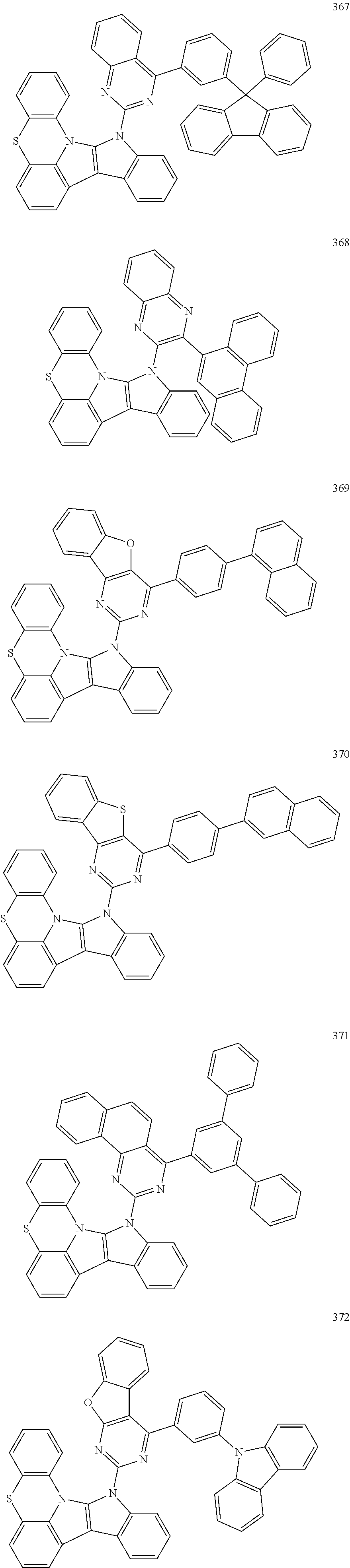

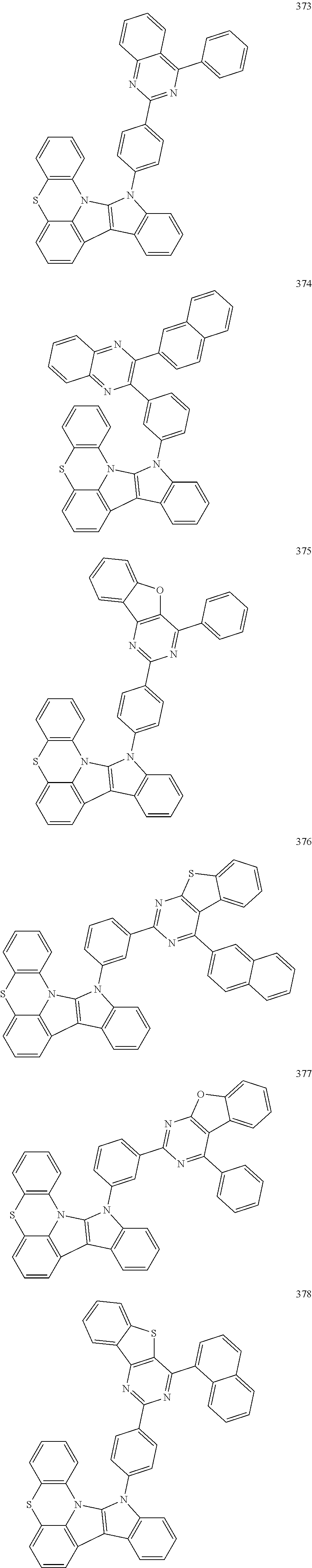

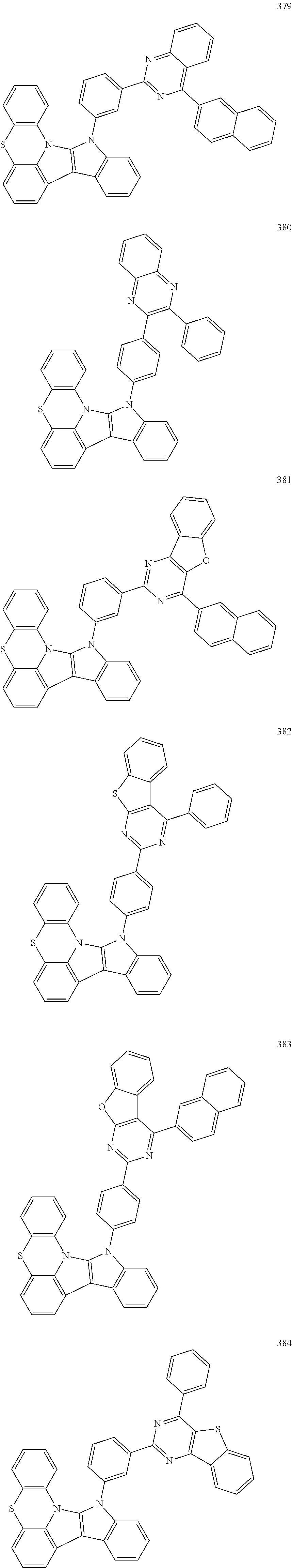

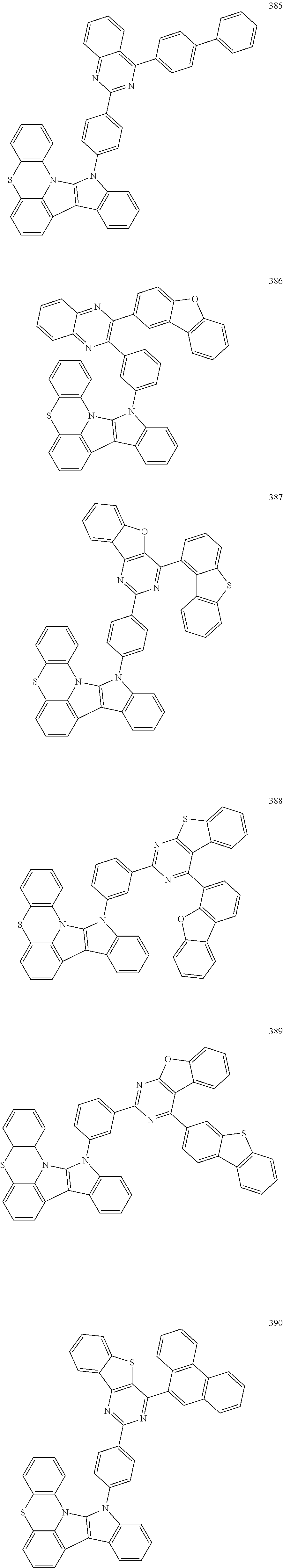

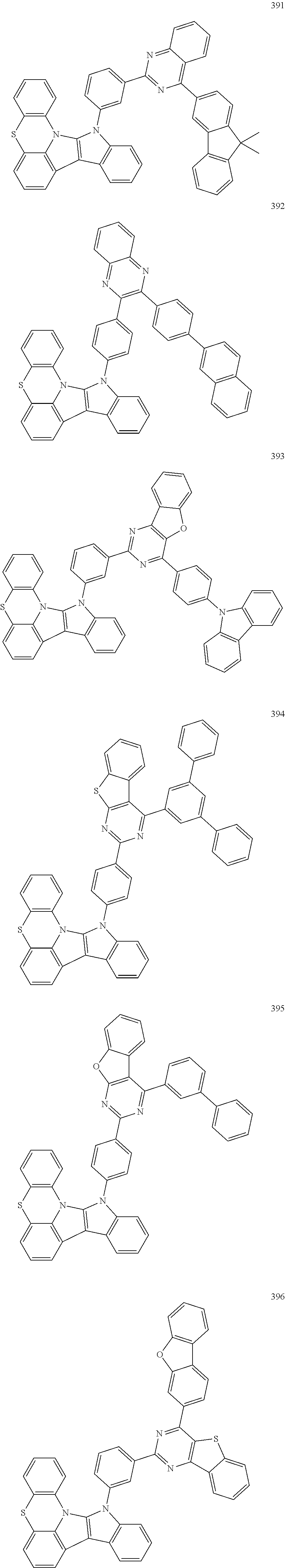

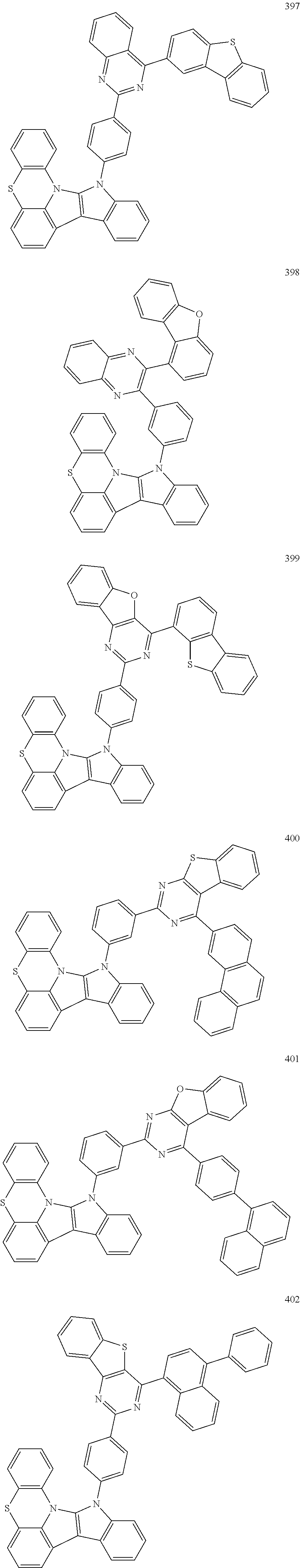

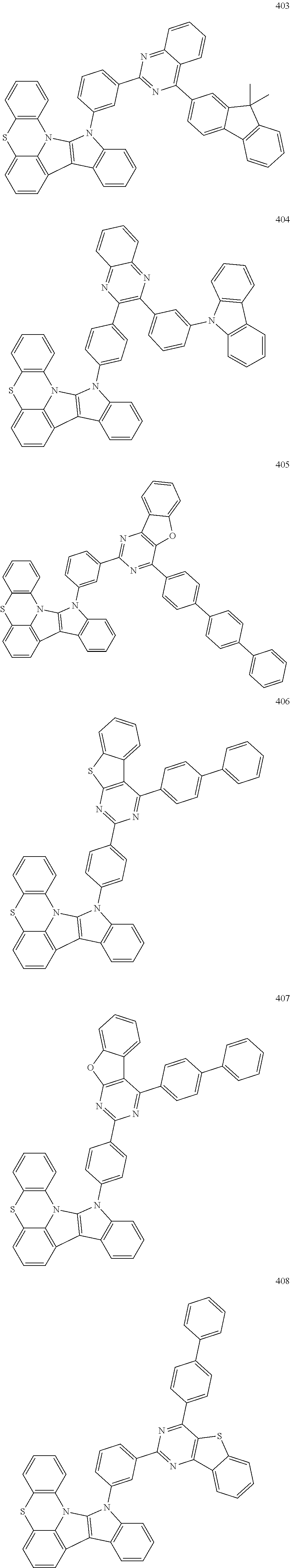

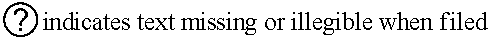

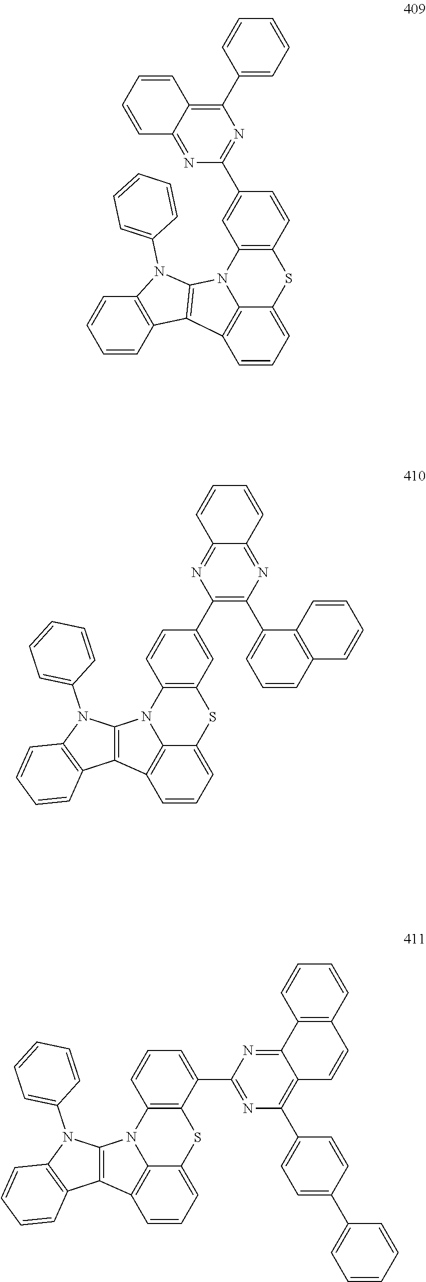 [SMILES: c1ccc(-c2nc(-c3ccc4c(c3)-n3c5c(cccc5c5c6ccccc6n(-c6ccccc6)c53)S4)nc3ccccc23)cc1 ; c1ccc(-n2c3ccccc3c3c4cccc5c4n(c32)-c2ccc(-c3nc4ccccc4nc3-c3cccc4ccccc34)cc2S5)cc1 ; c1ccc(-c2ccc(-c3nc(-c4cccc5c4Sc4cccc6c7c8ccccc8n(-c8ccccc8)c7n-5c46)nc4c3ccc3ccccc34)cc2)cc1]

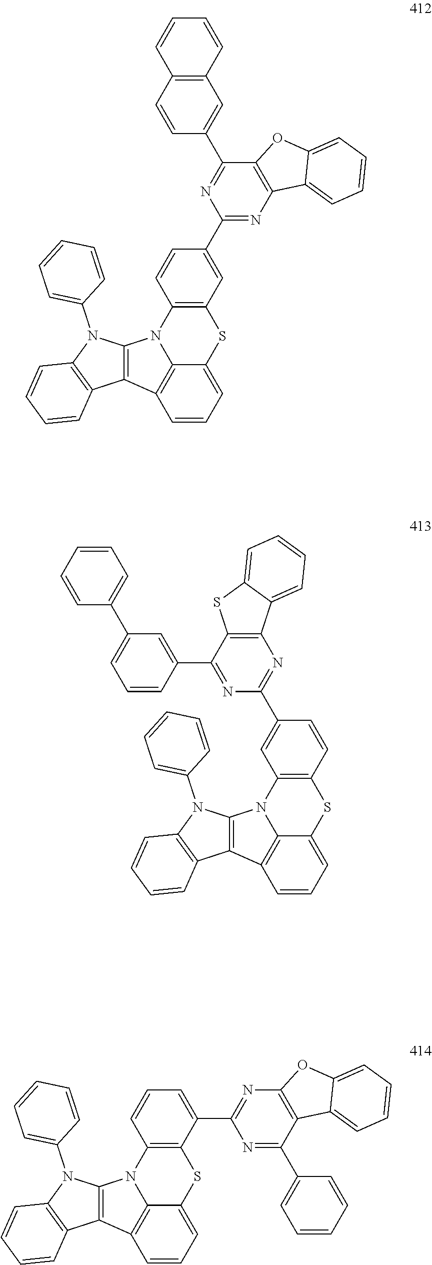 [SMILES: c1ccc(-n2c3ccccc3c3c4cccc5c4n(c32)-c2ccc(-c3nc(-c4ccc6ccccc6c4)c4oc6ccccc6c4n3)cc2S5)cc1 ; c1ccc(-c2cccc(-c3nc(-c4ccc5c(c4)-n4c6c(cccc6c6c7ccccc7n(-c7ccccc7)c64)S5)nc4c3sc3ccccc34)c2)cc1 ; c1ccc(-c2nc(-c3cccc4c3Sc3cccc5c6c7ccccc7n(-c7ccccc7)c6n-4c35)nc3oc4ccccc4c23)cc1]

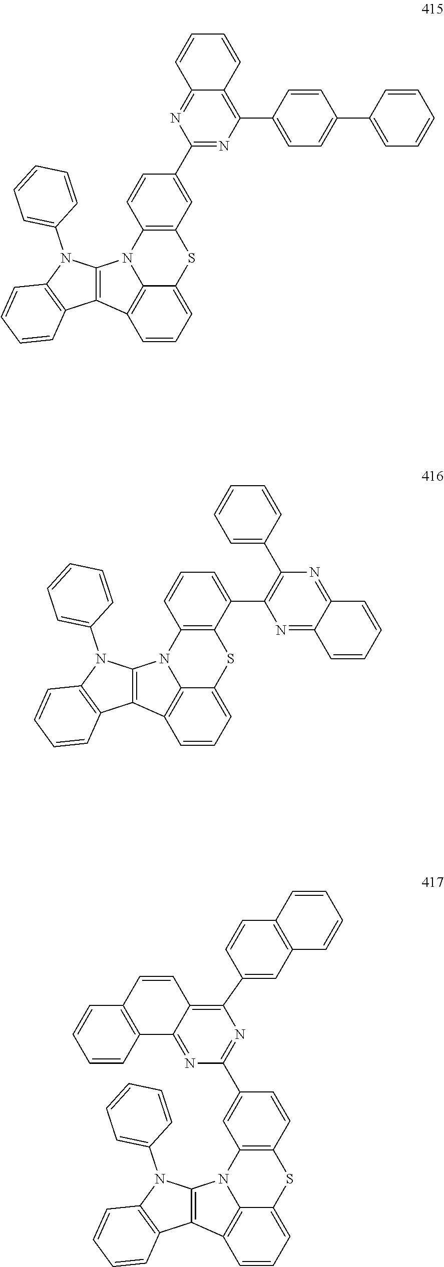 [SMILES: c1ccc(-c2ccc(-c3nc(-c4ccc5c(c4)Sc4cccc6c7c8ccccc8n(-c8ccccc8)c7n-5c46)nc4ccccc34)cc2)cc1 ; c1ccc(-c2nc3ccccc3nc2-c2cccc3c2Sc2cccc4c5c6ccccc6n(-c6ccccc6)c5n-3c24)cc1 ; c1ccc(-n2c3ccccc3c3c4cccc5c4n(c32)-c2cc(-c3nc(-c4ccc6ccccc6c4)c4ccc6ccccc6c4n3)ccc2S5)cc1]

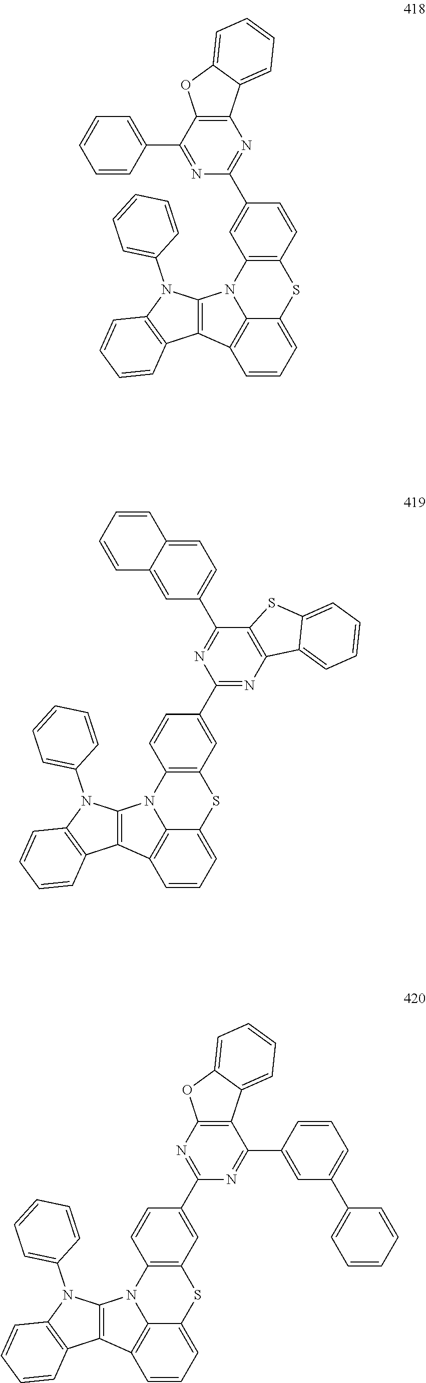 [SMILES: c1ccc(-c2nc(-c3ccc4c(c3)-n3c5c(cccc5c5c6ccccc6n(-c6ccccc6)c53)S4)nc3c2oc2ccccc23)cc1 ; c1ccc(-n2c3ccccc3c3c4cccc5c4n(c32)-c2ccc(-c3nc(-c4ccc6ccccc6c4)c4sc6ccccc6c4n3)cc2S5)cc1 ; c1ccc(-c2cccc(-c3nc(-c4ccc5c(c4)Sc4cccc6c7c8ccccc8n(-c8ccccc8)c7n-5c46)nc4oc5ccccc5c34)c2)cc1]

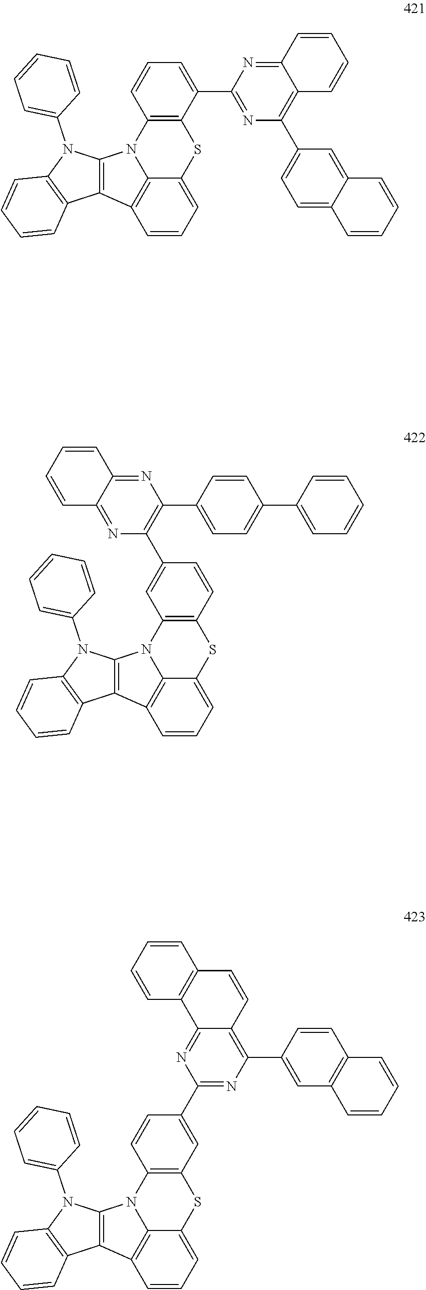 [SMILES: c1ccc(-n2c3ccccc3c3c4cccc5c4n(c32)-c2cccc(-c3nc(-c4ccc6ccccc6c4)c4ccccc4n3)c2S5)cc1 ; c1ccc(-c2ccc(-c3nc4ccccc4nc3-c3ccc4c(c3)-n3c5c(cccc5c5c6ccccc6n(-c6ccccc6)c53)S4)cc2)cc1 ; c1ccc(-n2c3ccccc3c3c4cccc5c4n(c32)-c2ccc(-c3nc(-c4ccc6ccccc6c4)c4ccc6ccccc6c4n3)cc2S5)cc1]

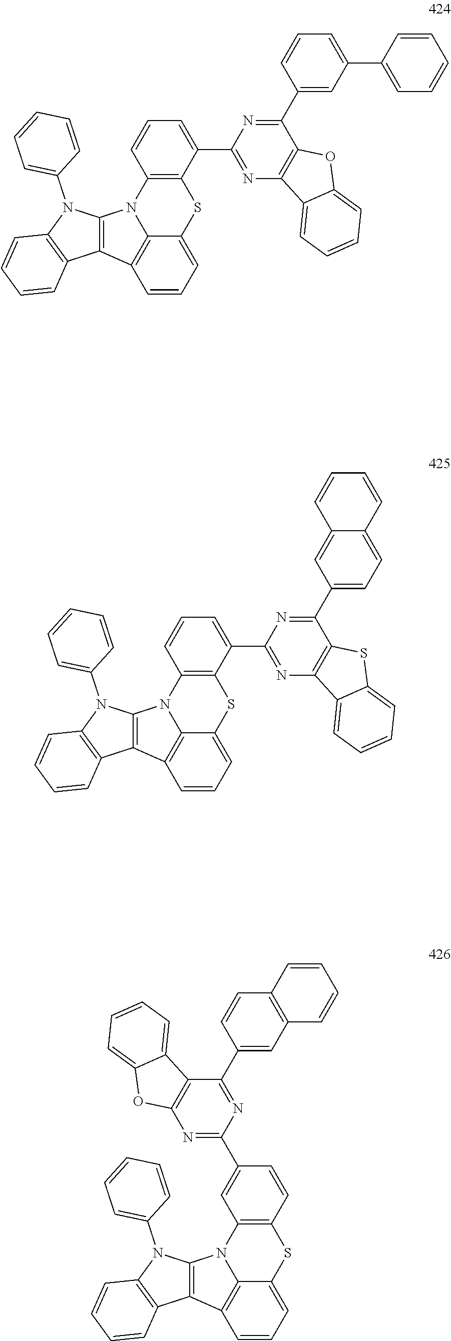 [SMILES: c1ccc(-c2cccc(-c3nc(-c4cccc5c4Sc4cccc6c7c8ccccc8n(-c8ccccc8)c7n-5c46)nc4c3oc3ccccc34)c2)cc1 ; c1ccc(-n2c3ccccc3c3c4cccc5c4n(c32)-c2cccc(-c3nc(-c4ccc6ccccc6c4)c4sc6ccccc6c4n3)c2S5)cc1 ; c1ccc(-n2c3ccccc3c3c4cccc5c4n(c32)-c2cc(-c3nc(-c4ccc6ccccc6c4)c4c(n3)oc3ccccc34)ccc2S5)cc1]

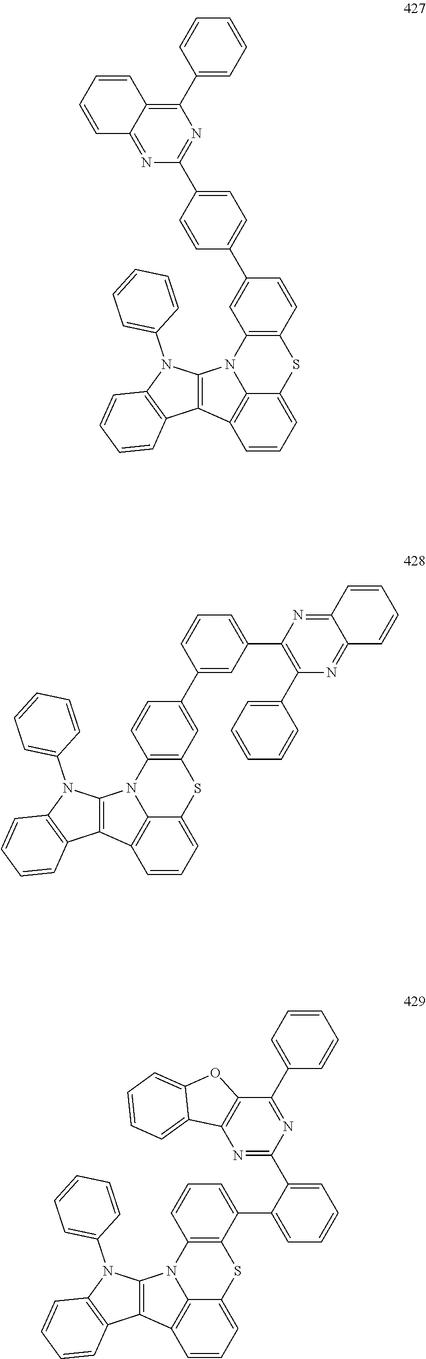 [SMILES: c1ccc(-c2nc(-c3ccc(-c4ccc5c(c4)-n4c6c(cccc6c6c7ccccc7n(-c7ccccc7)c64)S5)cc3)nc3ccccc23)cc1 ; c1ccc(-c2nc3ccccc3nc2-c2cccc(-c3ccc4c(c3)Sc3cccc5c6c7ccccc7n(-c7ccccc7)c6n-4c35)c2)cc1 ; c1ccc(-c2nc(-c3ccccc3-c3cccc4c3Sc3cccc5c6c7ccccc7n(-c7ccccc7)c6n-4c35)nc3c2oc2ccccc23)cc1]

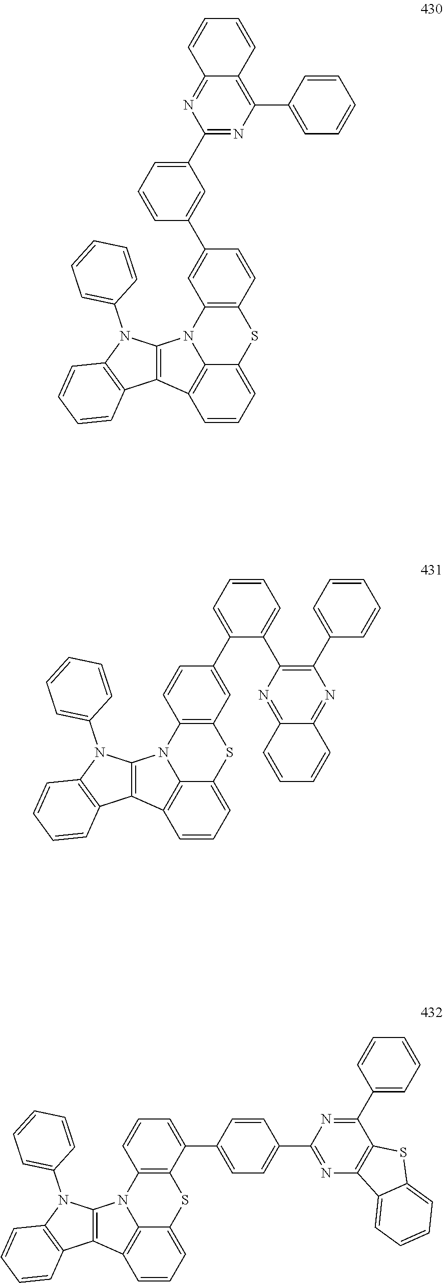 [SMILES: c1ccc(-c2nc(-c3cccc(-c4ccc5c(c4)-n4c6c(cccc6c6c7ccccc7n(-c7ccccc7)c64)S5)c3)nc3ccccc23)cc1 ; c1ccc(-c2nc3ccccc3nc2-c2ccccc2-c2ccc3c(c2)Sc2cccc4c5c6ccccc6n(-c6ccccc6)c5n-3c24)cc1 ; c1ccc(-c2nc(-c3ccc(-c4cccc5c4Sc4cccc6c7c8ccccc8n(-c8ccccc8)c7n-5c46)cc3)nc3c2sc2ccccc23)cc1]

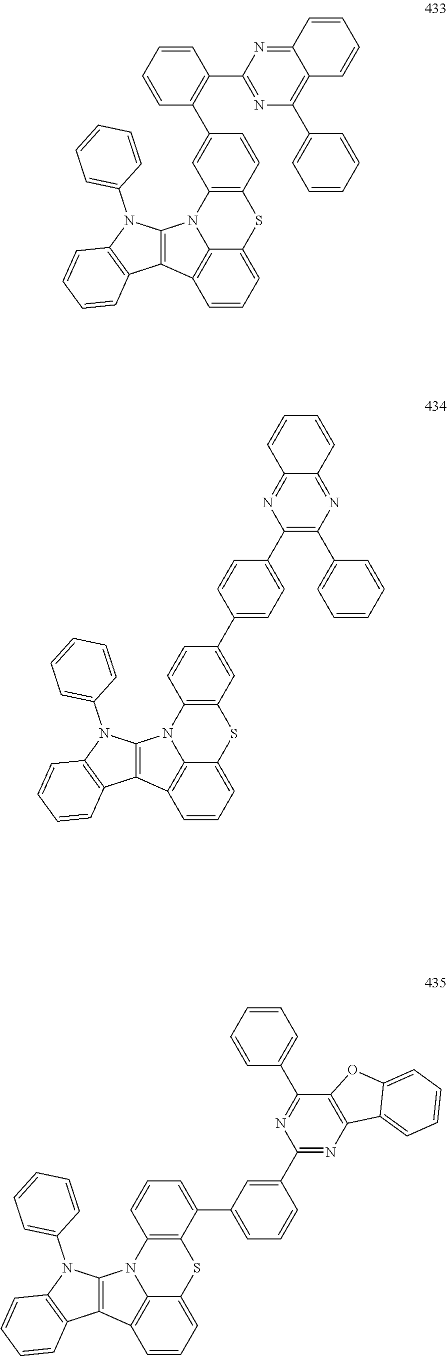 [SMILES: c1ccc(-c2nc(-c3ccccc3-c3ccc4c(c3)-n3c5c(cccc5c5c6ccccc6n(-c6ccccc6)c53)S4)nc3ccccc23)cc1 ; c1ccc(-c2nc3ccccc3nc2-c2ccc(-c3ccc4c(c3)Sc3cccc5c6c7ccccc7n(-c7ccccc7)c6n-4c35)cc2)cc1 ; c1ccc(-c2nc(-c3cccc(-c4cccc5c4Sc4cccc6c7c8ccccc8n(-c8ccccc8)c7n-5c46)c3)nc3c2oc2ccccc23)cc1]

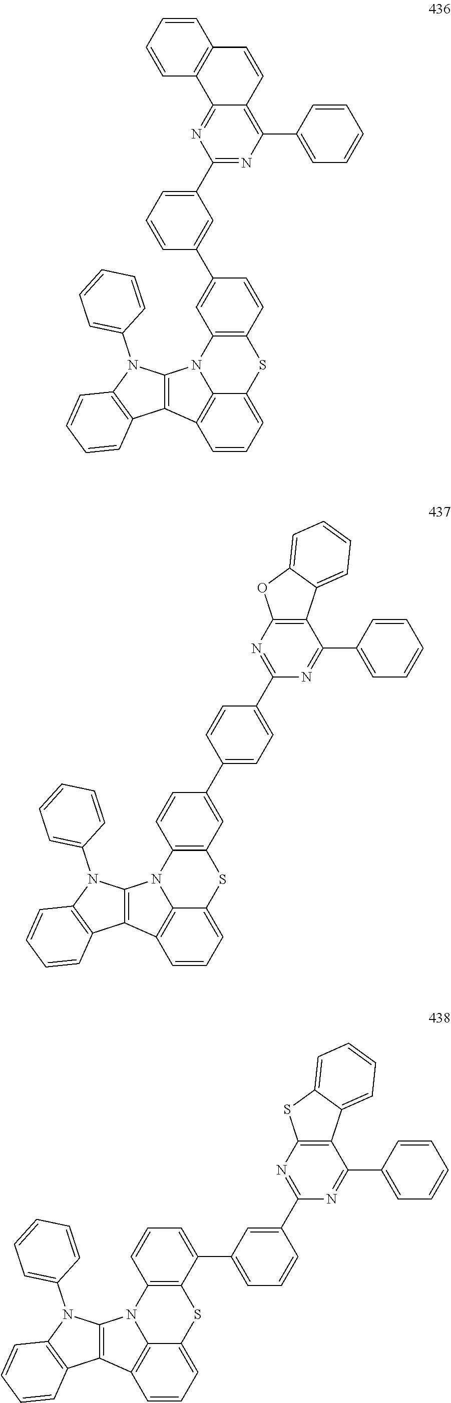 [SMILES: c1ccc(-c2nc(-c3cccc(-c4ccc5c(c4)-n4c6c(cccc6c6c7ccccc7n(-c7ccccc7)c64)S5)c3)nc3c2ccc2ccccc23)cc1 ; c1ccc(-c2nc(-c3ccc(-c4ccc5c(c4)Sc4cccc6c7c8ccccc8n(-c8ccccc8)c7n-5c46)cc3)nc3oc4ccccc4c23)cc1 ; c1ccc(-c2nc(-c3cccc(-c4cccc5c4Sc4cccc6c7c8ccccc8n(-c8ccccc8)c7n-5c46)c3)nc3sc4ccccc4c23)cc1]

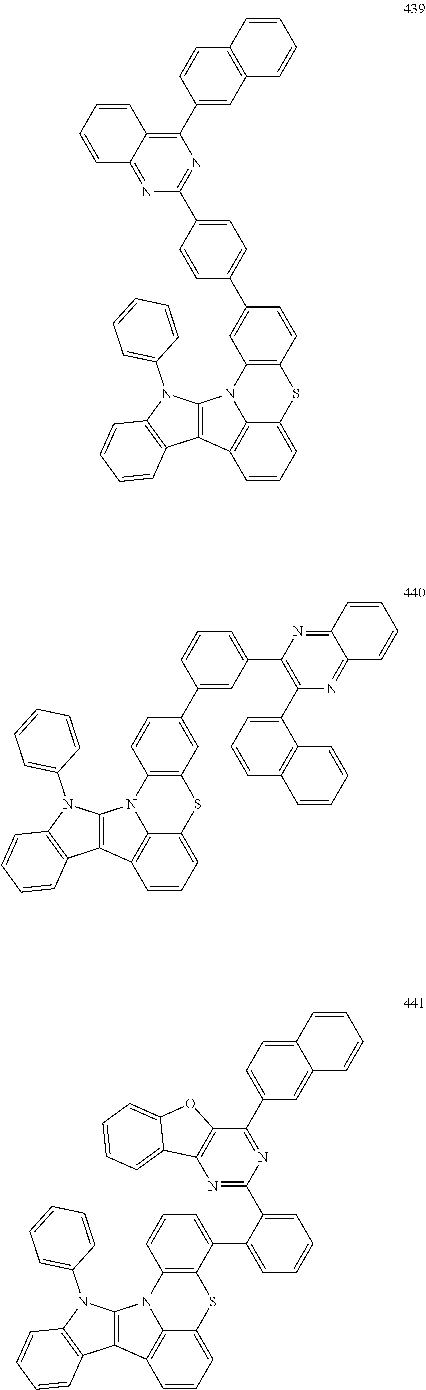 [SMILES: c1ccc(-n2c3ccccc3c3c4cccc5c4n(c32)-c2cc(-c3ccc(-c4nc(-c6ccc7ccccc7c6)c6ccccc6n4)cc3)ccc2S5)cc1 ; c1ccc(-n2c3ccccc3c3c4cccc5c4n(c32)-c2ccc(-c3cccc(-c4nc6ccccc6nc4-c4cccc6ccccc46)c3)cc2S5)cc1 ; c1ccc(-n2c3ccccc3c3c4cccc5c4n(c32)-c2cccc(-c3ccccc3-c3nc(-c4ccc6ccccc6c4)c4oc6ccccc6c4n3)c2S5)cc1]

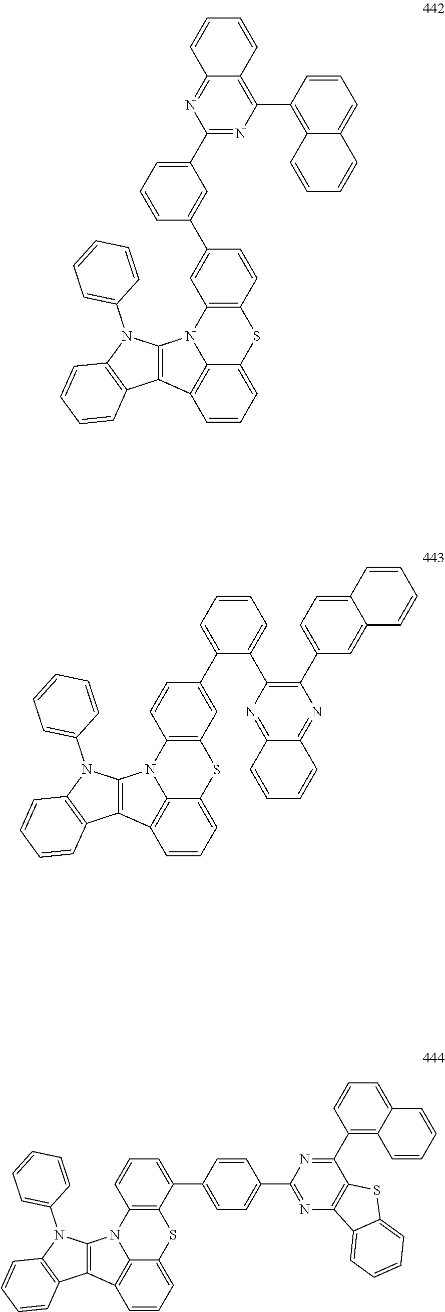 [SMILES: c1ccc(-n2c3ccccc3c3c4cccc5c4n(c32)-c2cc(-c3cccc(-c4nc(-c6cccc7ccccc67)c6ccccc6n4)c3)ccc2S5)cc1 ; c1ccc(-n2c3ccccc3c3c4cccc5c4n(c32)-c2ccc(-c3ccccc3-c3nc4ccccc4nc3-c3ccc4ccccc4c3)cc2S5)cc1 ; c1ccc(-n2c3ccccc3c3c4cccc5c4n(c32)-c2cccc(-c3ccc(-c4nc(-c6cccc7ccccc67)c6sc7ccccc7c6n4)cc3)c2S5)cc1]

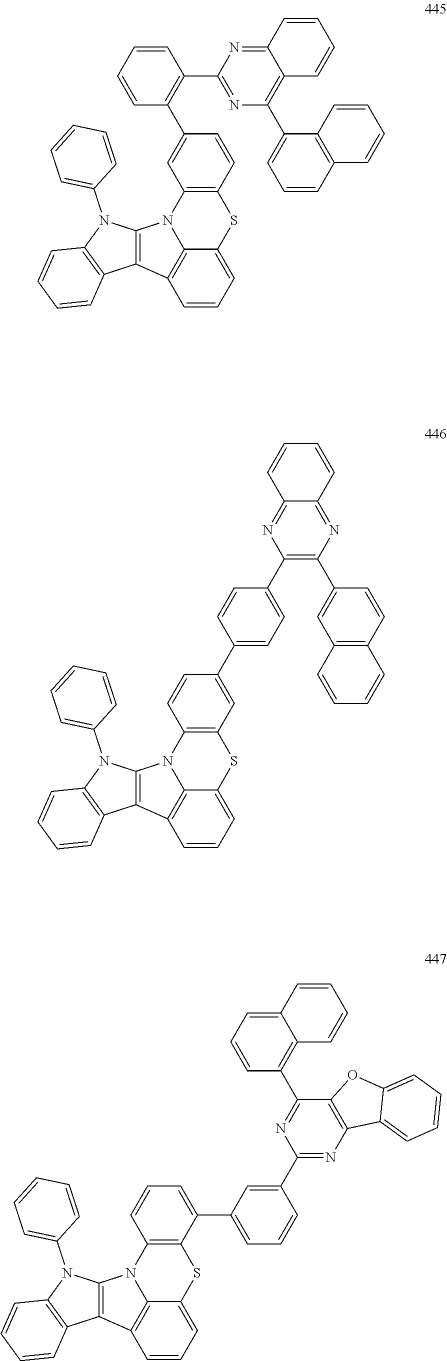 [SMILES: c1ccc(-n2c3ccccc3c3c4cccc5c4n(c32)-c2cc(-c3ccccc3-c3nc(-c4cccc6ccccc46)c4ccccc4n3)ccc2S5)cc1 ; c1ccc(-n2c3ccccc3c3c4cccc5c4n(c32)-c2ccc(-c3ccc(-c4nc6ccccc6nc4-c4ccc6ccccc6c4)cc3)cc2S5)cc1 ; c1ccc(-n2c3ccccc3c3c4cccc5c4n(c32)-c2cccc(-c3cccc(-c4nc(-c6cccc7ccccc67)c6oc7ccccc7c6n4)c3)c2S5)cc1]

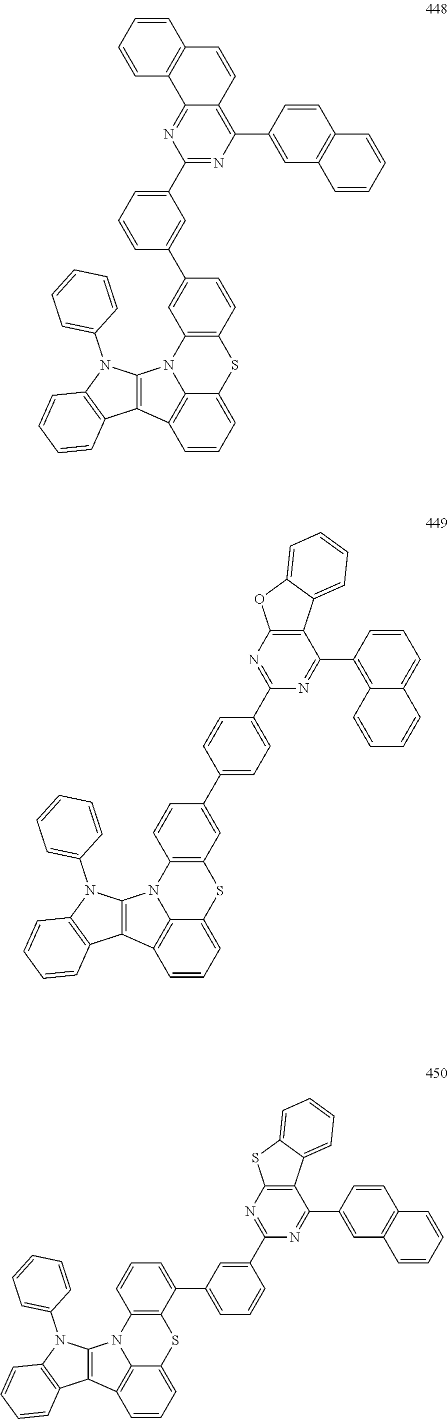 [SMILES: c1ccc(-n2c3ccccc3c3c4cccc5c4n(c32)-c2cc(-c3cccc(-c4nc(-c6ccc7ccccc7c6)c6ccc7ccccc7c6n4)c3)ccc2S5)cc1 ; c1ccc(-n2c3ccccc3c3c4cccc5c4n(c32)-c2ccc(-c3ccc(-c4nc(-c6cccc7ccccc67)c6c(n4)oc4ccccc46)cc3)cc2S5)cc1 ; c1ccc(-n2c3ccccc3c3c4cccc5c4n(c32)-c2cccc(-c3cccc(-c4nc(-c6ccc7ccccc7c6)c6c(n4)sc4ccccc46)c3)c2S5)cc1]

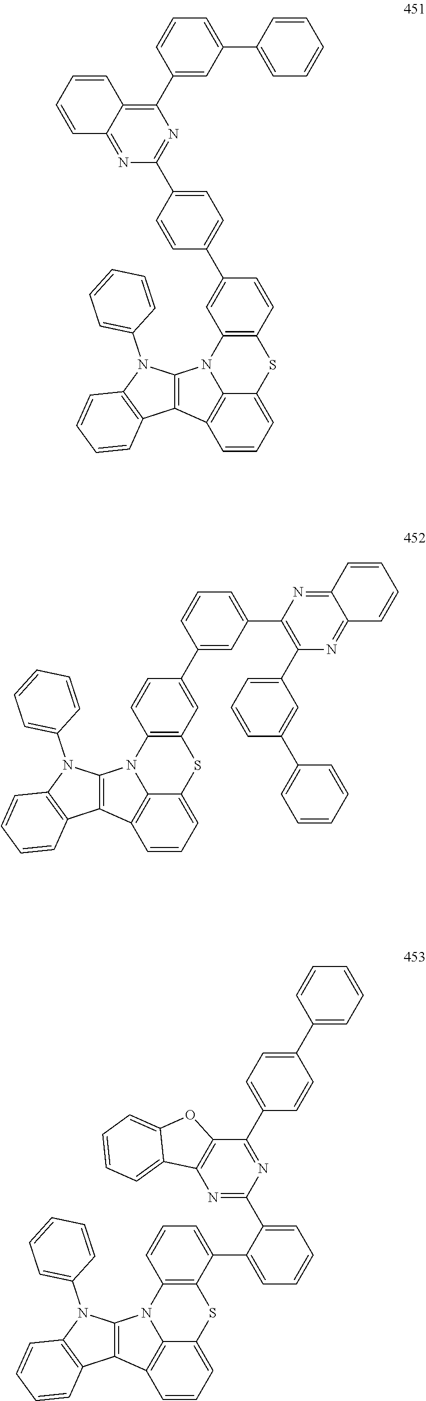 [SMILES: c1ccc(-c2cccc(-c3nc(-c4ccc(-c5ccc6c(c5)-n5c7c(cccc7c7c8ccccc8n(-c8ccccc8)c75)S6)cc4)nc4ccccc34)c2)cc1 ; c1ccc(-c2cccc(-c3nc4ccccc4nc3-c3cccc(-c4ccc5c(c4)Sc4cccc6c7c8ccccc8n(-c8ccccc8)c7n-5c46)c3)c2)cc1 ; c1ccc(-c2ccc(-c3nc(-c4ccccc4-c4cccc5c4Sc4cccc6c7c8ccccc8n(-c8ccccc8)c7n-5c46)nc4c3oc3ccccc34)cc2)cc1]

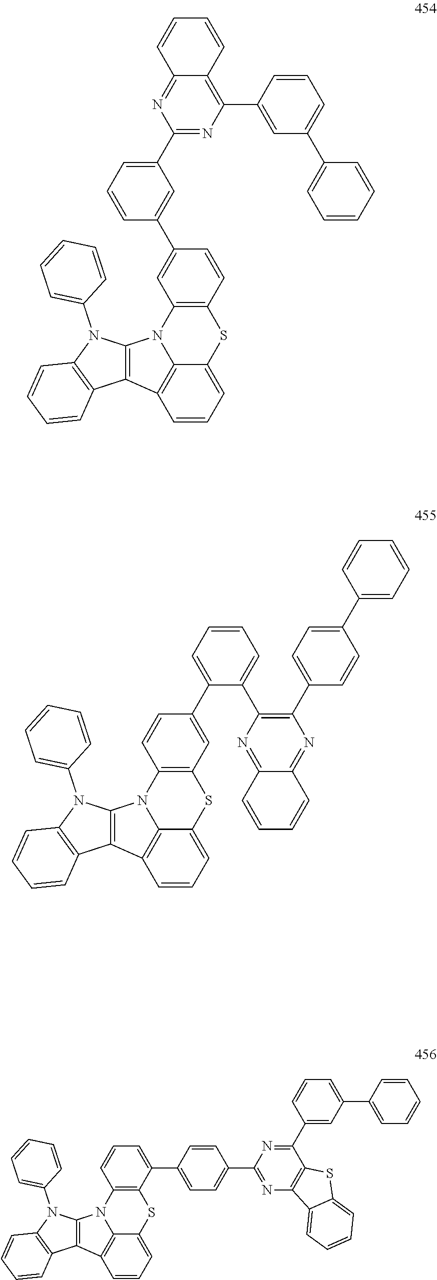 [SMILES: c1ccc(-c2cccc(-c3nc(-c4cccc(-c5ccc6c(c5)-n5c7c(cccc7c7c8ccccc8n(-c8ccccc8)c75)S6)c4)nc4ccccc34)c2)cc1 ; c1ccc(-c2ccc(-c3nc4ccccc4nc3-c3ccccc3-c3ccc4c(c3)Sc3cccc5c6c7ccccc7n(-c7ccccc7)c6n-4c35)cc2)cc1 ; c1ccc(-c2cccc(-c3nc(-c4ccc(-c5cccc6c5Sc5cccc7c8c9ccccc9n(-c9ccccc9)c8n-6c57)cc4)nc4c3sc3ccccc34)c2)cc1]

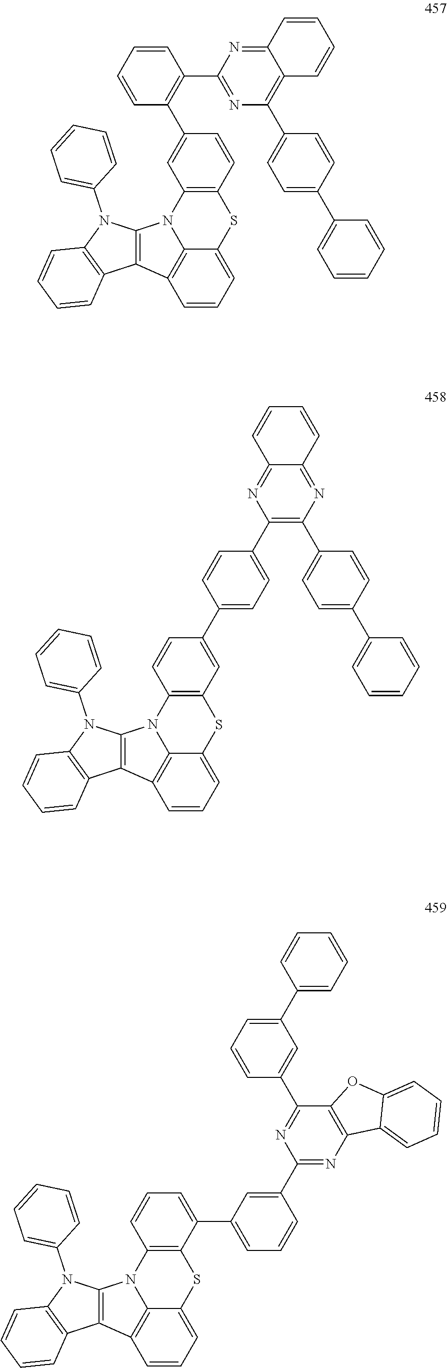 [SMILES: c1ccc(-c2ccc(-c3nc(-c4ccccc4-c4ccc5c(c4)-n4c6c(cccc6c6c7ccccc7n(-c7ccccc7)c64)S5)nc4ccccc34)cc2)cc1 ; c1ccc(-c2ccc(-c3nc4ccccc4nc3-c3ccc(-c4ccc5c(c4)Sc4cccc6c7c8ccccc8n(-c8ccccc8)c7n-5c46)cc3)cc2)cc1 ; c1ccc(-c2cccc(-c3nc(-c4cccc(-c5cccc6c5Sc5cccc7c8c9ccccc9n(-c9ccccc9)c8n-6c57)c4)nc4c3oc3ccccc34)c2)cc1]

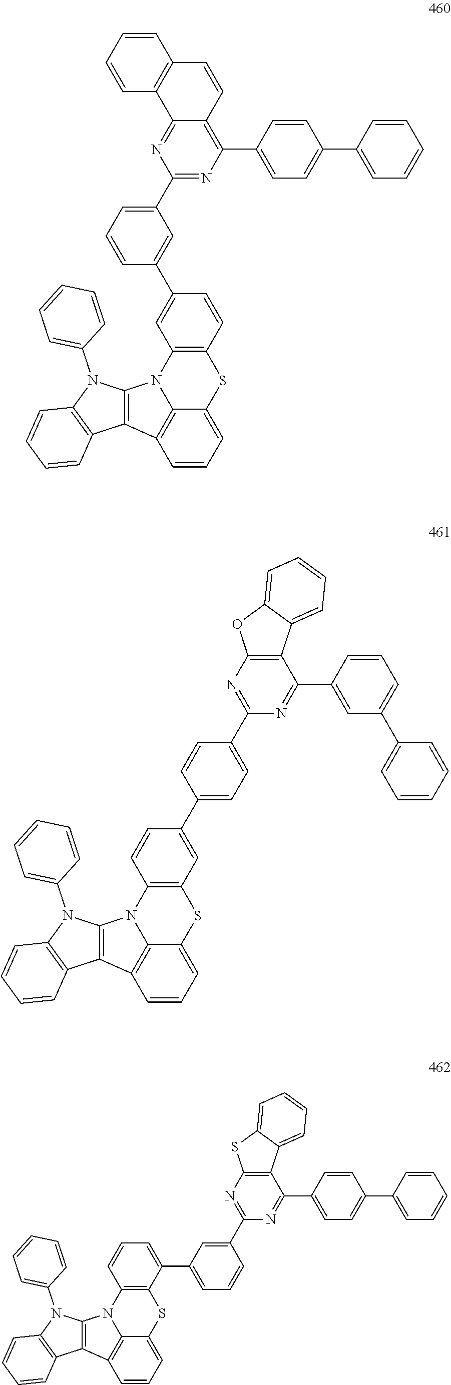 [SMILES: c1ccc(-c2ccc(-c3nc(-c4cccc(-c5ccc6c(c5)-n5c7c(cccc7c7c8ccccc8n(-c8ccccc8)c75)S6)c4)nc4c3ccc3ccccc34)cc2)cc1 ; c1ccc(-c2cccc(-c3nc(-c4ccc(-c5ccc6c(c5)Sc5cccc7c8c9ccccc9n(-c9ccccc9)c8n-6c57)cc4)nc4oc5ccccc5c34)c2)cc1 ; c1ccc(-c2ccc(-c3nc(-c4cccc(-c5cccc6c5Sc5cccc7c8c9ccccc9n(-c9ccccc9)c8n-6c57)c4)nc4sc5ccccc5c34)cc2)cc1]

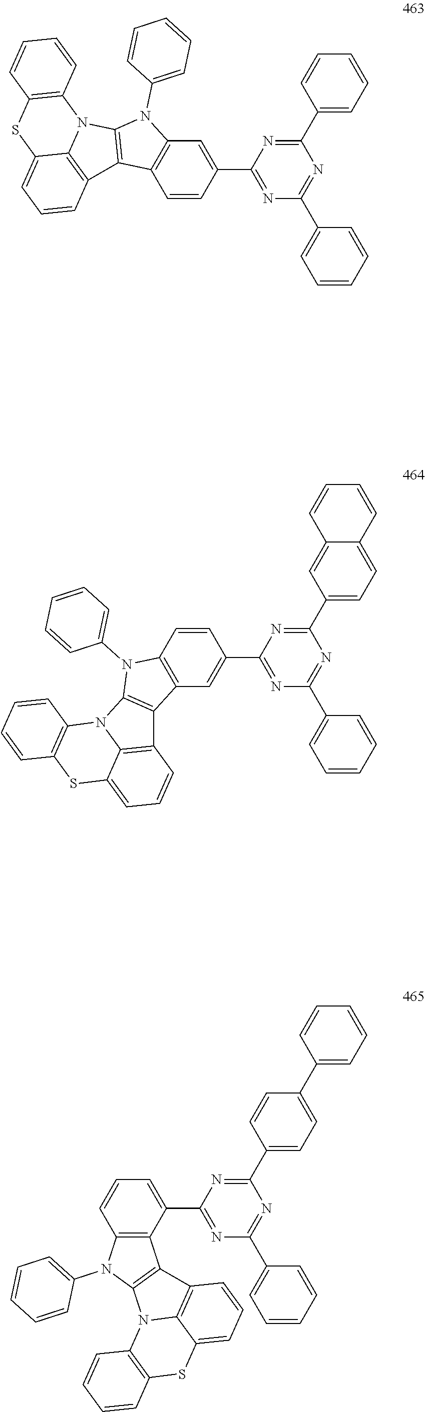 [SMILES: c1ccc(-c2nc(-c3ccccc3)nc(-c3ccc4c5c6cccc7c6n(c5n(-c5ccccc5)c4c3)-c3ccccc3S7)n2)cc1 ; c1ccc(-c2nc(-c3ccc4ccccc4c3)nc(-c3ccc4c(c3)c3c5cccc6c5n(c3n4-c3ccccc3)-c3ccccc3S6)n2)cc1 ; c1ccc(-c2ccc(-c3nc(-c4ccccc4)nc(-c4cccc5c4c4c6cccc7c6n(c4n5-c4ccccc4)-c4ccccc4S7)n3)cc2)cc1]

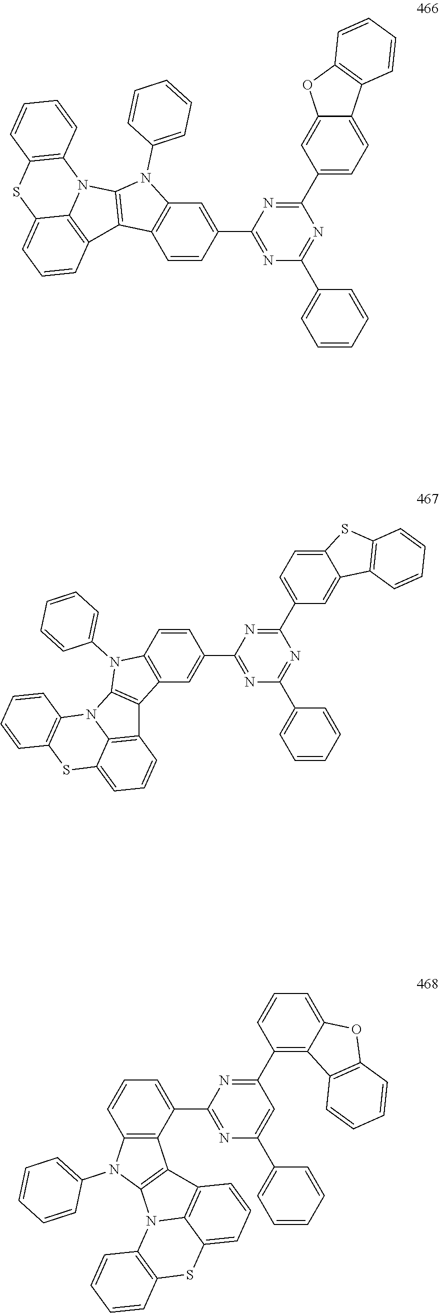 [SMILES: c1ccc(-c2nc(-c3ccc4c(c3)oc3ccccc34)nc(-c3ccc4c5c6cccc7c6n(c5n(-c5ccccc5)c4c3)-c3ccccc3S7)n2)cc1 ; c1ccc(-c2nc(-c3ccc4sc5ccccc5c4c3)nc(-c3ccc4c(c3)c3c5cccc6c5n(c3n4-c3ccccc3)-c3ccccc3S6)n2)cc1 ; c1ccc(-c2cc(-c3cccc4oc5ccccc5c34)nc(-c3cccc4c3c3c5cccc6c5n(c3n4-c3ccccc3)-c3ccccc3S6)n2)cc1]

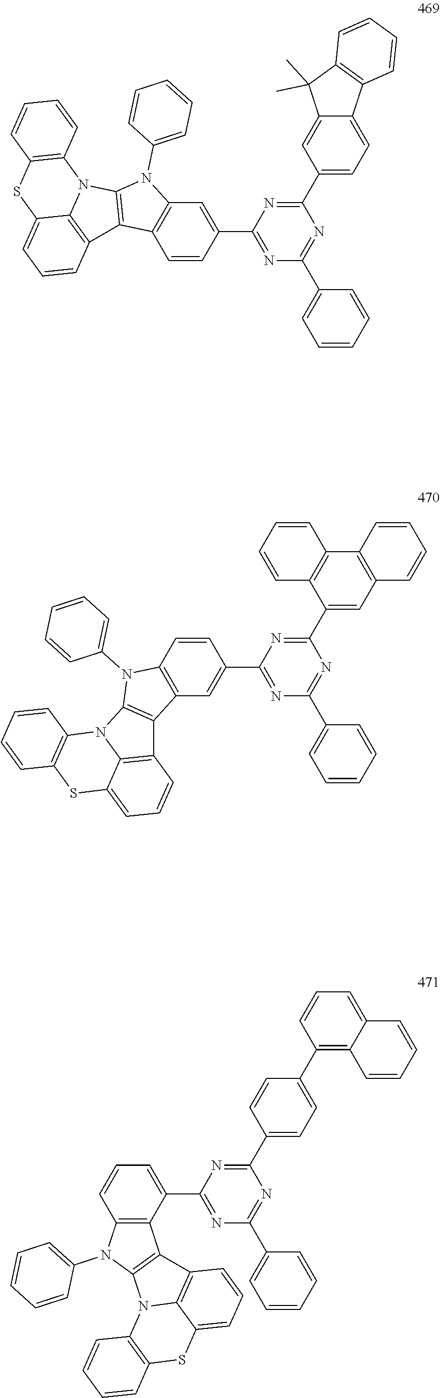 [SMILES: CC1(C)c2ccccc2-c2ccc(-c3nc(-c4ccccc4)nc(-c4ccc5c6c7cccc8c7n(c6n(-c6ccccc6)c5c4)-c4ccccc4S8)n3)cc21 ; c1ccc(-c2nc(-c3ccc4c(c3)c3c5cccc6c5n(c3n4-c3ccccc3)-c3ccccc3S6)nc(-c3cc4ccccc4c4ccccc34)n2)cc1 ; c1ccc(-c2nc(-c3ccc(-c4cccc5ccccc45)cc3)nc(-c3cccc4c3c3c5cccc6c5n(c3n4-c3ccccc3)-c3ccccc3S6)n2)cc1]

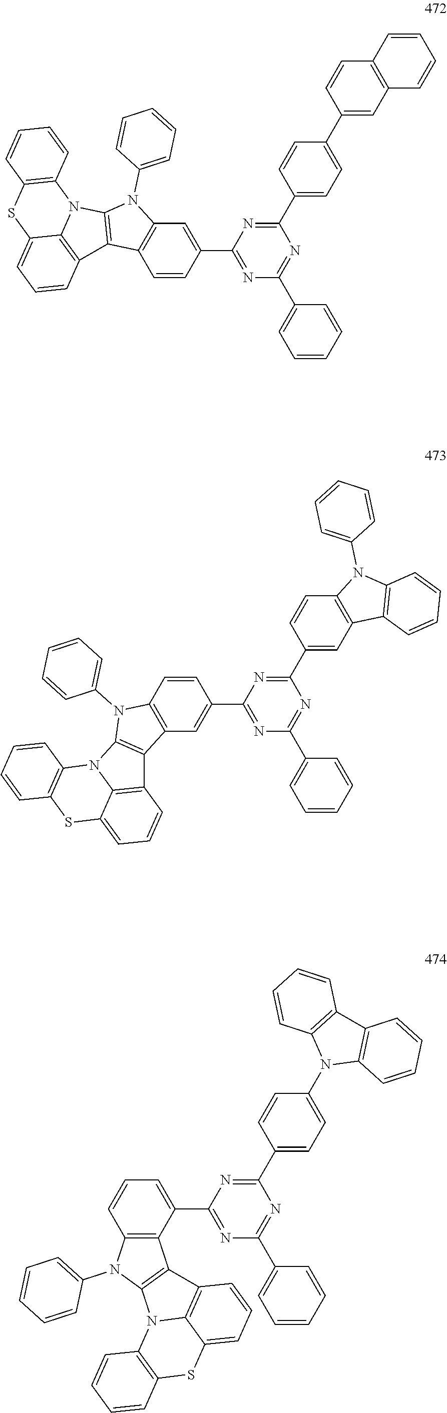 [SMILES: c1ccc(-c2nc(-c3ccc(-c4ccc5ccccc5c4)cc3)nc(-c3ccc4c5c6cccc7c6n(c5n(-c5ccccc5)c4c3)-c3ccccc3S7)n2)cc1 ; c1ccc(-c2nc(-c3ccc4c(c3)c3ccccc3n4-c3ccccc3)nc(-c3ccc4c(c3)c3c5cccc6c5n(c3n4-c3ccccc3)-c3ccccc3S6)n2)cc1 ; c1ccc(-c2nc(-c3ccc(-n4c5ccccc5c5ccccc54)cc3)nc(-c3cccc4c3c3c5cccc6c5n(c3n4-c3ccccc3)-c3ccccc3S6)n2)cc1]

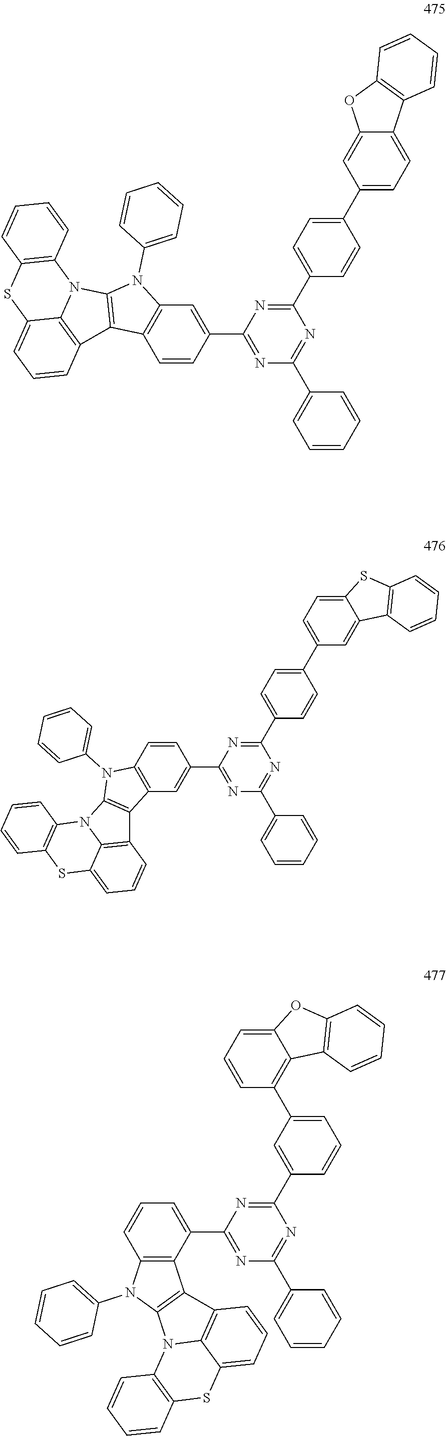 [SMILES: c1ccc(-c2nc(-c3ccc(-c4ccc5c(c4)oc4ccccc45)cc3)nc(-c3ccc4c5c6cccc7c6n(c5n(-c5ccccc5)c4c3)-c3ccccc3S7)n2)cc1 ; c1ccc(-c2nc(-c3ccc(-c4ccc5sc6ccccc6c5c4)cc3)nc(-c3ccc4c(c3)c3c5cccc6c5n(c3n4-c3ccccc3)-c3ccccc3S6)n2)cc1 ; c1ccc(-c2nc(-c3cccc(-c4cccc5oc6ccccc6c45)c3)nc(-c3cccc4c3c3c5cccc6c5n(c3n4-c3ccccc3)-c3ccccc3S6)n2)cc1]

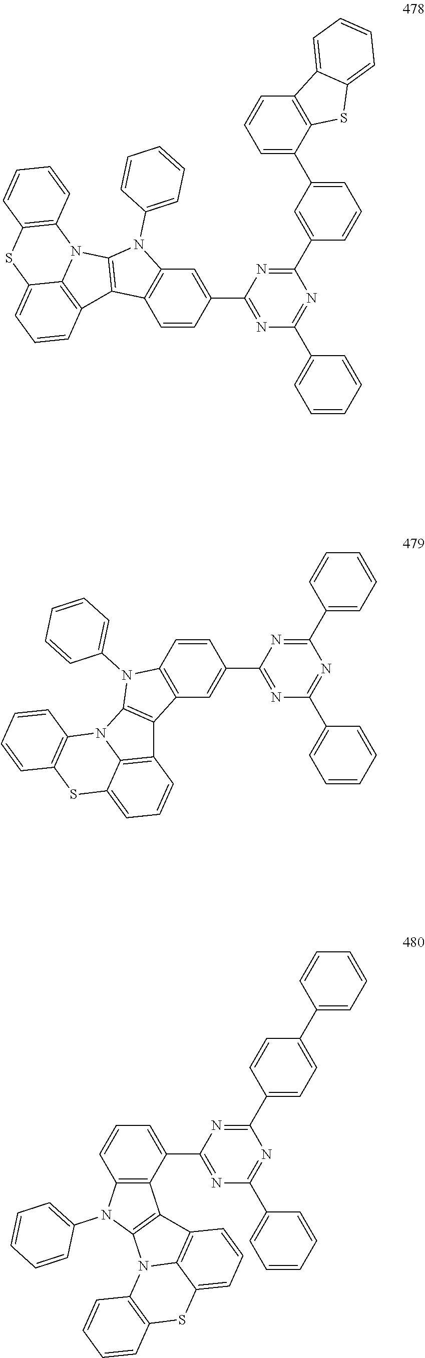 [SMILES: c1ccc(-c2nc(-c3cccc(-c4cccc5c4sc4ccccc45)c3)nc(-c3ccc4c5c6cccc7c6n(c5n(-c5ccccc5)c4c3)-c3ccccc3S7)n2)cc1 ; c1ccc(-c2nc(-c3ccccc3)nc(-c3ccc4c(c3)c3c5cccc6c5n(c3n4-c3ccccc3)-c3ccccc3S6)n2)cc1 ; c1ccc(-c2ccc(-c3nc(-c4ccccc4)nc(-c4cccc5c4c4c6cccc7c6n(c4n5-c4ccccc4)-c4ccccc4S7)n3)cc2)cc1]

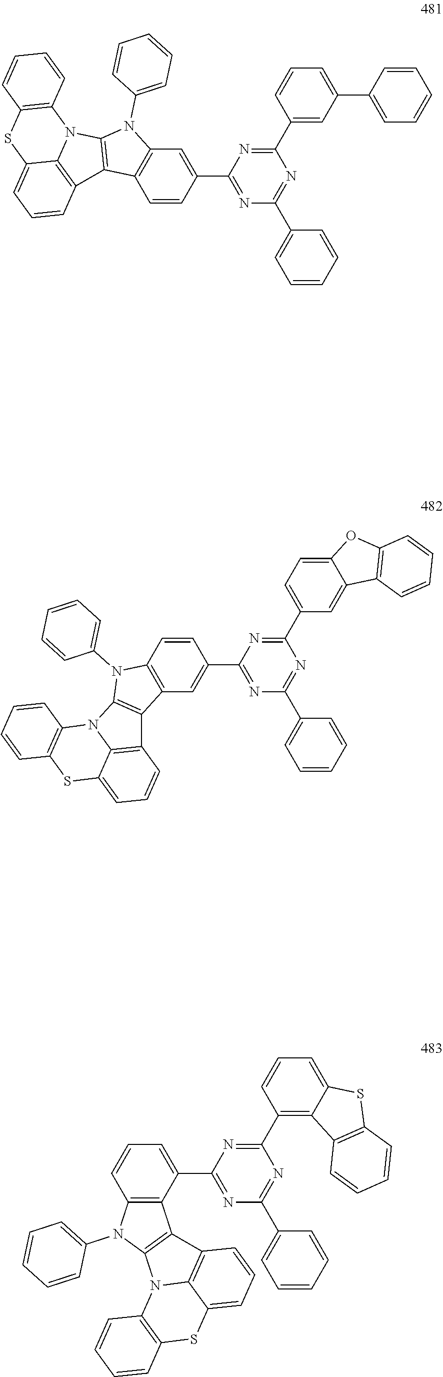 [SMILES: c1ccc(-c2cccc(-c3nc(-c4ccccc4)nc(-c4ccc5c6c7cccc8c7n(c6n(-c6ccccc6)c5c4)-c4ccccc4S8)n3)c2)cc1 ; c1ccc(-c2nc(-c3ccc4oc5ccccc5c4c3)nc(-c3ccc4c(c3)c3c5cccc6c5n(c3n4-c3ccccc3)-c3ccccc3S6)n2)cc1 ; c1ccc(-c2nc(-c3cccc4sc5ccccc5c34)nc(-c3cccc4c3c3c5cccc6c5n(c3n4-c3ccccc3)-c3ccccc3S6)n2)cc1]

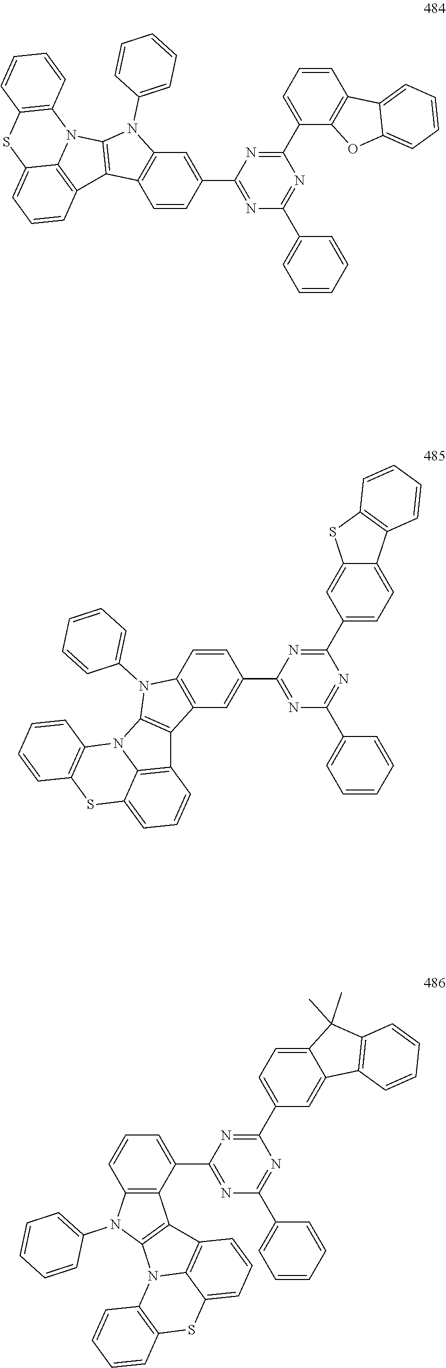 [SMILES: c1ccc(-c2nc(-c3ccc4c5c6cccc7c6n(c5n(-c5ccccc5)c4c3)-c3ccccc3S7)nc(-c3cccc4c3oc3ccccc34)n2)cc1 ; c1ccc(-c2nc(-c3ccc4c(c3)sc3ccccc34)nc(-c3ccc4c(c3)c3c5cccc6c5n(c3n4-c3ccccc3)-c3ccccc3S6)n2)cc1 ; CC1(C)c2ccccc2-c2cc(-c3nc(-c4ccccc4)nc(-c4cccc5c4c4c6cccc7c6n(c4n5-c4ccccc4)-c4ccccc4S7)n3)ccc21]

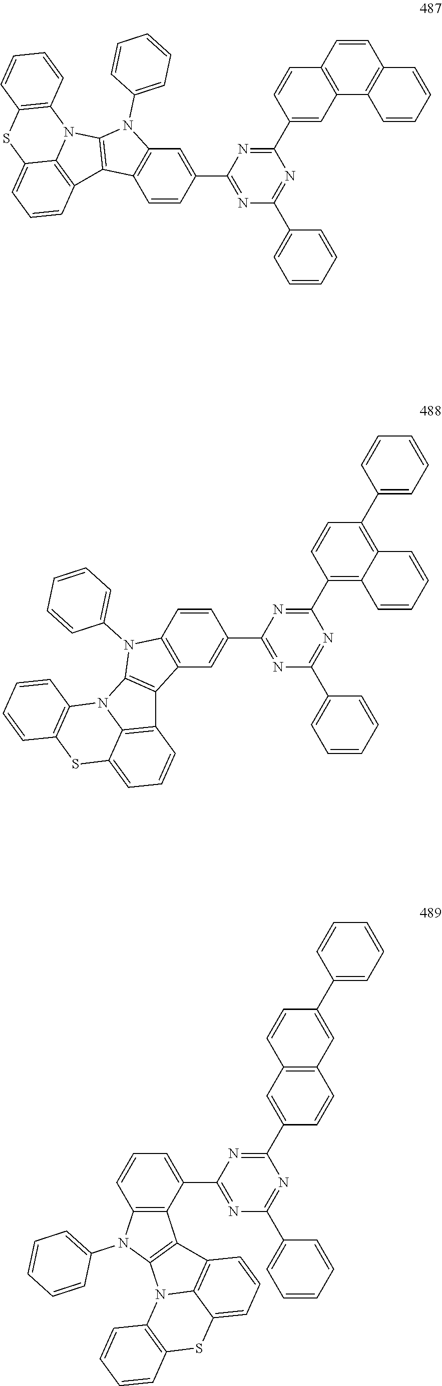 [SMILES: c1ccc(-c2nc(-c3ccc4ccc5ccccc5c4c3)nc(-c3ccc4c5c6cccc7c6n(c5n(-c5ccccc5)c4c3)-c3ccccc3S7)n2)cc1 ; c1ccc(-c2nc(-c3ccc4c(c3)c3c5cccc6c5n(c3n4-c3ccccc3)-c3ccccc3S6)nc(-c3ccc(-c4ccccc4)c4ccccc34)n2)cc1 ; c1ccc(-c2ccc3cc(-c4nc(-c5ccccc5)nc(-c5cccc6c5c5c7cccc8c7n(c5n6-c5ccccc5)-c5ccccc5S8)n4)ccc3c2)cc1]

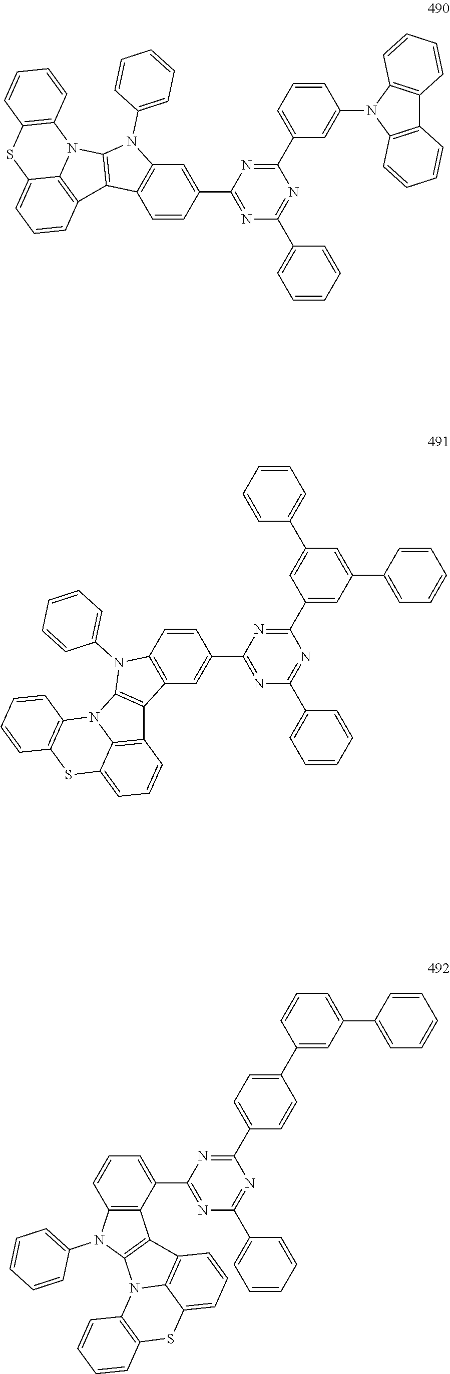 [SMILES: c1ccc(-c2nc(-c3cccc(-n4c5ccccc5c5ccccc54)c3)nc(-c3ccc4c5c6cccc7c6n(c5n(-c5ccccc5)c4c3)-c3ccccc3S7)n2)cc1 ; c1ccc(-c2cc(-c3ccccc3)cc(-c3nc(-c4ccccc4)nc(-c4ccc5c(c4)c4c6cccc7c6n(c4n5-c4ccccc4)-c4ccccc4S7)n3)c2)cc1 ; c1ccc(-c2cccc(-c3ccc(-c4nc(-c5ccccc5)nc(-c5cccc6c5c5c7cccc8c7n(c5n6-c5ccccc5)-c5ccccc5S8)n4)cc3)c2)cc1]

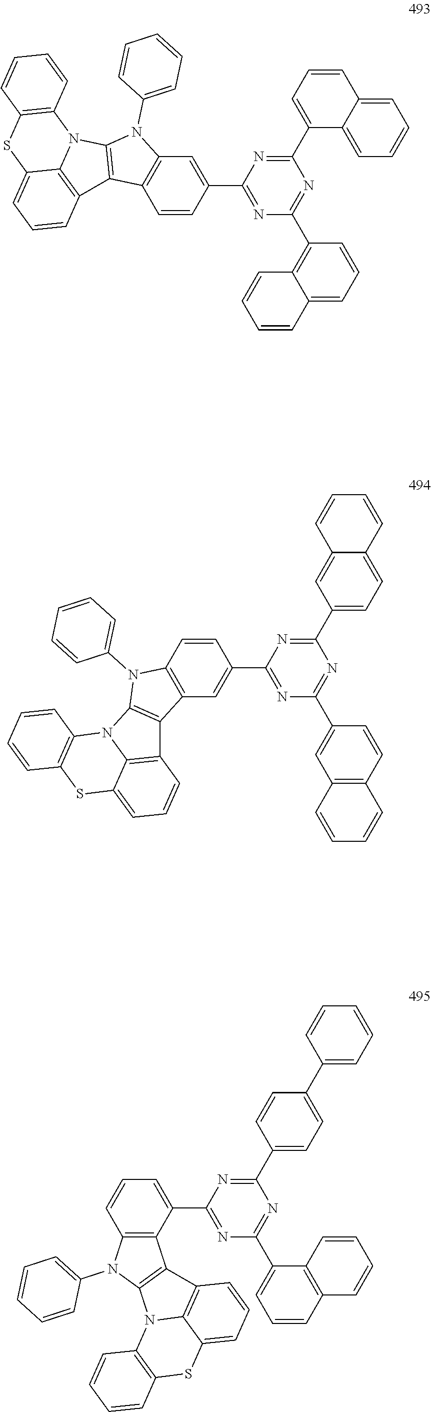 [SMILES: c1ccc(-n2c3cc(-c4nc(-c5cccc6ccccc56)nc(-c5cccc6ccccc56)n4)ccc3c3c4cccc5c4n(c32)-c2ccccc2S5)cc1 ; c1ccc(-n2c3ccc(-c4nc(-c5ccc6ccccc6c5)nc(-c5ccc6ccccc6c5)n4)cc3c3c4cccc5c4n(c32)-c2ccccc2S5)cc1 ; c1ccc(-c2ccc(-c3nc(-c4cccc5ccccc45)nc(-c4cccc5c4c4c6cccc7c6n(c4n5-c4ccccc4)-c4ccccc4S7)n3)cc2)cc1]

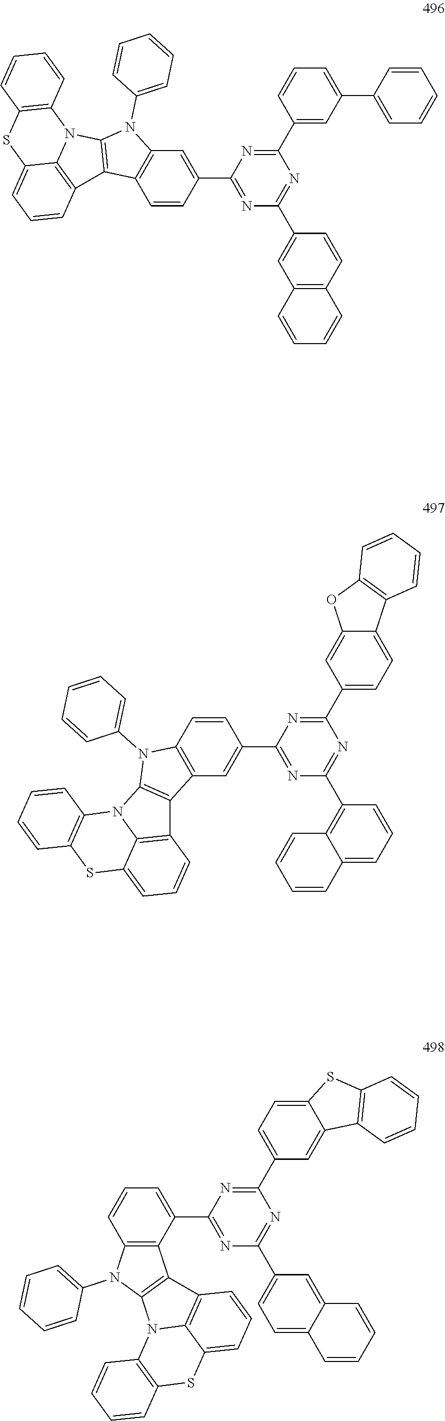 [SMILES: c1ccc(-c2cccc(-c3nc(-c4ccc5ccccc5c4)nc(-c4ccc5c6c7cccc8c7n(c6n(-c6ccccc6)c5c4)-c4ccccc4S8)n3)c2)cc1 ; c1ccc(-n2c3ccc(-c4nc(-c5ccc6c(c5)oc5ccccc56)nc(-c5cccc6ccccc56)n4)cc3c3c4cccc5c4n(c32)-c2ccccc2S5)cc1 ; c1ccc(-n2c3cccc(-c4nc(-c5ccc6ccccc6c5)nc(-c5ccc6sc7ccccc7c6c5)n4)c3c3c4cccc5c4n(c32)-c2ccccc2S5)cc1]

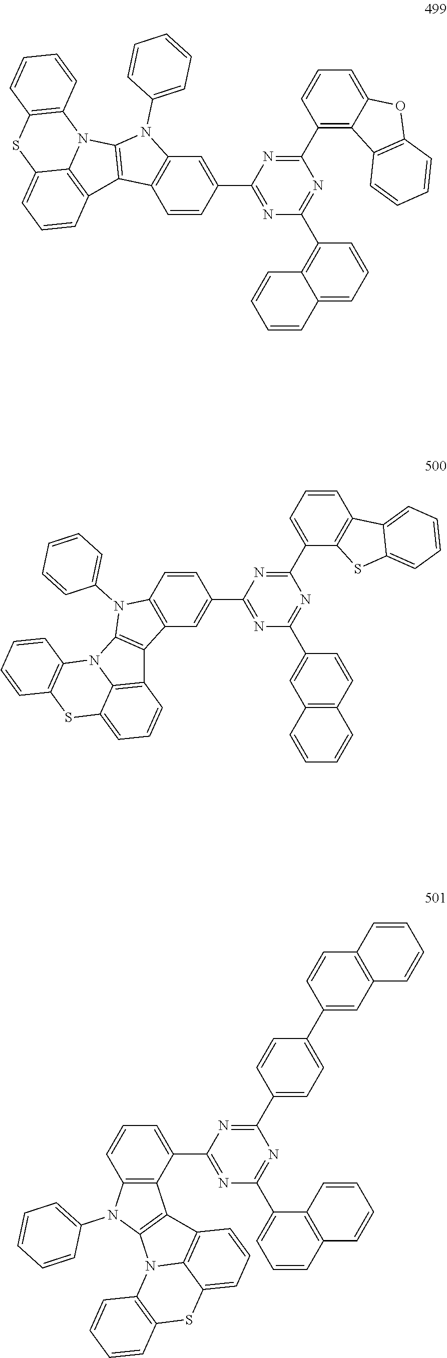 [SMILES: c1ccc(-n2c3cc(-c4nc(-c5cccc6ccccc56)nc(-c5cccc6oc7ccccc7c56)n4)ccc3c3c4cccc5c4n(c32)-c2ccccc2S5)cc1 ; c1ccc(-n2c3ccc(-c4nc(-c5ccc6ccccc6c5)nc(-c5cccc6c5sc5ccccc56)n4)cc3c3c4cccc5c4n(c32)-c2ccccc2S5)cc1 ; c1ccc(-n2c3cccc(-c4nc(-c5ccc(-c6ccc7ccccc7c6)cc5)nc(-c5cccc6ccccc56)n4)c3c3c4cccc5c4n(c32)-c2ccccc2S5)cc1]

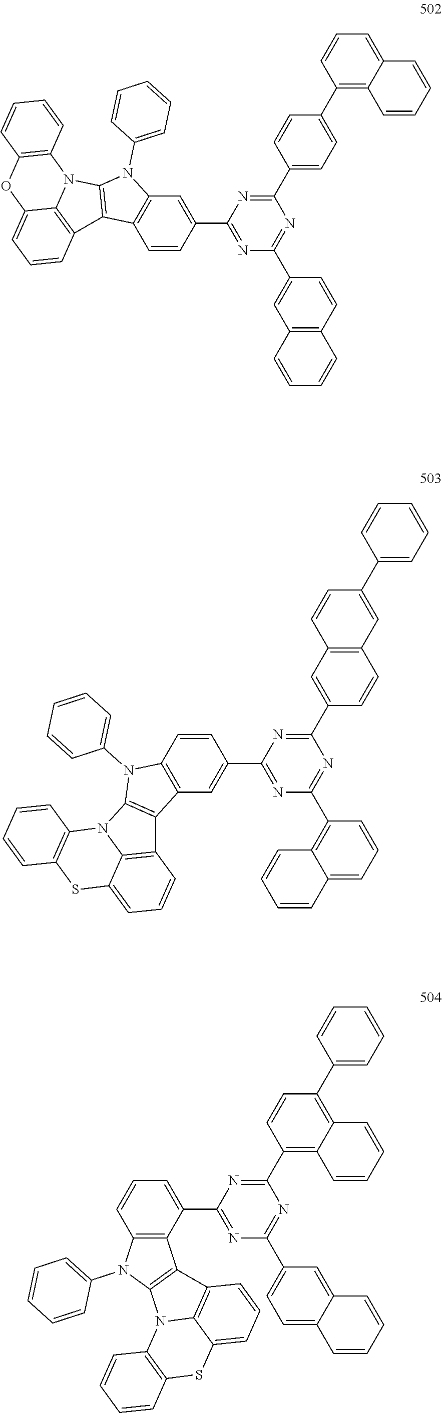 [SMILES: c1ccc(-n2c3cc(-c4nc(-c5ccc(-c6cccc7ccccc67)cc5)nc(-c5ccc6ccccc6c5)n4)ccc3c3c4cccc5c4n(c32)-c2ccccc2O5)cc1 ; c1ccc(-c2ccc3cc(-c4nc(-c5ccc6c(c5)c5c7cccc8c7n(c5n6-c5ccccc5)-c5ccccc5S8)nc(-c5cccc6ccccc56)n4)ccc3c2)cc1 ; c1ccc(-c2ccc(-c3nc(-c4ccc5ccccc5c4)nc(-c4cccc5c4c4c6cccc7c6n(c4n5-c4ccccc4)-c4ccccc4S7)n3)c3ccccc23)cc1]

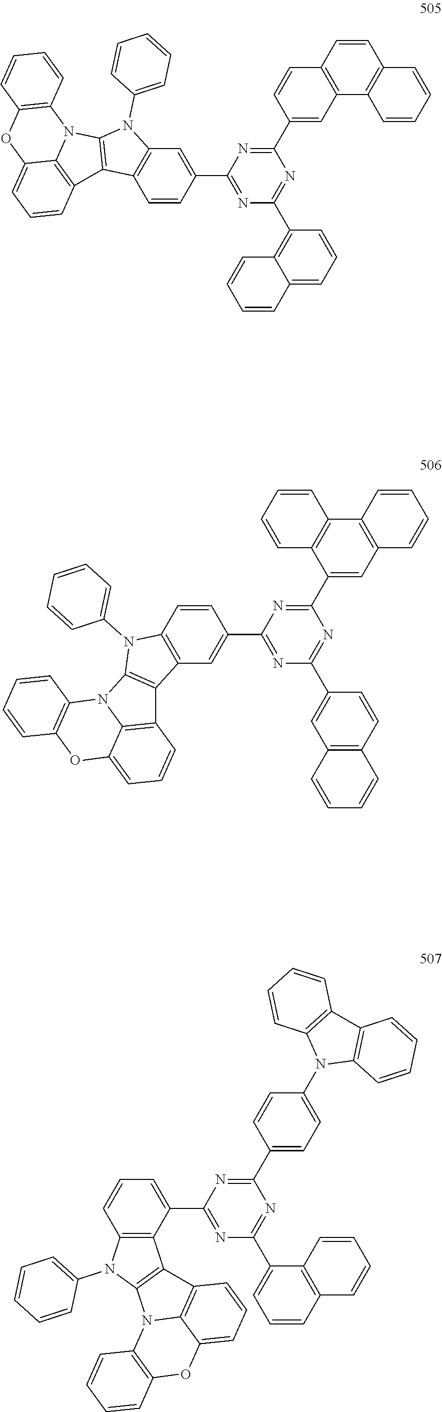 [SMILES: c1ccc(-n2c3cc(-c4nc(-c5ccc6ccc7ccccc7c6c5)nc(-c5cccc6ccccc56)n4)ccc3c3c4cccc5c4n(c32)-c2ccccc2O5)cc1 ; c1ccc(-n2c3ccc(-c4nc(-c5ccc6ccccc6c5)nc(-c5cc6ccccc6c6ccccc56)n4)cc3c3c4cccc5c4n(c32)-c2ccccc2O5)cc1 ; c1ccc(-n2c3cccc(-c4nc(-c5ccc(-n6c7ccccc7c7ccccc76)cc5)nc(-c5cccc6ccccc56)n4)c3c3c4cccc5c4n(c32)-c2ccccc2O5)cc1]

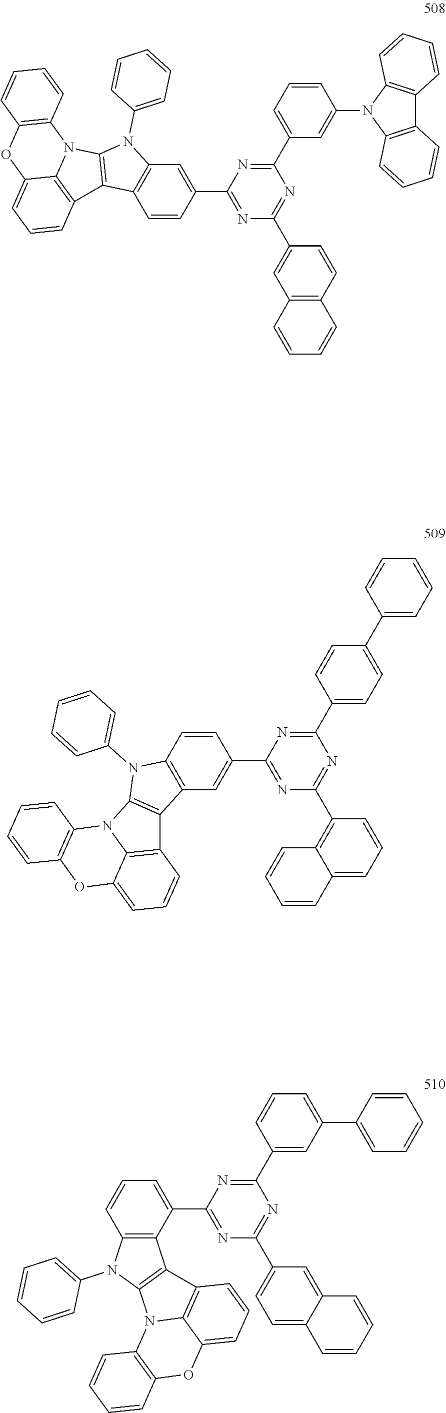 [SMILES: c1ccc(-n2c3cc(-c4nc(-c5cccc(-n6c7ccccc7c7ccccc76)c5)nc(-c5ccc6ccccc6c5)n4)ccc3c3c4cccc5c4n(c32)-c2ccccc2O5)cc1 ; c1ccc(-c2ccc(-c3nc(-c4ccc5c(c4)c4c6cccc7c6n(c4n5-c4ccccc4)-c4ccccc4O7)nc(-c4cccc5ccccc45)n3)cc2)cc1 ; c1ccc(-c2cccc(-c3nc(-c4ccc5ccccc5c4)nc(-c4cccc5c4c4c6cccc7c6n(c4n5-c4ccccc4)-c4ccccc4O7)n3)c2)cc1]

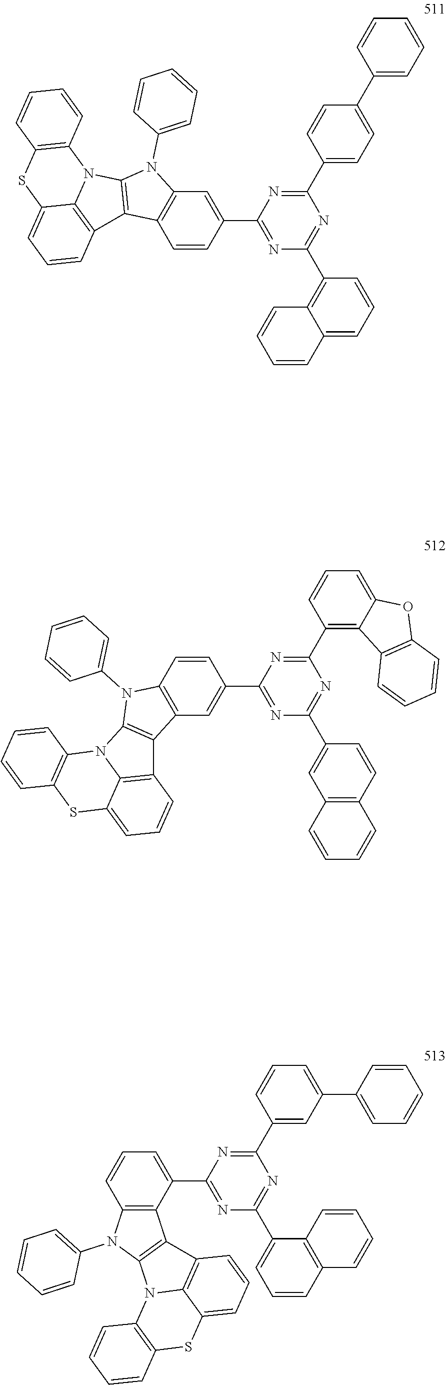 [SMILES: c1ccc(-c2ccc(-c3nc(-c4ccc5c6c7cccc8c7n(c6n(-c6ccccc6)c5c4)-c4ccccc4S8)nc(-c4cccc5ccccc45)n3)cc2)cc1 ; c1ccc(-n2c3ccc(-c4nc(-c5ccc6ccccc6c5)nc(-c5cccc6oc7ccccc7c56)n4)cc3c3c4cccc5c4n(c32)-c2ccccc2S5)cc1 ; c1ccc(-c2cccc(-c3nc(-c4cccc5ccccc45)nc(-c4cccc5c4c4c6cccc7c6n(c4n5-c4ccccc4)-c4ccccc4S7)n3)c2)cc1]

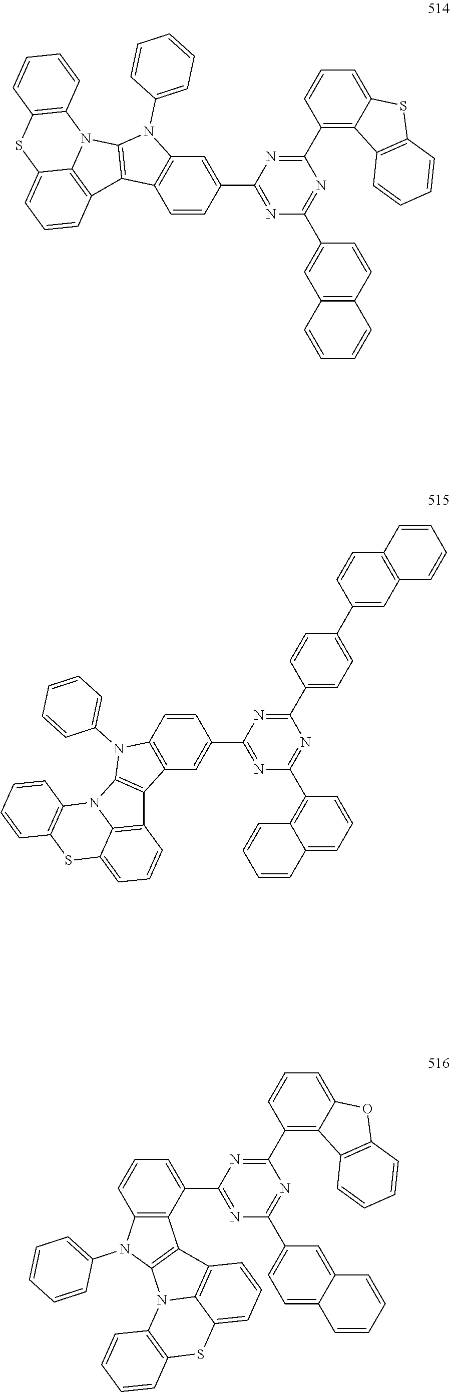 [SMILES: c1ccc(-n2c3cc(-c4nc(-c5ccc6ccccc6c5)nc(-c5cccc6sc7ccccc7c56)n4)ccc3c3c4cccc5c4n(c32)-c2ccccc2S5)cc1 ; c1ccc(-n2c3ccc(-c4nc(-c5ccc(-c6ccc7ccccc7c6)cc5)nc(-c5cccc6ccccc56)n4)cc3c3c4cccc5c4n(c32)-c2ccccc2S5)cc1 ; c1ccc(-n2c3cccc(-c4nc(-c5ccc6ccccc6c5)nc(-c5cccc6oc7ccccc7c56)n4)c3c3c4cccc5c4n(c32)-c2ccccc2S5)cc1]

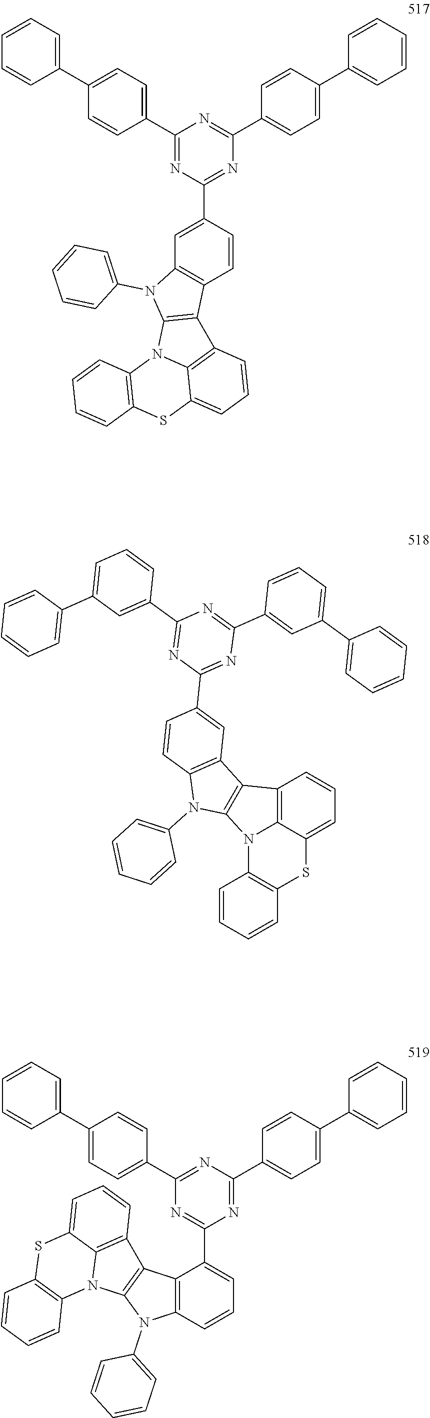 [SMILES: c1ccc(-c2ccc(-c3nc(-c4ccc(-c5ccccc5)cc4)nc(-c4ccc5c6c7cccc8c7n(c6n(-c6ccccc6)c5c4)-c4ccccc4S8)n3)cc2)cc1 ; c1ccc(-c2cccc(-c3nc(-c4cccc(-c5ccccc5)c4)nc(-c4ccc5c(c4)c4c6cccc7c6n(c4n5-c4ccccc4)-c4ccccc4S7)n3)c2)cc1 ; c1ccc(-c2ccc(-c3nc(-c4ccc(-c5ccccc5)cc4)nc(-c4cccc5c4c4c6cccc7c6n(c4n5-c4ccccc4)-c4ccccc4S7)n3)cc2)cc1]

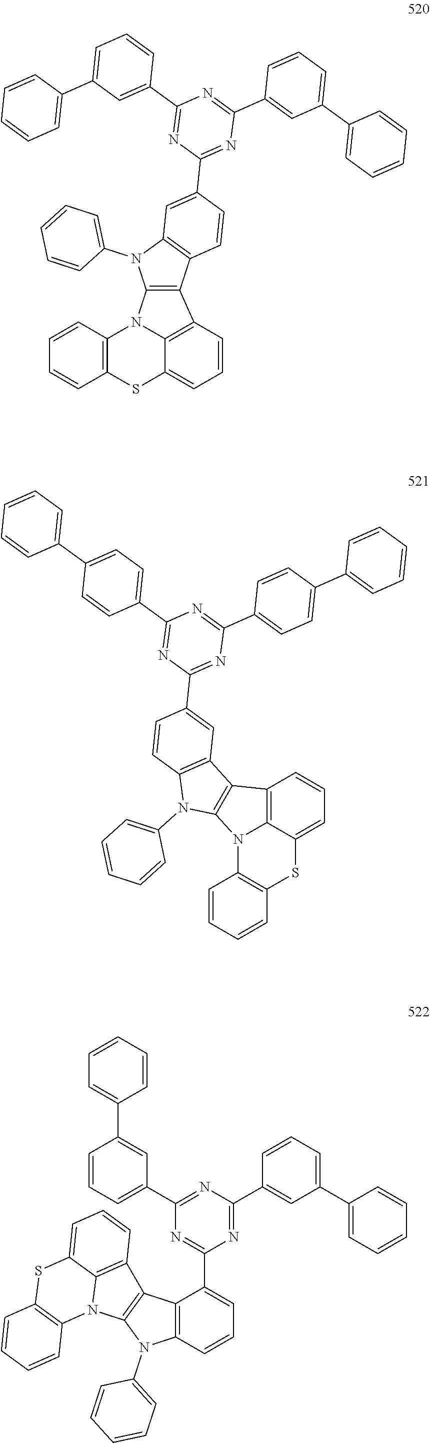 [SMILES: c1ccc(-c2cccc(-c3nc(-c4cccc(-c5ccccc5)c4)nc(-c4ccc5c6c7cccc8c7n(c6n(-c6ccccc6)c5c4)-c4ccccc4S8)n3)c2)cc1 ; c1ccc(-c2ccc(-c3nc(-c4ccc(-c5ccccc5)cc4)nc(-c4ccc5c(c4)c4c6cccc7c6n(c4n5-c4ccccc4)-c4ccccc4S7)n3)cc2)cc1 ; c1ccc(-c2cccc(-c3nc(-c4cccc(-c5ccccc5)c4)nc(-c4cccc5c4c4c6cccc7c6n(c4n5-c4ccccc4)-c4ccccc4S7)n3)c2)cc1]

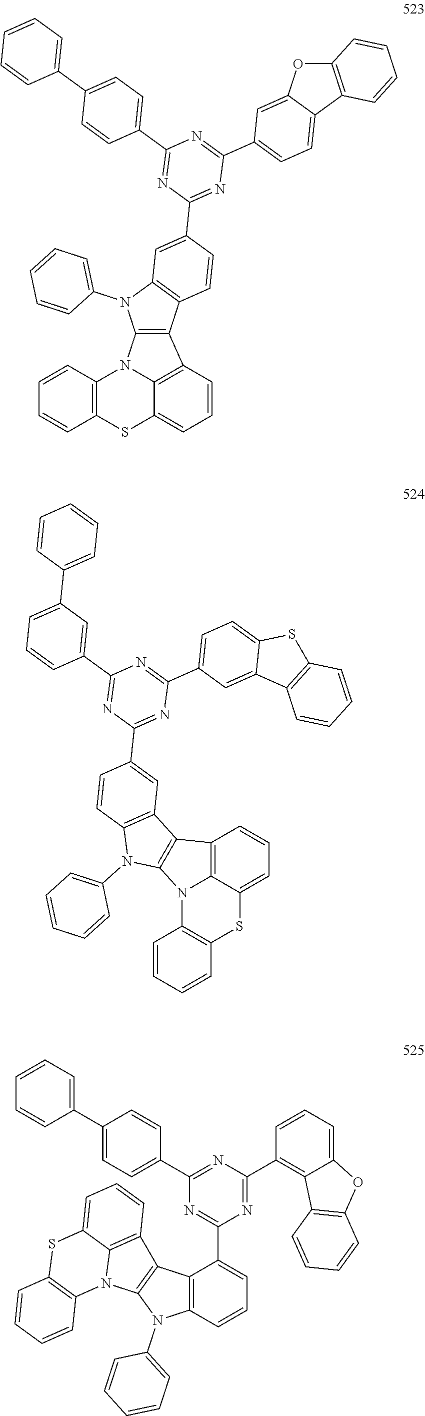 [SMILES: c1ccc(-c2ccc(-c3nc(-c4ccc5c(c4)oc4ccccc45)nc(-c4ccc5c6c7cccc8c7n(c6n(-c6ccccc6)c5c4)-c4ccccc4S8)n3)cc2)cc1 ; c1ccc(-c2cccc(-c3nc(-c4ccc5sc6ccccc6c5c4)nc(-c4ccc5c(c4)c4c6cccc7c6n(c4n5-c4ccccc4)-c4ccccc4S7)n3)c2)cc1 ; c1ccc(-c2ccc(-c3nc(-c4cccc5oc6ccccc6c45)nc(-c4cccc5c4c4c6cccc7c6n(c4n5-c4ccccc4)-c4ccccc4S7)n3)cc2)cc1]

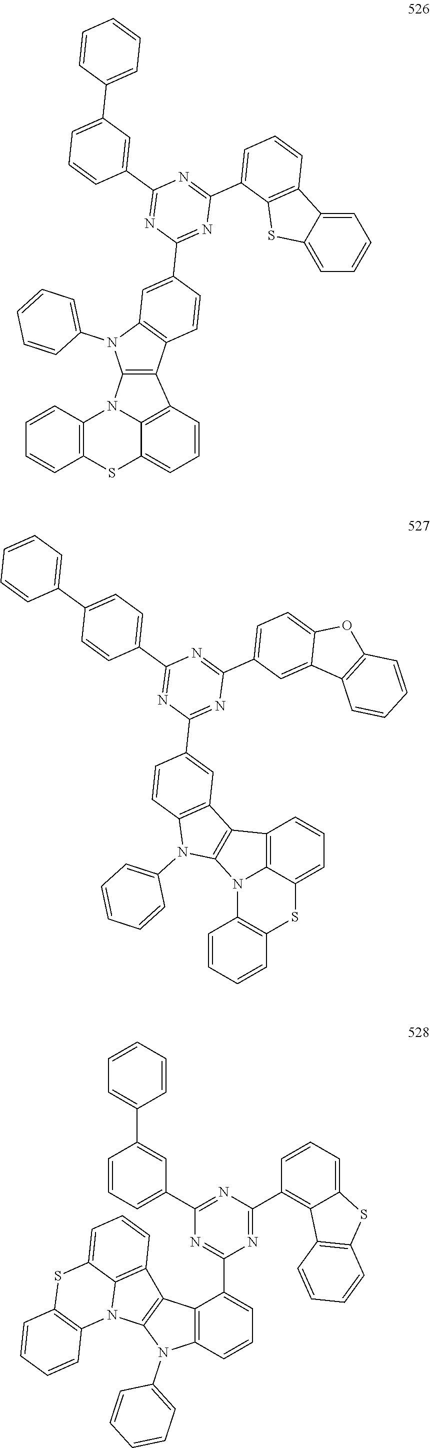 [SMILES: c1ccc(-c2cccc(-c3nc(-c4ccc5c6c7cccc8c7n(c6n(-c6ccccc6)c5c4)-c4ccccc4S8)nc(-c4cccc5c4sc4ccccc45)n3)c2)cc1 ; c1ccc(-c2ccc(-c3nc(-c4ccc5oc6ccccc6c5c4)nc(-c4ccc5c(c4)c4c6cccc7c6n(c4n5-c4ccccc4)-c4ccccc4S7)n3)cc2)cc1 ; c1ccc(-c2cccc(-c3nc(-c4cccc5sc6ccccc6c45)nc(-c4cccc5c4c4c6cccc7c6n(c4n5-c4ccccc4)-c4ccccc4S7)n3)c2)cc1]

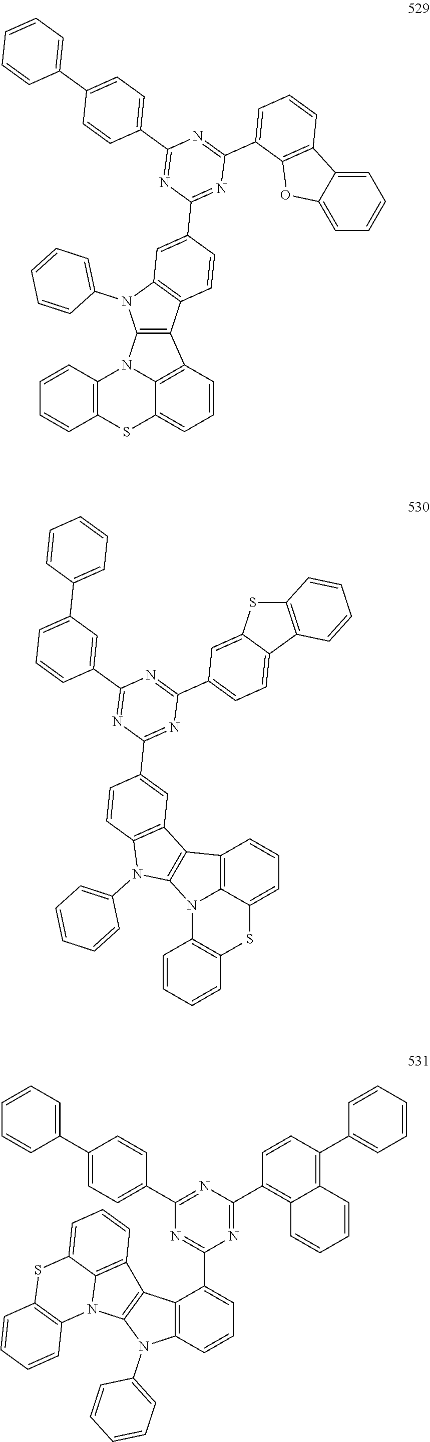 [SMILES: c1ccc(-c2ccc(-c3nc(-c4ccc5c6c7cccc8c7n(c6n(-c6ccccc6)c5c4)-c4ccccc4S8)nc(-c4cccc5c4oc4ccccc45)n3)cc2)cc1 ; c1ccc(-c2cccc(-c3nc(-c4ccc5c(c4)sc4ccccc45)nc(-c4ccc5c(c4)c4c6cccc7c6n(c4n5-c4ccccc4)-c4ccccc4S7)n3)c2)cc1 ; c1ccc(-c2ccc(-c3nc(-c4ccc(-c5ccccc5)c5ccccc45)nc(-c4cccc5c4c4c6cccc7c6n(c4n5-c4ccccc4)-c4ccccc4S7)n3)cc2)cc1]

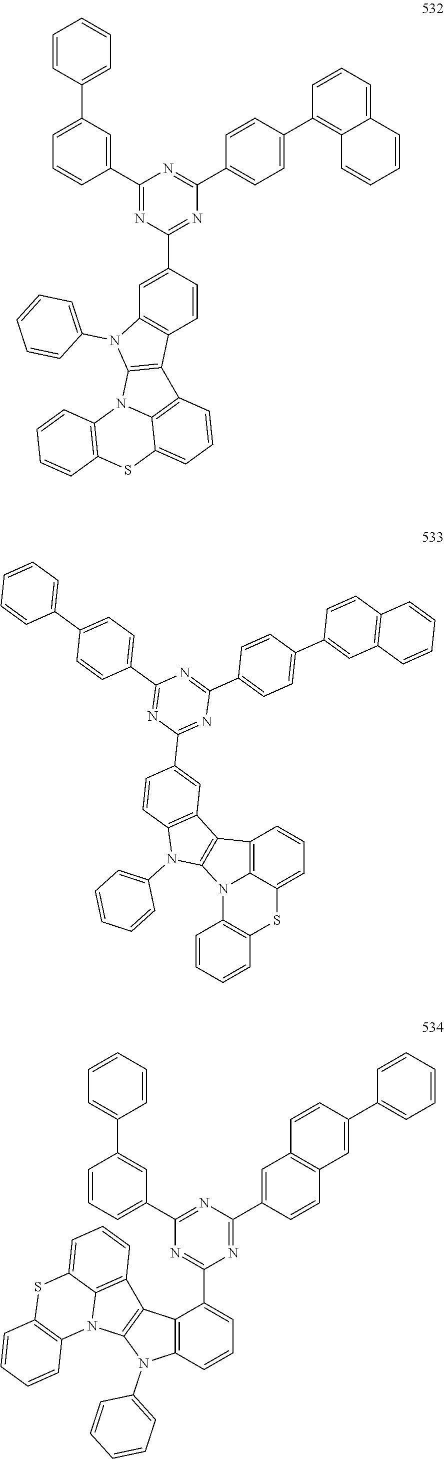 [SMILES: c1ccc(-c2cccc(-c3nc(-c4ccc(-c5cccc6ccccc56)cc4)nc(-c4ccc5c6c7cccc8c7n(c6n(-c6ccccc6)c5c4)-c4ccccc4S8)n3)c2)cc1 ; c1ccc(-c2ccc(-c3nc(-c4ccc(-c5ccc6ccccc6c5)cc4)nc(-c4ccc5c(c4)c4c6cccc7c6n(c4n5-c4ccccc4)-c4ccccc4S7)n3)cc2)cc1 ; c1ccc(-c2cccc(-c3nc(-c4ccc5cc(-c6ccccc6)ccc5c4)nc(-c4cccc5c4c4c6cccc7c6n(c4n5-c4ccccc4)-c4ccccc4S7)n3)c2)cc1]

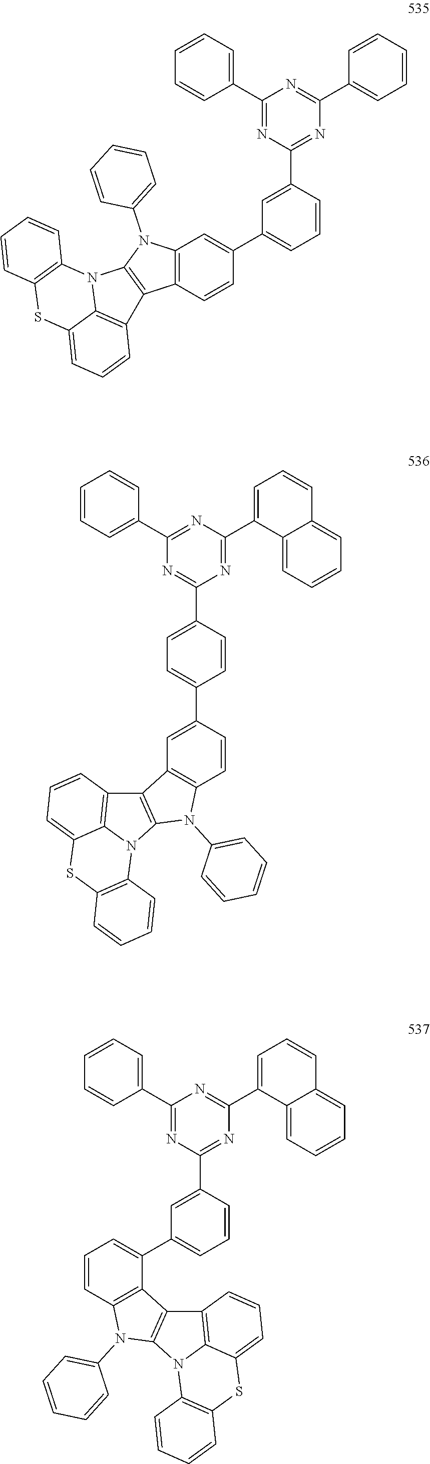 [SMILES: c1ccc(-c2nc(-c3ccccc3)nc(-c3cccc(-c4ccc5c6c7cccc8c7n(c6n(-c6ccccc6)c5c4)-c4ccccc4S8)c3)n2)cc1 ; c1ccc(-c2nc(-c3ccc(-c4ccc5c(c4)c4c6cccc7c6n(c4n5-c4ccccc4)-c4ccccc4S7)cc3)nc(-c3cccc4ccccc34)n2)cc1 ; c1ccc(-c2nc(-c3cccc(-c4cccc5c4c4c6cccc7c6n(c4n5-c4ccccc4)-c4ccccc4S7)c3)nc(-c3cccc4ccccc34)n2)cc1]

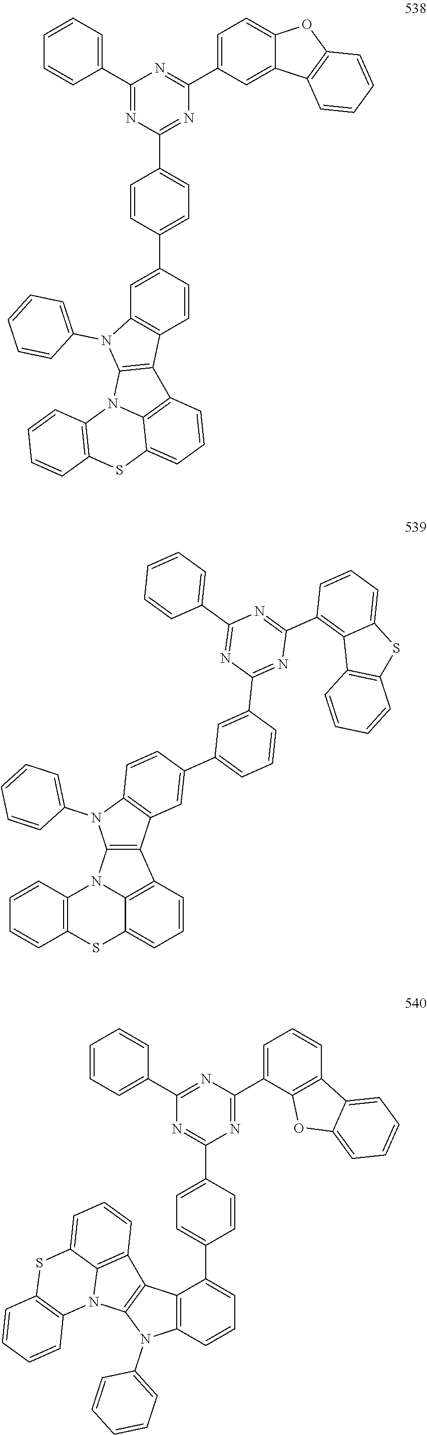 [SMILES: c1ccc(-c2nc(-c3ccc(-c4ccc5c6c7cccc8c7n(c6n(-c6ccccc6)c5c4)-c4ccccc4S8)cc3)nc(-c3ccc4oc5ccccc5c4c3)n2)cc1 ; c1ccc(-c2nc(-c3cccc(-c4ccc5c(c4)c4c6cccc7c6n(c4n5-c4ccccc4)-c4ccccc4S7)c3)nc(-c3cccc4sc5ccccc5c34)n2)cc1 ; c1ccc(-c2nc(-c3ccc(-c4cccc5c4c4c6cccc7c6n(c4n5-c4ccccc4)-c4ccccc4S7)cc3)nc(-c3cccc4c3oc3ccccc34)n2)cc1]

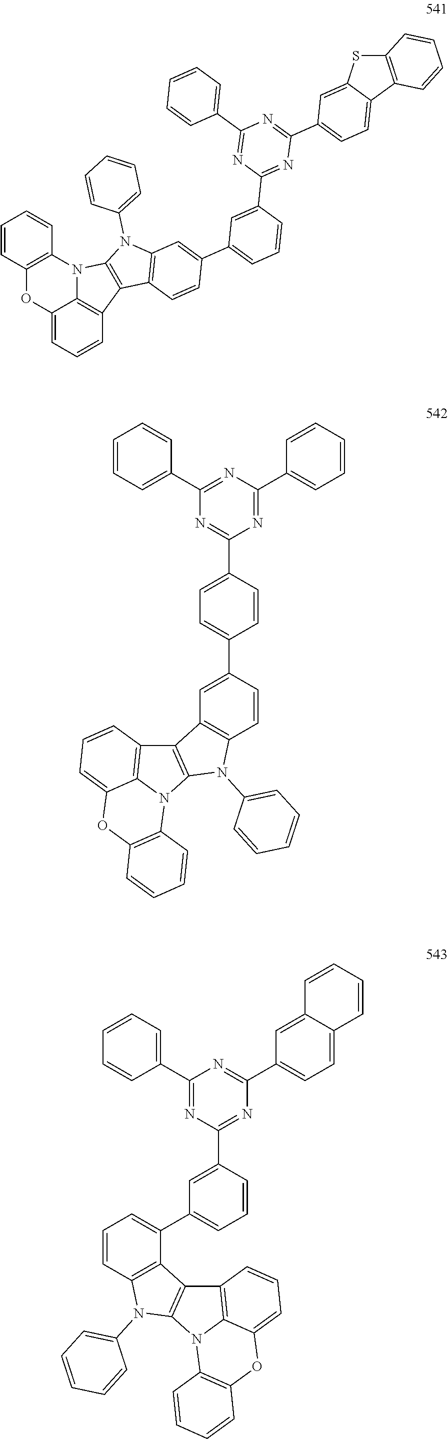 [SMILES: c1ccc(-c2nc(-c3cccc(-c4ccc5c6c7cccc8c7n(c6n(-c6ccccc6)c5c4)-c4ccccc4O8)c3)nc(-c3ccc4c(c3)sc3ccccc34)n2)cc1 ; c1ccc(-c2nc(-c3ccccc3)nc(-c3ccc(-c4ccc5c(c4)c4c6cccc7c6n(c4n5-c4ccccc4)-c4ccccc4O7)cc3)n2)cc1 ; c1ccc(-c2nc(-c3cccc(-c4cccc5c4c4c6cccc7c6n(c4n5-c4ccccc4)-c4ccccc4O7)c3)nc(-c3ccc4ccccc4c3)n2)cc1]

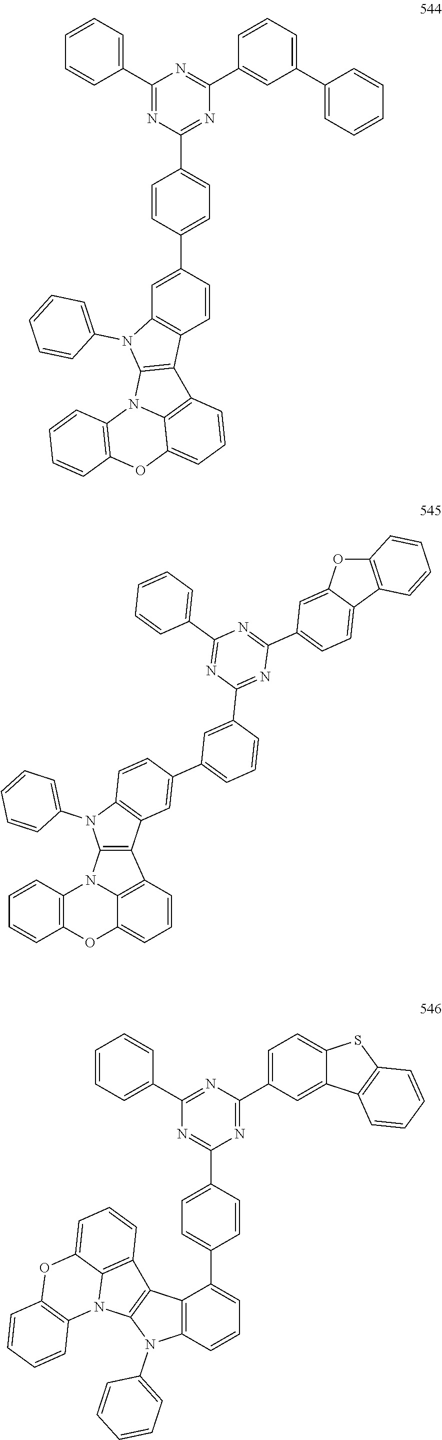 [SMILES: c1ccc(-c2cccc(-c3nc(-c4ccccc4)nc(-c4ccc(-c5ccc6c7c8cccc9c8n(c7n(-c7ccccc7)c6c5)-c5ccccc5O9)cc4)n3)c2)cc1 ; c1ccc(-c2nc(-c3cccc(-c4ccc5c(c4)c4c6cccc7c6n(c4n5-c4ccccc4)-c4ccccc4O7)c3)nc(-c3ccc4c(c3)oc3ccccc34)n2)cc1 ; c1ccc(-c2nc(-c3ccc(-c4cccc5c4c4c6cccc7c6n(c4n5-c4ccccc4)-c4ccccc4O7)cc3)nc(-c3ccc4sc5ccccc5c4c3)n2)cc1]

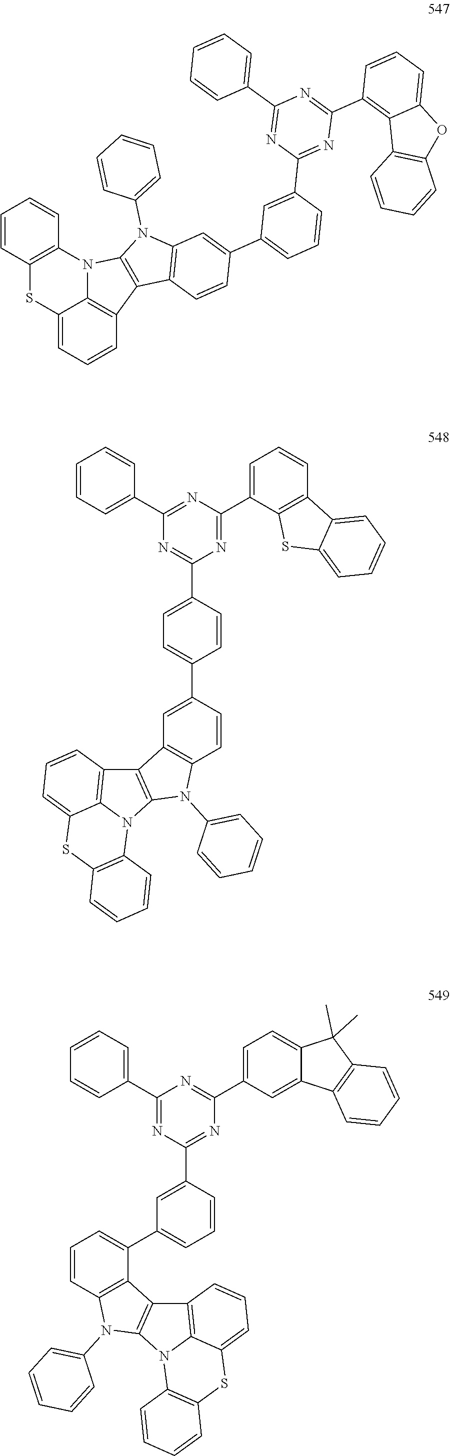 [SMILES: c1ccc(-c2nc(-c3cccc(-c4ccc5c6c7cccc8c7n(c6n(-c6ccccc6)c5c4)-c4ccccc4S8)c3)nc(-c3cccc4oc5ccccc5c34)n2)cc1 ; c1ccc(-c2nc(-c3ccc(-c4ccc5c(c4)c4c6cccc7c6n(c4n5-c4ccccc4)-c4ccccc4S7)cc3)nc(-c3cccc4c3sc3ccccc34)n2)cc1 ; CC1(C)c2ccccc2-c2cc(-c3nc(-c4ccccc4)nc(-c4cccc(-c5cccc6c5c5c7cccc8c7n(c5n6-c5ccccc5)-c5ccccc5S8)c4)n3)ccc21]

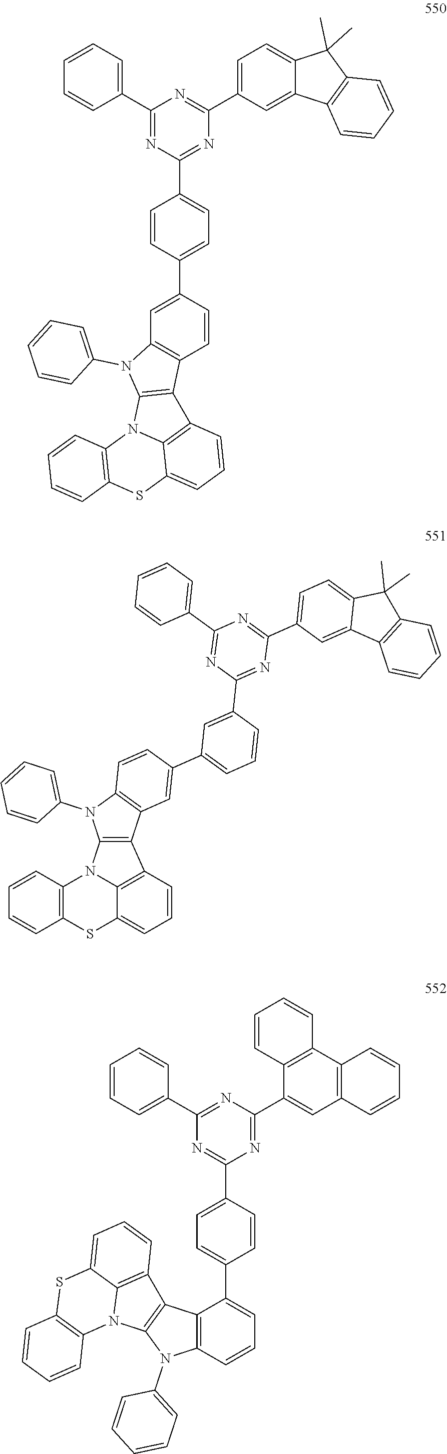 [SMILES: CC1(C)c2ccccc2-c2cc(-c3nc(-c4ccccc4)nc(-c4ccc(-c5ccc6c7c8cccc9c8n(c7n(-c7ccccc7)c6c5)-c5ccccc5S9)cc4)n3)ccc21 ; CC1(C)c2ccccc2-c2cc(-c3nc(-c4ccccc4)nc(-c4cccc(-c5ccc6c(c5)c5c7cccc8c7n(c5n6-c5ccccc5)-c5ccccc5S8)c4)n3)ccc21 ; c1ccc(-c2nc(-c3ccc(-c4cccc5c4c4c6cccc7c6n(c4n5-c4ccccc4)-c4ccccc4S7)cc3)nc(-c3cc4ccccc4c4ccccc34)n2)cc1]

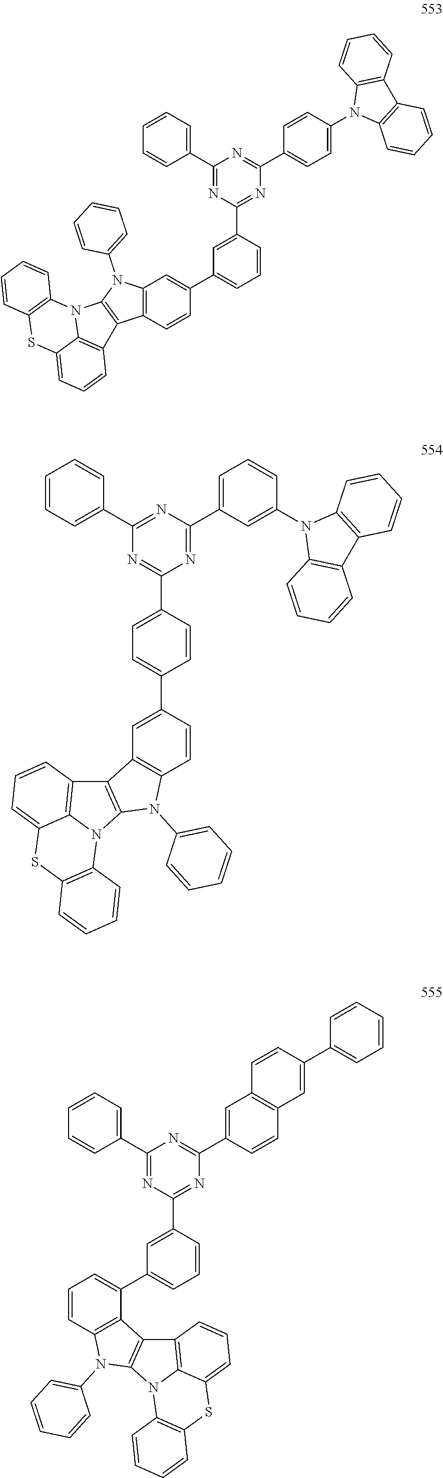 [SMILES: c1ccc(-c2nc(-c3ccc(-n4c5ccccc5c5ccccc54)cc3)nc(-c3cccc(-c4ccc5c6c7cccc8c7n(c6n(-c6ccccc6)c5c4)-c4ccccc4S8)c3)n2)cc1 ; c1ccc(-c2nc(-c3ccc(-c4ccc5c(c4)c4c6cccc7c6n(c4n5-c4ccccc4)-c4ccccc4S7)cc3)nc(-c3cccc(-n4c5ccccc5c5ccccc54)c3)n2)cc1 ; c1ccc(-c2ccc3cc(-c4nc(-c5ccccc5)nc(-c5cccc(-c6cccc7c6c6c8cccc9c8n(c6n7-c6ccccc6)-c6ccccc6S9)c5)n4)ccc3c2)cc1]

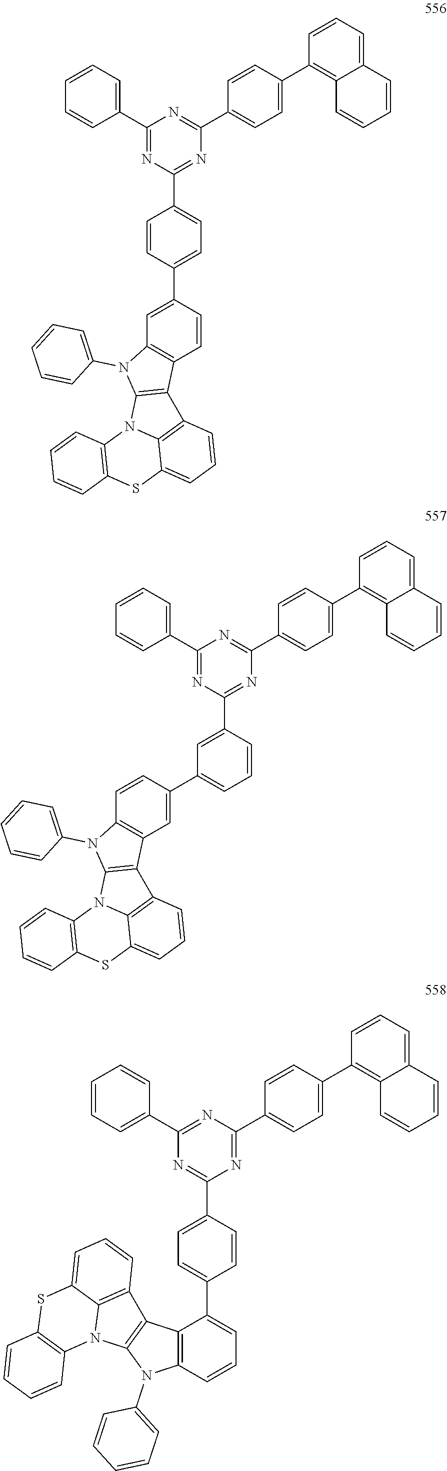 [SMILES: c1ccc(-c2nc(-c3ccc(-c4ccc5c6c7cccc8c7n(c6n(-c6ccccc6)c5c4)-c4ccccc4S8)cc3)nc(-c3ccc(-c4cccc5ccccc45)cc3)n2)cc1 ; c1ccc(-c2nc(-c3ccc(-c4cccc5ccccc45)cc3)nc(-c3cccc(-c4ccc5c(c4)c4c6cccc7c6n(c4n5-c4ccccc4)-c4ccccc4S7)c3)n2)cc1 ; c1ccc(-c2nc(-c3ccc(-c4cccc5ccccc45)cc3)nc(-c3ccc(-c4cccc5c4c4c6cccc7c6n(c4n5-c4ccccc4)-c4ccccc4S7)cc3)n2)cc1]

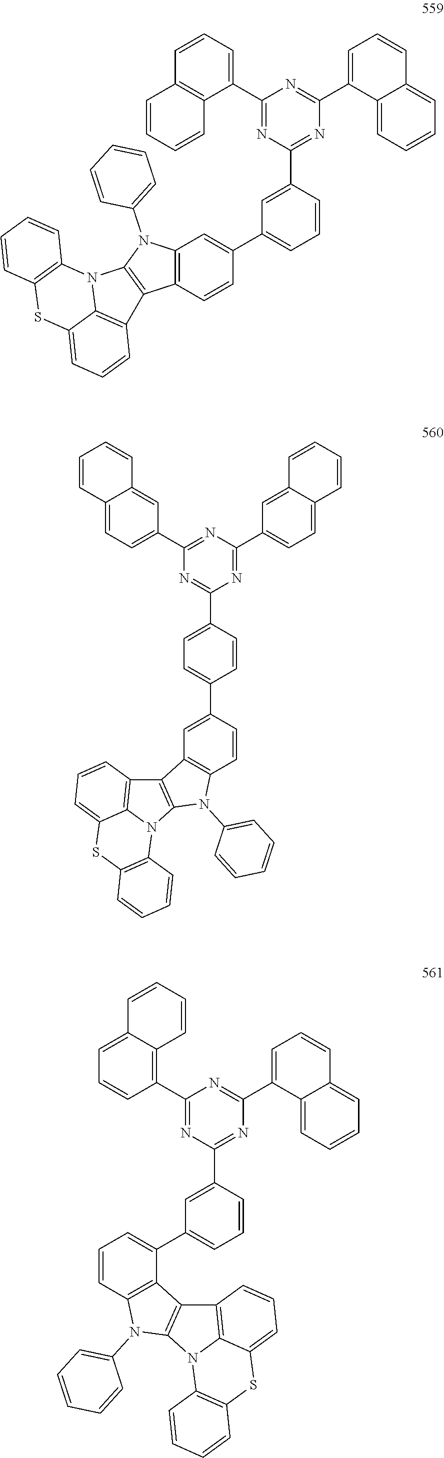 [SMILES: c1ccc(-n2c3cc(-c4cccc(-c5nc(-c6cccc7ccccc67)nc(-c6cccc7ccccc67)n5)c4)ccc3c3c4cccc5c4n(c32)-c2ccccc2S5)cc1 ; c1ccc(-n2c3ccc(-c4ccc(-c5nc(-c6ccc7ccccc7c6)nc(-c6ccc7ccccc7c6)n5)cc4)cc3c3c4cccc5c4n(c32)-c2ccccc2S5)cc1 ; c1ccc(-n2c3cccc(-c4cccc(-c5nc(-c6cccc7ccccc67)nc(-c6cccc7ccccc67)n5)c4)c3c3c4cccc5c4n(c32)-c2ccccc2S5)cc1]

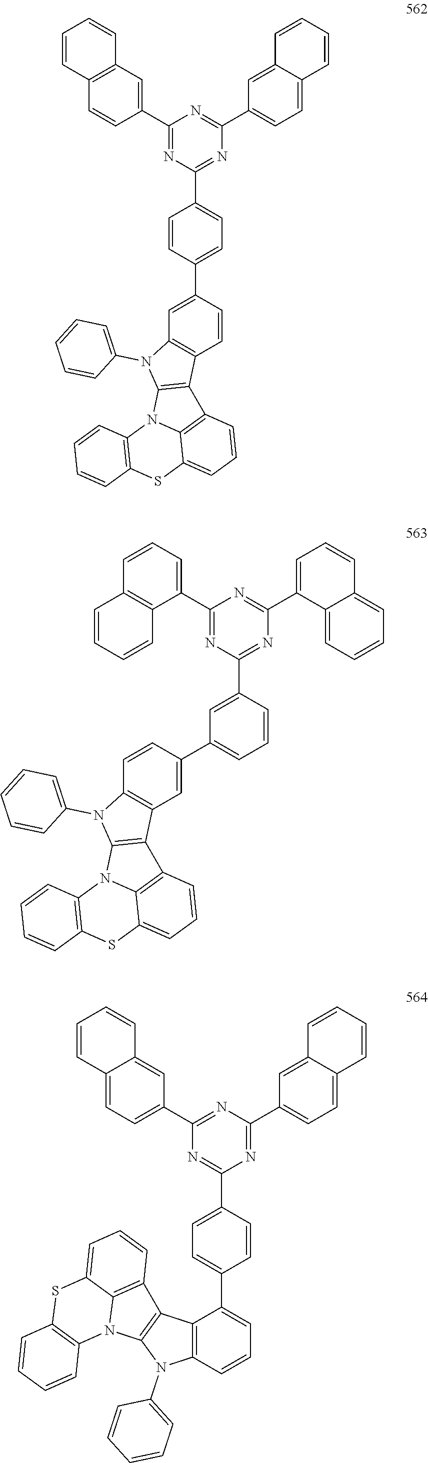 [SMILES: c1ccc(-n2c3cc(-c4ccc(-c5nc(-c6ccc7ccccc7c6)nc(-c6ccc7ccccc7c6)n5)cc4)ccc3c3c4cccc5c4n(c32)-c2ccccc2S5)cc1 ; c1ccc(-n2c3ccc(-c4cccc(-c5nc(-c6cccc7ccccc67)nc(-c6cccc7ccccc67)n5)c4)cc3c3c4cccc5c4n(c32)-c2ccccc2S5)cc1 ; c1ccc(-n2c3cccc(-c4ccc(-c5nc(-c6ccc7ccccc7c6)nc(-c6ccc7ccccc7c6)n5)cc4)c3c3c4cccc5c4n(c32)-c2ccccc2S5)cc1]

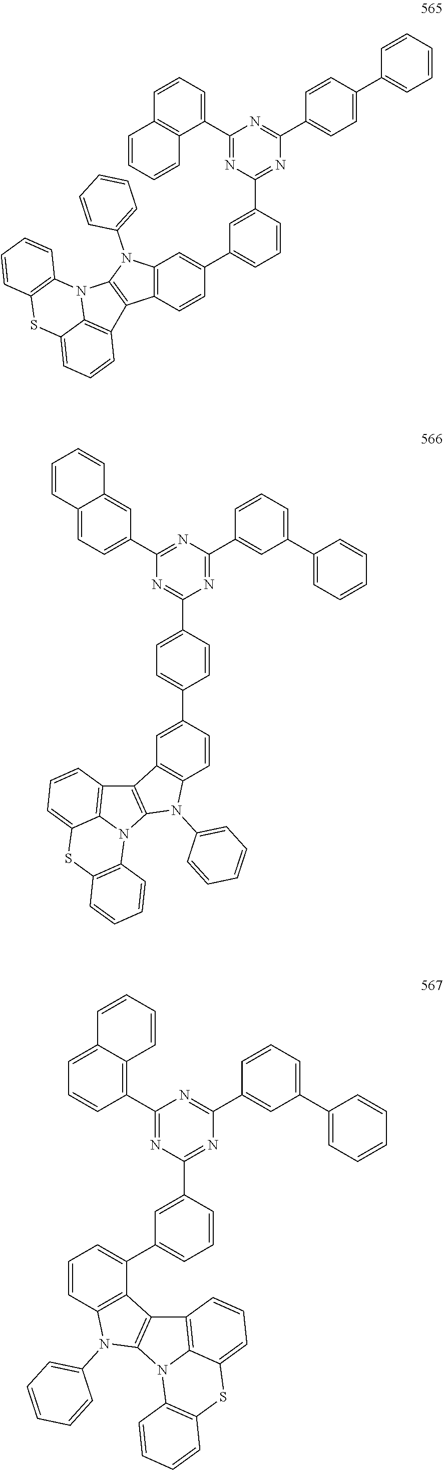 [SMILES: c1ccc(-c2ccc(-c3nc(-c4cccc(-c5ccc6c7c8cccc9c8n(c7n(-c7ccccc7)c6c5)-c5ccccc5S9)c4)nc(-c4cccc5ccccc45)n3)cc2)cc1 ; c1ccc(-c2cccc(-c3nc(-c4ccc(-c5ccc6c(c5)c5c7cccc8c7n(c5n6-c5ccccc5)-c5ccccc5S8)cc4)nc(-c4ccc5ccccc5c4)n3)c2)cc1 ; c1ccc(-c2cccc(-c3nc(-c4cccc(-c5cccc6c5c5c7cccc8c7n(c5n6-c5ccccc5)-c5ccccc5S8)c4)nc(-c4cccc5ccccc45)n3)c2)cc1]

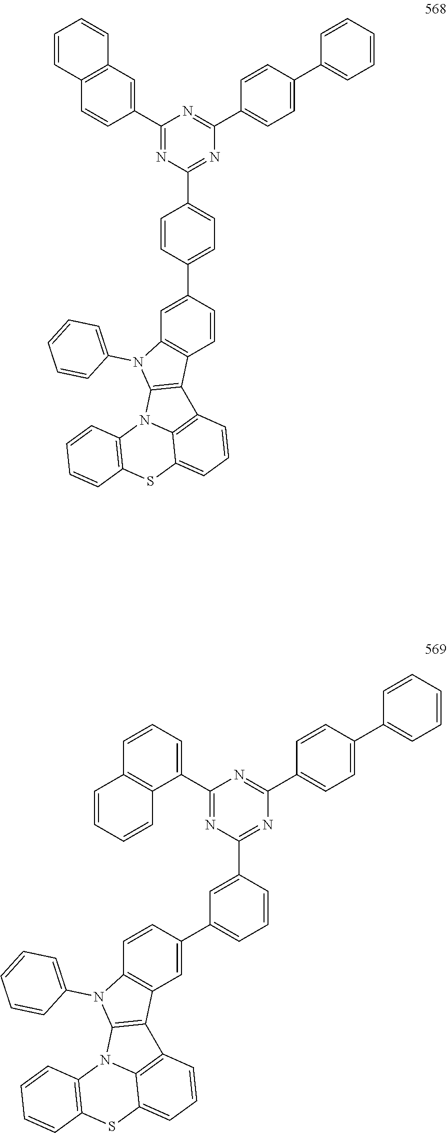 [SMILES: c1ccc(-c2ccc(-c3nc(-c4ccc(-c5ccc6c7c8cccc9c8n(c7n(-c7ccccc7)c6c5)-c5ccccc5S9)cc4)nc(-c4ccc5ccccc5c4)n3)cc2)cc1 ; c1ccc(-c2ccc(-c3nc(-c4cccc(-c5ccc6c(c5)c5c7cccc8c7n(c5n6-c5ccccc5)-c5ccccc5S8)c4)nc(-c4cccc5ccccc45)n3)cc2)cc1]

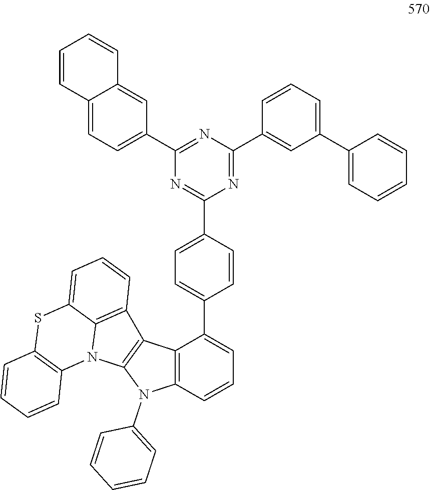 [SMILES: c1ccc(-c2cccc(-c3nc(-c4ccc(-c5cccc6c5c5c7cccc8c7n(c5n6-c5ccccc5)-c5ccccc5S8)cc4)nc(-c4ccc5ccccc5c4)n3)c2)cc1]

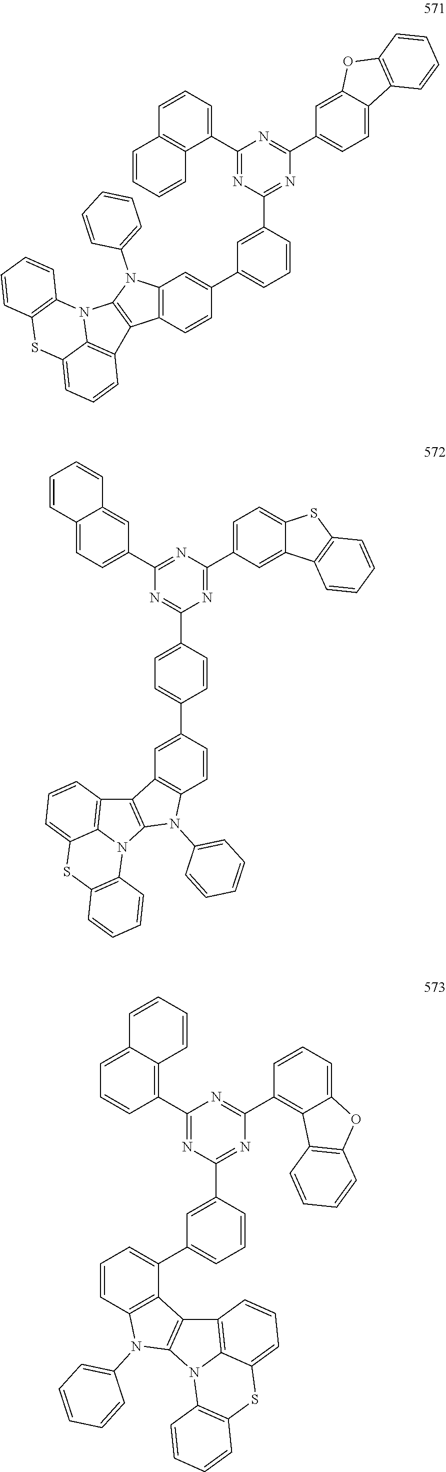 [SMILES: c1ccc(-n2c3cc(-c4cccc(-c5nc(-c6ccc7c(c6)oc6ccccc67)nc(-c6cccc7ccccc67)n5)c4)ccc3c3c4cccc5c4n(c32)-c2ccccc2S5)cc1 ; c1ccc(-n2c3ccc(-c4ccc(-c5nc(-c6ccc7ccccc7c6)nc(-c6ccc7sc8ccccc8c7c6)n5)cc4)cc3c3c4cccc5c4n(c32)-c2ccccc2S5)cc1 ; c1ccc(-n2c3cccc(-c4cccc(-c5nc(-c6cccc7ccccc67)nc(-c6cccc7oc8ccccc8c67)n5)c4)c3c3c4cccc5c4n(c32)-c2ccccc2S5)cc1]

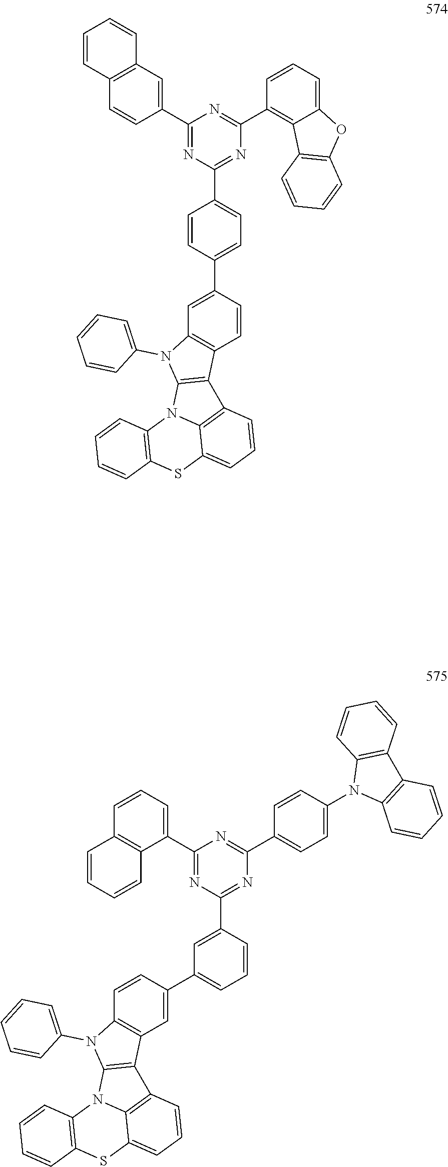 [SMILES: c1ccc(-n2c3cc(-c4ccc(-c5nc(-c6ccc7ccccc7c6)nc(-c6cccc7oc8ccccc8c67)n5)cc4)ccc3c3c4cccc5c4n(c32)-c2ccccc2S5)cc1 ; c1ccc(-n2c3ccc(-c4cccc(-c5nc(-c6ccc(-n7c8ccccc8c8ccccc87)cc6)nc(-c6cccc7ccccc67)n5)c4)cc3c3c4cccc5c4n(c32)-c2ccccc2S5)cc1]

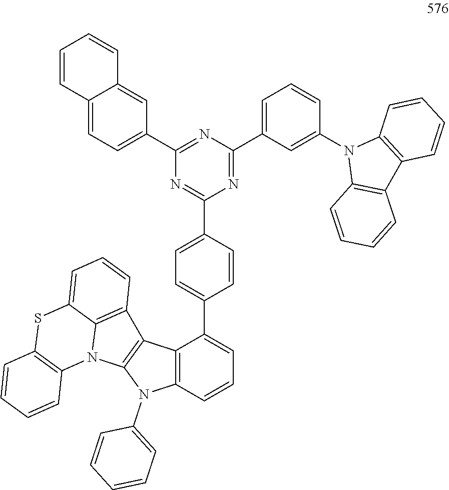 [SMILES: c1ccc(-n2c3cccc(-c4ccc(-c5nc(-c6cccc(-n7c8ccccc8c8ccccc87)c6)nc(-c6ccc7ccccc7c6)n5)cc4)c3c3c4cccc5c4n(c32)-c2ccccc2S5)cc1]

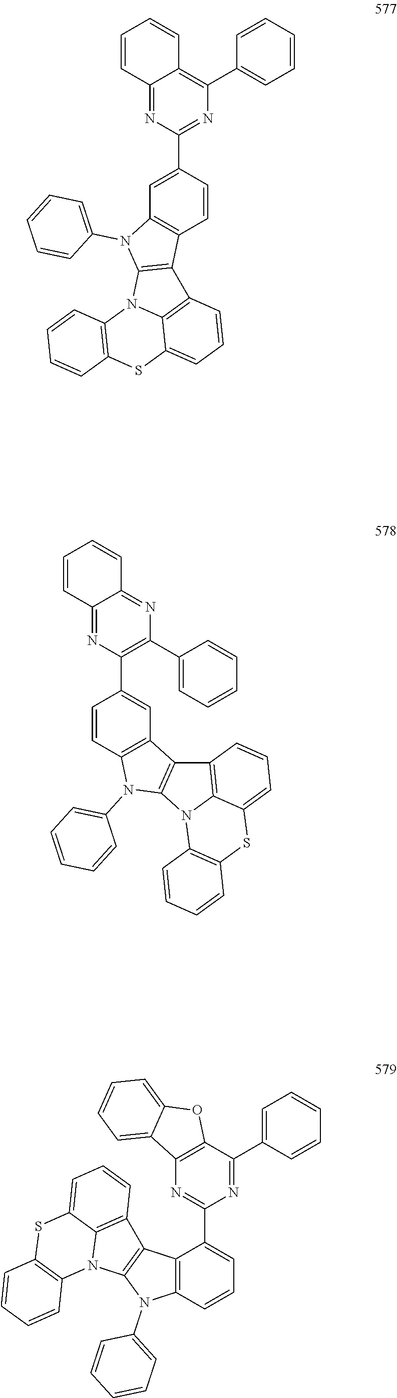 [SMILES: c1ccc(-c2nc(-c3ccc4c5c6cccc7c6n(c5n(-c5ccccc5)c4c3)-c3ccccc3S7)nc3ccccc23)cc1 ; c1ccc(-c2nc3ccccc3nc2-c2ccc3c(c2)c2c4cccc5c4n(c2n3-c2ccccc2)-c2ccccc2S5)cc1 ; c1ccc(-c2nc(-c3cccc4c3c3c5cccc6c5n(c3n4-c3ccccc3)-c3ccccc3S6)nc3c2oc2ccccc23)cc1]

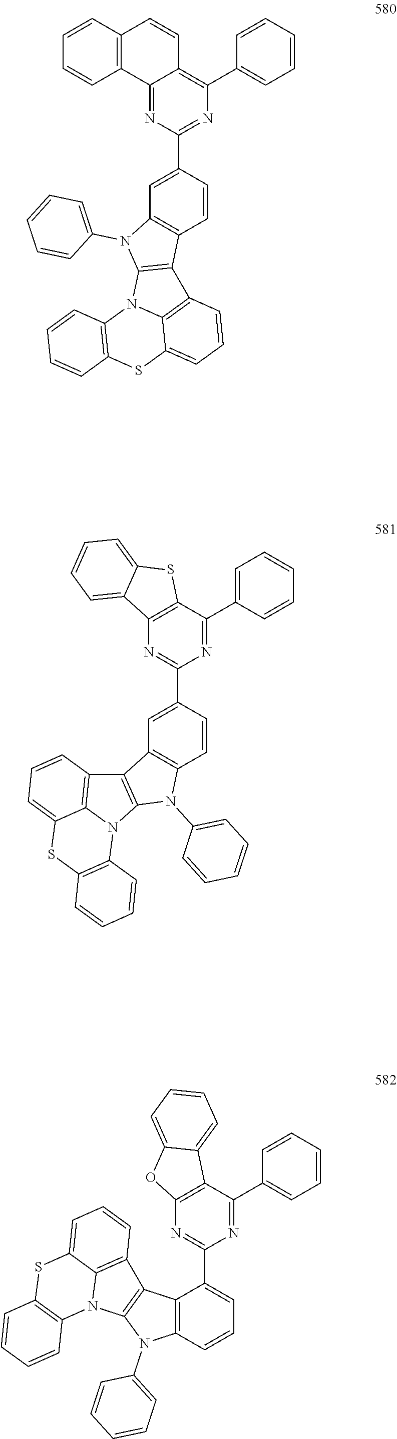 [SMILES: c1ccc(-c2nc(-c3ccc4c5c6cccc7c6n(c5n(-c5ccccc5)c4c3)-c3ccccc3S7)nc3c2ccc2ccccc23)cc1 ; c1ccc(-c2nc(-c3ccc4c(c3)c3c5cccc6c5n(c3n4-c3ccccc3)-c3ccccc3S6)nc3c2sc2ccccc23)cc1 ; c1ccc(-c2nc(-c3cccc4c3c3c5cccc6c5n(c3n4-c3ccccc3)-c3ccccc3S6)nc3oc4ccccc4c23)cc1]

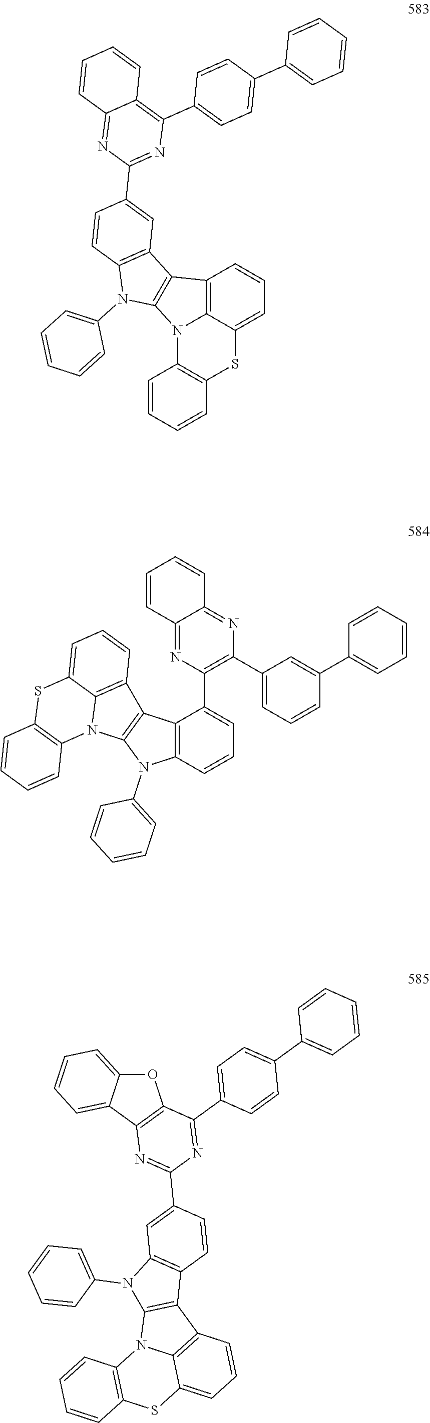 [SMILES: c1ccc(-c2ccc(-c3nc(-c4ccc5c(c4)c4c6cccc7c6n(c4n5-c4ccccc4)-c4ccccc4S7)nc4ccccc34)cc2)cc1 ; c1ccc(-c2cccc(-c3nc4ccccc4nc3-c3cccc4c3c3c5cccc6c5n(c3n4-c3ccccc3)-c3ccccc3S6)c2)cc1 ; c1ccc(-c2ccc(-c3nc(-c4ccc5c6c7cccc8c7n(c6n(-c6ccccc6)c5c4)-c4ccccc4S8)nc4c3oc3ccccc34)cc2)cc1]

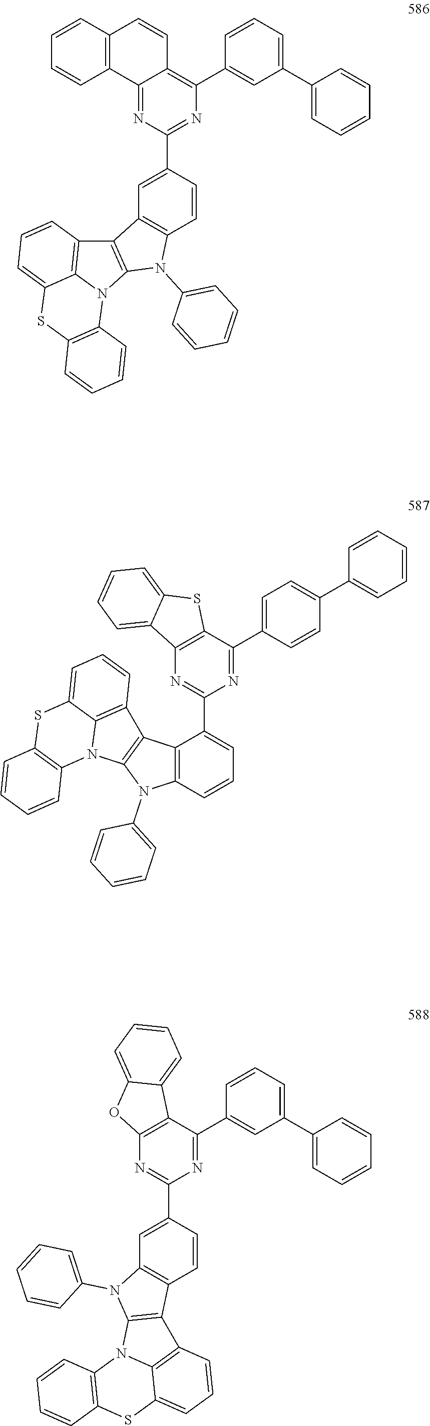 [SMILES: c1ccc(-c2cccc(-c3nc(-c4ccc5c(c4)c4c6cccc7c6n(c4n5-c4ccccc4)-c4ccccc4S7)nc4c3ccc3ccccc34)c2)cc1 ; c1ccc(-c2ccc(-c3nc(-c4cccc5c4c4c6cccc7c6n(c4n5-c4ccccc4)-c4ccccc4S7)nc4c3sc3ccccc34)cc2)cc1 ; c1ccc(-c2cccc(-c3nc(-c4ccc5c6c7cccc8c7n(c6n(-c6ccccc6)c5c4)-c4ccccc4S8)nc4oc5ccccc5c34)c2)cc1]

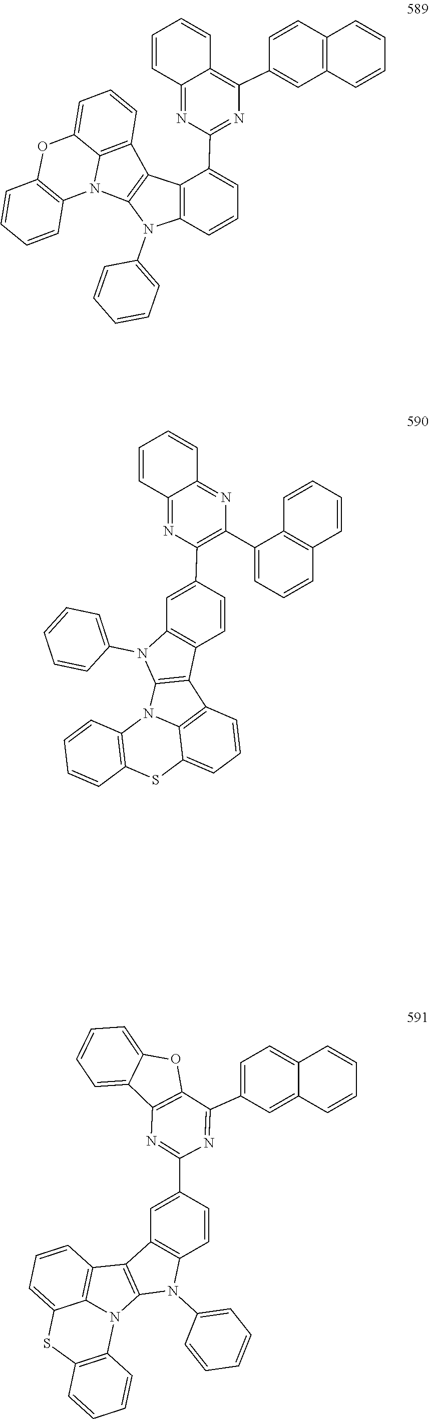 [SMILES: c1ccc(-n2c3cccc(-c4nc(-c5ccc6ccccc6c5)c5ccccc5n4)c3c3c4cccc5c4n(c32)-c2ccccc2O5)cc1 ; c1ccc(-n2c3cc(-c4nc5ccccc5nc4-c4cccc5ccccc45)ccc3c3c4cccc5c4n(c32)-c2ccccc2S5)cc1 ; c1ccc(-n2c3ccc(-c4nc(-c5ccc6ccccc6c5)c5oc6ccccc6c5n4)cc3c3c4cccc5c4n(c32)-c2ccccc2S5)cc1]

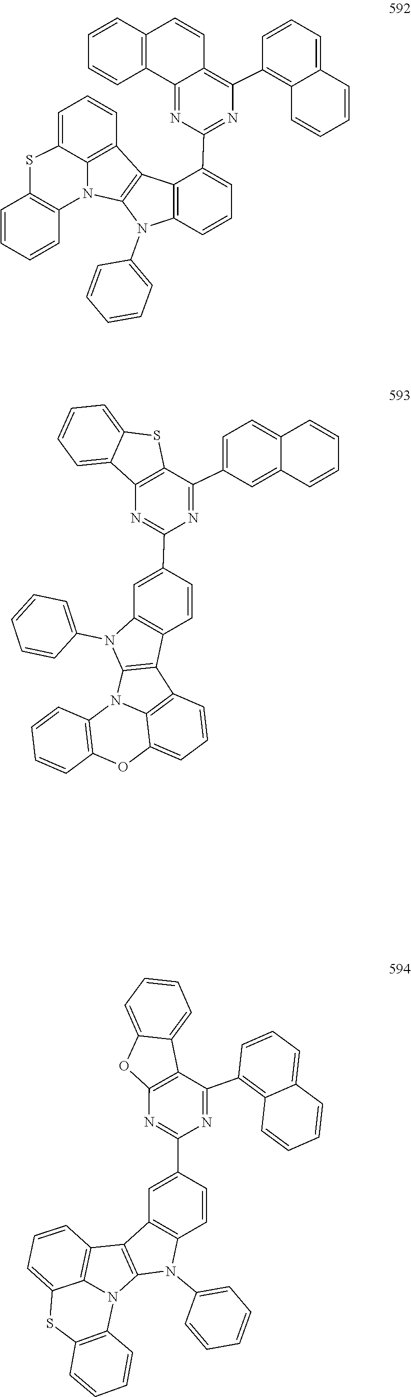 [SMILES: c1ccc(-n2c3cccc(-c4nc(-c5cccc6ccccc56)c5ccc6ccccc6c5n4)c3c3c4cccc5c4n(c32)-c2ccccc2S5)cc1 ; c1ccc(-n2c3cc(-c4nc(-c5ccc6ccccc6c5)c5sc6ccccc6c5n4)ccc3c3c4cccc5c4n(c32)-c2ccccc2O5)cc1 ; c1ccc(-n2c3ccc(-c4nc(-c5cccc6ccccc56)c5c(n4)oc4ccccc45)cc3c3c4cccc5c4n(c32)-c2ccccc2S5)cc1]

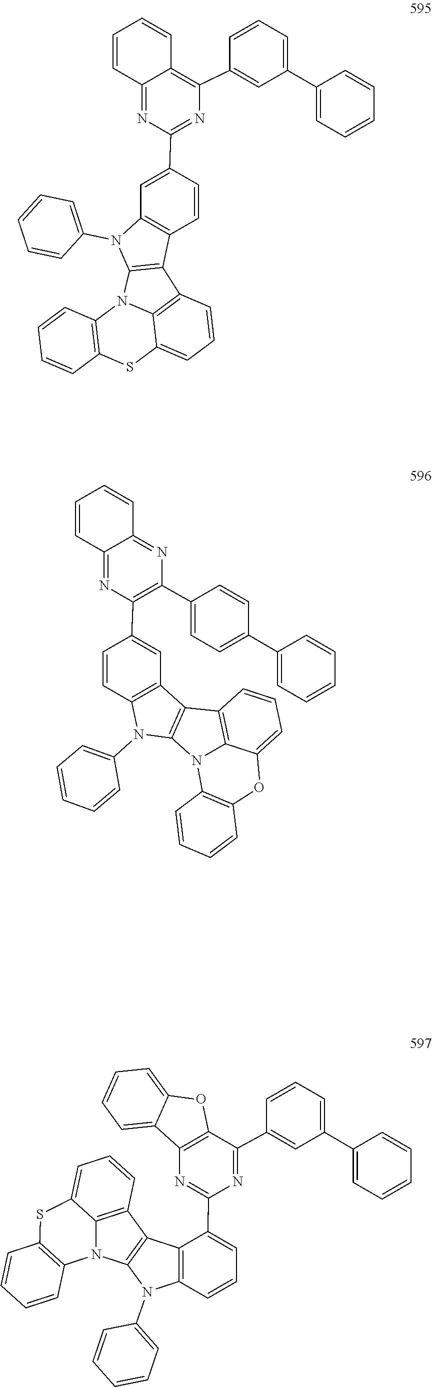 [SMILES: c1ccc(-c2cccc(-c3nc(-c4ccc5c6c7cccc8c7n(c6n(-c6ccccc6)c5c4)-c4ccccc4S8)nc4ccccc34)c2)cc1 ; c1ccc(-c2ccc(-c3nc4ccccc4nc3-c3ccc4c(c3)c3c5cccc6c5n(c3n4-c3ccccc3)-c3ccccc3O6)cc2)cc1 ; c1ccc(-c2cccc(-c3nc(-c4cccc5c4c4c6cccc7c6n(c4n5-c4ccccc4)-c4ccccc4S7)nc4c3oc3ccccc34)c2)cc1]

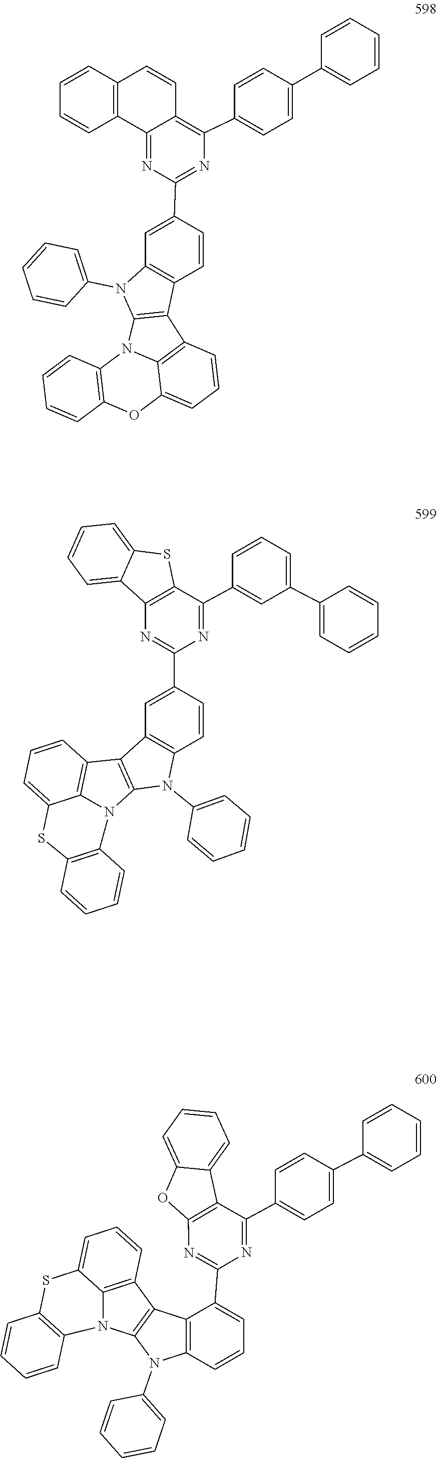 [SMILES: c1ccc(-c2ccc(-c3nc(-c4ccc5c6c7cccc8c7n(c6n(-c6ccccc6)c5c4)-c4ccccc4O8)nc4c3ccc3ccccc34)cc2)cc1 ; c1ccc(-c2cccc(-c3nc(-c4ccc5c(c4)c4c6cccc7c6n(c4n5-c4ccccc4)-c4ccccc4S7)nc4c3sc3ccccc34)c2)cc1 ; c1ccc(-c2ccc(-c3nc(-c4cccc5c4c4c6cccc7c6n(c4n5-c4ccccc4)-c4ccccc4S7)nc4oc5ccccc5c34)cc2)cc1]

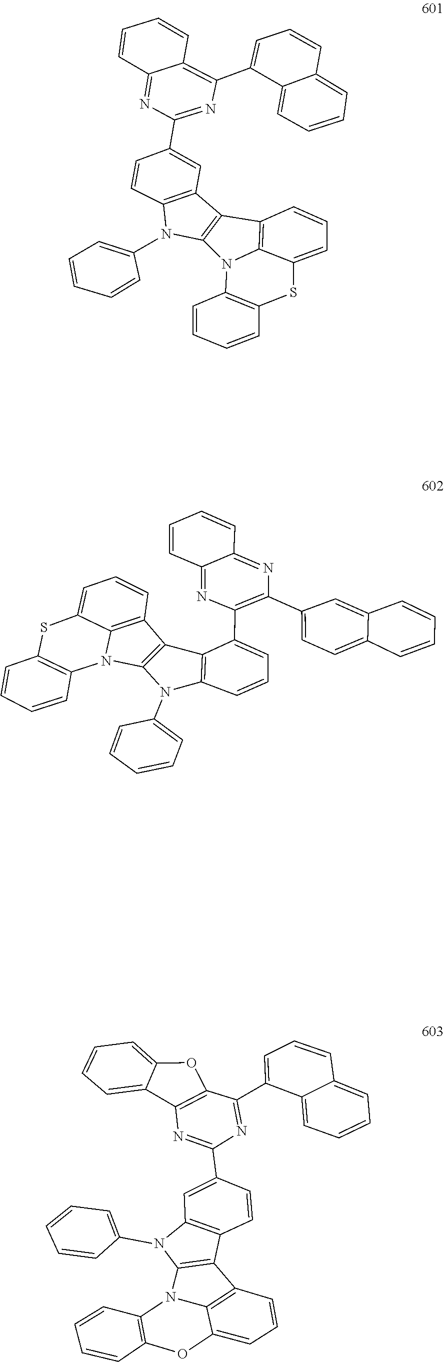 [SMILES: c1ccc(-n2c3ccc(-c4nc(-c5cccc6ccccc56)c5ccccc5n4)cc3c3c4cccc5c4n(c32)-c2ccccc2S5)cc1 ; c1ccc(-n2c3cccc(-c4nc5ccccc5nc4-c4ccc5ccccc5c4)c3c3c4cccc5c4n(c32)-c2ccccc2S5)cc1 ; c1ccc(-n2c3cc(-c4nc(-c5cccc6ccccc56)c5oc6ccccc6c5n4)ccc3c3c4cccc5c4n(c32)-c2ccccc2O5)cc1]

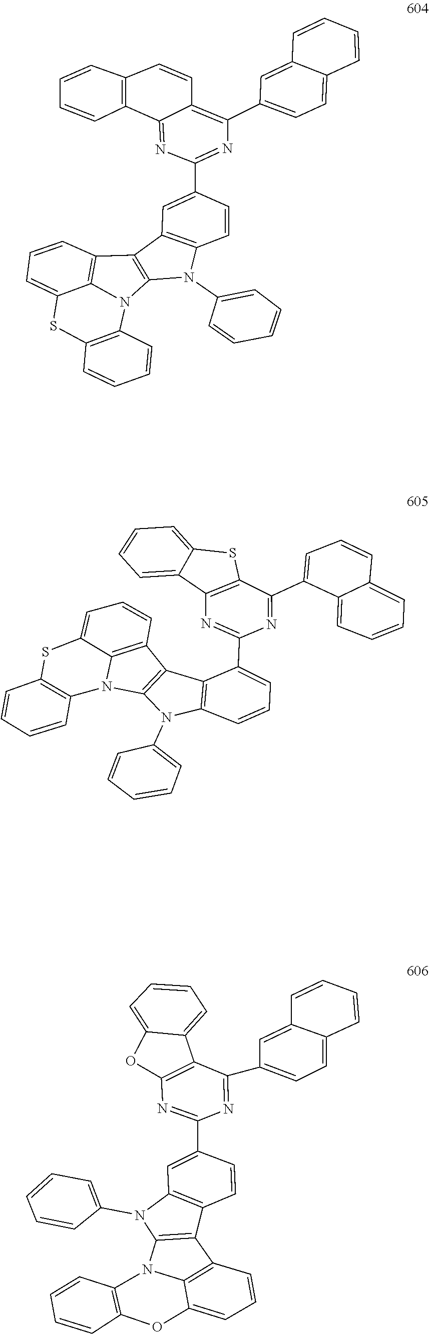 [SMILES: c1ccc(-n2c3ccc(-c4nc(-c5ccc6ccccc6c5)c5ccc6ccccc6c5n4)cc3c3c4cccc5c4n(c32)-c2ccccc2S5)cc1 ; c1ccc(-n2c3cccc(-c4nc(-c5cccc6ccccc56)c5sc6ccccc6c5n4)c3c3c4cccc5c4n(c32)-c2ccccc2S5)cc1 ; c1ccc(-n2c3cc(-c4nc(-c5ccc6ccccc6c5)c5c(n4)oc4ccccc45)ccc3c3c4cccc5c4n(c32)-c2ccccc2O5)cc1]

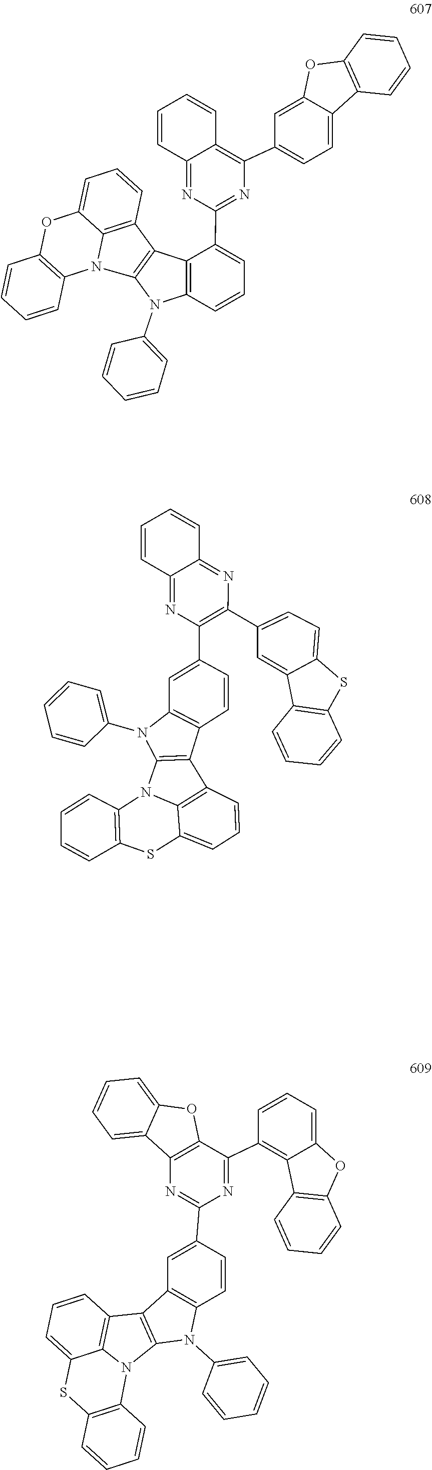 [SMILES: c1ccc(-n2c3cccc(-c4nc(-c5ccc6c(c5)oc5ccccc56)c5ccccc5n4)c3c3c4cccc5c4n(c32)-c2ccccc2O5)cc1 ; c1ccc(-n2c3cc(-c4nc5ccccc5nc4-c4ccc5sc6ccccc6c5c4)ccc3c3c4cccc5c4n(c32)-c2ccccc2S5)cc1 ; c1ccc(-n2c3ccc(-c4nc(-c5cccc6oc7ccccc7c56)c5oc6ccccc6c5n4)cc3c3c4cccc5c4n(c32)-c2ccccc2S5)cc1]

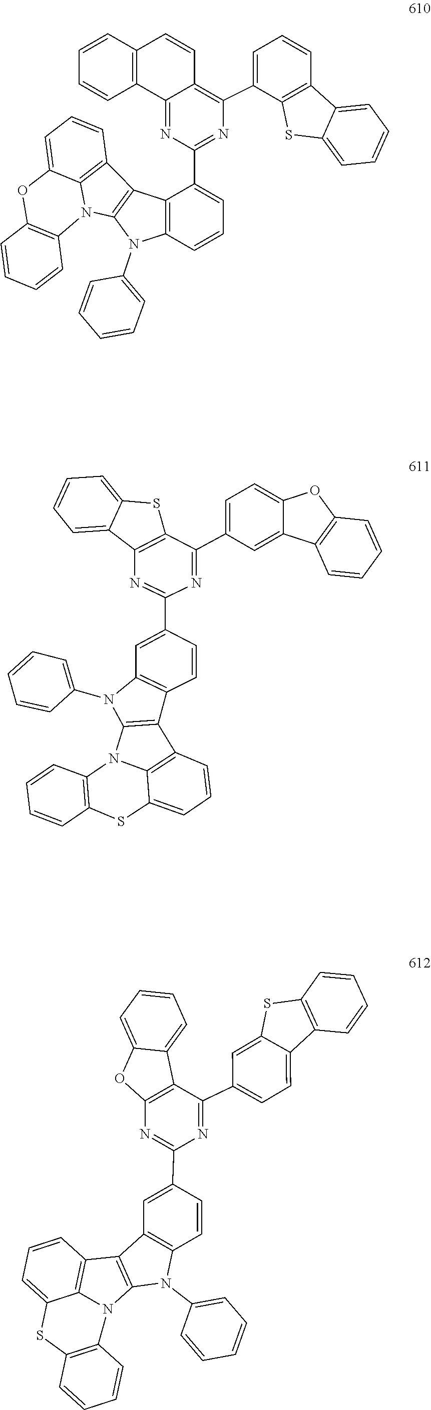 [SMILES: c1ccc(-n2c3cccc(-c4nc(-c5cccc6c5sc5ccccc56)c5ccc6ccccc6c5n4)c3c3c4cccc5c4n(c32)-c2ccccc2O5)cc1 ; c1ccc(-n2c3cc(-c4nc(-c5ccc6oc7ccccc7c6c5)c5sc6ccccc6c5n4)ccc3c3c4cccc5c4n(c32)-c2ccccc2S5)cc1 ; c1ccc(-n2c3ccc(-c4nc(-c5ccc6c(c5)sc5ccccc56)c5c(n4)oc4ccccc45)cc3c3c4cccc5c4n(c32)-c2ccccc2S5)cc1]

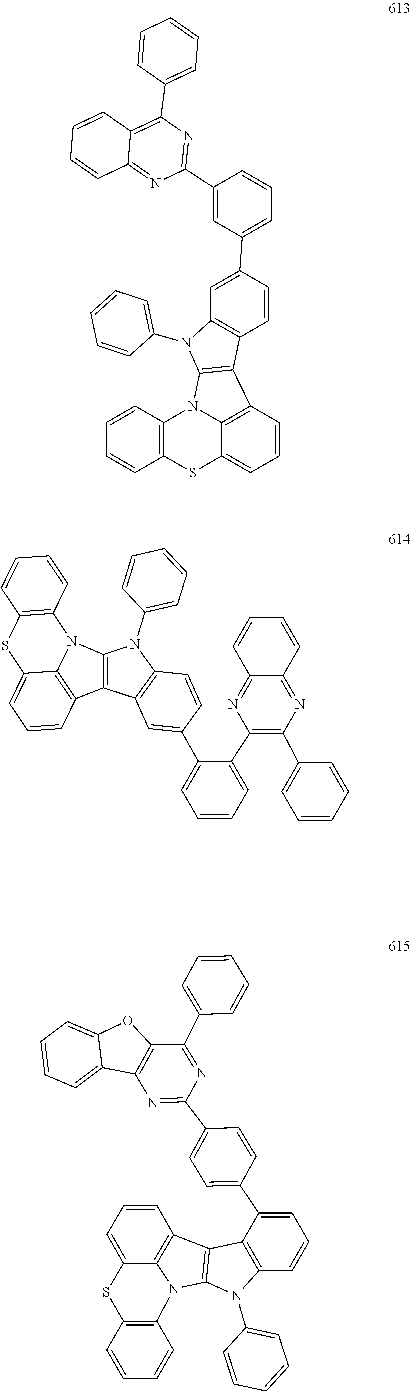 [SMILES: c1ccc(-c2nc(-c3cccc(-c4ccc5c6c7cccc8c7n(c6n(-c6ccccc6)c5c4)-c4ccccc4S8)c3)nc3ccccc23)cc1 ; c1ccc(-c2nc3ccccc3nc2-c2ccccc2-c2ccc3c(c2)c2c4cccc5c4n(c2n3-c2ccccc2)-c2ccccc2S5)cc1 ; c1ccc(-c2nc(-c3ccc(-c4cccc5c4c4c6cccc7c6n(c4n5-c4ccccc4)-c4ccccc4S7)cc3)nc3c2oc2ccccc23)cc1]

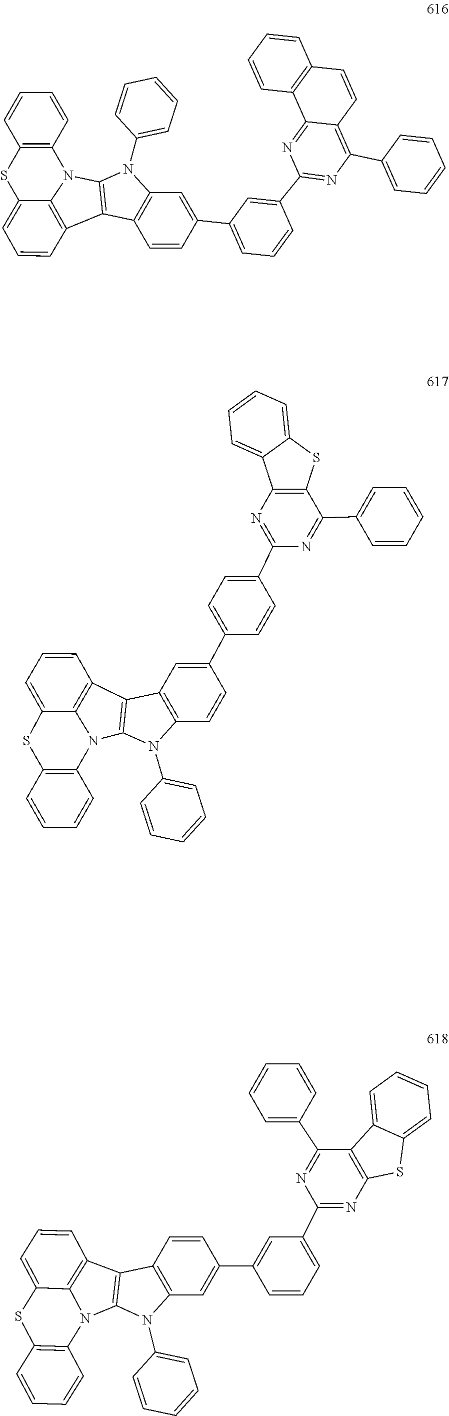 [SMILES: c1ccc(-c2nc(-c3cccc(-c4ccc5c6c7cccc8c7n(c6n(-c6ccccc6)c5c4)-c4ccccc4S8)c3)nc3c2ccc2ccccc23)cc1 ; c1ccc(-c2nc(-c3ccc(-c4ccc5c(c4)c4c6cccc7c6n(c4n5-c4ccccc4)-c4ccccc4S7)cc3)nc3c2sc2ccccc23)cc1 ; c1ccc(-c2nc(-c3cccc(-c4ccc5c6c7cccc8c7n(c6n(-c6ccccc6)c5c4)-c4ccccc4S8)c3)nc3sc4ccccc4c23)cc1]

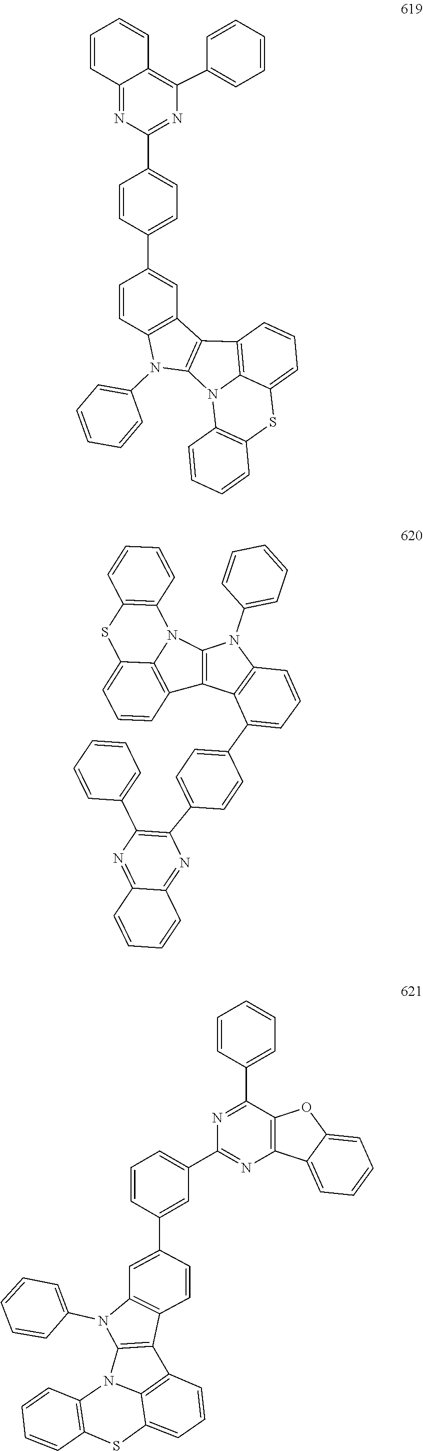 [SMILES: c1ccc(-c2nc(-c3ccc(-c4ccc5c(c4)c4c6cccc7c6n(c4n5-c4ccccc4)-c4ccccc4S7)cc3)nc3ccccc23)cc1 ; c1ccc(-c2nc3ccccc3nc2-c2ccc(-c3cccc4c3c3c5cccc6c5n(c3n4-c3ccccc3)-c3ccccc3S6)cc2)cc1 ; c1ccc(-c2nc(-c3cccc(-c4ccc5c6c7cccc8c7n(c6n(-c6ccccc6)c5c4)-c4ccccc4S8)c3)nc3c2oc2ccccc23)cc1]

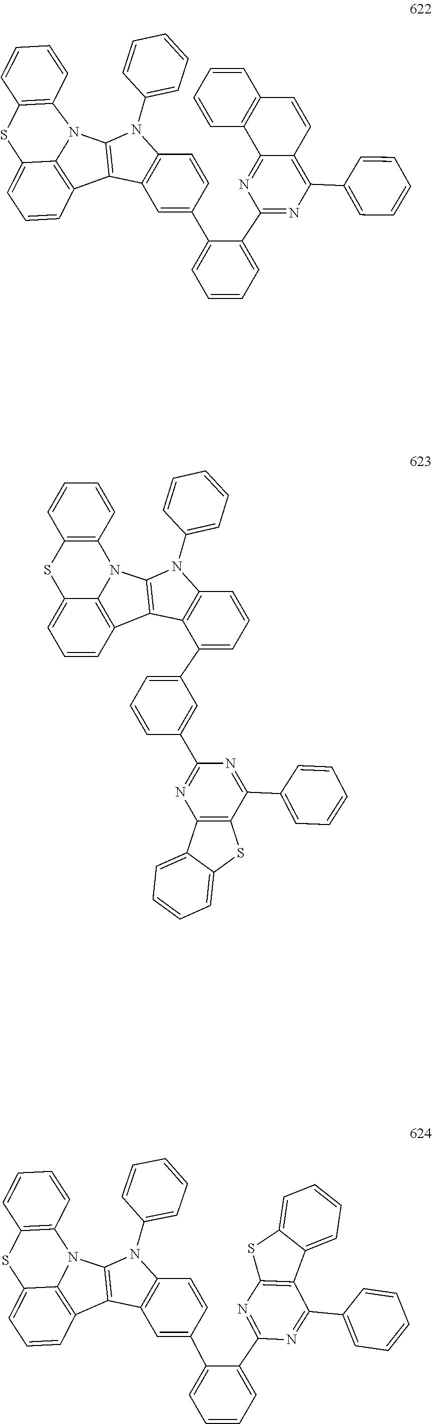 [SMILES: c1ccc(-c2nc(-c3ccccc3-c3ccc4c(c3)c3c5cccc6c5n(c3n4-c3ccccc3)-c3ccccc3S6)nc3c2ccc2ccccc23)cc1 ; c1ccc(-c2nc(-c3cccc(-c4cccc5c4c4c6cccc7c6n(c4n5-c4ccccc4)-c4ccccc4S7)c3)nc3c2sc2ccccc23)cc1 ; c1ccc(-c2nc(-c3ccccc3-c3ccc4c(c3)c3c5cccc6c5n(c3n4-c3ccccc3)-c3ccccc3S6)nc3sc4ccccc4c23)cc1]

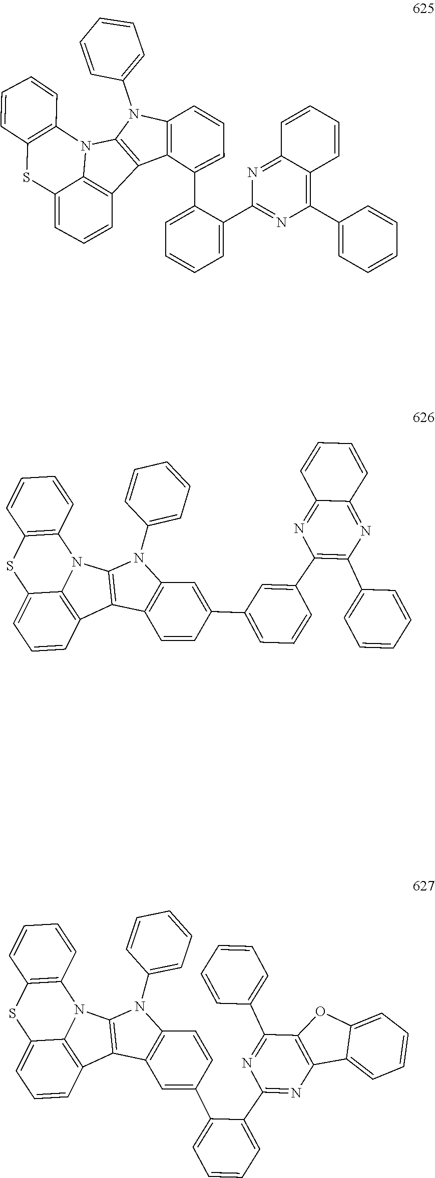 [SMILES: c1ccc(-c2nc(-c3ccccc3-c3cccc4c3c3c5cccc6c5n(c3n4-c3ccccc3)-c3ccccc3S6)nc3ccccc23)cc1 ; c1ccc(-c2nc3ccccc3nc2-c2cccc(-c3ccc4c5c6cccc7c6n(c5n(-c5ccccc5)c4c3)-c3ccccc3S7)c2)cc1 ; c1ccc(-c2nc(-c3ccccc3-c3ccc4c(c3)c3c5cccc6c5n(c3n4-c3ccccc3)-c3ccccc3S6)nc3c2oc2ccccc23)cc1]

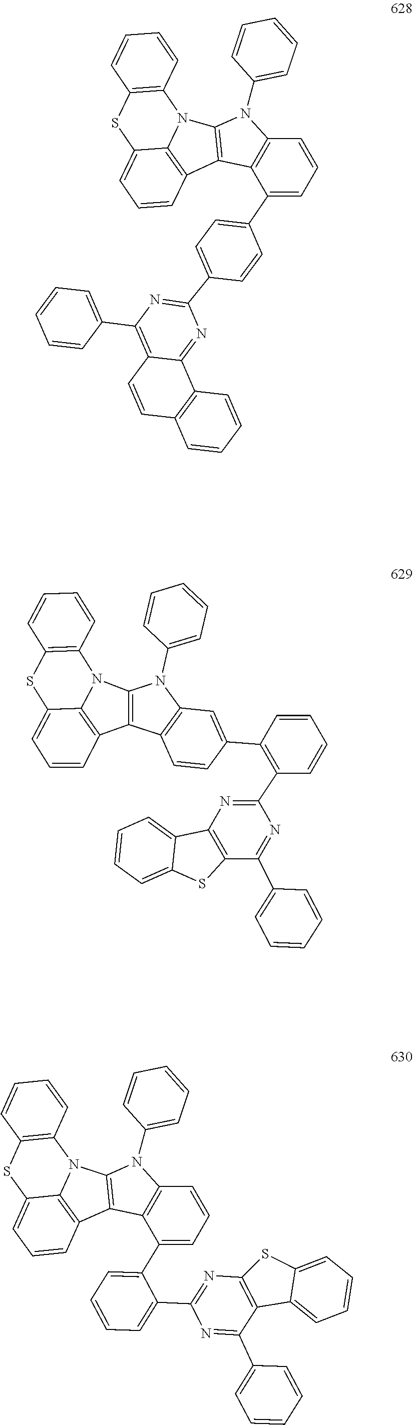 [SMILES: c1ccc(-c2nc(-c3ccc(-c4cccc5c4c4c6cccc7c6n(c4n5-c4ccccc4)-c4ccccc4S7)cc3)nc3c2ccc2ccccc23)cc1 ; c1ccc(-c2nc(-c3ccccc3-c3ccc4c5c6cccc7c6n(c5n(-c5ccccc5)c4c3)-c3ccccc3S7)nc3c2sc2ccccc23)cc1 ; c1ccc(-c2nc(-c3ccccc3-c3cccc4c3c3c5cccc6c5n(c3n4-c3ccccc3)-c3ccccc3S6)nc3sc4ccccc4c23)cc1]

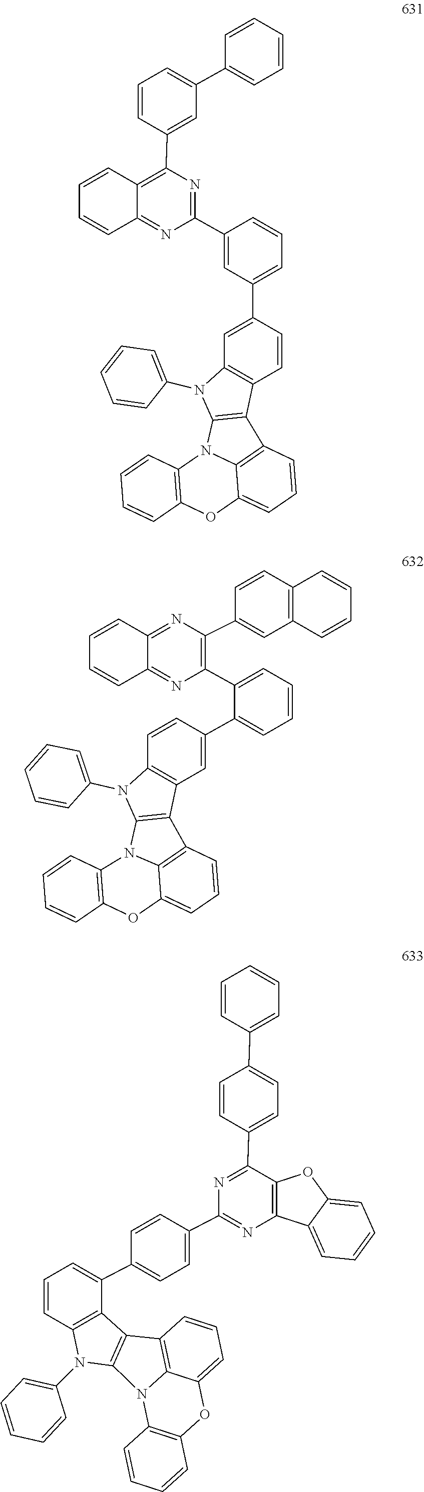 [SMILES: c1ccc(-c2cccc(-c3nc(-c4cccc(-c5ccc6c7c8cccc9c8n(c7n(-c7ccccc7)c6c5)-c5ccccc5O9)c4)nc4ccccc34)c2)cc1 ; c1ccc(-n2c3ccc(-c4ccccc4-c4nc5ccccc5nc4-c4ccc5ccccc5c4)cc3c3c4cccc5c4n(c32)-c2ccccc2O5)cc1 ; c1ccc(-c2ccc(-c3nc(-c4ccc(-c5cccc6c5c5c7cccc8c7n(c5n6-c5ccccc5)-c5ccccc5O8)cc4)nc4c3oc3ccccc34)cc2)cc1]

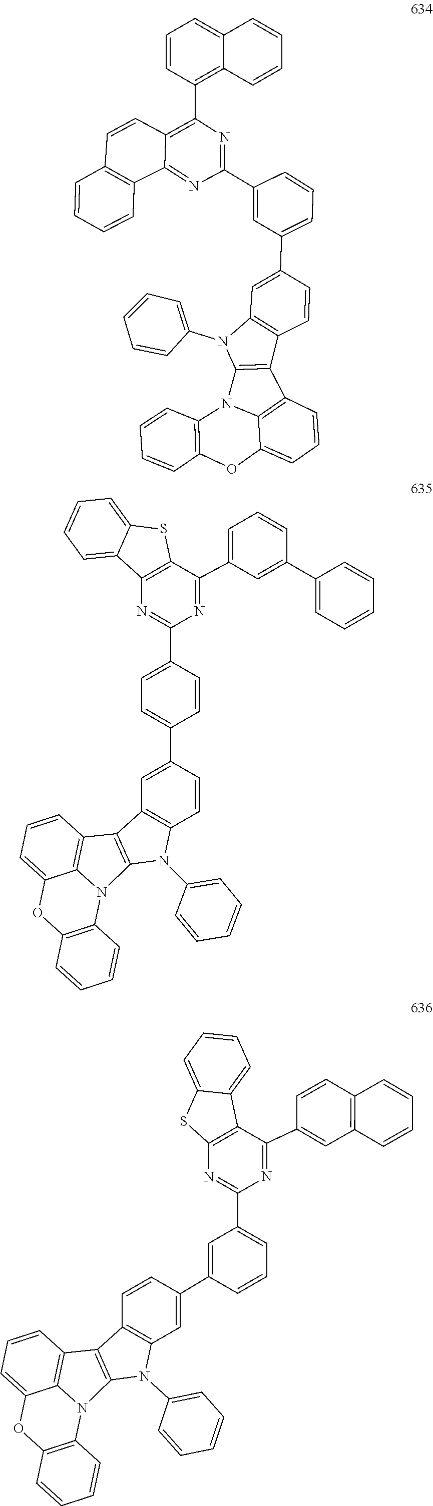 [SMILES: c1ccc(-n2c3cc(-c4cccc(-c5nc(-c6cccc7ccccc67)c6ccc7ccccc7c6n5)c4)ccc3c3c4cccc5c4n(c32)-c2ccccc2O5)cc1 ; c1ccc(-c2cccc(-c3nc(-c4ccc(-c5ccc6c(c5)c5c7cccc8c7n(c5n6-c5ccccc5)-c5ccccc5O8)cc4)nc4c3sc3ccccc34)c2)cc1 ; c1ccc(-n2c3cc(-c4cccc(-c5nc(-c6ccc7ccccc7c6)c6c(n5)sc5ccccc56)c4)ccc3c3c4cccc5c4n(c32)-c2ccccc2O5)cc1]

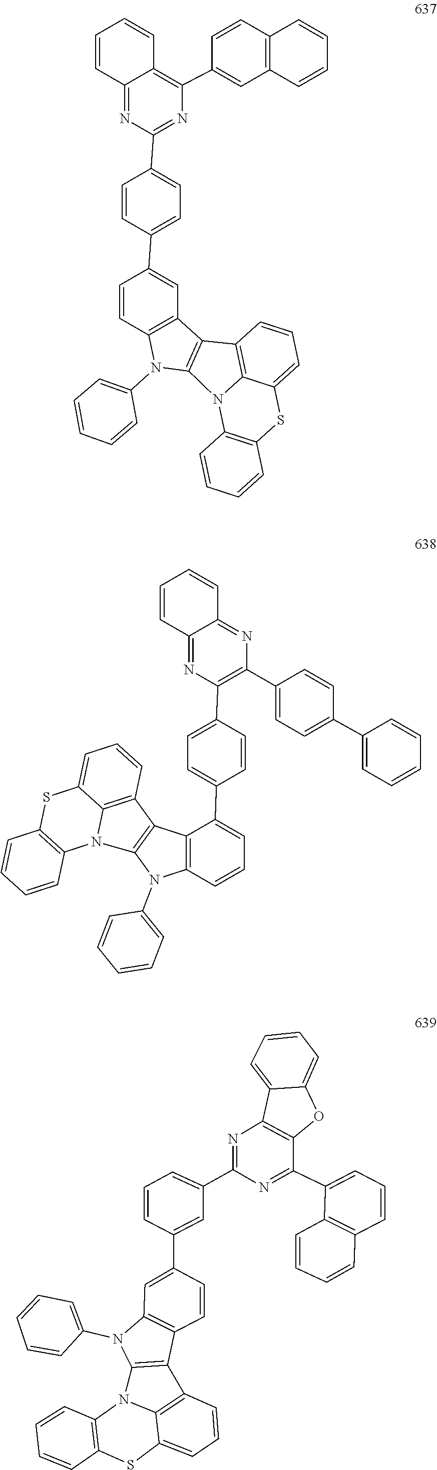 [SMILES: c1ccc(-n2c3ccc(-c4ccc(-c5nc(-c6ccc7ccccc7c6)c6ccccc6n5)cc4)cc3c3c4cccc5c4n(c32)-c2ccccc2S5)cc1 ; c1ccc(-c2ccc(-c3nc4ccccc4nc3-c3ccc(-c4cccc5c4c4c6cccc7c6n(c4n5-c4ccccc4)-c4ccccc4S7)cc3)cc2)cc1 ; c1ccc(-n2c3cc(-c4cccc(-c5nc(-c6cccc7ccccc67)c6oc7ccccc7c6n5)c4)ccc3c3c4cccc5c4n(c32)-c2ccccc2S5)cc1]

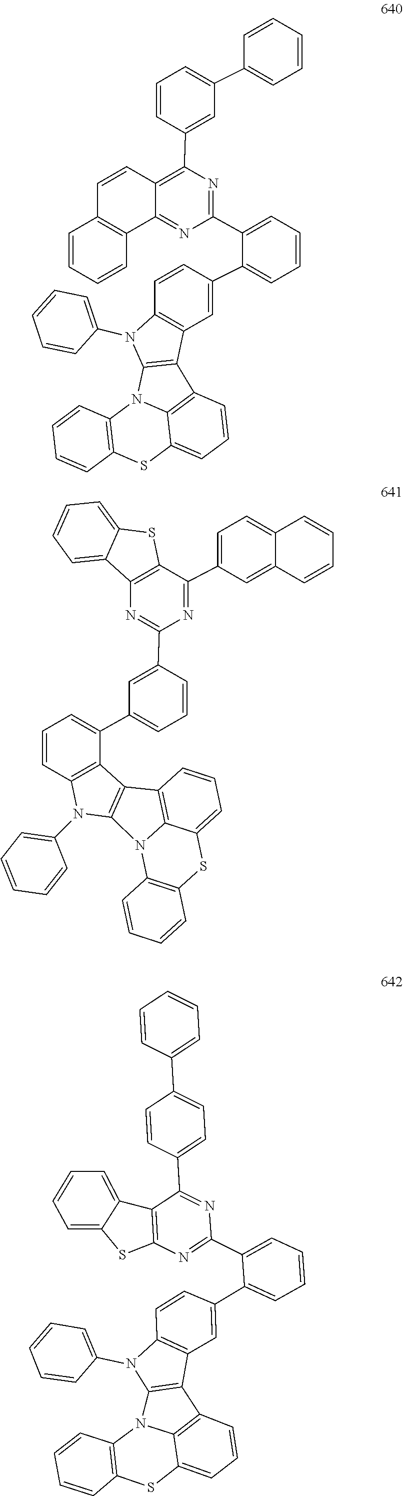

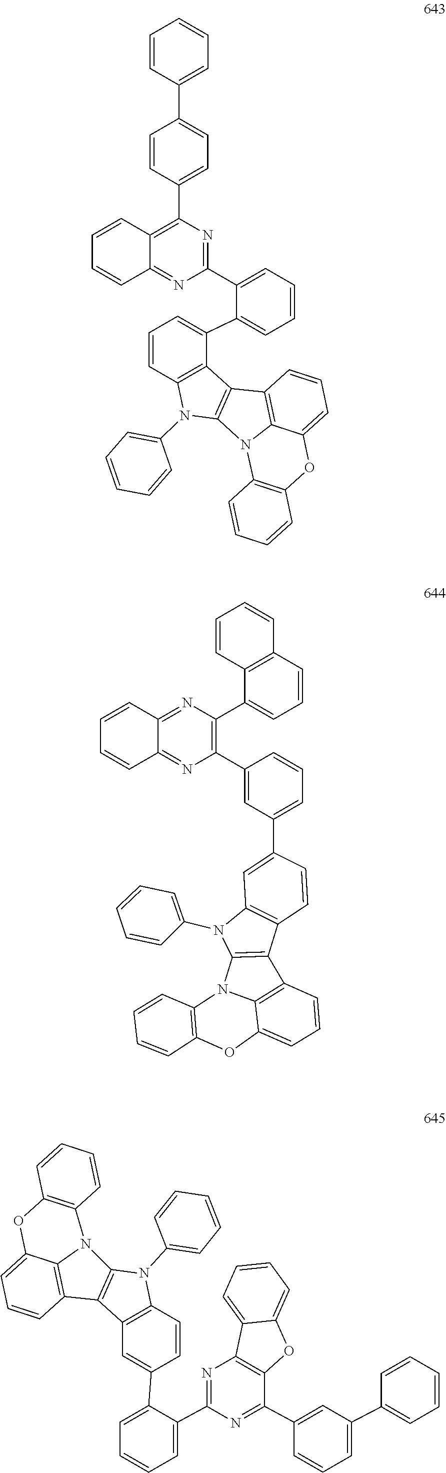 [SMILES: c1ccc(-c2ccc(-c3nc(-c4ccccc4-c4cccc5c4c4c6cccc7c6n(c4n5-c4ccccc4)-c4ccccc4O7)nc4ccccc34)cc2)cc1 ; c1ccc(-n2c3cc(-c4cccc(-c5nc6ccccc6nc5-c5cccc6ccccc56)c4)ccc3c3c4cccc5c4n(c32)-c2ccccc2O5)cc1 ; c1ccc(-c2cccc(-c3nc(-c4ccccc4-c4ccc5c(c4)c4c6cccc7c6n(c4n5-c4ccccc4)-c4ccccc4O7)nc4c3oc3ccccc34)c2)cc1]

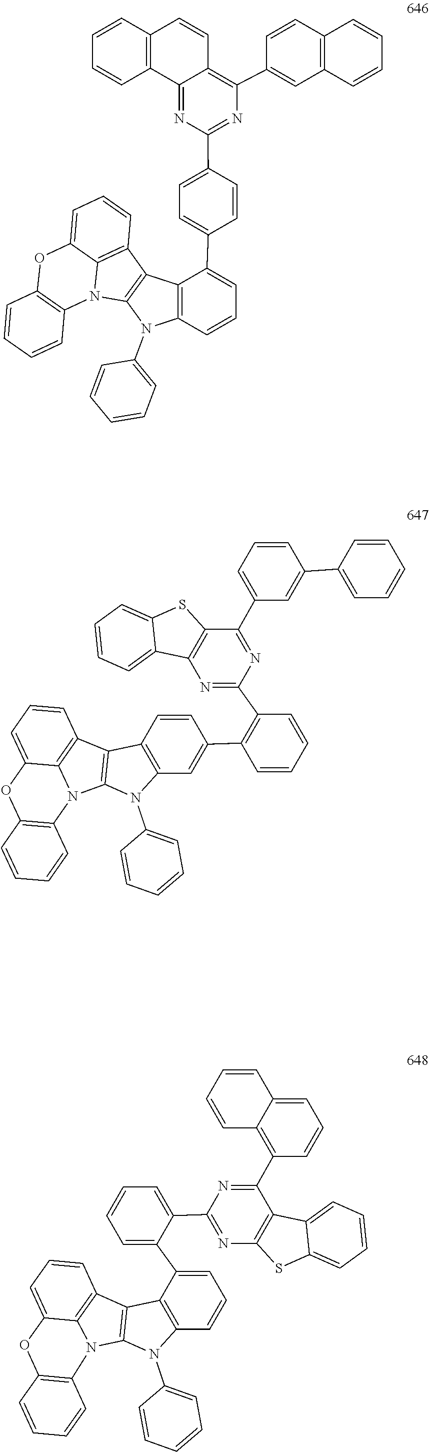 [SMILES: c1ccc(-n2c3cccc(-c4ccc(-c5nc(-c6ccc7ccccc7c6)c6ccc7ccccc7c6n5)cc4)c3c3c4cccc5c4n(c32)-c2ccccc2O5)cc1 ; c1ccc(-c2cccc(-c3nc(-c4ccccc4-c4ccc5c6c7cccc8c7n(c6n(-c6ccccc6)c5c4)-c4ccccc4O8)nc4c3sc3ccccc34)c2)cc1 ; c1ccc(-n2c3cccc(-c4ccccc4-c4nc(-c5cccc6ccccc56)c5c(n4)sc4ccccc45)c3c3c4cccc5c4n(c32)-c2ccccc2O5)cc1]

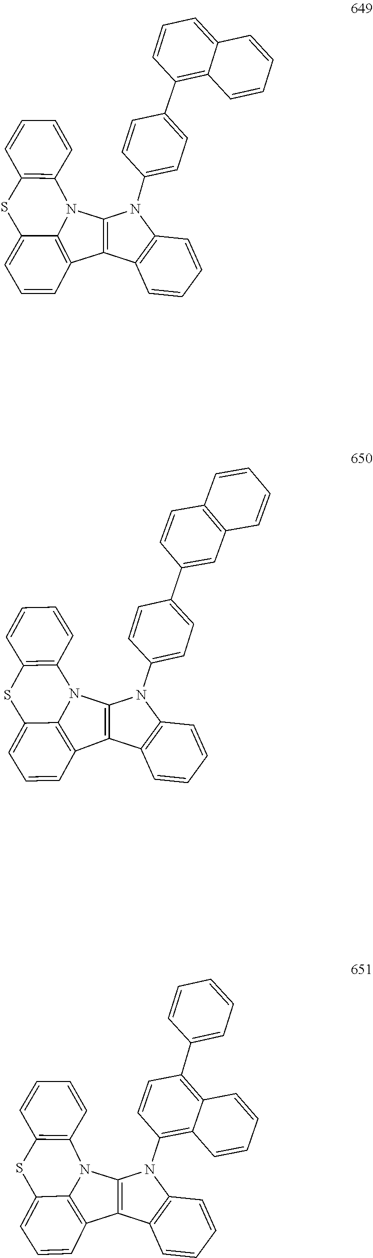 [SMILES: c1ccc2c(c1)Sc1cccc3c4c5ccccc5n(-c5ccc(-c6cccc7ccccc67)cc5)c4n-2c13 ; c1ccc2c(c1)Sc1cccc3c4c5ccccc5n(-c5ccc(-c6ccc7ccccc7c6)cc5)c4n-2c13 ; c1ccc(-c2ccc(-n3c4ccccc4c4c5cccc6c5n(c43)-c3ccccc3S6)c3ccccc23)cc1]

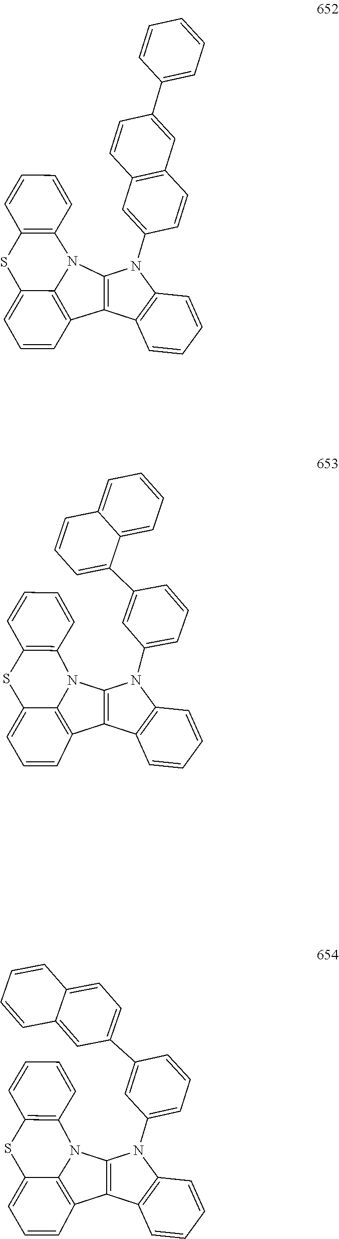 [SMILES: c1ccc(-c2ccc3cc(-n4c5ccccc5c5c6cccc7c6n(c54)-c4ccccc4S7)ccc3c2)cc1 ; c1cc(-c2cccc3ccccc23)cc(-n2c3ccccc3c3c4cccc5c4n(c32)-c2ccccc2S5)c1 ; c1cc(-c2ccc3ccccc3c2)cc(-n2c3ccccc3c3c4cccc5c4n(c32)-c2ccccc2S5)c1]

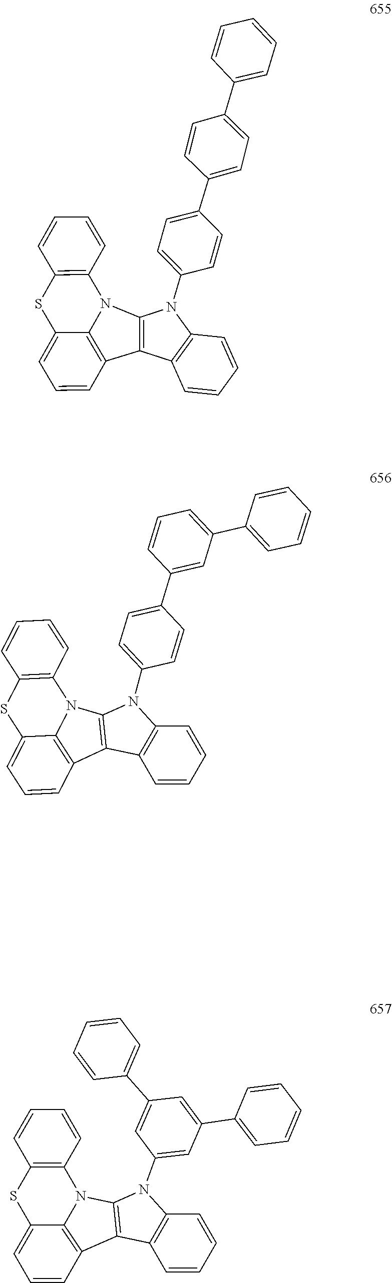 [SMILES: c1ccc(-c2ccc(-c3ccc(-n4c5ccccc5c5c6cccc7c6n(c54)-c4ccccc4S7)cc3)cc2)cc1 ; c1ccc(-c2cccc(-c3ccc(-n4c5ccccc5c5c6cccc7c6n(c54)-c4ccccc4S7)cc3)c2)cc1 ; c1ccc(-c2cc(-c3ccccc3)cc(-n3c4ccccc4c4c5cccc6c5n(c43)-c3ccccc3S6)c2)cc1]

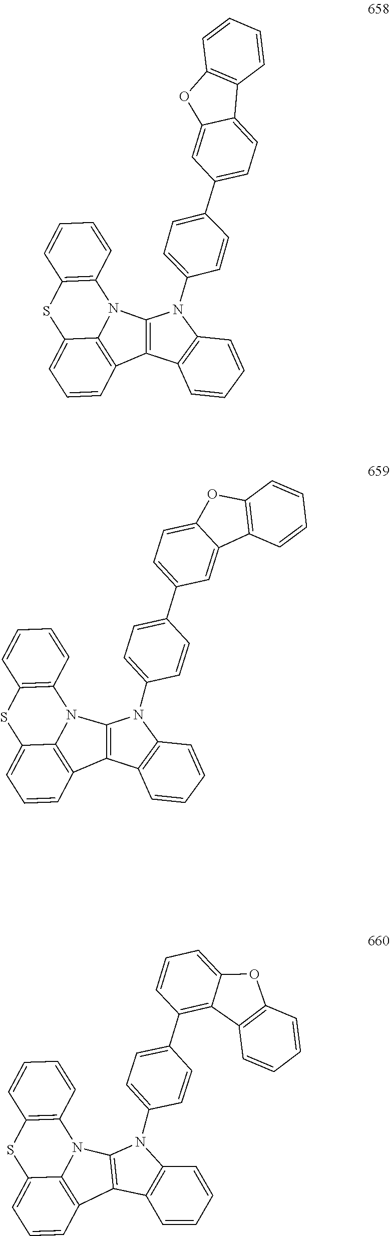 [SMILES: c1ccc2c(c1)Sc1cccc3c4c5ccccc5n(-c5ccc(-c6ccc7c(c6)oc6ccccc67)cc5)c4n-2c13 ; c1ccc2c(c1)Sc1cccc3c4c5ccccc5n(-c5ccc(-c6ccc7oc8ccccc8c7c6)cc5)c4n-2c13 ; c1ccc2c(c1)Sc1cccc3c4c5ccccc5n(-c5ccc(-c6cccc7oc8ccccc8c67)cc5)c4n-2c13]

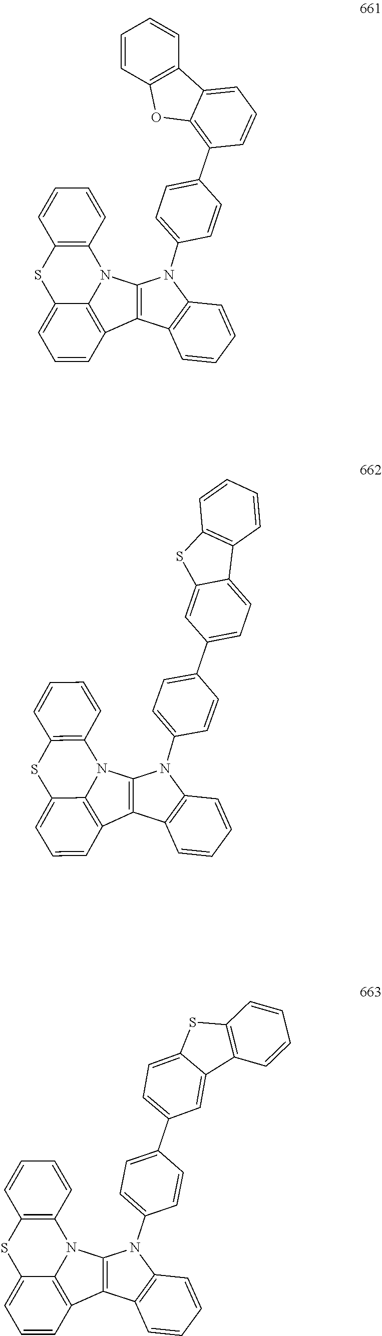 [SMILES: c1ccc2c(c1)Sc1cccc3c4c5ccccc5n(-c5ccc(-c6cccc7c6oc6ccccc67)cc5)c4n-2c13 ; c1ccc2c(c1)Sc1cccc3c4c5ccccc5n(-c5ccc(-c6ccc7c(c6)sc6ccccc67)cc5)c4n-2c13 ; c1ccc2c(c1)Sc1cccc3c4c5ccccc5n(-c5ccc(-c6ccc7sc8ccccc8c7c6)cc5)c4n-2c13]

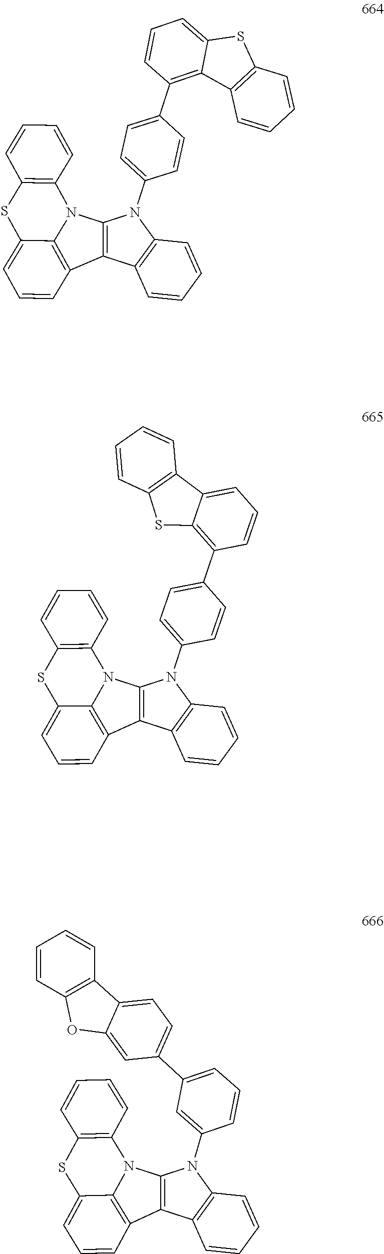 [SMILES: c1ccc2c(c1)Sc1cccc3c4c5ccccc5n(-c5ccc(-c6cccc7sc8ccccc8c67)cc5)c4n-2c13 ; c1ccc2c(c1)Sc1cccc3c4c5ccccc5n(-c5ccc(-c6cccc7c6sc6ccccc67)cc5)c4n-2c13 ; c1cc(-c2ccc3c(c2)oc2ccccc23)cc(-n2c3ccccc3c3c4cccc5c4n(c32)-c2ccccc2S5)c1]

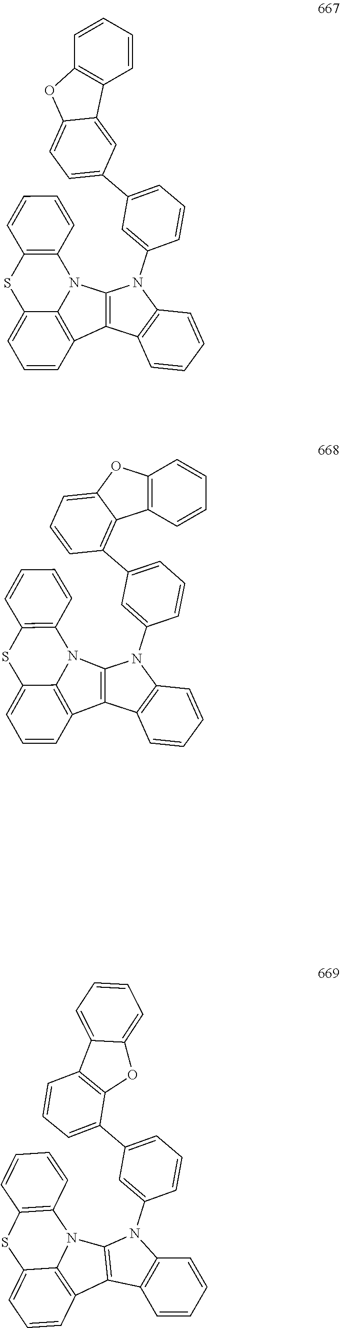 [SMILES: c1cc(-c2ccc3oc4ccccc4c3c2)cc(-n2c3ccccc3c3c4cccc5c4n(c32)-c2ccccc2S5)c1 ; c1cc(-c2cccc3oc4ccccc4c23)cc(-n2c3ccccc3c3c4cccc5c4n(c32)-c2ccccc2S5)c1 ; c1cc(-c2cccc3c2oc2ccccc23)cc(-n2c3ccccc3c3c4cccc5c4n(c32)-c2ccccc2S5)c1]

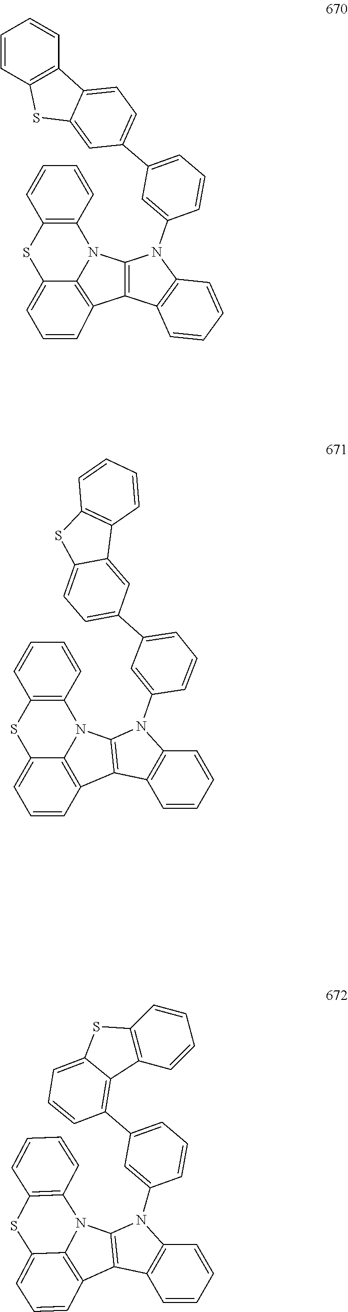 [SMILES: c1cc(-c2ccc3c(c2)sc2ccccc23)cc(-n2c3ccccc3c3c4cccc5c4n(c32)-c2ccccc2S5)c1 ; c1cc(-c2ccc3sc4ccccc4c3c2)cc(-n2c3ccccc3c3c4cccc5c4n(c32)-c2ccccc2S5)c1 ; c1cc(-c2cccc3sc4ccccc4c23)cc(-n2c3ccccc3c3c4cccc5c4n(c32)-c2ccccc2S5)c1]

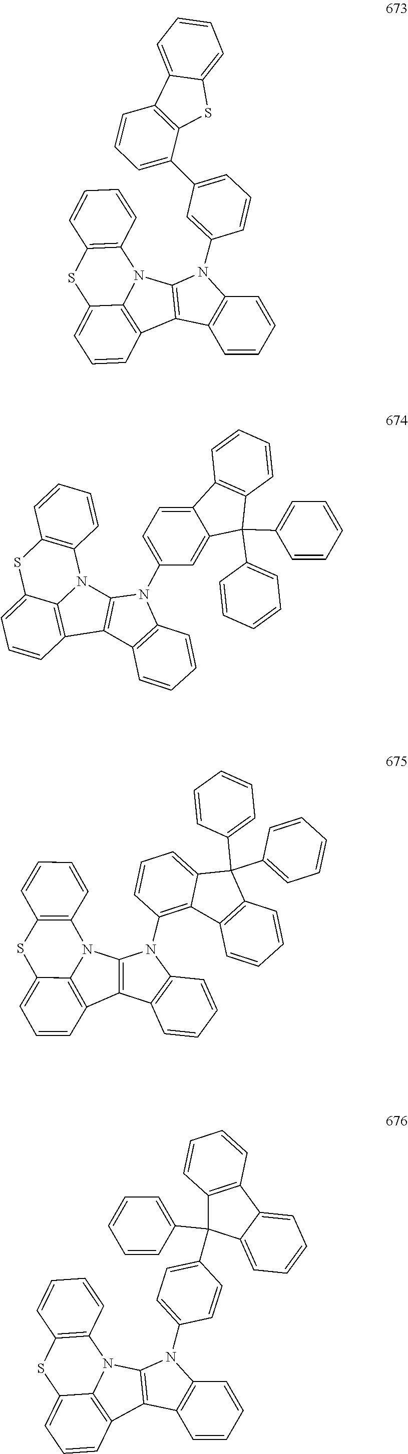 [SMILES: c1cc(-c2cccc3c2sc2ccccc23)cc(-n2c3ccccc3c3c4cccc5c4n(c32)-c2ccccc2S5)c1 ; c1ccc(C2(c3ccccc3)c3ccccc3-c3ccc(-n4c5ccccc5c5c6cccc7c6n(c54)-c4ccccc4S7)cc32)cc1 ; c1ccc(C2(c3ccccc3)c3ccccc3-c3c(-n4c5ccccc5c5c6cccc7c6n(c54)-c4ccccc4S7)cccc32)cc1 ; c1ccc(C2(c3ccc(-n4c5ccccc5c5c6cccc7c6n(c54)-c4ccccc4S7)cc3)c3ccccc3-c3ccccc32)cc1]

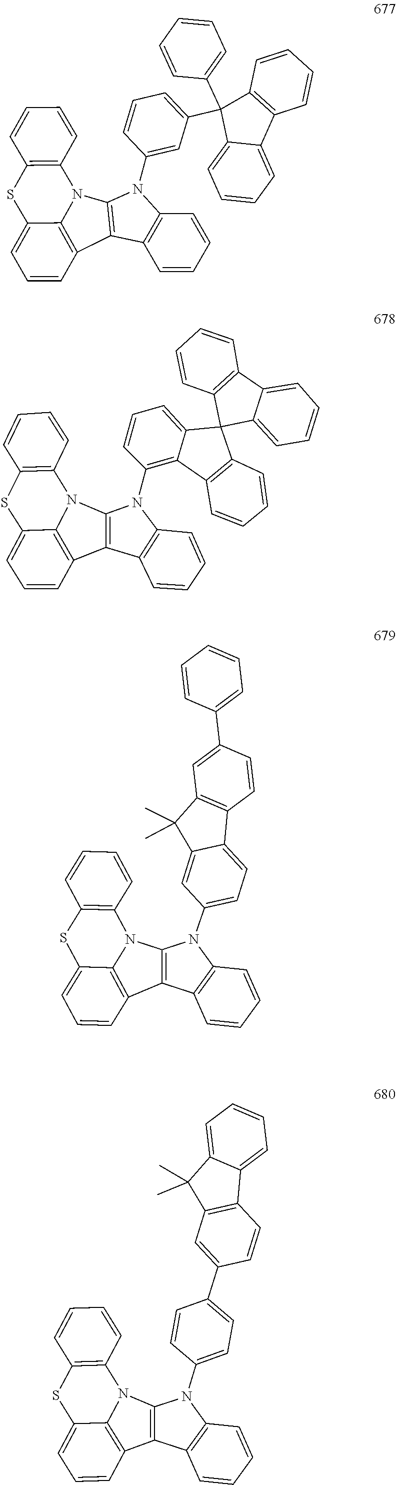 [SMILES: c1ccc(C2(c3cccc(-n4c5ccccc5c5c6cccc7c6n(c54)-c4ccccc4S7)c3)c3ccccc3-c3ccccc32)cc1 ; c1ccc2c(c1)Sc1cccc3c4c5ccccc5n(-c5cccc6c5-c5ccccc5C65c6ccccc6-c6ccccc65)c4n-2c13 ; CC1(C)c2cc(-c3ccccc3)ccc2-c2ccc(-n3c4ccccc4c4c5cccc6c5n(c43)-c3ccccc3S6)cc21 ; CC1(C)c2ccccc2-c2ccc(-c3ccc(-n4c5ccccc5c5c6cccc7c6n(c54)-c4ccccc4S7)cc3)cc21]

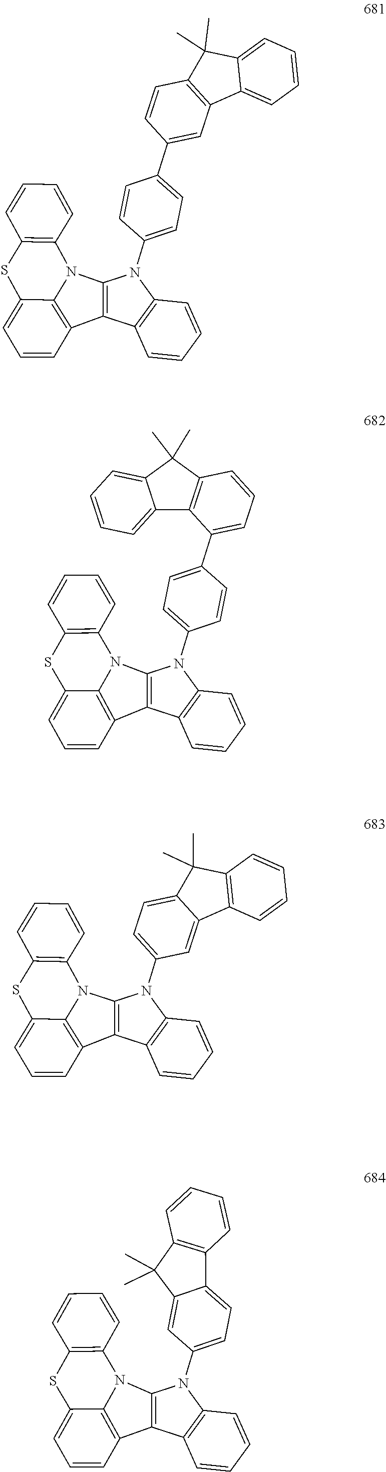 [SMILES: CC1(C)c2ccccc2-c2cc(-c3ccc(-n4c5ccccc5c5c6cccc7c6n(c54)-c4ccccc4S7)cc3)ccc21 ; CC1(C)c2ccccc2-c2c(-c3ccc(-n4c5ccccc5c5c6cccc7c6n(c54)-c4ccccc4S7)cc3)cccc21 ; CC1(C)c2ccccc2-c2cc(-n3c4ccccc4c4c5cccc6c5n(c43)-c3ccccc3S6)ccc21 ; CC1(C)c2ccccc2-c2ccc(-n3c4ccccc4c4c5cccc6c5n(c43)-c3ccccc3S6)cc21]

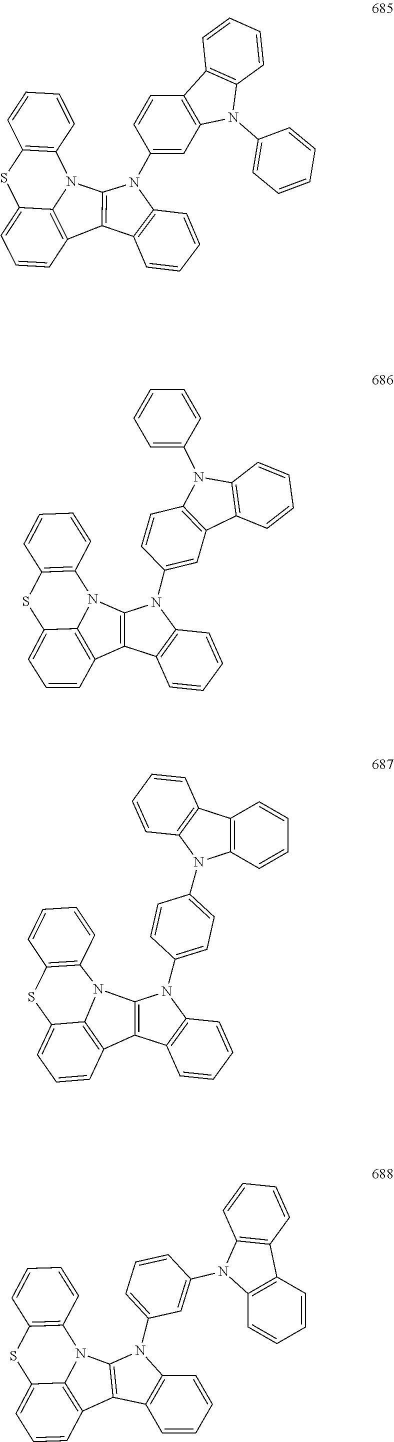 [SMILES: c1ccc(-n2c3ccccc3c3ccc(-n4c5ccccc5c5c6cccc7c6n(c54)-c4ccccc4S7)cc32)cc1 ; c1ccc(-n2c3ccccc3c3cc(-n4c5ccccc5c5c6cccc7c6n(c54)-c4ccccc4S7)ccc32)cc1 ; c1ccc2c(c1)Sc1cccc3c4c5ccccc5n(-c5ccc(-n6c7ccccc7c7ccccc76)cc5)c4n-2c13 ; c1cc(-n2c3ccccc3c3ccccc32)cc(-n2c3ccccc3c3c4cccc5c4n(c32)-c2ccccc2S5)c1]

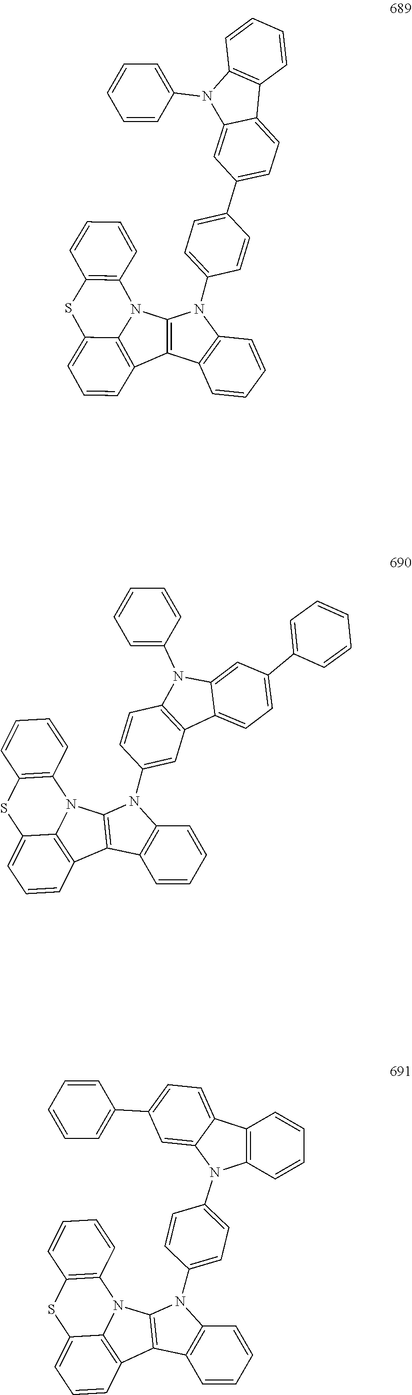 [SMILES: c1ccc(-n2c3ccccc3c3ccc(-c4ccc(-n5c6ccccc6c6c7cccc8c7n(c65)-c5ccccc5S8)cc4)cc32)cc1 ; c1ccc(-c2ccc3c4cc(-n5c6ccccc6c6c7cccc8c7n(c65)-c5ccccc5S8)ccc4n(-c4ccccc4)c3c2)cc1 ; c1ccc(-c2ccc3c4ccccc4n(-c4ccc(-n5c6ccccc6c6c7cccc8c7n(c65)-c5ccccc5S8)cc4)c3c2)cc1]

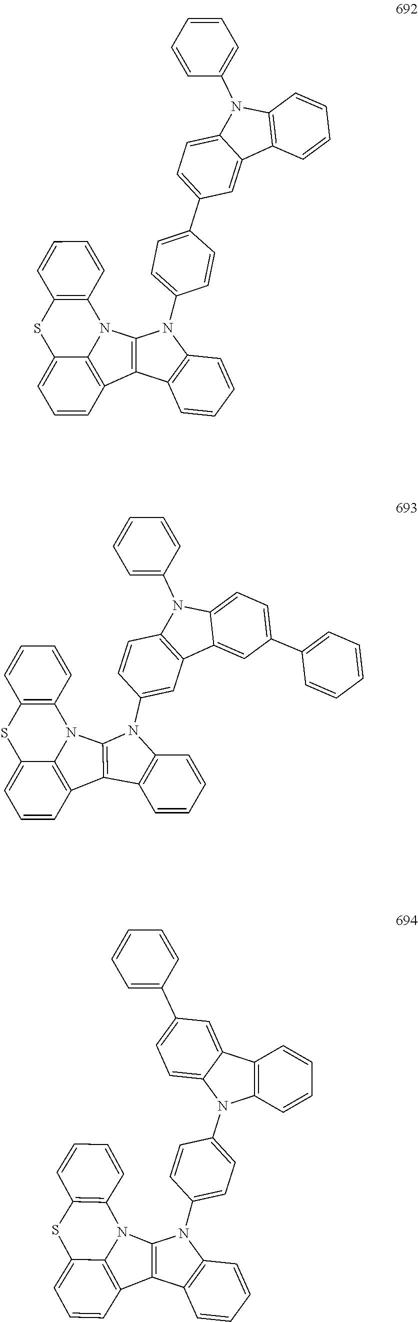 [SMILES: c1ccc(-n2c3ccccc3c3cc(-c4ccc(-n5c6ccccc6c6c7cccc8c7n(c65)-c5ccccc5S8)cc4)ccc32)cc1 ; c1ccc(-c2ccc3c(c2)c2cc(-n4c5ccccc5c5c6cccc7c6n(c54)-c4ccccc4S7)ccc2n3-c2ccccc2)cc1 ; c1ccc(-c2ccc3c(c2)c2ccccc2n3-c2ccc(-n3c4ccccc4c4c5cccc6c5n(c43)-c3ccccc3S6)cc2)cc1]

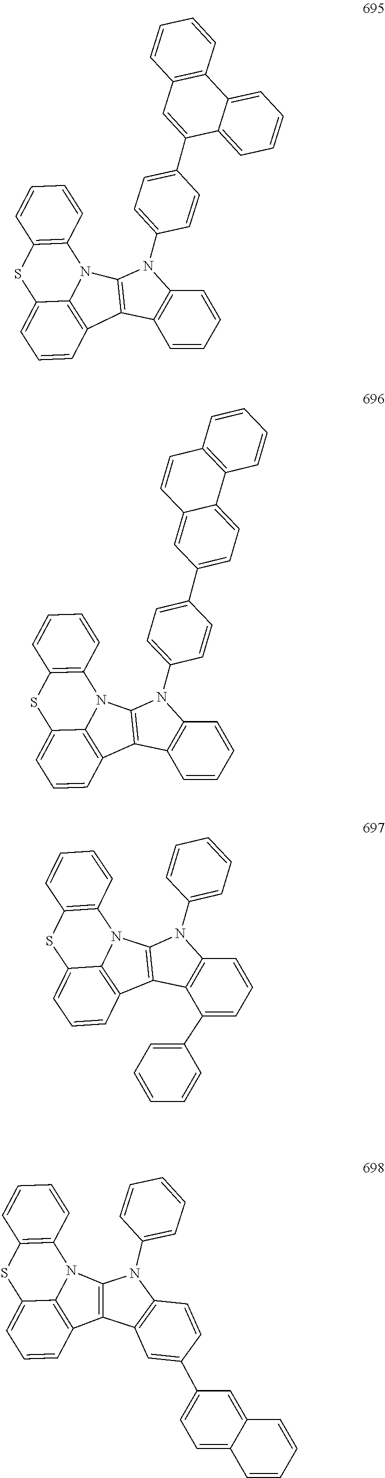 [SMILES: c1ccc2c(c1)Sc1cccc3c4c5ccccc5n(-c5ccc(-c6cc7ccccc7c7ccccc67)cc5)c4n-2c13 ; c1ccc2c(c1)Sc1cccc3c4c5ccccc5n(-c5ccc(-c6ccc7c(ccc8ccccc87)c6)cc5)c4n-2c13 ; c1ccc(-c2cccc3c2c2c4cccc5c4n(c2n3-c2ccccc2)-c2ccccc2S5)cc1 ; c1ccc(-n2c3ccc(-c4ccc5ccccc5c4)cc3c3c4cccc5c4n(c32)-c2ccccc2S5)cc1]

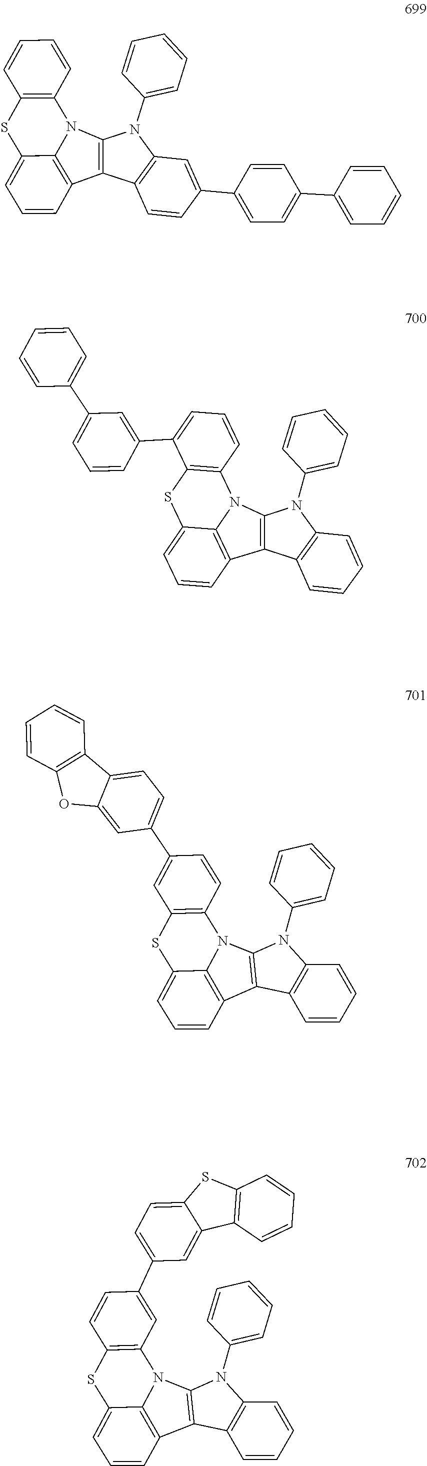 [SMILES: c1ccc(-c2ccc(-c3ccc4c5c6cccc7c6n(c5n(-c5ccccc5)c4c3)-c3ccccc3S7)cc2)cc1 ; c1ccc(-c2cccc(-c3cccc4c3Sc3cccc5c6c7ccccc7n(-c7ccccc7)c6n-4c35)c2)cc1 ; c1ccc(-n2c3ccccc3c3c4cccc5c4n(c32)-c2ccc(-c3ccc4c(c3)oc3ccccc34)cc2S5)cc1 ; c1ccc(-n2c3ccccc3c3c4cccc5c4n(c32)-c2cc(-c3ccc4sc6ccccc6c4c3)ccc2S5)cc1]

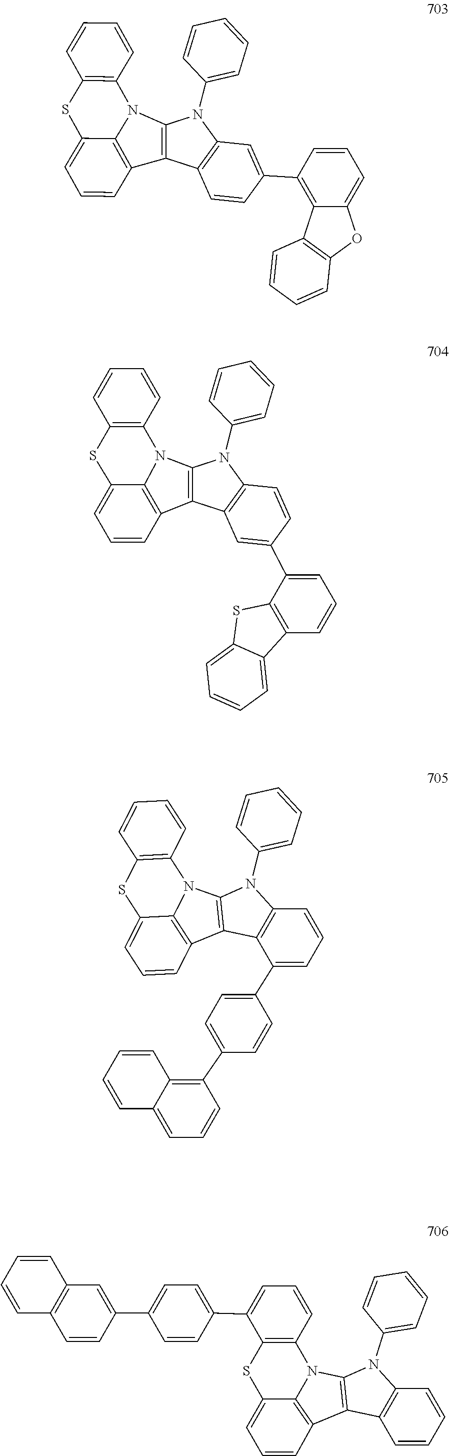 [SMILES: c1ccc(-n2c3cc(-c4cccc5oc6ccccc6c45)ccc3c3c4cccc5c4n(c32)-c2ccccc2S5)cc1 ; c1ccc(-n2c3ccc(-c4cccc5c4sc4ccccc45)cc3c3c4cccc5c4n(c32)-c2ccccc2S5)cc1 ; c1ccc(-n2c3cccc(-c4ccc(-c5cccc6ccccc56)cc4)c3c3c4cccc5c4n(c32)-c2ccccc2S5)cc1 ; c1ccc(-n2c3ccccc3c3c4cccc5c4n(c32)-c2cccc(-c3ccc(-c4ccc6ccccc6c4)cc3)c2S5)cc1]

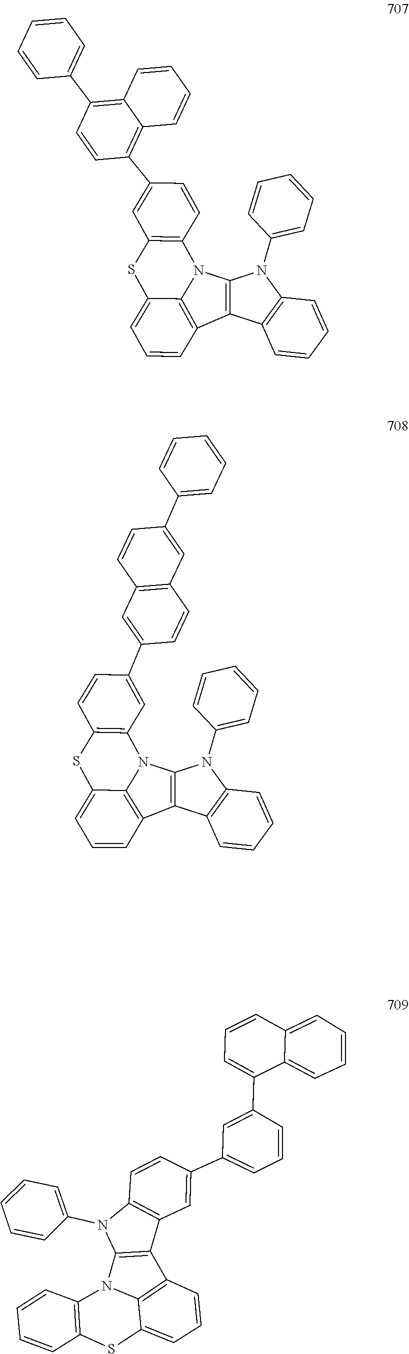 [SMILES: c1ccc(-c2ccc(-c3ccc4c(c3)Sc3cccc5c6c7ccccc7n(-c7ccccc7)c6n-4c35)c3ccccc23)cc1 ; c1ccc(-c2ccc3cc(-c4ccc5c(c4)-n4c6c(cccc6c6c7ccccc7n(-c7ccccc7)c64)S5)ccc3c2)cc1 ; c1ccc(-n2c3ccc(-c4cccc(-c5cccc6ccccc56)c4)cc3c3c4cccc5c4n(c32)-c2ccccc2S5)cc1]

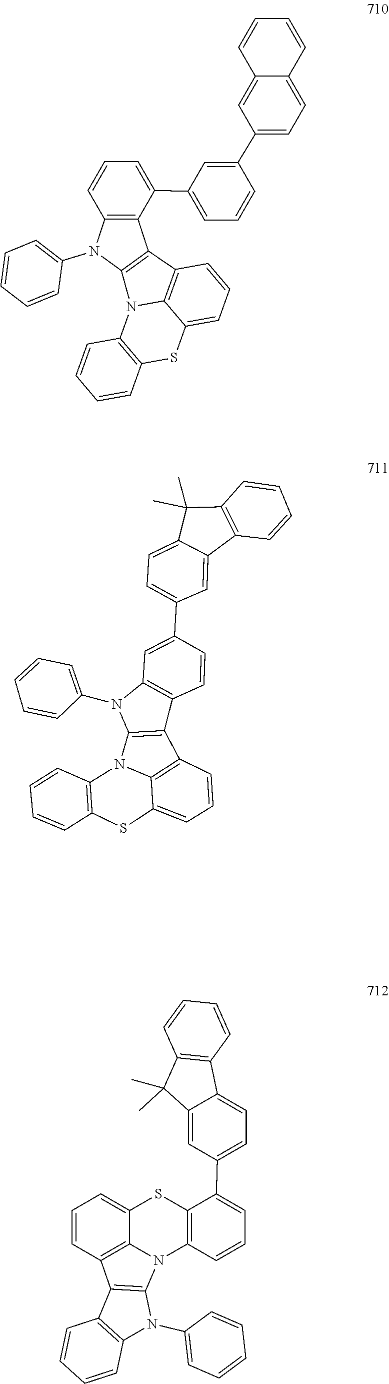 [SMILES: c1ccc(-n2c3cccc(-c4cccc(-c5ccc6ccccc6c5)c4)c3c3c4cccc5c4n(c32)-c2ccccc2S5)cc1 ; CC1(C)c2ccccc2-c2cc(-c3ccc4c5c6cccc7c6n(c5n(-c5ccccc5)c4c3)-c3ccccc3S7)ccc21 ; CC1(C)c2ccccc2-c2ccc(-c3cccc4c3Sc3cccc5c6c7ccccc7n(-c7ccccc7)c6n-4c35)cc21]

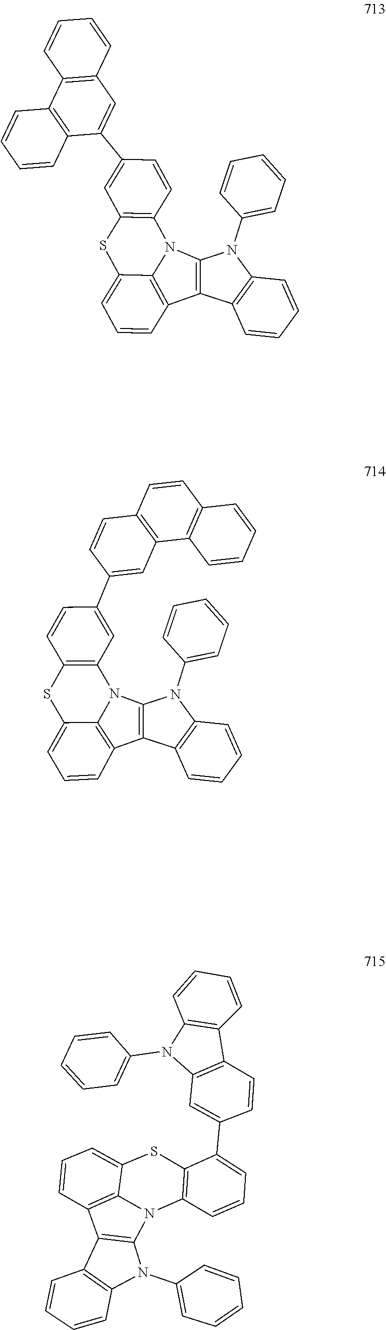 [SMILES: c1ccc(-n2c3ccccc3c3c4cccc5c4n(c32)-c2ccc(-c3cc4ccccc4c4ccccc34)cc2S5)cc1 ; c1ccc(-n2c3ccccc3c3c4cccc5c4n(c32)-c2cc(-c3ccc4ccc6ccccc6c4c3)ccc2S5)cc1 ; c1ccc(-n2c3ccccc3c3ccc(-c4cccc5c4Sc4cccc6c7c8ccccc8n(-c8ccccc8)c7n-5c46)cc32)cc1]

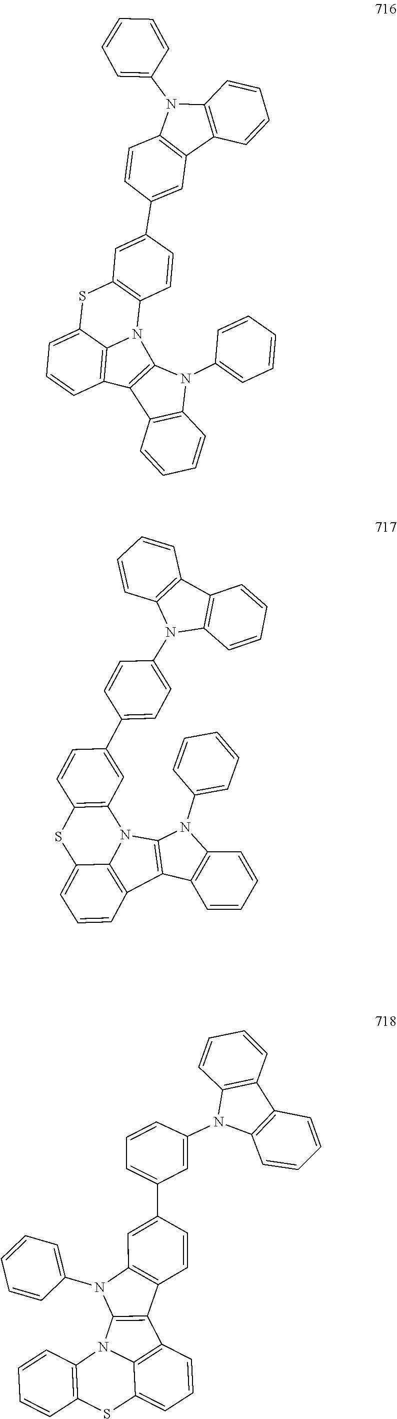 [SMILES: c1ccc(-n2c3ccccc3c3cc(-c4ccc5c(c4)Sc4cccc6c7c8ccccc8n(-c8ccccc8)c7n-5c46)ccc32)cc1 ; c1ccc(-n2c3ccccc3c3c4cccc5c4n(c32)-c2cc(-c3ccc(-n4c6ccccc6c6ccccc64)cc3)ccc2S5)cc1 ; c1ccc(-n2c3cc(-c4cccc(-n5c6ccccc6c6ccccc65)c4)ccc3c3c4cccc5c4n(c32)-c2ccccc2S5)cc1]

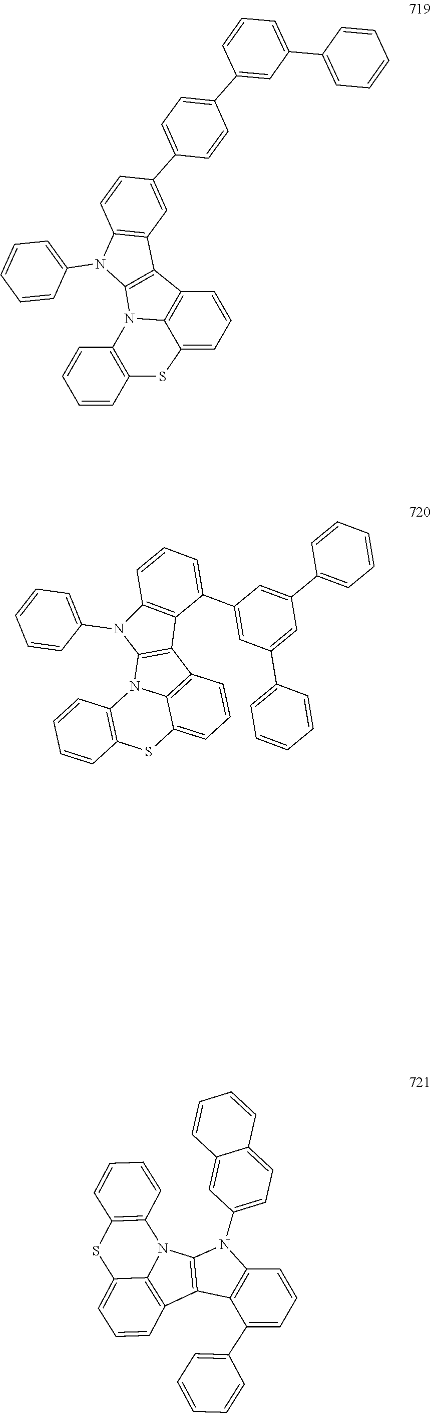 [SMILES: c1ccc(-c2cccc(-c3ccc(-c4ccc5c(c4)c4c6cccc7c6n(c4n5-c4ccccc4)-c4ccccc4S7)cc3)c2)cc1 ; c1ccc(-c2cc(-c3ccccc3)cc(-c3cccc4c3c3c5cccc6c5n(c3n4-c3ccccc3)-c3ccccc3S6)c2)cc1 ; c1ccc(-c2cccc3c2c2c4cccc5c4n(c2n3-c2ccc3ccccc3c2)-c2ccccc2S5)cc1]

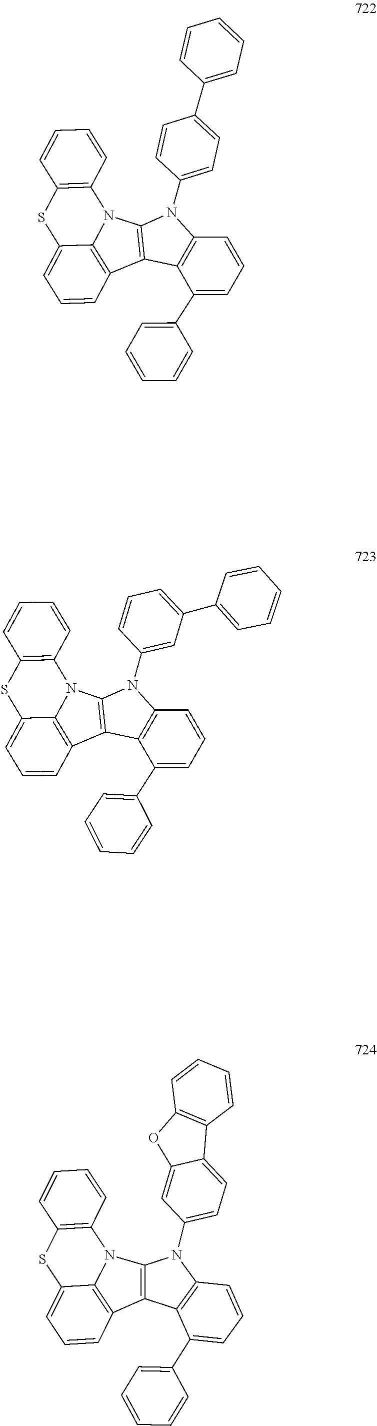 [SMILES: c1ccc(-c2ccc(-n3c4cccc(-c5ccccc5)c4c4c5cccc6c5n(c43)-c3ccccc3S6)cc2)cc1 ; c1ccc(-c2cccc(-n3c4cccc(-c5ccccc5)c4c4c5cccc6c5n(c43)-c3ccccc3S6)c2)cc1 ; c1ccc(-c2cccc3c2c2c4cccc5c4n(c2n3-c2ccc3c(c2)oc2ccccc23)-c2ccccc2S5)cc1]

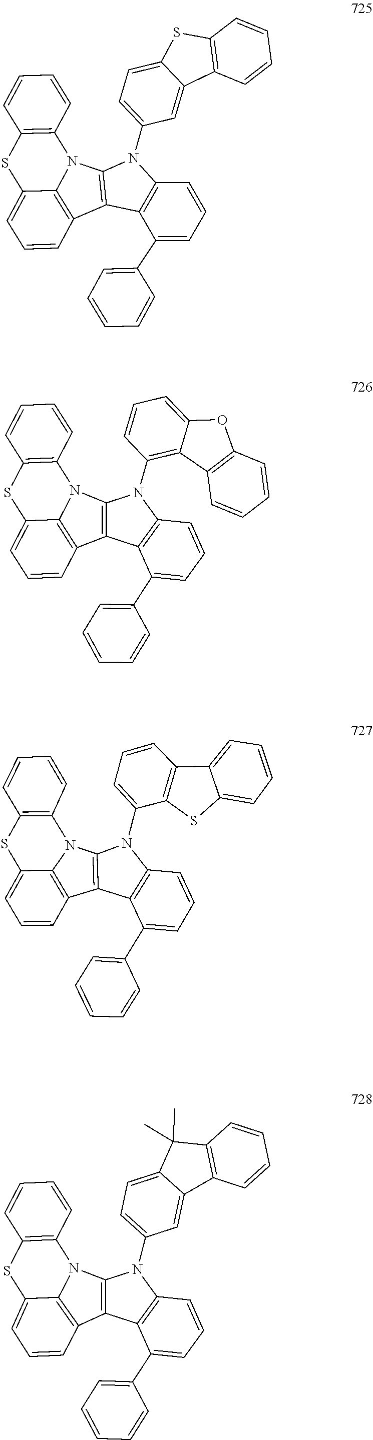 [SMILES: c1ccc(-c2cccc3c2c2c4cccc5c4n(c2n3-c2ccc3sc4ccccc4c3c2)-c2ccccc2S5)cc1 ; c1ccc(-c2cccc3c2c2c4cccc5c4n(c2n3-c2cccc3oc4ccccc4c23)-c2ccccc2S5)cc1 ; c1ccc(-c2cccc3c2c2c4cccc5c4n(c2n3-c2cccc3c2sc2ccccc23)-c2ccccc2S5)cc1 ; CC1(C)c2ccccc2-c2cc(-n3c4cccc(-c5ccccc5)c4c4c5cccc6c5n(c43)-c3ccccc3S6)ccc21]

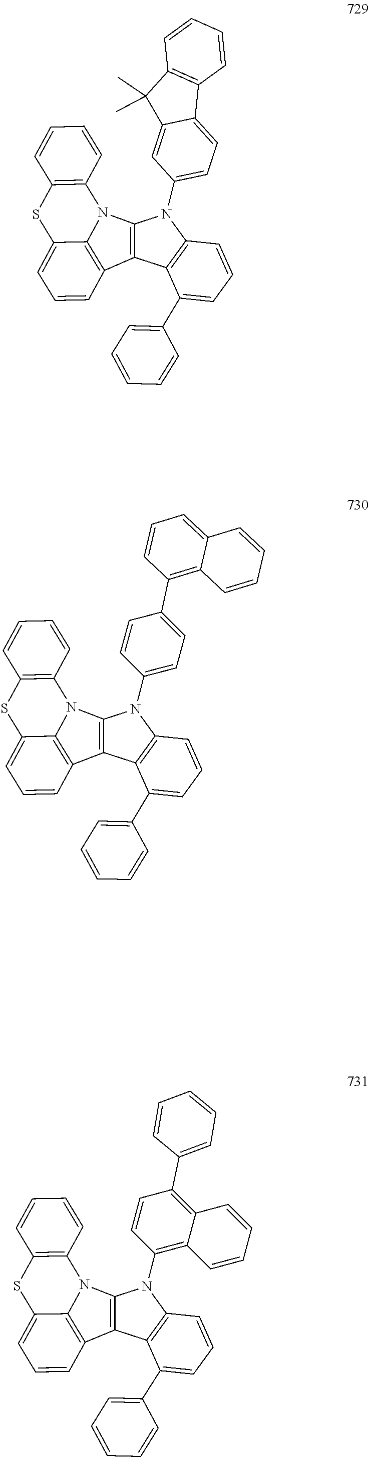 [SMILES: CC1(C)c2ccccc2-c2ccc(-n3c4cccc(-c5ccccc5)c4c4c5cccc6c5n(c43)-c3ccccc3S6)cc21 ; c1ccc(-c2cccc3c2c2c4cccc5c4n(c2n3-c2ccc(-c3cccc4ccccc34)cc2)-c2ccccc2S5)cc1 ; c1ccc(-c2ccc(-n3c4cccc(-c5ccccc5)c4c4c5cccc6c5n(c43)-c3ccccc3S6)c3ccccc23)cc1]

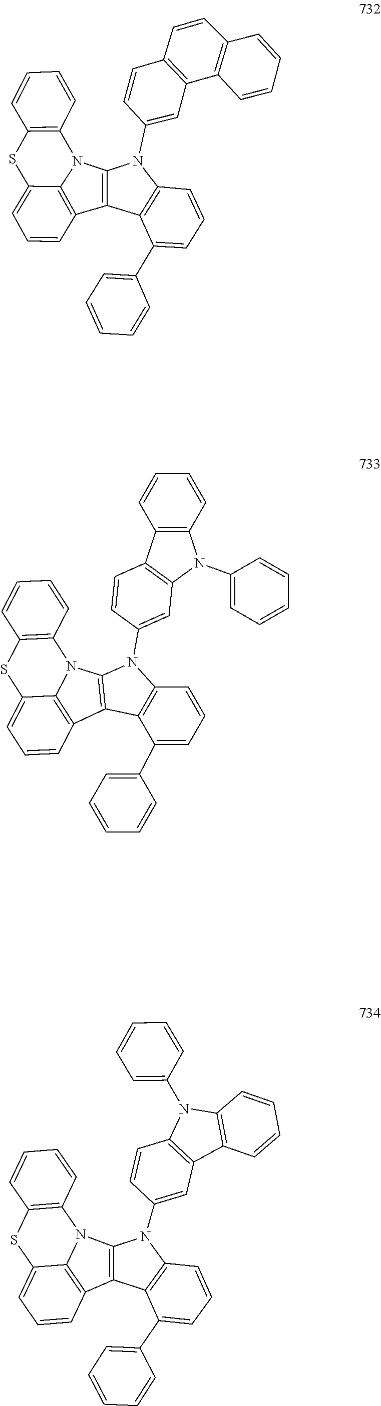 [SMILES: c1ccc(-c2cccc3c2c2c4cccc5c4n(c2n3-c2ccc3ccc4ccccc4c3c2)-c2ccccc2S5)cc1 ; c1ccc(-c2cccc3c2c2c4cccc5c4n(c2n3-c2ccc3c4ccccc4n(-c4ccccc4)c3c2)-c2ccccc2S5)cc1 ; c1ccc(-c2cccc3c2c2c4cccc5c4n(c2n3-c2ccc3c(c2)c2ccccc2n3-c2ccccc2)-c2ccccc2S5)cc1]

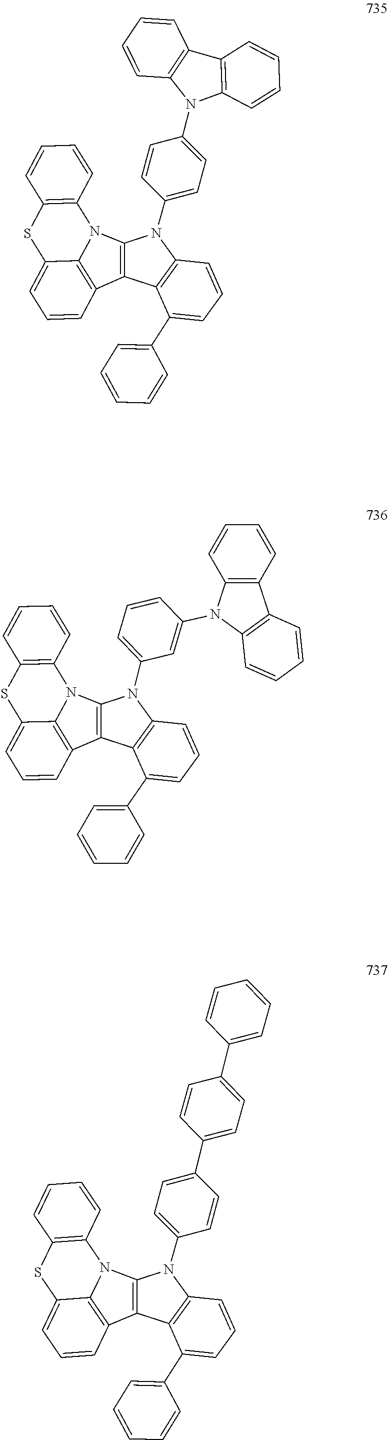 [SMILES: c1ccc(-c2cccc3c2c2c4cccc5c4n(c2n3-c2ccc(-n3c4ccccc4c4ccccc43)cc2)-c2ccccc2S5)cc1 ; c1ccc(-c2cccc3c2c2c4cccc5c4n(c2n3-c2cccc(-n3c4ccccc4c4ccccc43)c2)-c2ccccc2S5)cc1 ; c1ccc(-c2ccc(-c3ccc(-n4c5cccc(-c6ccccc6)c5c5c6cccc7c6n(c54)-c4ccccc4S7)cc3)cc2)cc1]

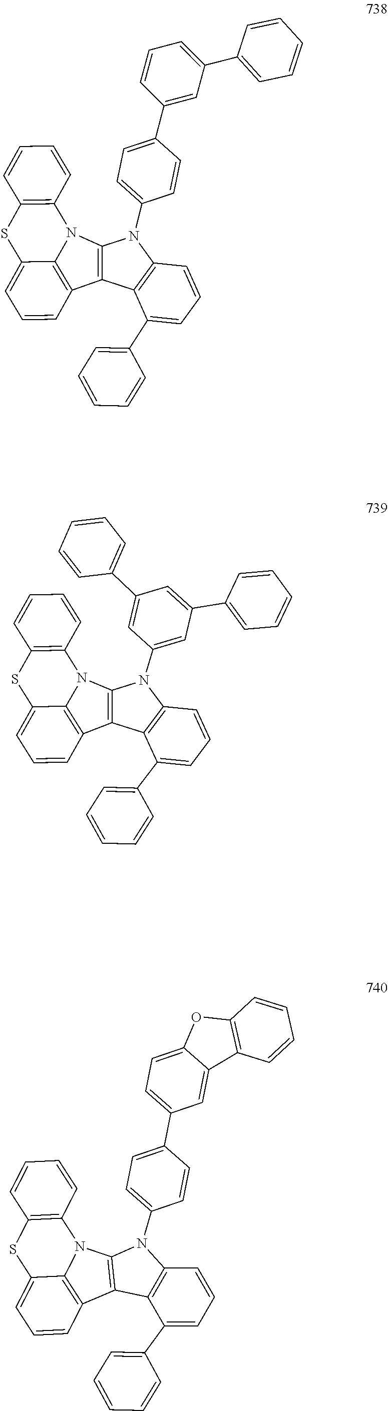 [SMILES: c1ccc(-c2cccc(-c3ccc(-n4c5cccc(-c6ccccc6)c5c5c6cccc7c6n(c54)-c4ccccc4S7)cc3)c2)cc1 ; c1ccc(-c2cc(-c3ccccc3)cc(-n3c4cccc(-c5ccccc5)c4c4c5cccc6c5n(c43)-c3ccccc3S6)c2)cc1 ; c1ccc(-c2cccc3c2c2c4cccc5c4n(c2n3-c2ccc(-c3ccc4oc6ccccc6c4c3)cc2)-c2ccccc2S5)cc1]

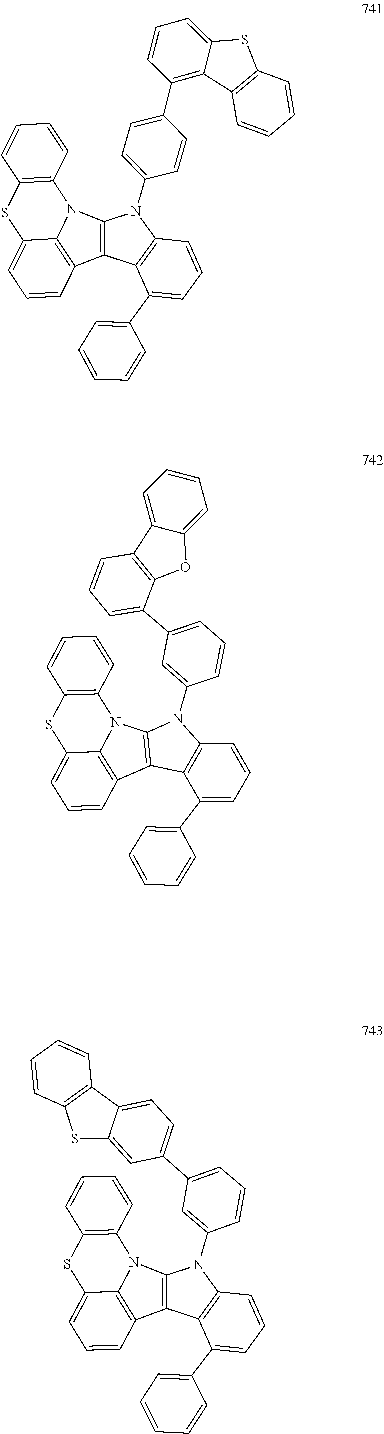 [SMILES: c1ccc(-c2cccc3c2c2c4cccc5c4n(c2n3-c2ccc(-c3cccc4sc6ccccc6c34)cc2)-c2ccccc2S5)cc1 ; c1ccc(-c2cccc3c2c2c4cccc5c4n(c2n3-c2cccc(-c3cccc4c3oc3ccccc34)c2)-c2ccccc2S5)cc1 ; c1ccc(-c2cccc3c2c2c4cccc5c4n(c2n3-c2cccc(-c3ccc4c(c3)sc3ccccc34)c2)-c2ccccc2S5)cc1]

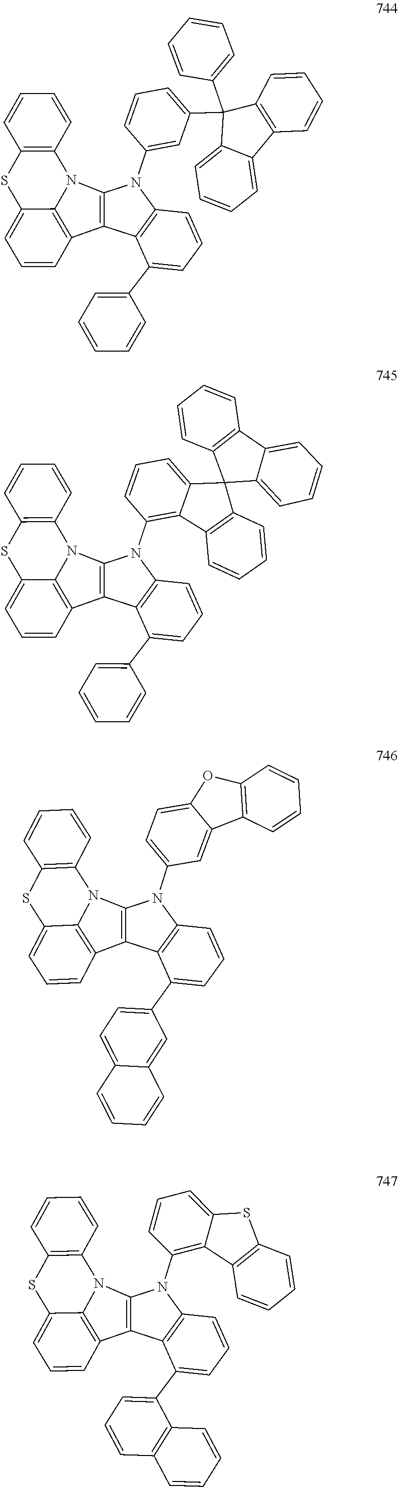 [SMILES: c1ccc(-c2cccc3c2c2c4cccc5c4n(c2n3-c2cccc(C3(c4ccccc4)c4ccccc4-c4ccccc43)c2)-c2ccccc2S5)cc1 ; c1ccc(-c2cccc3c2c2c4cccc5c4n(c2n3-c2cccc3c2-c2ccccc2C32c3ccccc3-c3ccccc32)-c2ccccc2S5)cc1 ; c1ccc2c(c1)Sc1cccc3c4c5c(-c6ccc7ccccc7c6)cccc5n(-c5ccc6oc7ccccc7c6c5)c4n-2c13 ; c1ccc2c(c1)Sc1cccc3c4c5c(-c6cccc7ccccc67)cccc5n(-c5cccc6sc7ccccc7c56)c4n-2c13]

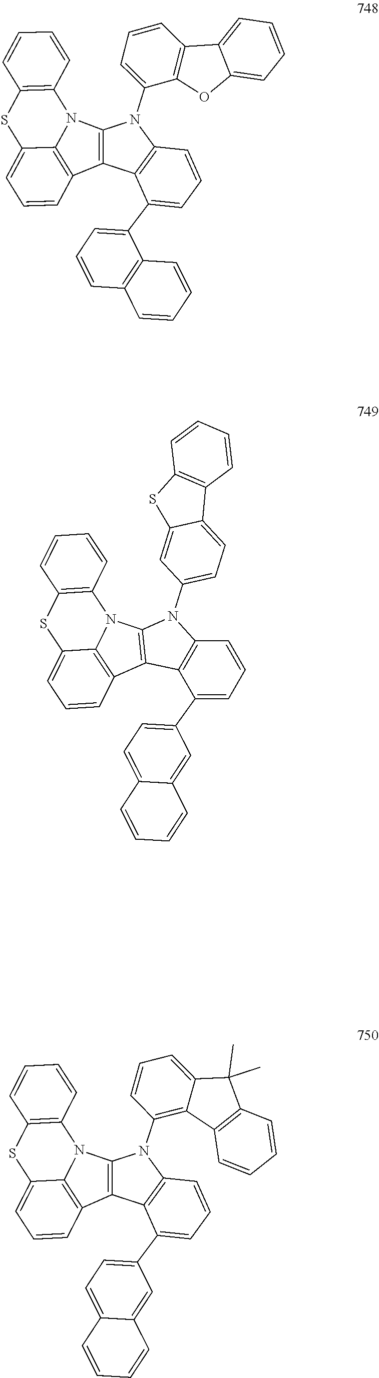 [SMILES: c1ccc2c(c1)Sc1cccc3c4c5c(-c6cccc7ccccc67)cccc5n(-c5cccc6c5oc5ccccc56)c4n-2c13 ; c1ccc2c(c1)Sc1cccc3c4c5c(-c6ccc7ccccc7c6)cccc5n(-c5ccc6c(c5)sc5ccccc56)c4n-2c13 ; CC1(C)c2ccccc2-c2c(-n3c4cccc(-c5ccc6ccccc6c5)c4c4c5cccc6c5n(c43)-c3ccccc3S6)cccc21]

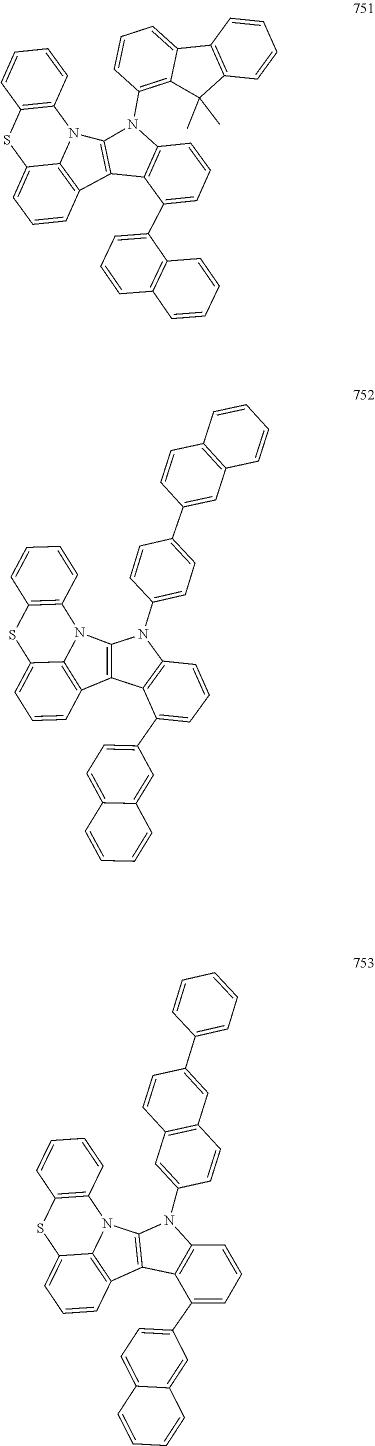 [SMILES: CC1(C)c2ccccc2-c2cccc(-n3c4cccc(-c5cccc6ccccc56)c4c4c5cccc6c5n(c43)-c3ccccc3S6)c21 ; c1ccc2c(c1)Sc1cccc3c4c5c(-c6ccc7ccccc7c6)cccc5n(-c5ccc(-c6ccc7ccccc7c6)cc5)c4n-2c13 ; c1ccc(-c2ccc3cc(-n4c5cccc(-c6ccc7ccccc7c6)c5c5c6cccc7c6n(c54)-c4ccccc4S7)ccc3c2)cc1]

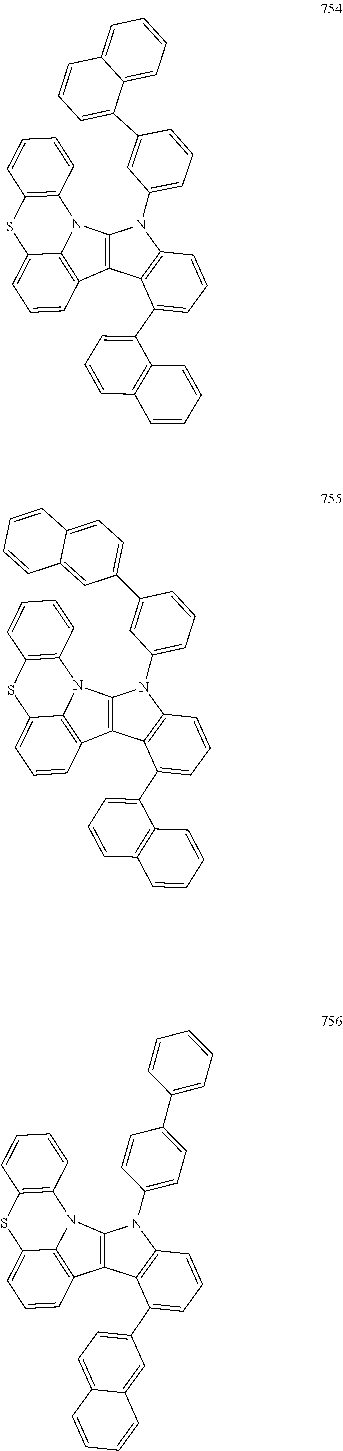 [SMILES: c1cc(-c2cccc3ccccc23)cc(-n2c3cccc(-c4cccc5ccccc45)c3c3c4cccc5c4n(c32)-c2ccccc2S5)c1 ; c1cc(-c2ccc3ccccc3c2)cc(-n2c3cccc(-c4cccc5ccccc45)c3c3c4cccc5c4n(c32)-c2ccccc2S5)c1 ; c1ccc(-c2ccc(-n3c4cccc(-c5ccc6ccccc6c5)c4c4c5cccc6c5n(c43)-c3ccccc3S6)cc2)cc1]

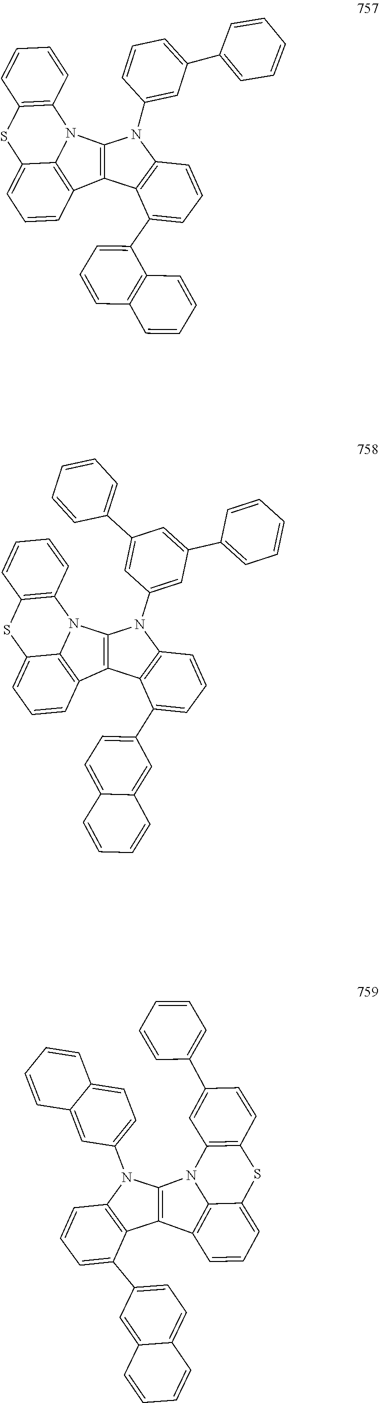 [SMILES: c1ccc(-c2cccc(-n3c4cccc(-c5cccc6ccccc56)c4c4c5cccc6c5n(c43)-c3ccccc3S6)c2)cc1 ; c1ccc(-c2cc(-c3ccccc3)cc(-n3c4cccc(-c5ccc6ccccc6c5)c4c4c5cccc6c5n(c43)-c3ccccc3S6)c2)cc1 ; c1ccc(-c2ccc3c(c2)-n2c4c(cccc4c4c5c(-c6ccc7ccccc7c6)cccc5n(-c5ccc6ccccc6c5)c42)S3)cc1]

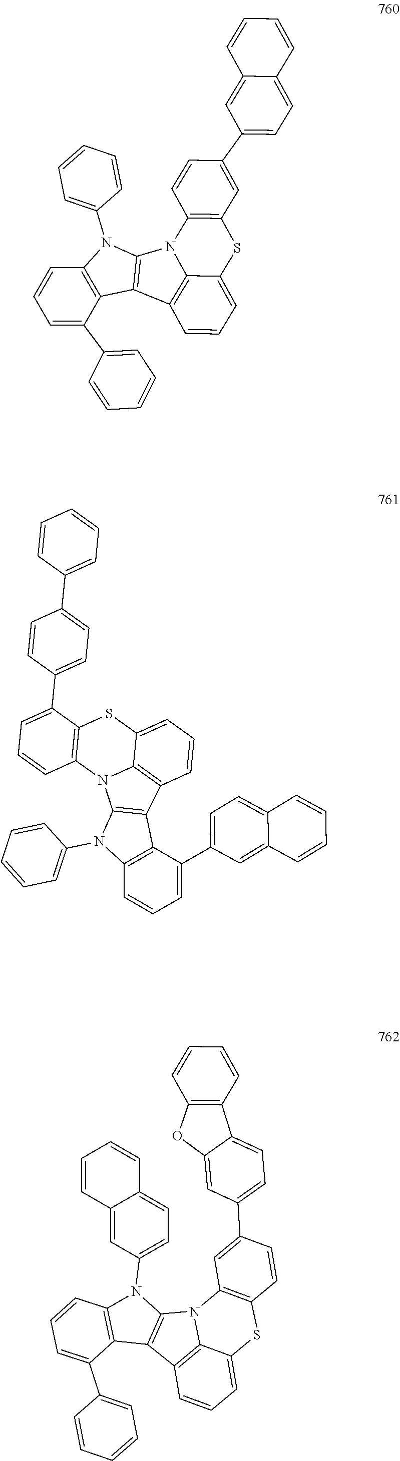 [SMILES: c1ccc(-c2cccc3c2c2c4cccc5c4n(c2n3-c2ccccc2)-c2ccc(-c3ccc4ccccc4c3)cc2S5)cc1 ; c1ccc(-c2ccc(-c3cccc4c3Sc3cccc5c6c7c(-c8ccc9ccccc9c8)cccc7n(-c7ccccc7)c6n-4c35)cc2)cc1 ; c1ccc(-c2cccc3c2c2c4cccc5c4n(c2n3-c2ccc3ccccc3c2)-c2cc(-c3ccc4c(c3)oc3ccccc34)ccc2S5)cc1]

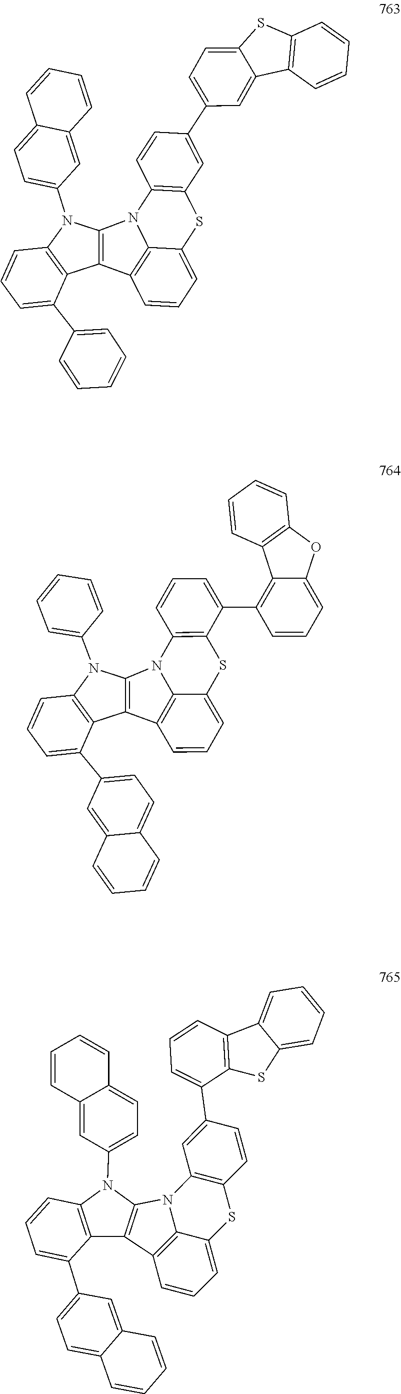 [SMILES: c1ccc(-c2cccc3c2c2c4cccc5c4n(c2n3-c2ccc3ccccc3c2)-c2ccc(-c3ccc4sc6ccccc6c4c3)cc2S5)cc1 ; c1ccc(-n2c3cccc(-c4ccc5ccccc5c4)c3c3c4cccc5c4n(c32)-c2cccc(-c3cccc4oc6ccccc6c34)c2S5)cc1 ; c1ccc2cc(-c3cccc4c3c3c5cccc6c5n(c3n4-c3ccc4ccccc4c3)-c3cc(-c4cccc5c4sc4ccccc45)ccc3S6)ccc2c1]

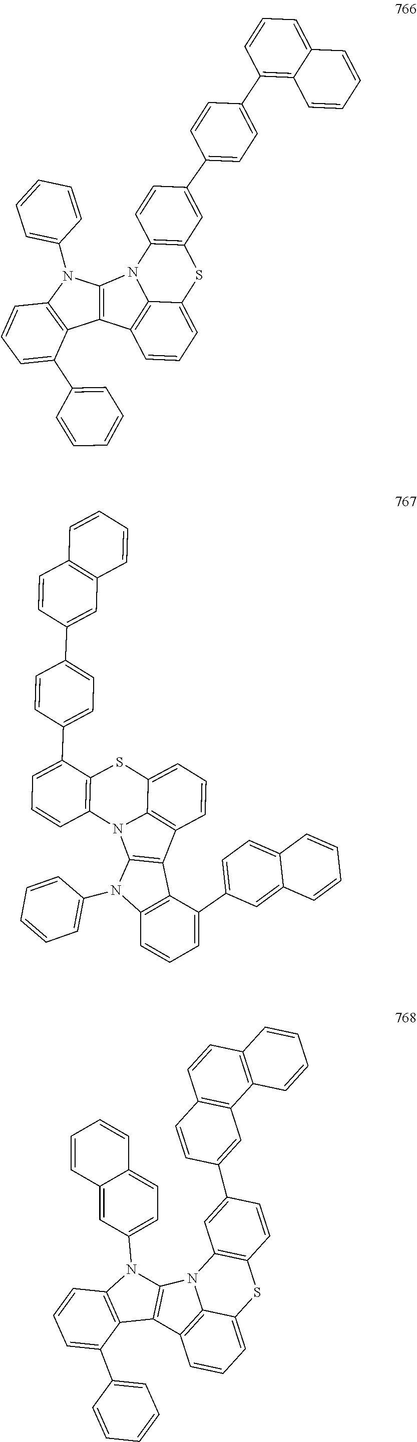 [SMILES: c1ccc(-c2cccc3c2c2c4cccc5c4n(c2n3-c2ccccc2)-c2ccc(-c3ccc(-c4cccc6ccccc46)cc3)cc2S5)cc1 ; c1ccc(-n2c3cccc(-c4ccc5ccccc5c4)c3c3c4cccc5c4n(c32)-c2cccc(-c3ccc(-c4ccc6ccccc6c4)cc3)c2S5)cc1 ; c1ccc(-c2cccc3c2c2c4cccc5c4n(c2n3-c2ccc3ccccc3c2)-c2cc(-c3ccc4ccc6ccccc6c4c3)ccc2S5)cc1]

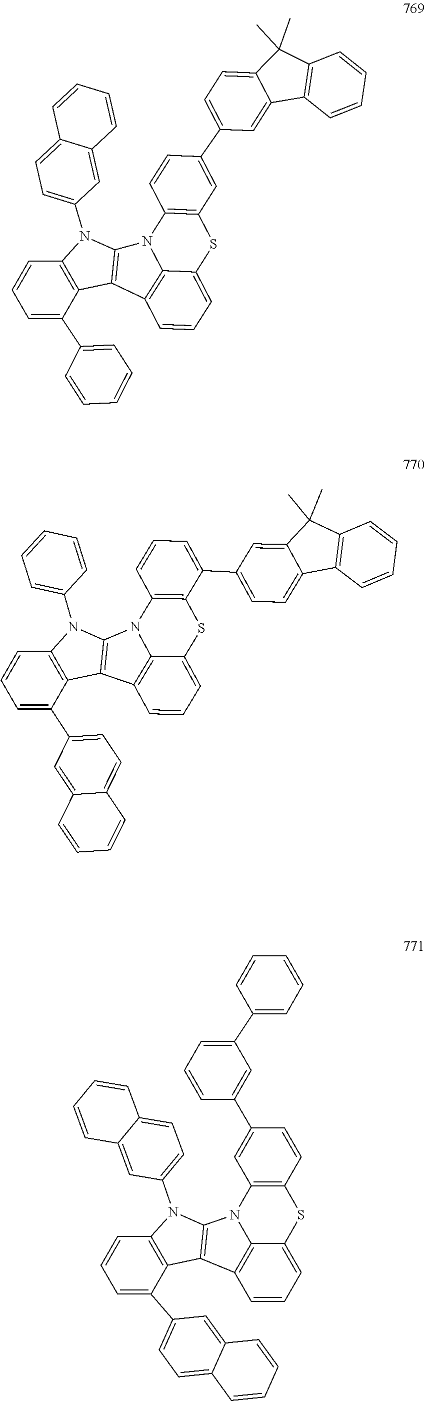 [SMILES: CC1(C)c2ccccc2-c2cc(-c3ccc4c(c3)Sc3cccc5c6c7c(-c8ccccc8)cccc7n(-c7ccc8ccccc8c7)c6n-4c35)ccc21 ; CC1(C)c2ccccc2-c2ccc(-c3cccc4c3Sc3cccc5c6c7c(-c8ccc9ccccc9c8)cccc7n(-c7ccccc7)c6n-4c35)cc21 ; c1ccc(-c2cccc(-c3ccc4c(c3)-n3c5c(cccc5c5c6c(-c7ccc8ccccc8c7)cccc6n(-c6ccc7ccccc7c6)c53)S4)c2)cc1]

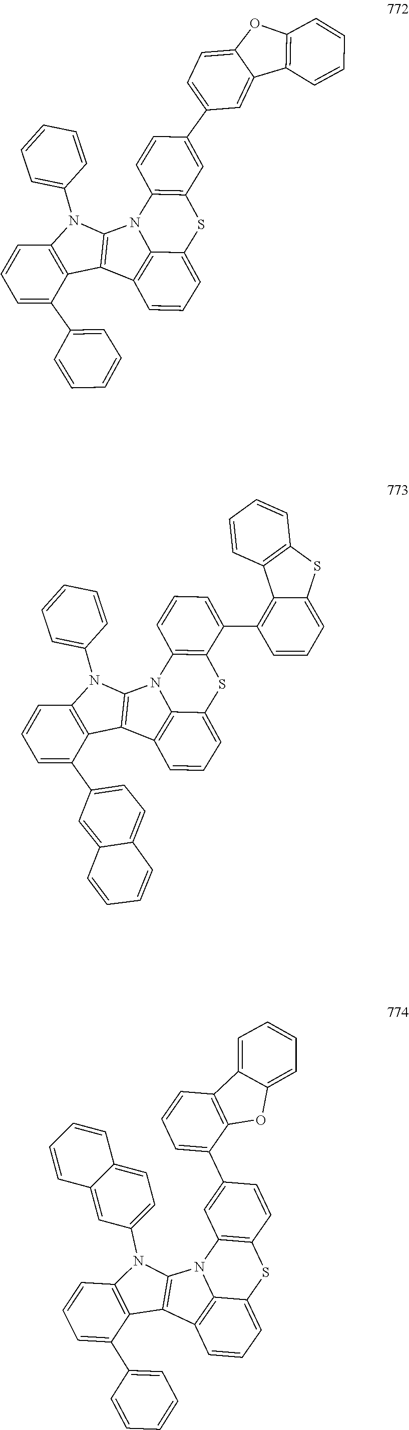 [SMILES: c1ccc(-c2cccc3c2c2c4cccc5c4n(c2n3-c2ccccc2)-c2ccc(-c3ccc4oc6ccccc6c4c3)cc2S5)cc1 ; c1ccc(-n2c3cccc(-c4ccc5ccccc5c4)c3c3c4cccc5c4n(c32)-c2cccc(-c3cccc4sc6ccccc6c34)c2S5)cc1 ; c1ccc(-c2cccc3c2c2c4cccc5c4n(c2n3-c2ccc3ccccc3c2)-c2cc(-c3cccc4c3oc3ccccc34)ccc2S5)cc1]

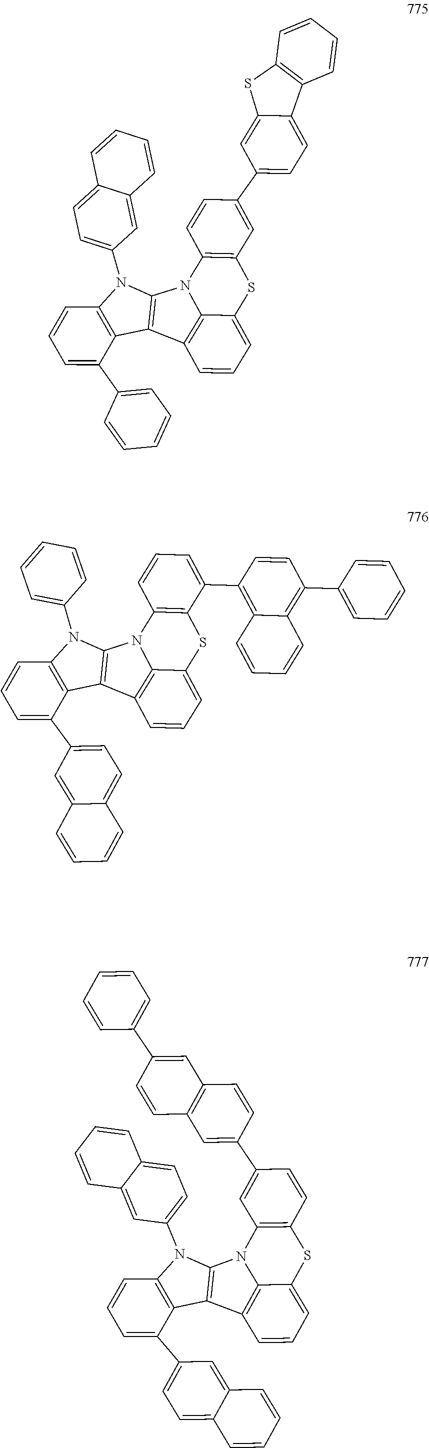 [SMILES: c1ccc(-c2cccc3c2c2c4cccc5c4n(c2n3-c2ccc3ccccc3c2)-c2ccc(-c3ccc4c(c3)sc3ccccc34)cc2S5)cc1 ; c1ccc(-c2ccc(-c3cccc4c3Sc3cccc5c6c7c(-c8ccc9ccccc9c8)cccc7n(-c7ccccc7)c6n-4c35)c3ccccc23)cc1 ; c1ccc(-c2ccc3cc(-c4ccc5c(c4)-n4c6c(cccc6c6c7c(-c8ccc9ccccc9c8)cccc7n(-c7ccc8ccccc8c7)c64)S5)ccc3c2)cc1]

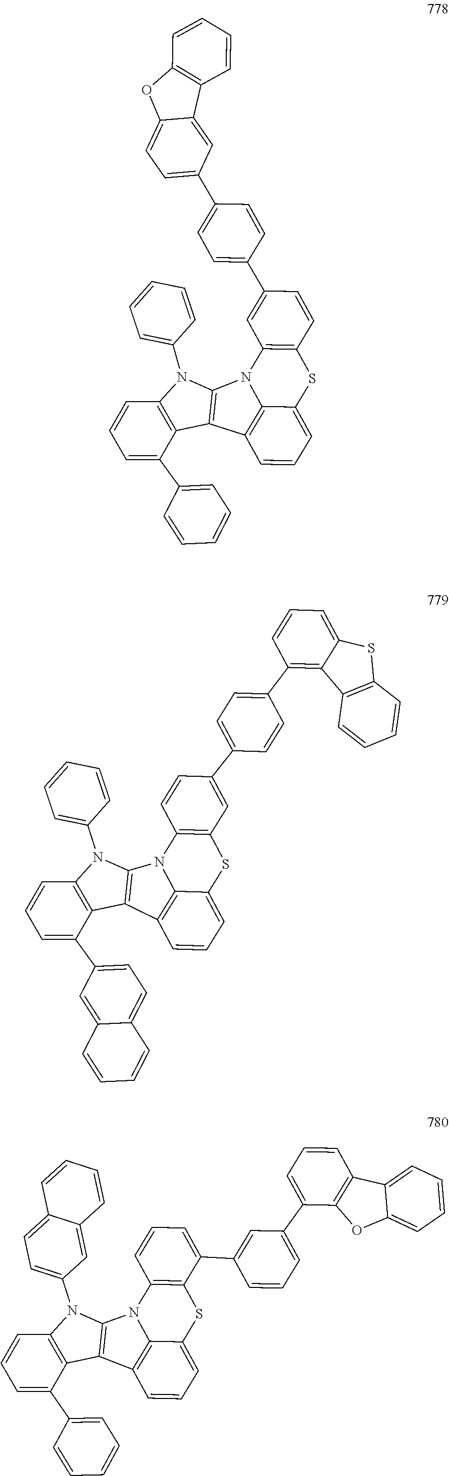 [SMILES: c1ccc(-c2cccc3c2c2c4cccc5c4n(c2n3-c2ccccc2)-c2cc(-c3ccc(-c4ccc6oc7ccccc7c6c4)cc3)ccc2S5)cc1 ; c1ccc(-n2c3cccc(-c4ccc5ccccc5c4)c3c3c4cccc5c4n(c32)-c2ccc(-c3ccc(-c4cccc6sc7ccccc7c46)cc3)cc2S5)cc1 ; c1ccc(-c2cccc3c2c2c4cccc5c4n(c2n3-c2ccc3ccccc3c2)-c2cccc(-c3cccc(-c4cccc6c4oc4ccccc46)c3)c2S5)cc1]

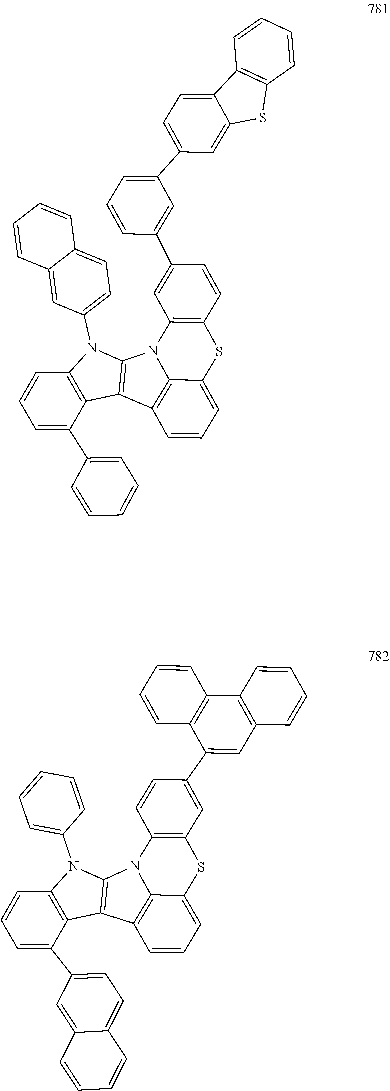 [SMILES: c1ccc(-c2cccc3c2c2c4cccc5c4n(c2n3-c2ccc3ccccc3c2)-c2cc(-c3cccc(-c4ccc6c(c4)sc4ccccc46)c3)ccc2S5)cc1 ; c1ccc(-n2c3cccc(-c4ccc5ccccc5c4)c3c3c4cccc5c4n(c32)-c2ccc(-c3cc4ccccc4c4ccccc34)cc2S5)cc1]

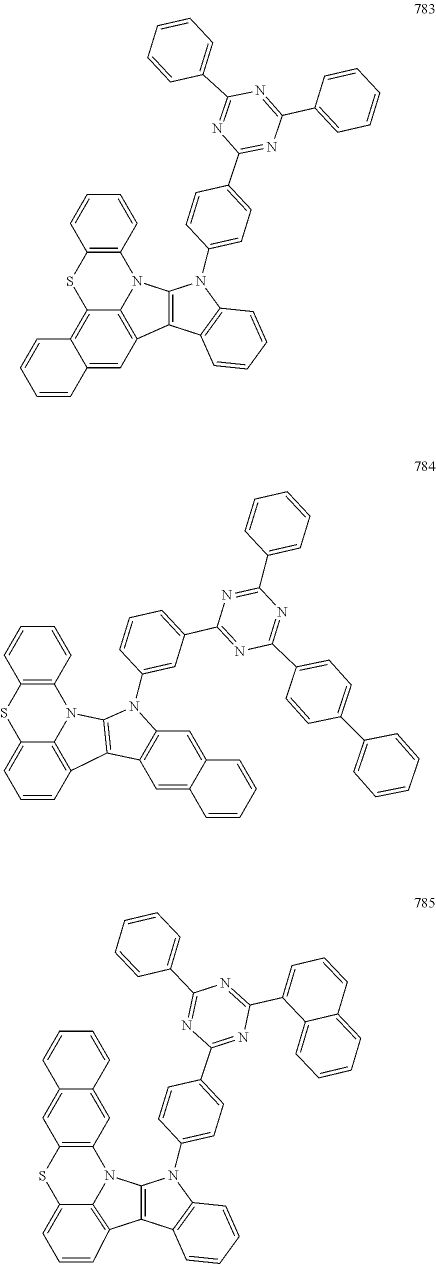 [SMILES: c1ccc(-c2nc(-c3ccccc3)nc(-c3ccc(-n4c5ccccc5c5c6cc7ccccc7c7c6n(c54)-c4ccccc4S7)cc3)n2)cc1 ; c1ccc(-c2ccc(-c3nc(-c4ccccc4)nc(-c4cccc(-n5c6cc7ccccc7cc6c6c7cccc8c7n(c65)-c5ccccc5S8)c4)n3)cc2)cc1 ; c1ccc(-c2nc(-c3ccc(-n4c5ccccc5c5c6cccc7c6n(c54)-c4cc5ccccc5cc4S7)cc3)nc(-c3cccc4ccccc34)n2)cc1]

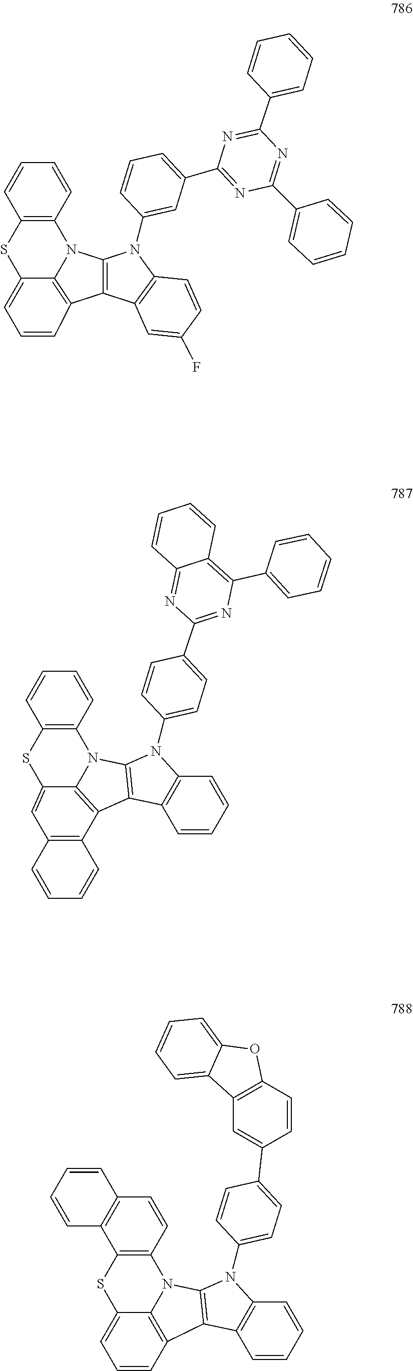 [SMILES: Fc1ccc2c(c1)c1c3cccc4c3n(c1n2-c1cccc(-c2nc(-c3ccccc3)nc(-c3ccccc3)n2)c1)-c1ccccc1S4 ; c1ccc(-c2nc(-c3ccc(-n4c5ccccc5c5c6c7ccccc7cc7c6n(c54)-c4ccccc4S7)cc3)nc3ccccc23)cc1 ; c1ccc2c3c(ccc2c1)-n1c2c(cccc2c2c4ccccc4n(-c4ccc(-c5ccc6oc7ccccc7c6c5)cc4)c21)S3]

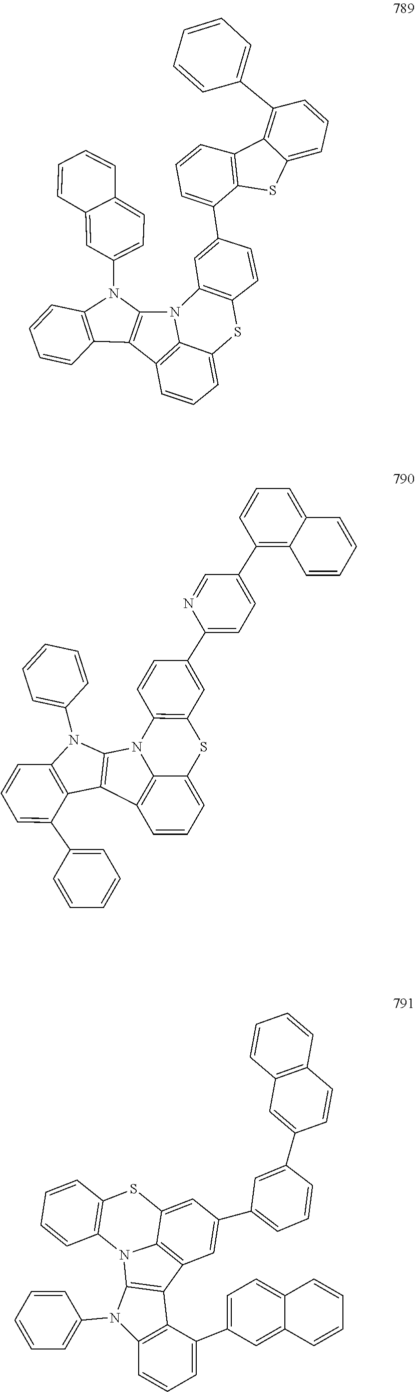

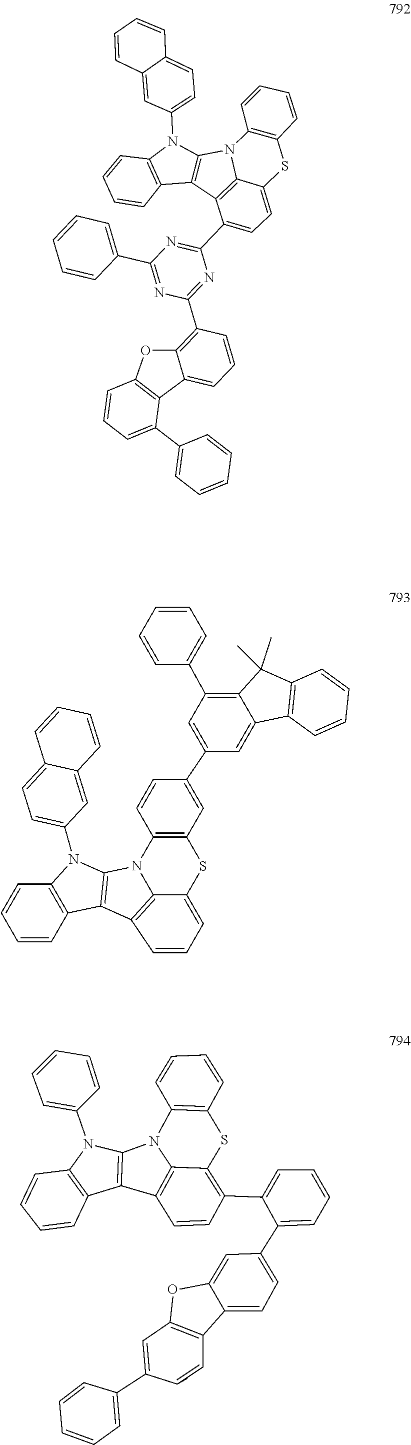

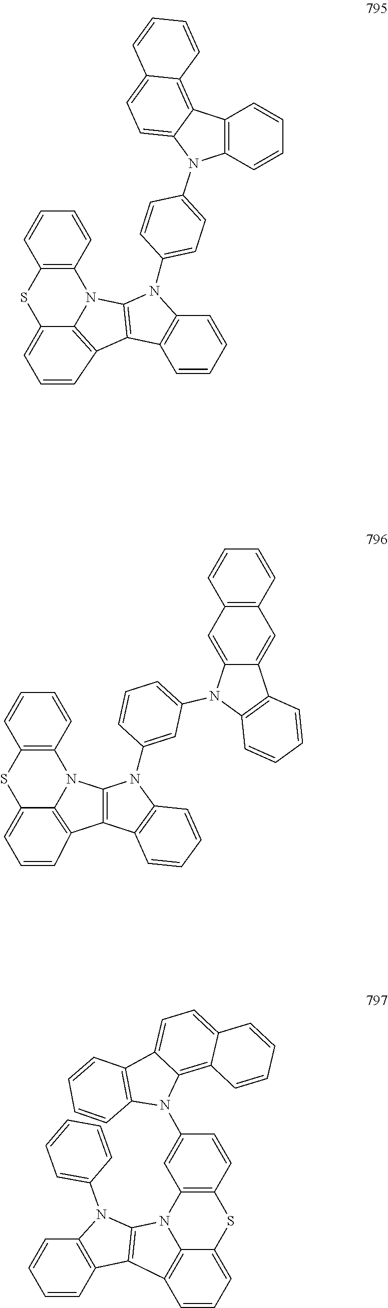 [SMILES: c1ccc2c(c1)Sc1cccc3c4c5ccccc5n(-c5ccc(-n6c7ccccc7c7c8ccccc8ccc76)cc5)c4n-2c13 ; c1cc(-n2c3ccccc3c3cc4ccccc4cc32)cc(-n2c3ccccc3c3c4cccc5c4n(c32)-c2ccccc2S5)c1 ; c1ccc(-n2c3ccccc3c3c4cccc5c4n(c32)-c2cc(-n3c4ccccc4c4ccc6ccccc6c43)ccc2S5)cc1]

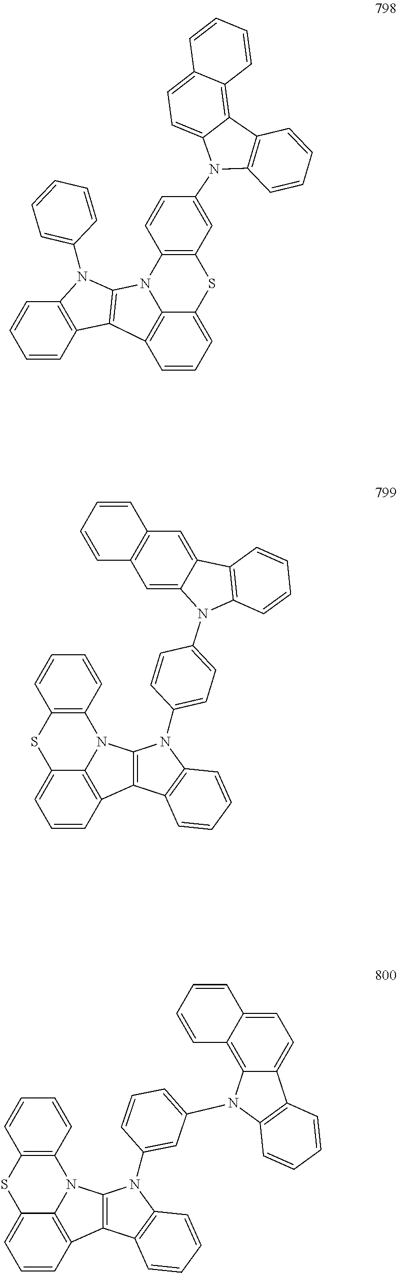 [SMILES: c1ccc(-n2c3ccccc3c3c4cccc5c4n(c32)-c2ccc(-n3c4ccccc4c4c6ccccc6ccc43)cc2S5)cc1 ; c1ccc2c(c1)Sc1cccc3c4c5ccccc5n(-c5ccc(-n6c7ccccc7c7cc8ccccc8cc76)cc5)c4n-2c13 ; c1cc(-n2c3ccccc3c3ccc4ccccc4c32)cc(-n2c3ccccc3c3c4cccc5c4n(c32)-c2ccccc2S5)c1]

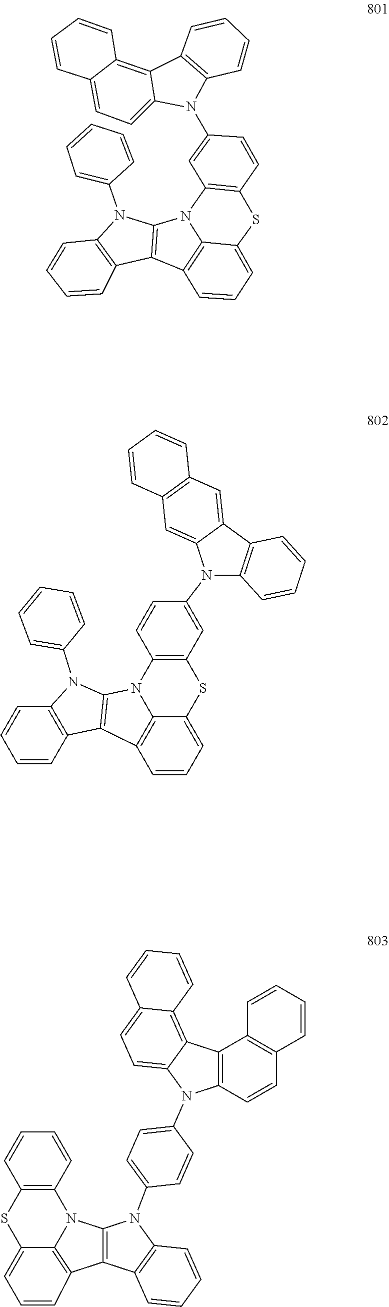 [SMILES: c1ccc(-n2c3ccccc3c3c4cccc5c4n(c32)-c2cc(-n3c4ccccc4c4c6ccccc6ccc43)ccc2S5)cc1 ; c1ccc(-n2c3ccccc3c3c4cccc5c4n(c32)-c2ccc(-n3c4ccccc4c4cc6ccccc6cc43)cc2S5)cc1 ; c1ccc2c(c1)Sc1cccc3c4c5ccccc5n(-c5ccc(-n6c7ccc8ccccc8c7c7c8ccccc8ccc76)cc5)c4n-2c13]

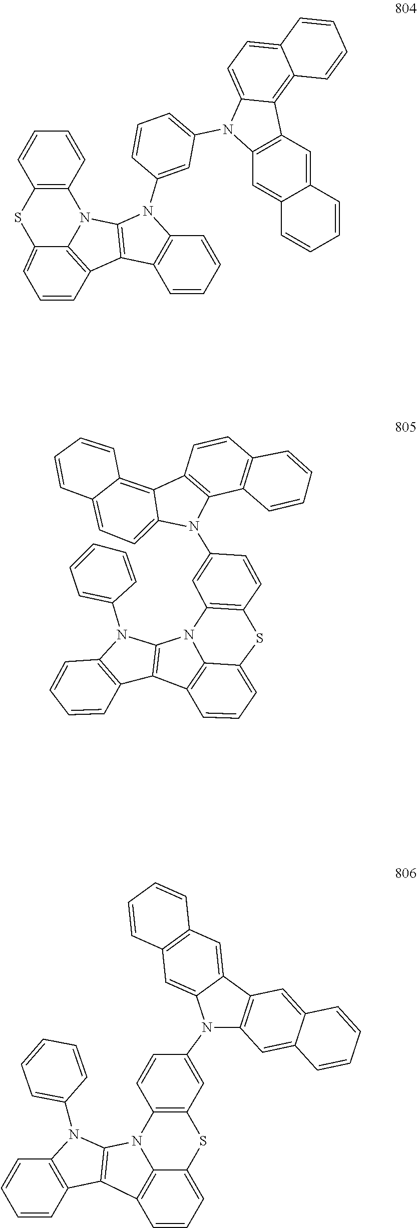 [SMILES: c1cc(-n2c3cc4ccccc4cc3c3c4ccccc4ccc32)cc(-n2c3ccccc3c3c4cccc5c4n(c32)-c2ccccc2S5)c1 ; c1ccc(-n2c3ccccc3c3c4cccc5c4n(c32)-c2cc(-n3c4ccc6ccccc6c4c4ccc6ccccc6c43)ccc2S5)cc1 ; c1ccc(-n2c3ccccc3c3c4cccc5c4n(c32)-c2ccc(-n3c4cc6ccccc6cc4c4cc6ccccc6cc43)cc2S5)cc1]

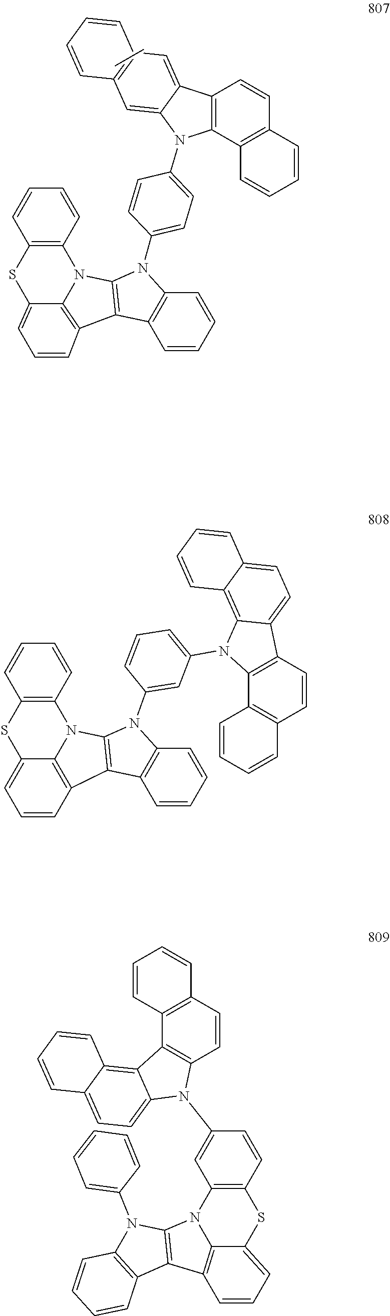

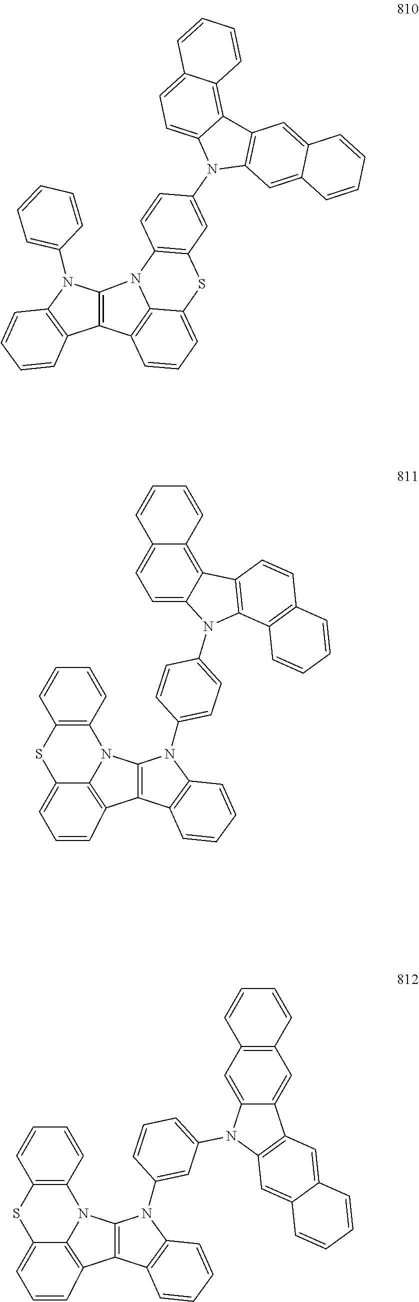 [SMILES: c1ccc(-n2c3ccccc3c3c4cccc5c4n(c32)-c2ccc(-n3c4cc6ccccc6cc4c4c6ccccc6ccc43)cc2S5)cc1 ; c1ccc2c(c1)Sc1cccc3c4c5ccccc5n(-c5ccc(-n6c7ccc8ccccc8c7c7ccc8ccccc8c76)cc5)c4n-2c13 ; c1cc(-n2c3cc4ccccc4cc3c3cc4ccccc4cc32)cc(-n2c3ccccc3c3c4cccc5c4n(c32)-c2ccccc2S5)c1]

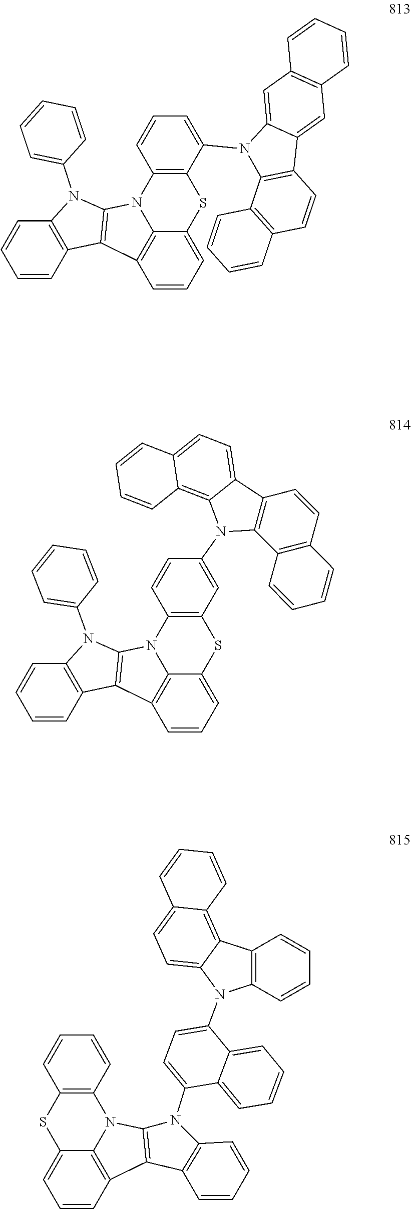 [SMILES: c1ccc(-n2c3ccccc3c3c4cccc5c4n(c32)-c2cccc(-n3c4cc6ccccc6cc4c4ccc6ccccc6c43)c2S5)cc1 ; c1ccc(-n2c3ccccc3c3c4cccc5c4n(c32)-c2ccc(-n3c4c6ccccc6ccc4c4ccc6ccccc6c43)cc2S5)cc1 ; c1ccc2c(c1)Sc1cccc3c4c5ccccc5n(-c5ccc(-n6c7ccccc7c7c8ccccc8ccc76)c6ccccc56)c4n-2c13]

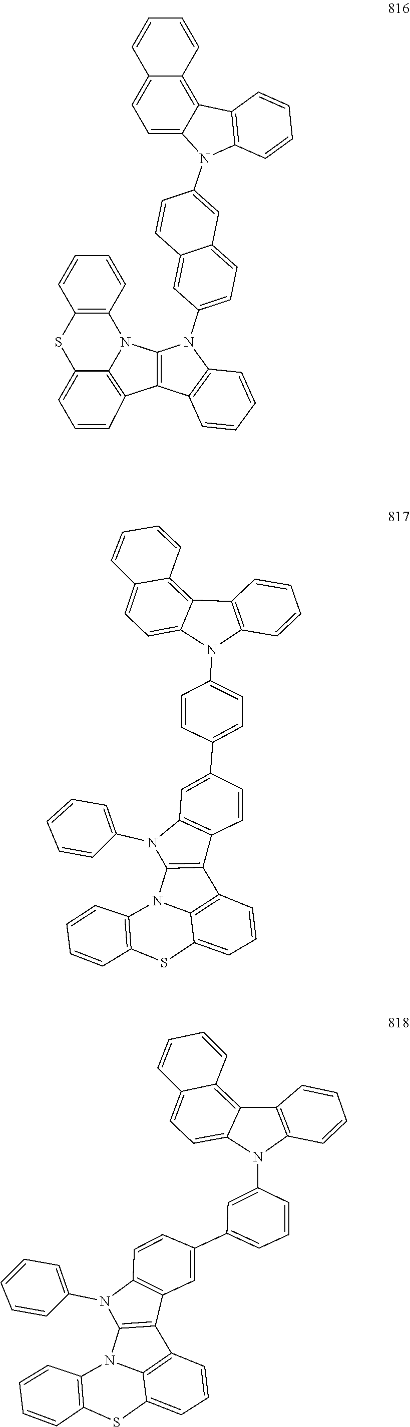 [SMILES: c1ccc2c(c1)Sc1cccc3c4c5ccccc5n(-c5ccc6cc(-n7c8ccccc8c8c9ccccc9ccc87)ccc6c5)c4n-2c13 ; c1ccc(-n2c3cc(-c4ccc(-n5c6ccccc6c6c7ccccc7ccc65)cc4)ccc3c3c4cccc5c4n(c32)-c2ccccc2S5)cc1 ; c1ccc(-n2c3ccc(-c4cccc(-n5c6ccccc6c6c7ccccc7ccc65)c4)cc3c3c4cccc5c4n(c32)-c2ccccc2S5)cc1]

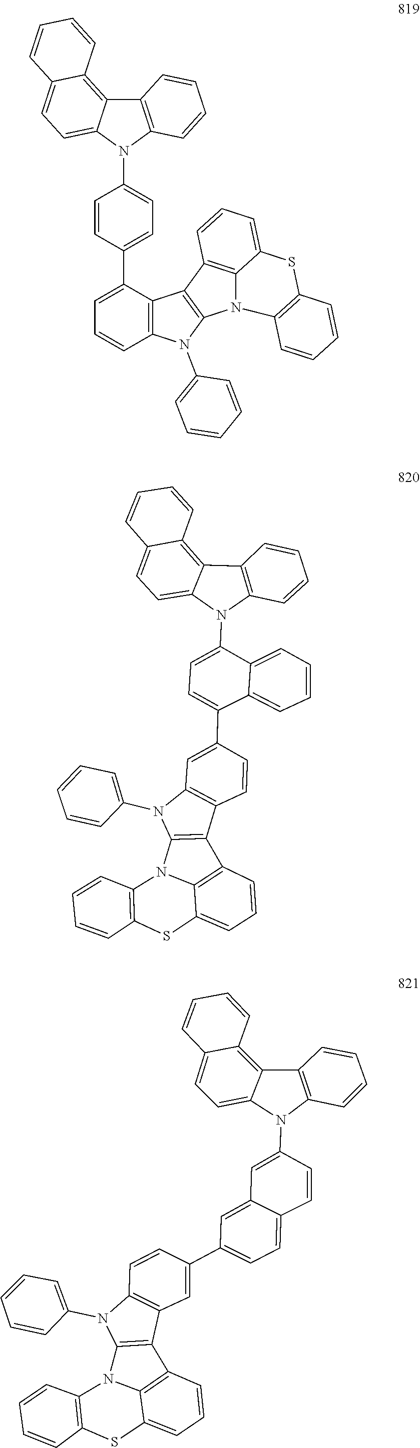 [SMILES: c1ccc(-n2c3cccc(-c4ccc(-n5c6ccccc6c6c7ccccc7ccc65)cc4)c3c3c4cccc5c4n(c32)-c2ccccc2S5)cc1 ; c1ccc(-n2c3cc(-c4ccc(-n5c6ccccc6c6c7ccccc7ccc65)c5ccccc45)ccc3c3c4cccc5c4n(c32)-c2ccccc2S5)cc1 ; c1ccc(-n2c3ccc(-c4ccc5ccc(-n6c7ccccc7c7c8ccccc8ccc76)cc5c4)cc3c3c4cccc5c4n(c32)-c2ccccc2S5)cc1]

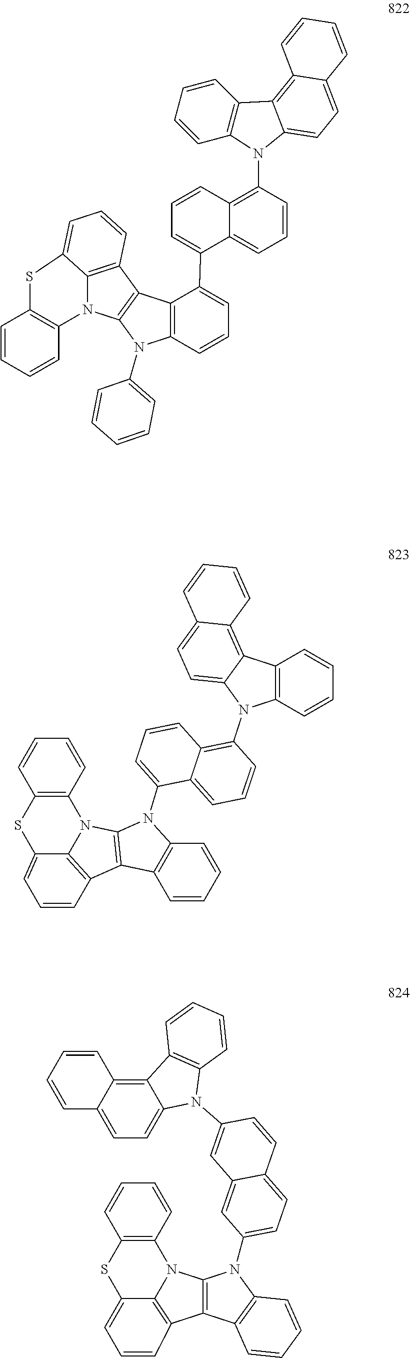 [SMILES: c1ccc(-n2c3cccc(-c4cccc5c(-n6c7ccccc7c7c8ccccc8ccc76)cccc45)c3c3c4cccc5c4n(c32)-c2ccccc2S5)cc1 ; c1ccc2c(c1)Sc1cccc3c4c5ccccc5n(-c5cccc6c(-n7c8ccccc8c8c9ccccc9ccc87)cccc56)c4n-2c13 ; c1ccc2c(c1)Sc1cccc3c4c5ccccc5n(-c5ccc6ccc(-n7c8ccccc8c8c9ccccc9ccc87)cc6c5)c4n-2c13]

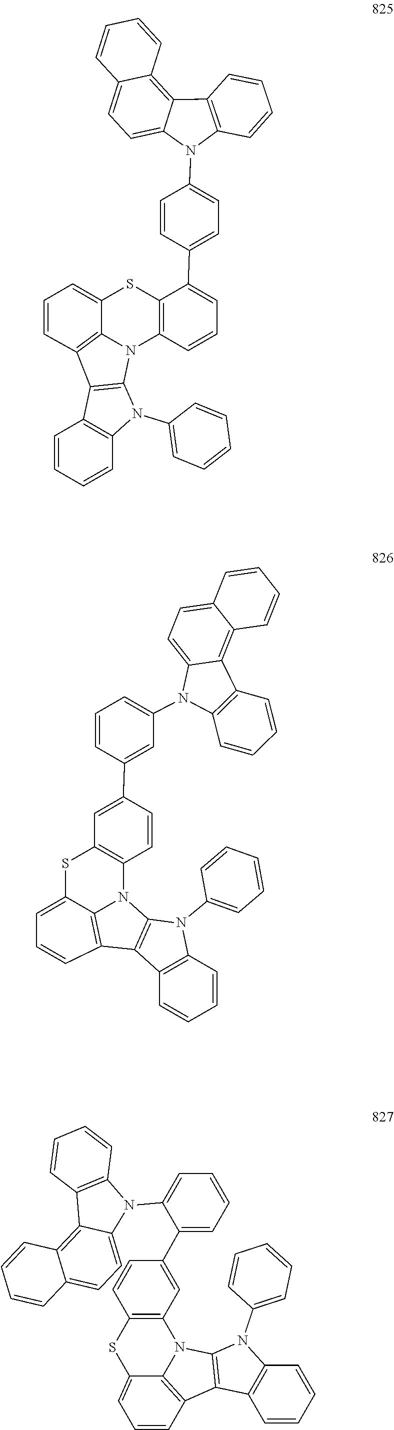 [SMILES: c1ccc(-n2c3ccccc3c3c4cccc5c4n(c32)-c2cccc(-c3ccc(-n4c6ccccc6c6c7ccccc7ccc64)cc3)c2S5)cc1 ; c1ccc(-n2c3ccccc3c3c4cccc5c4n(c32)-c2ccc(-c3cccc(-n4c6ccccc6c6c7ccccc7ccc64)c3)cc2S5)cc1 ; c1ccc(-n2c3ccccc3c3c4cccc5c4n(c32)-c2cc(-c3ccccc3-n3c4ccccc4c4c6ccccc6ccc43)ccc2S5)cc1]

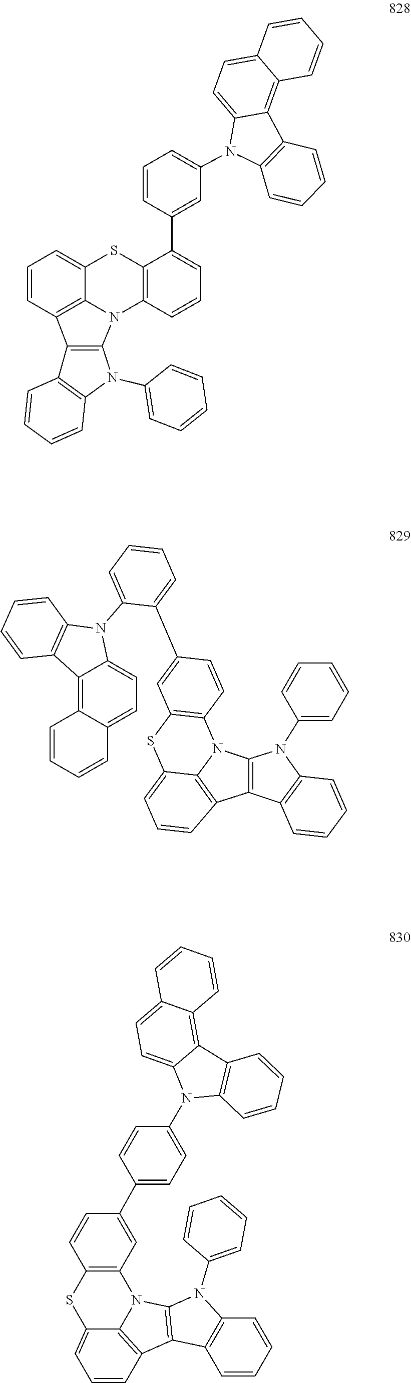 [SMILES: c1ccc(-n2c3ccccc3c3c4cccc5c4n(c32)-c2cccc(-c3cccc(-n4c6ccccc6c6c7ccccc7ccc64)c3)c2S5)cc1 ; c1ccc(-n2c3ccccc3c3c4cccc5c4n(c32)-c2ccc(-c3ccccc3-n3c4ccccc4c4c6ccccc6ccc43)cc2S5)cc1 ; c1ccc(-n2c3ccccc3c3c4cccc5c4n(c32)-c2cc(-c3ccc(-n4c6ccccc6c6c7ccccc7ccc64)cc3)ccc2S5)cc1]

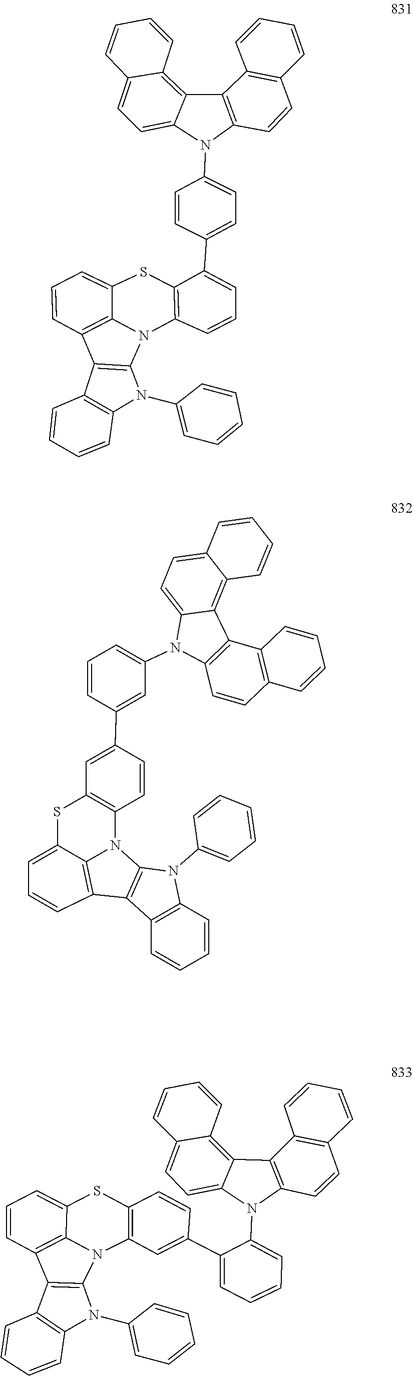 [SMILES: c1ccc(-n2c3ccccc3c3c4cccc5c4n(c32)-c2cccc(-c3ccc(-n4c6ccc7ccccc7c6c6c7ccccc7ccc64)cc3)c2S5)cc1 ; c1ccc(-n2c3ccccc3c3c4cccc5c4n(c32)-c2ccc(-c3cccc(-n4c6ccc7ccccc7c6c6c7ccccc7ccc64)c3)cc2S5)cc1 ; c1ccc(-n2c3ccccc3c3c4cccc5c4n(c32)-c2cc(-c3ccccc3-n3c4ccc6ccccc6c4c4c6ccccc6ccc43)ccc2S5)cc1]

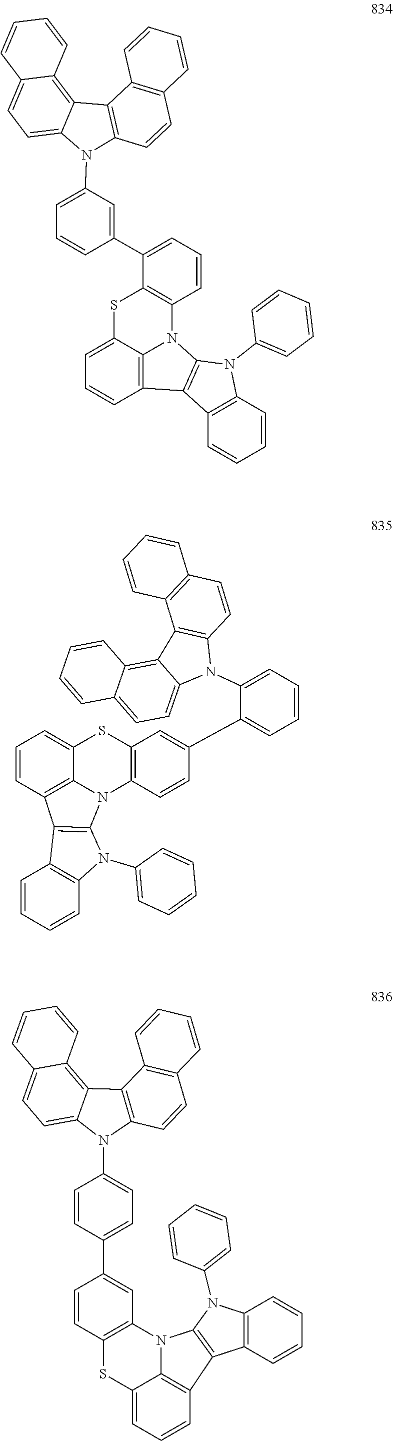 [SMILES: c1ccc(-n2c3ccccc3c3c4cccc5c4n(c32)-c2cccc(-c3cccc(-n4c6ccc7ccccc7c6c6c7ccccc7ccc64)c3)c2S5)cc1 ; c1ccc(-n2c3ccccc3c3c4cccc5c4n(c32)-c2ccc(-c3ccccc3-n3c4ccc6ccccc6c4c4c6ccccc6ccc43)cc2S5)cc1 ; c1ccc(-n2c3ccccc3c3c4cccc5c4n(c32)-c2cc(-c3ccc(-n4c6ccc7ccccc7c6c6c7ccccc7ccc64)cc3)ccc2S5)cc1]

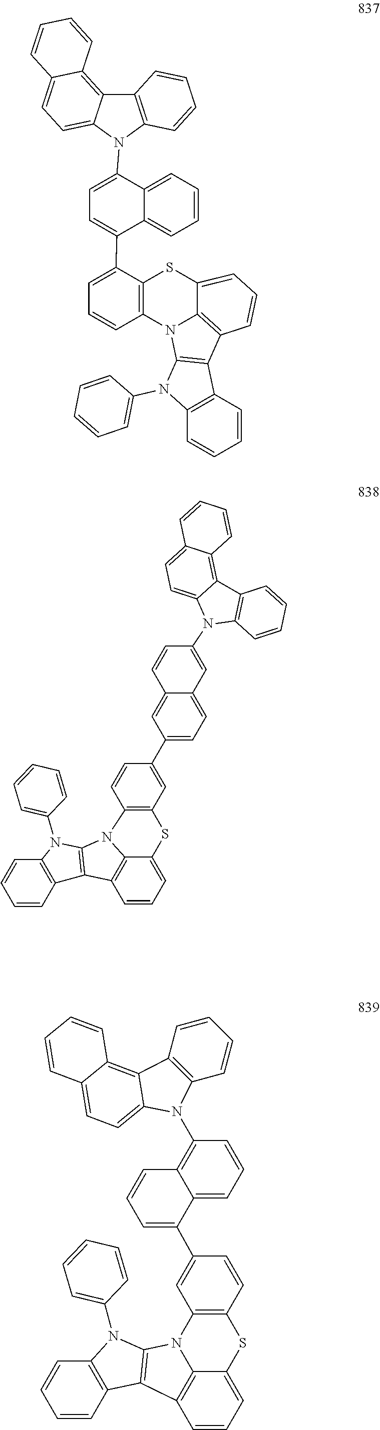 [SMILES: c1ccc(-n2c3ccccc3c3c4cccc5c4n(c32)-c2cccc(-c3ccc(-n4c6ccccc6c6c7ccccc7ccc64)c4ccccc34)c2S5)cc1 ; c1ccc(-n2c3ccccc3c3c4cccc5c4n(c32)-c2ccc(-c3ccc4cc(-n6c7ccccc7c7c8ccccc8ccc76)ccc4c3)cc2S5)cc1 ; c1ccc(-n2c3ccccc3c3c4cccc5c4n(c32)-c2cc(-c3cccc4c(-n6c7ccccc7c7c8ccccc8ccc76)cccc34)ccc2S5)cc1]

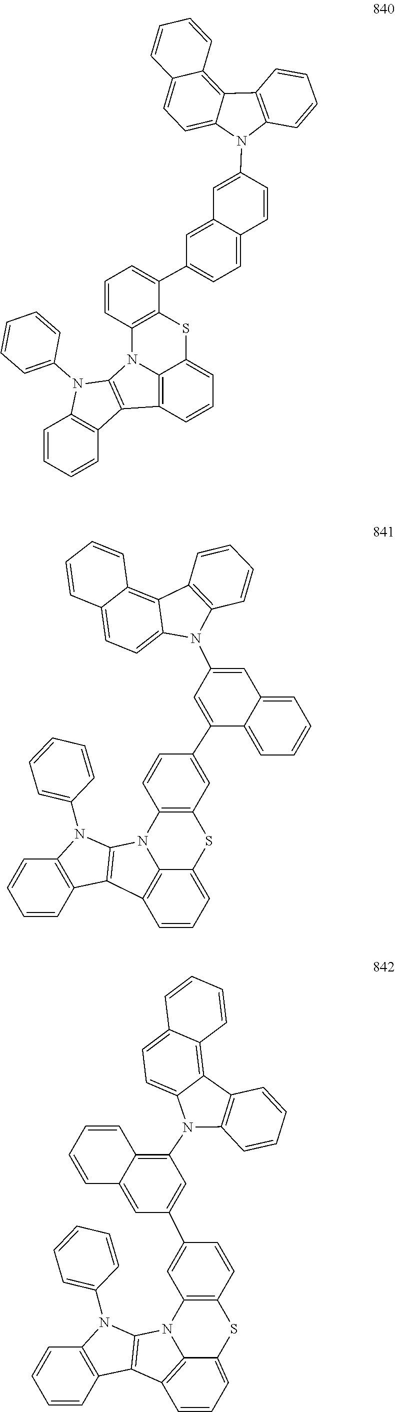 [SMILES: c1ccc(-n2c3ccccc3c3c4cccc5c4n(c32)-c2cccc(-c3ccc4ccc(-n6c7ccccc7c7c8ccccc8ccc76)cc4c3)c2S5)cc1 ; c1ccc(-n2c3ccccc3c3c4cccc5c4n(c32)-c2ccc(-c3cc(-n4c6ccccc6c6c7ccccc7ccc64)cc4ccccc34)cc2S5)cc1 ; c1ccc(-n2c3ccccc3c3c4cccc5c4n(c32)-c2cc(-c3cc(-n4c6ccccc6c6c7ccccc7ccc64)c4ccccc4c3)ccc2S5)cc1]

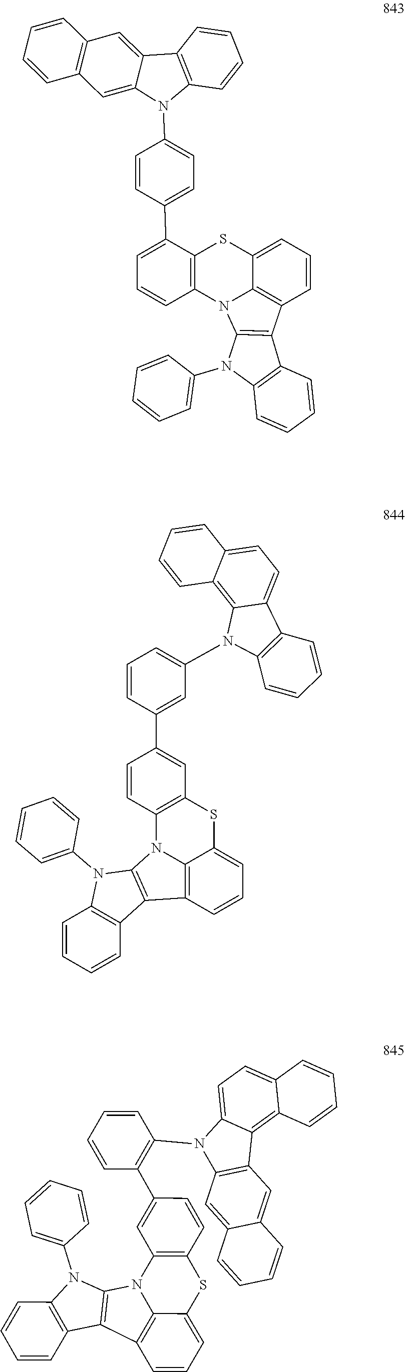 [SMILES: c1ccc(-n2c3ccccc3c3c4cccc5c4n(c32)-c2cccc(-c3ccc(-n4c6ccccc6c6cc7ccccc7cc64)cc3)c2S5)cc1 ; c1ccc(-n2c3ccccc3c3c4cccc5c4n(c32)-c2ccc(-c3cccc(-n4c6ccccc6c6ccc7ccccc7c64)c3)cc2S5)cc1 ; c1ccc(-n2c3ccccc3c3c4cccc5c4n(c32)-c2cc(-c3ccccc3-n3c4cc6ccccc6cc4c4c6ccccc6ccc43)ccc2S5)cc1]

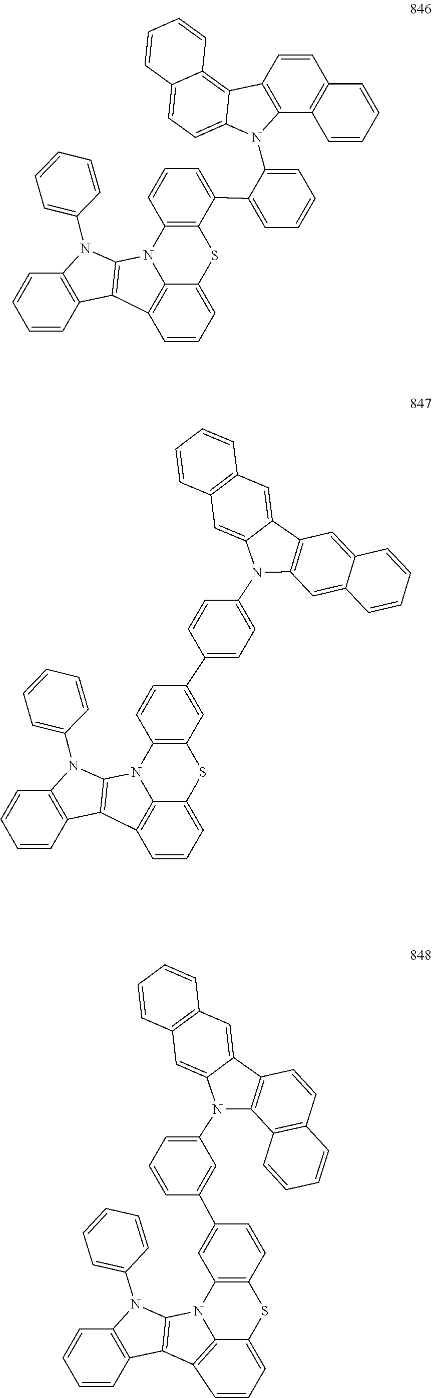 [SMILES: c1ccc(-n2c3ccccc3c3c4cccc5c4n(c32)-c2cccc(-c3ccccc3-n3c4ccc6ccccc6c4c4ccc6ccccc6c43)c2S5)cc1 ; c1ccc(-n2c3ccccc3c3c4cccc5c4n(c32)-c2ccc(-c3ccc(-n4c6cc7ccccc7cc6c6cc7ccccc7cc64)cc3)cc2S5)cc1 ; c1ccc(-n2c3ccccc3c3c4cccc5c4n(c32)-c2cc(-c3cccc(-n4c6cc7ccccc7cc6c6ccc7ccccc7c64)c3)ccc2S5)cc1]

15. An organic electroluminescent device, comprising an anode and a cathode that are disposed opposite to each other, and a functional layer disposed between the anode and the cathode, wherein the functional layer comprises the nitrogen-containing compound according to claim 1; and
optionally, the functional layer comprises an organic luminescent layer, wherein the organic luminescent layer comprises the nitrogen-containing compound.

16. An electronic apparatus, comprising the organic electroluminescent device according to claim 15.
